(19) United States
(12) Patent Application Publication
(10) Pub. No.: US 2023/0063462 A1
(43) Pub. Date: Mar. 2, 2023

(54) SULFONIMIDAMIDE COMPOUNDS AND COMPOSITIONS FOR TREATING CONDITIONS ASSOCIATED WITH NLRP ACTIVITY

(71) Applicant: NOVARTIS AG, Basel (CH)

(72) Inventors: Jason KATZ, Newton, MA (US); William R. ROUSH, Boston, MA (US); Hans Martin SEIDEL, Concord, MA (US); Dong-Ming SHEN, Edison, NJ (US); Shankar VENKATRAMAN, Lansdale, PA (US)

(21) Appl. No.: 17/422,650

(22) PCT Filed: Jan. 23, 2020
(86) PCT No.: PCT/US2020/014788

§ 371 Date: Jul. 13, 2021

Related U.S. Application Data
(60) Provisional application No. 62/795,964, filed on Jan. 23, 2019.
(60) Provisional application No. 62/795,969, filed on Jan. 23, 2019.

(51) Int. Cl. C07D 401/12; C07C 311/19; C07D 307/64

(52) U.S. Cl. CPC C07D 401/12; C07D 307/64; C07C 311/19

(57) ABSTRACT

In one aspect, compounds of Formula AA, or a pharmaceutically acceptable salt thereof, are featured: (Formula AA) or a pharmaceutically acceptable salt thereof, wherein the variables shown in Formula A can be as defined anywhere herein.

The application claims priority to U.S.62/795,969 and U.S.62/795,964, each of which is incorporated herein in their entirety.

### TECHNICAL FIELD

This disclosure features chemical entities (e.g., a compound that modulates (e.g., antagonizes) NLRP3, or a pharmaceutically acceptable salt, and/or hydrate, and/or cocrystal, and/or drug combination of the compound) that are useful, e.g., for treating a condition, disease or disorder in which a decrease or increase in NLRP3 activity (e.g., an increase, e.g., a condition, disease or disorder associated with NLRP3 signaling) contributes to the pathology and/or symptoms and/or progression of the condition, disease or disorder in a subject (e.g., a human). This disclosure also features compositions as well as other methods of using and making the same.

The present disclosure also relates to, in part, methods and compositions for treating anti-TNFα resistance in a subject with an NLRP3 antagonist. The present disclosure also relates, in part, to methods, combinations and compositions for treating TFNα related diseases and anti-TNFα resistance in a subject that include administration of an NLRP3 antagonist, an NLRP3 antagonist and an anti-TNFα agent, or a composition encompassing an NLRP3 antagonist and an anti-TNFα agent.

### BACKGROUND

The NLRP3 inflammasome is a component of the inflammatory process and its aberrant activation is pathogenic in inherited disorders such as the cryopyrin associated periodic syndromes (CAPS). The inherited CAPS Muckle-Wells syndrome (MWS), familial cold autoinflammatory syndrome (FCAS) and neonatal onset multi-system inflammatory disease (NOMID) are examples of indications that have been reported to be associated with gain of function mutations in NLRP3.

NLRP3 can form a complex and has been implicated in the pathogenesis of a number of complex diseases, including but not limited to metabolic disorders such as type 2 diabetes, atherosclerosis, obesity and gout, as well as diseases of the central nervous system, such as Alzheimer's disease and multiple sclerosis and Amyotrophic Lateral Sclerosis and Parkinson disease, lung disease, such as asthma and COPD and pulmonary idiopathic fibrosis, liver disease, such as NASH syndrome, viral hepatitis and cirrhosis, pancreatic disease, such as acute and chronic pancreatitis, kidney disease, such as acute and chronic kidney injury, intestinal disease such as Crohn's disease and Ulcerative Colitis, skin disease such as psoriasis, musculoskeletal disease such as scleroderma, vessel disorders, such as giant cell arteritis, disorders of the bones, such as Osteoarthritis , osteoporosis and osteopetrosis disorders eye disease, such as glaucoma and macular degeneration, diseased caused by viral infection such as HIV and AIDS, autoimmune disease such as Rheumatoid Arthritis, Systemic Lupus Erythematosus, Autoimmune Thyroiditis, Addison's disease, pernicious anemia, cancer and aging.

In light of the above, it would be desirable to provide compounds that modulate (e.g., antagonize) NLRP3.

Additionally, several patients having inflammatory or autoimmune diseases are treated with anti-TNFα agents. A subpopulation of such patients develop resistance to treatment with the anti-TNFα agents. It is desirable to develop methods for reducing a patient's resistance to anti-TNFα agents. In light of the this, it would also be desirable to provide alternative therapies for treating inflammatory or autoimmune diseases (for example NLRP3 inflammasome inhibitors) to avoid or minimise the use of anti-TNFα agents.

Intestinal bowel disease (IBD), encompassing Ulcerative Colitis (UC) and Crohn's disease (CD), are chronic diseases characterized by barrier dysfunction and uncontrolled inflammation and mucosal immune reactions in the gut. A number of inflammatory pathways have been implicated in the progression of IBD, and anti-inflammatory therapy such as tumor necrosis factor-alpha (TNF-α) blockade has shown efficacy in the clinic (Rutgeerts P et al N Engl J Med 2005; 353:2462-76). Anti-TNFa therapies, however, do not show complete efficacy, however, other cytokines such as IL-1β, IL-6, IL-12, IL-18, IL-21, and IL-23 have been shown to drive inflammatory disease pathology in IBD (Neurath MF Nat Rev Immunol 2014; 14; 329-42). IL-1β and IL-18 are produced by the NLRP3 inflammasome in response to pathogenic danger signals, and have been shown to play a role in IBD. Anti-IL-1β therapy is efficacious in patients with IBD driven by genetic mutations in CARD8 or IL-10R (Mao L et al, J Clin Invest 2018; 238:1793-1806, Shouval D S et al, Gastroenterology 2016; 151:1100-1104), IL-18 genetic polymorphisms have been linked to UC (Kanai T et al, Curr Drug Targets 2013; 14:1392-9), and NLRP3 inflammasome inhibitors have been shown to be efficacious in murine models of IBD (Perera A P et al, Sci Rep 2018; 8:8618). Resident gut immune cells isolated from the lamina propria of IBD patients can produce IL-1β, either spontaneously or when stimulated by LPS, and this IL-1β production can be blocked by the ex vivo addition of a NLRP3 antagonist. Based on strong clinical and preclinical evidence showing that inflammasome-driven IL-1β and IL-18 play a role in IBD pathology, it is clear that NLRP3 inflammasome inhibitors could be an efficacious treatment option for UC, Crohn's disease, or subsets of IBD patients. These subsets of patients could be defined by their peripheral or gut levels of inflammasome related cytokines including IL-1β, IL-6, and IL-18, by genetic factors that pre-dispose IBD patients to having NLRP3 inflammasome activation such as mutations in genes including ATG16L1, CARD8, IL-10R, or PTPN2 (Saitoh T et al, Nature 2008; 456:264, Spalinger M R, Cell Rep 2018; 22:1835), or by other clinical rationale such as non-response to TNF therapy.

Though anti-TNF therapy is an effective treatment option for Crohn's disease, 40% of patients fail to respond. One-third of non-responsive CD patients fail to respond to anti-TNF therapy at the onset of treatment, while another third lose response to treatment over time (secondary non-response). Secondary non-response can be due to the generation of anti-drug antibodies, or a change in the immune compartment that desensitizes the patient to anti-TNF (Ben-Horin Set al, Autoimmun Rev 2014; 13:24-30, Steenholdt C et al Gut 2014; 63:919-27). Anti-TNF reduces inflammation in IBD by causing pathogenic T cell apoptosis in the intestine, therefore eliminating the T cell mediated inflammatory response (Van den Brande et al Gut 2007:56:509-17). There is increased NLRP3 expression and increased production of IL-1β in the gut of TNF-non-responsive CD patients (Leal R F et al Gut 2015; 64:233-42) compared to TNF-responsive patients, suggesting NLRP3 inflammasome pathway activation. Furthermore, there is increased expression of TNF-receptor 2 (TNF-R2), which allows for TNF-mediated proliferation of T cells (Schmitt H et al Gut 2018; 0:1-15). IL-1β signaling in the gut promotes T cell differentiation toward Th1/17 cells which can escape anti-TNF-α mediated apoptosis. It is therefore likely that NLRP3 inflammasome activation can cause non-responsiveness in CD patients to anti-TNF-α therapy by sensitizing pathogenic T cells in the gut to anti-TNF-α mediated apoptosis. Experimental data from immune cells isolated from the gut of TNF-resistant Crohn's patients show that these cells spontaneously release IL-1β, which can be inhibited by the addition of an NLRP3 antagonist. NLRP3 inflammasome antagonists—in part by blocking IL-1β secretion—would be expected to inhibit the mechanism leading to anti-TNF non-responsiveness, re-sensitizing the patient to anti-TNF therapy. In IBD patients who are naive to anti-TNF therapy, treatment with an NLRP3 antagonist would be expected to prevent primary- and secondary-non responsiveness by blocking the mechanism leading to non-response.

NLRP3 antagonists that are efficacious locally in the gut can be efficacious drugs to treat IBD; in particular in the treatment of TNF-resistant CD alone or in combination with anti-TNF therapy. Systemic inhibition of both IL-1β and TNF-α has been shown to increase the risk of opportunistic infections (Genovese M C et al, Arthritis Rheum 2004; 50:1412), therefore, only blocking the NLRP3 inflammasome at the site of inflammation would reduce the infection risk inherent in neutralizing both IL-1β and TNF-α. NLRP3 antagonists that are potent in NLRP3-inflammasome driven cytokine secretion assays in cells, but have low permeability in vitro in a permeability assay such as an MDCK assay, have poor systemic bioavailability in a rat or mouse pharmacokinetic experiment, but high levels of compound in the colon and/or small intestine could be a useful therapeutic option for gut restricted purposes.

In light of the above, the present invention also provides alternative therapies for the treatment of inflammatory or autoimmune diseases, including IBD, that solves the above problems associated with anti-TNFα agents.

### SUMMARY

This disclosure features chemical entities (e.g., a compound that modulates (e.g., antagonizes) NLRP3, or a pharmaceutically acceptable salt, and/or hydrate, and/or cocrystal, and/or drug combination of the compound) that are useful, e.g., for treating a condition, disease or disorder in which a decrease or increase in NLRP3 activity (e.g., an increase, e.g., a condition, disease or disorder associated with NLRP3 signaling).

In one aspect, provided herein is a compound of Formula AA

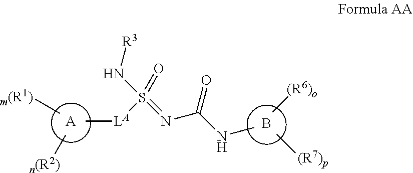

or a pharmaceutically acceptable salt thereof, wherein the variables in Formula AA can be as defined anywhere herein.

In some embodiments, the compound of Formula AA is an unequal mixture (e.g., a non-racemic mixture when the sulfur is the only stereocenter in Formula AA) of:

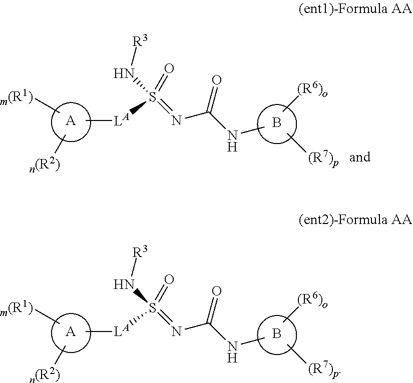

In certain of these embodiments, the mixture is enriched in (ent1)-Formula AA.

In certain of other embodiments, the mixture is enriched in (ent2)-Formula AA.

In certain embodiments, the compound is a compound of (ent1)-Formula AA that is substantially free (e.g., contains less than about 10% of, less than about 5% of, less than about 2% of, less than about 1%, less than about 0.5% of) of (ent2)-Formula AA.

In certain embodiments, the compound is a compound of (ent2)-Formula AA that is substantially free (e.g., contains less than about 10% of, less than about 5% of, less than about 2% of, less than about 1%, less than about 0.5% of) of (ent1)-Formula AA.

In another aspect, provided herein is a compound of Formula AA-CN:

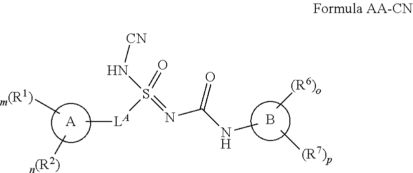

or a pharmaceutically acceptable salt thereof, wherein the variables in Formula AA-CN can be as defined anywhere herein.

In some embodiments, the compound of Formula AA-CN is an unequal mixture (e.g., a non-racemic mixture when the sulfur is the only stereocenter in Formula AA-CN) of:

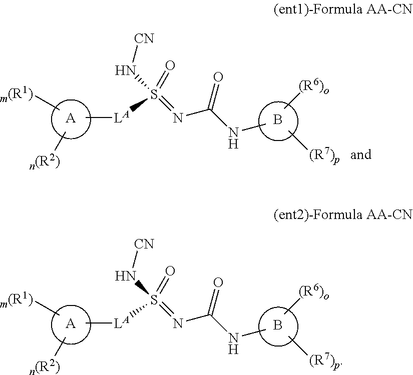

In certain of these embodiments, the mixture is enriched in (ent1)-Formula AA-CN.

In certain of other embodiments, the mixture is enriched in (ent2)-Formula AA-CN.

In certain embodiments, the compound is a compound of (ent1)-Formula AA-CN that is substantially free (e.g., contains less than about 10% of, less than about 5% of, less than about 2% of, less than about 1%, less than about 0.5% of) of (ent2)-Formula AA-CN.

In certain embodiments, the compound is a compound of (ent2)-Formula AA-CN that is substantially free (e.g., contains less than about 10% of, less than about 5% of, less than about 2% of, less than about 1%, less than about 0.5% of) of (ent1)-Formula AA-CN.

The inventors have discovered that in the corresponding N—H (R³═H) compounds, activity primarily resides in only one of the two sulfur enantiomers or epimers; however, surprisingly, when said hydrogen atom is replaced with a cyano group, activity resides primarily in the opposing enantiomer or epimer.

This disclosure also features compositions as well as other methods of using and making the same.

An “antagonist” of NLRP3 includes compounds that inhibit the ability of NLRP3 to induce the production of IL-1β and/or IL-18 by directly binding to NLRP3, or by inactivating, destabilizing, altering distribution, of NLRP3 or otherwise.

In one aspect, pharmaceutical compositions are featured that include a chemical entity described herein (e.g., a compound described generically or specifically herein or a pharmaceutically acceptable salt thereof or compositions containing the same) and one or more pharmaceutically acceptable excipients.

In one aspect, methods for modulating (e.g., agonizing, partially agonizing, antagonizing) NLRP3 activity are featured that include contacting NLRP3 with a chemical entity described herein (e.g., a compound described generically or specifically herein or a pharmaceutically acceptable salt thereof or compositions containing the same). Methods include in vitro methods, e.g., contacting a sample that includes one or more cells comprising NLRP3, as well as in vivo methods.

In a further aspect, methods of treatment of a disease in which NLRP3 signaling contributes to the pathology and/or symptoms and/or progression of the disease are featured that include administering to a subject in need of such treatment an effective amount of a chemical entity described herein (e.g., a compound described generically or specifically herein or a pharmaceutically acceptable salt thereof or compositions containing the same).

In a further aspect, methods of treatment are featured that include administering to a subject a chemical entity described herein (e.g., a compound described generically or specifically herein or a pharmaceutically acceptable salt thereof or compositions containing the same), wherein the chemical entity is administered in an amount effective to treat a disease in which NLRP3 signaling contributes to the pathology and/or symptoms and/or progression of the disease, thereby treating the disease.

The present invention is also relates to the Applicant's discovery that inhibition of NLRP3 inflammasomes can increase a subject's sensitivity to an anti-TNFα agent or can overcome resistance to an anti-TNFα agent in a subject, or indeed provide an alternative therapy to anti-TNFα agents.

Provided herein are methods of treating a subject that include: (a) identifying a subject having a cell that has an elevated level of NLRP3 inflammasome activity and/or expression as compared to a reference level; and (b) administering to the identified subject a therapeutically effective amount of an compound of Formula I or a pharmaceutically acceptable salt, solvate, or co-crystal thereof.

Provided herein are methods for the treatment of inflammatory or autoimmune disease including IBD, such as UC and CD in a subject in need thereof, comprising administering to said subject a therapeutically effective amount a compound for Formula I or a pharmaceutically acceptable salt, solvate, or co-crystal thereof, wherein the NLRP3 antagonist is a gut-targeted NLRP3 antagonist.

Provided herein are methods of treating a subject in need thereof, that include: (a) identifying a subject having resistance to an anti-TNFα agent; and (b) administering a treatment comprising a therapeutically effective amount of a compound for Formula I, or a pharmaceutically acceptable salt, solvate, or co-crystal thereof to the identified subject.

Provided herein are methods of treating a subject in need thereof, that include: administering a treatment comprising a therapeutically effective amount of a compound for Formula I or a pharmaceutically acceptable salt, solvate, or co-crystal thereof to a subject identified as having resistance to an anti-TNFα agent.

Provided herein are methods of selecting a treatment for a subject in need thereof, that include: (a) identifying a subject having resistance to an anti-TNFα agent; and (b) selecting for the identified subject a treatment comprising a therapeutically effective amount of a compound for Formula I or a pharmaceutically acceptable salt, solvate, or co-crystal thereof.

Provided herein are methods of selecting a treatment for a subject in need thereof, that include selecting a treatment comprising a therapeutically effective amount of a compound for Formula I or a pharmaceutically acceptable salt, solvate, or co-crystal thereof for a subject identified as having resistance to an anti-TNFα agent.

In some embodiments of any of the methods described herein, the treatment further includes a therapeutically effective amount of an anti-TNFα agent, in addition to the NLRP3 antagonist.

Embodiments can include one or more of the following features.

The chemical entity can be administered in combination with one or more additional therapies with one or more agents suitable for the treatment of the condition, disease or disorder.

Examples of the indications that may be treated by the compounds disclosed herein include but are not limited to metabolic disorders such as type 2 diabetes, atherosclerosis, obesity and gout, as well as diseases of the central nervous system, such as Alzheimer's disease and multiple sclerosis and Amyotrophic Lateral Sclerosis and Parkinson disease, lung disease, such as asthma and COPD and pulmonary idiopathic fibrosis, liver disease, such as NASH syndrome, viral hepatitis and cirrhosis, pancreatic disease, such as acute and chronic pancreatitis, kidney disease, such as acute and chronic kidney injury, intestinal disease such as Crohn's disease and Ulcerative Colitis, skin disease such as psoriasis, musculoskeletal disease such as scleroderma, vessel disorders, such as giant cell arteritis, disorders of the bones, such as osteoarthritis , osteoporosis and osteopetrosis disorders, eye disease, such as glaucoma and macular degeneration, diseases caused by viral infection such as HIV and AIDS, autoimmune disease such as rheumatoid arthritis, systemic Lupus erythematosus, autoimmune thyroiditis; Addison's disease, pernicious anemia, cancer and aging.

The methods can further include identifying the subject.

Other embodiments include those described in the Detailed Description and/or in the claims.

Additional Definitions

To facilitate understanding of the disclosure set forth herein, a number of additional terms are defined below. Generally, the nomenclature used herein and the laboratory procedures in organic chemistry, medicinal chemistry, and pharmacology described herein are those well-known and commonly employed in the art. Unless defined otherwise, all technical and scientific terms used herein generally have the same meaning as commonly understood by one of ordinary skill in the art to which this disclosure belongs. Each of the patents, applications, published applications, and other publications that are mentioned throughout the specification and the attached appendices are incorporated herein by reference in their entireties.

As used herein, the term “NLRP3” is meant to include, without limitation, nucleic acids, polynucleotides, oligonucleotides, sense and antisense polynucleotide strands, complementary sequences, peptides, polypeptides, proteins, homologous and/or orthologous NLRP3 molecules, isoforms, precursors, mutants, variants, derivatives, splice variants, alleles, different species, and active fragments thereof.

The term “acceptable” with respect to a formulation, composition or ingredient, as used herein, means having no persistent detrimental effect on the general health of the subject being treated.

“API” refers to an active pharmaceutical ingredient.

The terms “effective amount” or “therapeutically effective amount,” as used herein, refer to a sufficient amount of a chemical entity (e.g., a compound exhibiting activity as a modulator of NLRP3, or a pharmaceutically acceptable salt and/or hydrate and/or cocrystal thereof;) being administered which will relieve to some extent one or more of the symptoms of the disease or condition being treated. The result includes reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case is determined using any suitable technique, such as a dose escalation study.

The term “excipient” or “pharmaceutically acceptable excipient” means a pharmaceutically-acceptable material, composition, or vehicle, such as a liquid or solid filler, diluent, carrier, solvent, or encapsulating material. In one embodiment, each component is “ pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, e.g., Remington: The Science and Practice of Pharmacy, 21st ed.; Lippincott Williams & Wilkins: Philadelphia, Pa., 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, Fla., 2009.

The term “pharmaceutically acceptable salt” may refer to pharmaceutically acceptable addition salts prepared from pharmaceutically acceptable non-toxic acids including inorganic and organic acids. In certain instances, pharmaceutically acceptable salts are obtained by reacting a compound described herein, with acids such as hydrochloric acid, hydrobromic acid, sulfuric acid, nitric acid, phosphoric acid, methanesulfonic acid, ethanesulfonic acid, p-toluenesulfonic acid, salicylic acid and the like. The term “pharmaceutically acceptable salt” may also refer to pharmaceutically acceptable addition salts prepared by reacting a compound having an acidic group with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, and salts with amino acids such as arginine, lysine, and the like, or by other methods previously determined. The pharmacologically acceptable salt s not specifically limited as far as it can be used in medicaments. Examples of a salt that the compounds described hereinform with a base include the following: salts thereof with inorganic bases such as sodium, potassium, magnesium, calcium, and aluminum; salts thereof with organic bases such as methylamine, ethylamine and ethanolamine; salts thereof with basic amino acids such as lysine and ornithine; and ammonium salt. The salts may be acid addition salts, which are specifically exemplified by acid addition salts with the following: mineral acids such as hydrochloric acid, hydrobromic acid, hydroiodic acid, sulfuric acid, nitric acid, and phosphoric acid:organic acids such as formic acid, acetic acid, propionic acid, oxalic acid, malonic acid, succinic acid, fumaric acid, maleic acid, lactic acid, malic acid, tartaric acid, citric acid, methanesulfonic acid, and ethanesulfonic acid; acidic amino acids such as aspartic acid and glutamic acid.

The term “pharmaceutical composition” refers to a mixture of a compound described herein with other chemical components (referred to collectively herein as “excipients”), such as carriers, stabilizers, diluents, dispersing agents, suspending agents, and/or thickening agents. The pharmaceutical composition facilitates administration of the compound to an organism. Multiple techniques of administering a compound exist in the art including, but not limited to: rectal, oral, intravenous, aerosol, parenteral, ophthalmic, pulmonary, and topical administration.

The term “subject” refers to an animal, including, but not limited to, a primate (e.g., human), monkey, cow, pig, sheep, goat, horse, dog, cat, rabbit, rat, or mouse. The terms “subject” and “patient” are used interchangeably herein in reference, for example, to a mammalian subject, such as a human.

As used herein, the term “prevent”, “preventing” or “prevention” in connection to a disease or disorder refers to the prophylactic treatment of a subject who is at risk of developing a condition (e.g., specific disease or disorder or clinical symptom thereof) resulting in a decrease in the probability that the subject will develop the condition.

The terms “treat,” “treating,” and “treatment,” in the context of treating a disease or disorder, are meant to include alleviating or abrogating a disorder, disease, or condition, or one or more of the symptoms associated with the disorder, disease, or condition; or to slowing the progression, spread or worsening of a disease, disorder or condition or of one or more symptoms thereof.

The terms “hydrogen” and “H” are used interchangeably herein.

The term “halo” refers to fluoro (F), chloro (Cl), bromo (Br), or iodo (I).

The term “alkyl” refers to a hydrocarbon chain that may be a straight chain or branched chain, saturated or unsaturated, containing the indicated number of carbon atoms. For example, C₁₋₁₀ indicates that the group may have from 1 to 10 (inclusive) carbon atoms in it. Non-limiting examples include methyl, ethyl, iso-propyl, tent-butyl, n-hexyl.

The term “haloalkyl” refers to an alkyl, in which one or more hydrogen atoms is/are replaced with an independently selected halo.

The term “alkoxy” refers to an —O-alkyl radical (e.g., —OCH₃).

The term “carbocyclic ring” as used herein includes an aromatic or nonaromatic cyclic hydrocarbon group having 3 to 10 carbons, such as 3 to 8 carbons, such as 3 to 7 carbons, which may be optionally substituted. Examples of carbocyclic rings include five-membered, six-membered, and seven-membered carbocyclic rings.

The term “heterocyclic ring” refers to an aromatic or nonaromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively), wherein 0, 1, 2, or 3 atoms of each ring may be substituted by a substituent. Each ring of a bicyclic or tricyclic heterocyclic ring is selected from saturated, unsaturated, and aromatic (carbocyclic aromatic and heteroaromatic) rings. Examples of heterocyclic rings include five-membered, six-membered, and seven-membered heterocyclic rings.

The term “cycloalkyl” as used herein includes an nonaromatic cyclic, bicylic, fused, or spiro hydrocarbon radical having 3 to 10 carbons, such as 3 to 8 carbons, such as 3 to 7 carbons, wherein the cycloalkyl group which may be optionally substituted. Examples of cycloalkyls include five-membered, six-membered, and seven-membered rings. Examples include cyclopropyl, cyclobutyl, cyclopentyl, cyclopentenyl, cyclohexyl, cyclohexenyl, cycloheptyl, and cyclooctyl.

The term “heterocycloalkyl” refers to a 5-8 membered monocyclic, 8-12 membered bicyclic, or 11-14 membered tricyclic fused or spiro ring system radical wherein at least one of the rings in the ring system (1) is nonaromatic and (2) includes 1-3 heteroatoms. When the ring system is bicyclic, 1-6 heteroatom ring members are present; and when the ring system is tricyclic, 1-9 heteroatom ring members are present. The ring heteroatoms are selected from O, N, and S (e.g., the ring system includes carbon atoms and 1-3, 1-6, or 1-9 heteroatoms selected from N, O, and S if monocyclic, bicyclic, or tricyclic, respectively), wherein 0, 1, 2, or 3 atoms of each ring may be substituted by a substituent. Examples of heterocycloalkyls include five-membered, six-membered, seven-membered, eight-membered, and ten-membered rings. Examples include piperazinyl, pyrrolidinyl, dioxanyl, morpholinyl, tetrahydrofuranyl, (3aR,6aS)-hexahydro-1H-thieno[3,4-d]imidazole, 1,2,3,4-tetrahydroisoquinoline, and the like.

The term “aryl” is intended to mean an aromatic ring radical containing 6 to 10 ring carbons. Examples include phenyl and naphthyl.

The term “heteroaryl” is intended to mean an aromatic ring system containing 5 to 14 aromatic ring atoms that may be a single ring, two fused rings or three fused rings wherein at least one aromatic ring atom has a heteroatom selected from, but not limited to, the group consisting of O, S and N. Examples include furanyl, thienyl, pyrrolyl, imidazolyl, oxazolyl, thiazolyl, isoxazolyl, pyrazolyl, isothiazolyl, oxadiazolyl, triazolyl, thiadiazolyl, pyridinyl, pyrazinyl, pyrimidinyl, pyridazinyl, triazinyl and the like. Examples also include carbazolyl, quinolizinyl, quinolinyl, isoquinolinyl, cinnolinyl, phthalazinyl, quinazolinyl, quinoxalinyl, triazinyl, indolyl, isoindolyl, indazolyl, indolizinyl, purinyl, naphthyridinyl, pteridinyl, carbazolyl, acridinyl. phenazinyl, phenothiazinyl, phenoxazinyl, benzoxazolyl, benzothiazolyl, 1H-benzimidazolyl, imidazopyridinyl, benzothienyl, benzofuranyl, isobenzofuran and the like.

The term “hydroxy” refers to an OH group.

The term “amino” refers to an NH₂ group.

The term “oxo” refers to O. By way of example, substitution of a CH₂ a group with oxo gives a C═O group.

As used herein, the terms “the ring A” or “A” are used interchangeably to denote

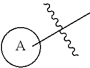

in formula AA, wherein the bond that is shown as being broken by the wavy line [symbol] connects A to the L^{A}-S(O)(NHR³)═N moiety of Formula AA.

As used herein, the terms “the ring B” or “B” are used interchangeably to denote

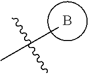

in formula AA wherein the bond that is shown as being broken by the wavy line [symbol] connects B to the NH(CO) group of Formula AA.

As used herein, the term “the optionally substituted ring A” is used to denote

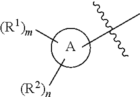

in formula AA, wherein the bond that is shown as being broken by the wavy line [symbol] connects A to the L^{A}-S(O)(NHR³)═N moiety of Formula AA.

As used herein, the term “the substituted ring B” is used to denote

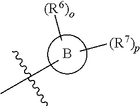

in formula AA, wherein the bond that is shown as being broken by the wavy line [symbol] connects B to the NH(CO) group of Formula AA.

As used herein, the recitation “S(O₂)”, alone or as part of a larger recitation, refers to the group

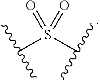 [SMILES: *S(*)(=O)=O]

In addition, atoms making up the compounds of the present embodiments are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include ¹³C and ¹⁴C.

The scope of the compounds disclosed herein includes tautomeric form of the compounds. Thus, by way of example, a compound that is represented as containing the moiety

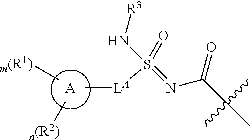

is also intended to include the tautomeric form containing the moiety

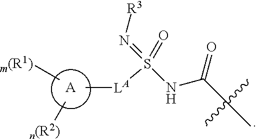

In addition, by way of example, a compound that is represented as containing the moiety

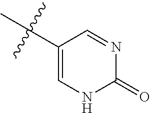 [SMILES: *c1cnc(=O)[nH]c1]

is also intended to include the tautomeric form containing the moiety

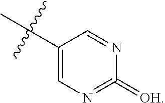

Non-limiting exemplified compounds of the formulae described herein include a stereogenic sulfur atom and optionally one or more stereogenic carbon atoms. This disclosure provides examples of stereoisomer mixtures (e.g., racemic mixture of enantiomers; mixture of diastereomers). This disclosure also describes and exemplifies methods for separating individual components of said stereoisomer mixtures (e.g., resolving the enantiomers of a racemic mixture). In cases of compounds containing only a stereogenic sulfur atom, resolved enantiomers are graphically depicted using one of the two following formats: formulas A/B (hashed and solid wedge three-dimensional representation); and formula C (“flat structures with *-labelled stereogenic sulfur).

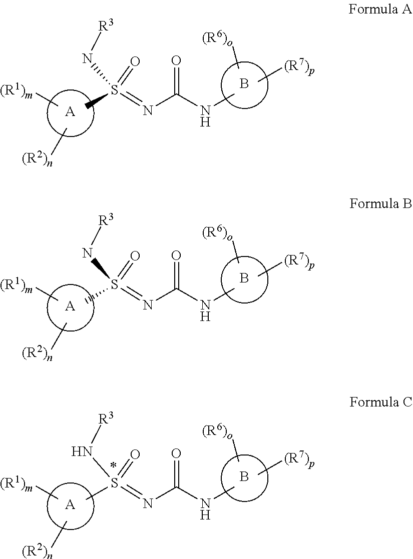

In reaction schemes showing resolution of a racemic mixture, Formulas A/B and C are intended only to convey that the constituent enantiomers were resolved in enantiopure pure form (about 98% ee or greater). The schemes that show resolution products using the formula A/B format are not intended to disclose or imply any correlation between absolute configuration and order of elution. Some of the compounds shown in the tables below are graphically represented using the formula A/B format.

The details of one or more embodiments of the invention are set forth in the description below. Other features and advantages of the invention will be apparent from the description and from the claims.

### DESCRIPTION OF THE DRAWINGS

FIG. 1: Expression levels of RNA encoding NLRP3 in Crohn's Disease patients who are responsive and non-responsive to infliximab.

FIG. 2: Expression levels of RNA encoding IL-1β in Crohn's Disease patients who are responsive and non-responsive to infliximab.

FIG. 3: Expression levels of RNA encoding NLRP3 in Ulcerative Colitis (UC) patients who are responsive and non-responsive to infliximab.

FIG. 4: Expression levels of RNA encoding IL-1β in Ulcerative Colitis (UC) patients who are responsive and non-responsive to infliximab.

### DETAILED DESCRIPTION

In some embodiments, provided herein is a compound of Formula AA

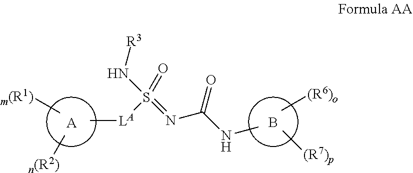

wherein

L^{A} is a bond or a C₁-C₈ alkylene optionally substituted with halo;

m=0, 1, or 2;

n=0, 1, or 2;

o=1 or 2;

p=0, 1, 2, or 3,

wherein

A is a 5- to 10-membered monocyclic or bicyclic heteroaryl or a C₆-C₁₀ monocyclic or bicyclic aryl;

B is a 5-10 membered monocyclic or bicyclic heteroaryl, or a C₆-C₁₀ monocyclic or bicyclic aryl; wherein at least one R⁶ is ortho to the bond connecting the B ring to the NH(CO) group of Formula AA;

R¹ and R² are each independently selected from C₁-C₆ alkyl, C₁-C₆ haloalkyl, C₁-C₆ alkoxy, C₁-C₆ haloalkoxy, halo, CN, NO₂, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NR⁸R⁹, C(O)R¹³, CONR⁸R⁹, SF₅, SC₁-C₆ alkyl, S(O₂)C₁-C₆ alkyl, S(O)C₁-C₆ alkyl, S(O₂)NR¹¹R¹², C₃-C₇ cycloalkyl, and 3- to 7-membered heterocycloalkyl,

wherein the C₁-C₆ alkyl, C₁-C₆ alkoxy, C₁-C₆ haloalkyl, C₆-C₁₀ aryl, 5- to 10-membered heteroaryl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, R¹⁵, C₁-C₆ haloalkoxy, NR⁸R⁹, ═NR¹⁰, COOC₁-C₆ alkyl, CONR⁸R⁹, 3- to 7-membered heterocycloalkyl, C₃-C₇ cycloalkyl, C₆-C₁₀ aryl, 5- to 10-membered heteroaryl, OCOC₁-C₆ alkyl, OCOC₆-C₁₀ aryl, OCO(5- to 10-membered heteroaryl), and OCO(3- to 7-membered heterocycloalkyl);

wherein each C₁-C₆ alkyl substituent and each C₁-C₆ alkoxy substituent of the R¹ or R² C₃-C₇ cycloalkyl or of the R¹ or R² 3- to 7-membered heterocycloalkyl is further optionally independently substituted with one to three hydroxy, —O(C₀-C₃ alkylene)C₆-C₁₀ aryl, halo, NR⁸R⁹, or oxo;

wherein the 3- to 7-membered heterocycloalkyl, C₆-C₁₀ aryl, and 5- to 10-membered heteroaryl are optionally substituted with one or more substituents independently selected from halo, C₁-C₆ alkyl, and OC₁-C₆ alkyl;

or one pair of R¹ and R² on adjacent atoms, taken together with the atoms connecting them, independently form one monocyclic or bicyclic C₄-C₁₂ carbocyclic ring or one monocyclic or bicyclic 5- to-12-membered heterocyclic ring containing 1-3 heteroatoms independently selected from O, N, and S, wherein the carbocyclic ring or heterocyclic ring is optionally independently substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR⁸R⁹, ═NR¹⁰, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR⁸R⁹,

wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR⁸R⁹, ═NR¹⁰, COOC₁-C₆ alkyl, C₆-C₁₀ aryl, and CONR⁸R⁹;

R⁶ and R⁷ are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, C₃-C₁₀ cycloalkyl, 3- to 10-membered heterocycloalkyl, and C₂-C₆ alkenyl,

wherein R⁶ and R⁷ are each optionally substituted with one or more substituents independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), C₆-C₁₀ aryloxy, and S(O₂)C₁-C₆ alkyl; and wherein the C₁-C₆ alkyl or C₁-C₆ alkoxy that R⁶ or R⁷ is substituted with is optionally substituted with one or more hydroxyl, C₆-C₁₀ aryl or NR⁸R⁹, or wherein R⁶ or R⁷ is optionally fused to a five- to -seven-membered carbocyclic ring or heterocyclic ring containing one or two heteroatoms independently selected from oxygen, sulfur and nitrogen;

wherein the 3- to 7-membered heterocycloalkyl, C₆-C₁₀ aryl, 5- to 10-membered heteroaryl are optionally substituted with one or more substituents independently selected from halo, C₁-C₆ alkyl, and OC₁-C₆ alkyl;

or at least one pair of R⁶ and R⁷ on adjacent atoms, taken together with the atoms connecting them, independently form at least one C₄-C₈ carbocyclic ring or at least one 5- to 8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹;

R¹⁰ is C₁-C₆ alkyl;

each of R⁸ and R⁹ at each occurrence is independently selected from hydrogen, C₁-C₆ alkyl, C₂-C₆ alkenyl, C₃-C₇ cycloalkyl, (C═NR¹³)NR¹¹R¹², S(O₂)C₁-C₆ alkyl, S(O₂)NR¹¹R¹², COR¹³, CO₂R¹³ and CONR¹¹R¹²; wherein the C₁-C₆ alkyl is optionally substituted with one or more hydroxy, halo, C₁-C₆ alkoxy, C₆-C₁₀ aryl, 5- to 10-membered heteroaryl, C₃-C₇ cycloalkyl, 3- to 7-membered heterocycloalkyl, or NR¹¹R¹²;

or R⁸ and R⁹ taken together with the nitrogen they are attached to form a 3- to 10-membered monocyclic or bicyclic ring optionally containing one or more heteroatoms in addition to the nitrogen they are attached to, wherein the ring is optionally substituted with one or more substituents independently selected from halo, C₁-C₆ alkyl, C₁-C₆ haloalkyl, C₁-C₆ alkoxy, oxo, N(C₁-C₆alkyl)₂, NH₂, NH(C₁-C₆ alkyl), and hydroxy;

R¹³ is C₁-C₆ alkyl, C₁-C₆ haloalkyl, or —(Z¹-Z²)ₐ₁-Z³;

each of R¹¹ and R¹² at each occurrence is independently selected from hydrogen, C₁-C₆ alkyl, and —(Z¹-Z²)ₐ₁-Z³;

a1 is 0-10 (e.g., 0-4);

each Z¹ is independently C₁-C₆ alkylene optionally substituted with one or more substituents independently selected from oxo, halo, and hydroxy;

each Z² is independently a bond, NH, N(C₁-C₆ alkyl), —O—, —S—, or 5-10 membered heteroarylene;

Z³ is independently C₆-C₁₀ aryl, C₂-C₆ alkenyl, C₂-C₆ alkynyl, C₃-C₁₀ cycloalkyl, 5- to 10-membered heteroaryl, or 3- to 10-membered heterocycloalkyl, each of which is optionally substituted with one or more substituents independently selected from halo, C₁-C₆ alkyl, C₁₋₆ haloalkyl, C₁-C₆ alkoxy, oxo, N(C₁-C₆ alkyl)₂, NH₂, NH(C₁-C₆ alkyl), and hydroxy;

R³ is selected from cyano, hydroxy, C₁-C₆ alkoxy, C(O)OR′, C(O)OR¹⁴, C(O)R′, C(O)NR′R″, C(O)NHR¹⁴, S(O)₀₋₂R′, S(O)₀₋₂R¹⁴, S(O)₁₋₂NR′R″, S(O)₁₋₂NHR¹⁴, and R¹⁴—(C₀-C₂ alkylene)-, wherein the C₀-C₂ alkylene group is optionally substituted with one or more oxo;

R¹⁴ is C₃-C₈ cycloalkyl, 3- to 8-membered heterocycloalkyl, C₂-C₁₀ alkenyl, C₂-C₁₀ alkynyl, 5- to 10-membered monocyclic or bicyclic heteroaryl or C₆-C₁₀ monocyclic or bicyclic aryl, wherein each cycloalkyl, heterocycloalkyl, C₂-C₁₀ alkenyl, C₂-C₁₀ alkynyl, aryl, or heteroaryl is optionally independently substituted with 1 or 2 R⁶;

each of R′ and R″ is independently hydrogen or C₁-C₆ alkyl;

R¹⁵ is —(Z⁴-Z⁵)ₐ₂-Z⁶;

a2 is an integer selected from 1-10 (e.g., 1-5);

each Z⁴ is independently selected from —O—, —S—, —NH—, and —N(C₁-C₃ alkyl)-;

provided that the Z⁴ group directly attached to R¹ or R² is —O— or —S—;

each Z⁵ is independently C₁-C₆ alkylene optionally substituted with one or more substituents independently selected from oxo, halo, and hydroxy; and

Z⁶ is OH, OC₁-C₆ alkyl, NH₂, NH(C₁-C₆ alkyl), N(C₁-C₆ alkyl)₂, NHC(O)(C₁-C₆ alkyl), NHC(O)(C₁-C₆ alkoxy), or an optionally substituted group selected from the group consisting of:

C₆-C₁₀ aryl, C₂-C₆ alkenyl, C₂-C₆ alkynyl, C₃-C₁₀ cycloalkyl, 5- to 10-membered heteroaryl, or 3- to 10-membered heterocycloalkyl, each of which is optionally substituted with one or more substituents independently selected from halo, C₁-C₆ alkyl, C₁₋₆ haloalkyl, C₁-C₆ alkoxy, oxo, N(C₁-C₆ alkyl)₂, NH₂, NH(C₁-C₆ alkyl), and hydroxy;

or a pharmaceutically acceptable salt thereof.

In certain of the foregoing embodiments, it is provided that the compound is other than one or more compounds disclosed in WO 2018225018, which is incorporated herein by reference in its entirety; and

the compound is other than one or more compounds disclosed in IN 201721020305, which is incorporated herein by reference in its entirety.

In some embodiments, provided herein is a compound of Formula AA:

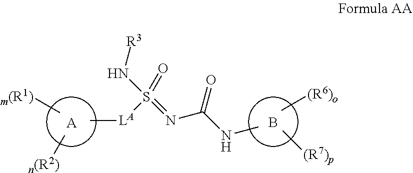

wherein:
the

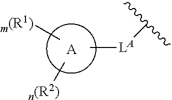

moiety is as defined according to (AA-1), (AA-2), (AA-3), or (AA-4) below:

### (AA-1)

L^{A} is a bond;

m=0, 1, or 2;

n=0, 1, or 2, wherein m+n≥2; and

A is a 5- to 10-membered monocyclic or bicyclic heteroaryl wherein one ring atom is a heteroatom selected from O, N, and S; OR

### (AA-2)

L^{A} is a bond;

m=0, 1, or 2;

n=0, 1, or 2; and

A is a 5- to 10-membered monocyclic or bicyclic heteroaryl wherein from 2-3 ring atoms are heteroatoms, each independently selected from O, N, and S; or a C₇-C₁₀ bicyclic aryl; OR

### (AA-3)

L^{A} is a C₁-C₈ alkylene optionally substituted with halo;

m=0, 1, or 2;

n=0, 1, or 2; and

A is a 5- to 10-membered monocyclic or bicyclic heteroaryl wherein one ring atom is a heteroatom selected from O, N, and S; or a C₇-C₁₀ bicyclic aryl;

### (AA-4)

L^{A} is a C₁-C₈ alkylene optionally substituted with halo;

m=0, 1, or 2; and

n=0, 1, or 2; wherein m+n>2;

A is phenyl;

B is a 5-10 membered monocyclic or bicyclic heteroaryl, or a C₆-C₁₀ monocyclic or bicyclic aryl; wherein at least one R⁶ is ortho to the bond connecting the B ring to the -L-NH(CO) group of Formula AA;

wherein when (AA-1), (AA-2), (AA-3), or (AA-4) applies:

R¹ and R² are each independently selected from C₁-C₆ alkyl, C₁-C₆ haloalkyl, C₁-C₆ alkoxy, C₁-C₆ haloalkoxy, halo, CN, NO₂, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NR⁸R⁹, C(O)R¹³, CONR⁸R⁹, SF₅, SC₁-C₆ alkyl, S(O₂)C₁-C₆ alkyl, S(O)C₁-C₆ alkyl, S(O₂)NR¹¹R¹², C₃-C₇ cycloalkyl, and 3- to 7-membered heterocycloalkyl,

wherein the C₁-C₆ alkyl, C₁-C₆ alkoxy, C₁-C₆ haloalkyl, C₆-C₁₀ aryl, 5- to 10-membered heteroaryl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, 10⁵, C₁-C₆ haloalkoxy, NR⁸R⁹, ═NR¹⁰, COOC₁-C₆ alkyl, CONR⁸R⁹, 3- to 7-membered heterocycloalkyl, C₃-C₇ cycloalkyl, C₆-C₁₀ aryl, 5- to 10-membered heteroaryl, OCOC₁-C₆ alkyl, OCOC₆-C₁₀ aryl, OCO(5- to 10-membered heteroaryl), and OCO(3- to 7-membered heterocycloalkyl);

wherein each C₁-C₆ alkyl substituent and each C₁-C₆ alkoxy substituent of the R¹ or R² C₃-C₇ cycloalkyl or of the R¹ or R² 3- to 7-membered heterocycloalkyl is further optionally independently substituted with one to three hydroxy, —O(C₀-C₃ alkylene)C₆-C₁₀ aryl, halo, NR⁸R⁹, or oxo;

wherein the 3- to 7-membered heterocycloalkyl, C₆-C₁₀ aryl, and 5- to 10-membered heteroaryl are optionally substituted with one or more substituents independently selected from halo, C₁-C₆ alkyl, and OC₁-C₆ alkyl;

or one pair of R¹ and R² on adjacent atoms, taken together with the atoms connecting them, independently form one monocyclic or bicyclic C₄-C₁₂ carbocyclic ring or one monocyclic or bicyclic 5- to-12-membered heterocyclic ring containing 1-3 heteroatoms independently selected from O, N, and S, wherein the carbocyclic ring or heterocyclic ring is optionally independently substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR⁸R⁹, ═NR¹⁰, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR⁸R⁹,

wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR⁸R⁹, ═NR¹⁰, COOC₁-C₆ alkyl, C₆-C₁₀ aryl, and CONR⁸R⁹;

R⁶ and R⁷ are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, C₃-C₁₀ cycloalkyl, 3- to 10-membered heterocycloalkyl, and C₂-C₆ alkenyl,

wherein R⁶ and R⁷ are each optionally substituted with one or more substituents independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), C₆-C₁₀ aryloxy, and S(O₂)C₁-C₆ alkyl; and wherein the C₁-C₆ alkyl or C₁-C₆ alkoxy that R⁶ or R⁷ is substituted with is optionally substituted with one or more hydroxyl, C₆-C₁₀ aryl or NR⁸R⁹, or wherein R⁶ or R⁷ is optionally fused to a five- to -seven-membered carbocyclic ring or heterocyclic ring containing one or two heteroatoms independently selected from oxygen, sulfur and nitrogen;

wherein the 3- to 7-membered heterocycloalkyl, C₆-C₁₀ aryl, 5- to 10-membered heteroaryl are optionally substituted with one or more substituents independently selected from halo, C₁-C₆ alkyl, and OC₁-C₆ alkyl;

or at least one pair of R⁶ and R⁷ on adjacent atoms, taken together with the atoms connecting them, independently form at least one C₄-C₈ carbocyclic ring or at least one 5- to 8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹;

R¹⁰ is C₁-C₆ alkyl;

each of R⁸ and R⁹ at each occurrence is independently selected from hydrogen, C₁-C₆ alkyl, C₂-C₆ alkenyl, C₃-C₇ cycloalkyl, (C═NR¹³)NR¹¹R¹², S(O₂)C₁-C₆ alkyl, S(O₂)NR¹¹R¹², COR¹³, CO₂R¹³ and CONR¹¹R¹²; wherein the C₁-C₆ alkyl is optionally substituted with one or more hydroxy, halo, C₁-C₆ alkoxy, C₆-C₁₀ aryl, 5- to 10-membered heteroaryl, C₃-C₇ cycloalkyl, 3- to 7-membered heterocycloalkyl, or NR¹¹R¹²;

or R⁸ and R⁹ taken together with the nitrogen they are attached to form a 3- to 10-membered monocyclic or bicyclic ring optionally containing one or more heteroatoms in addition to the nitrogen they are attached to, wherein the ring is optionally substituted with one or more substituents independently selected from halo, C₁-C₆ alkyl, C₁-C₆ haloalkyl, C₁-C₆ alkoxy, oxo, N(C₁-C₆ alkyl)₂, NH₂, NH(C₁-C₆ alkyl), and hydroxy;

R¹³ is C₁-C₆ alkyl, C₁-C₆ haloalkyl, or —(Z¹-Z²)ₐ₁-Z³;

each of R¹¹ and R¹² at each occurrence is independently selected from hydrogen, C₁-C₆ alkyl, and —(Z¹-Z²)ₐ₁-Z³;

a1 is 0-10 (e.g., 0-4);

each Z¹ is independently C₁-C₆ alkylene optionally substituted with one or more substituents independently selected from oxo, halo, and hydroxy;

each Z² is independently a bond, NH, N(C₁-C₆ alkyl), —O—, —S—, or 5-10 membered heteroarylene;

Z³ is independently C₆-C₁₀ aryl, C₂-C₆ alkenyl, C₂-C₆ alkynyl, C₃-C₁₀ cycloalkyl, 5- to 10-membered heteroaryl, or 3- to 10-membered heterocycloalkyl, each of which is optionally substituted with one or more substituents independently selected from halo, C₁-C₆ alkyl, C₁₋₆ haloalkyl, C₁-C₆ alkoxy, oxo, N(C₁-C₆alkyl)₂, NH₂, NH(C₁-C₆ alkyl), and hydroxy;

R³ is selected from cyano, hydroxy, C₁-C₆ alkoxy, C(O)OR′, C(O)OR¹⁴, C(O)R′, C(O)NR′R″, C(O)NHR¹⁴, S(O)₀₋₂R¹⁴, S(O)₀₋₂R¹⁴, S(O)₁₋₂NR′R″, S(O)₁₋₂NHR¹⁴, and R¹⁴—(C₀-C₂ alkylene)-, wherein the C₀-C₂ alkylene group is optionally substituted with one or more oxo;

R¹⁴ is C₃-C₈ cycloalkyl, 3- to 8-membered heterocycloalkyl, C₂-C₁₀ alkenyl, C₂-C₁₀ alkynyl, 5- to 10-membered monocyclic or bicyclic heteroaryl or C₆-C₁₀ monocyclic or bicyclic aryl, wherein each cycloalkyl, heterocycloalkyl, C₂-C₁₀ alkenyl, C₂-C₁₀ alkynyl, aryl or heteroaryl is optionally independently substituted with 1 or 2 R⁶;

each of R′ and R″ is independently hydrogen or C₁-C₆ alkyl;

R¹⁵ is —(Z⁴-Z⁵)ₐ₂-Z⁶;

a2 is an integer selected from 1-10 (e.g., 1-5);

each Z⁴ is independently selected from —O—, —S—, —NH—, and —N(C₁-C₃ alkyl)-;

provided that the Z⁴ group directly attached to R¹ or R² is —O— or —S—;

each Z⁵ is independently C₁-C₆ alkylene optionally substituted with one or more substituents independently selected from oxo, halo, and hydroxy; and

Z⁶ is OH, OC₁-C₆ alkyl, NH₂, NH(C₁-C₆ alkyl), N(C₁-C₆ alkyl)₂, NHC(O)(C₁-C₆ alkyl), NHC(O)(C₁-C₆ alkoxy), or an optionally substituted group selected from the group consisting of:

C₆-C₁₀ aryl, C₂-C₆ alkenyl, C₂-C₆ alkynyl, C₃-C₁₀ cycloalkyl, 5- to 10-membered heteroaryl, or 3- to 10-membered heterocycloalkyl, each of which is optionally substituted with one or more substituents independently selected from halo, C₁-C₆ alkyl, C₁₋₆ haloalkyl, C₁-C₆ alkoxy, oxo, N(C₁-C₆alkyl)₂, NH₂, NH(C₁-C₆ alkyl), and hydroxy;

or a pharmaceutically acceptable salt thereof.

In some embodiments, provided herein is a compound of Formula AA

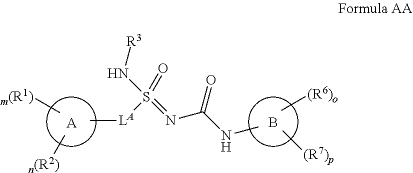

wherein

L^{A} is a bond or a C₁-C₈ alkylene optionally substituted with halo;

m=0, 1, or 2;

n=0, 1, or 2;

wherein

A is a 5- to 10-membered monocyclic or bicyclic heteroaryl or a C₆-C₁₀ monocyclic or bicyclic aryl;

R¹ and R² are each independently selected from C₁-C₆ alkyl, C₁-C₆ haloalkyl, C₁-C₆ alkoxy, C₁-C₆ haloalkoxy, halo, CN, NO₂, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NR⁸R⁹, C(O)R¹³, CONR⁸R⁹, SF₅, SC₁-C₆ alkyl, S(O₂)C₁-C₆ alkyl, S(O)C₁-C₆ alkyl, S(O₂)NR¹¹R¹², C₃-C₇ cycloalkyl, and 3- to 7-membered heterocycloalkyl,

wherein the C₁-C₆ alkyl, C₁-C₆ alkoxy, C₁-C₆ haloalkyl, C₆-C₁₀ aryl, 5- to 10-membered heteroaryl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, R¹⁵, C₁-C₆ haloalkoxy, NR⁸R⁹, ═NR¹⁰, COOC₁-C₆ alkyl, CONR⁸R⁹, 3- to 7-membered heterocycloalkyl, C₃-C₇ cycloalkyl, C₆-C₁₀ aryl, 5- to 10-membered heteroaryl, OCOC₁-C₆ alkyl, OCOC₆-C₁₀ aryl, OCO(5- to 10-membered heteroaryl), and OCO(3- to 7-membered heterocycloalkyl);

wherein each C₁-C₆ alkyl substituent and each C₁-C₆ alkoxy substituent of the R¹ or R² C₃-C₇ cycloalkyl or of the R¹ or R² 3- to 7-membered heterocycloalkyl is further optionally independently substituted with one to three hydroxy, —O(C₀-C₃ alkylene)C₆-C₁₀ aryl, halo, NR⁸R⁹, or oxo;

wherein the 3- to 7-membered heterocycloalkyl, C₆-C₁₀ aryl, and 5- to 10-membered heteroaryl are optionally substituted with one or more substituents independently selected from halo, C₁-C₆ alkyl, and OC₁-C₆ alkyl;

or one pair of R¹ and R² on adjacent atoms, taken together with the atoms connecting them, independently form one monocyclic or bicyclic C₄-C₁₂ carbocyclic ring or one monocyclic or bicyclic 5- to-12-membered heterocyclic ring containing 1-3 heteroatoms independently selected from O, N, and S, wherein the carbocyclic ring or heterocyclic ring is optionally independently substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR⁸R⁹, ═NR¹⁰, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR⁸R⁹,

wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR⁸R⁹, ═NR¹⁰, COOC₁-C₆ alkyl, C₆-C₁₀ aryl, and CONR⁸R⁹;

wherein the

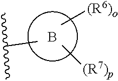

moiety is as defined in (BB-1) or (BB-2) below:

(BB-1)

o=1 or 2;

p=0, 1, 2, or 3,

B is a 5-10 membered monocyclic or bicyclic heteroaryl; wherein at least one R⁶ is ortho to the bond connecting the B ring to the NH(CO) group of Formula AA;

(BB-2)

o=1 or 2;

p=0, 1, 2, or 3,

provided that o+p>2;

B is a C₆-C₁₀ monocyclic or bicyclic aryl; wherein at least one R⁶ is ortho to the bond connecting the B ring to the -L-NH(CO) group of Formula AA;

when (BB-1) or (BB-2) applies:

R⁶ and R⁷ are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, C₃-C₁₀ cycloalkyl, 3- to 10-membered heterocycloalkyl, and C₂-C₆ alkenyl,
wherein R⁶ and R⁷ are each optionally substituted with one or more substituents independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), C₆-C₁₀ aryloxy, and S(O₂)C₁-C₆ alkyl; and wherein the C₁-C₆ alkyl or C₁-C₆ alkoxy that R⁶ or R⁷ is substituted with is optionally substituted with one or more hydroxyl, C₆-C₁₀ aryl or NR⁸R⁹, or wherein R⁶ or R⁷ is optionally fused to a five- to -seven-membered carbocyclic ring or heterocyclic ring containing one or two heteroatoms independently selected from oxygen, sulfur and nitrogen;

wherein the 3- to 7-membered heterocycloalkyl, C₆-C₁₀ aryl, 5- to 10-membered heteroaryl are optionally substituted with one or more substituents independently selected from halo, C₁-C₆ alkyl, and OC₁-C₆ alkyl;

or at least one pair of R⁶ and R⁷ on adjacent atoms, taken together with the atoms connecting them, independently form at least one C₄-C₈ carbocyclic ring or at least one 5- to 8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹; OR

provided that:

when (BB-2) applies; o+p=4; two pairs of R⁶ and R⁷ are on adjacent atoms; and

each pair taken together with the atoms connecting them, independently forms a ring selected from a C₄-C₈ carbocyclic ring and a 5- to 8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹, then at least one ring is selected from:

(a) a C₄ carbocyclic ring, a C₆-C₈ carbocyclic ring, or a 5- to 8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹; and

(b) a C₅ carbocyclic ring substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹;

R¹⁰ is C₁-C₆ alkyl;

each of R⁸ and R⁹ at each occurrence is independently selected from hydrogen, C₁-C₆ alkyl, C₂-C₆ alkenyl, C₃-C₇ cycloalkyl, (C═NR¹³)NR¹¹R¹², S(O₂)C₁-C₆ alkyl, S(O₂)NR¹¹R¹², COR¹³, CO₂R¹³ and CONR¹¹R¹²; wherein the C₁-C₆ alkyl is optionally substituted with one or more hydroxy, halo, C₁-C₆ alkoxy, C₆-C₁₀ aryl, 5- to 10-membered heteroaryl, C₃-C₇ cycloalkyl, 3- to 7-membered heterocycloalkyl, or NR¹¹R¹²;

or R⁸ and R⁹ taken together with the nitrogen they are attached to form a 3- to 10-membered monocyclic or bicyclic ring optionally containing one or more heteroatoms in addition to the nitrogen they are attached to, wherein the ring is optionally substituted with one or more substituents independently selected from halo, C₁-C₆ alkyl, C₁-C₆ haloalkyl, C₁-C₆ alkoxy, oxo, N(C₁-C₆alkyl)₂, NH₂, NH(C₁-C₆ alkyl), and hydroxy;

R¹³ is C₁-C₆ alkyl, C₁-C₆ haloalkyl, or —(Z¹-Z²)ₐ₁-Z³;

each of R¹¹ and R¹² at each occurrence is independently selected from hydrogen, C₁-C₆ alkyl, and —(Z¹-Z²)ₐ₁-Z³;

a1 is 0-10 (e.g., 0-4);

each Z¹ is independently C₁-C₆ alkylene optionally substituted with one or more substituents independently selected from oxo, halo, and hydroxy;

each Z² is independently a bond, NH, N(C₁-C₆ alkyl), —O—, —S—, or 5-10 membered heteroarylene;

Z³ is independently C₆-C₁₀ aryl, C₂-C₆ alkenyl, C₂-C₆ alkynyl, C₃-C₁₀ cycloalkyl, 5- to 10-membered heteroaryl, or 3- to 10-membered heterocycloalkyl, each of which is optionally substituted with one or more substituents independently selected from halo, C₁-C₆ alkyl, C₁₋₆ haloalkyl, C₁-C₆ alkoxy, oxo, N(C₁-C₆alkyl)₂, NH₂, NH(C₁-C₆ alkyl), and hydroxy;

R³ is selected from cyano, hydroxy, C₁-C₆ alkoxy, C(O)OR′, C(O)OR¹⁴, C(O)R′, C(O)NR′R″, C(O)NHR¹⁴, S(O)₀₋₂R′, S(O)₀₋₂R¹⁴, S(O)₁₋₂NR′R″, S(O)₁₋₂NHR¹⁴, and R¹⁴—(C₀-C₂ alkylene)-, wherein the C₀-C₂ alkylene group is optionally substituted with one or more oxo;

R¹⁴ is C₃-C₈ cycloalkyl, 3- to 8-membered heterocycloalkyl, C₂-C₁₀ alkenyl, C₂-C₁₀ alkynyl, 5- to 10-membered monocyclic or bicyclic heteroaryl or C₆-C₁₀ monocyclic or bicyclic aryl, wherein each cycloalkyl, heterocycloalkyl, C₂-C₁₀ alkenyl, C₂-C₁₀ alkynyl, aryl or heteroaryl is optionally independently substituted with 1 or 2 R⁶;

each of R′ and R″ is independently hydrogen or C₁-C₆ alkyl;

R¹⁵ is —(Z⁴-Z⁵)ₐ₂-Z⁶;

a2 is an integer selected from 1-10 (e.g., 1-5);

each Z⁴ is independently selected from —O—, —S—, —NH—, and N(C₁-C₃ alkyl)-;

provided that the Z⁴ group directly attached to R¹ or R² is —O— or —S—;

each Z⁵ is independently C₁-C₆ alkylene optionally substituted with one or more substituents independently selected from oxo, halo, and hydroxy; and

Z⁶ is OH, OC₁-C₆ alkyl, NH₂, NH(C₁-C₆ alkyl), N(C₁-C₆ alkyl)₂, NHC(O)(C₁-C₆ alkyl), NHC(O)(C₁-C₆ alkoxy), or an optionally substituted group selected from the group consisting of:

C₆-C₁₀ aryl, C₂-C₆ alkenyl, C₂-C₆ alkynyl, C₃-C₁₀ cycloalkyl, 5- to 10-membered heteroaryl, or 3- to 10-membered heterocycloalkyl, each of which is optionally substituted with one or more substituents independently selected from halo, C₁-C₆ alkyl, C₁₋₆ haloalkyl, C₁-C₆ alkoxy, oxo, N(C₁-C₆ alkyl)₂, NH₂, NH(C₁-C₆ alkyl), and hydroxy; or a pharmaceutically acceptable salt thereof.

In another aspect, provided herein is a compound of Formula AA-CN:

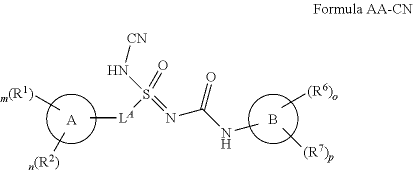

or a pharmaceutically acceptable salt thereof, wherein the variables in Formula AA-CN can be as defined anywhere herein.

In another aspect, provided herein is compound of Formula AA:

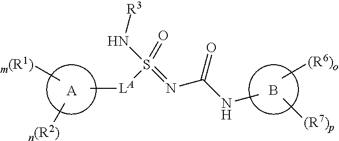

wherein the compound is an unequal mixture (e.g., a non-racemic mixture when the sulfur is the only stereocenter in Formula AA) of:

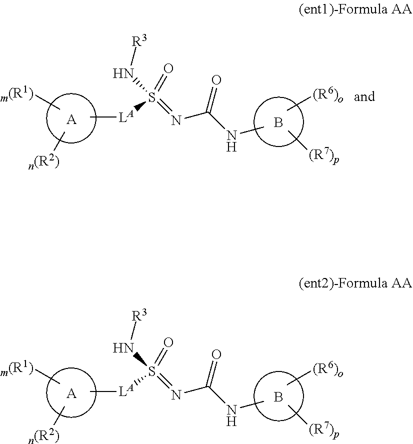

wherein

L^{A} is a bond or a C₁-C₈ alkylene optionally substituted with halo;

m=0, I, or 2;

n=0, 1, or 2;

o=1 or 2;

p=0, 1, 2, or 3,

wherein

A is a 5- to 10-membered monocyclic or bicyclic heteroaryl or a C₆-C₁₀ monocyclic or bicyclic aryl;

B is a 5-10 membered monocyclic or bicyclic heteroaryl, or a C₆-C₁₀ monocyclic or bicyclic aryl; wherein at least one R⁶ is ortho to the bond connecting the B ring to the NH(CO) group of Formula AA;

R¹ and R² are each independently selected from C₁-C₆ alkyl, C₁-C₆ haloalkyl, C₁-C₆ alkoxy, C₁-C₆ haloalkoxy, halo, CN, NO₂, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NR⁸R⁹, C(O)R¹³, CONR⁸R⁹, SF₅, SC₁-C₆ alkyl, S(O₂)C₁-C₆ alkyl, S(O)C₁-C₆ alkyl, S(O₂)NR¹¹R¹², C₃-C₇ cycloalkyl, and 3- to 7-membered heterocycloalkyl,

wherein the C₁-C₆ alkyl, C₁-C₆ alkoxy, C₁-C₆ haloalkyl, C₆-C₁₀ aryl, 5- to 10-membered heteroaryl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, R¹⁵, C₁-C₆ haloalkoxy, NR⁸R⁹, ═NR¹⁰, COOC₁-C₆ alkyl, CONR⁸R⁹, 3- to 7-membered heterocycloalkyl, C₃-C₇ cycloalkyl, C₆-C₁₀ aryl, 5- to 10-membered heteroaryl, OCOC₁-C₆ alkyl, OCOC₆-C₁₀ aryl, OCO(5- to 10-membered heteroaryl), and OCO(3- to 7-membered heterocycloalkyl);

wherein each C₁-C₆ alkyl substituent and each C₁-C₆ alkoxy substituent of the R¹ or R² C₃-C₇ cycloalkyl or of the R¹ or R² 3- to 7-membered heterocycloalkyl is further optionally independently substituted with one to three hydroxy, —O(C₀-C₃ alkylene)C₆-C₁₀ aryl, halo, NR⁸R⁹, or oxo;

wherein the 3- to 7-membered heterocycloalkyl, C₆-C₁₀ aryl, and 5- to 10-membered heteroaryl are optionally substituted with one or more substituents independently selected from halo, C₁-C₆ alkyl, and OC₁-C₆ alkyl;

or one pair of R¹ and R² on adjacent atoms, taken together with the atoms connecting them, independently form one monocyclic or bicyclic C₄-C₁₂ carbocyclic ring or one monocyclic or bicyclic 5- to-12-membered heterocyclic ring containing 1-3 heteroatoms independently selected from O, N, and S, wherein the carbocyclic ring or heterocyclic ring is optionally independently substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR⁸R⁹, ═NR¹⁰, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR⁸R⁹,

wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR⁸R⁹, ═NR¹⁰, COOC₁-C₆ alkyl, C₆-C₁₀ aryl, and CONR⁸R⁹;

R⁶ and R⁷ are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, C₃-C₁₀ cycloalkyl, 3- to 10-membered heterocycloalkyl, and C₂-C₆ alkenyl,

wherein R⁶ and R⁷ are each optionally substituted with one or more substituents independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), C₆-C₁₀ aryloxy, and S(O₂)C₁-C₆ alkyl; and wherein the C₁-C₆ alkyl or C₁-C₆ alkoxy that R⁶ or R⁷ is substituted with is optionally substituted with one or more hydroxyl, C₆-C₁₀ aryl or NR⁸R⁹, or wherein R⁶ or R⁷ is optionally fused to a five- to -seven-membered carbocyclic ring or heterocyclic ring containing one or two heteroatoms independently selected from oxygen, sulfur and nitrogen;

wherein the 3- to 7-membered heterocycloalkyl, C₆-C₁₀ aryl, 5- to 10-membered heteroaryl are optionally substituted with one or more substituents independently selected from halo, C₁-C₆ alkyl, and OC₁-C₆ alkyl;

or at least one pair of R⁶ and R⁷ on adjacent atoms, taken together with the atoms connecting them, independently form at least one C₄-C₈ carbocyclic ring or at least one 5- to 8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹;

R¹⁰ is C₁-C₆ alkyl;

each of R⁸ and R⁹ at each occurrence is independently selected from hydrogen, C₁-C₆ alkyl, C₂-C₆ alkenyl, C₃-C₇ cycloalkyl, (C═NR¹³)NR¹¹R¹², S(O₂)C₁-C₆ alkyl, S(O₂)NR¹¹R¹², COR¹³, CO₂R¹³ and CONR¹¹R¹²; wherein the C₁-C₆ alkyl is optionally substituted with one or more hydroxy, halo, C₁-C₆ alkoxy, C₆-C₁₀ aryl, 5- to 10-membered heteroaryl, C₃-C₇ cycloalkyl, 3- to 7-membered heterocycloalkyl, or NR¹¹R¹²;

or R⁸ and R⁹ taken together with the nitrogen they are attached to form a 3- to 10-membered monocyclic or bicyclic ring optionally containing one or more heteroatoms in addition to the nitrogen they are attached to, wherein the ring is optionally substituted with one or more substituents independently selected from halo, C₁-C₆ alkyl, C₁-C₆ haloalkyl, C₁-C₆ alkoxy, oxo, N(C₁-C₆alkyl)₂, NH₂, NH(C₁-C₆ alkyl), and hydroxy;

R¹³ is C₁-C₆ alkyl, C₁-C₆ haloalkyl, or —(Z¹-Z²)ₐ₁-Z³;

each of R¹¹ and R¹² at each occurrence is independently selected from hydrogen, C₁-C₆ alkyl, and —(Z¹-Z²)ₐ₁-Z³;

a1 is 0-10 (e.g., 0-4);

each Z¹ is independently C₁-C₆ alkylene optionally substituted with one or more substituents independently selected from oxo, halo, and hydroxy;

each Z² is independently a bond, NH, N(C₁-C₆ alkyl), —O—, —S—, or 5-10 membered heteroarylene;

Z³ is independently C₆-C₁₀ aryl, C₂-C₆ alkenyl, C₂-C₆ alkynyl, C₃-C₁₀ cycloalkyl, 5- to 10-membered heteroaryl, or 3- to 10-membered heterocycloalkyl, each of which is optionally substituted with one or more substituents independently selected from halo, C₁-C₆ alkyl, C₁₋₆ haloalkyl, C₁-C₆ alkoxy, oxo, N(C₁-C₆alkyl)₂, NH₂, NH(C₁-C₆ alkyl), and hydroxy;

R³ is selected from cyano, hydroxy, C₁-C₆ alkoxy, C(O)OR′, C(O)OR¹⁴, C(O)R′, C(O)NR′R″, C(O)NHR¹⁴, S(O)₀₋₂R′, S(O)₀₋₂R¹⁴, S(O)₁₋₂NR′R″, S(O)₁₋₂NHR¹⁴, and R¹⁴—(C₀-C₂ alkylene)-, wherein the C₀-C₂ alkylene group is optionally substituted with one or more oxo;

R¹⁴ is C₃-C₈ cycloalkyl, 3- to 8-membered heterocycloalkyl, C₂-C₁₀ alkenyl, C₂-C₁₀ alkynyl, 5- to 10-membered monocyclic or bicyclic heteroaryl or C₆-C₁₀ monocyclic or bicyclic aryl, wherein each cycloalkyl, heterocycloalkyl, C₂-C₁₀ alkenyl, C₂-C₁₀ alkynyl, aryl or heteroaryl is optionally independently substituted with 1 or 2 R⁶;

each of R′ and R″ is independently hydrogen or C₁-C₆ alkyl;

R¹⁵ is —(Z⁴-Z⁵)ₐ₂-Z⁶;

a2 is an integer selected from 1-10 (e.g., 1-5);

each Z⁴ is independently selected from —O—, —S—, —NH—, and —N(C₁-C₃ alkyl)-;

provided that the Z⁴ group directly attached to R¹ or R² is —O— or —S—;

each Z⁵ is independently C₁-C₆ alkylene optionally substituted with one or more substituents independently selected from oxo, halo, and hydroxy; and

Z⁶ is OH, OC₁-C₆ alkyl, NH₂, NH(C₁-C₆ alkyl), N(C₁-C₆ alkyl)₂, NHC(O)(C₁-C₆ alkyl), NHC(O)(C₁-C₆ alkoxy), or an optionally substituted group selected from the group consisting of:

C₆-C₁₀ aryl, C₂-C₆ alkenyl, C₂-C₆ alkynyl, C₃-C₁₀ cycloalkyl, 5- to 10-membered heteroaryl, or 3- to 10-membered heterocycloalkyl, each of which is optionally substituted with one or more substituents independently selected from halo, C₁-C₆ alkyl, C₁₋₆ haloalkyl, C₁-C₆ alkoxy, oxo, N(C₁-C₆alkyl)₂, NH₂, NH(C₁-C₆ alkyl), and hydroxy;

or a pharmaceutically acceptable salt thereof.

### Stereochemistry of Formula AA Compounds

The non-limiting exemplary compounds provided herein may encompass various stereochemical forms. The compounds also encompass diastereomers as well as optical isomers, e.g., mixtures of enantiomers including racemic mixtures, as well as individual enantiomers and diastereomers, which arise as a consequence of structural asymmetry in certain compounds. Separation of the individual isomers or selective synthesis of the individual isomers is accomplished by application of various methods which are well known to practitioners in the art. Unless otherwise indicated, when a disclosed compound is named or depicted by a structure without specifying the stereochemistry and has one or more chiral centers, it is understood to represent all possible stereoisomers of the compound.

In one aspect, provided herein is a compound of Formula AA

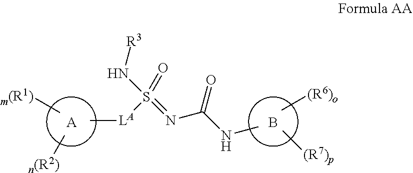

or a pharmaceutically acceptable salt thereof, wherein
L^{A} is a bond;
m=1, or 2;
n=0
A ring is a 5-membered heteroaryl comprising at least 1 heteroatom or heteroatomic group selected from N, NH, and NR¹, S and O;
B ring is

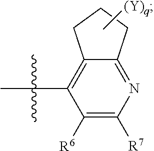

R⁶ is selected from C₁-C₆ alkyl (e.g., methyl, ethyl, or isopropyl) and C₃-C₁₀ cycloalkyl (e.g., cyclopropyl); R⁷ is selected from C₁-C₆ alkyl (e.g., methyl, ethyl, or isopropyl), C₁-C₆ haloalkyl (e.g., trifluoromethyl) and C₃-C₁₀ cycloalkyl (e.g., cyclopropyl or cyclobutyl); or R⁶ and R⁷, taken together with the atoms connecting them, independently form a C₅ carbocyclic ring optionally substituted with one or more C₁-C₆ alkyl (e.g., methyl);
q is 0, 1, or 2; each Y is independently selected from C₁-C₆ alkyl (e.g., methyl); or when two Y are attached to the same carbon, the two Y are taken together with the carbon they are attached to to form a cyclopropyl ring;

R¹ is selected from C₁-C₆ alkyl, C₁-C₆ haloalkyl, C₁-C₆ alkoxy, C₁-C₆ haloalkoxy, halo, and 3- to 7-membered heterocycloalkyl,

wherein the C₁-C₆ alkyl, C₁-C₆ alkoxy, C₁-C₆ haloalkyl, C₆-C₁₀ aryl, 5- to 10-membered heteroaryl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, R¹⁵, C₁-C₆ haloalkoxy, NR⁸R⁹, ═NR¹⁰, COOC₁-C₆ alkyl, CONR⁸R⁹, 3- to 7-membered heterocycloalkyl, C₃-C₇ cycloalkyl, C₆-C₁₀ aryl, 5- to 10-membered heteroaryl, OCOC₁-C₆ alkyl, OCOC₆-C₁₀ aryl, OCO(5- to 10-membered heteroaryl), and OCO(3- to 7-membered heterocycloalkyl);

wherein each C₁-C₆ alkyl substituent and each C₁-C₆ alkoxy substituent of the R¹ or R² C₃-C₇ cycloalkyl or of the R¹ or R² 3- to 7-membered heterocycloalkyl is further optionally independently substituted with one to three hydroxy, —O(C₀-C₃ alkylene)C₆-C₁₀ aryl, halo, NR⁸R⁹, or oxo.

In some embodiments, the compound of Formula AA is an unequal mixture (e.g., a non-racemic mixture when the sulfur is the only stereocenter in Formula AA) of:

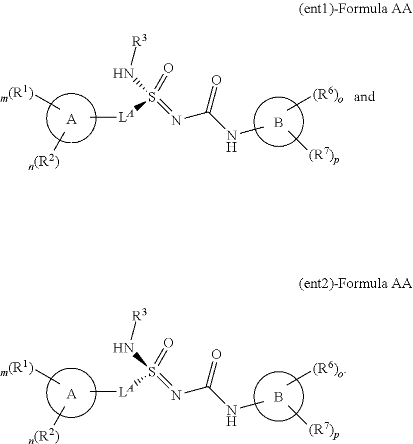

In certain of these embodiments, the mixture is enriched in (ent1)-Formula AA.

In certain of other embodiments, the mixture is enriched in (ent2)-Formula AA.

In certain embodiments, the compound is a compound of (ent1)-Formula AA that is substantially free (e.g., contains less than about 10% of, less than about 5% of, less than about 2% of, less than about 1%, less than about 0.5% of) of (ent2)-Formula AA.

In certain embodiments, the compound is a compound of (ent2)-Formula AA that is substantially free (e.g., contains less than about 10% of, less than about 5% of, less than about 2% of, less than about 1%, less than about 0.5% of) of (ent1)-Formula AA.

In certain embodiments, a Formula AA compound is (+) (dextrorotatory) when in the presence of plane polarized light.

In certain embodiments, a Formula AA compound is (−) (levororotatory) when in the presence of plane polarized light.

In embodiments, the (+) (dextrorotatory) compound is substantially free of (e.g., contains less than about 10% of, less than about 5% of, less than about 2% of, less than about 1%, less than about 0.5%) a corresponding compound (or salt thereof as described herein) that is (−) (levororotatory). In certain embodiments, the (+) (dextrorotatory) compound can be additionally in substantially pure form (e.g., contains less than about 5% of, less than about 2% of, less than about 1%, less than about 0.5% of other substances, including, for example, one or more of other Formula AA compounds, non-Formula AA compounds, or biological media).

In embodiments, the (−) (levororotatory) compound is substantially free of (e.g., contains less than about 10% of, less than about 5% of, less than about 2% of, less than about 1%, less than about 0.5%) a corresponding compound (or salt thereof as described herein) that is (+) (dextrorotatory). In certain embodiments, the (−) (levororotatory) compound can be additionally in substantially pure form (e.g., contains less than about 5% of, less than about 2% of, less than about 1%, less than about 0.5% of other substances, including, for example, one or more of other Formula AA compounds, non-Formula AA compounds, or biological media).

In some embodiments, the compound of Formula AA-CN is an unequal mixture (e.g., a non-racemic mixture when the sulfur is the only stereocenter in Formula AA-CN) of:

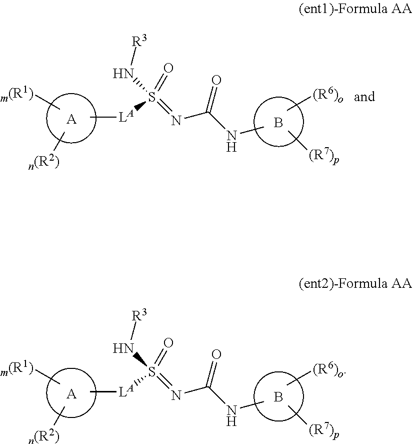

In certain of these embodiments, the mixture is enriched in (ent1)-Formula AA-CN.

In certain of other embodiments, the mixture is enriched in (ent2)-Formula AA-CN.

In certain embodiments, the compound is a compound of (ent1)-Formula AA-CN that is substantially free (e.g., contains less than about 10% of, less than about 5% of, less than about 2% of, less than about 1%, less than about 0.5% of) of (ent2)-Formula AA-CN.

In certain embodiments, the compound is a compound of (ent2)-Formula AA-CN that is substantially free (e.g., contains less than about 10% of, less than about 5% of, less than about 2% of, less than about 1%, less than about 0.5% of) of (ent1)-Formula AA-CN.

The inventors have discovered that in the corresponding N—H (R³═H) compounds, activity primarily resides in only one of the two sulfur enantiomers or epimers; however, surprisingly, when said hydrogen atom is replaced with a cyano group, activity resides primarily in the opposing enantiomer or epimer.
In some embodiments the variables shown in the formulae herein are as follows: The moiety

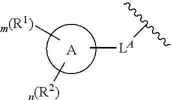

In some embodiments,

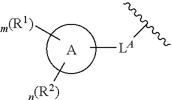

is as defined for (AA-1):

L^{A} is a bond;

m=0, 1, or 2;

n=0, 1, or 2, wherein m+n≥2; and

A is a 5- to 10-membered monocyclic or bicyclic heteroaryl wherein one ring atom is a heteroatom selected from O, N, and S.

In some embodiments,

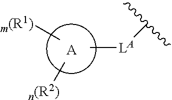

is as defined for (AA-2):

L^{A} is a bond;

m=0, 1, or 2;

n=0, 1, or 2; and

A is a 5- to 10-membered monocyclic or bicyclic heteroaryl wherein from 2-3 ring atoms are heteroatoms, each independently selected from O, N, and S; or a C₇-C₁₀ bicyclic aryl.

In some embodiments,

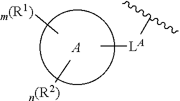

is as defined for (AA-3):

L^{A} is a C₁-C₈ alkylene optionally substituted with halo;

m=0, 1, or 2;

n=0, 1, or 2; and

A is a 5- to 10-membered monocyclic or bicyclic heteroaryl wherein one ring atom is a heteroatom selected from O, N, and S; or a C₇-C₁₀ bicyclic aryl.

In some embodiments,

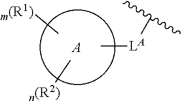

is as defined for (AA-4):

L^{A} is a C₁-C₈ alkylene optionally substituted with halo;

m=0, 1, or 2; and

n=0, 1, or 2; wherein m+n≥2; and

A is phenyl.

The Variables m and n

In some embodiments m=0, 1, or 2.

In some embodiments m=0 or 1.

In some embodiments m=1 or 2.

In some embodiments m=0 or 2.

In some embodiments m=0.

In some embodiments m=1.

In some embodiments m=2.

In some embodiments n=0, 1, or 2.

In some embodiments n=0 or 1.

In some embodiments n=1 or 2.

In some embodiments n=0 or 2.

In some embodiments n=0.

In some embodiments n=1.

In some embodiments n=2.

In some embodiments, m=0, and n=0.

In some embodiments, m=1, and n=0.

In some embodiments, m=1, and n=1.

The Ring A and Substitutions on the Ring A

In some embodiments, A is a 5- to 10-membered (e.g., 5- to 6-membered) monocyclic or bicyclic heteroaryl or a C₆-C₁₀ (e.g., C₆) monocyclic or bicyclic aryl, such as phenyl.

In some embodiments, A is a 5- to 10-membered (e.g., 5- to 6-membered) monocyclic or bicyclic heteroaryl.

In some embodiments, A is a 5-membered heteroaryl.

In some embodiments, A is a 5-membered heteroaryl containing a sulfur and optionally one or more nitrogens.

In some embodiments, A is a 6-membered heteroaryl.

In some embodiments, A is a C₆-C₁₀ monocyclic or bicyclic aryl.

In some embodiments, A is phenyl optionally substituted with 1 or 2 R¹ and optionally substituted with 1 or 2 R².

In some embodiments, A is naphthyl optionally substituted with 1 or 2 R¹ and optionally substituted with 1 or 2 R².

In some embodiments, A is furanyl optionally substituted with 1 or 2 R¹ and optionally substituted with 1 R².

In some embodiments, A is furanyl optionally substituted with 1 R¹ and optionally substituted with 1 or 2 R².

In some embodiments, A is thiophenyl optionally substituted with 1 or 2 R¹ and optionally substituted with 1 or 2 R².

In some embodiments, A is oxazolyl optionally substituted with 1 or 2 R¹ and optionally substituted with 1 or 2 R².

In some embodiments, A is thiazolyl optionally substituted with 1 or 2 R¹ and optionally substituted with 1 or 2 R².

In some embodiments, A is oxazolyl optionally substituted with 2 R¹ or optionally substituted with 2 R².

In some embodiments, A is thiazolyl optionally substituted with 2 R¹ or optionally substituted with 2 R².

In some embodiments, A is pyrazolyl optionally substituted with 1 or 2 R¹ and optionally substituted with 1 or 2 R².

In some embodiments, A is pyrazolyl optionally substituted with 1 R¹ and optionally substituted with 1 or 2 R².

In some embodiments, A is pyrazolyl optionally substituted with 1 or 2 R¹ and optionally substituted with 1 R².

In some embodiments, A is imidazolyl optionally substituted with 1 or 2 R¹ and optionally substituted with 1 or 2 R².

In some embodiments, A is pyrrolyl optionally substituted with 1 or 2 R¹ and optionally substituted with 1 or 2 R².

In some embodiments, A is oxazolyl optionally substituted with 1 or 2 R¹ and optionally substituted with 1 or 2 R².

In some embodiments, A is furanyl optionally substituted with 1 or 2 R¹ and optionally substituted with 1 or 2 R².

In some embodiments, A is isoxazolyl optionally substituted with 1 or 2 R¹ and optionally substituted with 1 or 2 R².

In some embodiments, A is isothiazolyl optionally substituted with 1 or 2 R¹ and optionally substituted with 1 or 2 R².

In some embodiments, A is triazolyl (e.g., 1,2,3-triazolyl or 1,2,4-triazolyl) optionally substituted with 1 R¹ and optionally substituted with 1 R².

In some embodiments, A is pyridyl optionally substituted with 1 or 2 R¹ and optionally substituted with 1 or 2 R².

In some embodiments, A is pyridinyl optionally substituted with 1 or 2 R¹ and optionally substituted with 1 or 2 R².

In some embodiments, A is pyridimidinyl optionally substituted with 1 or 2 R¹ and optionally substituted with 1 or 2 R².

In some embodiments, A is pyrazinyl optionally substituted with 1 or 2 R¹ and optionally substituted with 1 or 2 R².

In some embodiments, A is pyridazinyl optionally substituted with 1 or 2 R¹ and optionally substituted with 1 or 2 R².

In some embodiments, A is triazinyl optionally substituted with 1 or 2 R¹ and optionally substituted with 1 or 2 R².

In some embodiments, A is indazolyl optionally substituted with 1 or 2 R¹ and optionally substituted with 1 or 2 R².

In some embodiments, A is phenyl substituted with 1 R¹ and optionally substituted with 1 R².

In some embodiments, A is naphthyl substituted with 1 R¹ and optionally substituted with 1 R².

In some embodiments, A is furanyl substituted with 1 R¹ and optionally substituted with 1 R².

In some embodiments, A is thiophenyl substituted with 1 R¹ and optionally substituted with 1 R².

In some embodiments, A is oxazolyl substituted with 1 R¹ and optionally substituted with 1 R².

In some embodiments, A is thiazolyl substituted with 1 R¹ and optionally substituted with 1 R².

In some embodiments, A is pyrazolyl substituted with 1 R¹ and optionally substituted with 1 R².

In some embodiments, A is imidazolyl substituted with 1 R¹ and substituted with 1 R².

In some embodiments, A is pyrrolyl substituted with 1 R¹ and substituted with 1 R².

In some embodiments, A is oxazolyl substituted with 1 R¹ and substituted with 1 R².

In some embodiments, A is furanyl substituted with 1 R¹ and substituted with 1 R².

In some embodiments, A is isoxazolyl substituted with 1 R¹ and substituted with 1 R².

In some embodiments, A is isothiazolyl substituted with 1 R¹ and substituted with 1 R².

In some embodiments, A is triazolyl (e.g., 1,2,3-triazolyl or 1,2,4-triazolyl) substituted with 1 R¹ and substituted with 1 R².

In some embodiments, A is pyridyl substituted with 1 R¹ and optionally substituted with 1 R².

In some embodiments, A is pyridimidinyl substituted with 1 R¹ and substituted with 1 R².

In some embodiments, A is pyrazinyl substituted with 1 R¹ and substituted with 1 R².

In some embodiments, A is pyridazinyl substituted with 1 R¹ and substituted with 1 R².

In some embodiments, A is triazinyl substituted with 1 R¹ and substituted with 1 R².

In some embodiments, A is indazolyl optionally substituted with 1 R¹ and optionally substituted with 1 R².

In some embodiments, A is phenyl substituted with 1 R¹ and substituted with 1 R².

In some embodiments, A is furanyl substituted with 1 R¹ and substituted with 1 R².

In some embodiments, A is thiophenyl substituted with 1 R¹ and substituted with 1 R².

In some embodiments, A is oxazolyl substituted with 1 R¹ and substituted with 1 R².

In some embodiments, A is thiazolyl substituted with 1 R¹ and substituted with 1 R².

In some embodiments, A is pyrazolyl substituted with 1 R¹ and substituted with 1 R².

In some embodiments, A is pyridyl substituted with 1 R¹ and substituted with 1 R².

In some embodiments, A is pyridazinyl substituted with 1 R¹ and substituted with 1 R².

In some embodiments, A is phenyl, m is 0 or 1, and n is 0, 1, or 2.

In some embodiments, A is furanyl, m is 0 or 1, and n is 0, 1, or 2.

In some embodiments, A is thiophenyl, m is 0 or 1, and n is 0, 1, or 2.

In some embodiments, A is oxazolyl, m is 0 or 1, and n is 0, 1, or 2.

In some embodiments, A is thiazolyl, m is 0 or 1, and n is 0, 1, or 2.

In some embodiments, A is pyrazolyl, m is 0 or 1, and n is 0, 1, or 2.

In some embodiments, A is pyridyl, m is 0 or 1, and n is 0, 1, or 2.

In some embodiments, A is pyridazinyl, m is 0 or 1, and n is 0, 1, or 2.

In some embodiments, A is indazolyl, m is 0 or 1, and n is 0, 1, or 2.

In some embodiments, A is phenyl, m is 0, and n is 0 or 1.

In some embodiments, A is furanyl, m is 0, and n is 0 or 1.

In some embodiments, A is thiophenyl, m is 0, and n is 0 or 1.

In some embodiments, A is oxazolyl, m is 0, and n is 0 or 1.

In some embodiments, A is thiazolyl, m is 0, and n is 0 or 1.

In some embodiments, A is pyrazolyl, m is 0, and n is 0 or 1.

In some embodiments, A is pyridyl, m is 0, and n is 0 or 1.

In some embodiments, A is thiazolyl, m is 1, and n is 1.

In some embodiments, A is pyrazolyl, m is 1 or 2, and n is 1 or 2.

In some embodiments, A is imidazolyl, m is 1 or 2, and n is 1 or 2.

In some embodiments, A is pyrrolyl, m is 1 or 2, and n is 1 or 2.

In some embodiments, A is oxazolyl, m is 1, and n is 1.

In some embodiments, A is furanyl, m is 1 or 2, and n is 1 or 2.

In some embodiments, A is isoxazolyl, m is 1, and n is 1.

In some embodiments, A is isothiazolyl, m is 1, and n is 1.

In some embodiments, A is triazolyl (e.g., 1,2,3-triazolyl or 1,2,4-triazolyl), m is 1, and n is 1.

In some embodiments, A is pyridinyl, m is 1 or 2, and n is 1 or 2.

In some embodiments, A is pyridimidinyl, m is 1 or 2, and n is 1 or 2.

In some embodiments, A is pyrazinyl, m is 1 or 2, and n is 1 or 2.

In some embodiments, A is pyridazinyl, m is 1 or 2, and n is 1 or 2.

In some embodiments, A is triazinyl, m is 1, and n is 1.

In some embodiments, A is one of the rings disclosed hereinbelow optionally substituted as disclosed hereinbelow, wherein in each case the bond that is shown as being broken by the wavy line [symbol] connects A to the S(O)(NR³R³)═N moiety of Formula AA.

In some embodiments, the optionally substituted ring A

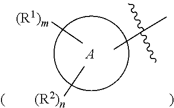

is

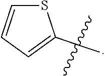

In some embodiments, the optionally substituted ring A is

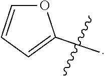

In some embodiments, the optionally substituted ring A is

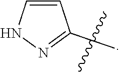

In some embodiments, the optionally substituted ring A is

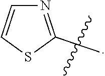

In some embodiments, the optionally substituted ring A is

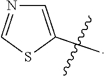

In some embodiments, the optionally substituted ring A is

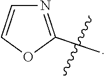

In some embodiments, the optionally substituted ring A is

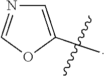

In some embodiments, the optionally substituted ring A is

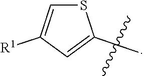

In some embodiments, the optionally substituted ring A is

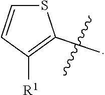

In some embodiments, the optionally substituted ring A is

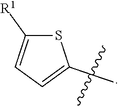

In some embodiments, the optionally substituted ring A is

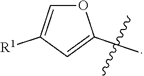

In some embodiments, the optionally substituted ring A is

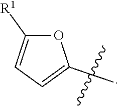

In some embodiments, the optionally substituted ring A is

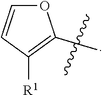

In some embodiments, the optionally substituted ring A is

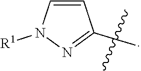

In some embodiments, the optionally substituted ring A is

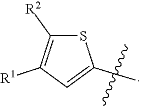

In some embodiments, the optionally substituted ring A is

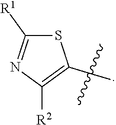

In some embodiments, the optionally substituted ring A is

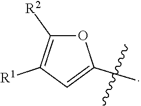

In some embodiments, the optionally substituted ring A is

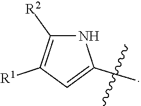

In some embodiments, the optionally substituted ring A is

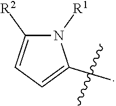

In some embodiments, the optionally substituted ring A is

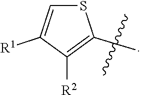

In some embodiments, the optionally substituted ring A is

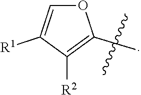

In some embodiments, the optionally substituted ring A is

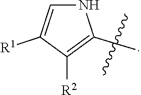

In some embodiments, the optionally substituted ring A is

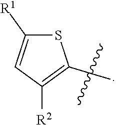

In some embodiments, the optionally substituted ring A is

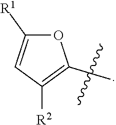

In some embodiments, the optionally substituted ring A is

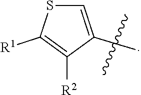

In some embodiments, the optionally substituted ring A is

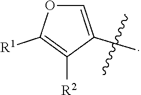

In some embodiments, the optionally substituted ring A is

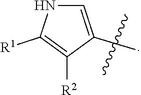

In some embodiments, the optionally substituted ring A is

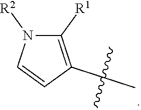

In some embodiments, the optionally substituted ring A is

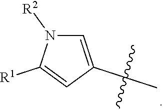

In some embodiments, the optionally substituted ring A is

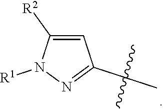

In some embodiments, the optionally substituted ring A is

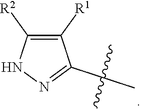

In some embodiments, the optionally substituted ring A is

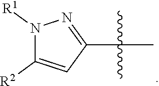

In some embodiments, the optionally substituted ring A is

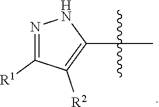

In some embodiments, the optionally substituted ring A is

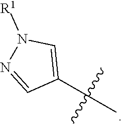

In some embodiments, the optionally substituted ring A is

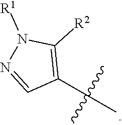

In some embodiments, the optionally substituted ring A is

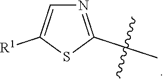

In some embodiments, the optionally substituted ring A is

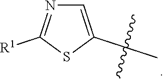

In some embodiments, the optionally substituted ring A is

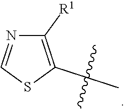

In some embodiments, the optionally substituted ring A is

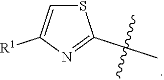

In some embodiments, the optionally substituted ring A is

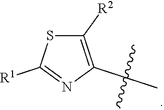

In some embodiments, the optionally substituted ring A is

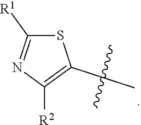

In some embodiments, the optionally substituted ring A is

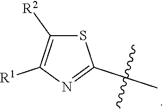

In some embodiments, the optionally substituted ring A is

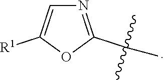

In some embodiments, the optionally substituted ring A is

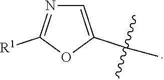

In some embodiments, the optionally substituted ring A is

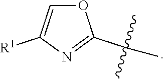

In some embodiments, the optionally substituted ring A is

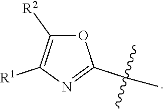

In some embodiments, the optionally substituted ring A is

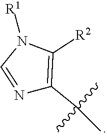

In some embodiments, the optionally substituted ring A is

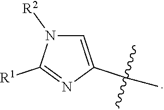

In some embodiments, the optionally substituted ring A is

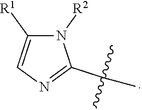

In some embodiments, the optionally substituted ring A is

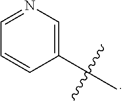 [SMILES: *c1cccnc1]

In some embodiments, the optionally substituted ring A is

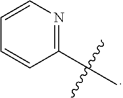 [SMILES: *c1ccccn1]

In some embodiments, the optionally substituted ring A is

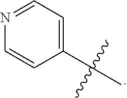 [SMILES: *c1ccncc1]

In some embodiments, the optionally substituted ring A is

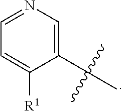

In some embodiments, the optionally substituted ring A is

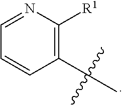

In some embodiments, the optionally substituted ring A is

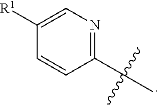

In some embodiments, the optionally substituted ring A is

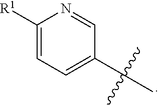

In some embodiments, the optionally substituted ring A is

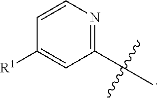

In some embodiments, the optionally substituted ring A is

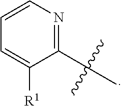

In some embodiments, the optionally substituted ring A is

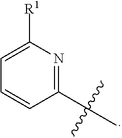

In some embodiments, the optionally substituted ring A is

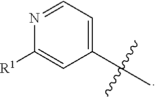

In some embodiments, the optionally substituted ring A is

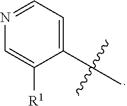

In some embodiments, the optionally substituted ring A is

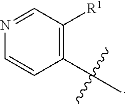

In some embodiments, the optionally substituted ring A is

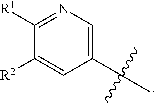

In some embodiments, the optionally substituted ring A is

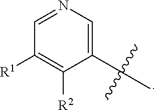

In some embodiments, the optionally substituted ring A is

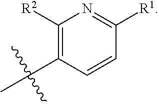

In some embodiments, the optionally substituted ring A is

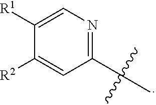

In some embodiments, the optionally substituted ring A is

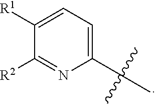

In some embodiments, the optionally substituted ring A is

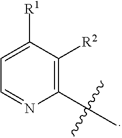

In some embodiments, the optionally substituted ring A is

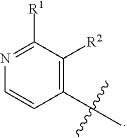

In some embodiments, the optionally substituted ring A is

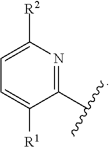

In some embodiments, the optionally substituted ring A is

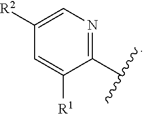

In some embodiments, the optionally substituted ring A is

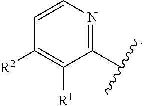

In some embodiments, the optionally substituted ring A is

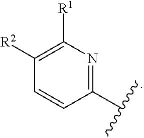

In some embodiments, the optionally substituted ring A is

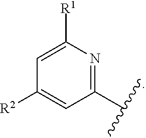

In some embodiments, the optionally substituted ring A is

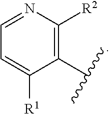

In some embodiments, the optionally substituted ring A is

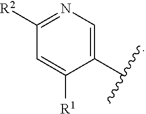

In some embodiments, the optionally substituted ring A is

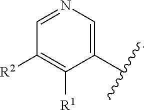

In some embodiments, the optionally substituted ring A is

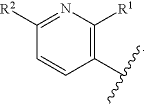

In some embodiments, the optionally substituted ring A is

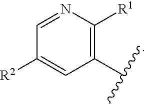

In some embodiments, the optionally substituted ring A is

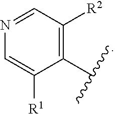

In some embodiments, the optionally substituted ring A is

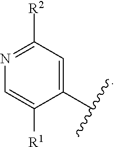

In some embodiments, the optionally substituted ring A is

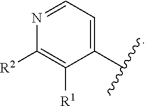

In some embodiments, the optionally substituted ring A is

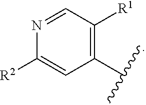

In some embodiments, the optionally substituted ring A is

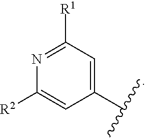

In some embodiments, the optionally substituted ring A is

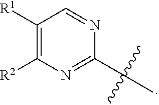

In some embodiments, the optionally substituted ring A is

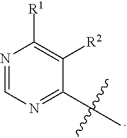

In some embodiments, the optionally substituted ring A is

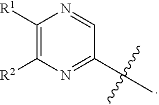

In some embodiments, the optionally substituted ring A is

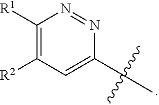

In some embodiments, the optionally substituted ring A is

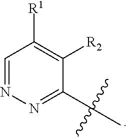

In some embodiments, the optionally substituted ring A is

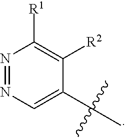

In some embodiments, the optionally substituted ring A is

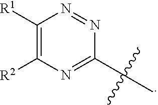

In some embodiments, the optionally substituted ring A is

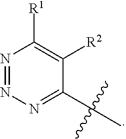

In some embodiments, the optionally substituted ring A is

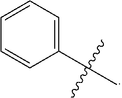 [SMILES: *c1ccccc1]

In some embodiments, the optionally substituted ring A is

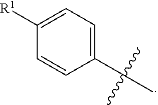

In some embodiments, the optionally substituted ring A is

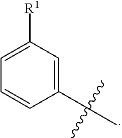

In some embodiments, the optionally substituted ring A is

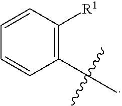

In some embodiments, the optionally substituted ring A is

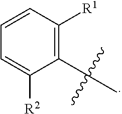

In some embodiments, the optionally substituted ring A is

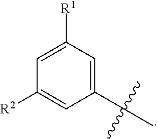

In some embodiments, the optionally substituted ring A is

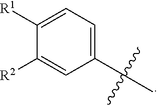

In some embodiments, the optionally substituted ring A is

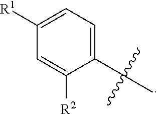

In some embodiments, the optionally substituted ring A is

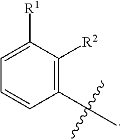

In some embodiments, the optionally substituted ring A is

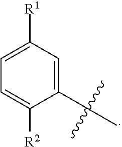

In some embodiments, the optionally substituted ring A is

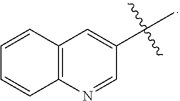 [SMILES: *c1cnc2ccccc2c1]

In some embodiments, the optionally substituted ring A is

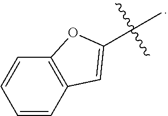 [SMILES: *c1cc2ccccc2o1]

In some embodiments, the optionally substituted ring A is

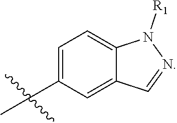

In some embodiments, the optionally substituted ring A is

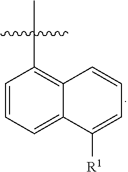

In some embodiments, the optionally substituted ring A is

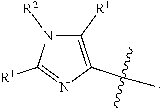

In some embodiments, the optionally substituted ring A is

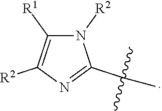

In some embodiments, the optionally substituted ring A is

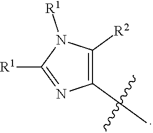

In some embodiments, the optionally substituted ring A is

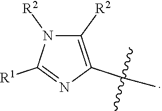

In some embodiments, the optionally substituted ring A is

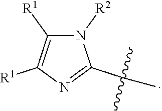

In some embodiments, the optionally substituted ring A is

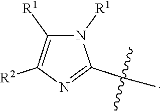

In some embodiments, the optionally substituted ring A is

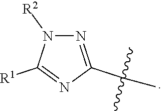

In some embodiments, the optionally substituted ring A is

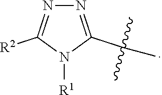

In some embodiments, the optionally substituted ring A is

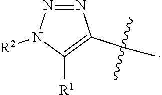

In some embodiments, the optionally substituted ring A is

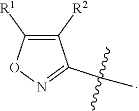

In some embodiments, the optionally substituted ring A is

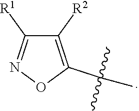

In some embodiments, the optionally substituted ring A is

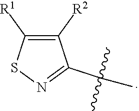

In some embodiments, the optionally substituted ring A is

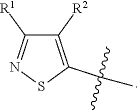

In some embodiments, the optionally substituted ring A is

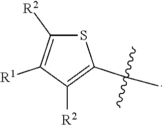

In some embodiments, the optionally substituted ring A is

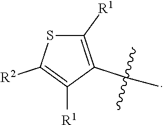

In some embodiments, the optionally substituted ring A is

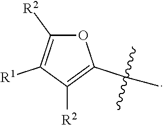

In some embodiments, the optionally substituted ring A is

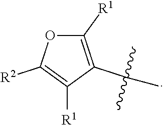

In some embodiments, the optionally substituted ring A is

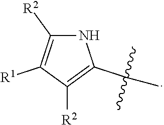

In some embodiments, the optionally substituted ring A is

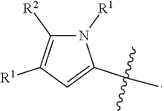

In some embodiments, the optionally substituted ring A is

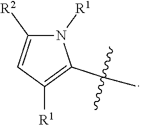

In some embodiments, the optionally substituted ring A is

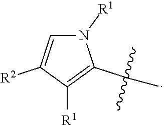

In some embodiments, the optionally substituted ring A is

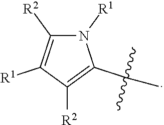

In some embodiments, the optionally substituted ring A is

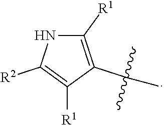

In some embodiments, the optionally substituted ring A is

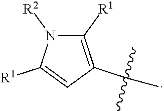

In some embodiments, the optionally substituted ring A is

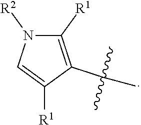

In some embodiments, the optionally substituted ring A is

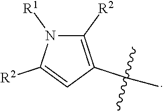

In some embodiments, the optionally substituted ring A is

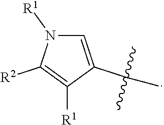

In some embodiments, the optionally substituted ring A is

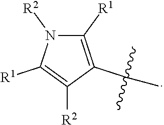

In some embodiments, the optionally substituted ring A is

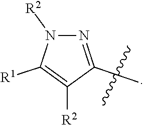

In some embodiments, the optionally substituted ring A is

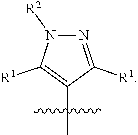

In some embodiments, the optionally substituted ring A is

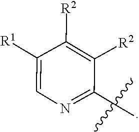

In some embodiments, the optionally substituted ring A is

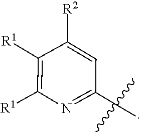

In some embodiments, the optionally substituted ring A is

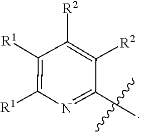

In some embodiments, the optionally substituted ring A is

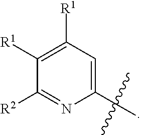

In some embodiments, the optionally substituted ring A is

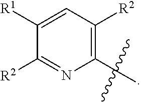

In some embodiments, the optionally substituted ring A is

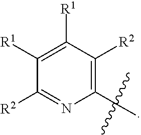

In some embodiments, the optionally substituted ring A is

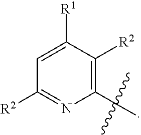

In some embodiments, the optionally substituted ring A is

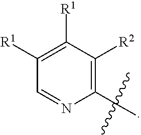

In some embodiments, the optionally substituted ring A is

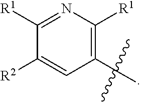

In some embodiments, the optionally substituted ring A is

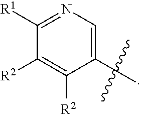

In some embodiments, the optionally substituted ring A is

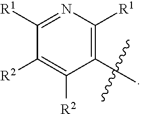

In some embodiments, the optionally substituted ring A is

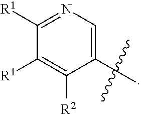

In some embodiments, the optionally substituted ring A is

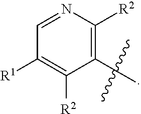

In some embodiments, the optionally substituted ring A is

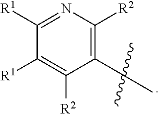

In some embodiments, the optionally substituted ring A is

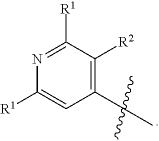

In some embodiments, the optionally substituted ring A is

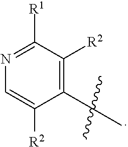

In some embodiments, the optionally substituted ring A is

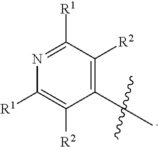

In some embodiments, the optionally substituted ring A is

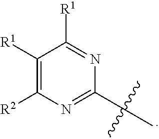

In some embodiments, the optionally substituted ring A is

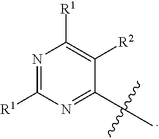

In some embodiments, the optionally substituted ring A is

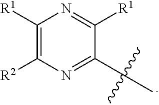

In some embodiments, the optionally substituted ring A is

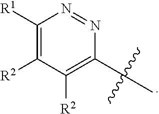

In some embodiments, the optionally substituted ring A is

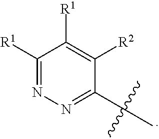

In some embodiments, the optionally substituted ring A is

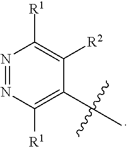

In some embodiments, the optionally substituted ring A is

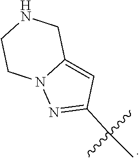 [SMILES: *c1cc2n(n1)CCNC2]

In some embodiments, the optionally substituted ring A is

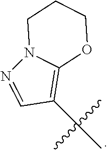 [SMILES: *c1cnn2c1OCCC2]

In some embodiments, the optionally substituted ring A is

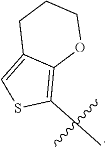 [SMILES: *c1scc2c1OCCC2]

In some embodiments, the optionally substituted ring A is

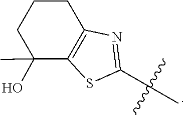 [SMILES: *c1nc2c(s1)C(C)(O)CCC2]

In some embodiments, the optionally substituted ring A is selected from the group consisting of:

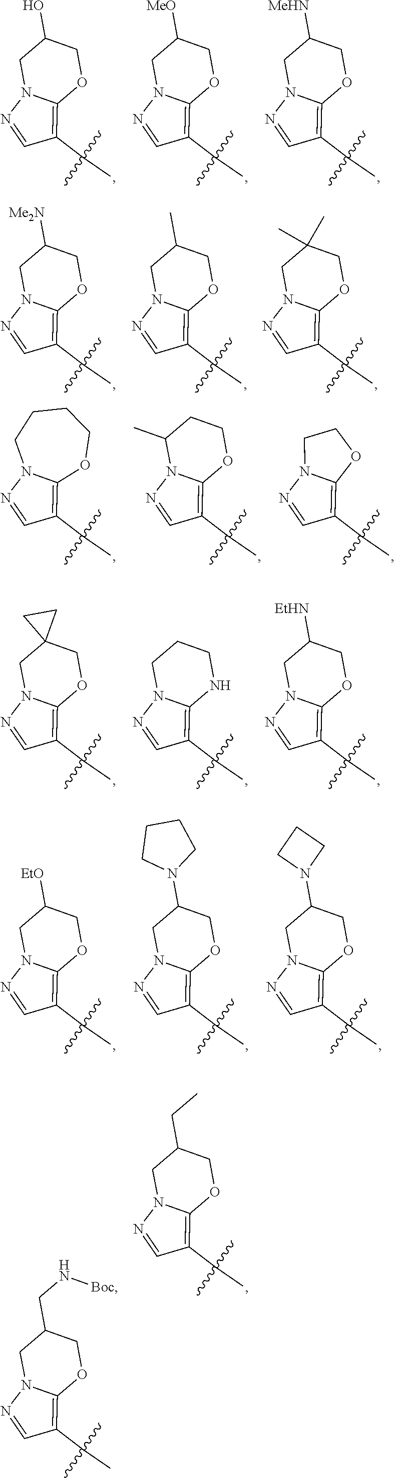

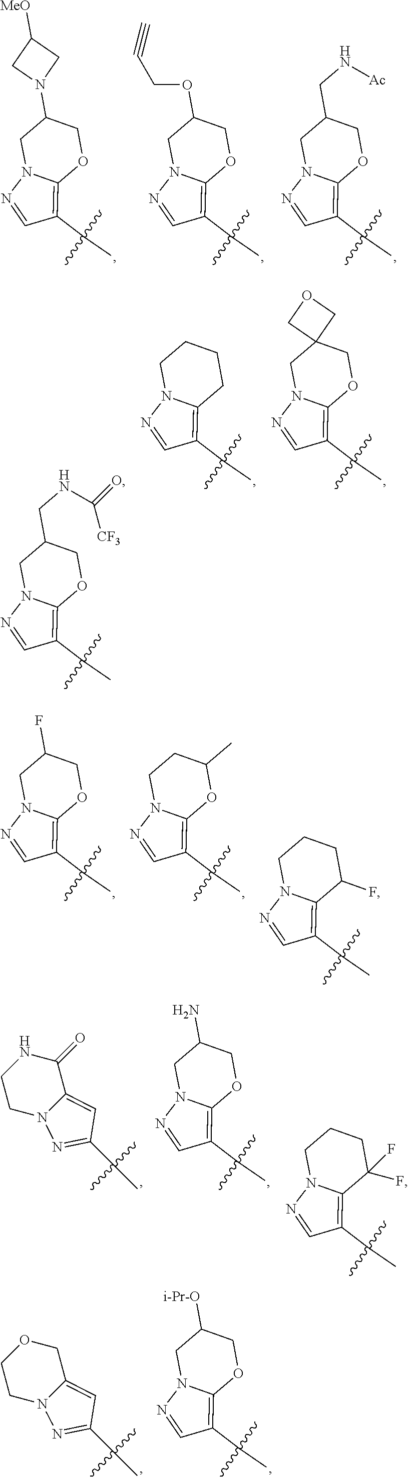

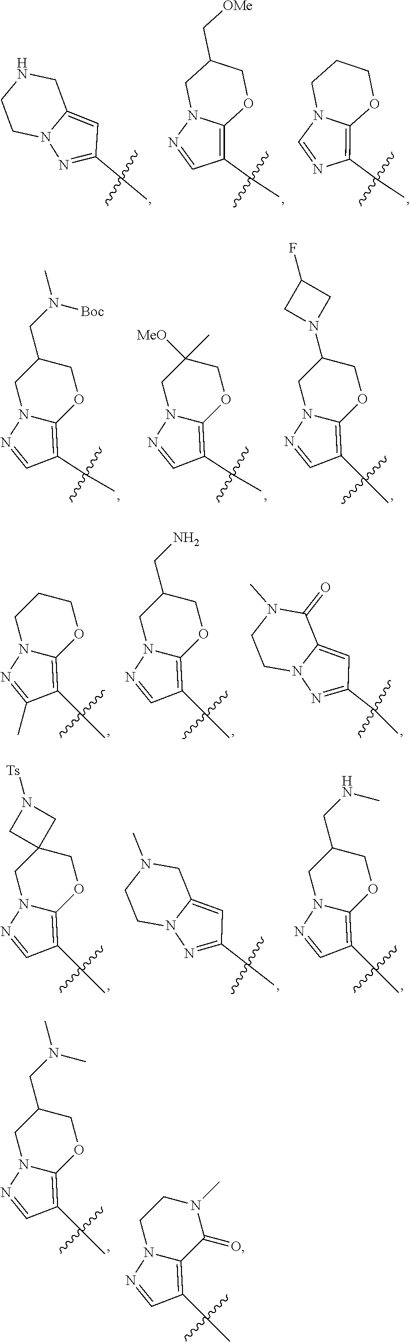

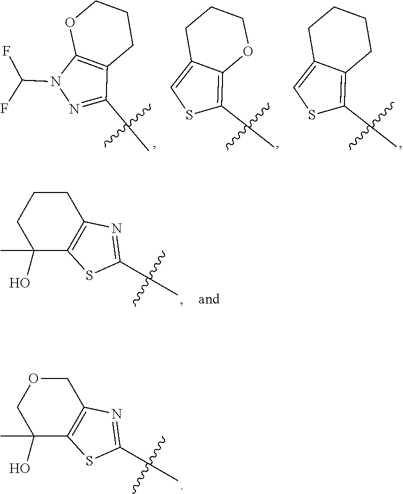 [SMILES: *c1nn(C(F)F)c2c1CCCO2 ; *c1scc2c1OCCC2 ; *c1scc2c1CCCC2 ; *c1nc2c(s1)C(C)(O)CCC2 ; *c1nc2c(s1)C(C)(O)COC2]

In some embodiments, the optionally substituted ring A is selected from the group consisting of:

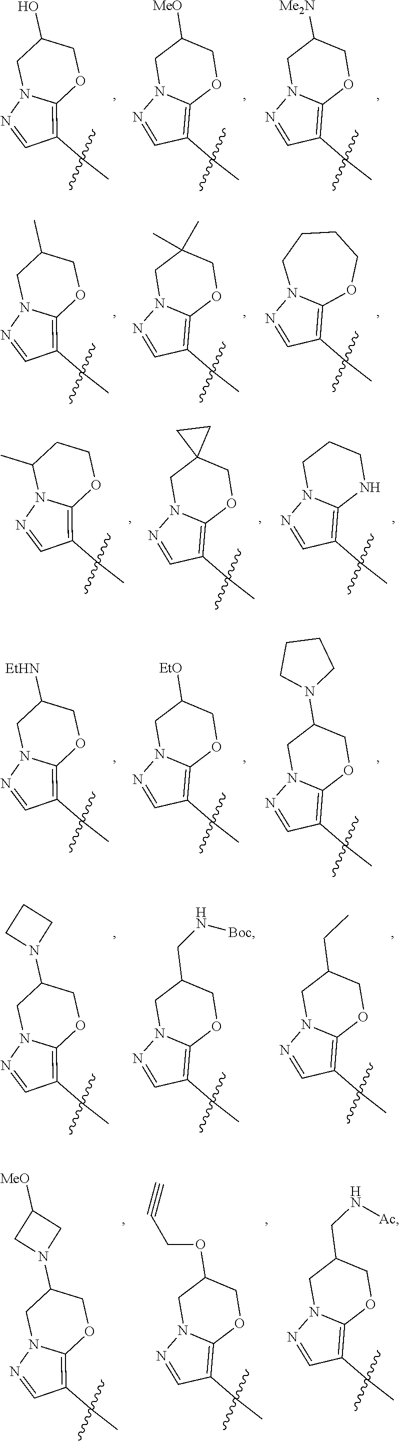

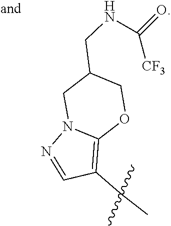 [SMILES: *c1cnn2c1OCC(CNC(=O)C(F)(F)F)C2]

In some embodiments, the optionally substituted ring A is selected from the group consisting of:

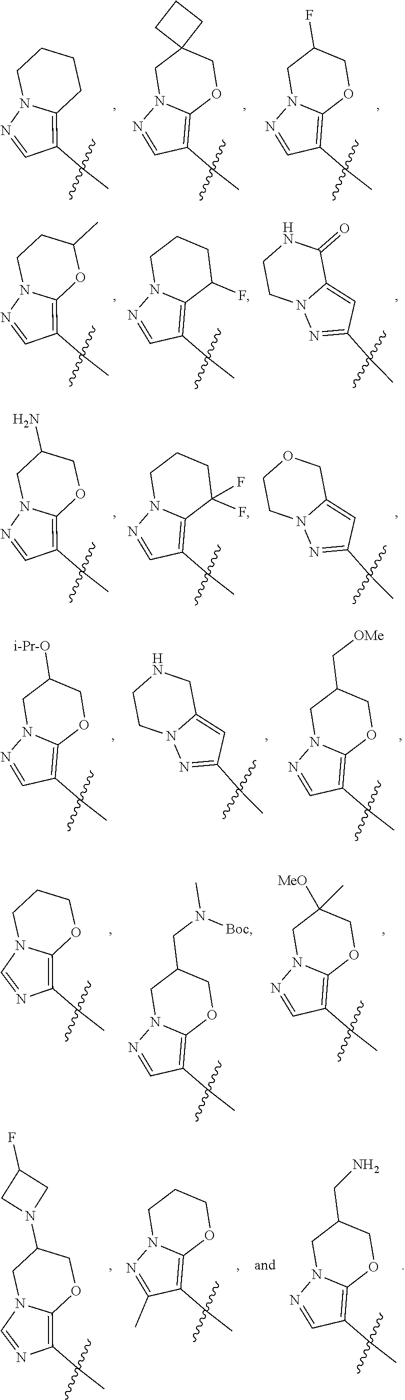

In some embodiments, the optionally substituted ring A is selected from the group consisting of:

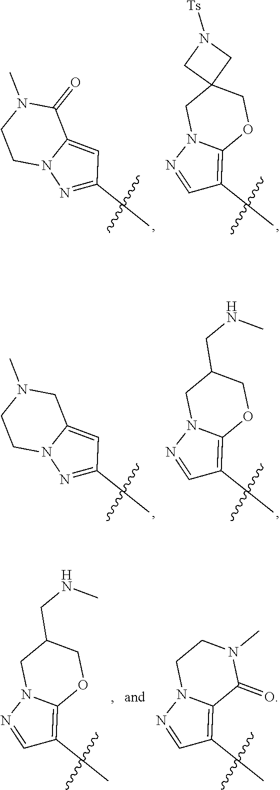 [SMILES: *c1cc2n(n1)CCN(C)C2=O ; *c1cnn2c1OCC1(CN(S(=O)(=O)c3ccc(C)cc3)C1)C2 ; *c1cc2n(n1)CCN(C)C2 ; *c1cnn2c1OCC(CNC)C2 ; *c1cnn2c1OCC(CNC)C2 ; *c1cnn2c1C(=O)N(C)CC2]

In some embodiments, the optionally substituted ring A is selected from the group consisting of:

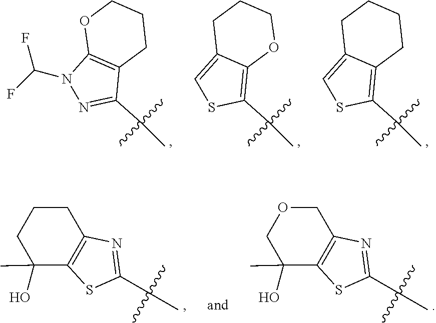 [SMILES: *c1nn(C(F)F)c2c1CCCO2 ; *c1scc2c1OCCC2 ; *c1scc2c1CCCC2 ; *c1nc2c(s1)C(C)(O)CCC2 ; *c1nc2c(s1)C(C)(O)COC2]

In some embodiments, when ring A is phenyl, then R¹ and R² are each independently selected from C₃ alkyl, C₅-C₆ alkyl, C₁-C₆ haloalkyl, C₁-C₆ alkoxy, C₁-C₆ haloalkoxy, F, I, CN, NO₂, COC₂-C₆ alkyl, CO-C₆-C₁₀ aryl, CO(5- to 10-membered heteroaryl), CO₂C₁-C₆ alkyl, CO₂C₃-C₈ cycloalkyl, OCOC₂-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, NHCOC₁-C₆ alkyl, NHCOC₆-C₁₀ aryl, NHCO(5- to 10-membered heteroaryl), NHCO(3- to 7-membered heterocycloalkyl), NHCOC₂-C₆ alkynyl, NHCOOCC₁-C₆ alkyl, NH—(C═NR¹³)NR¹¹R¹², CONR⁸R⁹, SF₅, SC₁-C₆ alkyl, S(O₂)C₁-C₆ alkyl, S(O₂)NR¹¹R¹², S(O)C₁-C₆ alkyl, C₃-C₇ cycloalkyl, and 3- to 7-membered heterocycloalkyl, wherein the C₁-C₆ alkyl, C₁-C₆ haloalkyl, C₃-C₇ cycloalkyl and 3- to 7-membered heterocycloalkyl are optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;
wherein each C₁-C₆ alkyl substituent and each C₁-C₆ alkoxy substituent of the R¹ or R² C₃-C₇ cycloalkyl or of the R¹ or R² 3- to 7-membered heterocycloalkyl is further optionally independently substituted with one to three hydroxy, halo, NR⁸R⁹, or oxo;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;

or one pair of R¹ and R² on adjacent atoms, taken together with the atoms connecting them, independently form one monocyclic or bicyclic C₄ or C₆-C₁₂ carbocyclic ring or one monocyclic or bicyclic 5- to-12-membered heterocyclic ring containing 1-3 heteroatoms independently selected from O, N, and S, wherein the carbocyclic ring or heterocyclic ring is optionally independently substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR⁸R⁹, ═NR¹⁰, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR⁸R⁹, wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR⁸R⁹, ═NR¹⁰, COOC₁-C₆ alkyl, C₆-C₁₀ aryl, and CONR⁸R⁹.

In some embodiments, R¹ and R² are each independently selected from C₃ alkyl, C₅-C₆ alkyl, C₁-C₆ haloalkyl, C₁-C₆ alkoxy, C₁-C₆ haloalkoxy, F, I, CN, NO₂, COC₂-C₆ alkyl, CO-C₆-C₁₀ aryl, CO(5- to 10-membered heteroaryl), CO₂C₁-C₆ alkyl, CO₂C₃-C₈ cycloalkyl, OCOC₂-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, NHCOC₁-C₆ alkyl, NHCOC₆-C₁₀ aryl, NHCO(5- to 10-membered heteroaryl), NHCO(3- to 7-membered heterocycloalkyl), NHCOC₂-C₆ alkynyl, NHCOOCC₁-C₆ alkyl, NH—(C═NR¹³)NR¹¹R¹², CONR⁸R⁹, SF₅, SC₁-C₆ alkyl, S(O₂)C₁-C₆ alkyl, S(O₂)NR¹¹R¹², S(O)C₁-C₆ alkyl, C₃-C₇ cycloalkyl, and 3- to 7-membered heterocycloalkyl, wherein the C₁-C₆ alkyl, C₁-C₆ haloalkyl, C₃-C₇ cycloalkyl and 3- to 7-membered heterocycloalkyl are optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;
wherein each C₁-C₆ alkyl substituent and each C₁-C₆ alkoxy substituent of the R¹ or R² C₃-C₇ cycloalkyl or of the R¹ or R² 3- to 7-membered heterocycloalkyl is further optionally independently substituted with one to three hydroxy, halo, NR⁸R⁹, or oxo;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;

or one pair of R¹ and R² on adjacent atoms, taken together with the atoms connecting them, independently form one monocyclic or bicyclic C₄ or C₆-C₁₂ carbocyclic ring or one monocyclic or bicyclic 5- to-12-membered heterocyclic ring containing 1-3 heteroatoms independently selected from O, N, and S, wherein the carbocyclic ring or heterocyclic ring is optionally independently substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR⁸R⁹, ═NR¹⁰, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR⁸R⁹, wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR⁸R⁹, ═NR¹⁰, COOC₁-C₆ alkyl, C₆-C₁₀ aryl, and CONR⁸R⁹.

In some embodiments of the compound of Formula AA, when ring A is pyridyl, then R¹ and R² are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO-C₆-C₁₀ aryl, CO(5- to 10-membered heteroaryl), CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₂-C₆ alkyl, N(C₁-C₆ alkyl)₂, NHCOC₁-C₆ alkyl, NHCOC₆-C₁₀ aryl, NHCO(5- to 10-membered heteroaryl), NHCO(3- to 7-membered heterocycloalkyl), NHCOC₂-C₆ alkynyl, NHCOOCC₁-C₆ alkyl, NH—(C═NR¹³)NR¹¹R¹², CONR⁸R⁹, SF₅, SC₁-C₆ alkyl, S(O₂)C₁-C₆ alkyl, S(O₂)NR¹¹R¹², S(O)C₁-C₆ alkyl, C₃-C₇ cycloalkyl, and 3- to 7-membered heterocycloalkyl, wherein the C₁-C₆ alkyl, C₁-C₆ haloalkyl, C₃-C₇ cycloalkyl, and 3- to 7-membered heterocycloalkyl are optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 5-membered heterocycloalkyl, 5-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;
wherein each C₁-C₆ alkyl substituent and each C₁-C₆ alkoxy substituent of the R¹ or R² C₃-C₇ cycloalkyl or of the R¹ or R² 3- to 7-membered heterocycloalkyl is further optionally independently substituted with one to three hydroxy, halo, NR⁸R⁹, or oxo;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;
or one pair of R¹ and R² on adjacent atoms, taken together with the atoms connecting them, independently form one monocyclic or bicyclic C₄-C₁₂ carbocyclic ring or one monocyclic or bicyclic 5- to-12-membered heterocyclic ring containing 1-3 heteroatoms independently selected from O, N, and S, wherein the carbocyclic ring or heterocyclic ring is optionally independently substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR⁸R⁹, ═NR¹⁰, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR⁸R⁹, wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR⁸R⁹, ═NR¹⁰, COOC₁-C₆ alkyl, C₆-C₁₀ aryl, and CONR⁸R⁹.

In some embodiments of the compound of Formula AA, R¹ and R² are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO-C₆-C₁₀ aryl, CO(5- to 10-membered heteroaryl), CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₂-C₆ alkyl, N(C₁-C₆ alkyl)₂, NHCOC₁-C₆ alkyl, NHCOC₆-C₁₀ aryl, NHCO(5- to 10-membered heteroaryl), NHCO(3- to 7-membered heterocycloalkyl), NHCOC₂-C₆ alkynyl, NHCOOCC₁-C₆ alkyl, NH—(C═NR¹³)NR¹¹R¹², CONR⁸R⁹, SF₅, SC₁-C₆ alkyl, S(O₂)C₁-C₆ alkyl, S(O₂)_{NR}¹¹R¹², S(O)C₁-C₆ alkyl, C₃-C₇ cycloalkyl, and 3- to 7-membered heterocycloalkyl, wherein the C₁-C₆ alkyl, C₁-C₆ haloalkyl, C₃-C₇ cycloalkyl, and 3- to 7-membered heterocycloalkyl are optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 5-membered heterocycloalkyl, 5-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;
wherein each C₁-C₆ alkyl substituent and each C₁-C₆ alkoxy substituent of the R¹ or R² C₃-C₇ cycloalkyl or of the R¹ or R² 3- to 7-membered heterocycloalkyl is further optionally independently substituted with one to three hydroxy, halo, NR⁸R⁹, or oxo;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;

or one pair of R¹ and R² on adjacent atoms, taken together with the atoms connecting them, independently form one monocyclic or bicyclic C₄-C₁₂ carbocyclic ring or one monocyclic or bicyclic 5- to-12-membered heterocyclic ring containing 1-3 heteroatoms independently selected from O, N, and S, wherein the carbocyclic ring or heterocyclic ring is optionally independently substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR⁸R⁹, ═NR¹⁰, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR⁸R⁹, wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR⁸R⁹, ═NR¹⁰, COOC₁-C₆ alkyl, C₆-C₁₀ aryl, and CONR⁸R⁹.

The Groups R¹ and R²

In some embodiments,

R¹ and R² are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO-C₆-C₁₀ aryl, CO(5- to 10-membered heteroaryl), CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, S(O₂)NR¹¹R¹², S(O)C₁-C₆ alkyl, C₃-C₇ cycloalkyl and 3- to 7-membered heterocycloalkyl,

wherein the C₁-C₆ alkyl, C₁-C₆ haloalkyl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), and OCO(3- to 7-membered heterocycloalkyl);
wherein each C₁-C₆ alkyl substituent and each C₁-C₆ alkoxy substituent of the R¹ or R² C₃-C₇ cycloalkyl or of the R¹ or R² 3- to 7-membered heterocycloalkyl is further optionally independently substituted with one to three hydroxy, halo, NR⁸R⁹, or oxo;
wherein the 3- to 7-membered heterocycloalkyl, C₆-C₁₀ aryl, and 5- to 10-membered heteroaryl are optionally substituted with one or more substituents independently selected from halo, C₁-C₆ alkyl, and OC₁-C₆ alkyl;

or at least one pair of R¹ and R² on adjacent atoms, taken together with the atoms connecting them, independently form at least one C₄-C₈ carbocyclic ring or at least one 5- to-8-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹;

In some embodiments,

R¹ and R² are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO-C₆-C₁₀ aryl; CO(5- to 10-membered heteroaryl); CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, S(O₂)NR¹¹R¹², S(O)C₁-C₆ alkyl, C₃-C₇ cycloalkyl and 3- to 7-membered heterocycloalkyl,

wherein the C₁-C₆ alkyl, C₁-C₆ haloalkyl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;
wherein each C₁-C₆ alkyl substituent and each C₁-C₆ alkoxy substituent of the R¹ or R² C₃-C₇ cycloalkyl or of the R¹ or R² 3- to 7-membered heterocycloalkyl is further optionally independently substituted with one to three hydroxy, halo, NR⁸R⁹, or oxo;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;

or at least one pair of R¹ and R² on adjacent atoms, taken together with the atoms connecting them, independently form at least one C₄-C₈ carbocyclic ring or at least one 5- to 8-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹;

In some embodiments,

R¹ and R² are each independently selected from C₁-C₆ haloalkyl, C₁-C₆ alkoxy, C₁-C₆ haloalkoxy, halo, CN, NO₂, COC₁-C₆ alkyl, CO-C₆-C₁₀ aryl, CO(5- to 10-membered heteroaryl), CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, S(O₂)NR¹¹R¹², S(O)C₁-C₆ alkyl, C₃-C₇ cycloalkyl and 3- to 7-membered heterocycloalkyl, wherein the C₃-C₇ cycloalkyl, C₁-C₆ haloalkyl, and 3- to 7-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;
wherein each C₁-C₆ alkyl substituent and each C₁-C₆ alkoxy substituent of the R¹ or R² C₃-C₇ cycloalkyl or of the R¹ or R² 3- to 7-membered heterocycloalkyl is further optionally independently substituted with one to three hydroxy, halo, NR⁸R⁹, or oxo;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;

or at least one pair of R¹ and R² on adjacent atoms, taken together with the atoms connecting them, independently form at least one C₄-C₈ carbocyclic ring or at least one 5- to 8-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments,

R¹ and R² are each independently selected from C₁-C₆ alkyl, halo, CN, NO₂, COC₁-C₆ alkyl, CO-C₆-C₁₀ aryl, CO(5- to 10-membered heteroaryl), CO₂C₁-C₆ alkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, S(O₂)NR¹¹R¹², S(O)C₁-C₆ alkyl, C₃-C₇ cycloalkyl and 3- to 7-membered heterocycloalkyl,

wherein the C₁-C₆ alkyl, C₁-C₆ haloalkyl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;
wherein each C₁-C₆ alkyl substituent and each C₁-C₆ alkoxy substituent of the R¹ or R² C₃-C₇ cycloalkyl or of the R¹ or R² 3- to 7-membered heterocycloalkyl is further optionally independently substituted with one to three hydroxy, halo, NR⁸R⁹, or oxo;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;

or at least one pair of R¹ and R² on adjacent atoms, taken together with the atoms connecting them, independently form at least one C₄-C₈ carbocyclic ring or at least one 5- to 8-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments,

R¹ and R² are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO-C₆-C₁₀ aryl, CO(5- to 10-membered heteroaryl), CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, S(O₂)NR¹¹R¹², S(O)C₁-C₆ alkyl, C₃-C₇ cycloalkyl and 3- to 7-membered heterocycloalkyl,

wherein the C₁-C₆ alkyl, C₁-C₆ haloalkyl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;
wherein each C₁-C₆ alkyl substituent and each C₁-C₆ alkoxy substituent of the R¹ or R² C₃-C₇ cycloalkyl or of the R¹ or R² 3- to 7-membered heterocycloalkyl is further optionally independently substituted with one to three hydroxy, halo, or oxo;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;

or at least one pair of R¹ and R² on adjacent atoms, taken together with the atoms connecting them, independently form at least one C₄-C₈ carbocyclic ring or at least one 5- to-8-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments,

R¹ and R² are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO-C₆-C₁₀ aryl, CO(5- to 10-membered heteroaryl), CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, S(O₂)NR¹¹R¹², S(O)C₁-C₆ alkyl, C₃-C₇ cycloalkyl and 3- to 7-membered heterocycloalkyl,

wherein the C₁-C₆ alkyl, C₁-C₆ haloalkyl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl.

In some embodiments,

R¹ and R² are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO-C₆-C₁₀ aryl, CO(5- to 10-membered heteroaryl), CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, S(O₂)NR¹¹R¹², S(O)C₁-C₆ alkyl, C₃-C₇ cycloalkyl and 3- to 7-membered heterocycloalkyl,

wherein the C₁-C₆ alkyl, C₁-C₆ haloalkyl, C₃-C₇ cycloalkyl and 3- to 7-membered heterocycloalkyl are each unsubstituted;

or at least one pair of R¹ and R² on adjacent atoms, taken together with the atoms connecting them, independently form at least one C₄-C₈ carbocyclic ring or at least one 5- to-8-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹;

In some embodiments,

R¹ and R² are each independently selected from C₁-C₆ alkyl, halo, CN, COC₁-C₆ alkyl, CO₂C₁-C₆ alkyl, C₆-C₁₀ aryl, S(O)C₁-C₆ alkyl, 5- to 10-membered heteroaryl, and 3- to 7-membered heterocycloalkyl,

wherein the C₁-C₆ alkyl and 3- to 7-membered heterocycloalkyl is optionally substituted with one or more substituents each independently selected from hydroxy and oxo.

In some embodiments, m=1; n=0; and

R¹ is selected from C₁-C₆ alkyl, C₁-C₆ haloalkyl, C₁-C₆ alkoxy, C₁-C₆ haloalkoxy, halo, CN, NO₂, COC₁-C₆ alkyl, CO-C₆-C₁₀ aryl, CO(5- to 10-membered heteroaryl), CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, S(O₂)NR¹¹R¹², S(O)C₁-C₆ alkyl, C₃-C₇ cycloalkyl and 3- to 7-membered heterocycloalkyl,

wherein the C₁-C₆ alkyl, C₁-C₆ haloalkyl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl.

In some embodiments , m=1; n=0; and,

R¹ is selected from C₁-C₆ alkyl, halo, CN, COC₁-C₆ alkyl, CO₂C₁-C₆ alkyl, C₆-C₁₀ aryl, 5- to 10-membered heteroaryl, S(O)C₁-C₆ alkyl, and 3- to 7-membered heterocycloalkyl,

wherein the C₁-C₆ alkyl and 3- to 7-membered heterocycloalkyl is optionally substituted with one or more substituents each independently selected from hydroxy and oxo.

In some embodiments, m=1; n=1; and

R¹ and R² are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO-C₆-C₁₀ aryl, CO(5- to 10-membered heteroaryl), CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, S(O₂)NR¹¹R¹², S(O)C₁-C₆ alkyl, C₃-C₇ cycloalkyl and 3- to 7-membered heterocycloalkyl,

wherein the C₁-C₆ alkyl, C₁-C₆ haloalkyl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;

wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;

In some embodiments , m=1; n=1; and,

R¹ and R² are each independently selected from C₁-C₆ alkyl, halo, CN, COC₁-C₆ alkyl, CO₂C₁-C₆ alkyl, C₆-C₁₀ aryl, 5- to 10-membered heteroaryl, S(O)C₁-C₆ alkyl, and 3- to 7-membered heterocycloalkyl,

wherein the C₁-C₆ alkyl and 3- to 7-membered heterocycloalkyl is optionally substituted with one or more substituents each independently selected from hydroxy and oxo.

In some embodiments, m=1; n=1; and

R¹ and R² are on adjacent atoms, and taken together with the atoms connecting them, form a C₄-C₈ carbocyclic ring or a 5- to-8-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, m=1; n=1; and

R¹ and R² are on adjacent atoms, and taken together with the atoms connecting them, form a C₆ carbocyclic ring or a 5-to-6-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, m=1; n=1; and

R¹ and R² are on adjacent atoms, and taken together with the atoms connecting them, form a C₅ carbocyclic ring or a 5- to-6-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, COOC₁-C₆ alkyl, C₆-C₁₀ aryl, and CONR⁸R⁹.

In some embodiments, m=1; n=1; and

R¹ and R² are on adjacent atoms, and taken together with the atoms connecting them, form a C₄-C₈ carbocyclic ring or a 5- to 8-membered heterocyclic ring containing 1 or 2 heteroatoms independently selected from O, N, and S, wherein the carbocyclic ring or heterocyclic ring is unsubstituted.

In some embodiments, it is provided that:

(1) one or more of R¹ or R², when present, is selected from NR^{8′}R^{9′}, C(O)NR^{8′}R^{9′}, S(O)₂NR^{11′}R^{12′}, C(O)R^{13′}, C₁-C₆ alkyl, C₁-C₆ alkoxy, C₁-C₆ haloalkyl, C₃-C₇ cycloalkyl, 3- to 7-membered heterocycloalkyl, C₆-C₁₀ aryl, and 5- to 10-membered heteroaryl,

wherein each of the C₃-C₇ cycloalkyl, and 3- to 7-membered heterocycloalkyl is substituted with NR^{8′}R^{9′}, C(O)NR^{8′}R^{9′}, R^{15′}, C₁-C₆ haloalkoxy, or C₃-C₇ cycloalkyl;

wherein each of the C₁-C₆ alkyl and C₁-C₆ haloalkyl is substituted with NR^{8′}R^{9′}, C(O)NR^{8′}R^{9′}, R¹⁵, C₁-C₆ haloalkoxy, or C₃-C₇ cycloalkyl;

and

each of the C₁-C₆ alkoxy, C₆-C₁₀ aryl and 5- to 10-membered heteroaryl is substituted with one or more substituents each independently selected from hydroxy, halo, CN, oxo, C₁-C₆ alkyl, C₁-C₆ alkoxy, R¹⁵, C₁-C₆ haloalkoxy, NR⁸R⁹, ═NR¹⁰, COOC₁-C₆ alkyl, CONR⁸R⁹, 3- to 7-membered heterocycloalkyl, C₃-C₇ cycloalkyl, C₆-C₁₀ aryl, 5- to 10-membered heteroaryl, OCOC₁-C₆ alkyl, OCOC₆-C₁₀ aryl, OCO(5- to 10-membered heteroaryl), and OCO(3- to 7-membered heterocycloalkyl);

or

(2) one pair of R¹ and R² on adjacent atoms, taken together with the atoms connecting them, independently form one ring that is selected from:

(a) C₄-C₈ carbocyclic ring or 5- to-8-membered heterocyclic ring containing 1 or 2 heteroatoms independently selected from O, N, and S, wherein the carbocyclic ring or heterocyclic ring is independently substituted with one or more substituents each independently selected from from C₂-C₆ alkenyl, C₂-C₆ alkynyl, OC₃-C₁₀ cycloalkyl, CN, OS(O₂)C₆-C₁₀ aryl, S(O₂)C₆-C₁₀ aryl, 5- to 10-membered heteroaryl, C₃-C₁₀ cycloalkyl, and 3- to 10-membered heterocycloalkyl, wherein the S(O₂)C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR⁸R⁹, ═NR¹⁰, COOC₁-C₆ alkyl, C₆-C₁₀ aryl, and CONR⁸R⁹;

(b) C₄-C₈ carbocyclic ring or 5- to-8-membered heterocyclic ring containing 3 heteroatoms independently selected from O, N, and S, wherein the carbocyclic ring or heterocyclic ring is optionally independently substituted with one or more substituents independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR⁸R⁹, ═NR¹⁰, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR⁸R⁹, wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR⁸R⁹, ═NR¹⁰, COOC₁-C₆ alkyl, C₆-C₁₀ aryl, and CONR⁸R⁹; and

(c) monocyclic or bicyclic C₉-C₁₂ carbocyclic ring or monocyclic or bicyclic 9- to 12-membered heterocyclic ring containing 1-3 heteroatoms independently selected from O, N, and S, wherein the carbocyclic ring or heterocyclic ring is optionally independently substituted with one or more substituents independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR⁸R⁹, ═NR¹⁰, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR⁸R⁹, wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR⁸R⁹, ═NR¹⁰, COOC₁-C₆ alkyl, C₆-C₁₀ aryl, and CONR⁸R⁹;

each of R^{8′} and R^{9′} at each occurrence is independently selected from hydrogen, C₁-C₆ alkyl, C₃-C₇ cycloalkyl, C₂-C₆ alkenyl, (C═NR¹³)NR¹¹R¹², S(O₂)C₁-C₆ alkyl, S(O₂)NR¹¹R¹², COR¹³, CO₂R¹³ and CONR¹¹R¹²; wherein the C₁-C₆ alkyl is optionally substituted with one or more hydroxy, halo, C₁-C₆ alkoxy, C₆-C₁₀ aryl, 5- to 10-membered heteroaryl, C₃-C₇ cycloalkyl, 3- to 7-membered heterocycloalkyl, or NR¹¹R¹²;

or R^{8′} and R^{9′} taken together with the nitrogen they are attached to form a 3- to 10-membered monocyclic or bicyclic ring optionally containing one or more heteroatoms in addition to the nitrogen they are attached to, wherein the ring is optionally substituted with one or more substituents independently selected from halo, C₁-C₆ alkyl, C₁-C₆ haloalkyl, C₁-C₆ alkoxy, oxo, N(C₁-C₆alkyl)₂, NH₂, NH(C₁-C₆ alkyl), and hydroxy;

provided that:

(1) one or more occurrences of R^{8′} or R^{9′} is C₁-C₆ alkyl, C₂-C₆ alkenyl, C₃-C₇ cycloalkyl, (C═NR^{13′})NR^{11′}R^{12′}, S(O₂)NR^{11′}R^{12′}, C(O)R^{13′}, CO₂R^{13′} and CONR^{11′}R^{12′}; wherein the C₁-C₆ alkyl is substituted with NR¹¹R¹²; or

(2) one or more pairs of R^{8′} and R^{9′} attached to the same nitrogen taken together with the nitrogen they are attached to form:

(a) a 8- to 10-membered monocyclic or bicyclic ring optionally containing one or more heteroatoms in addition to the nitrogen they are attached to, wherein the ring is optionally substituted with one or more substituents independently selected from halo, C₁-C₆ alkyl, C₁-C₆ haloalkyl, C₁-C₆ alkoxy, oxo, N(C₁-C₆alkyl)₂, NH₂, NH(C₁-C₆ alkyl), and hydroxy; or

(b) a 3- to 7-membered monocyclic or bicyclic ring optionally containing one or more heteroatoms in addition to the nitrogen they are attached to, wherein the ring is substituted with one or more substituents independently selected from halo, C₁-C₆ alkyl, C₁-C₆ haloalkyl, C₁-C₆ alkoxy, oxo, N(C₁-C₆alkyl)₂, NH₂, NH(C₁-C₆ alkyl), and hydroxy;

each of R^{11′} and R^{12′} at each occurrence is independently selected from hydrogen, C₁-C₆ alkyl, and —(Z¹-Z²)ₐ₁-Z³, provided that one or more occurrences of R^{11′} and R^{12′} is —(Z¹-Z²)ₐ₁-Z³;

R^{13′} is —(Z¹-Z²)ₐ₁-Z^{3′}, wherein

when a1 is 0, Z^{3′} is independently C₆₋₁₀ aryl, C₃-C₁₀ cycloalkyl, 5- to 10-membered heteroaryl, 3- to 10-membered heterocycloalkyl, C₂-C₆ alkenyl, or C₂-C₆ alkynyl, each of which is substituted with one or more substituents independently selected from halo, C₁-C₆ alkyl, C₁₋₆ haloalkyl, C₁-C₆ alkoxy, oxo, N(C₁-C₆alkyl)₂, NH₂, NH(C₁-C₆ alkyl), and hydroxy;

when a1 is 1-10 (e.g., 1-4), Z^{3′} is an independently selected Z³;

R^{15′} is —(Z⁴-Z⁵)_{a2′}-Z⁶;

a2′ is an integer selected from 2-10 (e.g., 2-5);

each Z⁴ is independently selected from —O—, —S—, —NH—, and —N(C₁-C₃ alkyl)-;

provided that the Z⁴ group directly attached to R¹ or R² is —O— or —S—;

each Z⁵ is independently C₁-C₆ alkylene optionally substituted with one or more substituents independently selected from oxo, halo, and hydroxy; and

Z⁶ is OH, OC₁-C₆ alkyl, NH₂, NH(C₁-C₆ alkyl), N(C₁-C₆ alkyl)₂, NHC(O)(C₁-C₆ alkyl), NHC(O)(C₁-C₆ alkoxy), or an optionally substituted group selected from the group consisting of:

C₆-C₁₀ aryl, C₂-C₆ alkenyl, C₂-C₆ alkynyl, C₃-C₁₀ cycloalkyl, 5- to 10-membered heteroaryl, or 3- to 10-membered heterocycloalkyl, each of which is optionally substituted with one or more substituents independently selected from halo, C₁-C₆ alkyl, C₁₋₆ haloalkyl, C₁-C₆ alkoxy, oxo, N(C₁-C₆alkyl)₂, NH₂, NH(C₁-C₆ alkyl), and hydroxyl.

In some embodiments, R^{1′} is selected from the group consisting of:

methyl substituted with one or more substituents each independently selected from hydroxy, halo, CN, oxo, C₁-C₆ alkyl, C₁-C₆ alkoxy, R¹⁵, C₁-C₆ haloalkoxy, NR⁸R⁹, ═NR¹⁰, COOC₁-C₆ alkyl, CONR⁸R⁹, 3- to 7-membered heterocycloalkyl, C₃-C₇ cycloalkyl, C₆-C₁₀ aryl, 5- to 10-membered heteroaryl, OCOC₁-C₆ alkyl, OCOC₆-C₁₀ aryl, OCO(5- to 10-membered heteroaryl), and OCO(3- to 7-membered heterocycloalkyl);

C₂-C₆ alkyl; C₁-C₆ haloalkyl; C₁-C₆ alkoxy; C₁-C₆ haloalkoxy; halo; CN; NO₂; CO₂C₁-C₆ alkyl; CO₂C₃-C₈ cycloalkyl; OCOC₁-C₆ alkyl; OCOC₆-C₁₀ aryl; OCO(5- to 10-membered heteroaryl); OCO(3- to 7-membered heterocycloalkyl); C₆-C₁₀ aryl; 5- to 10-membered heteroaryl; NR⁸R⁹; C(O)R¹³; CONR⁸R⁹; SF₅; SC₁-C₆ alkyl; S(O₂)C₁-C₆ alkyl, S(O)C₁-C₆ alkyl; S(O₂)NR¹¹R¹²; C₃-C₇ cycloalkyl; and 3- to 7-membered heterocycloalkyl,

wherein the C₂-C₆ alkyl, C₁-C₆ alkoxy, C₁-C₆ haloalkyl, C₆-C₁₀ aryl, 5- to 10-membered heteroaryl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, R¹⁵, C₁-C₆ haloalkoxy, NR⁸R⁹, ═NR¹⁰, COOC₁-C₆ alkyl, CONR⁸R⁹, 3- to 7-membered heterocycloalkyl, C₃-C₇ cycloalkyl, C₆-C₁₀ aryl, 5- to 10-membered heteroaryl, OCOC₁-C₆ alkyl, OCOC₆-C₁₀ aryl, OCO(5- to 10-membered heteroaryl), and OCO(3- to 7-membered heterocycloalkyl); and

wherein each C₁-C₆ alkyl substituent and each C₁-C₆ alkoxy substituent of the R^{1′} C₃-C₇ cycloalkyl or of the R^{1′}3- to 7-membered heterocycloalkyl is further optionally independently substituted with one to three hydroxy, halo, —O(C₀-C₃ alkylene)C₆-C₁₀ aryl, NR⁸R⁹, or oxo;

wherein the 3- to 7-membered heterocycloalkyl, C₆-C₁₀ aryl, and 5- to 10-membered heteroaryl are optionally substituted with one or more substituents independently selected from halo, C₁-C₆ alkyl, and OC₁-C₆ alkyl.

In some embodiments, one pair of R¹ and R² is on adjacent atoms, and taken together with the atoms connecting them, independently form one monocyclic or bicyclic C₄-C₁₂ carbocyclic ring (e.g., C₅ or C₆ carbocyclic ring) or one monocyclic or bicyclic 5- to-12-membered heterocyclic ring containing 1-3 (e.g., 1-2, e.g., 2) heteroatoms independently selected from O, N, and S (e.g., tetrahydropyridine, dihydrofuran, or dihydropyran), wherein the carbocyclic ring or heterocyclic ring is optionally independently substituted with one or more substituents each independently selected from hydroxy, halo, oxo, C₁-C₆ alkyl (e.g., methyl), C₂-C₆ alkenyl, C₂-C₆ alkynyl, C₁-C₆ alkoxy (e.g., methoxy, ethoxy, isopropoxyl), OC₃-C₁₀ cycloalkyl, NR⁸R⁹, ═NR¹⁰, CN, COOC₁-C₆ alkyl, S(O₂)C₆-C₁₀ aryl, C₆-C₁₀ aryl, 5- to 10-membered heteroaryl, C₃-C₁₀ cycloalkyl, 3- to 10-membered heterocycloalkyl (e.g., azetidinyl or oxetanyl), and CONR⁸R⁹, wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo (e.g., fluoro), C₁-C₆ alkyl, C₂-C₆ alkenyl, C₂-C₆ alkynyl, C₃-C₁₀ cycloalkyl, C₁-C₆ alkoxy, oxo, NR⁸R⁹ (e.g., amino, methylamino, or dimethylamino), ═NR¹⁰, COOC₁-C₆ alkyl, C₆-C₁₀ aryl, and CONR⁸R⁹.

In some embodiments, one pair of R¹ and R² is on adjacent atoms, and taken together with the atoms connecting them, independently form one monocyclic or bicyclic C₅-C₆ carbocyclic ring,

wherein the carbocyclic is optionally independently substituted with one or more substituents each independently selected from hydroxy, halo, oxo, methyl, isopropoxyl, azetidinyl, oxetanyl, wherein the methyl, isopropoxyl, azetidinyl, and oxetanyl are optionally substituted with one or more substituents each independently selected from hydroxy, fluoro, amino, methylamino, and dimethylamino; or

one pair of R¹ and R² on adjacent atoms taken together forms a moiety selected from:

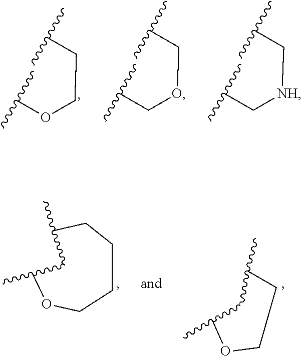

each of which is optionally independently substituted with one or more substituents each independently selected from hydroxy, halo, oxo, methyl, isopropoxyl, azetidinyl, oxetanyl, wherein the methyl, isopropoxyl, azetidinyl, and oxetanyl are optionally substituted with one or more substituents each independently selected from hydroxy, fluoro, amino, methylamino, and dimethylamino.

In some embodiments, one pair of R¹ and R² is on adjacent atoms, and taken together with the atoms connecting them, independently form at least one bicyclic spirocyclic C₄-C₁₂ carbocyclic ring, wherein the carbocyclic ring is optionally substituted with one or more substituents each independently selected from hydroxy, halo, oxo, methyl, isopropoxyl, azetidinyl, oxetanyl, wherein the methyl, isopropoxyl, azetidinyl, and oxetanyl are optionally substituted with one or more substituents each independently selected from hydroxy, fluoro, amino, methylamino, and dimethylamino.

In some embodiments, one pair of R¹ and R² is on adjacent atoms, and taken together with the atoms connecting them, independently form at least one bicyclic spirocyclic 5- to-12-membered heterocyclic ring containing 1-3 heteroatoms independently selected from O, N, and S, wherein the carbocyclic or heterocyclic ring is optionally substituted with one or more substituents each independently selected from hydroxy, halo, oxo, methyl, isopropoxyl, azetidinyl, oxetanyl, wherein the methyl, isopropoxyl, azetidinyl, and oxetanyl are optionally substituted with one or more substituents each independently selected from hydroxy, fluoro, amino, methylamino, and dimethylamino.

In some embodiments, each of R¹ and R², when present, is independently selected from the group consisting of C₁-C₆ alkyl optionally substituted with one or more hydroxy, halo, oxo, C₁-C₆ alkoxy, 3- to 7-membered heterocycloalkyl optionally substituted with halo, C₃-C₇ cycloalkyl, R¹⁵, C₁-C₆ haloalkoxy, C₆-C₁₀ aryl optionally substituted with OC₁-C₆ alkyl, or NR⁸R⁹; C₃-C₇ cycloalkyl optionally substituted with one or more hydroxy, halo, oxo, C₁-C₆ alkoxy, C₁-C₆ alkyl, or NR⁸R⁹ wherein the C₁-C₆ salkoxy or C₁-C₆ alkyl is further optionally substituted with one to three hydroxy, halo, NR⁸R⁹, or oxo; 3- to 7-membered heterocycloalkyl optionally substituted with one or more hydroxy, halo, oxo, C₁-C₆ alkyl, or NR⁸R⁹ wherein the C₁-C₆ alkoxy or C₁-C₆ alkyl is further optionally substituted with one to three hydroxy, halo, NR⁸R⁹, or oxo; C₁-C₆ haloalkyl; C₁-C₆ alkoxy; C₁-C₆ haloalkoxy; halo; CN; CO-C₁-C₆ alkyl; CO-C₆-C₁₀ aryl; CO(5- to 10-membered heteroaryl); CO₂C₁-C₆ alkyl; CO₂C₃-C₈ cycloalkyl; OCOC₁-C₆ alkyl; OCOC₆-C₁₀ aryl; OCO(5- to 10-membered heteroaryl); OCO(3- to 7-membered heterocycloalkyl); C₆-C₁₀ aryl; 5- to 10-membered heteroaryl; NH₂; NHC₁-C₆ alkyl; N(C₁-C₆ alkyl)₂; CONR⁸R⁹; SF₅; S(O₂)NR¹¹R¹²; S(O)C₁-C₆ alkyl; and S(O₂)C₁-C₆ alkyl.

In certain embodiments, R¹ is selected from the group consisting of 1-hydroxy-2-methylpropan-2-yl; methyl; isopropyl; isobutyl; difluoromethyl; 2-hydroxy-2-propyl; hydroxymethyl; 1-hydroxyethyl; 2-hydroxyethyl; 1-hydroxy-2-propyl; 1,2-dihydroxy-2-propyl; 1,2,3-trihydroxy-2-propyl; 1-amino-2-hydroxy-prop-2-yl; 1-acetamido-2-hydroxy-prop-2-yl; 1-(tert-butoxycarbonyl)amino-2-hydroxy-prop-2-yl; 1-(2-hydroxyethoxy)-2-hydroxy-2-propyl; 1-(2-benzyloxyethoxy)-2-hydroxy-2-propyl; 1-(2-methoxyethoxy)-2-hydroxy-2-propyl; 1-hydroxy-1-cyclopropyl; 1-hydroxy-1-cyclobutyl; 1-hydroxy-1-cyclopentyl; 1-hydroxy-1-cyclohexyl; morpholinyl; pyrrolidinyl; 1,3-dioxolan-2-yl; COCH₃; COCH₂CH₃; difluoromethoxy; 2-methoxy-2-propyl; (methylamino)methyl; (2,2-difluoroeth-1-yl)(methyl)aminomethyl; (2,2,2-trifluoroeth-1-yl)(methyl)aminomethyl; (dimethylamino)methyl; 1-(dimethylamino)ethyl; 2-((methyl)aminomethyl)-prop-2-yl; 2-((methyl)amino)-prop-2-yl; (methyl)(cyclopropylmethyl)aminomethyl; (methyl)(2-(dimethylamino)eth-1-yl)aminomethyl; (cyclobutyl)(methyl)aminomethyl; 1-(cyclobutyl)amino-eth-1-yl; isopropylaminomethyl; (cyclobutyl)aminomethyl; cycloheptylaminomethyl; tetrahydropyranylaminomethyl; sec-butylaminomethyl; ethylaminomethyl; allylaminomethyl; (2,2-difluoroeth-1-yl)aminomethyl; (2-methoxy-eth-1-yl)aminomethyl; (2-methoxy-eth-1-yl)(methyl)aminomethyl; 2-fluoro-1-dimethylamino-eth-1-yl; 1-dimethylamino-2,2-difluoroeth-1-yl; 1-dimethylamino-2,2,2-trifluoroeth-1-yl; 1-dimethylamino-2,2,2-trimethyleth-1-yl; dimethylamino(cyclopropyl)methyl; methoxymethyl; isopropyl(methyl)amino; fluoro; chloro; phenyl; pyridyl; pyrazolyl; azetidinyl; 5-methyl-oxazolidin-2-one-5-yl; S(O₂)CH₃; S(O₂)NR¹¹R¹²; (3,3-difluoropyrrolidin-1-yl)methyl; 1-(difluoromethoxyl)eth-1-yl; azetidinylmethyl; 1-((methyl)aminomethyl)-cycloprop-1-yl; 4-methoxybenzyl; 4-methyl-piperazin-1-yl; morpholinylmethyl; and cyclopentyl.

In certain of any of the foregoing embodiments, R² is selected from the group consisting of fluoro, chloro, cyano, methyl; methoxy; ethoxy; isopropyl; 1-hydroxy-2-methylpropan-2-yl; 2-hydroxy-2-propyl; hydroxymethyl; 1-hydroxyethyl; 2-hydroxyethyl; 1-hydroxy-2-propyl; 1-hydroxy-1-cyclopropyl; COCH₃; COPh; 2-methoxy-2-propyl; methoxymethyl; (dimethylamino)methyl; S(O₂)CH₃; and S(O₂)NR¹¹R¹².

In some embodiments, one or more R¹ when present is independently a C₁-C₆ alkyl substituted with one or more hydroxy.

In certain of these embodiments, one or more R¹ is independently selected from 1-hydroxy-2-methylpropan-2-yl; 2-hydroxy-2-propyl; hydroxymethyl; 1-hydroxyethyl; 2-hydroxyethyl; 1-hydroxy-2-propyl; 1,2-dihydroxy-2-propyl; and 1,2,3-trihydroxy-2-propyl.

In some embodiments, one or more R¹ when present is independently a C₁-C₆ alkyl substituted with one or more hydroxy and further substituted with one or more (e.g., one) NR⁸R⁹.

In certain of these embodiments, one or more R¹ is independently selected from 1-amino-2-hydroxy-prop-2-yl; 1-acetamido-2-hydroxy-prop-2-yl; and 1-(tert-butoxycarbonyl)amino-2-hydroxy-prop-2-yl.

In some embodiments, one or more R¹ when present is independently a C₁-C₆ alkyl substituted with one or more hydroxy and further substituted with one or more (e.g., one) R¹⁵.

In certain of these embodiments (e.g., a2=1 or 2), one or more R¹ is independently selected from 1-(2-hydroxyethoxy)-2-hydroxy-2-propyl; 1-(2-benzyloxyethoxy)-2-hydroxy-2-propyl; and 1-(2-methoxyethoxy)-2-hydroxy-2-propyl.

In certain of these embodiments (e.g., a2=1), one or more R¹ is independently selected from 1-(2-hydroxyethoxy)-2-hydroxy-2-propyl and 1-(2-methoxyethoxy)-2-hydroxy-2-propyl.

In certain embodiments (e.g., a2=1), one or more R¹ is independently selected from:

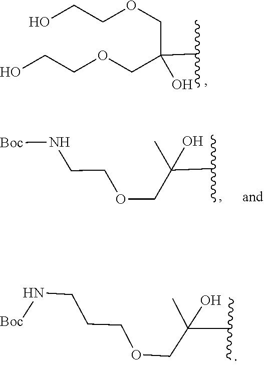 [SMILES: *C(O)(COCCO)COCCO ; *C(C)(O)COCCNC(=O)OC(C)(C)C ; *C(C)(O)COCCCNC(=O)OC(C)(C)C]

In certain embodiments (e.g., a2>1), one or more R¹ is

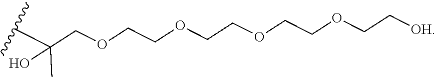 [SMILES: *C(C)(O)COCCOCCOCCOCCO]

In some embodiments, one or more R¹ is independently C₁-C₆ alkyl substituted with one or more (e.g., one) NR⁸R⁹ and further optionally substituted with one or more halo.

In certain of these embodiments, one or more R¹ is independently selected from: (methylamino)methyl; (2,2-difluoroeth-1-yl)(methyl)aminomethyl; (2,2,2-trifluoroeth-1-yl)(methyl)aminomethyl; (dimethylamino)methyl; 1-(dimethylamino)ethyl; 2-((methyl)aminomethyl)-prop-2-yl; 2-((methyl)amino)-prop-2-yl; (methyl)(cyclopropylmethyl)aminomethyl; (methyl)(2-(dimethylamino)eth-1-yl)aminomethyl; (cyclobutyl)(methyl)aminomethyl; 1-(cyclobutyl)amino-eth-1-yl; isopropylaminomethyl; (cyclobutyl)aminomethyl; cycloheptylaminomethyl; tetrahydropyranylaminomethyl; sec-butylaminomethyl; ethylaminomethyl; allylaminomethyl; (2,2-difluoroeth-1-yl)aminomethyl; (2-methoxy-eth-1-yl)aminomethyl; (2-methoxy-eth-1-yl)(methyl)aminomethyl; 2-fluoro-1-dimethylamino-eth-1-yl; 1-dimethylamino-2,2-difluoroeth-1-yl; 1-dimethylamino-2,2,2-trifluoroeth-1-yl; 1-dimethylamino-2,2,2-trimethyleth-1-yl; and dimethylamino(cyclopropyl)methyl (e.g., one or more R¹ is dimethylaminomethyl or methylaminomethyl).

In some embodiments, one or more R¹ is C₁-C₆ alkyl that is optionally substituted with one or more halo. In certain of these embodiments, one or more R¹ is C₂-C₆ alkyl that is optionally substituted with one or more halo. As non-limiting examples, R¹ is ethyl or difluoromethyl.

In certain of any of the foregoing embodiments of R¹, one or more R² is independently selected from C₁-C₆ alkyl, C₁-C₆ alkyl optionally substituted with one or more hydroxy, C₁-C₆ alkyl optionally substituted with one or more C₁-C₆ alkoxy, and halo.

Particular Embodiments wherein m=1 and n=0:

In some embodiments, R¹ is C₁-C₆ alkyl optionally substituted with one or more hydroxy.

In certain of these embodiments, R¹ is independently selected from 1-hydroxy-2-methylpropan-2-yl; 2-hydroxy-2-propyl; hydroxymethyl; 1-hydroxyethyl; 2-hydroxyethyl; 1-hydroxy-2-propyl; 1,2-dihydroxy-2-propyl; and 1,2,3-trihydroxy-2-propyl.

In some embodiments, R¹ is 1-hydroxy-2-methylpropan-2-yl.

In some embodiments, R¹ is 2-hydroxy-2-propyl.

In some embodiments, R¹ is hydroxymethyl.

In some embodiments, R¹ is 1-hydroxyethyl.

In some embodiments, R¹ is 1-hydroxy-2-propyl.

In some embodiments, R¹ is 2-hydroxyethyl.

In some embodiments, R¹ is 1,2-dihydroxy-2-propyl.

In some embodiments, R¹ is 1,2,3-trihydroxy-2-propyl.

In some embodiments, R¹ is C₁-C₆ alkyl.

In some embodiments, R¹ is isobutyl.

In some embodiments, R¹ is methyl.

In some embodiments, R¹ is isopropyl.

In some embodiments, R¹ is isopropyl.

In some embodiments, R¹ is C₁-C₆ alkyl substituted with hydroxy at the carbon directly connected to ring A.

In some embodiments, R¹ is 2-hydroxy-2-propyl.

In some embodiments, R¹ is hydroxymethyl.

In some embodiments, R¹ is 1-hydroxyethyl.

In some embodiments, R¹ is 1-hydroxy-2-propyl.

In some embodiments, R¹ is C₁-C₆ alkyl substituted with two or more hydroxy groups.

In some embodiments, R¹ is C₁-C₆ alkyl substituted with two or more hydroxy groups, wherein one of the two or more hydroxy groups is bonded to the carbon directly connected to ring A.

In some embodiments, R¹ is 1,2-dihydroxy-prop-2-yl.

In some embodiments, R¹ is a C₁-C₆ alkyl substituted with one or more hydroxy and further substituted with one or more (e.g., one) NR⁸R⁹.

In certain of these embodiments, R¹ is independently selected from 1-amino-2-hydroxy-prop-2-yl; 1-acetamido-2-hydroxy-prop-2-yl; and 1-(tert-butoxycarbonyl)amino-2-hydroxy-prop-2-yl.

In some embodiments, R¹ is 1-amino-2-hydroxy-prop-2-yl.

In some embodiments, R¹ is 1-acetamido-2-hydroxy-prop-2-yl.

In some embodiments, R¹ is 1-(tert-butoxycarbonyl)amino-2-hydroxy-prop-2-yl.

In some embodiments, R¹ is independently a C₁-C₆ alkyl substituted with one or more hydroxy and further substituted with one or more (e.g., one) R¹⁵.

In certain of these embodiments, a2 is 1 in R¹⁵.

In certain of the foregoing embodiments, one or more R¹ is independently selected from 1-(2-hydroxyethoxy)-2-hydroxy-2-propyl; 1-(2-benzyloxyethoxy)-2-hydroxy-2-propyl; and 1-(2-methoxyethoxy)-2-hydroxy-2-propyl.

In certain embodiments, R¹ is

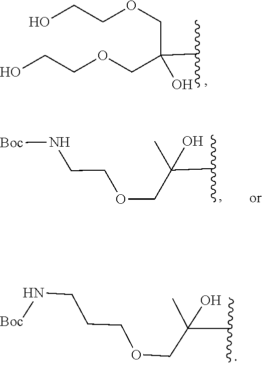 [SMILES: *C(O)(COCCO)COCCO ; *C(C)(O)COCCNC(=O)OC(C)(C)C ; *C(C)(O)COCCCNC(=O)OC(C)(C)C]

In certain embodiments when R¹ is independently a C₁-C₆ alkyl substituted with one or more hydroxy and further substituted with one or more (e.g., one) R¹⁵, a2 is >1.

In certain of these embodiments, R¹ is:

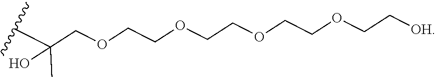 [SMILES: *C(C)(O)COCCOCCOCCOCCO]

In some embodiments, R¹ is C₁-C₆ alkyl optionally substituted with one or more R¹⁵.

In some embodiments, R¹ is C₃-C₇ cycloalkyl optionally substituted with one or more hydroxy.

In some embodiments, R¹ is C₃-C₇ cycloalkyl (e.g., cyclopropyl or cyclopentyl).

In some embodiments, R¹ is C₃-C₇ cycloalkyl optionally substituted with one or more C₁-C₆ alkyl wherein said C₁-C₆ alkyl is further optionally substituted as described elsewhere herein.

In some embodiments, R¹ is 1-((methyl)aminomethyl)-cycloprop-1-yl.

In some embodiments, R¹ is C₃-C₇ cycloalkyl substituted with hydroxy at the carbon directly connected to ring A.

In some embodiments, R¹ is 1-hydroxy-1-cyclopropyl.

In some embodiments, R¹ is 1-hydroxy-1-cyclobutyl.

In some embodiments, R^{e} is 1-hydroxy-1-cyclopentyl.

In some embodiments, R¹ is 1-hydroxy-1-cyclohexyl.

In some embodiments, R¹ is 3- to 7-membered heterocycloalkyl optionally substituted with one or more hydroxy.

In some embodiments, R¹ is 3- to 7-membered heterocycloalkyl.

In some embodiments, R¹ is morpholinyl (e.g., 1-morpholinyl).

In some embodiments, R¹ is azetidinyl.

In some embodiments, R¹ is 1,3-dioxolan-2-yl.

In some embodiments, R¹ is 3- to 7-membered heterocycloalkyl optionally substituted with one or more C₁-C₆ alkyl.

In some embodiments, R¹ is 1-methylpyrrolidin-2-yl.

In some embodiments, R¹ is 3- to 7-membered heterocycloalkyl substituted with hydroxy at the carbon directly connected to ring A.

In some embodiments, R¹ is C₁-C₆ alkyl optionally substituted with one or more oxo.

In some embodiments, R¹ is COCH₃.

In some embodiments, R¹ is COCH₂CH₃.

In some embodiments, R¹ is C₃-C₇ cycloalkyl optionally substituted with one or more oxo.

In some embodiments, R¹ is 3- to 7-membered heterocycloalkyl optionally substituted with one or more oxo.

In some embodiments, R¹ is C₁-C₆ alkyl optionally substituted with one or more C₁-C₆ alkoxy.

In some embodiments, R¹ is 2-methoxy-2-propyl.

In some embodiments, R¹ is methoxymethyl.

In some embodiments, R¹ is C₁-C₆ alkyl optionally substituted with one or more C₁-C₆ haloalkoxy.

In some embodiments, R¹ is 1-(difluoromethoxyl)eth-1-yl.

In some embodiments, R¹ is C₃-C₇ cycloalkyl optionally substituted with one or more C₁-C₆ alkoxy.

In some embodiments, R¹ is 3- to 7-membered heterocycloalkyl optionally substituted with one or more C₁-C₆ alkoxy.

In some embodiments, R¹ is 3- to 7-membered heterocycloalkyl optionally substituted with one or more oxo and further optionally substituted with one or more C₁-C₆ alkyl.

In some embodiments, R¹ is 5-methyl-oxazolidin-2-one-5-yl.

In some embodiments, R¹ is C₁-C₆ alkyl optionally substituted with one or more NR⁸R⁹.

In some embodiments, R¹ is C₁-C₆ alkyl substituted with NR⁸R⁹ at the carbon directly connected to ring A.

In some embodiments, R¹ is (methylamino)methyl.

In some embodiments, R¹ is (dimethylamino)methyl.

In some embodiments, R¹ is aminomethyl.

In some embodiments, R¹ is N-methylacetamidomethyl.

In some embodiments, R¹ is 1-(dimethylamino)eth-1-yl.

In some embodiments, R¹ is 2-(dimethylamino)prop-2-yl.

In some embodiments, R¹ is (2-methoxy-eth-1-yl)(methyl)aminomethyl.

In some embodiments, R¹ is (methyl)(acetyl)aminomethyl.

In some embodiments, R¹ is (methyl)(cyclopropylmethyl)aminomethyl.

In some embodiments, R¹ is (methyl)(2,2-difluoroeth-1-yl)aminomethyl.

In some embodiments, R¹ is (2,2,2-trifluoroeth-1-yl)(methyl)aminomethyl.

In some embodiments, R¹ is 2-((methyl)aminomethyl)-prop-2-yl.

In some embodiments, R¹ is 2-((methyl)amino)-prop-2-yl.

In some embodiments, R¹ is (methyl)(cyclopropylmethyl)aminomethyl.

In some embodiments, R¹ is (methyl)(2-(dimethylamino)eth-1-yl)aminomethyl.

In some embodiments, R¹ is (cyclobutyl)(methyl)aminomethyl.

In some embodiments, R¹ is (2-methoxy-eth-1-yl)(methyl)aminomethyl.

In some embodiments, R¹ is 2-fluoro-1-dimethylamino-eth-1-yl.

In some embodiments, R¹ is 1-dimethylamino-2,2-difluoroeth-1-yl.

In some embodiments, R¹ is 1-dimethylamino-2,2,2-trifluoroeth-1-yl.

In some embodiments, R¹ is 1-dimethylamino-2,2,2-trimethyleth-1-yl.

In some embodiments, R¹ is (cyclobutyl)(methyl)aminomethyl.

In some embodiments, R¹ is isopropylaminomethyl.

In some embodiments, R¹ is (cyclobutyl)aminomethyl.

In some embodiments, R¹ is cycloheptylaminomethyl.

In some embodiments, R¹ is tetrahydropyranylaminomethyl.

In some embodiments, R¹ is sec-butylaminomethyl.

In some embodiments, R¹ is ethylaminomethyl.

In some embodiments, R¹ is allylaminomethyl.

In some embodiments, R¹ is 2,2-difluoroeth-1-yl)aminomethyl.

In some embodiments, R¹ is (2-methoxy-eth-1-yl)aminomethyl.

In some embodiments, R¹ is C₁-C₆ alkyl substituted with NR⁸R⁹, wherein said C₁-C₆ alkyl is further optionally substituted as described elsewhere herein.

In some embodiments, R¹ is dimethylamino(cyclopropyl)methyl.

In some embodiments, R¹ is C₃-C₇ cycloalkyl optionally substituted with one or more NR⁸R⁹.

In some embodiments, R¹ is 3- to 7-membered heterocycloalkyl optionally substituted with one or more NR⁸R⁹.

In some embodiments, R¹ is C₁-C₆ haloalkyl optionally substituted with one or more hydroxy.

In some embodiments, R¹ is C₁-C₆ alkoxy that is optionally substituted as defined elsewhere herein.

In some embodiments, R¹ is C₁-C₆ alkoxy.

In some embodiments, R¹ is C₁-C₆ haloalkoxy.

In some embodiments, R¹ is difluoromethoxy.

In some embodiments, R¹ is C₁-C₆ alkyl optionally substituted with 3- to 7-membered heterocycloalkyl, wherein the 3- to 7-membered heterocycloalkyl is further optionally substituted as defined elsewhere herein.

In some embodiments, R¹ is pyrrolidinylmethyl (e.g., pyrrolidin-1-ylmethyl).

In some embodiments, R¹ is optionally substituted pyrrolidinylmethyl (e.g., 3,3-difluoropyrrolidin-1-ylmethyl).

In some embodiments, R¹ is azetidinylmethyl (e.g., azetidin-1-ylmethyl).

In some embodiments, R¹ is optionally substituted azetidinylmethyl (e.g., 3-methoxyazetidin-1-ylmethyl).

In some embodiments, R¹ is morpholinylmethyl (e.g., morpholin-4-ylmethyl).

In some embodiments, R¹ is 4-methyl-piperazin-1-yl.

In some embodiments, R¹ is 3- to 7-membered heterocycloalkyl optionally substituted with one or more hydroxy. In certain of these embodiments, R¹ is further optionally substituted with one or more C₁-C₆ alkyl, wherein each of said C₁-C₆ alkyl is further optionally substituted as defined anywhere herein. As a non-limiting example, R¹ is

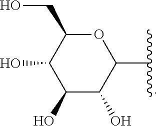 [SMILES: *C1O[C@H](CO)[C@@H](O)[C@H](O)[C@H]1O]

In some embodiments, R¹ is 3- to 7-membered heterocycloalkyl optionally substituted with one or more substituents independently selected from hydroxy and R¹⁵. In certain of these embodiments, R¹ is further optionally substituted with one or more C₁-C₆ alkyl, wherein each of said C₁-C₆ alkyl is further optionally substituted as defined anywhere herein. As a non-limiting example, R¹ is

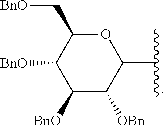 [SMILES: *C1O[C@H](COCc2ccccc2)[C@@H](OCc2ccccc2)[C@H](OCc2ccccc2)[C@H]1OCc1ccccc1]

In some embodiments, R¹ is C₁-C₆ alkyl optionally substituted with one or more C₆-C₁₀ aryl, wherein said aryl is further optionally substituted as described elsewhere herein.

In some embodiments, R¹ is 4-methoxybenzyl.

In some embodiments, R¹ is halo.

In some embodiments, R¹ is fluoro.

In some embodiments, R¹ is chloro.

In some embodiments, R¹ is CN.

In some embodiments, R¹ is NO₂.

In some embodiments, R¹ is COC₁-C₆ alkyl.

In some embodiments, R¹ is CO-C₆-C₁₀ aryl.

In some embodiments, R¹ is CO(5- to 10-membered heteroaryl).

In some embodiments, R¹ is CO₂C₁-C₆ alkyl.

In some embodiments, R¹ is CO₂C₃-C₈ cycloalkyl.

In some embodiments, R¹ is OCOC₁-C₆ alkyl.

In some embodiments, R¹ is OCOC₆-C₁₀ aryl.

In some embodiments, R¹ is OCO(5- to 10-membered heteroaryl).

In some embodiments, R¹ is OCO(3- to 7-membered heterocycloalkyl).

In some embodiments, R¹ is C₆-C₁₀ aryl.

In some embodiments, R¹ is phenyl.

In some embodiments, R¹ is 5- to 10-membered heteroaryl.

In some embodiments, R¹ is pyridyl (e.g., 4-pyridyl).

In some embodiments, R¹ is pyrazolyl (e.g., 1-pyrazolyl).

In some embodiments, R¹ is NH₂.

In some embodiments, R¹ is NHC₁-C₆ alkyl.

In some embodiments, R¹ is N(C₁-C₆ alkyl)₂.

In some embodiments, R¹ is CONR⁸R⁹.

In some embodiments, R¹ is SF₅.

In some embodiments, R¹ is SC₁-C₆ alkyl,

In some embodiments, R¹ is S(O₂)C₁-C₆ alkyl.

In some embodiments, R¹ is S(O₂)CH₃.

In some embodiments, R¹ is S(O₂)NR¹¹R¹².

In some embodiments, R¹ is S(O₂)N(CH₃)₂.

In some embodiments, R¹ is S(O)C₁-C₆ alkyl.

In some embodiments, R¹ is S(O)CH₃.

In some embodiments, R¹ is attached to a carbon of an aryl ring A.

In some embodiments, R¹ is attached to a carbon of a heteroaryl ring A.

In some embodiments, R¹ is attached to a nitrogen of a heteroaryl ring A.

Particular Embodiments wherein m=1 and n=1:

In some embodiments, R¹ is C₁-C₆ alkyl optionally substituted with one or more hydroxy, and R² is C₁-C₆ alkyl optionally substituted with one or more hydroxy.

In some embodiments, R¹ is 1-hydroxy-2-methylpropan-2-yl, and R² is methyl.

In some embodiments, R¹ is 2-hydroxy-2-propyl and R² is methyl.

In some embodiments, R¹ is 2-hydroxy-2-propyl and R² is isopropyl.

In some embodiments, R¹ is 2-hydroxy-2-propyl and R² is 2-hydroxy-2-propyl.

In some embodiments, R¹ is 2-hydroxy-2-propyl and R² is 1-hydroxyethyl.

In some embodiments, R¹ is hydroxymethyl and R² is methyl.

In some embodiments, R¹ is 1-hydroxyethyl and R² is methyl.

In some embodiments, R¹ is 2-hydroxyethyl and R² is methyl.

In some embodiments, R¹ is 1-hydroxy-2-propyl and R² is methyl.

In some embodiments, R¹ is C₁-C₆ alkyl optionally substituted with one or more hydroxy, and R² is C₆-C₁₀ aryl.

In some embodiments, R¹ is 2-hydroxy-2-propyl and R² is phenyl.

In some embodiments, R¹ is C₁-C₆ alkyl optionally substituted with one or more hydroxy, and R² is 5- to 10-membered heteroaryl.

In some embodiments, R¹ is 2-hydroxy-2-propyl and R² is pyridyl.

In some embodiments, R¹ is 2-hydroxy-2-propyl and R² is pyrazolyl.

In some embodiments, R¹ is C₁-C₆ alkyl optionally substituted with one or more hydroxy, and R² is SF₅.

In some embodiments, R¹ is C₁-C₆ alkyl optionally substituted with one or more hydroxy, and R² is SC₁-C₆ alkyl,

In some embodiments, R¹ is C₁-C₆ alkyl optionally substituted with one or more hydroxy, and R² is S(O₂)C₁-C₆ alkyl.

In some embodiments, R¹ is C₁-C₆ alkyl optionally substituted with one or more hydroxy, and R² is S(O₂)CH₃.

In some embodiments, R¹ is C₁-C₆ alkyl optionally substituted with one or more hydroxy, and R² is halo.

In some embodiments, R¹ is 2-hydroxy-2-propyl and R² is chloro.

In some embodiments, R¹ is 2-hydroxy-2-propyl and R² is fluoro.

In some embodiments, R¹ is 1,2-dihydroxy-2-propyl and R² is fluoro.

In some embodiments, R¹ is 1,2-dihydroxy-2-propyl and R² is chloro.

In some embodiments, R¹ is C₁-C₆ alkyl optionally substituted with one or more hydroxy, and R² is C₁-C₆ alkyl optionally substituted with one or more C₁-C₆ alkoxy.

In some embodiments, R¹ is 2-hydroxy-2-propyl and R² is methoxymethyl.

In some embodiments, R¹ is C₃-C₇ cycloalkyl optionally substituted with one or more hydroxy, and R² is C₁-C₆ alkyl.

In some embodiments, R¹ is 1-hydroxy-1-cyclopropyl, and R² is methyl.

In some embodiments, R¹ is 1-hydroxy-1-cyclobutyl, and R² is methyl.

In some embodiments, R¹ is 1-hydroxy-1-cyclopentyl, and R² is methyl.

In some embodiments, R¹ is 1-hydroxy-1-cyclohexyl, and R² is methyl.

In some embodiments, R¹ is 3- to 7-membered heterocycloalkyl optionally substituted with one or more hydroxy, and R² is C₁-C₆ alkyl.

In some embodiments, R¹ is morpholinyl, and R² is methyl.

In some embodiments, R¹ is 1,3-dioxolan-2-yl, and R² is methyl.

In some embodiments, R¹ is 3- to 7-membered heterocycloalkyl optionally substituted with one or more hydroxy, and R² is halo.

In some embodiments, R¹ is 1,3-dioxolan-2-yl, and R² is fluoro.

In some embodiments, R¹ is 1,3-dioxolan-2-yl, and R² is chloro.

In some embodiments, R¹ is C₁-C₆ alkyl optionally substituted with one or more oxo, and R² is methyl.

In some embodiments, R¹ is COCH₃, and R² is methyl.

In some embodiments, R¹ is C₁-C₆ alkyl optionally substituted with one or more C₁-C₆ alkoxy, and R² is C₁-C₆ alkyl.

In some embodiments, R¹ is 2-methoxy-2-propyl, and R² is methyl.

In some embodiments, R¹ is C₁-C₆ alkyl optionally substituted with one or more NR⁸R⁹, and R² is C₁-C₆ alkyl.

In some embodiments, R¹ is (dimethylamino)methyl, and R² is methyl.

In some embodiments, R¹ is C₁-C₆ alkyl optionally substituted with one or more NR⁸R⁹, and R² is halo.

In some embodiments, R¹ is (dimethylamino)methyl, and R² is fluoro.

In some embodiments, R¹ is (dimethylamino)methyl, and R² is fluoro.

In some embodiments, R¹ is (methylamino)methyl, and R² is fluoro.

In some embodiments, R¹ is aminomethyl, and R² is fluoro.

In some embodiments, R¹ is C₁-C₆ alkyl, and R² is C₁-C₆ alkyl.

In some embodiments, R¹ is methyl, and R² is methyl.

In some embodiments, R¹ is C₁-C₆ alkyl substituted with one or more hydroxy; and R² is C₁-C₆ alkyl substituted with one or more hydroxy. In certain of the foregoing embodiments, R¹ or R² is further optionally substituted as defined elsewhere herein (e.g., R¹ or R² is further optionally substituted with one R¹⁵).

In some embodiments, R¹ is C₁-C₆ alkyl substituted with one or more hydroxy; and R² is hydroxymethyl.

In some embodiments, R¹ is 1,3-dihydroxy-2-methyl-2-propyl; and R² is hydroxymethyl.

In some embodiments, R¹ is 2-hydroxymethyl-2-propyl; and R² is hydroxymethyl.

In some embodiments, R¹ is 2-hydroxyeth-1-yl; and R² is hydroxymethyl.

In some embodiments, R¹ is 1,2-dihydroxy-3-propyl; and R² is hydroxymethyl.

In some embodiments, R¹ is 1,2,3-trihydroxy-2-propyl; and R² is hydroxymethyl.

In some embodiments, R¹ is 2-hydroxy-2-propyl; and R² is hydroxymethyl.

In some embodiments, R¹ is 1,2-dihydroxy-2-propyl; and R² is hydroxymethyl.

In some embodiments, R¹ is:

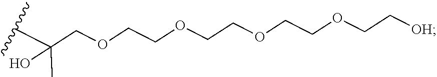 [SMILES: *C(C)(O)COCCOCCOCCOCCO]

and R² is hydroxymethyl.

In some embodiments, R² is C₁-C₆ alkyl substituted with one or more hydroxy; and R¹ is hydroxymethyl.

In some embodiments, R² is 1,3-dihydroxy-2-methyl-2-propyl; and R¹ is hydroxymethyl.

In some embodiments, R² is 2-hydroxymethyl-2-propyl; and R¹ is hydroxymethyl.

In some embodiments, R² is 2-hydroxyeth-1-yl; and R¹ is hydroxymethyl.

In some embodiments, R² is 1,2-dihydroxy-3-propyl; and R¹ is hydroxymethyl.

In some embodiments, R² is 1,2,3-trihydroxy-2-propyl; and R¹ is hydroxymethyl.

In some embodiments, R² is 2-hydroxy-2-propyl; and R¹ is hydroxymethyl.

In some embodiments, R² is 1,2-dihydroxy-2-propyl; and R¹ is hydroxymethyl.

In some embodiments, R² is:

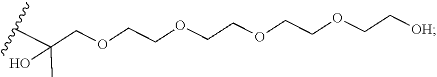 [SMILES: *C(C)(O)COCCOCCOCCOCCO]

and R¹ is hydroxymethyl.

In some embodiments, R² is 1-hydroxy-2-methylpropan-2-yl, and R¹ is methyl.

In some embodiments, R² is 2-hydroxy-2-propyl and R¹ is methyl.

In some embodiments, R² is 2-hydroxy-2-propyl and R¹ is isopropyl.

In some embodiments, R² is 2-hydroxy-2-propyl and R¹ is 1-hydroxyethyl.

In some embodiments, R² is hydroxymethyl and R¹ is methyl.

In some embodiments, R² is 1-hydroxyethyl and R¹ is methyl.

In some embodiments, R² is 2-hydroxyethyl and R¹ is methyl.

In some embodiments, R² is 1-hydroxy-2-propyl and R¹ is methyl.

In some embodiments, R² is C₁-C₆ alkyl optionally substituted with one or more hydroxy, and R¹ is C₆-C₁₀ aryl.

In some embodiments, R² is 2-hydroxy-2-propyl and R¹ is phenyl.

In some embodiments, R² is C₁-C₆ alkyl optionally substituted with one or more hydroxy, and R¹ is 5- to 10-membered heteroaryl.

In some embodiments, R² is 2-hydroxy-2-propyl and R¹ is pyridyl.

In some embodiments, R² is 2-hydroxy-2-propyl and R¹ is pyrazolyl.

In some embodiments, R² is C₁-C₆ alkyl optionally substituted with one or more hydroxy, and R¹ is SF₅.

In some embodiments, R² is C₁-C₆ alkyl optionally substituted with one or more hydroxy, and R¹ is SC₁-C₆ alkyl.

In some embodiments, R² is C₁-C₆ alkyl optionally substituted with one or more hydroxy, and R¹ is S(O₂)C₁-C₆ alkyl.

In some embodiments, R² is C₁-C₆ alkyl optionally substituted with one or more hydroxy, and R¹ is S(O₂)CH₃.

In some embodiments, R² is C₁-C₆ alkyl optionally substituted with one or more hydroxy, and R¹ is halo.

In some embodiments, R² is 2-hydroxy-2-propyl and R¹ is chloro.

In some embodiments, R² is 2-hydroxy-2-propyl and R¹ is fluoro.

In some embodiments, R² is 1,2-dihydroxy-2-propyl and R¹ is fluoro.

In some embodiments, R² is 1,2-dihydroxy-2-propyl and R¹ is chloro.

In some embodiments, R¹ is C₁-C₆ alkyl optionally substituted with one or more hydroxy, and R² is C₁-C₆ alkyl optionally substituted with one or more C₁-C₆ alkoxy.

In some embodiments, R¹ is 2-hydroxy-2-propyl and R² is methoxymethyl.

In some embodiments, R² is C₃-C₇ cycloalkyl optionally substituted with one or more hydroxy, and R¹ is C₁-C₆ alkyl.

In some embodiments, R² is 1-hydroxy-1-cyclopropyl, and R¹ is methyl.

In some embodiments, R² is 1-hydroxy-1-cyclobutyl, and R¹ is methyl.

In some embodiments, R² is 1-hydroxy-1-cyclopentyl, and R¹ is methyl.

In some embodiments, R² is 1-hydroxy-1-cyclohexyl, and R¹ is methyl.

In some embodiments, R² is 3- to 7-membered heterocycloalkyl optionally substituted with one or more hydroxy, and R¹ is C₁-C₆ alkyl.

In some embodiments, R² is morpholinyl, and R¹ is methyl.

In some embodiments, R² is 1,3-dioxolan-2-yl, and R¹ is methyl.

In some embodiments, R² is 3- to 7-membered heterocycloalkyl optionally substituted with one or more hydroxy, and R¹ is halo.

In some embodiments, R² is 1,3-dioxolan-2-yl, and R¹ is fluoro.

In some embodiments, R² is 1,3-dioxolan-2-yl, and R¹ is chloro.

In some embodiments, R² is C₁-C₆ alkyl optionally substituted with one or more oxo, and R¹ is methyl.

In some embodiments, R² is COCH₃, and R¹ is methyl.

In some embodiments, R² is C₁-C₆ alkyl optionally substituted with one or more C₁-C₆ alkoxy, and R¹ is C₁-C₆ alkyl.

In some embodiments, R² is 2-methoxy-2-propyl, and R¹ is methyl.

In some embodiments, R² is C₁-C₆ alkyl optionally substituted with one or more NR⁸R⁹, and R¹ is C₁-C₆ alkyl.

In some embodiments, R² is (dimethylamino)methyl, and R¹ is methyl.

In some embodiments, R² is C₁-C₆ alkyl optionally substituted with one or more NR⁸R⁹, and R¹ is halo.

In some embodiments, R² is (dimethylamino)methyl, and R¹ is fluoro.

In some embodiments, R² is (methylamino)methyl, and R¹ is fluoro.

In some embodiments, R² is aminomethyl, and R¹ is fluoro.

In some embodiments, R² is C₁-C₆ alkoxy, and R¹ is C₁-C₆ alkyl optionally substituted with one or more NR⁸R⁹.

In some embodiments, R² is methoxy, and R¹ is (dimethylamino)methyl.

In some embodiments, R¹ and R² are each attached to a carbon of an aryl ring A.

In some embodiments, R¹ and R² are each attached to a carbon of a heteroaryl ring A.

In some embodiments, R¹ is attached to a carbon and R² is attached to a nitrogen of a heteroaryl ring A.

In some embodiments, R² is attached to a carbon and R¹ is attached to a nitrogen of a heteroaryl ring A.

In some embodiments, R¹ and R² are on adjacent atoms, and taken together with the atoms connecting them, form a C₅ carbocyclic ring optionally substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, R¹ and R² are on adjacent atoms, and taken together with the atoms connecting them, form a C₅ aliphatic carbocyclic ring.

In some embodiments, R¹ and R² are on adjacent atoms, and taken together with the atoms connecting them, form a C₆ carbocyclic ring optionally substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, R¹ and R² are on adjacent atoms, and taken together with the atoms connecting them, form a C₆ aliphatic carbocyclic ring.

In some embodiments, R¹ and R² are on adjacent atoms, and taken together with the atoms connecting them, form a C₆ aromatic carbocyclic ring.

In some embodiments, R¹ and R² are on adjacent atoms, and taken together with the atoms connecting them, form a 5-membered heterocyclic ring containing 1 or 2 heteroatoms independently selected from O, N, and S, optionally substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, R¹ and R² are on adjacent atoms, and taken together with the atoms connecting them, form a 5-membered aliphatic heterocyclic ring containing 1 or 2 heteroatoms independently selected from O, N, and S.

In some embodiments, R¹ and R² are on adjacent atoms, and taken together with the atoms connecting them, form a 5-membered heteroaromatic ring containing 1 or 2 heteroatoms independently selected from O, N, and S.

In some embodiments, R¹ and R² are on adjacent atoms, and taken together with the atoms connecting them, form a 6-membered heterocyclic ring containing 1 or 2 heteroatoms independently selected from O, N, and S, optionally substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, R¹ and R² are on adjacent atoms, and taken together with the atoms connecting them, form a 6-membered aliphatic heterocyclic ring containing 1 or 2 heteroatoms independently selected from O, N, and S.

In some embodiments, R¹ and R² are on adjacent atoms, and taken together with the atoms connecting them, form a 6-membered heteroaromatic ring containing 1 or 2 heteroatoms independently selected from O, N, and S.

In some embodiments, R¹ and R² are different.

In some embodiments, R¹ and R² are different, and R² comprises a carbonyl group.

In some embodiments, R¹ and R² are different, and R² comprises 1 or 2 (e.g., 1) nitrogen atoms.

In some embodiments, R¹ and R² are different, and R² comprises 1 or 2 (e.g., 1) oxygen atoms.

In some embodiments, R¹ and R² are different, and R² comprises a sulfur atom.

In some embodiments, R² and R¹ are different, and R² comprises a carbonyl group.

In some embodiments, R² and R¹ are different, and R² comprises 1 or 2 (e.g., 1) nitrogen atoms.

In some embodiments, R² and R¹ are different, and R² comprises 1 or 2 (e.g., 1) oxygen atoms.

In some embodiments, R² and R¹ are different, and R² comprises a sulfur atom.

In some embodiments, R¹ and R² are the same.

In some embodiments, R¹ is para or meta to R².

In some embodiments, R¹ is para or ortho to R².

In some embodiments, R¹ is ortho or meta to R².In some embodiments, R¹ is para to R².

In some embodiments, R¹ is meta to R².

In some embodiments, R¹ is ortho to R² .

In some embodiments of any of the formulae herein, each of R¹ and R² is independently selected from the group consisting of C₁-C₆ alkyl optionally substituted with one or more hydroxy, halo, oxo, or C₁-C₆ alkoxy; C₃-C₇ cycloalkyl optionally substituted with one or more hydroxy, halo, oxo, C₁-C₆ alkoxy, or C₁-C₆ alkyl; wherein the C₁-C₆ alkoxy or C₁-C₆ alkyl is further optionally substituted with one to three hydroxy, halo, NR⁸R⁹, or oxo; 3- to 7-membered heterocycloalkyl optionally substituted with one or more hydroxy, halo, oxo, or C₁-C₆ alkyl wherein the C₁-C₆ alkoxy or C₁-C₆ alkyl is further optionally substituted with one to three hydroxy, halo, or oxo; C₁-C₆ haloalkyl; C₁-C₆ alkoxy; C₁-C₆ haloalkoxy; halo; CN; CO-C₁-C₆ alkyl; CO-C₆-C₁₀ aryl; CO(5- to 10-membered heteroaryl); CO₂C₁-C₆ alkyl; CO₂C₃-C₈ cycloalkyl; OCOC₁-C₆ alkyl; OCOC₆-C₁₀ aryl; OCO(5- to 10-membered heteroaryl); OCO(3- to 7-membered heterocycloalkyl); C₆-C₁₀ aryl; 5- to 10-membered heteroaryl; NH₂; NHC₁-C₆ alkyl; N(C₁-C₆ alkyl)₂; CONR⁸R⁹; SF₅; S(O₂)NR¹¹R¹²; S(O)C₁-C₆ alkyl; and S(O₂)C₁-C₆ alkyl.

In some embodiments of any of the formulae herein, R¹ is selected from the group consisting of 1-hydroxy-2-methylpropan-2-yl; methyl; isopropyl; 2-hydroxy-2-propyl; hydroxymethyl; 1-hydroxyethyl; 2-hydroxyethyl; 1-hydroxy-2-propyl; 1-hydroxy-1-cyclopropyl; 1-hydroxy-1-cyclobutyl; 1-hydroxy-1-cyclopentyl; 1-hydroxy-1-cyclohexyl; morpholinyl; 1,3-dioxolan-2-yl; COCH₃; COCH₂CH₃; 2-methoxy-2-propyl; fluoro; chloro; phenyl; pyridyl; pyrazolyl; S(O₂)CH₃, and S(O₂)NR¹¹R¹².

In some embodiments, R² is selected from the group consisting of fluoro, chloro, cyano, methyl; methoxy; ethoxy; isopropyl; 1-hydroxy-2-methylpropan-2-yl; 2-hydroxy-2-propyl; hydroxymethyl; 1-hydroxyethyl; 2-hydroxyethyl; 1-hydroxy-2-propyl; 1-hydroxy-1-cyclopropyl; COCH₃; COPh; 2-methoxy-2-propyl; S(O₂)CH₃, and S(O₂)NR¹¹R¹².

In some embodiments, one pair of R¹ and R² is on adjacent atoms, and taken together with the atoms connecting them, independently form at least one bicyclic spirocyclic 5- to-12-membered heterocyclic ring containing 1-3 heteroatoms independently selected from O, N, and S, wherein the carbocyclic or heterocyclic ring is optionally substituted with one or more substituents each independently selected from hydroxy, halo, oxo, methyl, isopropoxyl, azetidinyl, oxetanyl, wherein the methyl, isopropoxyl, azetidinyl, and oxetanyl are optionally substituted with one or more substituents each independently selected from hydroxy, fluoro, amino, methylamino, and dimethylamino.

In some embodiments, R¹ and R² are on adjacent atoms, and taken together with the atoms connecting them, independently form one monocyclic or bicyclic C₄-C₁₂ carbocyclic ring or one monocyclic or bicyclic 5- to-12-membered heterocyclic ring containing 1-3 heteroatoms independently selected from O, N, and S, wherein the carbocyclic ring or heterocyclic ring is optionally independently substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR⁸R⁹, ═NR¹⁰, CN, COOC₁-C₆ alkyl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR⁸R⁹, wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR⁸R⁹, ═NR¹⁰, COOC₁-C₆ alkyl, C₆-C₁₀ aryl, and CONR⁸R⁹.

In some embodiments, the optionally substituted ring A is a pyrazolyl; m is 1; n is 1; R¹ and R² are on adjacent atoms, and taken together with the atoms connecting them, independently form one monocyclic or bicyclic C₄-C₁₂ carbocyclic ring or one monocyclic or bicyclic 5- to-12-membered heterocyclic ring containing 1-3 heteroatoms independently selected from O, N, and S, wherein the carbocyclic ring or heterocyclic ring is optionally independently substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR⁸R⁹, ═NR¹⁰, CN, COOC₁-C₆ alkyl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR⁸R⁹, wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR⁸R⁹, ═NR¹⁰, COOC₁-C₆ alkyl, C₆-C₁₀ aryl, and CONR⁸R⁹.

In some embodiments, the optionally substituted ring A is an imidazolyl; m is 1; n is 1; R¹ and R² are on adjacent atoms, and taken together with the atoms connecting them, independently form one monocyclic or bicyclic C₄-C₁₂ carbocyclic ring or one monocyclic or bicyclic 5- to-12-membered heterocyclic ring containing 1-3 heteroatoms independently selected from O, N, and S, wherein the carbocyclic ring or heterocyclic ring is optionally independently substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR⁸R⁹, ═NR¹⁰, CN, COOC₁-C₆ alkyl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR⁸R⁹, wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR⁸R⁹, ═NR¹⁰, COOC₁-C₆ alkyl, C₆-C₁₀ aryl, and CONR⁸R⁹.

In some embodiments, the optionally substituted ring A is a thiophenyl; m is 1; n is 1; R¹ and R² are on adjacent atoms, and taken together with the atoms connecting them, independently form one monocyclic or bicyclic C₄-C₁₂ carbocyclic ring or one monocyclic or bicyclic 5- to-12-membered heterocyclic ring containing 1-3 heteroatoms independently selected from O, N, and S, wherein the carbocyclic ring or heterocyclic ring is optionally independently substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR⁸R⁹, ═NR¹⁰, CN, COOC₁-C₆ alkyl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR⁸R⁹, wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR⁸R⁹, ═NR¹⁰, COOC₁-C₆ alkyl, C₆-C₁₀ aryl, and CONR⁸R⁹.

In some embodiments, the optionally substituted ring A is a thiazolyl; m is 1; n is 1; R¹ and R² are on adjacent atoms, and taken together with the atoms connecting them, independently form one monocyclic or bicyclic C₄-C₁₂ carbocyclic ring or one monocyclic or bicyclic 5- to-12-membered heterocyclic ring containing 1-3 heteroatoms independently selected from O, N, and S, wherein the carbocyclic ring or heterocyclic ring is optionally independently substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR⁸R⁹, ═NR¹⁰, CN, COOC₁-C₆ alkyl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR⁸R⁹, wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR⁸R⁹, ═NR¹⁰, COOC₁-C₆ alkyl, C₆-C₁₀ aryl, and CONR⁸R⁹.

In some embodiments, the optionally substituted ring A is

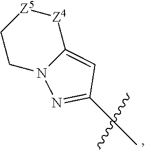

wherein Z⁴ is selected from the group consisting of —CH₂—, —C(O)—, and NH; Z⁵ is selected from the group consisting of O, NH, N—CH₃, and —CH₂—.

The

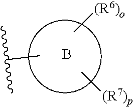

moiety

In some embodiments, the

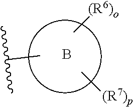

is as defined in (BB-1):

o=1 or 2;

p=0, 1, 2, or 3; and

B is a 6-10 membered monocyclic or bicyclic heteroaryl; wherein at least one R⁶ is ortho to the bond connecting the B ring to the NH(CO) group of Formula AA.

In some embodiments, the

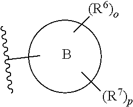

is as defined in (BB-2):

o=1 or 2;

p=0, 1, 2, or 3,

provided that o+p>2;

B is a C₆-C₁₀ monocyclic or bicyclic aryl; wherein at least one R⁶ is ortho to the bond connecting the B ring to the -L-NH(CO) group of Formula AA;

when (BB-1) or (BB-2) applies:

R⁶ and R⁷ are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, C₃-C₁₀ cycloalkyl, 3- to 10-membered heterocycloalkyl, and C₂-C₆ alkenyl,

wherein R⁶ and R⁷ are each optionally substituted with one or more substituents independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), C₆-C₁₀ aryloxy, and S(O₂)C₁-C₆ alkyl; and wherein the C₁-C₆ alkyl or C₁-C₆ alkoxy that R⁶ or R⁷ is substituted with is optionally substituted with one or more hydroxyl, C₆-C₁₀ aryl or NR⁸R⁹, or wherein R⁶ or R⁷ is optionally fused to a five- to -seven-membered carbocyclic ring or heterocyclic ring containing one or two heteroatoms independently selected from oxygen, sulfur and nitrogen;

wherein the 3- to 7-membered heterocycloalkyl, C₆-C₁₀ aryl, 5- to 10-membered heteroaryl are optionally substituted with one or more substituents independently selected from halo, C₁-C₆ alkyl, and OC₁-C₆ alkyl;

or at least one pair of R⁶ and R⁷ on adjacent atoms, taken together with the atoms connecting them, independently form at least one C₄-C₈ carbocyclic ring or at least one 5- to 8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹; OR

provided that:

when (BB-2) applies; o+p=4; two pairs of R⁶ and R⁷ are on adjacent atoms; and

each pair taken together with the atoms connecting them, independently forms a ring selected from a C₄-C₈ carbocyclic ring and a 5- to 8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹,

then at least one ring is selected from:

(a) a C₄ carbocyclic ring, a C₆-C₈ carbocyclic ring, or a 5- to 8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹; and

(b) a C₅ carbocyclic ring substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹.

The Variables o and p

In some embodiments, o=1 or 2.

In some embodiments, o=1.

In some embodiments, o=2.

In some embodiments, p=0, 1, 2, or 3.

In some embodiments, p=0.

In some embodiments, p=1.

In some embodiments, p=2.

In some embodiments, o=1 and p=0.

In some embodiments, o=2 and p=0.

In some embodiments, o=1 and p=1.

In some embodiments, o=1 and p=2.

In some embodiments, o=2 and p=1.

In some embodiments, o=2 and p=2.

In some embodiments, o=2 and p=3.

The Ring B and Substitutions on the Ring B

In some embodiments, B is a 5- to 10-membered monocyclic or bicyclic heteroaryl or a C₆-C₁₀ monocyclic or bicyclic aryl, such as phenyl.

In some embodiments, B is a 5- to 6-membered monocyclic heteroaryl or a C₆ monocyclic aryl.

In some embodiments, B is a 5- to 10-membered monocyclic or bicyclic heteroaryl.

In some embodiments, B is a C₆-C₁₀ monocyclic or bicyclic aryl.

In some embodiments, B is a 5-membered heteroaryl.

In some embodiments, B is a 7-10 membered monocyclic or bicyclic heteroaryl.

In some embodiments, B is phenyl substituted with 1 or 2 R⁶ and optionally substituted with 1, 2, or 3 R⁷.

In some embodiments, B is pyridyl substituted with 1 or 2 R⁶ and optionally substituted with 1, 2, or 3 R⁷.

In some embodiments, B is indazolyl substituted with 1 or 2 R⁶ and optionally substituted with 1, 2, or 3 R⁷.

In some embodiments, B is pyrazolyl substituted with 1 or 2 R⁶ and optionally substituted with 1 or 2 R⁷.

In some embodiments, B is phenyl, o is 1 or 2, and p is 0, 1, 2, or 3.

In some embodiments, B is phenyl, o is 1, and p is 0, 1, 2, or 3.

In some embodiments, B is phenyl, o is 2, and p is 0, 1, 2, or 3.

In some embodiments, B is one of the rings disclosed hereinbelow, substituted as disclosed hereinbelow, wherein in each case the bond that is shown as being broken by the wavy line [symbol] connects B to the NH(CO)group of Formula AA.

In some embodiments, the substituted ring B

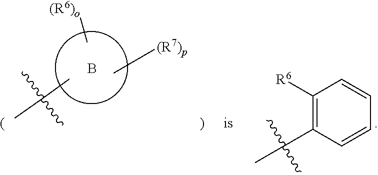

In some embodiments, the substituted ring B

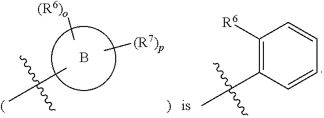

In some embodiments, the substituted ring B is

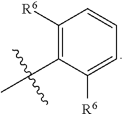

In some embodiments, the substituted ring B is

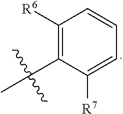

In some embodiments, the substituted ring B is

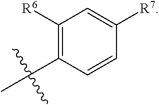

In some embodiments, the substituted ring B is

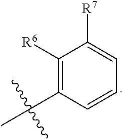

In some embodiments, the substituted ring B is

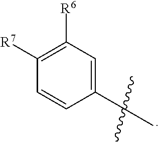

In some embodiments, the substituted ring B is

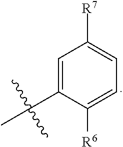

In some embodiments, the substituted ring B is

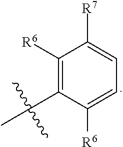

In some embodiments, the substituted ring B is

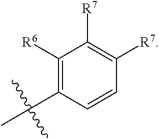

In some embodiments, the substituted ring B is

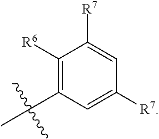

In some embodiments, the substituted ring B is

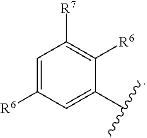

In some embodiments, the substituted ring B is

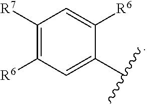

In some embodiments, the substituted ring B is

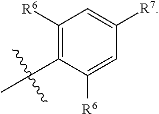

In some embodiments, the substituted ring B is

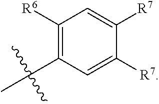

In some embodiments, the substituted ring B is

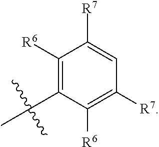

In some embodiments, the substituted ring B is

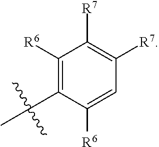

In some embodiments, the substituted ring B is

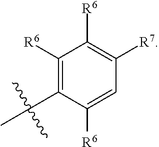

In some embodiments, the substituted ring B is

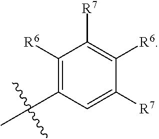

In some embodiments, the substituted ring B is

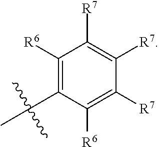

In some embodiments, the substituted ring B is

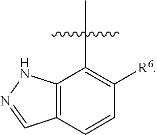

In some embodiments, the substituted ring B is

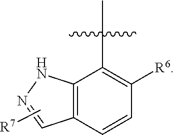

In some embodiments, the substituted ring B is

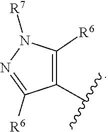

In some embodiments, the substituted ring B is

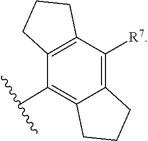

In some embodiments, the substituted ring B is

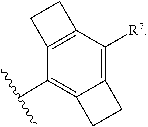

In some embodiments, the substituted ring B is

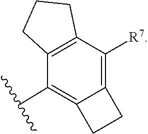

In some embodiments, the substituted ring B is

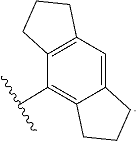 [SMILES: *c1c2c(cc3c1CCC3)CCC2]

In some embodiments, the substituted ring B is

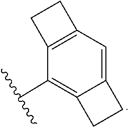 [SMILES: *c1c2c(cc3c1CC3)CC2]

In some embodiments, the substituted ring B is

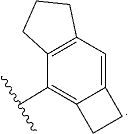 [SMILES: *c1c2c(cc3c1CC3)CCC2]

In some embodiments, the substituted ring B is

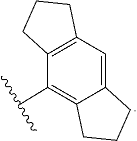 [SMILES: *c1c2c(cc3c1CCC3)CCC2]

In some embodiments, the substituted ring B is

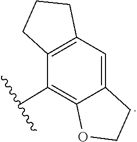 [SMILES: *c1c2c(cc3c1OCC3)CCC2]

In some embodiments, the substituted ring B is

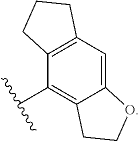 [SMILES: *c1c2c(cc3c1CCO3)CCC2]

In some embodiments, the substituted ring B is

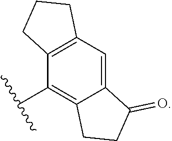 [SMILES: *c1c2c(cc3c1CCC3=O)CCC2]

In some embodiments, the substituted ring B is

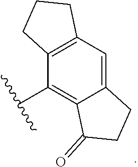 [SMILES: *c1c2c(cc3c1C(=O)CC3)CCC2]

In some embodiments, the substituted ring B is

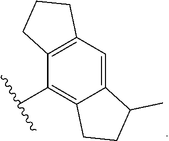 [SMILES: *c1c2c(cc3c1CCC3C)CCC2]

In some embodiments, the substituted ring B is

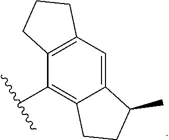 [SMILES: *c1c2c(cc3c1CC[C@@H]3C)CCC2]

In some embodiments, the substituted ring B is

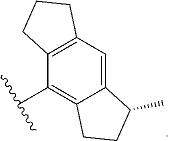 [SMILES: *c1c2c(cc3c1CC[C@H]3C)CCC2]

In some embodiments, the substituted ring B is

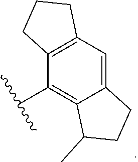 [SMILES: *c1c2c(cc3c1C(C)CC3)CCC2]

In some embodiments, the substituted ring B is

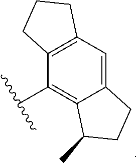 [SMILES: *c1c2c(cc3c1[C@H](C)CC3)CCC2]

In some embodiments, the substituted ring B is

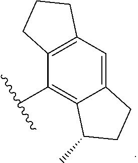 [SMILES: *c1c2c(cc3c1[C@@H](C)CC3)CCC2]

In some embodiments, the substituted ring B is

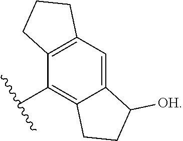 [SMILES: *c1c2c(cc3c1CCC3O)CCC2]

In some embodiments, the substituted ring B is

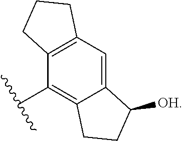 [SMILES: *c1c2c(cc3c1CC[C@@H]3O)CCC2]

In some embodiments, the substituted ring B is

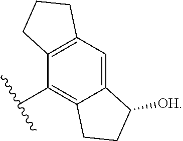 [SMILES: *c1c2c(cc3c1CC[C@H]3O)CCC2]

In some embodiments, the substituted ring B is

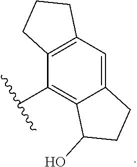 [SMILES: *c1c2c(cc3c1C(O)CC3)CCC2]

In some embodiments, the substituted ring B is

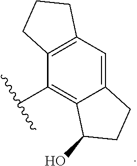 [SMILES: *c1c2c(cc3c1[C@H](O)CC3)CCC2]

In some embodiments, the substituted ring B is

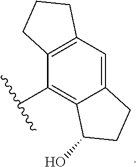 [SMILES: *c1c2c(cc3c1[C@@H](O)CC3)CCC2]

In some embodiments, the substituted ring B is

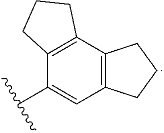 [SMILES: *c1cc2c(c3c1CCC3)CCC2]

In some embodiments, the substituted ring B is

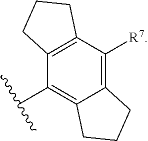

In some embodiments, B is pyridyl or an N-oxide thereof (e.g., 2-pyridyl or an N-oxide thereof, 3-pyridyl or an N-oxide thereof, or 4-pyridyl or an N-oxide thereof).

In some embodiments, B is pyridyl (e.g., 2-pyridyl, 3-pyridyl, or 4-pyridyl).

In some embodiments, B is a pyridyl N-oxide (e.g., 2-pyridyl N-oxide, 3-pyridyl N-oxide, or 4-pyridyl N-oxide).

In some embodiments, B is pyrimidinyl or an N-oxide thereof (e.g., 4-pyrimidinyl or an N-oxide thereof, or 5-pyrimidinyl or an N-oxide thereof).

In some embodiments, B is pyridazinyl.

In some embodiments, B is pyrazinyl.

In some embodiments, B is triazinyl.

In some embodiments, B is one of the rings disclosed hereinbelow, substituted as disclosed hereinbelow, wherein in each case the bond that is shown as being broken by the wavy line [symbol] connects B to the NHC(O)group of Formula AA.

In some embodiments, the substituted ring B is

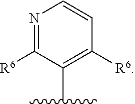

In some embodiments, the substituted ring B is

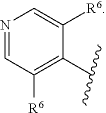

In some embodiments, the substituted ring B is

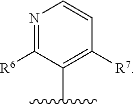

In some embodiments, the substituted ring B is

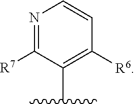

In some embodiments, the substituted ring B is

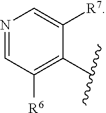

In some embodiments, the substituted ring B is

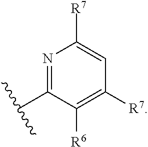

In some embodiments, the substituted ring B is

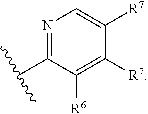

In some embodiments, the substituted ring B is

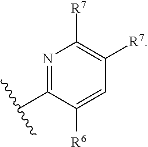

In some embodiments, the substituted ring B is

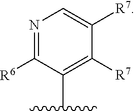

In some embodiments, the substituted ring B is

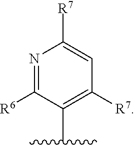

In some embodiments, the substituted ring B is

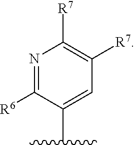

In some embodiments, the substituted ring B is

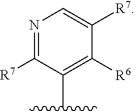

In some embodiments, the substituted ring B is

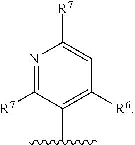

In some embodiments, the substituted ring B is

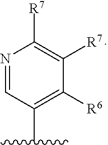

In some embodiments, the substituted ring B is

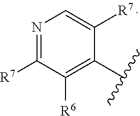

In some embodiments, the substituted ring B is

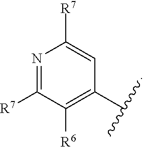

In some embodiments, the substituted ring B is

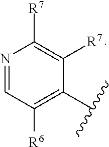

In some embodiments, the substituted ring B is

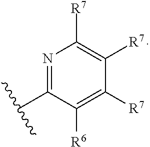

In some embodiments, the substituted ring B is

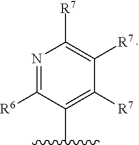

In some embodiments, the substituted ring B is

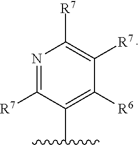

In some embodiments, the substituted ring B is

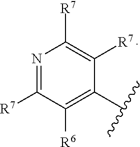

In some embodiments, the substituted ring B is

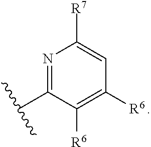

In some embodiments, the substituted ring B is

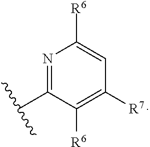

In some embodiments, the substituted ring B is

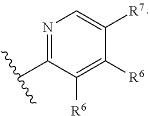

In some embodiments, the substituted ring B is

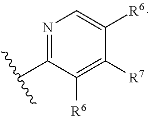

In some embodiments, the substituted ring B is

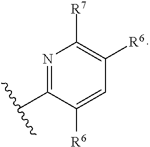

In some embodiments, the substituted ring B is

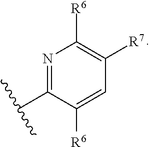

In some embodiments, the substituted ring B is

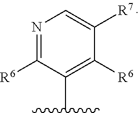

In some embodiments, the substituted ring B is

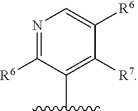

In some embodiments, the substituted ring B is

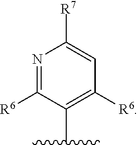

In some embodiments, the substituted ring B is

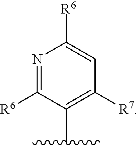

In some embodiments, the substituted ring B is

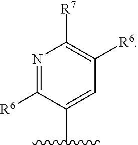

In some embodiments, the substituted ring B is

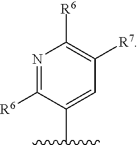

In some embodiments, the substituted ring B is

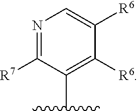

In some embodiments, the substituted ring B is

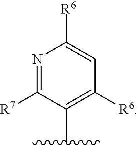

In some embodiments, the substituted ring B is

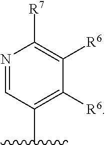

In some embodiments, the substituted ring B is

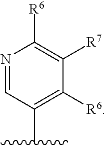

In some embodiments, the substituted ring B is

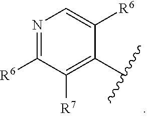

In some embodiments, the substituted ring B is

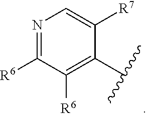

In some embodiments, the substituted ring B is

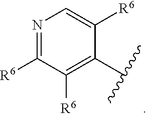

In some embodiments, the substituted ring B is

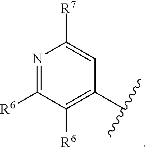

In some embodiments, the substituted ring B is

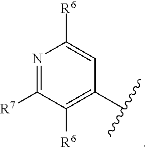

In some embodiments, the substituted ring B is

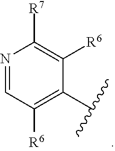

In some embodiments, the substituted ring B is

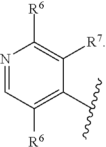

In some embodiments, the substituted ring B is

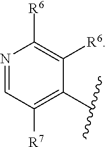

In some embodiments, the substituted ring B is

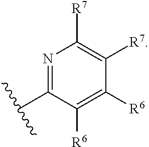

In some embodiments, the substituted ring B is

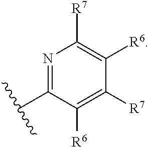

In some embodiments, the substituted ring B is

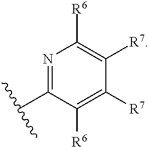

In some embodiments, the substituted ring B is

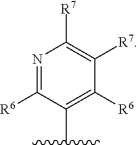

In some embodiments, the substituted ring B is

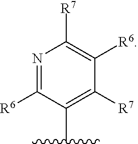

In some embodiments, the substituted ring B is

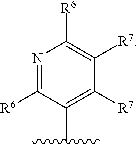

In some embodiments, the substituted ring B is

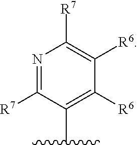

In some embodiments, the substituted ring B is

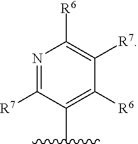

In some embodiments, the substituted ring B is

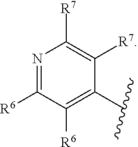

In some embodiments, the substituted ring B is

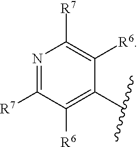

In some embodiments, the substituted ring B is

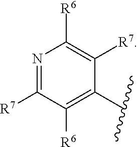

In some embodiments, the substituted ring B is

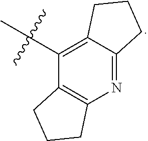 [SMILES: *c1c2c(nc3c1CCC3)CCC2]

In some embodiments, the substituted ring B is

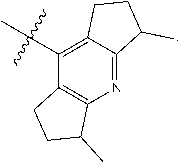 [SMILES: *c1c2c(nc3c1CCC3C)C(C)CC2]

In some embodiments, the substituted ring B is

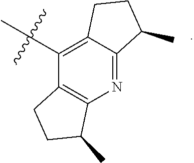 [SMILES: *c1c2c(nc3c1CC[C@@H]3C)[C@H](C)CC2]

In some embodiments, the substituted ring B is

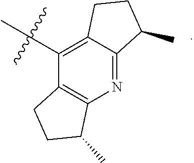 [SMILES: *c1c2c(nc3c1CC[C@H]3C)[C@H](C)CC2]

In some embodiments, the substituted ring B is

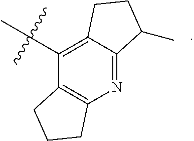 [SMILES: *c1c2c(nc3c1CCC3C)CCC2]

In some embodiments, the substituted ring B is

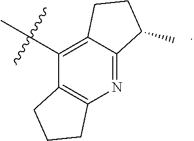 [SMILES: *c1c2c(nc3c1CC[C@@H]3C)CCC2]

In some embodiments, the substituted ring B is

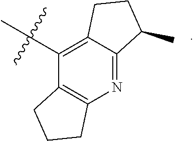 [SMILES: *c1c2c(nc3c1CC[C@H]3C)CCC2]

In some embodiments, the substituted ring B is

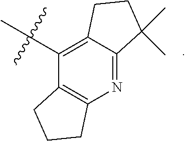 [SMILES: *c1c2c(nc3c1CCC3(C)C)CCC2]

In some embodiments, the substituted ring B is

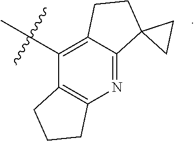 [SMILES: *c1c2c(nc3c1CCC31CC1)CCC2]

In some embodiments, the substituted ring B is

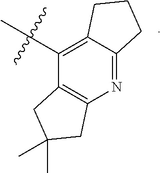 [SMILES: *c1c2c(nc3c1CC(C)(C)C3)CCC2]

In some embodiments, the substituted ring B is

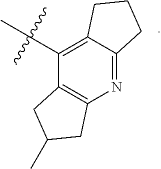 [SMILES: *c1c2c(nc3c1CC(C)C3)CCC2]

In some embodiments, the substituted ring B is

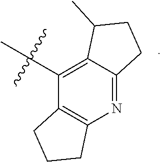 [SMILES: *c1c2c(nc3c1C(C)CC3)CCC2]

In some embodiments, the substituted ring B is

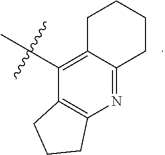 [SMILES: *c1c2c(nc3c1CCC3)CCCC2]

In some embodiments, the substituted ring B is

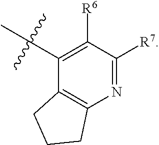

In some embodiments, the substituted ring B is

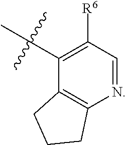

In some embodiments, the substituted ring B is

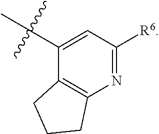

In some embodiments, the substituted ring B is

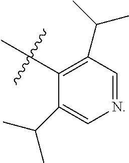 [SMILES: *c1c(C(C)C)cncc1C(C)C]

In some embodiments, the substituted ring B is

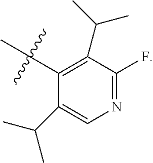 [SMILES: *c1c(C(C)C)cnc(F)c1C(C)C]

In some embodiments, the substituted ring B is

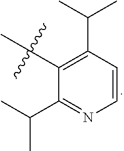 [SMILES: *c1c(C(C)C)ccnc1C(C)C]

In some embodiments, the substituted ring B is

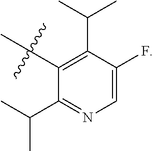 [SMILES: *c1c(C(C)C)ncc(F)c1C(C)C]

In some embodiments, the substituted ring B is

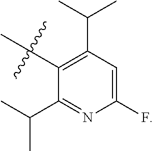 [SMILES: *c1c(C(C)C)cc(F)nc1C(C)C]

In some embodiments, the substituted ring B is selected from the group consisting of:

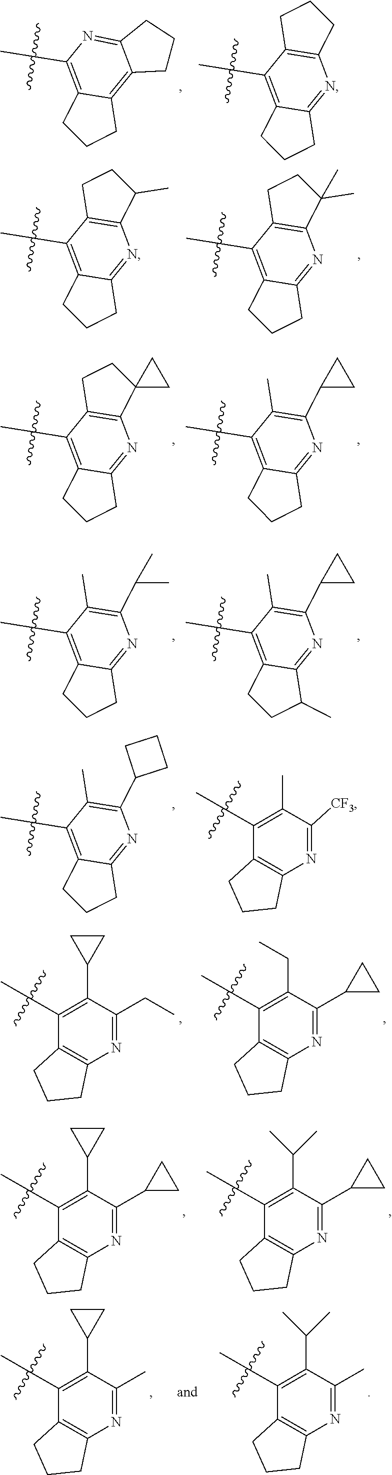

The Groups R⁶ and R⁷

In some embodiments,

R⁶ and R⁷ are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, S(O₂)C₁-C₆ alkyl, C₃-C₁₀ cycloalkyl and 3- to 10-membered heterocycloalkyl, and a C₂-C₆ alkenyl,

wherein R⁶ and R⁷ are each optionally substituted with one or more substituents independently selected from

hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), NHCOC₂-C₆ alkynyl,

C₆-C₁₀ aryloxy, and S(O₂)C₁-C₆ alkyl; and wherein the C₁-C₆ alkyl or C₁-C₆ alkoxy that R⁶ or R⁷ is substituted with is optionally substituted with one or more hydroxyl, C₆-C₁₀ aryl or NR⁸R⁹, or wherein R⁶ or R⁷ is optionally fused to a five- to -seven-membered carbocyclic ring or heterocyclic ring containing one or two heteroatoms independently selected from oxygen, sulfur and nitrogen;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;

or at least one pair of R⁶ and R⁷ on adjacent atoms, taken together with the atoms connecting them, independently form at least one C₄-C₈ carbocyclic ring or at least one 5-to 8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹.

In some embodiments,

R⁶ and R⁷ are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, S(O₂)C₁-C₆ alkyl, C₃-C₁₀ cycloalkyl and 3- to 10-membered heterocycloalkyl, and a C₂-C₆ alkenyl,

wherein R⁶ and R⁷ are each optionally substituted with one or more substituents independently selected from

hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), NHCOC₂-C₆ alkynyl,

C₆-C₁₀ aryloxy, and S(O₂)C₁-C₆ alkyl; and wherein the C₁-C₆ alkyl or C₁-C₆ alkoxy that R⁶ or R⁷ is substituted with is optionally substituted with one or more hydroxyl, C₆-C₁₀ aryl or NR⁸R⁹, or wherein R⁶ or R⁷ is optionally fused to a five- to -seven-membered carbocyclic ring or heterocyclic ring containing one or two heteroatoms independently selected from oxygen, sulfur and nitrogen;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;

or at least one pair of R⁶ and R⁷ on adjacent atoms, taken together with the atoms connecting them, independently form at least one C₄-C₆ aliphatic carbocyclic ring or at least one 5-to 6-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹.

In some embodiments,

R⁶ and R⁷ are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, C₃-C₇ cycloalkyl and 3- to 7-membered heterocycloalkyl,

wherein the C₁-C₆ alkyl, C₁-C₆ haloalkyl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;

or at least one pair of R⁶ and R⁷ on adjacent atoms, taken together with the atoms connecting them, independently form at least one C₄-C₈ carbocyclic ring or at least one 5- to 8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹.

In some embodiments,

R⁶ and R⁷ are each independently selected from C₁-C₆ haloalkyl, C₁-C₆ alkoxy, C₁-C₆ haloalkoxy, halo, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, C₃-C₇ cycloalkyl and 3- to 7-membered heterocycloalkyl, wherein the C₃-C₇ cycloalkyl, C₁-C₆ haloalkyl, and 3- to 7-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;

or at least one pair of R⁶ and R⁷ on adjacent atoms, taken together with the atoms connecting them, independently form at least one C₄-C₈ carbocyclic ring or at least one 5- to 8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹.

In some embodiments,

R⁶ and R⁷ are each independently selected from C₁-C₆ alkyl, halo, CN, NO₂, COC₁-C₆ alkyl, CO₂C₁-C₆ alkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, C₃-C₇ cycloalkyl and 3- to 7-membered heterocycloalkyl,

wherein the C₁-C₆ alkyl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;

or at least one pair of R⁶ and R⁷ on adjacent atoms, taken together with the atoms connecting them, independently form at least one C₄-C₈ carbocyclic ring or at least one 5- to 8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹.

In some embodiments,

R⁶ and R⁷ are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, C₃-C₇ cycloalkyl and 3- to 7-membered heterocycloalkyl,

wherein the C₁-C₆ alkyl, C₁-C₆ haloalkyl, C₃-C₇ cycloalkyl, and 3- to 7-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are unsubstituted;

or at least one pair of R⁶ and R⁷ on adjacent atoms, taken together with the atoms connecting them, independently form at least one C₄-C₈ carbocyclic ring or at least one 5- to 8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹.

In some embodiments,

R⁶ and R⁷ are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, C₃-C₇ cycloalkyl and 3- to 7-membered heterocycloalkyl,

wherein the C₁-C₆ alkyl, C₃-C₇ cycloalkyl and 3- to 7-membered heterocycloalkyl are each unsubstituted;

or at least one pair of R⁶ and R⁷ on adjacent atoms, taken together with the atoms connecting them, independently form at least one C₄-C₈ carbocyclic ring or at least one 5- to 8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹.

In some embodiments,

R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO-C₁-C₆ alkyl; CONR⁸R⁹, and 4- to 6-membered heterocycloalkyl,

wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;
and R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy;

or R⁶ and R⁷, taken together with the atoms connecting them, independently form C₄-C₇ carbocyclic ring or at least one 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments,

R⁶ and R⁷ are each independently selected from C₁-C₆ alkyl, C₁-C₆ alkoxy, halo, CN, NO₂, COC₁-C₆ alkyl, CO₂C₁-C₆ alkyl, C₆-C₁₀ aryl, 5- to 10-membered heteroaryl, CONR⁸R⁹, and 3- to 7-membered heterocycloalkyl,

wherein the C₁-C₆ alkyl and 3- to 7-membered heterocycloalkyl is optionally substituted with one or more substituents each independently selected from hydroxy or oxo,

or at least one pair of R⁶ and R⁷ on adjacent atoms, taken together with the atoms connecting them, independently form at least one C₄-C₈ carbocyclic ring, wherein the carbocyclic ring is optionally independently substituted with one or more hydroxy or oxo.

In some embodiments,

R⁶ and R⁷ are each independently selected from C₁-C₆ alkyl, C₁-C₆ alkoxy, halo, CN, NO₂, COC₁-C₆ alkyl, CO₂C₁-C₆ alkyl, C₆-C₁₀ aryl, 5- to 10-membered heteroaryl, CONR⁸R⁹, and 3- to 7-membered heterocycloalkyl,

wherein the C₁-C₆ alkyl and 3- to 7-membered heterocycloalkyl is optionally substituted with one or more substituents each independently selected from hydroxy or oxo,

or at least one pair of R⁶ and R⁷ on adjacent atoms, taken together with the atoms connecting them, independently form at least one C₄-C₆ aliphatic carbocyclic ring, wherein the carbocyclic ring is optionally independently substituted with one or more hydroxy or oxo.

In some embodiments,

R⁶ and R⁷ are each independently selected from C₁-C₆ alkyl, C₁-C₆ alkoxy, halo, CN, NO₂, COC₁-C₆ alkyl, CO₂C₁-C₆ alkyl, C₆-C₁₀ aryl, 5- to 10-membered heteroaryl, CONR⁸R⁹, and 3- to 7-membered heterocycloalkyl,

wherein the C₁-C₆ alkyl and 3- to 7-membered heterocycloalkyl is optionally substituted with one or more substituents each independently selected from hydroxy or oxo,

or at least one pair of R⁶ and R⁷ on adjacent atoms, taken together with the atoms connecting them, independently form at least one 5-to 8-membered heterocyclic ring containing 1 or 2 heteroatoms independently selected from O, N, and S, wherein the heterocyclic ring is optionally independently substituted with one or more hydroxy or oxo.

In some embodiments,

R⁶ and R⁷ are each independently selected from C₁-C₆ alkyl, C₁-C₆ alkoxy, halo, CN, NO₂, COC₁-C₆ alkyl, CO₂C₁-C₆ alkyl, C₆-C₁₀ aryl, 5- to 10-membered heteroaryl, CONR⁸R⁹, and 3- to 7-membered heterocycloalkyl,

wherein the C₁-C₆ alkyl and 3- to 7-membered heterocycloalkyl is optionally substituted with one or more substituents each independently selected from hydroxy or oxo,

or at least one pair of R⁶ and R⁷ on adjacent atoms, taken together with the atoms connecting them, independently form at least one C₄-C₈ carbocyclic ring, wherein the carbocyclic ring is optionally independently substituted with one or more hydroxy or oxo.

In some embodiments,

at least one pair of R⁶ and R⁷ on adjacent atoms, taken together with the atoms connecting them, independently form at least one C₄-C₆ aliphatic carbocyclic ring, wherein the carbocyclic ring is optionally independently substituted with one or more hydroxy or oxo.

In some embodiments,

at least one pair of R⁶ and R⁷ on adjacent atoms, taken together with the atoms connecting them, independently form at least one C₄ aliphatic carbocyclic ring, wherein the carbocyclic ring is optionally independently substituted with one or more hydroxy or oxo.

In some embodiments,

at least one pair of R⁶ and R⁷ on adjacent atoms, taken together with the atoms connecting them, independently form at least one C₅ aliphatic carbocyclic ring, wherein the carbocyclic ring is optionally independently substituted with one or more hydroxy or oxo.

In some embodiments,

at least one pair of R⁶ and R⁷ on adjacent atoms, taken together with the atoms connecting them, independently form at least one C₆ aliphatic carbocyclic ring, wherein the carbocyclic ring is optionally independently substituted with one or more hydroxy or oxo.

In some embodiments,

at least one pair of R⁶ and R⁷ on adjacent atoms, taken together with the atoms connecting them, independently form at least one 5-to 6-membered heterocyclic ring containing 1 heteroatom independently selected from O, N, and S, wherein the heterocyclic ring is optionally independently substituted with one or more hydroxy or oxo.

In some embodiments,

at least one pair of R⁶ and R⁷ on adjacent atoms, taken together with the atoms connecting them, independently form at least one 5-membered heterocyclic ring containing 1 heteroatom independently selected from O, N, and S, wherein the heterocyclic ring is optionally independently substituted with one or more hydroxy or oxo.

In some embodiments,

at least one pair of R⁶ and R⁷ on adjacent atoms, taken together with the atoms connecting them, independently form at least one 6-membered heterocyclic ring containing 1 heteroatom independently selected from O, N, and S, wherein the heterocyclic ring is optionally independently substituted with one or more hydroxy or oxo.

In some embodiments,

at least one pair of R⁶ and R⁷ on adjacent atoms, taken together with the atoms connecting them, independently form at least one C₄ aliphatic carbocyclic ring, wherein the carbocyclic ring is optionally independently substituted with one or more C₁-C₆ alkyl.

In some embodiments,

at least one pair of R⁶ and R⁷ on adjacent atoms, taken together with the atoms connecting them, independently form at least one C₅ aliphatic carbocyclic ring, wherein the carbocyclic ring is optionally independently substituted with one or more C₁-C₆ alkyl.

In some embodiments,

at least one pair of R⁶ and R⁷ on adjacent atoms, taken together with the atoms connecting them, independently form at least one C₆ aliphatic carbocyclic ring, wherein the carbocyclic ring is optionally independently substituted with one or more C₁-C₆ alkyl.

In some embodiments,

at least one pair of R⁶ and R⁷ on adjacent atoms, taken together with the atoms connecting them, independently form at least one 5-to 6-membered heterocyclic ring containing 1 heteroatom independently selected from O, N, and S, wherein the heterocyclic ring is optionally independently substituted with one or more C₁-C₆ alkyl.

In some embodiments,

at least one pair of R⁶ and R⁷ on adjacent atoms, taken together with the atoms connecting them, independently form at least one 5-membered heterocyclic ring containing 1 heteroatom independently selected from O, N, and S, wherein the heterocyclic ring is optionally independently substituted with one or more C₁-C₆ alkyl.

In some embodiments,

at least one pair of R⁶ and R⁷ on adjacent atoms, taken together with the atoms connecting them, independently form at least one 6-membered heterocyclic ring containing 1 heteroatom independently selected from O, N, and S, wherein the heterocyclic ring is optionally independently substituted with one or more C₁-C₆ alkyl.

In some embodiments, o=1; p=0; and

R⁶ is selected from C₁-C₆ alkyl, C₁-C₆ haloalkyl, C₁-C₆ alkoxy, C₁-C₆ haloalkoxy, halo, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, C₃-C₇ cycloalkyl and 3- to 7-membered heterocycloalkyl,

wherein the C₁-C₆ alkyl, C₁-C₆ haloalkyl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl.

In some embodiments, o=1; p=1; and

R⁶ and R⁷ are each independently selected from C₁-C₆ alkyl, C₁-C₆ alkoxy, halo, CN, NO₂, COC₁-C₆ alkyl, CO₂C₁-C₆ alkyl, C₆-C₁₀ aryl, 5- to 10-membered heteroaryl, CONR⁸R⁹, and 3- to 7-membered heterocycloalkyl,

wherein the C₁-C₆ alkyl and 3- to 7-membered heterocycloalkyl is optionally substituted with one or more substituents each independently selected from hydroxy or oxo,

or at least one pair of R⁶ and R⁷ on adjacent atoms, taken together with the atoms connecting them, independently form at least one C₄-C₈ carbocyclic ring, wherein the carbocyclic ring is optionally independently substituted with one or more hydroxy or oxo.

In some embodiments, o=1 or 2; p=1, 2, or 3; and

R⁶ and R⁷ are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, C₃-C₇ cycloalkyl and 3- to 7-membered heterocycloalkyl,

wherein the C₁-C₆ alkyl, C₁-C₆ haloalkyl, C₃-C₇ cycloalkyl, and 3- to 7-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl.

In some embodiments, o=2; p=1; and

each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO-C₁-C₆ alkyl; CONR⁸R⁹, and 4- to 6-membered heterocycloalkyl,

wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;
and R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy;

or R⁶ and R⁷, taken together with the atoms connecting them, independently form C₄-C₇ carbocyclic ring or at least one 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, o=2; p=2 or 3; and

each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO-C₁-C₆ alkyl; CONR⁸R⁹, and 4- to 6-membered heterocycloalkyl,

wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;
wherein each R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy;

or at least one pair of R⁶ and R⁷ on adjacent atoms, taken together with the atoms connecting them, independently form at least one C₄-C₇ (e.g., C₄-C6) carbocyclic ring (e.g., aliphatic carbocyclic ring) or at least one 5-to-7-membered (e.g., 5-to-6-membered) heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹.

In some embodiments, o=1 or 2; p=1, 2, or 3; and

R⁶ and R⁷ are each independently selected from C₁-C₆ alkyl, C₁-C₆ alkoxy, halo, CN, NO₂, COC₁-C₆ alkyl, CO₂C₁-C₆ alkyl, C₆-C₁₀ aryl, 5- to 10-membered heteroaryl, CONR⁸R⁹, and 3- to 7-membered heterocycloalkyl,

wherein the C₁-C₆ alkyl and 3- to 7-membered heterocycloalkyl is optionally substituted with one or more substituents each independently selected from hydroxy or oxo,

or at least one pair of R⁶ and R⁷ on adjacent atoms, taken together with the atoms connecting them, independently form at least one C₄-C₈ carbocyclic ring, wherein the carbocyclic ring is optionally independently substituted with one or more hydroxy or oxo.

In some embodiments, o=1 or 2; p=1, 2, or 3; and

R⁶ and R⁷ are each independently selected from C₁-C₆ alkyl, C₁-C₆ alkoxy, halo, CN, NO₂, COC₁-C₆ alkyl, CO₂C₁-C₆ alkyl, C₆-C₁₀ aryl, 5- to 10-membered heteroaryl, CONR⁸R⁹, and 3- to 7-membered heterocycloalkyl,

wherein the C₁-C₆ alkyl and 3- to 7-membered heterocycloalkyl is optionally substituted with one or more substituents each independently selected from hydroxy or oxo.

In some embodiments, o=1 or 2; p=1, 2, or 3; and

one R⁶ and one R⁷ are on adjacent atoms, and taken together with the atoms connecting them, form a C₄-C₈ carbocyclic ring or a 5- to 8-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, o=1 or 2; p=1, 2, or 3; and

one R⁶ and one Ware on adjacent atoms, and taken together with the atoms connecting them, form a C₆ carbocyclic ring or a 5-to-6-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, o=1 or 2; p=1, 2, or 3; and

one R⁶ and one R⁷ are on adjacent atoms, and taken together with the atoms connecting them, form a C₄-C₈ carbocyclic ring or a 5- to 8-membered heterocyclic ring containing 1 or 2 heteroatoms independently selected from O, N, and S, wherein the carbocyclic ring or heterocyclic ring is unsubstituted.

In some embodiments, o=2; p=2 or 3; and

two pairs, each of one R⁶ and one R⁷, are on adjacent atoms, and each pair of one R⁶ and one R⁷ taken together with the atoms connecting them independently form a C₄-C₈ carbocyclic ring or a 5- to 8-membered heterocyclic ring containing 1 or 2 heteroatoms independently selected from O, N, and S, wherein each carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, o=2; p=2 or 3; and

two pairs, each of one R⁶ and one R⁷, are on adjacent atoms, and each pair of one R⁶ and one R⁷ taken together with the atoms connecting them independently form a C₆ carbocyclic ring or a 5-to-6-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, o=2; p=2 or 3; and

two pairs, each of one R⁶ and one R⁷, are on adjacent atoms, and each pair of one R⁶ and one R⁷ taken together with the atoms connecting them independently form a C₅ carbocyclic ring, wherein the carbocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, o=2; p=2 or 3; and

two pairs, each of one R⁶ and one R⁷, are on adjacent atoms, and each pair of one R⁶ and one R⁷ taken together with the atoms connecting them independently form a C₄ carbocyclic ring, wherein the carbocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, o=2; p=2 or 3; and

two pairs, each of one R⁶ and one R⁷, are on adjacent atoms, one pair of one R⁶ and one R⁷ taken together with the atoms connecting them independently form a C₄ carbocyclic ring, and the other pair of one R⁶ and one R⁷ taken together with the atoms connecting them independently form a C₅ carbocyclic ring, wherein each of C₄ and C₅ carbocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, o=2; p=2 or 3; and

two pairs, each of one R⁶ and one R⁷, are on adjacent atoms, one pair of one R⁶ and one R⁷ taken together with the atoms connecting them independently form a C₅ carbocyclic ring, and the other pair of one R⁶ and one R⁷ taken together with the atoms connecting them independently form a 5- to 8-membered heterocyclic ring containing 1 or 2 heteroatoms independently selected from O, N, and S (e.g., a 5-membered heteorocyclic ring, e.g., 5-membered heterocyclic ring containing 1 heteroatom), wherein each of carbocyclic and heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, o=2; p=2 or 3; and

two pairs, each of one R⁶ and one R⁷, are on adjacent atoms, and each pair of one R⁶ and one R⁷ taken together with the atoms connecting them independently form a C₄-C₈ carbocyclic ring or a 5- to 8-membered heterocyclic ring containing 1 or 2 heteroatoms independently selected from O, N, and S, wherein the carbocyclic ring or heterocyclic ring is unsubstituted.

Particular Embodiments wherein o=1; p=0:

In some embodiments, R⁶ is C₁-C₆ alkyl.

In some embodiments, R⁶ is isopropyl.

In some embodiments, R⁶ is ethyl.

In some embodiments, R⁶ is methyl.

In some embodiments, R⁶ is C₁-C₆ alkyl substituted with one or more halo.

In some embodiments, R⁶ is trifluoromethyl.

In some embodiments, R⁶ is trifluoromethoxy.

In some embodiments, R⁶ is C₃-C₇ cycloalkyl.

In some embodiments, R⁶ is cyclopropyl.

In some embodiments, R⁶ is halo.

In some embodiments, R⁶ is chloro.

In some embodiments, R⁶ is fluoro.

In some embodiments, R⁶ is cyano.

In some embodiments, R⁶ is attached to a carbon of an aryl ring B.

In some embodiments, R⁶ is attached to a carbon of a heteroaryl ring B.

In some embodiments, R⁶ is attached to a nitrogen of a heteroaryl ring B.

Particular Embodiments wherein o=1 or 2; p=1, 2, or 3:

In some embodiments, at least one R⁶ is C₁-C₆ alkyl, and at least one R⁷ is C₁-C₆ alkyl optionally substituted with one or more halo.

In some embodiments, at least one R⁶ is C₁-C₆ alkyl and at least one R⁷ is C₁-C₆ alkyl.

In some embodiments, at least one R⁶ is isopropyl and at least one R⁷ is methyl.

In some embodiments, at least one R⁶ is isopropyl and at least one R⁷ is isopropyl.

In some embodiments, o=1; p=1; R⁶ is isopropyl; and R⁷ is isopropyl.

In some embodiments, at least one R⁶ is C₁-C₆ alkyl, and at least one R⁷ is C₁-C₆ alkyl substituted with one or more halo.

In some embodiments, at least one R⁶ is isopropyl and at least one R⁷ is trifluoromethyl.

In some embodiments, at least one R⁶ is C₁-C₆ alkyl, and at least one R⁷ is C₃-C₇ cycloalkyl.

In some embodiments, at least one R⁶ is isopropyl and at least one R⁷ is cyclopropyl.

In some embodiments, o=1; p=1; R⁶ is isopropyl; and R⁷ is cyclopropyl.

In some embodiments, at least one R⁶ is C₁-C₆ alkyl, and at least one R⁷ is halo.

In some embodiments, at least one R⁶ is isopropyl and at least one R⁷ is halo.

In some embodiments, at least one R⁶ is isopropyl and at least one R⁷ is chloro.

In some embodiments, at least one R⁶ is isopropyl and at least one R⁷ is fluoro.

In some embodiments, o=1; p=1; R⁶ is isopropyl; and R⁷ is chloro.

In some embodiments, o=2; p=1; at least one R⁶ is isopropyl; and R⁷ is chloro.

In some embodiments, o=1; p=1; R⁶ is isopropyl; and R⁷ is fluoro.

In some embodiments, o=2; p=1; at least one R⁶ is isopropyl; and R⁷ is fluoro.

In some embodiments, o=2; p=2; at least one R⁶ is isopropyl; and at least one R⁷ is fluoro.

In some embodiments, o=2; p=2; at least one R⁶ is isopropyl; one R⁷ is fluoro; and the other R⁷ is cyano.

In some embodiments, o=2; p=3; at least one R⁶ is isopropyl; two R⁷ are fluoro; and one R⁷ is chloro.

In some embodiments, o=2; p=1; at least one R⁶ is ethyl; and R⁷ is fluoro.

In some embodiments, o=2; p=1; one R⁶ is isopropyl; the other R⁶ is trifluoromethyl; and R⁷ is chloro.

In some embodiments, at least one R⁶ is C₁-C₆ alkyl, and at least one R⁷ is cyano.

In some embodiments, at least one R⁶ is isopropyl and at least one R⁷ is cyano.

In some embodiments, o=1; p=1; R⁶ is isopropyl; and R⁷ is cyano.

In some embodiments, o=2; p=1; at least one R⁶ is isopropyl; and R⁷ is cyano.

In some embodiments, at least one R⁶ is C₃-C₇ cycloalkyl, and at least one R⁷ is C₃-C₇ cycloalkyl.

In some embodiments, at least one R⁶ is cyclopropyl, and at least one R⁷ is cyclopropyl.

In some embodiments, at least one R⁶ is C₃-C₇ cycloalkyl, and at least one R⁷ is halo.

In some embodiments, at least one R⁶ is cyclopropyl and at least one R⁷ is halo.

In some embodiments, at least one R⁶ is cyclopropyl and at least one R⁷ is chloro.

In some embodiments, at least one R⁶ is cyclopropyl and at least one R⁷ is fluoro.

In some embodiments, o=1; p=1; R⁶ is cyclopropyl; and R⁷ is chloro.

In some embodiments, o=1; p=1; R⁶ is cyclopropyl; and R⁷ is fluoro.

In some embodiments, at least one R⁶ is C₁-C₆ alkyl, and at least one R⁷ is C₁-C₆ alkoxy optionally substituted with one or more halo.

In some embodiments, at least one R⁶ is isopropyl, and at least one R⁷ is C₁-C₆ alkoxy.

In some embodiments, at least one R⁶ is isopropyl, and at least one R⁷ is methoxy.

In some embodiments, o=1; p=1; R⁶ is isopropyl, and R⁷ is methoxy.

In some embodiments, o=2; p=1; at least one R⁶ is isopropyl, and R⁷ is methoxy.

In some embodiments, at least one R⁶ is C₁-C₆ alkyl, and at least one R⁷ is C₁-C₆ alkoxy substituted with one or more halo.

In some embodiments, at least one R⁶ is isopropyl, and at least one R⁷ is trifluoromethoxy.

In some embodiments, at least one R⁶ is isopropyl, and at least one R⁷ is difluoromethoxy.

In some embodiments, at least one R⁶ is halo, and at least one R⁷ is C₁-C₆ haloalkyl optionally substituted with hydroxy.

In some embodiments, o=1; p=1; R⁶ is chloro, and R⁷ is trifluoromethyl.

In some embodiments, at least one R⁶ is halo, and at least one R⁷ is C₁-C₆ haloalkoxy.

In some embodiments, at least one R⁶ is chloro, and at least one R⁷ is trifluoromethoxy.

In some embodiments, o=1; p=1; R⁶ is chloro, and R⁷ is trifluoromethoxy.

In some embodiments, at least one R⁶ is C₁-C₆ alkoxy; and at least one R⁷ is halo.

In some embodiments, o=1; p=2; R⁶ is C₁-C₆ alkoxy; and at least one R⁷ is chloro.

In some embodiments, at least one R⁷ is C₁-C₆ alkyl, and at least one R⁶ is C₁-C₆ alkyl optionally substituted with one or more halo.

In some embodiments, at least one R⁷ is isopropyl and at least one R⁶ is methyl.

In some embodiments, at least one R⁷ is C₁-C₆ alkyl, and at least one R⁶ is C₁-C₆ alkyl substituted with one or more halo.

In some embodiments, at least one R⁷ is isopropyl and at least one R⁶ is trifluoromethyl.

In some embodiments, at least one R⁷ is C₁-C₆ alkyl, and at least one R⁶ is C₃-C₇ cycloalkyl.

In some embodiments, at least one R⁷ is isopropyl and at least one R⁶ is cyclopropyl.

In some embodiments, o=1; p=1; R⁷ is isopropyl; and R⁶ is cyclopropyl.

In some embodiments, at least one R⁷ is C₁-C₆ alkyl, and at least one R⁶ is halo.

In some embodiments, at least one R⁷ is isopropyl and at least one R⁶ is halo.

In some embodiments, at least one R⁷ is isopropyl and at least one R⁶ is chloro.

In some embodiments, at least one R⁷ is isopropyl and at least one R⁶ is fluoro.

In some embodiments, o=1; p=1; R⁷ is isopropyl; and R⁶ is chloro.

In some embodiments, o=2; p=1; R⁷ is isopropyl; and at least one R⁶ is chloro.

In some embodiments, o=1; p=1; R⁷ is isopropyl; and R⁶ is fluoro.

In some embodiments, o=2; p=1; R⁷ is isopropyl; and at least one R⁶ is fluoro.

In some embodiments, o=2; p=2; R⁷ is isopropyl; and at least one R⁶ is fluoro.

In some embodiments, o=2; p=2; at least one R⁷ is isopropyl; one R⁶ is fluoro; and the other R⁶ is cyano.

In some embodiments, o=2; p=1; R⁷ is ethyl; and at least one R⁶ is fluoro.

In some embodiments, o=1; p=2; one R⁷ is isopropyl; the other R⁷ is trifluoromethyl; and R⁶ is chloro.

In some embodiments, at least one R⁷ is C₁-C₆ alkyl, and at least one R⁶ is cyano.

In some embodiments, at least one R⁷ is isopropyl and at least one R⁶ is cyano.

In some embodiments, o=1; p=1; R⁷ is isopropyl; and R⁶ is cyano.

In some embodiments, o=2; p=1; R⁷ is isopropyl; and at least one R⁶ is cyano.

In some embodiments, at least one R⁷ is C₃-C₇ cycloalkyl, and at least one R⁶ is C₃-C₇ cycloalkyl.

In some embodiments, at least one R⁷ is cyclopropyl, and at least one R⁶ is cyclopropyl.

In some embodiments, at least one R⁷ is C₃-C₇ cycloalkyl, and at least one R⁶ is halo.

In some embodiments, at least one R⁷ is cyclopropyl and at least one R⁶ is halo.

In some embodiments, at least one R⁷ is cyclopropyl and at least one R⁶ is chloro.

In some embodiments, at least one R⁷ is cyclopropyl and at least one R⁶ is fluoro.

In some embodiments, o=1; p=1; R⁷ is cyclopropyl; and R⁶ is chloro.

In some embodiments, o=1; p=1; R⁷ is cyclopropyl; and R⁶ is fluoro.

In some embodiments, at least one R⁷ is C₁-C₆ alkyl, and at least one R⁶ is C₁-C₆ alkoxy optionally substituted with one or more halo.

In some embodiments, at least one R⁷ is isopropyl, and at least one R⁶ is C₁-C₆ alkoxy.

In some embodiments, at least one R⁷ is isopropyl, and at least one R⁶ is methoxy.

In some embodiments, o=1; p=1; R⁷ is isopropyl, and R⁶ is methoxy.

In some embodiments, o=2; p=1; R⁷ is isopropyl, and at least one R⁶ is methoxy.

In some embodiments, at least one R⁷ is C₁-C₆ alkyl, and at least one R⁶ is C₁-C₆ alkoxy substituted with one or more halo.

In some embodiments, at least one R⁷ is isopropyl, and at least one R⁶ is trifluoromethoxy.

In some embodiments, at least one R⁷ is halo, and at least one R⁶ is C₁-C₆ haloalkyl optionally substituted with one or more hydroxy.

In some embodiments, o=1; p=1; R⁷ is chloro, and R⁶ is trifluoromethyl.

In some embodiments, at least one R⁷ is halo, and at least one R⁶ is C₁-C₆ haloalkoxy.

In some embodiments, at least one R⁷ is chloro, and at least one R⁶ is trifluoromethoxy.

In some embodiments, o=1; p=1; R⁷ is chloro, and R⁶ is trifluoromethoxy.

In some embodiments, at least one R⁷ is C₁-C₆ alkoxy; and at least one R⁶ is halo.

In some embodiments, o=1; p=2; at least one R⁷ is C₁-C₆ alkoxy; and R⁶ is chloro.

In some embodiments, R⁶ and R⁷ are each attached to a carbon of an aryl ring B.

In some embodiments, R⁶ and R⁷ are each attached to a carbon of a heteroaryl ring B.

In some embodiments, R⁶ is attached to a carbon and R⁷ is attached to a nitrogen of a heteroaryl ring B.

In some embodiments, R⁷ is attached to a carbon and R⁶ is attached to a nitrogen of a heteroaryl ring B.

In some embodiments, one R⁶ and one R⁷ are on adjacent atoms, and taken together with the atoms connecting them, form a C₅ carbocyclic ring optionally substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, R⁶ and R⁷ are on adjacent atoms, and taken together with the atoms connecting them, form a C₅ aliphatic carbocyclic ring.

In some embodiments, R⁶ and R⁷ are on adjacent atoms, and taken together with the atoms connecting them, form a C₆ carbocyclic ring optionally substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, R⁶ and R⁷ are on adjacent atoms, and taken together with the atoms connecting them, form a C₆ aliphatic carbocyclic ring.

In some embodiments, R⁶ and R⁷ are on adjacent atoms, and taken together with the atoms connecting them, form a C₆ aromatic carbocyclic ring.

In some embodiments, R⁶ and R⁷ are on adjacent atoms, and taken together with the atoms connecting them, form a 5-membered heterocyclic ring containing 1 or 2 heteroatoms independently selected from O, N, and S, optionally substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, R⁶ and R⁷ are on adjacent atoms, and taken together with the atoms connecting them, form a 5-membered aliphatic heterocyclic ring containing 1 or 2 heteroatoms independently selected from O, N, and S.

In some embodiments, R⁶ and R⁷ are on adjacent atoms, and taken together with the atoms connecting them, form a 5-membered heteroaromatic ring containing 1 or 2 heteroatoms independently selected from O, N, and S.

In some embodiments, R⁶ and R⁷ are on adjacent atoms, and taken together with the atoms connecting them, form a 6-membered heterocyclic ring containing 1 or 2 heteroatoms independently selected from O, N, and S, optionally substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, R⁶ and R⁷ are on adjacent atoms, and taken together with the atoms connecting them, form a 6-membered aliphatic heterocyclic ring containing 1 or 2 heteroatoms independently selected from O, N, and S.

In some embodiments, R⁶ and R⁷ are on adjacent atoms, and taken together with the atoms connecting them, form a 6-membered heteroaromatic ring containing 1 or 2 heteroatoms independently selected from O, N, and S.

In some embodiments, one R⁶ and one R⁷ are on adjacent atoms, and taken together with the atoms connecting them, form a C₄-C₈ carbocyclic ring or a 5- to 8-membered heterocyclic ring containing 1 or 2 heteroatoms independently selected from O, N, and S, wherein the ring is fused to the B ring at the 2- and 3- positions relative to the bond connecting the B ring to the NH(CO)group.

In some embodiments, o=1; p=2; and

one pair of one R⁶ and one R⁷, are on adjacent atoms; and said pair of one R⁶ and one R⁷ taken together with the atoms connecting them form form a C₄-C₈ carbocyclic ring or a 5- to 8-membered heterocyclic ring containing 1 or 2 heteroatoms independently selected from O, N, and S,

wherein the ring is fused to the B ring at the 2- and 3- positions relative to the bond connecting the B ring to the NR³(CO) group.

In some embodiments, o=1; p=2; and

one pair of one R⁶ and one R⁷, are on adjacent atoms; and said pair of one R⁶ and one R⁷ taken together with the atoms connecting them form form a C₄-C₈ carbocyclic ring optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, o=1; p=2; and

one pair of one R⁶ and one R⁷, are on adjacent atoms; and said pair of one R⁶ and one R⁷ taken together with the atoms connecting them form form a C₅ carbocyclic ring optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, o=1; p=2; and

one pair of one R⁶ and one R⁷, are on adjacent atoms; and said pair of one R⁶ and one R⁷ taken together with the atoms connecting them form form a C₅ aliphatic carbocyclic ring.

In some embodiments, o=2; p=2; and

one pair of one R⁶ and one R⁷, are on adjacent atoms; and said pair of one R⁶ and one R⁷ taken together with the atoms connecting them form form a C₄-C₈ carbocyclic ring optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹. In some embodiments, o=2; p=2; and

one pair of one R⁶ and one R⁷, are on adjacent atoms; and said pair of one R⁶ and one R⁷ taken together with the atoms connecting them form form a C₅ carbocyclic ring optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, o=1; p=2; and

one pair of one R⁶ and one R⁷, are on adjacent atoms; and said pair of one R⁶ and one R⁷ taken together with the atoms connecting them form form a C₅ aliphatic carbocyclic ring.

In some embodiments, o=2; p=2 or 3; and

two pairs, each of one R⁶ and one R⁷, are on adjacent atoms; one pair of one R⁶ and one R⁷ taken together with the atoms connecting them form a C₄ carbocyclic ring optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹; and the other pair of one R⁶ and one R⁷ taken together with the atoms connecting them form a C₅ carbocyclic ring optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, o=2; p=2 or 3; and

two pairs, each of one R⁶ and one R⁷, are on adjacent atoms; one pair of one R⁶ and one R⁷ taken together with the atoms connecting them form a C₄ aliphatic carbocyclic ring and the other pair of one R⁶ and one R⁷ taken together with the atoms connecting them form a C₅ aliphatic carbocyclic ring.

In some embodiments, o=2; p=2 or 3; and

two pairs, each of one R⁶ and one R⁷, are on adjacent atoms, and each pair of one R⁶ and one R⁷ taken together with the atoms connecting them form a C₅ carbocyclic ring optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, o=2; p=2 or 3; and

two pairs, each of one R⁶ and one R⁷, are on adjacent atoms, and each pair of one R⁶ and one R⁷ taken together with the atoms connecting them form a C₅ aliphatic carbocyclic ring.

In some embodiments, o=2; p=2 or 3; and

two pairs, each of one R⁶ and one R⁷, are on adjacent atoms, and each pair of one R⁶ and one R⁷ taken together with the atoms connecting them form a C₆ carbocyclic ring optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, o=2; p=2 or 3; and

two pairs, each of one R⁶ and one R⁷, are on adjacent atoms, and each pair of one R⁶ and one R⁷ taken together with the atoms connecting them form a C₆ aliphatic carbocyclic ring.

In some embodiments, o=2; p=2 or 3; and

two pairs, each of one R⁶ and one R⁷, are on adjacent atoms, and each pair of one R⁶ and one R⁷ taken together with the atoms connecting them form a C₆ aromatic carbocyclic ring.

In some embodiments, o=2; p=2 or 3; and

two pairs, each of one R⁶ and one R⁷, are on adjacent atoms, and each pair of one R⁶ and one R⁷ taken together with the atoms connecting them form a 5-membered heterocyclic ring containing 1 or 2 heteroatoms independently selected from O, N, and S, optionally substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, o=2; p=2 or 3; and

two pairs, each of one R⁶ and one R⁷, are on adjacent atoms, and each pair of one R⁶ and one R⁷ taken together with the atoms connecting them form a 5-membered aliphatic heterocyclic ring containing 1 or 2 heteroatoms independently selected from O, N, and S.

In some embodiments, o=2; p=2 or 3; and

two pairs, each of one R⁶ and one R⁷, are on adjacent atoms, and each pair of one R⁶ and one R⁷ taken together with the atoms connecting them form a 5-membered heteroaromatic ring containing 1 or 2 heteroatoms independently selected from O, N, and S.

In some embodiments, o=2; p=2 or 3; and

two pairs, each of one R⁶ and one R⁷, are on adjacent atoms, and each pair of one R⁶ and one R⁷ taken together with the atoms connecting them form a 6-membered heterocyclic ring containing 1 or 2 heteroatoms independently selected from O, N, and S, optionally substituted with one or more substituents independently selected from hydroxy, halo, oxo, C₁-C₆ alkyl, C₁-C₆ alkoxy, ═NR¹⁰, COOC₁-C₆ alkyl, C₆-C₁₀ aryl, and CONR⁸R⁹.

In some embodiments, o=2; p=2 or 3; and

two pairs, each of one R⁶ and one R⁷, are on adjacent atoms, and each pair of one R⁶ and one R⁷ taken together with the atoms connecting them form a 6-membered aliphatic heterocyclic ring containing 1 or 2 heteroatoms independently selected from O, N, and S.

In some embodiments, o=2; p=2 or 3; and

two pairs, each of one R⁶ and one R⁷, are on adjacent atoms, and each pair of one R⁶ and one R⁷ taken together with the atoms connecting them form a 6-membered heteroaromatic ring containing 1 or 2 heteroatoms independently selected from O, N, and S.

In some embodiments, o=2; p=2 or 3; and

two pairs, each of one R⁶ and one R⁷, are on adjacent atoms; one pair of one R⁶ and one R⁷ taken together with the atoms connecting them form a C₄-8 carbocyclic ring optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹; and the other pair of one R⁶ and one R⁷ taken together with the atoms connecting them form a 5- to 8-membered heterocyclic ring containing 1 or 2 heteroatoms independently selected from O, N, and S, optionally substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, o=2; p=2 or 3; and

two pairs, each of one R⁶ and one R⁷, are on adjacent atoms; one pair of one R⁶ and one R⁷ taken together with the atoms connecting them form a C₅ aliphatic carbocyclic ring and the other pair of one R⁶ and one R⁷ taken together with the atoms connecting them form a 5-membered aliphatic heterocyclic ring containing 1 or 2 heteroatoms independently selected from O, N, and S.

In some embodiments, o=2; p=2 or 3; and

two pairs, each of one R⁶ and one R⁷, are on adjacent atoms; one pair of one R⁶ and one R⁷ taken together with the atoms connecting them form a C5 carbocyclic ring optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹; and the other pair of one R⁶ and one R⁷ taken together with the atoms connecting them form a 6-membered heterocyclic ring containing 1 or 2 heteroatoms independently selected from O, N, and S, optionally substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, o=2; p=2 or 3; and

two pairs, each of one R⁶ and one R⁷, are on adjacent atoms; one pair of one R⁶ and one R⁷ taken together with the atoms connecting them form a C₅ aliphatic carbocyclic ring and the other pair of one R⁶ and one R⁷ taken together with the atoms connecting them form a 5-membered aliphatic heterocyclic ring containing 1 or 2 heteroatoms independently selected from O, N, and S.

In some embodiments, o=2; p=2 or 3; and

two pairs, each of one R⁶ and one R⁷, are on adjacent atoms, and each pair of one R⁶ and one R⁷ taken together with the atoms connecting them independently form a C₄-C₈ carbocyclic ring or a 5- to 8-membered heterocyclic ring containing 1 or 2 heteroatoms independently selected from O, N, and S,

wherein one of the two rings is fused to the B ring at the 2- and 3- positions relative to the bond connecting the B ring to the NR³(CO) group, and the other of the two rings is fused to the B ring at the 5- and 6- positions relative to the bond connecting the B ring to the NH(CO) group.

In some embodiments, o=2; p=2 or 3; and

two pairs, each of one R⁶ and one R⁷, are on adjacent atoms, and each pair of one R⁶ and one R⁷ taken together with the atoms connecting them independently form a C₄-C₈ carbocyclic ring or a 5- to 8-membered heterocyclic ring containing 1 or 2 heteroatoms independently selected from O, N, and S,

wherein one of the two rings is fused to the B ring at the 2- and 3-positions relative to the bond connecting the B ring to the NR³(CO) group, and the other of the two rings is fused to the B ring at the 4- and 5- positions relative to the bond connecting the B ring to the NH(CO) group.

In some embodiments, o=2; p=2; and

two pairs, each of one R⁶ and one R⁷, are on adjacent atoms, and each pair of one R⁶ and one R⁷ taken together with the atoms connecting them form a C5 aliphatic carbocyclic ring.

In some embodiments, o=2; p=2; and

two pairs, each of one R⁶ and one R⁷, are on adjacent atoms, one pair of one R⁶ and one R⁷ taken together with the atoms connecting them form a C₄ aliphatic carbocyclic ring, and the other pair of one R⁶ and one R⁷ taken together with the atoms connecting them form a C₅ aliphatic carbocyclic ring.

In some embodiments, o=2; p=2; and

two pairs, each of one R⁶ and one R⁷, are on adjacent atoms, and each pair of one R⁶ and one R⁷ taken together with the atoms connecting them form a C₄ aliphatic carbocyclic ring.

In some embodiments, o=2; p=2; and

two pairs, each of one R⁶ and one R⁷, are on adjacent atoms, one pair of one R⁶ and one R⁷ taken together with the atoms connecting them form a C₅ aliphatic carbocyclic ring, and the other pair of one R⁶ and one R⁷ taken together with the atoms connecting them form a 5-membered heterocyclic ring containing 1 or 2 heteroatoms independently selected from O, N, and S.

In some embodiments, o=2; p=3; and

two pairs, each of one R⁶ and one R⁷, are on adjacent atoms, and each pair of one R⁶ and one R⁷ taken together with the atoms connecting them form a C₅ aliphatic carbocyclic ring; and one R⁷ is halo (e.g., Cl or F).

In some embodiments, o=2; p=3; and

two pairs, each of one R⁶ and one R⁷, are on adjacent atoms, and each pair of one R⁶ and one R⁷ taken together with the atoms connecting them form a C₅ aliphatic carbocyclic ring; and one R⁷ is CN.

In some embodiments, one R⁷ is pyrazolyl and is para to the bond connecting the B ring to the NH(CO) group of Formula AA.

In some embodiments, one R⁷ is 3-pyrazolyl and is para to the bond connecting the B ring to the NH(CO) group of Formula AA.

In some embodiments, one R⁷ is 4-pyrazolyl and is para to the bond connecting the B ring to the NH(CO) group of Formula AA.

In some embodiments, one R⁷ is 5-pyrazolyl and is para to the bond connecting the B ring to the NH(CO) group of Formula AA.

In some embodiments, one R⁷ is thiazolyl and is para to the bond connecting the B ring to the NH(CO) group of Formula AA.

In some embodiments, one R⁷ is 4-thiazolyl and is para to the bond connecting the B ring to the NH(CO) group of Formula AA.

In some embodiments, one R⁷ is 5-thiazolyl and is para to the bond connecting the B ring to the NH(CO) group of Formula AA.

In some embodiments, one R⁷ is furyl and is para to the bond connecting the B ring to the NH(CO) group of Formula AA.

In some embodiments, one R⁷ is 2-furyl and is para to the bond connecting the B ring to the NH(CO) group of Formula AA.

In some embodiments, one R⁷ is thiophenyl and is para to the bond connecting the B ring to the NH(CO) group of Formula AA.

In some embodiments, one R⁷ is 2-thiophenyl and is para to the bond connecting the B ring to the NH(CO) group of Formula AA.

In some embodiments, one R⁷ is phenyl and is para to the bond connecting the B ring to the NH(CO) group of Formula AA.

In some embodiments, one R⁷ is cycloalkenyl (e.g., cyclopentenyl, e.g., 1-cyclopentenyl) and is para to the bond connecting the B ring to the NR³(CO) group of Formula AA.

In some embodiments, one R⁷ is phenyl optionally substituted with one or more C₁-C₆ alkyl (e.g., methyl or propyl, e.g., 2-propyl) optionally substituted with one or more hydroxyl, NR⁸R⁹ (e.g., dimethylamino), or C₆-C₁₀ aryl (e.g., phenyl, naphthyl, or methylenedioxyphenyl and is para to the bond connecting the B ring to the NH(CO) group of Formula AA.

In some embodiments, one R⁷ is phenyl optionally substituted with one or more C₁-C₆ alkoxy (e.g., methoxy) optionally substituted with one or more hydroxyl, NR⁸R⁹ (e.g., dimethylamino), or C₆-C₁₀ aryl (e.g., phenyl, naphthyl, or methylenedioxyphenyl and is para to the bond connecting the B ring to the NH(CO) group of Formula AA.

In some embodiments, one R⁷ is phenyl optionally substituted with one or more C₆-C₁₀ aryloxy (e.g., phenoxy) and is para to the bond connecting the B ring to the NH(CO) group of Formula AA.

In some embodiments, one R⁷ is phenyl optionally substituted with one or more CN and is para to the bond connecting the B ring to the NH(CO) group of Formula AA.

In some embodiments, one R⁷ is phenyl optionally substituted with one or more halo (e.g., F, Cl) and is para to the bond connecting the B ring to the NH(CO) group of Formula AA and is para to the bond connecting the B ring to the NH(CO) group of Formula AA.

In some embodiments, one R⁷ is phenyl optionally substituted with one or more COOC₁-C₆ alkyl (e.g., CO₂t-Bu) and is para to the bond connecting the B ring to the NH(CO) group of Formula AA.

In some embodiments, one R⁷ is phenyl optionally substituted with one or more S(O₂)C₁-C₆ alkyl (e.g., S(O₂)methyl) and is para to the bond connecting the B ring to the NH(CO) group of Formula AA.

In some embodiments, one R⁷ is phenyl optionally substituted with one or more 3- to 7-membered heterocycloalkyl (e.g., morpholinyl) and is para to the bond connecting the B ring to the NH(CO) group of Formula AA.

In some embodiments, one R⁷ is phenyl optionally substituted with one or more CONR⁸R⁹ (e.g., unsubstituted amido) and is para to the bond connecting the B ring to the NH(CO) group of Formula AA.

In some embodiments, one R⁷ is phenyl optionally substituted with one or more C₁-C₆ alkyl (e.g., methyl or propyl, e.g., 2-propyl) and with one or more halo (e.g., F, Cl) and is para to the bond connecting the B ring to the NH(CO) group of Formula AA and is para to the bond connecting the B ring to the NH(CO) group of Formula AA.

In some embodiments, R⁶ and R⁷ are each attached to a carbon of an aryl ring B.

In some embodiments, R⁶ and R⁷ are each attached to a carbon of a heteroaryl ring B.

In some embodiments, R⁶ is attached to a carbon and R⁷ is attached to a nitrogen of a heteroaryl ring B.

In some embodiments, R⁷ is attached to a carbon and R⁶ is attached to a nitrogen of a heteroaryl ring B.

In some embodiments, R⁶ and R⁷ are each independently selected from a C₂-C₆ alkyl, C₂-C₆ haloalkyl, C₂-C₆ alkoxy, C₁-C₆ haloalkoxy, I, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, C₃-C₁₀ cycloalkyl, and 3- to 10-membered heterocycloalkyl, and C₂-C₆ alkenyl,

wherein R⁶ and R⁷ are each optionally substituted with one or more substituents independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), NHCOC₂-C₆ alkynyl, C₆-C₁₀ aryloxy, and S(O₂)C₁-C₆ alkyl; and wherein the C₁-C₆ alkyl or C₁-C₆ alkoxy that R⁶ or R⁷ is substituted with is optionally substituted with one or more hydroxyl, C₆-C₁₀ aryl or NR⁸R⁹, or wherein R⁶ or R⁷ is optionally fused to a five- to -seven-membered carbocyclic ring or heterocyclic ring containing one or two heteroatoms independently selected from oxygen, sulfur and nitrogen;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl), and NHCO(3- to 7-membered heterocycloalkyl) are optionally substituted with one or more substituents independently selected from halo, C₁-C₆ alkyl, and OC₁-C₆ alkyl;

or one pair of R⁶ and R⁷ on adjacent atoms, taken together with the atoms connecting them, independently form at least one C₄-C₈ carbocyclic ring or at least one 5- to 8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹.

In some embodiments, R⁶ and R⁷ are each independently selected from a C₂-C₆ alkyl, C₂-C₆ haloalkyl, C₂-C₆ alkoxy, C₁-C₆ haloalkoxy, I, CN, NO₂, COC₁-C₆ alkyl, CO₂C₁-C₆ alkyl, CO₂C₃-C₅ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, C₃-C₁₀ cycloalkyl and 3- to 10-membered heterocycloalkyl, and C₂-C₆ alkenyl,

wherein R⁶ and R⁷ are each optionally substituted with one or more substituents independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), NHCOC₂-C₆ alkynyl, C₆-C₁₀ aryloxy, and S(O₂)C₁-C₆ alkyl; and wherein the C₁-C₆ alkyl or C₁-C₆ alkoxy that R⁶ or R⁷ is substituted with is optionally substituted with one or more hydroxyl, C₆-C₁₀ aryl or NR⁸R⁹, or wherein R⁶ or R⁷ is optionally fused to a five- to -seven-membered carbocyclic ring or heterocyclic ring containing one or two heteroatoms independently selected from oxygen, sulfur and nitrogen;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;

or one pair of R⁶ and R⁷ on adjacent atoms, taken together with the atoms connecting them, independently form at least one C₄-C₈ carbocyclic ring or at least one 5- to 8-membered heterocyclic ring containing 1 or 2 heteroatoms and/or heteroatomic groups independently selected from O, NH, NR¹³, S, S(O), and S(O)₂, wherein the selected heteroatoms and/or heteroatomic groups are cumulative with the nitrogen atoms present in ring B, and wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹.

In some embodiments, R⁶ and R⁷ are each independently selected from a C₂-C₆ alkyl, C₂-C₆ haloalkyl, C₁-C₆ haloalkoxy, I, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, C₃-C₁₀ cycloalkyl and 3- to 10-membered heterocycloalkyl, and C₂-C₆ alkenyl,

wherein R⁶ and R⁷ are each optionally substituted with one or more substituents independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), NHCOC₂-C₆ alkynyl, C₆-C₁₀ aryloxy, and S(O₂)C₁-C₆ alkyl; and wherein the C₁-C₆ alkyl or C₁-C₆ alkoxy that R⁶ or R⁷ is substituted with is optionally substituted with one or more hydroxyl, C₆-C₁₀ aryl or NR⁸R⁹, or wherein R⁶ or R⁷ is optionally fused to a five- to -seven-membered carbocyclic ring or heterocyclic ring containing one or two heteroatoms independently selected from oxygen, sulfur and nitrogen;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;

or one pair of R⁶ and R⁷ on adjacent atoms, taken together with the atoms connecting them, independently form at least one C₄-C₈ carbocyclic ring or at least one 5- to 8-membered heterocyclic ring containing 1 or 2 heteroatoms and/or heteroatomic groups independently selected from O, NH, NR¹³, S, S(O), and S(O)₂, wherein the selected heteroatoms and/or heteroatomic groups are cumulative with the nitrogen atoms present in ring B, and wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹.

In some embodiments, R⁶ and R⁷ are each independently selected from a C₂-C₆ alkyl, C₂-C₆ haloalkyl, C₂-C₆ alkoxy, C₁-C₆ haloalkoxy, I, CN, NO₂, COC₁-C₆ alkyl, CO₂C₁-C₆ alkyl, CO₂C₃-C₅ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, C₃-C₁₀ cycloalkyl and 3- to 10-membered heterocycloalkyl, and C₂-C₆ alkenyl,

wherein R⁶ and R⁷ are each optionally substituted with one or more substituents independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), NHCOC₂-C₆ alkynyl, C₆-C₁₀ aryloxy, and S(O₂)C₁-C₆ alkyl; and wherein the C₁-C₆ alkyl or C₁-C₆ alkoxy that R⁶ or R⁷ is substituted with is optionally substituted with one or more hydroxyl, C₆-C₁₀ aryl or NR⁸R⁹, or wherein R⁶ or R⁷ is optionally fused to a five- to -seven-membered carbocyclic ring or heterocyclic ring containing one or two heteroatoms independently selected from oxygen, sulfur and nitrogen;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;

or one pair of R⁶ and R⁷ on adjacent atoms, taken together with the atoms connecting them, independently form at least one C₄-C₈ carbocyclic ring or at least one 5- to 8-membered heterocyclic ring containing 1 or 2 heteroatoms and/or heteroatomic groups independently selected from O, NH, NR¹³, S, S(O), and S(O)₂, wherein the selected heteroatoms and/or heteroatomic groups are cumulative with the nitrogen atoms present in ring B, and wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹.

In some embodiments, B is

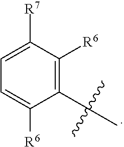

In some embodiments, B is

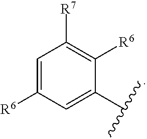

In some embodiments, B is

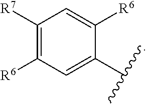

In certain embodiments (when B is

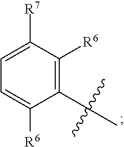

### B is

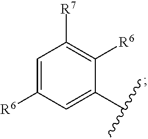

### or B is

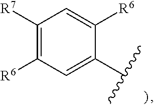

each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO-C₁-C₆ alkyl; CONR⁸R⁹, and 4- to 6-membered heterocycloalkyl,

wherein the C₁-C₆ alkyl, C₁-C₆ haloalkyl, C₆-C₁₀ aryl, 5- to 10-membered heteroaryl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;

wherein R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy;

or R⁶ and R⁷, taken together with the atoms connecting them, independently form C₄-C₇ carbocyclic ring or at least one 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, B is

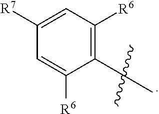

In certain of these embodiments, each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO-C₁-C₆ alkyl; CONR⁸R⁹, and 4- to 6-membered heterocycloalkyl, wherein the C₁-C₆ alkyl, C₁-C₆ haloalkyl, C₆-C₁₀ aryl, 5- to 10-membered heteroaryl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;

wherein R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy.

In certain of these embodiments, the other R⁶ is C₁-C₆ alkyl. For example, each R⁶ is isopropyl (i.e., the substituted ring B is

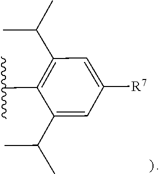

In certain other embodiments (when the substituted ring B is

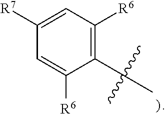

one R⁶ is C₁-C₆ alkyl; and the other R⁶ is C₆-C₁₀ aryl or 5- to 10-membered heteroaryl, each of which is optionally substituted with one or more substituents each independently selected from: hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl.

In certain of these embodiments, one R⁶ is C₁-C₆ alkyl; and the other R⁶ is C₆-C₁₀ aryl or 5- to 10-membered heteroaryl optionally substituted with a substituent selected from halo, CN, C₁-C₆ alkyl, and C₁-C₆ alkoxy. For example, R⁶ is 5-6 (e.g., 6) membered heteroaryl (e.g., pyridinyl (e.g., pyridin-4-yl), pyrimidinyl, or thiazolyl) optionally substituted with a substituent selected from hydroxyl, halo, CN, C₁-C₆ alkyl, and C₁-C₆ alkoxy.

As a non-limiting example of the foregoing embodiments, substituted ring B is selected from:

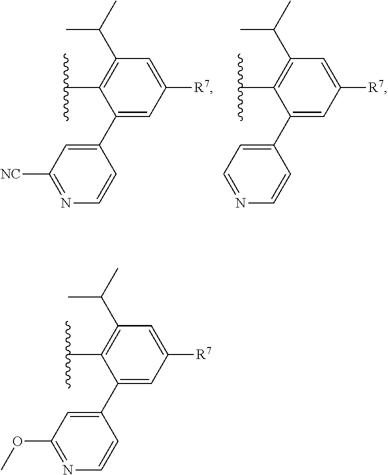

(e.g., R⁷ is halo (e.g., fluoro)).

In some embodiments, B is

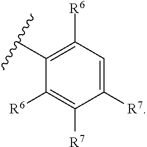

In certain of these embodiments, one R⁶ is C₁-C₆ alkyl; and the other R⁶ is C₆-C₁₀ aryl or 5- to 10-membered heteroaryl, each of which is optionally substituted with one or more substituents each independently selected from: hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl.

In certain of these embodiments, one R⁶ is C₁-C₆ alkyl; and the other R⁶ is C₆-C₁₀ aryl or 5- to 10-membered heteroaryl optionally substituted with a substituent selected from halo, CN, C₁-C₆ alkyl, and C₁-C₆ alkoxy. For example, R⁶ is 5-6 (e.g., 6) membered heteroaryl (e.g., pyridinyl (e.g., pyridin-4-yl), pyrimidinyl, or thiazolyl) optionally substituted with a substituent selected from hydroxyl, halo, CN, C₁-C₆ alkyl, and C₁-C₆ alkoxy.

In certain of the foregoing embodiments, each R⁷ is independently halo or cyano,

As a non-limiting example of the foregoing embodiments, substituted ring B is:

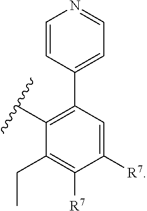

In some embodiments, B is

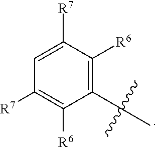

In some embodiments, B is

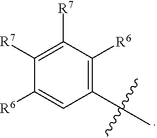

In certain embodiments (when B is

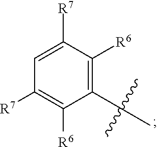

### or B is

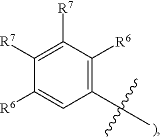

each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO-C₁-C₆ alkyl; CONR⁸R⁹, and 4- to 6-membered heterocycloalkyl,

wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;

wherein each R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy;

or at least one pair of R⁶ and R⁷ on adjacent atoms, taken together with the atoms connecting them, independently form at least one C₄-C₇ carbocyclic ring or at least one 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, ═NR¹⁰, COOC₁-C₆ alkyl, C₆-C₁₀ aryl, and CONR⁸R⁹.

In certain embodiments (when B is

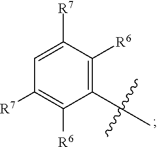

### or B is

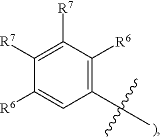

two pairs, each of one R⁶ and one R⁷, are on adjacent atoms, and each pair of one R⁶ and one R⁷ taken together with the atoms connecting them independently form a C₄-C₇ carbocyclic ring or a 5- to 7-membered heterocyclic ring containing 1 or 2 heteroatoms independently selected from O, N, and S, wherein each carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In certain of the foregoing embodiments, one pair of R⁶ and R⁷ taken together with the atoms connecting them independently form a C₅ carbocyclic ring, wherein the carbocyclic ring is optionally substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In certain of these embodiments (when one pair of R⁶ and R⁷ taken together with the atoms connecting them independently form a C₅ carbocyclic ring, wherein the carbocyclic ring is optionally substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹), the other pair of R⁶ and R⁷ taken together with the atoms connecting them independently form a C₅ carbocyclic ring, wherein the carbocyclic ring is optionally substituted with one or more substituents independently selected from hydroxy, halo, oxo, C₁-C₆ alkyl, C₁-C₆ alkoxy, ═NR¹⁰, COOC₁-C₆ alkyl, C₆-C₁₀ aryl, and CONR⁸R⁹.

As non-limiting examples of the foregoing embodiments (when B is

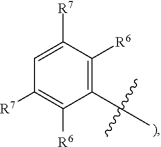

the substituted ring B is:

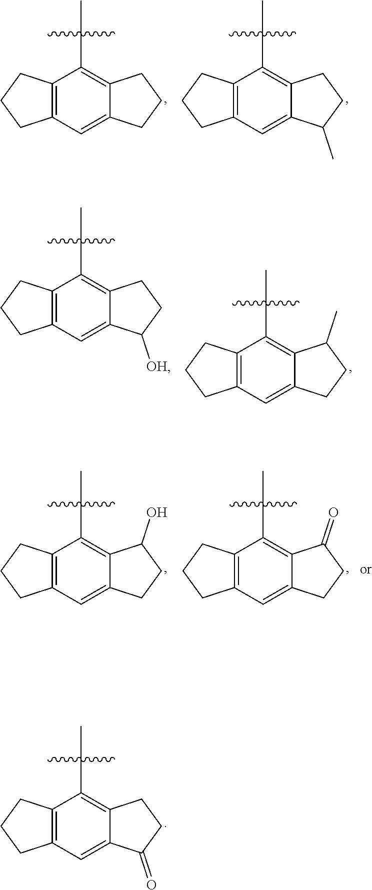

As a non-limiting example of the foregoing embodiments (when B is

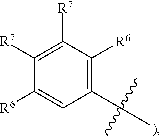

the substituted ring B is:

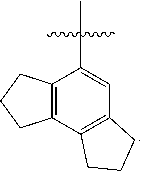 [SMILES: *c1cc2c(c3c1CCC3)CCC2]

In certain of these embodiments (when o=2 and p=2 (e.g., B is

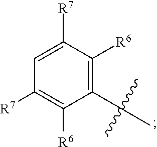

### or B is

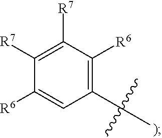

and one pair of R⁶ and R⁷ taken together with the atoms connecting them independently form a C₅ carbocyclic ring, wherein the carbocyclic ring is optionally substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹),

the other pair of R⁶ and R⁷ taken together with the atoms connecting them independently form a C₄ or C₆₋₇ (e.g., C₄) carbocyclic ring, wherein the carbocyclic ring is optionally substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

As a non-limiting example, the substituted ring B is:

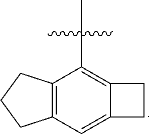 [SMILES: *c1c2c(cc3c1CC3)CCC2]

In certain of these embodiments (when o=2 and p=2 (e.g., B is

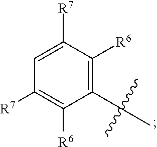

### or B is

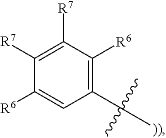

one pair of R⁶ and R⁷ taken together with the atoms connecting them independently form a C₄ carbocyclic ring, wherein the carbocyclic ring is optionally substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In certain of these embodiments, the other pair of R⁶ and R⁷ taken together with the atoms connecting them independently form a C₄ carbocyclic ring, wherein the carbocyclic ring is optionally substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments (when the substituted ring B is

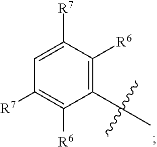

and each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO-C₁-C₆ alkyl; CONR⁸R⁹, and 4- to 6-membered heterocycloalkyl,

wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;

wherein each R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy;

or at least one pair of R⁶ and R⁷ on adjacent atoms, taken together with the atoms connecting them, independently form at least one C₄-C₇ carbocyclic ring or at least one 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹),

one pair of R⁶ and R⁷ on adjacent atoms, taken together with the atoms connecting them, independently form one C₄-C₇ carbocyclic ring or one 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹; and each of the remaining R⁶ and R⁷ is independently selected from C₁-C₆ alkyl and C₃-C₇ cycloalkyl.

In certain of these embodiments, one pair of R⁶ and R⁷ on adjacent atoms, taken together with the atoms connecting them, independently form one C₄-C₇ (e.g., C₅) carbocyclic ring; and each of the remaining R⁶ and R⁷ is independently selected from C₁-C₆ alkyl and C₃-C₇ cycloalkyl.

As a non-limiting example of the foregoing embodiments, the substituted ring B is

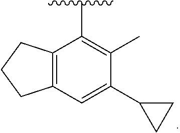 [SMILES: *c1c(C)c(C2CC2)cc2c1CCC2]

As a non-limiting example, the substituted ring B is:

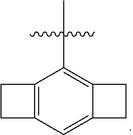 [SMILES: *c1c2c(cc3c1CC3)CC2]

In some embodiments, B is

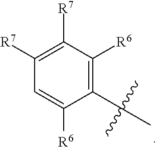

In certain of these embodiments, each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO-C₁-C₆ alkyl; CONR⁸R⁹, and 4- to 6-membered heterocycloalkyl, wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;

wherein each R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy;

or R⁶ and R⁷, taken together with the atoms connecting them, independently form C₄-C₇ carbocyclic ring or at least one 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, B is

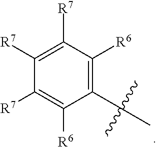

In certain of these embodiments, each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO-C₁-C₆ alkyl; CONR⁸R⁹, and 4- to 6-membered heterocycloalkyl,

wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;

wherein each R⁷ is independently selected from C₁-C₆ alkyl, C₂-C₆ alkenyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy;

or at least one pair of R⁶ and R⁷ on adjacent atoms, taken together with the atoms connecting them, independently form at least one C₄-C₇ carbocyclic ring or at least one 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, ═NR¹⁰ COOC₁-C₆ alkyl, C₆-C₁₀ aryl, and CONR⁸R⁹.

In certain of the foregoing embodiments, two pairs, each of one R⁶ and one R⁷, are on adjacent atoms, and each pair of one R⁶ and one R⁷ taken together with the atoms connecting them independently form a C₄-C₇ carbocyclic ring or a 5- to 7-membered heterocyclic ring containing 1 or 2 heteroatoms independently selected from O, N, and S, wherein each carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In certain of these embodiments, one pair of R⁶ and R⁷ taken together with the atoms connecting them independently form a C₅ carbocyclic ring, wherein the carbocyclic ring is optionally substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In certain of the foregoing embodiments, the other pair of R⁶ and R⁷ taken together with the atoms connecting them independently form a C₅ carbocyclic ring, wherein the carbocyclic ring is optionally substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

As a non-limiting example of the foregoing embodiments, the substituted ring B is:

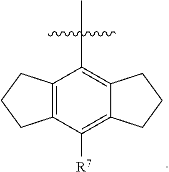

For example, R⁷ is selected from each R⁷ is independently selected from C₁-C₆ alkyl, C₂-C₆ alkenyl, C₁-C₆ haloalkyl, C₁-C₆ alkoxy, C₁-C₆ haloalkoxy, halo, and CN.

In some embodiments, the substituted ring B is selected from:

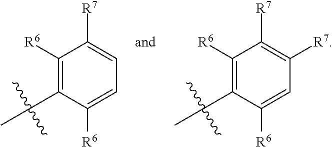

In certain of the foregoing embodiments, each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO-C₁-C₆ alkyl, CONR⁸R⁹, and 4- to 6-membered heterocycloalkyl,

wherein each of the C₁-C₆ alkyl, C₁-C₆ haloalkyl, C₃-C₇ cycloalkyl, 4- to 6-membered heterocycloalkyl, C₆-C₁₀ aryl, and 5- to 10-membered heteroaryl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;

wherein R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₂-C₆ alkynyl, C₃-C₇ cycloalkyl and 4- to 6-membered heterocycloalkyl, wherein each of the C₂-C₆ alkynyl and C₁-C₆ alkyl is optionally substituted with from 1-2 substituents each independently selected from oxo, C₁-C₆ alkoxy, C₃-C₁₀ cycloalkyl, 3- to 7-membered heterocycloalkyl, and C₃-C₁₀ cycloalkoxy;

or R⁶ and R⁷ on adjacent atoms, taken together with the atoms connecting them, independently form C₄-C₇ carbocyclic ring or 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In certain of these embodiments, the R⁶ and R⁷ on adjacent atoms, taken together with the atoms connecting them, independently form C₄-C₇ (e.g., C₄ or C₅) carbocyclic ring or 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹. For example, the R⁶ and R⁷ on adjacent atoms, taken together with the atoms connecting them, independently form C₄-C₇ (e.g., C₅) carbocyclic ring. For example, the substituted ring B is

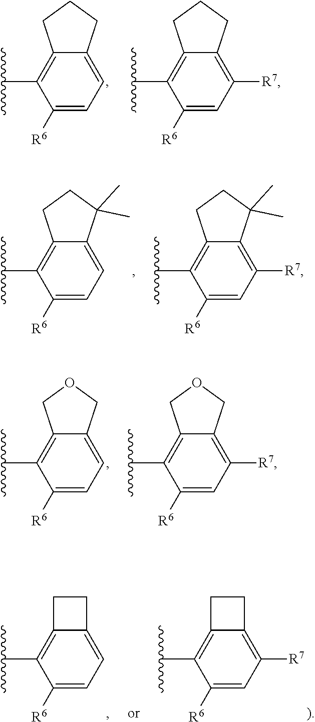

In certain embodiments (when the substituted ring B is selected from:

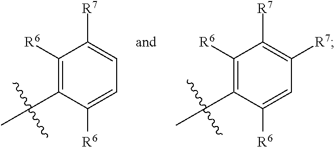

and the R⁶ and R⁷ on adjacent atoms, taken together with the atoms connecting them, independently form C₄-C₇ (e.g., C₄ or C₅) carbocyclic ring or 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, ═NR¹⁰, COOC₁-C₆ alkyl, C₆-C₁₀ aryl, and CONR⁸R⁹):

the remaining R⁶ is C₆-C₁₀ aryl or 5- to 10-membered heteroaryl, each of which is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl.

In certain of these embodiments, the remaining R⁶ is C₆-C₁₀ aryl or 5- to 10-membered heteroaryl optionally substituted with a substituent selected from halo, CN, C₁-C₆ alkyl, and C₁-C₆ alkoxy. For example, R⁶ is 5-6 membered heteroaryl (e.g., pyridinyl (e.g., pyridin-4-yl), pyrimidinyl, or thiazolyl) optionally substituted with a substituent selected from halo, CN, C₁-C₆ alkyl, and C₁-C₆ alkoxy.

As a non-limiting example of the foregoing embodiments, substituted ring B is selected from:

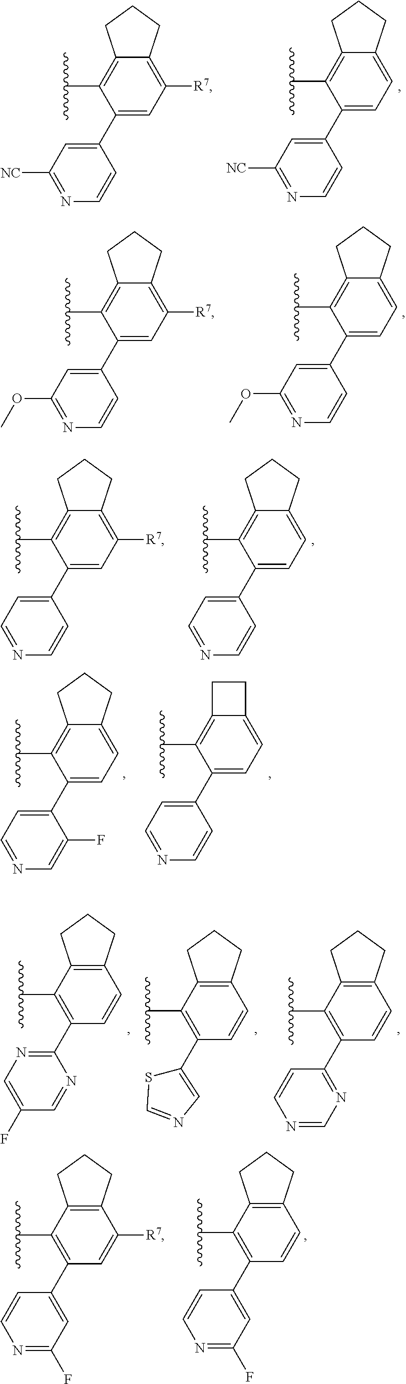

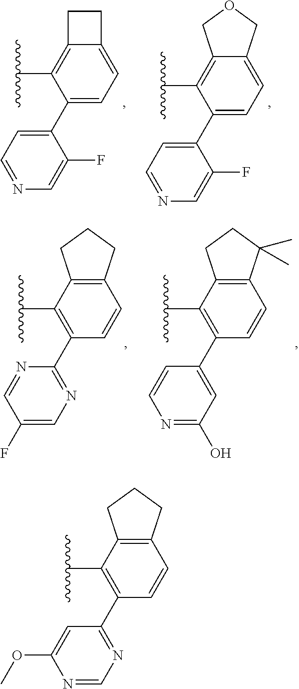

(e.g., R⁷ is halo (e.g., fluoro)).

In some embodiments, the substituted ring B is selected from:

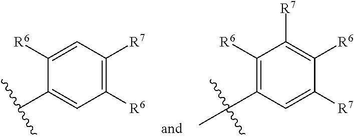

In certain of the foregoing embodiments, each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO-C₁-C₆ alkyl, CONR⁸R⁹, and 4- to 6-membered heterocycloalkyl,

wherein each of the C₁-C₆ alkyl, C₁-C₆ haloalkyl, C₃-C₇ cycloalkyl, 4- to 6-membered heterocycloalkyl, C₆-C₁₀ aryl, and 5- to 10-membered heteroaryl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;

wherein R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₂-C₆ alkynyl, C₃-C₇ cycloalkyl and 4- to 6-membered heterocycloalkyl, wherein each of the C₂-C₆ alkynyl and C₁-C₆ alkyl is optionally substituted with from 1-2 substituents each independently selected from oxo, C₁-C₆ alkoxy, C₃-C₁₀ cycloalkyl, 3- to 7-membered heterocycloalkyl, and C₃-C₁₀ cycloalkoxy;

or one pair R⁶ and R⁷ on adjacent atoms, taken together with the atoms connecting them, independently form C₄-C₇ carbocyclic ring or 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In certain of these embodiments, one pair R⁶ and R⁷ on adjacent atoms, taken together with the atoms connecting them, independently form C₄-C₇ (e.g., C₄ or C₅) carbocyclic ring or 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹. For example, the R⁶ and R⁷ on adjacent atoms, taken together with the atoms connecting them, independently form C₄-C₇ (e.g., C₅) carbocyclic ring. For example, the substituted ring B is:

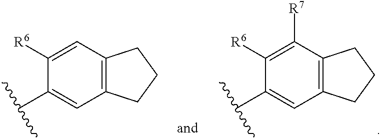

In certain embodiments (when the substituted ring B is selected from:

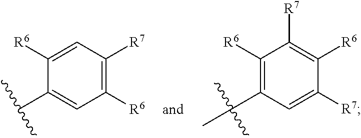

and one pair of R⁶ and R⁷ on adjacent atoms, taken together with the atoms connecting them, independently form C₄-C₇ (e.g., C₄ or C₅) carbocyclic ring or 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹):

the remaining R⁶ is C₆-C₁₀ aryl or 5- to 10-membered heteroaryl, each of which is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl.

In certain of these embodiments, the remaining R⁶ is C₆-C₁₀ aryl or 5- to 10-membered heteroaryl optionally substituted with a substituent selected from halo, CN, C₁-C₆ alkyl, and C₁-C₆ alkoxy. For example, R⁶ is 5-6 membered heteroaryl (e.g., pyridinyl (e.g., pyridin-4-yl), pyrimidinyl, or thiazolyl) optionally substituted with a substituent selected from halo, CN, C₁-C₆ alkyl, and C₁-C₆ alkoxy.

As a non-limiting example of the foregoing embodiments, substituted ring B is selected from:

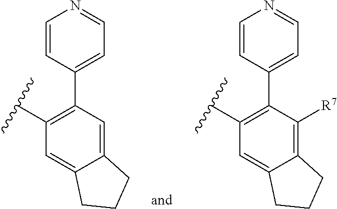

(e.g., R⁷ is halo (e.g., fluoro)).

In some embodiments, B is

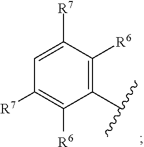

and (BB-2) applies.

In some embodiments, B is

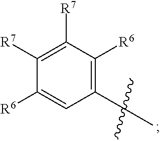

and (BB-2) applies.

In certain embodiments (when B is

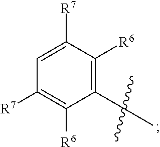

### or B is

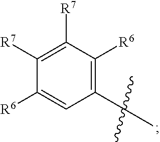

and when (BB-2) applies), each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO-C₁-C₆ alkyl; CONR⁸R⁹, and 4- to 6-membered heterocycloalkyl,

wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;

wherein each R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy;

or at least one pair of R⁶ and R⁷ on adjacent atoms, taken together with the atoms connecting them, independently form at least one C₄-C₇ carbocyclic ring or at least one 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, ═NR¹⁰, COOC₁-C₆ alkyl, C₆-C₁₀ aryl, and CONR⁸R⁹, provided that:

when two pairs of R⁶ and R⁷ are on adjacent atoms; and each pair taken together with the atoms connecting them, independently forms a ring selected from a C₄-C₈ carbocyclic ring and a 5- to 8-membered heterocyclic ring containing 1 or 2 heteroatoms independently selected from O, N, and S,

then at least one ring is selected from:

(a) a C₄ carbocyclic ring, a C₆-C₈ carbocyclic ring, or a 5- to 8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹; and

(b) a C₅ carbocyclic ring substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹.

In certain of the forgoing embodiments, two pairs, each of one R⁶ and one R⁷, are on adjacent atoms, and each pair of one R⁶ and one R⁷ taken together with the atoms connecting them independently form a C₄-C₇ carbocyclic ring or a 5- to 7-membered heterocyclic ring containing 1 or 2 heteroatoms independently selected from O, N, and S, wherein each carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In certain of the foregoing embodiments, one pair of R⁶ and R⁷ taken together with the atoms connecting them independently form a C₅ carbocyclic ring, wherein the carbocyclic ring is optionally substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In certain of these embodiments (when one pair of R⁶ and R⁷ taken together with the atoms connecting them independently form a C₅ carbocyclic ring, wherein the carbocyclic ring is optionally substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹), the other pair of R⁶ and R⁷ taken together with the atoms connecting them independently form a C₅ carbocyclic ring, wherein the carbocyclic ring is optionally substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

As non-limiting examples of the foregoing embodiments (when B is

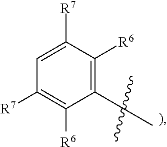

the substituted ring B is:

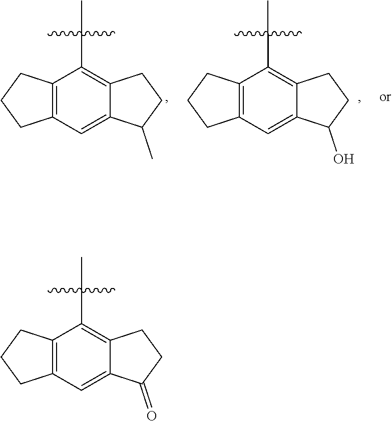

As additional non-limiting examples of the foregoing embodiments (when B is

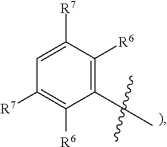

the substituted ring B is:

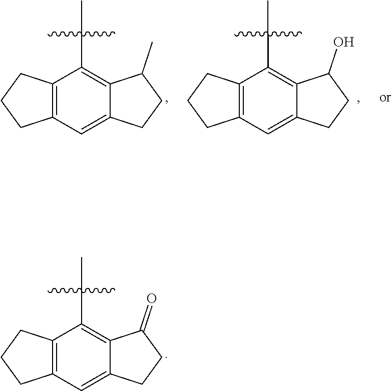

As a non-limiting example of the foregoing embodiments (when B is

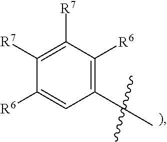

the substituted ring B is:

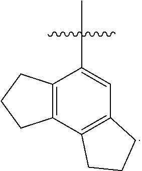 [SMILES: *c1cc2c(c3c1CCC3)CCC2]

In certain of these embodiments (when o=2 and p=2 (e.g., B is

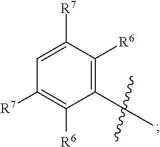

### or B is

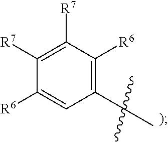

and one pair of R⁶ and R⁷ taken together with the atoms connecting them independently form a C₅ carbocyclic ring, wherein the carbocyclic ring is optionally substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹),

the other pair of R⁶ and R⁷ taken together with the atoms connecting them independently form a C₄ or C₆₋₇ (e.g., C₄) carbocyclic ring, wherein the carbocyclic ring is optionally substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

As a non-limiting example, the substituted ring B is:

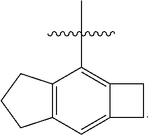 [SMILES: *c1c2c(cc3c1CC3)CCC2]

In certain of these embodiments (when o=2 and p=2 (e.g., B is

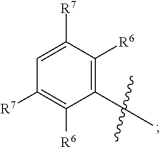

### or B is

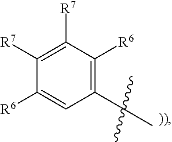

one pair of R⁶ and R⁷ taken together with the atoms connecting them independently form a C₄ carbocyclic ring, wherein the carbocyclic ring is optionally substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In certain of these embodiments, the other pair of R⁶ and R⁷ taken together with the atoms connecting them independently form a C₄ carbocyclic ring, wherein the carbocyclic ring is optionally substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments (when the substituted ring B is

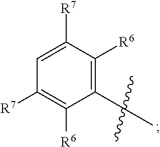

and each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO-C₁-C₆ alkyl; CONR⁸R⁹, and 4- to 6-membered heterocycloalkyl,

wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;

wherein each R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy;

or at least one pair of R⁶ and R⁷ on adjacent atoms, taken together with the atoms connecting them, independently form at least one C₄-C₇ carbocyclic ring or at least one 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹),

one pair of R⁶ and R⁷ on adjacent atoms, taken together with the atoms connecting them, independently form one C₄-C₇ carbocyclic ring or one 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹; and each of the remaining R⁶ and R⁷ is independently selected from C₁-C₆ alkyl and C₃-C₇ cycloalkyl.

In certain of these embodiments, one pair of R⁶ and R⁷ on adjacent atoms, taken together with the atoms connecting them, independently form one C₄-C₇ (e.g., C₅) carbocyclic ring; and each of the remaining R⁶ and R⁷ is independently selected from C₁-C₆ alkyl and C₃-C₇ cycloalkyl.

As a non-limiting example of the foregoing embodiments, the substituted ring B is

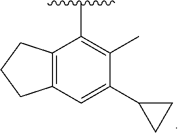 [SMILES: *c1c(C)c(C2CC2)cc2c1CCC2]

As a non-limiting example, the substituted ring B is:

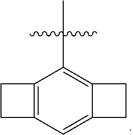 [SMILES: *c1c2c(cc3c1CC3)CC2]

In some embodiments, B is 3-pyridyl.

In some embodiments (when B is 3-pyridyl), o=2 and p=1.

In certain embodiments, the substituted ring B is

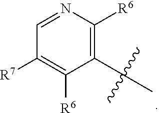

In certain of these embodiments, each R⁶ is independently selected from the group consisting of: C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO-C₁-C₆ alkyl; CONR⁸R⁹, and 4- to 6-membered heterocycloalkyl, wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl.

In certain of the foregoing embodiments, each R⁶ is independently selected from the group consisting of: C₁-C₆ alkyl, halo, C₃-C₇ cycloalkyl, and C₆-C₁₀ aryl.

As non-limiting examples of the foregoing embodiments, each R⁶ is independently selected from the group consisting of: methyl, isopropyl, cyclopropyl, fluoro, and phenyl.

In certain of any of the foregoing embodiments (when the substituted ring B is

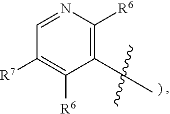

each R⁷ is independently selected from the group consisting of: C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO-C₁-C₆ alkyl; CONR⁸R⁹, and 4- to 6-membered heterocycloalkyl, wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl.

In certain of these embodiments, each R⁷ is independently selected from the group consisting of: C₁-C₆ alkyl, C₃-C₇ cycloalkyl, halo, and C₆-C₁₀ aryl.

As non-limiting examples of the foregoing embodiments, each R⁷ is independently selected from the group consisting of: methyl, isopropyl, cyclopropyl, fluoro, and phenyl.

In some embodiments, the substituted ring B is 4-pyridyl.

In certain of these embodiments, o=2 and p=0.

In certain of the foregoing embodiments, the substituted ring B is

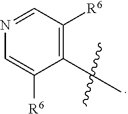

In certain of these embodiments, each R⁶ is independently selected from the group consisting of: C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO-C₁-C₆ alkyl; CONR⁸R⁹, and 4- to 6-membered heterocycloalkyl, wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl.

In certain of the foregoing embodiments, each R⁶ is independently selected from the group consisting of: C₁-C₆ alkyl and C₃-C₇ cycloalkyl.

As a non-limiting example of the foregoing embodiments, each R⁶ is isopropyl.

In certain embodiments (when B is 4-pyridyl), o=2 and p=2.

In certain of the foregoing embodiments, the substituted ring B is

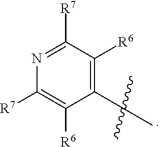

In certain of these embodiments, each R⁶ is independently selected from the group consisting of: C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO-C₁-C₆ alkyl; CONR⁸R⁹, and 4- to 6-membered heterocycloalkyl, wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C to aryl, NHCO(5- to 10-membered heteroaryl), NHCO(4- to 6-membered heterocycloalkyl), and NHCOC₂-C₆ alkynyl.

In certain of the foregoing embodiments, each R⁶ is independently selected from the group consisting of: C₁-C₆ alkyl, C₃-C₇ cycloalkyl, halo, and C₆-C₁₀ aryl.

As a non-limiting example of the foregoing embodiments, each R⁶ is independently selected from the group consisting of: methyl, isopropyl, cyclopropyl, fluoro, and phenyl.

In certain embodiments (when the substituted ring B is

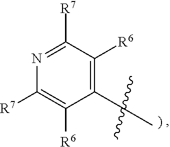

each R⁷ is independently selected from the group consisting of: C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO-C₁-C₆ alkyl; CONR⁸R⁹, and 4- to 6-membered heterocycloalkyl, wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl.

In certain of the foregoing embodiments, each R⁷ is independently selected from the group consisting of: C₁-C₆ alkyl, C₃-C₇ cycloalkyl, halo, and C₆-C₁₀ aryl.

As a non-limiting example of the foregoing embodiments, each R⁷ is independently selected from the group consisting of: methyl, isopropyl, cyclopropyl, fluoro, and phenyl.

In some embodiments (when the substituted ring B is

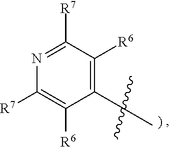

one pair of R⁶ and R⁷ on adjacent atoms, taken together with the atoms connecting them, independently form at least one C₄-C₈ carbocyclic ring or at least one 5- to 8-membered heterocyclic ring containing 1 or 2 heteroatoms and/or heteroatomic groups independently selected from O, NH, NR¹³, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹.

In certain of these embodiments, one pair of R⁶ and R⁷ on adjacent atoms, taken together with the atoms connecting them, independently form at least one C₄-C₈ carbocyclic ring, wherein the carbocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹.

In certain embodiments (when the substituted ring B is

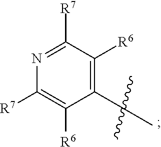

and one pair of R⁶ and R⁷ on adjacent atoms, taken together with the atoms connecting them, independently form at least one C₄-C₈ carbocyclic ring or at least one 5- to 8-membered heterocyclic ring containing 1 or 2 heteroatoms and/or heteroatomic groups independently selected from O, NH, NR¹³, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹), the C₄-C₅ carbocyclic ring is a C₅ carbocyclic ring optionally substituted with one or more oxo, CH₃, or hydroxy.

In certain of these embodiments, the C₅ carbocyclic ring is substituted with one CH₃.

In certain embodiments, the C₅ carbocyclic ring is geminally substituted with two CH₃.

In certain embodiments, the C₄-C₈ carbocyclic ring is a C₇ carbocyclic ring, wherein the C₇ carbocyclic ring is a bicyclic spirocycle, wherein the bicyclic spirocycle comprises a 5-membered ring and a 3-membered ring.

In some embodiments, the substituted ring B is

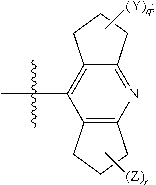

q is 0, 1, or 2; r is 0, 1, or 2; wherein each of Y and Z is independently selected from C₁-C₆ alkyl (e.g., methyl) and hydroxy; or wherein when two Y are attached to the same carbon, the two Y are taken together with the carbon they are attached to to form a cyclopropyl ring; or wherein when two Z are attached to the same carbon, the two Z are taken together with the carbon they are attached to form a cyclopropyl ring.

In some embodiments, the substituted ring B is

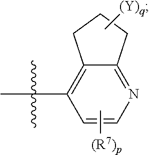

R⁷ is selected from C₁-C₆ alkyl (e.g., methyl, ethyl, or isopropyl), C₆-C₁₀ aryl (e.g., phenyl), and C₃-C₁₀ cycloalkyl (e.g., cyclopropyl); p is 0, 1, or 2; q is 0, 1, or 2; wherein each Y is independently selected from C₁-C₆ alkyl (e.g., methyl) and hydroxy; or when two Y are attached to the same carbon, the two Y are taken together with the carbon they are attached to to form a cyclopropyl ring.

In some embodiments, the substituted ring B is

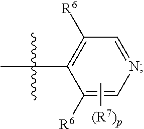

each R⁶ is independently selected from C₁-C₆ alkyl (e.g., isopropyl); each R⁷ is independently selected from halo (e.g., fluoro); p is 0 or 1.

In some embodiments, the substituted ring B is

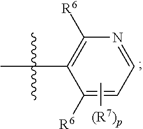

each R⁶ is independently selected from C₁-C₆ alkyl (e.g., isopropyl); each R⁷ is independently selected from halo (e.g., fluoro); p is 0 or 1.

In some embodiments, the substituted ring B is

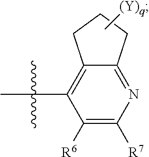

R⁶ is selected from C₁-C₆ alkyl (e.g., methyl, ethyl, or isopropyl) and C₃-C₁₀ cycloalkyl (e.g., cyclopropyl); R⁷ is selected from C₁-C₆ alkyl (e.g., methyl, ethyl, or isopropyl), C₁-C₆ haloalkyl (e.g., trifluoromethyl) and C₃-C₁₀ cycloalkyl (e.g., cyclopropyl or cyclobutyl); or R⁶ and R⁷, taken together with the atoms connecting them, independently form a C₅ carbocyclic ring optionally substituted with one or more C₁-C₆ alkyl (e.g., methyl); q is 0, 1, or 2; each Y is independently selected from C₁-C₆ alkyl (e.g., methyl); or when two Y are attached to the same carbon, the two Y are taken together with the carbon they are attached to to form a cyclopropyl ring.

The Group R³

In some embodiments, R³ is cyano.

In some embodiments, R³ is hydroxy.

In some embodiments, R³ is C₁-C₆ alkoxy.

In some embodiments, R³ is C(O)OR′.

In some embodiments, R³ is C(O)R′. In certain of these embodiments, R³ is C(O)H. In certain other embodiments, R³ is C(O)(C₁-C₆ alkyl).

In some embodiments, R³ is C(O)NHR¹⁴.

In some embodiments, R³ is C(O)NR′R″.

In some embodiments, R³ is S(O)₀₋₂R′ (e.g., S(O)₂R′).

In some embodiments, R³ is S(O)₂NR′R″ (e.g., S(O)₂R′R″).

In some embodiments, R³ is R¹⁴—(C₀-C₂ alkylene)- wherein the C₀-C₂ alkylene is optionally substituted with oxo. In certain of these embodiments, R³ is C(O)R¹⁴.

The Group R¹⁴

In some embodiments, R¹⁴ is 5- to 10-membered monocyclic or bicyclic heteroaryl optionally independently substituted with 1 or 2 R⁶.

In some embodiments, R¹⁴ is C₆-C₁₀ monocyclic or bicyclic aryl optionally independently substituted with 1 or 2 R⁶.

The Variable R′ and R″

In some embodiments, each of R′ and R″ is independently H or C₁-C₃ alkyl.

In some embodiments, each of R′ and R″ is H.

In some embodiments, R′ is H.

In some embodiments, R′ is C₁-C₆ alkyl.

In some embodiments, R′ is H; and R″ is other than H.

The Moiety S(═O)(NHR³)═N—

In some embodiments, the sulfur in the moiety S(═O)(NHR³)═N— has (S) stereochemistry.

In some embodiments, the sulfur in the moiety S(═O)(NHR³)═N— has (R) stereochemistry.

The Group R¹⁰

In some embodiments, R¹⁰ is C₁-C₆ alkyl.

In some embodiments, R¹⁰ is methyl.

In some embodiments, R¹⁰ is ethyl.

The Groups R⁸ and R⁹

In some embodiments, each of R⁸ and R⁹ at each occurrence is independently selected from hydrogen, C₁-C₆ alkyl, (C═NR¹³)NR¹¹R¹², S(O₂)C₁-C₆ alkyl, S(O₂)NR¹¹R¹², COR¹³, CO₂R¹³ and CONR¹¹R¹²; wherein the C₁-C₆ alkyl is optionally substituted with one or more hydroxy, halo, C₁-C₆ alkoxy, C₆-C₁₀ aryl, 5- to 10-membered heteroaryl, C₃-C₇ cycloalkyl or 3- to 7-membered heterocycloalkyl; or R⁸ and R⁹ taken together with the nitrogen they are attached to form a 3- to 7-membered ring optionally containing one or more heteroatoms in addition to the nitrogen they are attached to.

In some embodiments, each of R⁸ and R⁹ at each occurrence is independently selected from hydrogen, C₁-C₆ alkyl, (C═NR¹³)NR¹¹R¹², S(O₂)C₁-C₆ alkyl, S(O₂)NR¹¹R¹², COR¹³, CO₂R¹³ and CONR¹¹R¹²; wherein the C₁-C₆ alkyl is optionally substituted with one or more hydroxy, halo, C₁-C₆ alkoxy, C₆-C₁₀ aryl, 5- to 10-membered heteroaryl, C₃-C₇ cycloalkyl or 3- to 7-membered heterocycloalkyl; or R⁸ and R⁹ taken together with the nitrogen they are attached to form a 3- to 7-membered ring optionally containing one or more heteroatoms in addition to the nitrogen they are attached to.

In some embodiments, each of R⁸ and R⁹ at each occurrence is hydrogen,

In some embodiments, each R⁸ at each occurrence is hydrogen and each R⁹ at each occurrence is C₁-C₆ alkyl.

In some embodiments, each R⁸ at each occurrence is hydrogen and each R⁹ at each occurrence is methyl.

In some embodiments, each R⁸ at each occurrence is hydrogen and each R⁹ at each occurrence is ethyl.

In some embodiments, each of R⁸ and R⁹ at each occurrence is methyl.

In some embodiments, each of R⁸ and R⁹ at each occurrence is ethyl.

In some embodiments, R⁸ and R⁹ taken together with the nitrogen they are attached to form a 3-membered ring.

In some embodiments, R⁸ and R⁹ taken together with the nitrogen they are attached to form a 4-membered ring.

In some embodiments, R⁸ and R⁹ taken together with the nitrogen they are attached to form a 5-membered ring.

In some embodiments, R⁸ and R⁹ taken together with the nitrogen they are attached to form a 6-membered ring optionally containing one or more oxygen atoms in addition to the nitrogen they are attached to.

In some embodiments, R⁸ and R⁹ taken together with the nitrogen they are attached to form a 6-membered ring optionally containing one or more nitrogen atoms in addition to the nitrogen they are attached to.

In some embodiments, R⁸ and R⁹ taken together with the nitrogen they are attached to form a 7-membered ring.

In some embodiments, one of R⁸ and R⁹ is C(O)R¹³; R¹³ is —(Z¹-Z²)ₐ₁-Z³; and a1 is 0.

In certain of these embodiments, the other one of R⁸ and R⁹ is hydrogen.

As a non-limiting example of the foregoing embodiments, NR⁸R⁹ is selected from the group consisting of: NHCOC₆-C₁₀ aryl, NHCO(5- to 10-membered heteroaryl), NHCO(3- to 7-membered heterocycloalkyl), and NHCOC₂-C₆ alkynyl.

In some embodiments, one of R⁸ and R⁹ is C(O)R¹³; R¹³ is C₁-C₆ alkyl.

In certain embodiments, NR⁸R⁹ is selected from the group consisting of: NH₂, NHC₁-C₆ alkyl, N(C₁-C₆ alkyl)₂, NHCOC₁-C₆ alkyl, NHCOC₆-C₁₀ aryl, NHCO(5- to 10-membered heteroaryl), NHCO(3- to 7-membered heterocycloalkyl), NHCOC₂-C₆ alkynyl, and NHCOOC₁-C₆ alkyl.

The Group R¹³

In some embodiments, R¹³ is C₁-C₆ alkyl.

In some embodiments, R¹³ is methyl.

In some embodiments, R¹³ is ethyl.

In some embodiments, R¹³ is —(Z¹-Z²)ₐ₁-Z³.

In certain of these embodiments, a1 is 0. In certain embodiments, Z³ is C₆-C₁₀ aryl or 5- to 10-membered heteroaryl.

In some embodiments, R¹³ is C₆-C₁₀ aryl.

In some embodiments, R¹³ is phenyl.

In some embodiments, R¹³ is 5- to 10-membered heteroaryl.

The Groups R¹¹ and R¹²

In some embodiments, each of R¹¹ and R¹² at each occurrence is independently selected from hydrogen and C₁-C₆ alkyl.

In some embodiments, each of R¹¹ and R¹² at each occurrence is hydrogen,

In some embodiments, each R¹¹ at each occurrence is hydrogen and each R¹² at each occurrence is C₁-C₆ alkyl.

In some embodiments, each R¹¹ at each occurrence is hydrogen and each R¹² at each occurrence is methyl.

In some embodiments, each R¹¹ at each occurrence is hydrogen and each R¹² at each occurrence is ethyl.

In some embodiments, each of R¹¹ and R¹² at each occurrence is methyl.

In some embodiments, each of R¹¹ and R¹² at each occurrence is ethyl.

The Group R¹⁵

In some embodiments, R¹⁵ is —(Z⁴-Z⁵)ₐ₂-Z⁶.

In certain embodiments, a2 is 1-5.

In certain embodiments, the Z⁴ group directly attached to R¹ or R² is —O—.

In certain embodiments, each Z⁴ is independently —O— or —NH—, provided that the Z⁴ group directly attached to R¹ or R² is —O—.

In certain embodiments, each Z⁴ is —O—.

In certain embodiments, each Z⁵ is independently C₂-C₆ alkylene optionally substituted with one or more substituents independently selected from oxo, halo, and hydroxyl. In certain these embodiments, each Z⁵ is independently C₂-C₄ (e.g., C₂-C₃ (e.g., C₂ or C₃)) alkylene.

In certain embodiments, Z⁶ is OH.

In certain embodiments, Z⁶ is NHC(O)(C₁-C₆ alkoxy).

In certain embodiments, Z⁶ is C₆-C₁₀ aryl.

In certain embodiments, Z⁶ is C₁-C₆ alkoxy.

In certain embodiments of R¹⁵, a2=1; and Z⁴ is 0. In certain of these embodiments, Z⁵ is C₂-C₄ (e.g., C₂-C₃ (e.g., C₂ or C₃)) alkylene. In certain of the foregoing embodiments, Z⁶ is selected from OH, NHC(O)(C1-C6 alkoxy), and C₁-C₆ alkoxy.

As non-limiting examples, R¹⁵ is selected from:

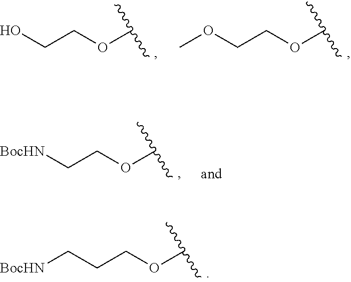

In certain embodiments of R¹⁵, a2=1; and each Z⁴ is O. In certain of these embodiments, Z⁵ is C₂-C₄ (e.g., C₂-C₃ (e.g., C₂ or C₃)) alkylene. In certain of the foregoing embodiments, Z⁶ is selected from OH, NHC(O)(C1-C6 alkoxy), and C₁-C₆ alkoxy. In certain other of the foregoing embodiments, Z⁶ is C₆-C₁₀ aryl (e.g., R¹⁵ is

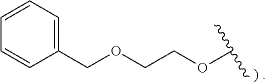 [SMILES: *OCCOCc1ccccc1]

In certain embodiments of R¹⁵, a2≥2 (e.g., a2 is 3 or 4); each Z⁴ is 0; and each Z⁵ is ethylene. In certain of these embodiments Z⁶ is OH. In certain other embodiments, Z⁶ is NHC(O)(C₁-C₆ alkoxy) (e.g., Boc). As a non-limiting example, R¹⁵ is:

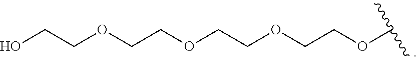 [SMILES: *OCCOCCOCCOCCO]

The Group L^{A}

In some embodiments, L^{A} is a bond.

In some embodiments, L^{A} is CH₂.

In some embodiments, L^{A} is C₂-C₄ alkylene.

In some embodiments of the compound of formula AA,

the substituted ring A is

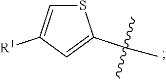

and R¹ is selected from:

C₁-C₆ alkyl optionally substituted with one or more hydroxy; C₃-C₇ cycloalkyl optionally substituted with one or more hydroxy; 3- to 7-membered heterocycloalkyl optionally substituted with one or more hydroxy; C₁-C₆ alkyl substituted with one or more oxo; C₃-C₇ cycloalkyl substituted with one or more oxo; C₁-C₆ alkyl substituted with one or more C₁-C₆ alkoxy; C₃-C₇ cycloalkyl substituted with one or more C₁-C₆ alkoxy; C₁-C₆ haloalkyl; C₁-C₆ alkoxy; C₁-C₆ haloalkoxy; halo; CN; NO₂; COC₁-C₆ alkyl; CO-C₆-C₁₀ aryl; CO(5- to 10-membered heteroaryl); CO₂C₁-C₆ alkyl; CO₂C₃-C₈ cycloalkyl; OCOC₁-C₆ alkyl; OCOC₆-C₁₀ aryl; OCO(5- to 10-membered heteroaryl); OCO(3- to 7-membered heterocycloalkyl); C₆-C₁₀ aryl; 5- to 10-membered heteroaryl; NH₂; NHC₁-C₆ alkyl; N(C₁-C₆ alkyl)₂; CONR⁸R⁹; SF₅; and S(O₂)C₁-C₆ alkyl.

In some embodiments of the compound of formula AA,

the substituted ring A is

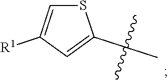

and R¹ is selected from:
1-hydroxy-2-methylpropan-2-yl; methyl; isopropyl; 2-hydroxy-2-propyl; hydroxymethyl; 1-hydroxyethyl; 2-hydroxyethyl; 1-hydroxy-2-propyl; 1-hydroxy-1-cyclopropyl; 1-hydroxy-1-cyclobutyl; 1-hydroxy-1-cyclopentyl; 1-hydroxy-1-cyclohexyl; morpholinyl; 1,3-dioxolan-2-yl; COCH₃; COCH₂CH₃; 2-methoxy-2-propyl; (dimethylamino)methyl; 1-(dimethylamino)ethyl; fluoro; chloro; phenyl; pyridyl; pyrazolyl; and S(O₂)CH₃.

In some embodiments of the compound of formula AA,

the substituted ring A is

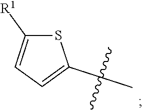

and R¹ is selected from:
C₁-C₆ alkyl optionally substituted with one or more hydroxy; C₃-C₇ cycloalkyl optionally substituted with one or more hydroxy; 3- to 7-membered heterocycloalkyl optionally substituted with one or more hydroxy; C₁-C₆ alkyl substituted with one or more oxo; C₃-C₇ cycloalkyl substituted with one or more oxo; C₁-C₆ alkyl substituted with one or more C₁-C₆ alkoxy; C₃-C₇ cycloalkyl substituted with one or more C₁-C₆ alkoxy; C₁-C₆ alkyl substituted with one or more NR⁸R⁹; 3- to 7-membered heterocycloalkyl substituted with one or more NR⁸R⁹; C₁-C₆ haloalkyl; C₁-C₆ alkoxy; C₁-C₆ haloalkoxy; halo; CN; NO₂; COC₁-C₆ alkyl; CO-C₆-C₁₀ aryl; CO(5- to 10-membered heteroaryl); CO₂C₁-C₆ alkyl; CO₂C₃-C₈ cycloalkyl; OCOC₁-C₆ alkyl; OCOC₆-C₁₀ aryl; OCO(5- to 10-membered heteroaryl); OCO(3- to 7-membered heterocycloalkyl); C₆-C₁₀ aryl; 5- to 10-membered heteroaryl; NH₂; NHC₁-C₆ alkyl; N(C₁-C₆ alkyl)₂; CONR⁸R⁹; SF₅; and S(O₂)C₁-C₆ alkyl.

In some embodiments of the compound of formula AA,

the substituted ring A is

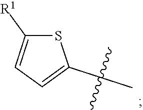

and R¹ is selected from:
1-hydroxy-2-methylpropan-2-yl; methyl; isopropyl; 2-hydroxy-2-propyl; hydroxymethyl; 1-hydroxyethyl; 2-hydroxyethyl; 1-hydroxy-2-propyl; 1-hydroxy-1-cyclopropyl; 1-hydroxy-1-cyclobutyl; 1-hydroxy-1-cyclopentyl; 1-hydroxy-1-cyclohexyl; morpholinyl; 1,3-dioxolan-2-yl; COCH₃; COCH₂CH₃; 2-methoxy-2-propyl; (dimethylamino)methyl; 1-(dimethylamino)ethyl; fluoro; chloro; phenyl; pyridyl; pyrazolyl; and S(O₂)CH₃.

In some embodiments of the compound of formula AA,

the substituted ring A is

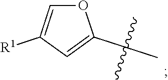

and R¹ is selected from:
C₁-C₆ alkyl optionally substituted with one or more hydroxy; C₃-C₇ cycloalkyl optionally substituted with one or more hydroxy; 3- to 7-membered heterocycloalkyl optionally substituted with one or more hydroxy; C₁-C₆ alkyl substituted with one or more oxo; C₃-C₇ cycloalkyl substituted with one or more oxo; C₁-C₆ alkyl substituted with one or more C₁-C₆ alkoxy; C₃-C₇ cycloalkyl substituted with one or more C₁-C₆ alkoxy; C₁-C₆ alkyl substituted with one or more NR⁸R⁹; 3- to 7-membered heterocycloalkyl substituted with one or more NR⁸R⁹; C₁-C₆ haloalkyl; C₁-C₆ alkoxy; C₁-C₆ haloalkoxy; halo; CN; NO₂; COC₁-C₆ alkyl; CO-C₆-C₁₀ aryl; CO(5- to 10-membered heteroaryl); CO₂C₁-C₆ alkyl; CO₂C₃-C₈ cycloalkyl; OCOC₁-C₆ alkyl; OCOC₆-C₁₀ aryl; OCO(5- to 10-membered heteroaryl); OCO(3- to 7-membered heterocycloalkyl); C₆-C₁₀ aryl; 5- to 10-membered heteroaryl; NH₂; NHC₁-C₆ alkyl; N(C₁-C₆ alkyl)₂; CONR⁸R⁹; SF₅; and S(O₂)C₁-C₆ alkyl.

In some embodiments of the compound of formula AA,

the substituted ring A is

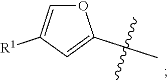

and R¹ is selected from:
1-hydroxy-2-methylpropan-2-yl; methyl; isopropyl; 2-hydroxy-2-propyl; hydroxymethyl; 1-hydroxyethyl; 2-hydroxyethyl; 1-hydroxy-2-propyl; 1-hydroxy-1-cyclopropyl; 1-hydroxy-1-cyclobutyl; 1-hydroxy-1-cyclopentyl; 1-hydroxy-1-cyclohexyl; morpholinyl; 1,3-dioxolan-2-yl; COCH₃; COCH₂CH₃; 2-methoxy-2-propyl; (dimethylamino)methyl; 1-(dimethylamino)ethyl; fluoro; chloro; phenyl; pyridyl; pyrazolyl; and S(O₂)CH₃.

In some embodiments of the compound of formula AA,

the substituted ring A is

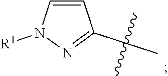

and R¹ is selected from:
C₁-C₆ alkyl optionally substituted with one or more hydroxy; C₃-C₇ cycloalkyl optionally substituted with one or more hydroxy; 3- to 7-membered heterocycloalkyl optionally substituted with one or more hydroxy; C₁-C₆ alkyl substituted with one or more oxo; C₃-C₇ cycloalkyl substituted with one or more oxo; C₁-C₆ alkyl substituted with one or more C₁-C₆ alkoxy; C₃-C₇ cycloalkyl substituted with one or more C₁-C₆ alkoxy; C₁-C₆ alkyl substituted with one or more NR⁸R⁹; 3- to 7-membered heterocycloalkyl substituted with one or more NR⁸R⁹; C₁-C₆ haloalkyl; C₁-C₆ alkoxy; C₁-C₆ haloalkoxy; halo; CN; NO₂; COC₁-C₆ alkyl; CO-C₆-C₁₀ aryl; CO(5- to 10-membered heteroaryl); CO₂C₁-C₆ alkyl; CO₂C₃-C₈ cycloalkyl; OCOC₁-C₆ alkyl; OCOC₆-C₁₀ aryl; OCO(5- to 10-membered heteroaryl); OCO(3- to 7-membered heterocycloalkyl); C₆-C₁₀ aryl; 5- to 10-membered heteroaryl; NH₂; NHC₁-C₆ alkyl; N(C₁-C₆ alkyl)₂; CONR⁸R⁹; SF₅; and S(O₂)C₁-C₆ alkyl.

In some embodiments of the compound of formula AA,

the substituted ring A is

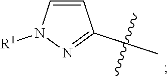

and R¹ is selected from:
1-hydroxy-2-methylpropan-2-yl; methyl; isopropyl; 2-hydroxy-2-propyl; hydroxymethyl; 1-hydroxyethyl; 2-hydroxyethyl; 1-hydroxy-2-propyl; 1-hydroxy-1-cyclopropyl; 1-hydroxy-1-cyclobutyl; 1-hydroxy-1-cyclopentyl; 1-hydroxy-1-cyclohexyl; morpholinyl; 1,3-dioxolan-2-yl; COCH₃; COCH₂CH₃; 2-methoxy-2-propyl; (dimethylamino)methyl; 1-(dimethylamino)ethyl; fluoro; chloro; phenyl; pyridyl; pyrazolyl; and S(O₂)CH₃.

In some embodiments of the compound of formula AA,

the substituted ring A is

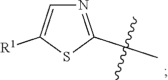

and R¹ is selected from:
C₁-C₆ alkyl optionally substituted with one or more hydroxy; C₃-C₇ cycloalkyl optionally substituted with one or more hydroxy; 3- to 7-membered heterocycloalkyl optionally substituted with one or more hydroxy; C₁-C₆ alkyl substituted with one or more oxo; C₃-C₇ cycloalkyl substituted with one or more oxo; C₁-C₆ alkyl substituted with one or more C₁-C₆ alkoxy; C₃-C₇ cycloalkyl substituted with one or more C₁-C₆ alkoxy; C₁-C₆ alkyl substituted with one or more NR⁸R⁹; 3- to 7-membered heterocycloalkyl substituted with one or more NR⁸R⁹; C₁-C₆ haloalkyl; C₁-C₆ alkoxy; C₁-C₆ haloalkoxy; halo; CN; NO₂; COC₁-C₆ alkyl; CO-C₆-C₁₀ aryl; CO(5- to 10-membered heteroaryl); CO₂C₁-C₆ alkyl; CO₂C₃-C₈ cycloalkyl; OCOC₁-C₆ alkyl; OCOC₆-C₁₀ aryl; OCO(5- to 10-membered heteroaryl); OCO(3- to 7-membered heterocycloalkyl); C₆-C₁₀ aryl; 5- to 10-membered heteroaryl; NH₂; NHC₁-C₆ alkyl; N(C₁-C₆ alkyl)₂; CONR⁸R⁹; SF₅; and S(O₂)C₁-C₆ alkyl.

In some embodiments of the compound of formula AA,

the substituted ring A is

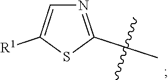

and R¹ is selected from:
1-hydroxy-2-methylpropan-2-yl; methyl; isopropyl; 2-hydroxy-2-propyl; hydroxymethyl; 1-hydroxyethyl; 2-hydroxyethyl; 1-hydroxy-2-propyl; 1-hydroxy-1-cyclopropyl; 1-hydroxy-1-cyclobutyl; 1-hydroxy-1-cyclopentyl; 1-hydroxy-1-cyclohexyl; morpholinyl; 1,3-dioxolan-2-yl; COCH₃; COCH₂CH₃; 2-methoxy-2-propyl; (dimethylamino)methyl; 1-(dimethylamino)ethyl; fluoro; chloro; phenyl; pyridyl; pyrazolyl; and S(O₂)CH₃.

In some embodiments of the compound of formula AA,

the substituted ring A is

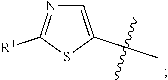

and R¹ is selected from:
C₁-C₆ alkyl optionally substituted with one or more hydroxy; C₃-C₇ cycloalkyl optionally substituted with one or more hydroxy; 3- to 7-membered heterocycloalkyl optionally substituted with one or more hydroxy; C₁-C₆ alkyl substituted with one or more oxo; C₃-C₇ cycloalkyl substituted with one or more oxo; C₁-C₆ alkyl substituted with one or more C₁-C₆ alkoxy; C₃-C₇ cycloalkyl substituted with one or more C₁-C₆ alkoxy; C₁-C₆ alkyl substituted with one or more NR⁸R⁹; 3- to 7-membered heterocycloalkyl substituted with one or more NR⁸R⁹; C₁-C₆ haloalkyl; C₁-C₆ alkoxy; C₁-C₆ haloalkoxy; halo; CN; NO₂; COC₁-C₆ alkyl; CO-C₆-C₁₀ aryl; CO(5- to 10-membered heteroaryl); CO₂C₁-C₆ alkyl; CO₂C₃-C₈ cycloalkyl; OCOC₁-C₆ alkyl; OCOC₆-C₁₀ aryl; OCO(5- to 10-membered heteroaryl); OCO(3- to 7-membered heterocycloalkyl); C₆-C₁₀ aryl; 5- to 10-membered heteroaryl; NH₂; NHC₁-C₆ alkyl; N(C₁-C₆ alkyl)₂; CONR⁸R⁹; SF₅; and S(O₂)C₁-C₆ alkyl.

In some embodiments of the compound of formula AA,

the substituted ring A is

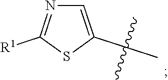

and R¹ is selected from:
1-hydroxy-2-methylpropan-2-yl; methyl; isopropyl; 2-hydroxy-2-propyl; hydroxymethyl; 1-hydroxyethyl; 2-hydroxyethyl; 1-hydroxy-2-propyl; 1-hydroxy-1-cyclopropyl; 1-hydroxy-1-cyclobutyl; 1-hydroxy-1-cyclopentyl; 1-hydroxy-1-cyclohexyl; morpholinyl; 1,3-dioxolan-2-yl; COCH₃; COCH₂CH₃; 2-methoxy-2-propyl; (dimethylamino)methyl; 1-(dimethylamino)ethyl; fluoro; chloro; phenyl; pyridyl; pyrazolyl; and S(O₂)CH₃.

In some embodiments of the compound of formula AA,

the substituted ring A is

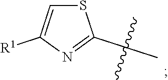

and R¹ is selected from:
C₁-C₆ alkyl optionally substituted with one or more hydroxy; C₃-C₇ cycloalkyl optionally substituted with one or more hydroxy; 3- to 7-membered heterocycloalkyl optionally substituted with one or more hydroxy; C₁-C₆ alkyl substituted with one or more oxo; C₃-C₇ cycloalkyl substituted with one or more oxo; C₁-C₆ alkyl substituted with one or more C₁-C₆ alkoxy; C₃-C₇ cycloalkyl substituted with one or more C₁-C₆ alkoxy; C₁-C₆ alkyl substituted with one or more NR⁸R⁹; 3- to 7-membered heterocycloalkyl substituted with one or more NR⁸R⁹; C₁-C₆ haloalkyl; C₁-C₆ alkoxy; C₁-C₆ haloalkoxy; halo; CN; NO₂; COC₁-C₆ alkyl; CO-C₆-C₁₀ aryl; CO(5- to 10-membered heteroaryl); CO₂C₁-C₆ alkyl; CO₂C₃-C₈ cycloalkyl; OCOC₁-C₆ alkyl; OCOC₆-C₁₀ aryl; OCO(5- to 10-membered heteroaryl); OCO(3- to 7-membered heterocycloalkyl); C₆-C₁₀ aryl; 5- to 10-membered heteroaryl; NH₂; NHC₁-C₆ alkyl; N(C₁-C₆ alkyl)₂; CONR⁸R⁹; SF₅; and S(O₂)C₁-C₆ alkyl.

In some embodiments of the compound of formula AA,

the substituted ring A is

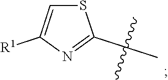

and R¹ is selected from:
1-hydroxy-2-methylpropan-2-yl; methyl; isopropyl; 2-hydroxy-2-propyl; hydroxymethyl; 1-hydroxyethyl; 2-hydroxyethyl; 1-hydroxy-2-propyl; 1-hydroxy-1-cyclopropyl; 1-hydroxy-1-cyclobutyl; 1-hydroxy-1-cyclopentyl; 1-hydroxy-1-cyclohexyl; morpholinyl; 1,3-dioxolan-2-yl; COCH₃; COCH₂CH₃; 2-methoxy-2-propyl; (dimethylamino)methyl; 1-(dimethylamino)ethyl; fluoro; chloro; phenyl; pyridyl; pyrazolyl; and S(O₂)CH₃.

In some embodiments of the compound of formula AA,

the substituted ring A is

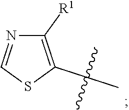

and R¹ is selected from:
C₁-C₆ alkyl optionally substituted with one or more hydroxy; C₃-C₇ cycloalkyl optionally substituted with one or more hydroxy; 3- to 7-membered heterocycloalkyl optionally substituted with one or more hydroxy; C₁-C₆ alkyl substituted with one or more oxo; C₃-C₇ cycloalkyl substituted with one or more oxo; C₁-C₆ alkyl substituted with one or more C₁-C₆ alkoxy; C₃-C₇ cycloalkyl substituted with one or more C₁-C₆ alkoxy; C₁-C₆ alkyl substituted with one or more NR⁸R⁹; 3- to 7-membered heterocycloalkyl substituted with one or more NR⁸R⁹; C₁-C₆ haloalkyl; C₁-C₆ alkoxy; C₁-C₆ haloalkoxy; halo; CN; NO₂; COC₁-C₆ alkyl; CO-C₆-C₁₀ aryl; CO(5- to 10-membered heteroaryl); CO₂C₁-C₆ alkyl; CO₂C₃-C₈ cycloalkyl; OCOC₁-C₆ alkyl; OCOC₆-C₁₀ aryl; OCO(5- to 10-membered heteroaryl); OCO(3- to 7-membered heterocycloalkyl); C₆-C₁₀ aryl; 5- to 10-membered heteroaryl; NH₂; NHC₁-C₆ alkyl; N(C₁-C₆ alkyl)₂; CONR⁸R⁹; SF₅; and S(O₂)C₁-C₆ alkyl.

In some embodiments of the compound of formula AA,

the substituted ring A is

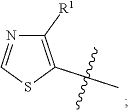

and R¹ is selected from:
1-hydroxy-2-methylpropan-2-yl; methyl; isopropyl; 2-hydroxy-2-propyl; hydroxymethyl; 1-hydroxyethyl; 2-hydroxyethyl; 1-hydroxy-2-propyl; 1-hydroxy-1-cyclopropyl; 1-hydroxy-1-cyclobutyl; 1-hydroxy-1-cyclopentyl; 1-hydroxy-1-cyclohexyl; morpholinyl; 1,3-dioxolan-2-yl; COCH₃; COCH₂CH₃; 2-methoxy-2-propyl; (dimethylamino)methyl; 1-(dimethylamino)ethyl; fluoro; chloro; phenyl; pyridyl; pyrazolyl; and S(O₂)CH₃.

In some embodiments of the compound of formula AA,

the substituted ring A is

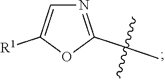

and R¹ is selected from:
C₁-C₆ alkyl optionally substituted with one or more hydroxy; C₃-C₇ cycloalkyl optionally substituted with one or more hydroxy; 3- to 7-membered heterocycloalkyl optionally substituted with one or more hydroxy; C₁-C₆ alkyl substituted with one or more oxo; C₃-C₇ cycloalkyl substituted with one or more oxo; C₁-C₆ alkyl substituted with one or more C₁-C₆ alkoxy; C₃-C₇ cycloalkyl substituted with one or more C₁-C₆ alkoxy; C₁-C₆ alkyl substituted with one or more NR⁸R⁹; 3- to 7-membered heterocycloalkyl substituted with one or more NR⁸R⁹; C₁-C₆ haloalkyl; C₁-C₆ alkoxy; C₁-C₆ haloalkoxy; halo; CN; NO₂; COC₁-C₆ alkyl; CO-C₆-C₁₀ aryl; CO(5- to 10-membered heteroaryl); CO₂C₁-C₆ alkyl; CO₂C₃-C₈ cycloalkyl; OCOC₁-C₆ alkyl; OCOC₆-C₁₀ aryl; OCO(5- to 10-membered heteroaryl); OCO(3- to 7-membered heterocycloalkyl); C₆-C₁₀ aryl; 5- to 10-membered heteroaryl; NH₂; NHC₁-C₆ alkyl; N(C₁-C₆ alkyl)₂; CONR⁸R⁹; SF₅; and S(O₂)C₁-C₆ alkyl.

In some embodiments of the compound of formula AA,

the substituted ring A is

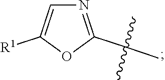

and R¹ is selected from:
1-hydroxy-2-methylpropan-2-yl; methyl; isopropyl; 2-hydroxy-2-propyl; hydroxymethyl; 1-hydroxyethyl; 2-hydroxyethyl; 1-hydroxy-2-propyl; 1-hydroxy-1-cyclopropyl; 1-hydroxy-1-cyclobutyl; 1-hydroxy-1-cyclopentyl; 1-hydroxy-1-cyclohexyl; morpholinyl; 1,3-dioxolan-2-yl; COCH₃; COCH₂CH₃; 2-methoxy-2-propyl; (dimethylamino)methyl; 1-(dimethylamino)ethyl; fluoro; chloro; phenyl; pyridyl; pyrazolyl; and S(O₂)CH₃.

In some embodiments of the compound of formula AA,

the substituted ring A is

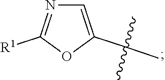

and R¹ is selected from:
C₁-C₆ alkyl optionally substituted with one or more hydroxy; C₃-C₇ cycloalkyl optionally substituted with one or more hydroxy; 3- to 7-membered heterocycloalkyl optionally substituted with one or more hydroxy; C₁-C₆ alkyl substituted with one or more oxo; C₃-C₇ cycloalkyl substituted with one or more oxo; C₁-C₆ alkyl substituted with one or more C₁-C₆ alkoxy; C₃-C₇ cycloalkyl substituted with one or more C₁-C₆ alkoxy; C₁-C₆ alkyl substituted with one or more NR⁸R⁹; 3- to 7-membered heterocycloalkyl substituted with one or more NR⁸R⁹; C₁-C₆ haloalkyl; C₁-C₆ alkoxy; C₁-C₆ haloalkoxy; halo; CN; NO₂; COC₁-C₆ alkyl; CO-C₆-C₁₀ aryl; CO(5- to 10-membered heteroaryl); CO₂C₁-C₆ alkyl; CO₂C₃-C₈ cycloalkyl; OCOC₁-C₆ alkyl; OCOC₆-C₁₀ aryl; OCO(5- to 10-membered heteroaryl); OCO(3- to 7-membered heterocycloalkyl); C₆-C₁₀ aryl; 5- to 10-membered heteroaryl; NH₂; NHC₁-C₆ alkyl; N(C₁-C₆ alkyl)₂; CONR⁸R⁹; SF₅; and S(O₂)C₁-C₆ alkyl.

In some embodiments of the compound of formula AA,

the substituted ring A is

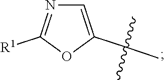

and R¹ is selected from:
1-hydroxy-2-methylpropan-2-yl; methyl; isopropyl; 2-hydroxy-2-propyl; hydroxymethyl; 1-hydroxyethyl; 2-hydroxyethyl; 1-hydroxy-2-propyl; 1-hydroxy-1-cyclopropyl; 1-hydroxy-1-cyclobutyl; 1-hydroxy-1-cyclopentyl; 1-hydroxy-1-cyclohexyl; morpholinyl; 1,3-dioxolan-2-yl; COCH₃; COCH₂CH₃; 2-methoxy-2-propyl; (dimethylamino)methyl; 1-(dimethylamino)ethyl; fluoro; chloro; phenyl; pyridyl; pyrazolyl; and S(O₂)CH₃.

In some embodiments of the compound of formula AA,

the substituted ring A is

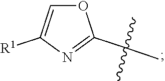

and R¹ is selected from:
C₁-C₆ alkyl optionally substituted with one or more hydroxy; C₃-C₇ cycloalkyl optionally substituted with one or more hydroxy; 3- to 7-membered heterocycloalkyl optionally substituted with one or more hydroxy; C₁-C₆ alkyl substituted with one or more oxo; C₃-C₇ cycloalkyl substituted with one or more oxo; C₁-C₆ alkyl substituted with one or more C₁-C₆ alkoxy; C₃-C₇ cycloalkyl substituted with one or more C₁-C₆ alkoxy; C₁-C₆ alkyl substituted with one or more NR⁸R⁹; 3- to 7-membered heterocycloalkyl substituted with one or more NR⁸R⁹; C₁-C₆ haloalkyl; C₁-C₆ alkoxy; C₁-C₆ haloalkoxy; halo; CN; NO₂; COC₁-C₆ alkyl; CO-C₆-C₁₀ aryl; CO(5- to 10-membered heteroaryl); CO₂C₁-C₆ alkyl; CO₂C₃-C₈ cycloalkyl; OCOC₁-C₆ alkyl; OCOC₆-C₁₀ aryl; OCO(5- to 10-membered heteroaryl); OCO(3- to 7-membered heterocycloalkyl); C₆-C₁₀ aryl; 5- to 10-membered heteroaryl; NH₂; NHC₁-C₆ alkyl; N(C₁-C₆ alkyl)₂; CONR⁸R⁹; SF₅; and S(O₂)C₁-C₆ alkyl.

In some embodiments of the compound of formula AA,

the substituted ring A is

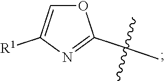

and R¹ is selected from:
1-hydroxy-2-methylpropan-2-yl; methyl; isopropyl; 2-hydroxy-2-propyl; hydroxymethyl; 1-hydroxyethyl; 2-hydroxyethyl; 1-hydroxy-2-propyl; 1-hydroxy-1-cyclopropyl; 1-hydroxy-1-cyclobutyl; 1-hydroxy-1-cyclopentyl; 1-hydroxy-1-cyclohexyl; morpholinyl; 1,3-dioxolan-2-yl; COCH₃; COCH₂CH₃; 2-methoxy-2-propyl; (dimethylamino)methyl; 1-(dimethylamino)ethyl; fluoro; chloro; phenyl; pyridyl; pyrazolyl; and S(O₂)CH₃.

In some embodiments of the compound of formula AA,

the substituted ring A is

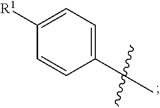

and R¹ is selected from:
C₁-C₆ alkyl optionally substituted with one or more hydroxy; C₃-C₇ cycloalkyl optionally substituted with one or more hydroxy; 3- to 7-membered heterocycloalkyl optionally substituted with one or more hydroxy; C₁-C₆ alkyl substituted with one or more oxo; C₃-C₇ cycloalkyl substituted with one or more oxo; C₁-C₆ alkyl substituted with one or more C₁-C₆ alkoxy; C₃-C₇ cycloalkyl substituted with one or more C₁-C₆ alkoxy; C₁-C₆ alkyl substituted with one or more NR⁸R⁹; 3- to 7-membered heterocycloalkyl substituted with one or more NR⁸R⁹; C₁-C₆ haloalkyl; C₁-C₆ alkoxy; C₁-C₆ haloalkoxy; halo; CN; NO₂; COC₁-C₆ alkyl; CO-C₆-C₁₀ aryl; CO(5- to 10-membered heteroaryl); CO₂C₁-C₆ alkyl; CO₂C₃-C₈ cycloalkyl; OCOC₁-C₆ alkyl; OCOC₆-C₁₀ aryl; OCO(5- to 10-membered heteroaryl); OCO(3- to 7-membered heterocycloalkyl); C₆-C₁₀ aryl; 5- to 10-membered heteroaryl; NH₂; NHC₁-C₆ alkyl; N(C₁-C₆ alkyl)₂; CONR⁸R⁹; SF₅; and S(O₂)C₁-C₆ alkyl.

In some embodiments of the compound of formula AA,

the substituted ring A is

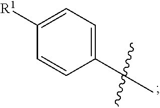

and R¹ is selected from:
1-hydroxy-2-methylpropan-2-yl; methyl; isopropyl; 2-hydroxy-2-propyl; hydroxymethyl; 1-hydroxyethyl; 2-hydroxyethyl; 1-hydroxy-2-propyl; 1-hydroxy-1-cyclopropyl; 1-hydroxy-1-cyclobutyl; 1-hydroxy-1-cyclopentyl; 1-hydroxy-1-cyclohexyl; morpholinyl; 1,3-dioxolan-2-yl; COCH₃; COCH₂CH₃; 2-methoxy-2-propyl; (dimethylamino)methyl; 1-(dimethylamino)ethyl; fluoro; chloro; phenyl; pyridyl; pyrazolyl; and S(O₂)CH₃.

In some embodiments of the compound of formula AA,

the substituted ring A is

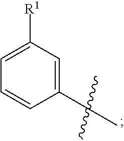

and R¹ is selected from:
C₁-C₆ alkyl optionally substituted with one or more hydroxy; C₃-C₇ cycloalkyl optionally substituted with one or more hydroxy; 3- to 7-membered heterocycloalkyl optionally substituted with one or more hydroxy; C₁-C₆ alkyl substituted with one or more oxo; C₃-C₇ cycloalkyl substituted with one or more oxo; C₁-C₆ alkyl substituted with one or more C₁-C₆ alkoxy; C₃-C₇ cycloalkyl substituted with one or more C₁-C₆ alkoxy; C₁-C₆ alkyl substituted with one or more NR⁸R⁹; 3- to 7-membered heterocycloalkyl substituted with one or more NR⁸R⁹; C₁-C₆ haloalkyl; C₁-C₆ alkoxy; C₁-C₆ haloalkoxy; halo; CN; NO₂; COC₁-C₆ alkyl; CO-C₆-C₁₀ aryl; CO(5- to 10-membered heteroaryl); CO₂C₁-C₆ alkyl; CO₂C₃-C₈ cycloalkyl; OCOC₁-C₆ alkyl; OCOC₆-C₁₀ aryl; OCO(5- to 10-membered heteroaryl); OCO(3- to 7-membered heterocycloalkyl); C₆-C₁₀ aryl; 5- to 10-membered heteroaryl; NH₂; NHC₁-C₆ alkyl; N(C₁-C₆ alkyl)₂; CONR⁸R⁹; SF₅; and S(O₂)C₁-C₆ alkyl.

In some embodiments of the compound of formula AA,

the substituted ring A is

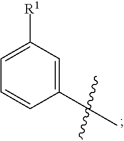

and R¹ is selected from:
1-hydroxy-2-methylpropan-2-yl; methyl; isopropyl; 2-hydroxy-2-propyl; hydroxymethyl; 1-hydroxyethyl; 2-hydroxyethyl; 1-hydroxy-2-propyl; 1-hydroxy-1-cyclopropyl; 1-hydroxy-1-cyclobutyl; 1-hydroxy-1-cyclopentyl; 1-hydroxy-1-cyclohexyl; morpholinyl; 1,3-dioxolan-2-yl; COCH₃; COCH₂CH₃; 2-methoxy-2-propyl; (dimethylamino)methyl; 1-(dimethylamino)ethyl; fluoro; chloro; phenyl; pyridyl; pyrazolyl; and S(O₂)CH₃.

In some embodiments of the compound of formula AA,

the substituted ring A is

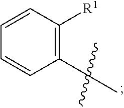

and R¹ is selected from:
C₁-C₆ alkyl optionally substituted with one or more hydroxy; C₃-C₇ cycloalkyl optionally substituted with one or more hydroxy; 3- to 7-membered heterocycloalkyl optionally substituted with one or more hydroxy; C₁-C₆ alkyl substituted with one or more oxo; C₃-C₇ cycloalkyl substituted with one or more oxo; C₁-C₆ alkyl substituted with one or more C₁-C₆ alkoxy; C₃-C₇ cycloalkyl substituted with one or more C₁-C₆ alkoxy; C₁-C₆ alkyl substituted with one or more NR⁸R⁹; 3- to 7-membered heterocycloalkyl substituted with one or more NR⁸R⁹; C₁-C₆ haloalkyl; C₁-C₆ alkoxy; C₁-C₆ haloalkoxy; halo; CN; NO₂; COC₁-C₆ alkyl; CO-C₆-C₁₀ aryl; CO(5- to 10-membered heteroaryl); CO₂C₁-C₆ alkyl; CO₂C₃-C₈ cycloalkyl; OCOC₁-C₆ alkyl; OCOC₆-C₁₀ aryl; OCO(5- to 10-membered heteroaryl); OCO(3- to 7-membered heterocycloalkyl); C₆-C₁₀ aryl; 5- to 10-membered heteroaryl; NH₂; NHC₁-C₆ alkyl; N(C₁-C₆ alkyl)₂; CONR⁸R⁹; SF₅; and S(O₂)C₁-C₆ alkyl.

In some embodiments of the compound of formula AA,

the substituted ring A is

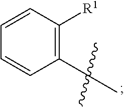

and R¹ is selected from:
1-hydroxy-2-methylpropan-2-yl; methyl; isopropyl; 2-hydroxy-2-propyl; hydroxymethyl; 1-hydroxyethyl; 2-hydroxyethyl; 1-hydroxy-2-propyl; 1-hydroxy-1-cyclopropyl; 1-hydroxy-1-cyclobutyl; 1-hydroxy-1-cyclopentyl; 1-hydroxy-1-cyclohexyl; morpholinyl; 1,3-dioxolan-2-yl; COCH₃; COCH₂CH₃; 2-methoxy-2-propyl; (dimethylamino)methyl; 1-(dimethylamino)ethyl; fluoro; chloro; phenyl; pyridyl; pyrazolyl; and S(O₂)CH₃.

In some embodiments of the compound of formula AA,

the substituted ring A is

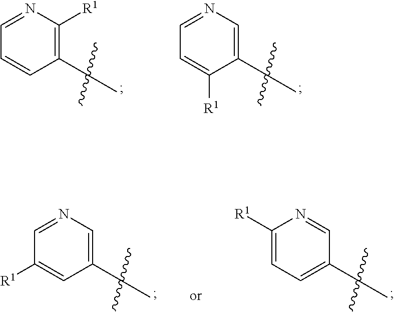

and R¹ is selected from:
C₁-C₆ alkyl optionally substituted with one or more hydroxy; C₃-C₇ cycloalkyl optionally substituted with one or more hydroxy; 3- to 7-membered heterocycloalkyl optionally substituted with one or more hydroxy; C₁-C₆ alkyl substituted with one or more oxo; C₃-C₇ cycloalkyl substituted with one or more oxo; C₁-C₆ alkyl substituted with one or more C₁-C₆ alkoxy; C₃-C₇ cycloalkyl substituted with one or more C₁-C₆ alkoxy; C₁-C₆ alkyl substituted with one or more NR⁸R⁹; 3- to 7-membered heterocycloalkyl substituted with one or more NR⁸R⁹; C₁-C₆ haloalkyl; C₁-C₆ alkoxy; C₁-C₆ haloalkoxy; halo; CN; NO₂; COC₁-C₆ alkyl; CO-C₆-C₁₀ aryl; CO(5- to 10-membered heteroaryl); CO₂C₁-C₆ alkyl; CO₂C₃-C₈ cycloalkyl; OCOC₁-C₆ alkyl; OCOC₆-C₁₀ aryl; OCO(5- to 10-membered heteroaryl); OCO(3- to 7-membered heterocycloalkyl); C₆-C₁₀ aryl; 5- to 10-membered heteroaryl; NH₂; NHC₁-C₆ alkyl; N(C₁-C₆ alkyl)₂; CONR⁸R⁹; SF₅; and S(O₂)C₁-C₆ alkyl.

In some embodiments of the compound of formula AA,

the substituted ring A is

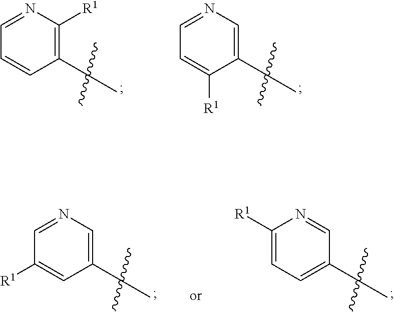

and R¹ is selected from:
1-hydroxy-2-methylpropan-2-yl; methyl; isopropyl; 2-hydroxy-2-propyl; hydroxymethyl; 1-hydroxyethyl; 2-hydroxyethyl; 1-hydroxy-2-propyl; 1-hydroxy-1-cyclopropyl; 1-hydroxy-1-cyclobutyl; 1-hydroxy-1-cyclopentyl; 1-hydroxy-1-cyclohexyl; morpholinyl; 1,3-dioxolan-2-yl; COCH₃; COCH₂CH₃; 2-methoxy-2-propyl; (dimethylamino)methyl; 1-(dimethylamino)ethyl; fluoro; chloro; phenyl; pyridyl; pyrazolyl; and S(O₂)CH₃.

the substituted ring A is

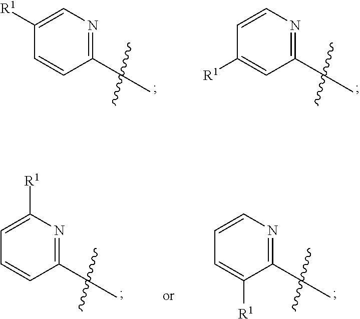

and R¹ is selected from:
C₁-C₆ alkyl optionally substituted with one or more hydroxy; C₃-C₇ cycloalkyl optionally substituted with one or more hydroxy; 3- to 7-membered heterocycloalkyl optionally substituted with one or more hydroxy; C₁-C₆ alkyl substituted with one or more oxo; C₃-C₇ cycloalkyl substituted with one or more oxo; C₁-C₆ alkyl substituted with one or more C₁-C₆ alkoxy; C₃-C₇ cycloalkyl substituted with one or more C₁-C₆ alkoxy; C₁-C₆ alkyl substituted with one or more NR⁸R⁹; 3- to 7-membered heterocycloalkyl substituted with one or more NR⁸R⁹; C₁-C₆ haloalkyl; C₁-C₆ alkoxy; C₁-C₆ haloalkoxy; halo; CN; NO₂; COC₁-C₆ alkyl; CO-C₆-C₁₀ aryl; CO(5- to 10-membered heteroaryl); CO₂C₁-C₆ alkyl; CO₂C₃-C₈ cycloalkyl; OCOC₁-C₆ alkyl; OCOC₆-C₁₀ aryl; OCO(5- to 10-membered heteroaryl); OCO(3- to 7-membered heterocycloalkyl); C₆-C₁₀ aryl; 5- to 10-membered heteroaryl; NH₂; NHC₁-C₆ alkyl; N(C₁-C₆ alkyl)₂; CONR⁸R⁹; SF₅; and S(O₂)C₁-C₆ alkyl.

In some embodiments of the compound of formula AA,

the substituted ring A is

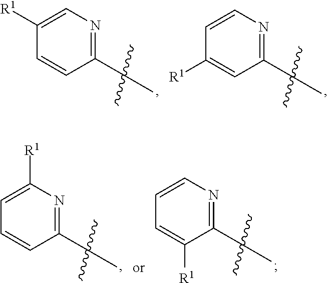

and R¹ is selected from:
1-hydroxy-2-methylpropan-2-yl; methyl; isopropyl; 2-hydroxy-2-propyl; hydroxymethyl; 1-hydroxyethyl; 2-hydroxyethyl; 1-hydroxy-2-propyl; 1-hydroxy-1-cyclopropyl; 1-hydroxy-1-cyclobutyl; 1-hydroxy-1-cyclopentyl; 1-hydroxy-1-cyclohexyl; morpholinyl; 1,3-dioxolan-2-yl; COCH₃; COCH₂CH₃; 2-methoxy-2-propyl; (dimethylamino)methyl; 1-(dimethylamino)ethyl; fluoro; chloro; phenyl; pyridyl; pyrazolyl; and S(O₂)CH₃.

In some embodiments of the compound of formula AA,

the substituted ring A is

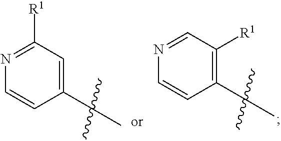

and R¹ is selected from:
C₁-C₆ alkyl optionally substituted with one or more hydroxy; C₃-C₇ cycloalkyl optionally substituted with one or more hydroxy; 3- to 7-membered heterocycloalkyl optionally substituted with one or more hydroxy; C₁-C₆ alkyl substituted with one or more oxo; C₃-C₇ cycloalkyl substituted with one or more oxo; C₁-C₆ alkyl substituted with one or more C₁-C₆ alkoxy; C₃-C₇ cycloalkyl substituted with one or more C₁-C₆ alkoxy; C₁-C₆ alkyl substituted with one or more NR⁸R⁹; 3- to 7-membered heterocycloalkyl substituted with one or more NR⁸R⁹; C₁-C₆ haloalkyl; C₁-C₆ alkoxy; C₁-C₆ haloalkoxy; halo; CN; NO₂; COC₁-C₆ alkyl; CO-C₆-C₁₀ aryl; CO-5- to 10-membered heteroaryl; CO₂C₁-C₆ alkyl; CO₂C₃-C₈ cycloalkyl; OCOC₁-C₆ alkyl; OCOC₆-C₁₀ aryl; OCO(5- to 10-membered heteroaryl); OCO(3- to 7-membered heterocycloalkyl); C₆-C₁₀ aryl; 5- to 10-membered heteroaryl; NH₂; NHC₁-C₆ alkyl; N(C₁-C₆ alkyl)₂; CONR⁸R⁹; SF₅; and S(O₂)C₁-C₆ alkyl.

In some embodiments of the compound of formula AA,

the substituted ring A is

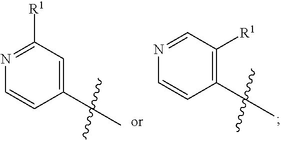

and R¹ is selected from:
1-hydroxy-2-methylpropan-2-yl; methyl; isopropyl; 2-hydroxy-2-propyl; hydroxymethyl; 1-hydroxyethyl; 2-hydroxyethyl; 1-hydroxy-2-propyl; 1-hydroxy-1-cyclopropyl; 1-hydroxy-1-cyclobutyl; 1-hydroxy-1-cyclopentyl; 1-hydroxy-1-cyclohexyl; morpholinyl; 1,3-dioxolan-2-yl; COCH₃; COCH₂CH₃; 2-methoxy-2-propyl; (dimethylamino)methyl; 1-(dimethylamino)ethyl; fluoro; chloro; phenyl; pyridyl; pyrazolyl; and S(O₂)CH₃.

In some embodiments of the compound of formula AA,

the substituted ring A is

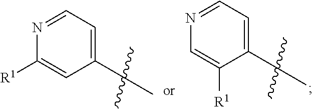

and R¹ is selected from:
C₁-C₆ alkyl optionally substituted with one or more hydroxy; C₃-C₇ cycloalkyl optionally substituted with one or more hydroxy; 3- to 7-membered heterocycloalkyl optionally substituted with one or more hydroxy; C₁-C₆ alkyl substituted with one or more oxo; C₃-C₇ cycloalkyl substituted with one or more oxo; C₁-C₆ alkyl substituted with one or more C₁-C₆ alkoxy; C₃-C₇ cycloalkyl substituted with one or more C₁-C₆ alkoxy; C₁-C₆ alkyl substituted with one or more NR⁸R⁹; 3- to 7-membered heterocycloalkyl substituted with one or more NR⁸R⁹; C₁-C₆ haloalkyl; C₁-C₆ alkoxy; C₁-C₆ haloalkoxy; halo; CN; NO₂; COC₁-C₆ alkyl; CO-C₆-C₁₀ aryl; CO(5- to 10-membered heteroaryl); CO₂C₁-C₆ alkyl; CO₂C₃-C₈ cycloalkyl; OCOC₁-C₆ alkyl; OCOC₆-C₁₀ aryl; OCO(5- to 10-membered heteroaryl); OCO(3- to 7-membered heterocycloalkyl); C₆-C₁₀ aryl; 5- to 10-membered heteroaryl; NH₂; NHC₁-C₆ alkyl; N(C₁-C₆ alkyl)₂; CONR⁸R⁹; SF₅; and S(O₂)C₁-C₆ alkyl.

In some embodiments of the compound of formula AA,

the substituted ring A is

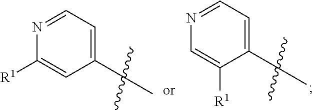

and R¹ is selected from:
1-hydroxy-2-methylpropan-2-yl; methyl; isopropyl; 2-hydroxy-2-propyl; hydroxymethyl; 1-hydroxyethyl; 2-hydroxyethyl; 1-hydroxy-2-propyl; 1-hydroxy-1-cyclopropyl; 1-hydroxy-1-cyclobutyl; 1-hydroxy-1-cyclopentyl; 1-hydroxy-1-cyclohexyl; morpholinyl; 1,3-dioxolan-2-yl; COCH₃; COCH₂CH₃; 2-methoxy-2-propyl; (dimethylamino)methyl; 1-(dimethylamino)ethyl; fluoro; chloro; phenyl; pyridyl; pyrazolyl; and S(O₂)CH₃.

In some embodiments of the compound of formula AA,

the substituted ring A is

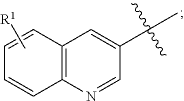

and R¹ is selected from:
C₁-C₆ alkyl optionally substituted with one or more hydroxy; C₃-C₇ cycloalkyl optionally substituted with one or more hydroxy; 3- to 7-membered heterocycloalkyl optionally substituted with one or more hydroxy; C₁-C₆ alkyl substituted with one or more oxo; C₃-C₇ cycloalkyl substituted with one or more oxo; C₁-C₆ alkyl substituted with one or more C₁-C₆ alkoxy; C₃-C₇ cycloalkyl substituted with one or more C₁-C₆ alkoxy; C₁-C₆ haloalkyl; C₁-C₆ alkoxy; C₁-C₆ haloalkoxy; halo; CN; NO₂; COC₁-C₆ alkyl; CO-C₆-C₁₀ aryl; CO(5- to 10-membered heteroaryl); CO₂C₁-C₆ alkyl; CO₂C₃-C₈ cycloalkyl; OCOC₁-C₆ alkyl; OCOC₆-C₁₀ aryl; OCO(5- to 10-membered heteroaryl); OCO(3- to 7-membered heterocycloalkyl); C₆-C₁₀ aryl; 5- to 10-membered heteroaryl; NH₂; NHC₁-C₆ alkyl; N(C₁-C₆ alkyl)₂; CONR⁸R⁹; SF₅; C₁-C₆ alkyl substituted with one or more NR⁸R⁹; and S(O₂)C₁-C₆ alkyl.

the substituted ring A is

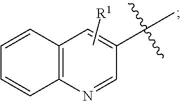

and R¹ is selected from:
1-hydroxy-2-methylpropan-2-yl; methyl; isopropyl; 2-hydroxy-2-propyl; hydroxymethyl; 1-hydroxyethyl; 2-hydroxyethyl; 1-hydroxy-2-propyl; 1-hydroxy-1-cyclopropyl; 1-hydroxy-1-cyclobutyl; 1-hydroxy-1-cyclopentyl; 1-hydroxy-1-cyclohexyl; morpholinyl; (dimethylamino)methyl; 1,3-dioxolan-2-yl; COCH₃; COCH₂CH₃; 2-methoxy-2-propyl; fluoro; chloro; phenyl; pyridyl; pyrazolyl; and S(O₂)CH₃.

the substituted ring A is

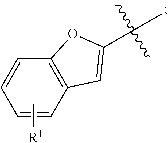

and R¹ is selected from:
C₁-C₆ alkyl optionally substituted with one or more hydroxy; C₃-C₇ cycloalkyl optionally substituted with one or more hydroxy; 3- to 7-membered heterocycloalkyl optionally substituted with one or more hydroxy; C₁-C₆ alkyl substituted with one or more oxo; C₃-C₇ cycloalkyl substituted with one or more oxo; C₁-C₆ alkyl substituted with one or more C₁-C₆ alkoxy; C₃-C₇ cycloalkyl substituted with one or more C₁-C₆ alkoxy; C₁-C₆ haloalkyl; C₁-C₆ alkoxy; C₁-C₆ haloalkoxy; halo; CN; NO₂; COC₁-C₆ alkyl; CO-C₆-C₁₀ aryl; CO(5- to 10-membered heteroaryl); CO₂C₁-C₆ alkyl; CO₂C₃-C₈ cycloalkyl; OCOC₁-C₆ alkyl; OCOC₆-C₁₀ aryl; OCO(5- to 10-membered heteroaryl); OCO(3- to 7-membered heterocycloalkyl); C₆-C₁₀ aryl; 5- to 10-membered heteroaryl; NH₂; NHC₁-C₆ alkyl; N(C₁-C₆ alkyl)₂; CONR⁸R⁹; SF₅; C₁-C₆ alkyl substituted with one or more NR⁸R⁹; and S(O₂)C₁-C₆ alkyl.

the substituted ring A is

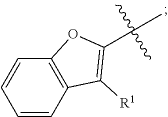

and R¹ is selected from:
1-hydroxy-2-methylpropan-2-yl; methyl; isopropyl; 2-hydroxy-2-propyl; hydroxymethyl; 1-hydroxyethyl; 2-hydroxyethyl; 1-hydroxy-2-propyl; 1-hydroxy-1-cyclopropyl; 1-hydroxy-1-cyclobutyl; 1-hydroxy-1-cyclopentyl; 1-hydroxy-1-cyclohexyl; morpholinyl; 1,3-dioxolan-2-yl; COCH₃; COCH₂CH₃; 2-methoxy-2-propyl; fluoro; chloro; phenyl; pyridyl; pyrazolyl; (dimethylamino)methyl; and S(O₂)CH₃.

In some embodiments of the compound of formula AA,

the substituted ring A is

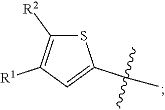

and R¹ and R² are one of the following combinations:
R¹ is C₁-C₆ alkyl optionally substituted with one or more hydroxy, and R² is C₁-C₆ alkyl optionally substituted with one or more hydroxy;
R¹ is C₁-C₆ alkyl optionally substituted with one or more hydroxy, and R² is C₆-C₁₀ aryl;
R¹ is C₁-C₆ alkyl optionally substituted with one or more hydroxy, and R² is 5- to 10-membered heteroaryl;
R¹ is C₁-C₆ alkyl optionally substituted with one or more hydroxy, and R² is SF₅;
R¹ is C₁-C₆ alkyl optionally substituted with one or more hydroxy, and R² is S(O₂)C₁-C₆ alkyl;
R¹ is C₁-C₆ alkyl optionally substituted with one or more hydroxy, and R² is halo;
R¹ is C₃-C₇ cycloalkyl optionally substituted with one or more hydroxy, and R² is C₁-C₆ alkyl;
R¹ is 3- to 7-membered heterocycloalkyl optionally substituted with one or more hydroxy, and R² is C₁-C₆ alkyl;
R¹ is 3- to 7-membered heterocycloalkyl optionally substituted with one or more hydroxy, and R² is halo;
R¹ is C₁-C₆ alkyl optionally substituted with one or more oxo, and R² is methyl;
R¹ is C₁-C₆ alkyl optionally substituted with one or more C₁-C₆ alkoxy, and R² is C₁-C₆ alkyl;
R¹ is C₁-C₆ alkyl optionally substituted with one or more NR⁸R⁹, and R² is C₁-C₆ alkyl;
R¹ is C₁-C₆ alkyl optionally substituted with one or more NR⁸R⁹, and R² is halo;
R² is C₁-C₆ alkyl optionally substituted with one or more hydroxy, and R¹ is C₆-C₁₀ aryl;
R² is C₁-C₆ alkyl optionally substituted with one or more hydroxy, and R¹ is 5- to 10-membered heteroaryl;
R² is C₁-C₆ alkyl optionally substituted with one or more hydroxy, and R¹ is SF₅.
R² is C₁-C₆ alkyl optionally substituted with one or more hydroxy, and R¹ is S(O₂)C₁-C₆ alkyl;
R² is C₁-C₆ alkyl optionally substituted with one or more hydroxy, and R¹ is halo;
R² is C₃-C₇ cycloalkyl optionally substituted with one or more hydroxy, and R¹ is C₁-C₆ alkyl;
R² is 3- to 7-membered heterocycloalkyl optionally substituted with one or more hydroxy, and R¹ is C₁-C₆ alkyl;
R² is 3- to 7-membered heterocycloalkyl optionally substituted with one or more hydroxy, and R¹ is halo;
R² is C₁-C₆ alkyl optionally substituted with one or more oxo, and R¹ is methyl;
R² is C₁-C₆ alkyl optionally substituted with one or more C₁-C₆ alkoxy, and R¹ is C₁-C₆ alkyl;
R² is C₁-C₆ alkyl optionally substituted with one or more NR⁸R⁹, and R¹ is C₁-C₆ alkyl;
R² is C₁-C₆ alkyl optionally substituted with one or more NR⁸R⁹, and R¹ is halo.

In some embodiments of the compound of formula AA,

the substituted ring A is

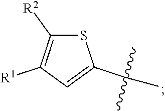

and R¹ and R² are one of the following combinations:
R¹ is 1-hydroxy-2-methylpropan-2-yl, and R² is methyl;
R¹ is 2-hydroxy-2-propyl and R² is methyl;
R¹ is 2-hydroxy-2-propyl and R² is isopropyl;
R¹ is 2-hydroxy-2-propyl and R² is 2-hydroxy-2-propyl;
R¹ is 2-hydroxy-2-propyl and R² is 1-hydroxyethyl;
R¹ is hydroxymethyl and R² is methyl;
R¹ is 1-hydroxyethyl and R² is methyl;
R¹ is 2-hydroxyethyl and R² is methyl;
R¹ is 1-hydroxy-2-propyl and R² is methyl;
R¹ is 2-hydroxy-2-propyl and R² is phenyl;
R¹ is 2-hydroxy-2-propyl and R² is pyridyl;
R¹ is 2-hydroxy-2-propyl and R² is pyrazolyl;
R¹ is 2-hydroxy-2-propyl, and R² is S(O₂)CH₃;
R¹ is 2-hydroxy-2-propyl and R² is chloro;
R¹ is 2-hydroxy-2-propyl and R² is fluoro;
R¹ is 1-hydroxy-1-cyclopropyl, and R² is methyl;
R¹ is 1-hydroxy-1-cyclobutyl, and R² is methyl;
R¹ is 1-hydroxy-1-cyclopentyl, and R² is methyl;
R¹ is 1-hydroxy-1-cyclohexyl, and R² is methyl;
R¹ is morpholinyl, and R² is methyl;
R¹ is 1,3-dioxolan-2-yl, and R² is methyl;
R¹ is 1,3-dioxolan-2-yl, and R² is fluoro;
R¹ is 1,3-dioxolan-2-yl, and R² is chloro;
R¹ is COCH₃, and R² is methyl;
R¹ is 2-methoxy-2-propyl, and R² is methyl;
R² is (dimethylamino)methyl, and R¹ is methyl.
R² is 1-hydroxy-2-methylpropan-2-yl, and R¹ is methyl;
R² is 2-hydroxy-2-propyl and R¹ is methyl;
R² is 2-hydroxy-2-propyl and R¹ is isopropyl;
R² is 2-hydroxy-2-propyl and R¹ is 1-hydroxyethyl;
R² is hydroxymethyl and R¹ is methyl;
R² is 1-hydroxyethyl and R¹ is methyl;
R² is 2-hydroxyethyl and R¹ is methyl;
R² is 1-hydroxy-2-propyl and R¹ is methyl;
R² is 2-hydroxy-2-propyl and R¹ is phenyl;
R² is C₁-C₆ alkyl optionally substituted with one or more hydroxy, and R¹ is 5- to 10-membered heteroaryl;
R² is 2-hydroxy-2-propyl and R¹ is pyridyl;
R² is 2-hydroxy-2-propyl and R¹ is pyrazolyl;
R² is C₁-C₆ alkyl optionally substituted with one or more hydroxy, and R¹ is S(O₂)CH₃;
R² is 2-hydroxy-2-propyl and R¹ is chloro;
R² is 2-hydroxy-2-propyl and R¹ is fluoro;
R² is C₃-C₇ cycloalkyl optionally substituted with one or more hydroxy, and R¹ is C₁-C₆ alkyl;
R² is 1-hydroxy-1-cyclopropyl, and R¹ is methyl;
R² is 1-hydroxy-1-cyclobutyl, and R¹ is methyl;
R² is 1-hydroxy-1-cyclopentyl, and R¹ is methyl;
R² is 1-hydroxy-1-cyclohexyl, and R¹ is methyl;
R² is morpholinyl, and R¹ is methyl;
R² is 1,3-dioxolan-2-yl, and R¹ is methyl;
R² is 1,3-dioxolan-2-yl, and R¹ is fluoro;
R² is 1,3-dioxolan-2-yl, and R¹ is chloro;
R² is C₁-C₆ alkyl optionally substituted with one or more oxo, and R¹ is methyl;
R² is COCH₃, and R¹ is methyl; or
R² is 2-methoxy-2-propyl, and R¹ is methyl.

In some embodiments, of the compound of formula AA,

the substituted ring A is

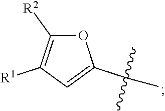

and R¹ and R² are one of the following combinations:
R¹ is C₁-C₆ alkyl optionally substituted with one or more hydroxy, and R² is C₁-C₆ alkyl optionally substituted with one or more hydroxy;
R¹ is C₁-C₆ alkyl optionally substituted with one or more hydroxy, and R² is C₆-C₁₀ aryl;
R¹ is C₁-C₆ alkyl optionally substituted with one or more hydroxy, and R² is 5- to 10-membered heteroaryl;
R¹ is C₁-C₆ alkyl optionally substituted with one or more hydroxy, and R² is SF₅;
R¹ is C₁-C₆ alkyl optionally substituted with one or more hydroxy, and R² is S(O₂)C₁-C₆ alkyl;
R¹ is C₁-C₆ alkyl optionally substituted with one or more hydroxy, and R² is halo;
R¹ is C₃-C₇ cycloalkyl optionally substituted with one or more hydroxy, and R² is C₁-C₆ alkyl;
R¹ is 3- to 7-membered heterocycloalkyl optionally substituted with one or more hydroxy, and R² is C₁-C₆ alkyl;
R¹ is 3- to 7-membered heterocycloalkyl optionally substituted with one or more hydroxy, and R² is halo;
R¹ is C₁-C₆ alkyl optionally substituted with one or more oxo, and R² is methyl;
R¹ is C₁-C₆ alkyl optionally substituted with one or more C₁-C₆ alkoxy, and R² is C₁-C₆ alkyl;
R¹ is C₁-C₆ alkyl optionally substituted with one or more NR⁸R⁹, and R² is C₁-C₆ alkyl;
R¹ is C₁-C₆ alkyl optionally substituted with one or more NR⁸R⁹, and R² is halo;
R² is C₁-C₆ alkyl optionally substituted with one or more hydroxy, and R¹ is C₆-C₁₀ aryl;
R² is C₁-C₆ alkyl optionally substituted with one or more hydroxy, and R¹ is 5- to 10-membered heteroaryl;
R² is C₁-C₆ alkyl optionally substituted with one or more hydroxy, and R¹ is SF₅.
R² is C₁-C₆ alkyl optionally substituted with one or more hydroxy, and R¹ is S(O₂)C₁-C₆ alkyl;
R² is C₁-C₆ alkyl optionally substituted with one or more hydroxy, and R¹ is halo;
R² is C₃-C₇ cycloalkyl optionally substituted with one or more hydroxy, and R¹ is C₁-C₆ alkyl;
R² is 3- to 7-membered heterocycloalkyl optionally substituted with one or more hydroxy, and R¹ is C₁-C₆ alkyl;
R² is 3- to 7-membered heterocycloalkyl optionally substituted with one or more hydroxy, and R¹ is halo;
R² is C₁-C₆ alkyl optionally substituted with one or more oxo, and R¹ is methyl; or
R² is C₁-C₆ alkyl optionally substituted with one or more C₁-C₆ alkoxy, and R¹ is C₁-C₆ alkyl.
R² is C₁-C₆ alkyl optionally substituted with one or more NR⁸R⁹, and R¹ is C₁-C₆ alkyl; or
R² is C₁-C₆ alkyl optionally substituted with one or more NR⁸R⁹, and R¹ is halo.

In some embodiments, of the compound of formula AA,

the substituted ring A is

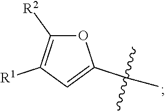

and R¹ and R² are one of the following combinations:
R¹ is 1-hydroxy-2-methylpropan-2-yl, and R² is methyl;
R¹ is 2-hydroxy-2-propyl and R² is methyl;
R¹ is 2-hydroxy-2-propyl and R² is isopropyl;
R¹ is 2-hydroxy-2-propyl and R² is 2-hydroxy-2-propyl;
R¹ is 2-hydroxy-2-propyl and R² is 1-hydroxyethyl;
R¹ is hydroxymethyl and R² is methyl;
R¹ is 1-hydroxyethyl and R² is methyl;
R¹ is 2-hydroxyethyl and R² is methyl;
R¹ is 1-hydroxy-2-propyl and R² is methyl;
R¹ is 2-hydroxy-2-propyl and R² is phenyl;
R¹ is 2-hydroxy-2-propyl and R² is pyridyl;
R¹ is 2-hydroxy-2-propyl and R² is pyrazolyl;
R¹ is 2-hydroxy-2-propyl, and R² is S(O₂)CH₃;
R¹ is 2-hydroxy-2-propyl and R² is chloro;
R¹ is 2-hydroxy-2-propyl and R² is fluoro;
R¹ is 1-hydroxy-1-cyclopropyl, and R² is methyl;
R¹ is 1-hydroxy-1-cyclobutyl, and R² is methyl;
R¹ is 1-hydroxy-1-cyclopentyl, and R² is methyl;
R¹ is 1-hydroxy-1-cyclohexyl, and R² is methyl;
R¹ is morpholinyl, and R² is methyl;
R¹ is 1,3-dioxolan-2-yl, and R² is methyl;
R¹ is 1,3-dioxolan-2-yl, and R² is fluoro;
R¹ is 1,3-dioxolan-2-yl, and R² is chloro;
R¹ is COCH₃, and R² is methyl;
R¹ is 2-methoxy-2-propyl, and R² is methyl;
R¹ is (dimethylamino)methyl, and R² is methyl.
R² is 1-hydroxy-2-methylpropan-2-yl, and R¹ is methyl;
R² is 2-hydroxy-2-propyl and R¹ is methyl;
R² is 2-hydroxy-2-propyl and R¹ is isopropyl;
R² is 2-hydroxy-2-propyl and R¹ is 1-hydroxyethyl;
R² is hydroxymethyl and R¹ is methyl;
R² is 1-hydroxyethyl and R¹ is methyl;
R² is 2-hydroxyethyl and R¹ is methyl;
R² is 1-hydroxy-2-propyl and R¹ is methyl;
R² is 2-hydroxy-2-propyl and R¹ is phenyl;
R² is C₁-C₆ alkyl optionally substituted with one or more hydroxy, and R¹ is 5- to 10-membered heteroaryl;
R² is 2-hydroxy-2-propyl and R¹ is pyridyl;
R² is 2-hydroxy-2-propyl and R¹ is pyrazolyl;
R² is C₁-C₆ alkyl optionally substituted with one or more hydroxy, and R¹ is S(O₂)CH₃;
R² is 2-hydroxy-2-propyl and R¹ is chloro;
R² is 2-hydroxy-2-propyl and R¹ is fluoro;
R² is C₃-C₇ cycloalkyl optionally substituted with one or more hydroxy, and R¹ is C₁-C₆ alkyl;
R² is 1-hydroxy-1-cyclopropyl, and R¹ is methyl;
R² is 1-hydroxy-1-cyclobutyl, and R¹ is methyl;
R² is 1-hydroxy-1-cyclopentyl, and R¹ is methyl;
R² is 1-hydroxy-1-cyclohexyl, and R¹ is methyl;
R² is morpholinyl, and R¹ is methyl;
R² is 1,3-dioxolan-2-yl, and R¹ is methyl;
R² is 1,3-dioxolan-2-yl, and R¹ is fluoro;
R² is 1,3-dioxolan-2-yl, and R¹ is chloro;
R² is C₁-C₆ alkyl optionally substituted with one or more oxo, and R¹ is methyl;
R² is (dimethylamino)methyl, and R¹ is methyl;
R² is COCH₃, and R¹ is methyl; or
R² is 2-methoxy-2-propyl, and R¹ is methyl.

In some embodiments, of the compound of formula AA,

the substituted ring A is

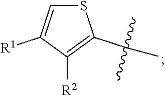

and R¹ and R² are one of the following combinations:
R¹ is C₁-C₆ alkyl optionally substituted with one or more hydroxy, and R² is C₁-C₆ alkyl optionally substituted with one or more hydroxy;
R¹ is C₁-C₆ alkyl optionally substituted with one or more hydroxy, and R² is C₆-C₁₀ aryl;
R¹ is C₁-C₆ alkyl optionally substituted with one or more hydroxy, and R² is 5- to 10-membered heteroaryl;
R¹ is C₁-C₆ alkyl optionally substituted with one or more hydroxy, and R² is SF₅;
R¹ is C₁-C₆ alkyl optionally substituted with one or more hydroxy, and R² is S(O₂)C₁-C₆ alkyl;
R¹ is C₁-C₆ alkyl optionally substituted with one or more hydroxy, and R² is halo;
R¹ is C₃-C₇ cycloalkyl optionally substituted with one or more hydroxy, and R² is C₁-C₆ alkyl;
R¹ is 3- to 7-membered heterocycloalkyl optionally substituted with one or more hydroxy, and R² is C₁-C₆ alkyl;
R¹ is 3- to 7-membered heterocycloalkyl optionally substituted with one or more hydroxy, and R² is halo;
R¹ is C₁-C₆ alkyl optionally substituted with one or more oxo, and R² is methyl;
R¹ is C₁-C₆ alkyl optionally substituted with one or more C₁-C₆ alkoxy, and R² is C₁-C₆ alkyl;
R¹ is C₁-C₆ alkyl optionally substituted with one or more NR⁸R⁹, and R² is C₁-C₆ alkyl;
R¹ is C₁-C₆ alkyl optionally substituted with one or more NR⁸R⁹, and R² is halo;
R² is C₁-C₆ alkyl optionally substituted with one or more hydroxy, and R¹ is C₆-C₁₀ aryl;
R² is C₁-C₆ alkyl optionally substituted with one or more hydroxy, and R¹ is 5- to 10-membered heteroaryl;
R² is C₁-C₆ alkyl optionally substituted with one or more hydroxy, and R¹ is SF₅.
R² is C₁-C₆ alkyl optionally substituted with one or more hydroxy, and R¹ is S(O₂)C₁-C₆ alkyl;
R² is C₁-C₆ alkyl optionally substituted with one or more hydroxy, and R¹ is halo;
R² is C₃-C₇ cycloalkyl optionally substituted with one or more hydroxy, and R¹ is C₁-C₆ alkyl;
R² is 3- to 7-membered heterocycloalkyl optionally substituted with one or more hydroxy, and R¹ is C₁-C₆ alkyl;
R² is 3- to 7-membered heterocycloalkyl optionally substituted with one or more hydroxy, and R¹ is halo;
R² is C₁-C₆ alkyl optionally substituted with one or more NR⁸R⁹, and R¹ is C₁-C₆ alkyl;
R² is C₁-C₆ alkyl optionally substituted with one or more NR⁸R⁹, and R¹ is halo;
R² is C₁-C₆ alkyl optionally substituted with one or more oxo, and R¹ is methyl; or
R² is C₁-C₆ alkyl optionally substituted with one or more C₁-C₆ alkoxy, and R¹ is C₁-C₆ alkyl.

In some embodiments, of the compound of formula AA,

the substituted ring A is

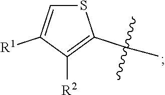

and R¹ and R² are one of the following combinations:
R¹ is 1-hydroxy-2-methylpropan-2-yl, and R² is methyl;
R¹ is 2-hydroxy-2-propyl and R² is methyl;
R¹ is 2-hydroxy-2-propyl and R² is isopropyl;
R¹ is 2-hydroxy-2-propyl and R² is 2-hydroxy-2-propyl;
R¹ is 2-hydroxy-2-propyl and R² is 1-hydroxyethyl;
R¹ is hydroxymethyl and R² is methyl;
R¹ is 1-hydroxyethyl and R² is methyl;
R¹ is 2-hydroxyethyl and R² is methyl;
R¹ is 1-hydroxy-2-propyl and R² is methyl;
R¹ is 2-hydroxy-2-propyl and R² is phenyl;
R¹ is 2-hydroxy-2-propyl and R² is pyridyl;
R¹ is 2-hydroxy-2-propyl and R² is pyrazolyl;
R¹ is 2-hydroxy-2-propyl, and R² is S(O₂)CH₃;
R¹ is 2-hydroxy-2-propyl and R² is chloro;
R¹ is 2-hydroxy-2-propyl and R² is fluoro;
R¹ is 1-hydroxy-1-cyclopropyl, and R² is methyl;
R¹ is 1-hydroxy-1-cyclobutyl, and R² is methyl;
R¹ is 1-hydroxy-1-cyclopentyl, and R² is methyl;
R¹ is 1-hydroxy-1-cyclohexyl, and R² is methyl;
R¹ is morpholinyl, and R² is methyl;
R¹ is 1,3-dioxolan-2-yl, and R² is methyl;
R¹ is 1,3-dioxolan-2-yl, and R² is fluoro;
R¹ is 1,3-dioxolan-2-yl, and R² is chloro;
R¹ is COCH₃, and R² is methyl;
R¹ is 2-methoxy-2-propyl, and R² is methyl;
R¹ is (dimethylamino)methyl, and R² is methyl;
R² is 1-hydroxy-2-methylpropan-2-yl, and R¹ is methyl;
R² is 2-hydroxy-2-propyl and R¹ is methyl;
R² is 2-hydroxy-2-propyl and R¹ is isopropyl;
R² is 2-hydroxy-2-propyl and R¹ is 1-hydroxyethyl;
R² is hydroxymethyl and R¹ is methyl;
R² is 1-hydroxyethyl and R¹ is methyl;
R² is 2-hydroxyethyl and R¹ is methyl;
R² is 1-hydroxy-2-propyl and R¹ is methyl;
R² is 2-hydroxy-2-propyl and R¹ is phenyl;
R² is C₁-C₆ alkyl optionally substituted with one or more hydroxy, and R¹ is 5- to 10-membered heteroaryl;
R² is 2-hydroxy-2-propyl and R¹ is pyridyl;
R² is 2-hydroxy-2-propyl and R¹ is pyrazolyl;
R² is C₁-C₆ alkyl optionally substituted with one or more hydroxy, and R¹ is S(O₂)CH₃;
R² is 2-hydroxy-2-propyl and R¹ is chloro;
R² is 2-hydroxy-2-propyl and R¹ is fluoro;
R² is C₃-C₇ cycloalkyl optionally substituted with one or more hydroxy, and R¹ is C₁-C₆ alkyl;
R² is 1-hydroxy-1-cyclopropyl, and R¹ is methyl;
R² is 1-hydroxy-1-cyclobutyl, and R¹ is methyl;
R² is 1-hydroxy-1-cyclopentyl, and R¹ is methyl;
R² is 1-hydroxy-1-cyclohexyl, and R¹ is methyl;
R² is morpholinyl, and R¹ is methyl;
R² is 1,3-dioxolan-2-yl, and R¹ is methyl;
R² is 1,3-dioxolan-2-yl, and R¹ is fluoro;
R² is 1,3-dioxolan-2-yl, and R¹ is chloro;
R² is C₁-C₆ alkyl optionally substituted with one or more oxo, and R¹ is methyl;
R² is (dimethylamino)methyl, and R¹ is methyl;
R² is COCH₃, and R¹ is methyl; or
R² is 2-methoxy-2-propyl, and R¹ is methyl.

In some embodiments, of the compound of formula AA,

the substituted ring A is

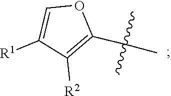

and R¹ and R² are one of the following combinations:
R¹ is C₁-C₆ alkyl optionally substituted with one or more hydroxy, and R² is C₁-C₆ alkyl optionally substituted with one or more hydroxy;
R¹ is C₁-C₆ alkyl optionally substituted with one or more hydroxy, and R² is C₆-C₁₀ aryl;
R¹ is C₁-C₆ alkyl optionally substituted with one or more hydroxy, and R² is 5- to 10-membered heteroaryl;
R¹ is C₁-C₆ alkyl optionally substituted with one or more hydroxy, and R² is SF₅;
R¹ is C₁-C₆ alkyl optionally substituted with one or more hydroxy, and R² is S(O₂)C₁-C₆ alkyl;
R¹ is C₁-C₆ alkyl optionally substituted with one or more hydroxy, and R² is halo;
R¹ is C₃-C₇ cycloalkyl optionally substituted with one or more hydroxy, and R² is C₁-C₆ alkyl;
R¹ is 3- to 7-membered heterocycloalkyl optionally substituted with one or more hydroxy, and R² is C₁-C₆ alkyl;
R¹ is 3- to 7-membered heterocycloalkyl optionally substituted with one or more hydroxy, and R² is halo;
R¹ is C₁-C₆ alkyl optionally substituted with one or more oxo, and R² is methyl;
R¹ is C₁-C₆ alkyl optionally substituted with one or more C₁-C₆ alkoxy, and R² is C₁-C₆ alkyl;
R¹ is C₁-C₆ alkyl optionally substituted with one or more NR⁸R⁹, and R² is C₁-C₆ alkyl;
R¹ is C₁-C₆ alkyl optionally substituted with one or more NR⁸R⁹, and R² is halo;
R² is C₁-C₆ alkyl optionally substituted with one or more hydroxy, and R¹ is C₆-C₁₀ aryl;
R² is C₁-C₆ alkyl optionally substituted with one or more hydroxy, and R¹ is 5- to 10-membered heteroaryl;
R² is C₁-C₆ alkyl optionally substituted with one or more hydroxy, and R¹ is SF₅.
R² is C₁-C₆ alkyl optionally substituted with one or more hydroxy, and R¹ is S(O₂)C₁-C₆ alkyl;
R² is C₁-C₆ alkyl optionally substituted with one or more hydroxy, and R¹ is halo;
R² is C₃-C₇ cycloalkyl optionally substituted with one or more hydroxy, and R¹ is C₁-C₆ alkyl;
R² is 3- to 7-membered heterocycloalkyl optionally substituted with one or more hydroxy, and R¹ is C₁-C₆ alkyl;
R² is 3- to 7-membered heterocycloalkyl optionally substituted with one or more hydroxy, and R¹ is halo;
R² is C₁-C₆ alkyl optionally substituted with one or more NR⁸R⁹, and R¹ is C₁-C₆ alkyl;
R² is C₁-C₆ alkyl optionally substituted with one or more NR⁸R⁹, and R¹ is halo;

R² is C₁-C₆ alkyl optionally substituted with one or more oxo, and R¹ is methyl; or

R² is C₁-C₆ alkyl optionally substituted with one or more C₁-C₆ alkoxy, and R¹ is C₁-C₆ alkyl.

In some embodiments, of the compound of formula AA,

the substituted ring A is

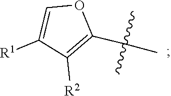

and R¹ and R² are one of the following combinations:
R¹ is 1-hydroxy-2-methylpropan-2-yl, and R² is methyl;
R¹ is 2-hydroxy-2-propyl and R² is methyl;
R¹ is 2-hydroxy-2-propyl and R² is isopropyl;
R¹ is 2-hydroxy-2-propyl and R² is 2-hydroxy-2-propyl;
R¹ is 2-hydroxy-2-propyl and R² is 1-hydroxyethyl;
R¹ is hydroxymethyl and R² is methyl;
R¹ is 1-hydroxyethyl and R² is methyl;
R¹ is 2-hydroxyethyl and R² is methyl;
R¹ is 1-hydroxy-2-propyl and R² is methyl;
R¹ is 2-hydroxy-2-propyl and R² is phenyl;
R¹ is 2-hydroxy-2-propyl and R² is pyridyl;
R¹ is 2-hydroxy-2-propyl and R² is pyrazolyl;
R¹ is 2-hydroxy-2-propyl, and R² is S(O₂)CH₃;
R¹ is 2-hydroxy-2-propyl and R² is chloro;
R¹ is 2-hydroxy-2-propyl and R² is fluoro;
R¹ is 1-hydroxy-1-cyclopropyl, and R² is methyl;
R¹ is 1-hydroxy-1-cyclobutyl, and R² is methyl;
R¹ is 1-hydroxy-1-cyclopentyl, and R² is methyl;
R¹ is 1-hydroxy-1-cyclohexyl, and R² is methyl;
R¹ is morpholinyl, and R² is methyl;
R¹ is 1,3-dioxolan-2-yl, and R² is methyl;
R¹ is 1,3-dioxolan-2-yl, and R² is fluoro;
R¹ is 1,3-dioxolan-2-yl, and R² is chloro;
R¹ is COCH₃, and R² is methyl;
R¹ is 2-methoxy-2-propyl, and R² is methyl;
R¹ is (dimethylamino)methyl, and R² is methyl.
R² is 1-hydroxy-2-methylpropan-2-yl, and R¹ is methyl;
R² is 2-hydroxy-2-propyl and R¹ is methyl;
R² is 2-hydroxy-2-propyl and R¹ is isopropyl;
R² is 2-hydroxy-2-propyl and R¹ is 1-hydroxyethyl;
R² is hydroxymethyl and R¹ is methyl;
R² is 1-hydroxyethyl and R¹ is methyl;
R² is 2-hydroxyethyl and R¹ is methyl;
R² is 1-hydroxy-2-propyl and R¹ is methyl;
R² is 2-hydroxy-2-propyl and R¹ is phenyl;
R² is C₁-C₆ alkyl optionally substituted with one or more hydroxy, and R¹ is 5- to 10-membered heteroaryl;
R² is 2-hydroxy-2-propyl and R¹ is pyridyl;
R² is 2-hydroxy-2-propyl and R¹ is pyrazolyl;
R² is C₁-C₆ alkyl optionally substituted with one or more hydroxy, and R¹ is S(O₂)CH₃;
R² is 2-hydroxy-2-propyl and R¹ is chloro;
R² is 2-hydroxy-2-propyl and R¹ is fluoro;
R² is C₃-C₇ cycloalkyl optionally substituted with one or more hydroxy, and R¹ is C₁-C₆ alkyl;
R² is 1-hydroxy-1-cyclopropyl, and R¹ is methyl;
R² is 1-hydroxy-1-cyclobutyl, and R¹ is methyl;
R² is 1-hydroxy-1-cyclopentyl, and R¹ is methyl;
R² is 1-hydroxy-1-cyclohexyl, and R¹ is methyl;
R² is morpholinyl, and R¹ is methyl;
R² is 1,3-dioxolan-2-yl, and R¹ is methyl;
R² is 1,3-dioxolan-2-yl, and R¹ is fluoro;
R² is 1,3-dioxolan-2-yl, and R¹ is chloro;
R² is C₁-C₆ alkyl optionally substituted with one or more oxo, and R¹ is methyl;
R² is (dimethylamino)methyl, and R¹ is methyl;
R² is COCH₃, and R¹ is methyl; or
R² is 2-methoxy-2-propyl, and R¹ is methyl.

In some embodiments, of the compound of formula AA,

the substituted ring A is

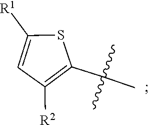

and R¹ and R² are one of the following combinations:
R¹ is C₁-C₆ alkyl optionally substituted with one or more hydroxy, and R² is C₁-C₆ alkyl optionally substituted with one or more hydroxy;
R¹ is C₁-C₆ alkyl optionally substituted with one or more hydroxy, and R² is C₆-C₁₀ aryl;
R¹ is C₁-C₆ alkyl optionally substituted with one or more hydroxy, and R² is 5- to 10-membered heteroaryl;
R¹ is C₁-C₆ alkyl optionally substituted with one or more hydroxy, and R² is SF₅;
R¹ is C₁-C₆ alkyl optionally substituted with one or more hydroxy, and R² is S(O₂)C₁-C₆ alkyl;
R¹ is C₁-C₆ alkyl optionally substituted with one or more hydroxy, and R² is halo;
R¹ is C₃-C₇ cycloalkyl optionally substituted with one or more hydroxy, and R² is C₁-C₆ alkyl;
R¹ is 3- to 7-membered heterocycloalkyl optionally substituted with one or more hydroxy, and R² is C₁-C₆ alkyl;
R¹ is 3- to 7-membered heterocycloalkyl optionally substituted with one or more hydroxy, and R² is halo;
R¹ is C₁-C₆ alkyl optionally substituted with one or more oxo, and R² is methyl;
R¹ is C₁-C₆ alkyl optionally substituted with one or more C₁-C₆ alkoxy, and R² is C₁-C₆ alkyl;
R¹ is C₁-C₆ alkyl optionally substituted with one or more NR⁸R⁹, and R² is C₁-C₆ alkyl;
R¹ is C₁-C₆ alkyl optionally substituted with one or more NR⁸R⁹, and R² is halo;
R² is C₁-C₆ alkyl optionally substituted with one or more hydroxy, and R¹ is C₆-C₁₀ aryl;
R² is C₁-C₆ alkyl optionally substituted with one or more hydroxy, and R¹ is 5- to 10-membered heteroaryl;
R² is C₁-C₆ alkyl optionally substituted with one or more hydroxy, and R¹ is SF₅.
R² is C₁-C₆ alkyl optionally substituted with one or more hydroxy, and R¹ is S(O₂)C₁-C₆ alkyl;
R² is C₁-C₆ alkyl optionally substituted with one or more hydroxy, and R¹ is halo;
R² is C₃-C₇ cycloalkyl optionally substituted with one or more hydroxy, and R¹ is C₁-C₆ alkyl;
R² is 3- to 7-membered heterocycloalkyl optionally substituted with one or more hydroxy, and R¹ is C₁-C₆ alkyl;
R² is 3- to 7-membered heterocycloalkyl optionally substituted with one or more hydroxy, and R¹ is halo;
R² is C₁-C₆ alkyl optionally substituted with one or more NR⁸R⁹, and R¹ is C₁-C₆ alkyl;
R² is C₁-C₆ alkyl optionally substituted with one or more NR⁸R⁹, and R¹ is halo;
R² is C₁-C₆ alkyl optionally substituted with one or more oxo, and R¹ is methyl; or
R² is C₁-C₆ alkyl optionally substituted with one or more C₁-C₆ alkoxy, and R¹ is C₁-C₆ alkyl.

In some embodiments, of the compound of formula AA,

the substituted ring A is

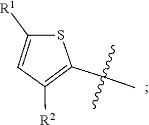

and R¹ and R² are one of the following combinations:
R¹ is 1-hydroxy-2-methylpropan-2-yl, and R² is methyl;
R¹ is 2-hydroxy-2-propyl and R² is methyl;
R¹ is 2-hydroxy-2-propyl and R² is isopropyl;
R¹ is 2-hydroxy-2-propyl and R² is 2-hydroxy-2-propyl;
R¹ is 2-hydroxy-2-propyl and R² is 1-hydroxyethyl;
R¹ is hydroxymethyl and R² is methyl;
R¹ is 1-hydroxyethyl and R² is methyl;
R¹ is 2-hydroxyethyl and R² is methyl;
R¹ is 1-hydroxy-2-propyl and R² is methyl;
R¹ is 2-hydroxy-2-propyl and R² is phenyl;
R¹ is 2-hydroxy-2-propyl and R² is pyridyl;
R¹ is 2-hydroxy-2-propyl and R² is pyrazolyl;
R¹ is 2-hydroxy-2-propyl, and R² is S(O₂)CH₃;
R¹ is 2-hydroxy-2-propyl and R² is chloro;
R¹ is 2-hydroxy-2-propyl and R² is fluoro;
R¹ is 1-hydroxy-1-cyclopropyl, and R² is methyl;
R¹ is 1-hydroxy-1-cyclobutyl, and R² is methyl;
R¹ is 1-hydroxy-1-cyclopentyl, and R² is methyl;
R¹ is 1-hydroxy-1-cyclohexyl, and R² is methyl;
R¹ is morpholinyl, and R² is methyl;
R¹ is 1,3-dioxolan-2-yl, and R² is methyl;
R¹ is 1,3-dioxolan-2-yl, and R² is fluoro;
R¹ is 1,3-dioxolan-2-yl, and R² is chloro;
R¹ is COCH₃, and R² is methyl;
R¹ is 2-methoxy-2-propyl, and R² is methyl;
R¹ is (dimethylamino)methyl, and R² is methyl.
R² is 1-hydroxy-2-methylpropan-2-yl, and R¹ is methyl;
R² is 2-hydroxy-2-propyl and R¹ is methyl;
R² is 2-hydroxy-2-propyl and R¹ is isopropyl;
R² is 2-hydroxy-2-propyl and R¹ is 1-hydroxyethyl;
R² is hydroxymethyl and R¹ is methyl;
R² is 1-hydroxyethyl and R¹ is methyl;
R² is 2-hydroxyethyl and R¹ is methyl;
R² is 1-hydroxy-2-propyl and R¹ is methyl;
R² is 2-hydroxy-2-propyl and R¹ is phenyl;
R² is C₁-C₆ alkyl optionally substituted with one or more hydroxy, and R¹ is 5- to 10-membered heteroaryl;
R² is 2-hydroxy-2-propyl and R¹ is pyridyl;
R² is 2-hydroxy-2-propyl and R¹ is pyrazolyl;
R² is C₁-C₆ alkyl optionally substituted with one or more hydroxy, and R¹ is S(O₂)CH₃;
R² is 2-hydroxy-2-propyl and R¹ is chloro;
R² is 2-hydroxy-2-propyl and R¹ is fluoro;
R² is C₃-C₇ cycloalkyl optionally substituted with one or more hydroxy, and R¹ is C₁-C₆ alkyl;
R² is 1-hydroxy-1-cyclopropyl, and R¹ is methyl;
R² is 1-hydroxy-1-cyclobutyl, and R¹ is methyl;
R² is 1-hydroxy-1-cyclopentyl, and R¹ is methyl;
R² is 1-hydroxy-1-cyclohexyl, and R¹ is methyl;
R² is morpholinyl, and R¹ is methyl;
R² is 1,3-dioxolan-2-yl, and R¹ is methyl;
R² is 1,3-dioxolan-2-yl, and R¹ is fluoro;
R² is 1,3-dioxolan-2-yl, and R¹ is chloro;
R² is C₁-C₆ alkyl optionally substituted with one or more oxo, and R¹ is methyl;
R² is (dimethylamino)methyl, and R¹ is methyl;
R² is COCH₃, and R¹ is methyl; or
R² is 2-methoxy-2-propyl, and R¹ is methyl.

In some embodiments, of the compound of formula AA,

the substituted ring A is

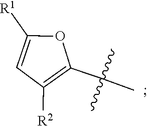

and R¹ and R² are one of the following combinations:
R¹ is C₁-C₆ alkyl optionally substituted with one or more hydroxy, and R² is C₁-C₆ alkyl optionally substituted with one or more hydroxy;
R¹ is C₁-C₆ alkyl optionally substituted with one or more hydroxy, and R² is C₆-C₁₀ aryl;
R¹ is C₁-C₆ alkyl optionally substituted with one or more hydroxy, and R² is 5- to 10-membered heteroaryl;
R¹ is C₁-C₆ alkyl optionally substituted with one or more hydroxy, and R² is SF₅;
R¹ is C₁-C₆ alkyl optionally substituted with one or more hydroxy, and R² is S(O₂)C₁-C₆ alkyl;
R¹ is C₁-C₆ alkyl optionally substituted with one or more hydroxy, and R² is halo;
R¹ is C₃-C₇ cycloalkyl optionally substituted with one or more hydroxy, and R² is C₁-C₆ alkyl;
R¹ is 3- to 7-membered heterocycloalkyl optionally substituted with one or more hydroxy, and R² is C₁-C₆ alkyl;
R¹ is 3- to 7-membered heterocycloalkyl optionally substituted with one or more hydroxy, and R² is halo;
R¹ is C₁-C₆ alkyl optionally substituted with one or more oxo, and R² is methyl;
R¹ is C₁-C₆ alkyl optionally substituted with one or more C₁-C₆ alkoxy, and R² is C₁-C₆ alkyl;
R¹ is C₁-C₆ alkyl optionally substituted with one or more NR⁸R⁹, and R² is C₁-C₆ alkyl;
R¹ is C₁-C₆ alkyl optionally substituted with one or more NR⁸R⁹, and R² is halo;
R² is C₁-C₆ alkyl optionally substituted with one or more hydroxy, and R¹ is C₆-C₁₀ aryl;
R² is C₁-C₆ alkyl optionally substituted with one or more hydroxy, and R¹ is 5- to 10-membered heteroaryl;
R² is C₁-C₆ alkyl optionally substituted with one or more hydroxy, and R¹ is SF₅.
R² is C₁-C₆ alkyl optionally substituted with one or more hydroxy, and R¹ is S(O₂)C₁-C₆ alkyl;
R² is C₁-C₆ alkyl optionally substituted with one or more hydroxy, and R¹ is halo;
R² is C₃-C₇ cycloalkyl optionally substituted with one or more hydroxy, and R¹ is C₁-C₆ alkyl;
R² is 3- to 7-membered heterocycloalkyl optionally substituted with one or more hydroxy, and R¹ is C₁-C₆ alkyl;
R² is 3- to 7-membered heterocycloalkyl optionally substituted with one or more hydroxy, and R¹ is halo;
R² is C₁-C₆ alkyl optionally substituted with one or more NR⁸R⁹, and R¹ is C₁-C₆ alkyl;
R² is C₁-C₆ alkyl optionally substituted with one or more NR⁸R⁹, and R¹ is halo;
R² is C₁-C₆ alkyl optionally substituted with one or more oxo, and R¹ is methyl; or
R² is C₁-C₆ alkyl optionally substituted with one or more C₁-C₆ alkoxy, and R¹ is C₁-C₆ alkyl.

In some embodiments, of the compound of formula AA,

the substituted ring A is

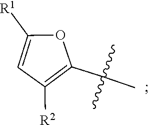

and R¹ and R² are one of the following combinations:
R¹ is 1-hydroxy-2-methylpropan-2-yl, and R² is methyl;
R¹ is 2-hydroxy-2-propyl and R² is methyl;
R¹ is 2-hydroxy-2-propyl and R² is isopropyl;
R¹ is 2-hydroxy-2-propyl and R² is 2-hydroxy-2-propyl;
R¹ is 2-hydroxy-2-propyl and R² is 1-hydroxyethyl;
R¹ is hydroxymethyl and R² is methyl;
R¹ is 1-hydroxyethyl and R² is methyl;
R¹ is 2-hydroxyethyl and R² is methyl;
R¹ is 1-hydroxy-2-propyl and R² is methyl;
R¹ is 2-hydroxy-2-propyl and R² is phenyl;
R¹ is 2-hydroxy-2-propyl and R² is pyridyl;
R¹ is 2-hydroxy-2-propyl and R² is pyrazolyl;
R¹ is 2-hydroxy-2-propyl, and R² is S(O₂)CH₃;
R¹ is 2-hydroxy-2-propyl and R² is chloro;
R¹ is 2-hydroxy-2-propyl and R² is fluoro;
R¹ is 1-hydroxy-1-cyclopropyl, and R² is methyl;
R¹ is 1-hydroxy-1-cyclobutyl, and R² is methyl;
R¹ is 1-hydroxy-1-cyclopentyl, and R² is methyl;
R¹ is 1-hydroxy-1-cyclohexyl, and R² is methyl;
R¹ is morpholinyl, and R² is methyl;
R¹ is 1,3-dioxolan-2-yl, and R² is methyl;
R¹ is 1,3-dioxolan-2-yl, and R² is fluoro;
R¹ is 1,3-dioxolan-2-yl, and R² is chloro;
R¹ is COCH₃, and R² is methyl;
R¹ is 2-methoxy-2-propyl, and R² is methyl;
R¹ is (dimethylamino)methyl, and R² is methyl.
R² is 1-hydroxy-2-methylpropan-2-yl, and R¹ is methyl;
R² is 2-hydroxy-2-propyl and R¹ is methyl;
R² is 2-hydroxy-2-propyl and R¹ is isopropyl;
R² is 2-hydroxy-2-propyl and R¹ is 1-hydroxyethyl;
R² is hydroxymethyl and R¹ is methyl;
R² is 1-hydroxyethyl and R¹ is methyl;
R² is 2-hydroxyethyl and R¹ is methyl;
R² is 1-hydroxy-2-propyl and R¹ is methyl;
R² is 2-hydroxy-2-propyl and R¹ is phenyl;
R² is C₁-C₆ alkyl optionally substituted with one or more hydroxy, and R¹ is 5- to 10-membered heteroaryl;
R² is 2-hydroxy-2-propyl and R¹ is pyridyl;
R² is 2-hydroxy-2-propyl and R¹ is pyrazolyl;
R² is C₁-C₆ alkyl optionally substituted with one or more hydroxy, and R¹ is S(O₂)CH₃;
R² is 2-hydroxy-2-propyl and R¹ is chloro;
R² is 2-hydroxy-2-propyl and R¹ is fluoro;
R² is C₃-C₇ cycloalkyl optionally substituted with one or more hydroxy, and R¹ is C₁-C₆ alkyl;
R² is 1-hydroxy-1-cyclopropyl, and R¹ is methyl;
R² is 1-hydroxy-1-cyclobutyl, and R¹ is methyl;
R² is 1-hydroxy-1-cyclopentyl, and R¹ is methyl;
R² is 1-hydroxy-1-cyclohexyl, and R¹ is methyl;
R² is morpholinyl, and R¹ is methyl;
R² is 1,3-dioxolan-2-yl, and R¹ is methyl;
R² is 1,3-dioxolan-2-yl, and R¹ is fluoro;
R² is 1,3-dioxolan-2-yl, and R¹ is chloro;
R² is C₁-C₆ alkyl optionally substituted with one or more oxo, and R¹ is methyl;
R² is (dimethylamino)methyl, and R¹ is methyl;
R² is COCH₃, and R¹ is methyl; or
R² is 2-methoxy-2-propyl, and R¹ is methyl.

In some embodiments, of the compound of formula AA,

the substituted ring A is

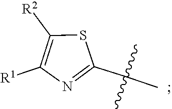

and R¹ and R² are one of the following combinations:
R¹ is C₁-C₆ alkyl optionally substituted with one or more hydroxy, and R² is C₁-C₆ alkyl optionally substituted with one or more hydroxy;
R¹ is C₁-C₆ alkyl optionally substituted with one or more hydroxy, and R² is C₆-C₁₀ aryl;
R¹ is C₁-C₆ alkyl optionally substituted with one or more hydroxy, and R² is 5- to 10-membered heteroaryl;
R¹ is C₁-C₆ alkyl optionally substituted with one or more hydroxy, and R² is SF₅;
R¹ is C₁-C₆ alkyl optionally substituted with one or more hydroxy, and R² is S(O₂)C₁-C₆ alkyl;
R¹ is C₁-C₆ alkyl optionally substituted with one or more hydroxy, and R² is halo;
R¹ is C₃-C₇ cycloalkyl optionally substituted with one or more hydroxy, and R² is C₁-C₆ alkyl;
R¹ is 3- to 7-membered heterocycloalkyl optionally substituted with one or more hydroxy, and R² is C₁-C₆ alkyl;
R¹ is 3- to 7-membered heterocycloalkyl optionally substituted with one or more hydroxy, and R² is halo;
R¹ is C₁-C₆ alkyl optionally substituted with one or more oxo, and R² is methyl;
R¹ is C₁-C₆ alkyl optionally substituted with one or more C₁-C₆ alkoxy, and R² is C₁-C₆ alkyl;
R¹ is C₁-C₆ alkyl optionally substituted with one or more NR⁸R⁹, and R² is C₁-C₆ alkyl;
R¹ is C₁-C₆ alkyl optionally substituted with one or more NR⁸R⁹, and R² is halo;
R² is C₁-C₆ alkyl optionally substituted with one or more hydroxy, and R¹ is C₆-C₁₀ aryl;
R² is C₁-C₆ alkyl optionally substituted with one or more hydroxy, and R¹ is 5- to 10-membered heteroaryl;
R² is C₁-C₆ alkyl optionally substituted with one or more hydroxy, and R¹ is SF₅.
R² is C₁-C₆ alkyl optionally substituted with one or more hydroxy, and R¹ is S(O₂)C₁-C₆ alkyl;
R² is C₁-C₆ alkyl optionally substituted with one or more hydroxy, and R¹ is halo;
R² is C₃-C₇ cycloalkyl optionally substituted with one or more hydroxy, and R¹ is C₁-C₆ alkyl;
R² is 3- to 7-membered heterocycloalkyl optionally substituted with one or more hydroxy, and R¹ is C₁-C₆ alkyl;
R² is 3- to 7-membered heterocycloalkyl optionally substituted with one or more hydroxy, and R¹ is halo;
R² is C₁-C₆ alkyl optionally substituted with one or more NR⁸R⁹, and R¹ is C₁-C₆ alkyl;
R² is C₁-C₆ alkyl optionally substituted with one or more NR⁸R⁹, and R¹ is halo;
R² is C₁-C₆ alkyl optionally substituted with one or more oxo, and R¹ is methyl; or
R² is C₁-C₆ alkyl optionally substituted with one or more C₁-C₆ alkoxy, and R¹ is C₁-C₆ alkyl.

In some embodiments, of the compound of formula AA,

the substituted ring A is

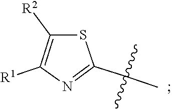

and R¹ and R² are one of the following combinations:
R¹ is 1-hydroxy-2-methylpropan-2-yl, and R² is methyl;
R¹ is 2-hydroxy-2-propyl and R² is methyl;
R¹ is 2-hydroxy-2-propyl and R² is isopropyl;
R¹ is 2-hydroxy-2-propyl and R² is 2-hydroxy-2-propyl;
R¹ is 2-hydroxy-2-propyl and R² is 1-hydroxyethyl;
R¹ is hydroxymethyl and R² is methyl;
R¹ is 1-hydroxyethyl and R² is methyl;
R¹ is 2-hydroxyethyl and R² is methyl;
R¹ is 1-hydroxy-2-propyl and R² is methyl;
R¹ is 2-hydroxy-2-propyl and R² is phenyl;
R¹ is 2-hydroxy-2-propyl and R² is pyridyl;
R¹ is 2-hydroxy-2-propyl and R² is pyrazolyl;
R¹ is 2-hydroxy-2-propyl, and R² is S(O₂)CH₃;
R¹ is 2-hydroxy-2-propyl and R² is chloro;
R¹ is 2-hydroxy-2-propyl and R² is fluoro;
R¹ is 1-hydroxy-1-cyclopropyl, and R² is methyl;
R¹ is 1-hydroxy-1-cyclobutyl, and R² is methyl;
R¹ is 1-hydroxy-1-cyclopentyl, and R² is methyl;
R¹ is 1-hydroxy-1-cyclohexyl, and R² is methyl;
R¹ is morpholinyl, and R² is methyl;
R¹ is 1,3-dioxolan-2-yl, and R² is methyl;
R¹ is 1,3-dioxolan-2-yl, and R² is fluoro;
R¹ is 1,3-dioxolan-2-yl, and R² is chloro;
R¹ is COCH₃, and R² is methyl;
R¹ is 2-methoxy-2-propyl, and R² is methyl;
R¹ is (dimethylamino)methyl, and R² is methyl.
R² is 1-hydroxy-2-methylpropan-2-yl, and R¹ is methyl;
R² is 2-hydroxy-2-propyl and R¹ is methyl;
R² is 2-hydroxy-2-propyl and R¹ is isopropyl;
R² is 2-hydroxy-2-propyl and R¹ is 1-hydroxyethyl;
R² is hydroxymethyl and R¹ is methyl;
R² is 1-hydroxyethyl and R¹ is methyl;
R² is 2-hydroxyethyl and R¹ is methyl;
R² is 1-hydroxy-2-propyl and R¹ is methyl;
R² is 2-hydroxy-2-propyl and R¹ is phenyl;
R² is C₁-C₆ alkyl optionally substituted with one or more hydroxy, and R¹ is 5- to 10-membered heteroaryl;
R² is 2-hydroxy-2-propyl and R¹ is pyridyl;
R² is 2-hydroxy-2-propyl and R¹ is pyrazolyl;
R² is C₁-C₆ alkyl optionally substituted with one or more hydroxy, and R¹ is S(O₂)CH₃;
R² is 2-hydroxy-2-propyl and R¹ is chloro;
R² is 2-hydroxy-2-propyl and R¹ is fluoro;
R² is C₃-C₇ cycloalkyl optionally substituted with one or more hydroxy, and R¹ is C₁-C₆ alkyl;
R² is 1-hydroxy-1-cyclopropyl, and R¹ is methyl;
R² is 1-hydroxy-1-cyclobutyl, and R¹ is methyl;
R² is 1-hydroxy-1-cyclopentyl, and R¹ is methyl;
R² is 1-hydroxy-1-cyclohexyl, and R¹ is methyl;
R² is morpholinyl, and R¹ is methyl;
R² is 1,3-dioxolan-2-yl, and R¹ is methyl;
R² is 1,3-dioxolan-2-yl, and R¹ is fluoro;
R² is 1,3-dioxolan-2-yl, and R¹ is chloro;
R² is C₁-C₆ alkyl optionally substituted with one or more oxo, and R¹ is methyl;
R² is (dimethylamino)methyl, and R¹ is methyl;
R² is COCH₃, and R¹ is methyl; or
R² is 2-methoxy-2-propyl, and R¹ is methyl.

In some embodiments, of the compound of formula AA,

the substituted ring A is

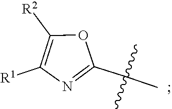

and R¹ and R² are one of the following combinations:
R¹ is C₁-C₆ alkyl optionally substituted with one or more hydroxy, and R² is C₁-C₆ alkyl optionally substituted with one or more hydroxy;
R¹ is C₁-C₆ alkyl optionally substituted with one or more hydroxy, and R² is C₆-C₁₀ aryl;
R¹ is C₁-C₆ alkyl optionally substituted with one or more hydroxy, and R² is 5- to 10-membered heteroaryl;
R¹ is C₁-C₆ alkyl optionally substituted with one or more hydroxy, and R² is SF₅;
R¹ is C₁-C₆ alkyl optionally substituted with one or more hydroxy, and R² is S(O₂)C₁-C₆ alkyl;
R¹ is C₁-C₆ alkyl optionally substituted with one or more hydroxy, and R² is halo;
R¹ is C₃-C₇ cycloalkyl optionally substituted with one or more hydroxy, and R² is C₁-C₆ alkyl;
R¹ is 3- to 7-membered heterocycloalkyl optionally substituted with one or more hydroxy, and R² is C₁-C₆ alkyl;
R¹ is 3- to 7-membered heterocycloalkyl optionally substituted with one or more hydroxy, and R² is halo;
R¹ is C₁-C₆ alkyl optionally substituted with one or more oxo, and R² is methyl;
R¹ is C₁-C₆ alkyl optionally substituted with one or more C₁-C₆ alkoxy, and R² is C₁-C₆ alkyl;
R¹ is C₁-C₆ alkyl optionally substituted with one or more NR⁸R⁹, and R² is C₁-C₆ alkyl;
R¹ is C₁-C₆ alkyl optionally substituted with one or more NR⁸R⁹, and R² is halo;
R² is C₁-C₆ alkyl optionally substituted with one or more hydroxy, and R¹ is C₆-C₁₀ aryl;
R² is C₁-C₆ alkyl optionally substituted with one or more hydroxy, and R¹ is 5- to 10-membered heteroaryl;
R² is C₁-C₆ alkyl optionally substituted with one or more hydroxy, and R¹ is SF₅.
R² is C₁-C₆ alkyl optionally substituted with one or more hydroxy, and R¹ is S(O₂)C₁-C₆ alkyl;
R² is C₁-C₆ alkyl optionally substituted with one or more hydroxy, and R¹ is halo;
R² is C₃-C₇ cycloalkyl optionally substituted with one or more hydroxy, and R¹ is C₁-C₆ alkyl;
R² is 3- to 7-membered heterocycloalkyl optionally substituted with one or more hydroxy, and R¹ is C₁-C₆ alkyl;
R² is 3- to 7-membered heterocycloalkyl optionally substituted with one or more hydroxy, and R¹ is halo;
R² is C₁-C₆ alkyl optionally substituted with one or more NR⁸R⁹, and R¹ is C₁-C₆ alkyl;
R² is C₁-C₆ alkyl optionally substituted with one or more NR⁸R⁹, and R¹ is halo;
R² is C₁-C₆ alkyl optionally substituted with one or more oxo, and R¹ is methyl; or
R² is C₁-C₆ alkyl optionally substituted with one or more C₁-C₆ alkoxy, and R¹ is C₁-C₆ alkyl.

In some embodiments, of the compound of formula AA,

the substituted ring A is

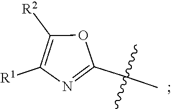

and R¹ and R² are one of the following combinations:
R¹ is 1-hydroxy-2-methylpropan-2-yl, and R² is methyl;
R¹ is 2-hydroxy-2-propyl and R² is methyl;
R¹ is 2-hydroxy-2-propyl and R² is isopropyl;
R¹ is 2-hydroxy-2-propyl and R² is 2-hydroxy-2-propyl;
R¹ is 2-hydroxy-2-propyl and R² is 1-hydroxyethyl;
R¹ is hydroxymethyl and R² is methyl;
R¹ is 1-hydroxyethyl and R² is methyl;
R¹ is 2-hydroxyethyl and R² is methyl;
R¹ is 1-hydroxy-2-propyl and R² is methyl;
R¹ is 2-hydroxy-2-propyl and R² is phenyl;
R¹ is 2-hydroxy-2-propyl and R² is pyridyl;
R¹ is 2-hydroxy-2-propyl and R² is pyrazolyl;
R¹ is 2-hydroxy-2-propyl, and R² is S(O₂)CH₃;
R¹ is 2-hydroxy-2-propyl and R² is chloro;
R¹ is 2-hydroxy-2-propyl and R² is fluoro;
R¹ is 1-hydroxy-1-cyclopropyl, and R² is methyl;
R¹ is 1-hydroxy-1-cyclobutyl, and R² is methyl;
R¹ is 1-hydroxy-1-cyclopentyl, and R² is methyl;
R¹ is 1-hydroxy-1-cyclohexyl, and R² is methyl;
R¹ is morpholinyl, and R² is methyl;
R¹ is 1,3-dioxolan-2-yl, and R² is methyl;
R¹ is 1,3-dioxolan-2-yl, and R² is fluoro;
R¹ is 1,3-dioxolan-2-yl, and R² is chloro;
R¹ is COCH₃, and R² is methyl;
R¹ is 2-methoxy-2-propyl, and R² is methyl;
R¹ is (dimethylamino)methyl, and R² is methyl.
R² is 1-hydroxy-2-methylpropan-2-yl, and R¹ is methyl;
R² is 2-hydroxy-2-propyl and R¹ is methyl;
R² is 2-hydroxy-2-propyl and R¹ is isopropyl;
R² is 2-hydroxy-2-propyl and R¹ is 1-hydroxyethyl;
R² is hydroxymethyl and R¹ is methyl;
R² is 1-hydroxyethyl and R¹ is methyl;
R² is 2-hydroxyethyl and R¹ is methyl;
R² is 1-hydroxy-2-propyl and R¹ is methyl;
R² is 2-hydroxy-2-propyl and R¹ is phenyl;
R² is C₁-C₆ alkyl optionally substituted with one or more hydroxy, and R¹ is 5- to 10-membered heteroaryl;
R² is 2-hydroxy-2-propyl and R¹ is pyridyl;
R² is 2-hydroxy-2-propyl and R¹ is pyrazolyl;
R² is C₁-C₆ alkyl optionally substituted with one or more hydroxy, and R¹ is S(O₂)CH₃;
R² is 2-hydroxy-2-propyl and R¹ is chloro;
R² is 2-hydroxy-2-propyl and R¹ is fluoro;
R² is C₃-C₇ cycloalkyl optionally substituted with one or more hydroxy, and R¹ is C₁-C₆ alkyl;
R² is 1-hydroxy-1-cyclopropyl, and R¹ is methyl;
R² is 1-hydroxy-1-cyclobutyl, and R¹ is methyl;
R² is 1-hydroxy-1-cyclopentyl, and R¹ is methyl;
R² is 1-hydroxy-1-cyclohexyl, and R¹ is methyl;
R² is morpholinyl, and R¹ is methyl;
R² is 1,3-dioxolan-2-yl, and R¹ is methyl;
R² is 1,3-dioxolan-2-yl, and R¹ is fluoro;
R² is 1,3-dioxolan-2-yl, and R¹ is chloro;
R² is C₁-C₆ alkyl optionally substituted with one or more oxo, and R¹ is methyl;
R² is (dimethylamino)methyl, and R¹ is methyl;
R² is COCH₃, and R¹ is methyl; or
R² is 2-methoxy-2-propyl, and R¹ is methyl.

In some embodiments, of the compound of formula AA,

the substituted ring A is

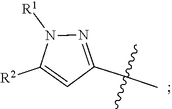

and R¹ and R² are one of the following combinations:
R¹ is 1-hydroxy-2-methylpropan-2-yl, and R² is methyl;
R¹ is 2-hydroxy-2-propyl and R² is methyl;
R¹ is 2-hydroxy-2-propyl and R² is isopropyl;
R¹ is 2-hydroxy-2-propyl and R² is 2-hydroxy-2-propyl;
R¹ is 2-hydroxy-2-propyl and R² is 1-hydroxyethyl;
R¹ is hydroxymethyl and R² is methyl;
R¹ is 1-hydroxyethyl and R² is methyl;
R¹ is 2-hydroxyethyl and R² is methyl;
R¹ is 1-hydroxy-2-propyl and R² is methyl;
R¹ is 2-hydroxy-2-propyl and R² is phenyl;
R¹ is 2-hydroxy-2-propyl and R² is pyridyl;
R¹ is 2-hydroxy-2-propyl and R² is pyrazolyl;
R¹ is 2-hydroxy-2-propyl, and R² is S(O₂)CH₃;
R¹ is 2-hydroxy-2-propyl and R² is chloro;
R¹ is 2-hydroxy-2-propyl and R² is fluoro;
R¹ is 1-hydroxy-1-cyclopropyl, and R² is methyl;
R¹ is 1-hydroxy-1-cyclobutyl, and R² is methyl;
R¹ is 1-hydroxy-1-cyclopentyl, and R² is methyl;
R¹ is 1-hydroxy-1-cyclohexyl, and R² is methyl;
R¹ is morpholinyl, and R² is methyl;
R¹ is 1,3-dioxolan-2-yl, and R² is methyl;
R¹ is 1,3-dioxolan-2-yl, and R² is fluoro;
R¹ is 1,3-dioxolan-2-yl, and R² is chloro;
R¹ is COCH₃, and R² is methyl;
R¹ is 2-methoxy-2-propyl, and R² is methyl;
R¹ is (dimethylamino)methyl, and R² is methyl.
R² is 1-hydroxy-2-methylpropan-2-yl, and R¹ is methyl;
R² is 2-hydroxy-2-propyl and R¹ is methyl;
R² is 2-hydroxy-2-propyl and R¹ is isopropyl;
R² is 2-hydroxy-2-propyl and R¹ is 1-hydroxyethyl;
R² is hydroxymethyl and R¹ is methyl;
R² is 1-hydroxyethyl and R¹ is methyl;
R² is 2-hydroxyethyl and R¹ is methyl;
R² is 1-hydroxy-2-propyl and R¹ is methyl;
R² is 2-hydroxy-2-propyl and R¹ is phenyl;
R² is C₁-C₆ alkyl optionally substituted with one or more hydroxy, and R¹ is 5- to 10-membered heteroaryl;
R² is 2-hydroxy-2-propyl and R¹ is pyridyl;
R² is 2-hydroxy-2-propyl and R¹ is pyrazolyl;
R² is C₁-C₆ alkyl optionally substituted with one or more hydroxy, and R¹ is S(O₂)CH₃;
R² is 2-hydroxy-2-propyl and R¹ is chloro;
R² is 2-hydroxy-2-propyl and R¹ is fluoro;
R² is C₃-C₇ cycloalkyl optionally substituted with one or more hydroxy, and R¹ is C₁-C₆ alkyl;
R² is 1-hydroxy-1-cyclopropyl, and R¹ is methyl;
R² is 1-hydroxy-1-cyclobutyl, and R¹ is methyl;
R² is 1-hydroxy-1-cyclopentyl, and R¹ is methyl;
R² is 1-hydroxy-1-cyclohexyl, and R¹ is methyl;
R² is morpholinyl, and R¹ is methyl;
R² is 1,3-dioxolan-2-yl, and R¹ is methyl;
R² is 1,3-dioxolan-2-yl, and R¹ is fluoro;
R² is 1,3-dioxolan-2-yl, and R¹ is chloro;
R² is C₁-C₆ alkyl optionally substituted with one or more oxo, and R¹ is methyl;
R² is (dimethylamino)methyl, and R¹ is methyl;
R² is COCH₃, and R¹ is methyl; or
R² is 2-methoxy-2-propyl, and R¹ is methyl.

In some embodiments, of the compound of formula AA,

the substituted ring A is

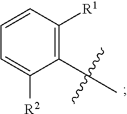

and R¹ and R² are one of the following combinations:
R¹ is C₁-C₆ alkyl optionally substituted with one or more hydroxy, and R² is C₁-C₆ alkyl optionally substituted with one or more hydroxy;
R¹ is C₁-C₆ alkyl optionally substituted with one or more hydroxy, and R² is C₆-C₁₀ aryl;
R¹ is C₁-C₆ alkyl optionally substituted with one or more hydroxy, and R² is 5- to 10-membered heteroaryl;
R¹ is C₁-C₆ alkyl optionally substituted with one or more hydroxy, and R² is SF₅;
R¹ is C₁-C₆ alkyl optionally substituted with one or more hydroxy, and R² is S(O₂)C₁-C₆ alkyl;
R¹ is C₁-C₆ alkyl optionally substituted with one or more hydroxy, and R² is halo;
R¹ is C₃-C₇ cycloalkyl optionally substituted with one or more hydroxy, and R² is C₁-C₆ alkyl;
R¹ is 3- to 7-membered heterocycloalkyl optionally substituted with one or more hydroxy, and R² is C₁-C₆ alkyl;
R¹ is 3- to 7-membered heterocycloalkyl optionally substituted with one or more hydroxy, and R² is halo;
R¹ is C₁-C₆ alkyl optionally substituted with one or more oxo, and R² is methyl;
R¹ is C₁-C₆ alkyl optionally substituted with one or more C₁-C₆ alkoxy, and R² is C₁-C₆ alkyl;
R¹ is C₁-C₆ alkyl optionally substituted with one or more NR⁸R⁹, and R² is C₁-C₆ alkyl;
R¹ is C₁-C₆ alkyl optionally substituted with one or more NR⁸R⁹, and R² is halo;
R² is C₁-C₆ alkyl optionally substituted with one or more hydroxy, and R¹ is C₆-C₁₀ aryl;
R² is C₁-C₆ alkyl optionally substituted with one or more hydroxy, and R¹ is 5- to 10-membered heteroaryl;
R² is C₁-C₆ alkyl optionally substituted with one or more hydroxy, and R¹ is SF₅.
R² is C₁-C₆ alkyl optionally substituted with one or more hydroxy, and R¹ is S(O₂)C₁-C₆ alkyl;
R² is C₁-C₆ alkyl optionally substituted with one or more hydroxy, and R¹ is halo;
R² is C₃-C₇ cycloalkyl optionally substituted with one or more hydroxy, and R¹ is C₁-C₆ alkyl;
R² is 3- to 7-membered heterocycloalkyl optionally substituted with one or more hydroxy, and R¹ is C₁-C₆ alkyl;
R² is 3- to 7-membered heterocycloalkyl optionally substituted with one or more hydroxy, and R¹ is halo;
R² is C₁-C₆ alkyl optionally substituted with one or more NR⁸R⁹, and R¹ is C₁-C₆ alkyl;
R² is C₁-C₆ alkyl optionally substituted with one or more NR⁸R⁹, and R¹ is halo;
R² is C₁-C₆ alkyl optionally substituted with one or more oxo, and R¹ is methyl; or
R² is C₁-C₆ alkyl optionally substituted with one or more C₁-C₆ alkoxy, and R¹ is C₁-C₆ alkyl.

In some embodiments, of the compound of formula AA,

the substituted ring A is

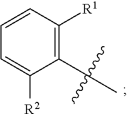

and R¹ and R² are one of the following combinations:
R¹ is 1-hydroxy-2-methylpropan-2-yl, and R² is methyl;
R¹ is 2-hydroxy-2-propyl and R² is methyl;
R¹ is 2-hydroxy-2-propyl and R² is isopropyl;
R¹ is 2-hydroxy-2-propyl and R² is 2-hydroxy-2-propyl;
R¹ is 2-hydroxy-2-propyl and R² is 1-hydroxyethyl;
R¹ is hydroxymethyl and R² is methyl;
R¹ is 1-hydroxyethyl and R² is methyl;
R¹ is 2-hydroxyethyl and R² is methyl;
R¹ is 1-hydroxy-2-propyl and R² is methyl;
R¹ is 2-hydroxy-2-propyl and R² is phenyl;
R¹ is 2-hydroxy-2-propyl and R² is pyridyl;
R¹ is 2-hydroxy-2-propyl and R² is pyrazolyl;
R¹ is 2-hydroxy-2-propyl, and R² is S(O₂)CH₃;
R¹ is 2-hydroxy-2-propyl and R² is chloro;
R¹ is 2-hydroxy-2-propyl and R² is fluoro;
R¹ is 1-hydroxy-1-cyclopropyl, and R² is methyl;
R¹ is 1-hydroxy-1-cyclobutyl, and R² is methyl;
R¹ is 1-hydroxy-1-cyclopentyl, and R² is methyl;
R¹ is 1-hydroxy-1-cyclohexyl, and R² is methyl;
R¹ is morpholinyl, and R² is methyl;
R¹ is 1,3-dioxolan-2-yl, and R² is methyl;
R¹ is 1,3-dioxolan-2-yl, and R² is fluoro;
R¹ is 1,3-dioxolan-2-yl, and R² is chloro;
R¹ is COCH₃, and R² is methyl;
R¹ is 2-methoxy-2-propyl, and R² is methyl;
R¹ is (dimethylamino)methyl, and R² is methyl.
R² is 1-hydroxy-2-methylpropan-2-yl, and R¹ is methyl;
R² is 2-hydroxy-2-propyl and R¹ is methyl;
R² is 2-hydroxy-2-propyl and R¹ is isopropyl;
R² is 2-hydroxy-2-propyl and R¹ is 1-hydroxyethyl;
R² is hydroxymethyl and R¹ is methyl;
R² is 1-hydroxyethyl and R¹ is methyl;
R² is 2-hydroxyethyl and R¹ is methyl;
R² is 1-hydroxy-2-propyl and R¹ is methyl;
R² is 2-hydroxy-2-propyl and R¹ is phenyl;
R² is C₁-C₆ alkyl optionally substituted with one or more hydroxy, and R¹ is 5- to 10-membered heteroaryl;
R² is 2-hydroxy-2-propyl and R¹ is pyridyl;
R² is 2-hydroxy-2-propyl and R¹ is pyrazolyl;
R² is C₁-C₆ alkyl optionally substituted with one or more hydroxy, and R¹ is S(O₂)CH₃;
R² is 2-hydroxy-2-propyl and R¹ is chloro;
R² is 2-hydroxy-2-propyl and R¹ is fluoro;
R² is C₃-C₇ cycloalkyl optionally substituted with one or more hydroxy, and R¹ is C₁-C₆ alkyl;
R² is 1-hydroxy-1-cyclopropyl, and R¹ is methyl;
R² is 1-hydroxy-1-cyclobutyl, and R¹ is methyl;
R² is 1-hydroxy-1-cyclopentyl, and R¹ is methyl;
R² is 1-hydroxy-1-cyclohexyl, and R¹ is methyl;
R² is morpholinyl, and R¹ is methyl;
R² is 1,3-dioxolan-2-yl, and R¹ is methyl;
R² is 1,3-dioxolan-2-yl, and R¹ is fluoro;
R² is 1,3-dioxolan-2-yl, and R¹ is chloro;
R² is C₁-C₆ alkyl optionally substituted with one or more oxo, and R¹ is methyl;
R² is (dimethylamino)methyl, and R¹ is methyl;
R² is COCH₃, and R¹ is methyl; or
R² is 2-methoxy-2-propyl, and R¹ is methyl.

In some embodiments, of the compound of formula AA,

the substituted ring A is

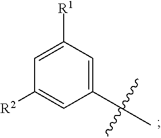

and R¹ and R² are one of the following combinations:
R¹ is C₁-C₆ alkyl optionally substituted with one or more hydroxy, and R² is C₁-C₆ alkyl optionally substituted with one or more hydroxy;
R¹ is C₁-C₆ alkyl optionally substituted with one or more hydroxy, and R² is C₆-C₁₀ aryl;
R¹ is C₁-C₆ alkyl optionally substituted with one or more hydroxy, and R² is 5- to 10-membered heteroaryl;
R¹ is C₁-C₆ alkyl optionally substituted with one or more hydroxy, and R² is SF₅;
R¹ is C₁-C₆ alkyl optionally substituted with one or more hydroxy, and R² is S(O₂)C₁-C₆ alkyl;
R¹ is C₁-C₆ alkyl optionally substituted with one or more hydroxy, and R² is halo;
R¹ is C₃-C₇ cycloalkyl optionally substituted with one or more hydroxy, and R² is C₁-C₆ alkyl;
R¹ is 3- to 7-membered heterocycloalkyl optionally substituted with one or more hydroxy, and R² is C₁-C₆ alkyl;
R¹ is 3- to 7-membered heterocycloalkyl optionally substituted with one or more hydroxy, and R² is halo;
R¹ is C₁-C₆ alkyl optionally substituted with one or more oxo, and R² is methyl;
R¹ is C₁-C₆ alkyl optionally substituted with one or more C₁-C₆ alkoxy, and R² is C₁-C₆ alkyl;
R¹ is C₁-C₆ alkyl optionally substituted with one or more NR⁸R⁹, and R² is C₁-C₆ alkyl;
R¹ is C₁-C₆ alkyl optionally substituted with one or more NR⁸R⁹, and R² is halo;
R² is C₁-C₆ alkyl optionally substituted with one or more hydroxy, and R¹ is C₆-C₁₀ aryl;
R² is C₁-C₆ alkyl optionally substituted with one or more hydroxy, and R¹ is 5- to 10-membered heteroaryl;
R² is C₁-C₆ alkyl optionally substituted with one or more hydroxy, and R¹ is SF₅.
R² is C₁-C₆ alkyl optionally substituted with one or more hydroxy, and R¹ is S(O₂)C₁-C₆ alkyl;
R² is C₁-C₆ alkyl optionally substituted with one or more hydroxy, and R¹ is halo;
R² is C₃-C₇ cycloalkyl optionally substituted with one or more hydroxy, and R¹ is C₁-C₆ alkyl;
R² is 3- to 7-membered heterocycloalkyl optionally substituted with one or more hydroxy, and R¹ is C₁-C₆ alkyl;
R² is 3- to 7-membered heterocycloalkyl optionally substituted with one or more hydroxy, and R¹ is halo;
R² is C₁-C₆ alkyl optionally substituted with one or more NR⁸R⁹, and R¹ is C₁-C₆ alkyl;
R² is C₁-C₆ alkyl optionally substituted with one or more NR⁸R⁹, and R¹ is halo;
R² is C₁-C₆ alkyl optionally substituted with one or more oxo, and R¹ is methyl; or
R² is C₁-C₆ alkyl optionally substituted with one or more C₁-C₆ alkoxy, and R¹ is C₁-C₆ alkyl.

In some embodiments, of the compound of formula AA,

the substituted ring A is

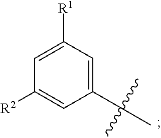

and R¹ and R² are one of the following combinations:
R¹ is 1-hydroxy-2-methylpropan-2-yl, and R² is methyl;
R¹ is 2-hydroxy-2-propyl and R² is methyl;
R¹ is 2-hydroxy-2-propyl and R² is isopropyl;
R¹ is 2-hydroxy-2-propyl and R² is 2-hydroxy-2-propyl;
R¹ is 2-hydroxy-2-propyl and R² is 1-hydroxyethyl;
R¹ is hydroxymethyl and R² is methyl;
R¹ is 1-hydroxyethyl and R² is methyl;
R¹ is 2-hydroxyethyl and R² is methyl;
R¹ is 1-hydroxy-2-propyl and R² is methyl;
R¹ is 2-hydroxy-2-propyl and R² is phenyl;
R¹ is 2-hydroxy-2-propyl and R² is pyridyl;
R¹ is 2-hydroxy-2-propyl and R² is pyrazolyl;
R¹ is 2-hydroxy-2-propyl, and R² is S(O₂)CH₃;
R¹ is 2-hydroxy-2-propyl and R² is chloro;
R¹ is 2-hydroxy-2-propyl and R² is fluoro;
R¹ is 1-hydroxy-1-cyclopropyl, and R² is methyl;
R¹ is 1-hydroxy-1-cyclobutyl, and R² is methyl;
R¹ is 1-hydroxy-1-cyclopentyl, and R² is methyl;
R¹ is 1-hydroxy-1-cyclohexyl, and R² is methyl;
R¹ is morpholinyl, and R² is methyl;
R¹ is 1,3-dioxolan-2-yl, and R² is methyl;
R¹ is 1,3-dioxolan-2-yl, and R² is fluoro;
R¹ is 1,3-dioxolan-2-yl, and R² is chloro;
R¹ is COCH₃, and R² is methyl;
R¹ is 2-methoxy-2-propyl, and R² is methyl;
R¹ is (dimethylamino)methyl, and R² is methyl.
R² is 1-hydroxy-2-methylpropan-2-yl, and R¹ is methyl;
R² is 2-hydroxy-2-propyl and R¹ is methyl;
R² is 2-hydroxy-2-propyl and R¹ is isopropyl;
R² is 2-hydroxy-2-propyl and R¹ is 1-hydroxyethyl;
R² is hydroxymethyl and R¹ is methyl;
R² is 1-hydroxyethyl and R¹ is methyl;
R² is 2-hydroxyethyl and R¹ is methyl;
R² is 1-hydroxy-2-propyl and R¹ is methyl;
R² is 2-hydroxy-2-propyl and R¹ is phenyl;
R² is C₁-C₆ alkyl optionally substituted with one or more hydroxy, and R¹ is 5- to 10-membered heteroaryl;
R² is 2-hydroxy-2-propyl and R¹ is pyridyl;
R² is 2-hydroxy-2-propyl and R¹ is pyrazolyl;
R² is C₁-C₆ alkyl optionally substituted with one or more hydroxy, and R¹ is S(O₂)CH₃;
R² is 2-hydroxy-2-propyl and R¹ is chloro;
R² is 2-hydroxy-2-propyl and R¹ is fluoro;
R² is C₃-C₇ cycloalkyl optionally substituted with one or more hydroxy, and R¹ is C₁-C₆ alkyl;
R² is 1-hydroxy-1-cyclopropyl, and R¹ is methyl;
R² is 1-hydroxy-1-cyclobutyl, and R¹ is methyl;
R² is 1-hydroxy-1-cyclopentyl, and R¹ is methyl;
R² is 1-hydroxy-1-cyclohexyl, and R¹ is methyl;
R² is morpholinyl, and R¹ is methyl;
R² is 1,3-dioxolan-2-yl, and R¹ is methyl;
R² is 1,3-dioxolan-2-yl, and R¹ is fluoro;
R² is 1,3-dioxolan-2-yl, and R¹ is chloro;
R² is C₁-C₆ alkyl optionally substituted with one or more oxo, and R¹ is methyl;
R² is (dimethylamino)methyl, and R¹ is methyl;
R² is COCH₃, and R¹ is methyl; or
R² is 2-methoxy-2-propyl, and R¹ is methyl.

In some embodiments, of the compound of formula AA,

the substituted ring A is

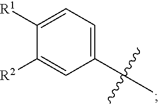

and R¹ and R² are one of the following combinations:
R¹ is C₁-C₆ alkyl optionally substituted with one or more hydroxy, and R² is C₁-C₆ alkyl optionally substituted with one or more hydroxy;
R¹ is C₁-C₆ alkyl optionally substituted with one or more hydroxy, and R² is C₆-C₁₀ aryl;
R¹ is C₁-C₆ alkyl optionally substituted with one or more hydroxy, and R² is 5- to 10-membered heteroaryl;
R¹ is C₁-C₆ alkyl optionally substituted with one or more hydroxy, and R² is SF₅;
R¹ is C₁-C₆ alkyl optionally substituted with one or more hydroxy, and R² is S(O₂)C₁-C₆ alkyl;
R¹ is C₁-C₆ alkyl optionally substituted with one or more hydroxy, and R² is halo;
R¹ is C₃-C₇ cycloalkyl optionally substituted with one or more hydroxy, and R² is C₁-C₆ alkyl;
R¹ is 3- to 7-membered heterocycloalkyl optionally substituted with one or more hydroxy, and R² is C₁-C₆ alkyl;
R¹ is 3- to 7-membered heterocycloalkyl optionally substituted with one or more hydroxy, and R² is halo;
R¹ is C₁-C₆ alkyl optionally substituted with one or more oxo, and R² is methyl;
R¹ is C₁-C₆ alkyl optionally substituted with one or more C₁-C₆ alkoxy, and R² is C₁-C₆ alkyl;
R¹ is C₁-C₆ alkyl optionally substituted with one or more NR⁸R⁹, and R² is C₁-C₆ alkyl;
R¹ is C₁-C₆ alkyl optionally substituted with one or more NR⁸R⁹, and R² is halo;
R² is C₁-C₆ alkyl optionally substituted with one or more hydroxy, and R¹ is C₆-C₁₀ aryl;
R² is C₁-C₆ alkyl optionally substituted with one or more hydroxy, and R¹ is 5- to 10-membered heteroaryl;
R² is C₁-C₆ alkyl optionally substituted with one or more hydroxy, and R¹ is SF₅.
R² is C₁-C₆ alkyl optionally substituted with one or more hydroxy, and R¹ is S(O₂)C₁-C₆ alkyl;
R² is C₁-C₆ alkyl optionally substituted with one or more hydroxy, and R¹ is halo;
R² is C₃-C₇ cycloalkyl optionally substituted with one or more hydroxy, and R¹ is C₁-C₆ alkyl;
R² is 3- to 7-membered heterocycloalkyl optionally substituted with one or more hydroxy, and R¹ is C₁-C₆ alkyl;
R² is 3- to 7-membered heterocycloalkyl optionally substituted with one or more hydroxy, and R¹ is halo;
R² is C₁-C₆ alkyl optionally substituted with one or more NR⁸R⁹, and R¹ is C₁-C₆ alkyl;
R² is C₁-C₆ alkyl optionally substituted with one or more NR⁸R⁹, and R¹ is halo;
R² is C₁-C₆ alkyl optionally substituted with one or more oxo, and R¹ is methyl; or
R² is C₁-C₆ alkyl optionally substituted with one or more C₁-C₆ alkoxy, and R¹ is C₁-C₆ alkyl.

In some embodiments, of the compound of formula AA,

the substituted ring A is

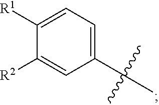

and R¹ and R² are one of the following combinations:
R¹ is 1-hydroxy-2-methylpropan-2-yl, and R² is methyl;
R¹ is 2-hydroxy-2-propyl and R² is methyl;
R¹ is 2-hydroxy-2-propyl and R² is isopropyl;
R¹ is 2-hydroxy-2-propyl and R² is 2-hydroxy-2-propyl;
R¹ is 2-hydroxy-2-propyl and R² is 1-hydroxyethyl;
R¹ is hydroxymethyl and R² is methyl;
R¹ is 1-hydroxyethyl and R² is methyl;
R¹ is 2-hydroxyethyl and R² is methyl;
R¹ is 1-hydroxy-2-propyl and R² is methyl;
R¹ is 2-hydroxy-2-propyl and R² is phenyl;
R¹ is 2-hydroxy-2-propyl and R² is pyridyl;
R¹ is 2-hydroxy-2-propyl and R² is pyrazolyl;
R¹ is 2-hydroxy-2-propyl, and R² is S(O₂)CH₃;
R¹ is 2-hydroxy-2-propyl and R² is chloro;
R¹ is 2-hydroxy-2-propyl and R² is fluoro;
R¹ is 1-hydroxy-1-cyclopropyl, and R² is methyl;
R¹ is 1-hydroxy-1-cyclobutyl, and R² is methyl;
R¹ is 1-hydroxy-1-cyclopentyl, and R² is methyl;
R¹ is 1-hydroxy-1-cyclohexyl, and R² is methyl;
R¹ is morpholinyl, and R² is methyl;
R¹ is 1,3-dioxolan-2-yl, and R² is methyl;
R¹ is 1,3-dioxolan-2-yl, and R² is fluoro;
R¹ is 1,3-dioxolan-2-yl, and R² is chloro;
R¹ is COCH₃, and R² is methyl;
R¹ is 2-methoxy-2-propyl, and R² is methyl;
R¹ is (dimethylamino)methyl, and R² is methyl.
R² is 1-hydroxy-2-methylpropan-2-yl, and R¹ is methyl;
R² is 2-hydroxy-2-propyl and R¹ is methyl;
R² is 2-hydroxy-2-propyl and R¹ is isopropyl;
R² is 2-hydroxy-2-propyl and R¹ is 1-hydroxyethyl;
R² is hydroxymethyl and R¹ is methyl;
R² is 1-hydroxyethyl and R¹ is methyl;
R² is 2-hydroxyethyl and R¹ is methyl;
R² is 1-hydroxy-2-propyl and R¹ is methyl;
R² is 2-hydroxy-2-propyl and R¹ is phenyl;
R² is C₁-C₆ alkyl optionally substituted with one or more hydroxy, and R¹ is 5- to 10-membered heteroaryl;
R² is 2-hydroxy-2-propyl and R¹ is pyridyl;
R² is 2-hydroxy-2-propyl and R¹ is pyrazolyl;
R² is C₁-C₆ alkyl optionally substituted with one or more hydroxy, and R¹ is S(O₂)CH₃;
R² is 2-hydroxy-2-propyl and R¹ is chloro;
R² is 2-hydroxy-2-propyl and R¹ is fluoro;
R² is C₃-C₇ cycloalkyl optionally substituted with one or more hydroxy, and R¹ is C₁-C₆ alkyl;
R² is 1-hydroxy-1-cyclopropyl, and R¹ is methyl;
R² is 1-hydroxy-1-cyclobutyl, and R¹ is methyl;
R² is 1-hydroxy-1-cyclopentyl, and R¹ is methyl;
R² is 1-hydroxy-1-cyclohexyl, and R¹ is methyl;
R² is morpholinyl, and R¹ is methyl;
R² is 1,3-dioxolan-2-yl, and R¹ is methyl;
R² is 1,3-dioxolan-2-yl, and R¹ is fluoro;
R² is 1,3-dioxolan-2-yl, and R¹ is chloro;
R² is C₁-C₆ alkyl optionally substituted with one or more oxo, and R¹ is methyl;
R² is (dimethylamino)methyl, and R¹ is methyl;
R² is COCH₃, and R¹ is methyl; or
R² is 2-methoxy-2-propyl, and R¹ is methyl.

In some embodiments, of the compound of formula AA,

the substituted ring A is

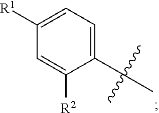

and R¹ and R² are one of the following combinations:
R¹ is C₁-C₆ alkyl optionally substituted with one or more hydroxy, and R² is C₁-C₆ alkyl optionally substituted with one or more hydroxy;
R¹ is C₁-C₆ alkyl optionally substituted with one or more hydroxy, and R² is C₆-C₁₀ aryl;
R¹ is C₁-C₆ alkyl optionally substituted with one or more hydroxy, and R² is 5- to 10-membered heteroaryl;
R¹ is C₁-C₆ alkyl optionally substituted with one or more hydroxy, and R² is SF₅;
R¹ is C₁-C₆ alkyl optionally substituted with one or more hydroxy, and R² is S(O₂)C₁-C₆ alkyl;
R¹ is C₁-C₆ alkyl optionally substituted with one or more hydroxy, and R² is halo;
R¹ is C₃-C₇ cycloalkyl optionally substituted with one or more hydroxy, and R² is C₁-C₆ alkyl;
R¹ is 3- to 7-membered heterocycloalkyl optionally substituted with one or more hydroxy, and R² is C₁-C₆ alkyl;
R¹ is 3- to 7-membered heterocycloalkyl optionally substituted with one or more hydroxy, and R² is halo;
R¹ is C₁-C₆ alkyl optionally substituted with one or more oxo, and R² is methyl;
R¹ is C₁-C₆ alkyl optionally substituted with one or more C₁-C₆ alkoxy, and R² is C₁-C₆ alkyl;
R¹ is C₁-C₆ alkyl optionally substituted with one or more NR⁸R⁹, and R² is C₁-C₆ alkyl;
R¹ is C₁-C₆ alkyl optionally substituted with one or more NR⁸R⁹, and R² is halo;
R² is C₁-C₆ alkyl optionally substituted with one or more hydroxy, and R¹ is C₆-C₁₀ aryl;
R² is C₁-C₆ alkyl optionally substituted with one or more hydroxy, and R¹ is 5- to 10-membered heteroaryl;
R² is C₁-C₆ alkyl optionally substituted with one or more hydroxy, and R¹ is SF₅.
R² is C₁-C₆ alkyl optionally substituted with one or more hydroxy, and R¹ is S(O₂)C₁-C₆ alkyl;
R² is C₁-C₆ alkyl optionally substituted with one or more hydroxy, and R¹ is halo;
R² is C₃-C₇ cycloalkyl optionally substituted with one or more hydroxy, and R¹ is C₁-C₆ alkyl;
R² is 3- to 7-membered heterocycloalkyl optionally substituted with one or more hydroxy, and R¹ is C₁-C₆ alkyl;
R² is 3- to 7-membered heterocycloalkyl optionally substituted with one or more hydroxy, and R¹ is halo;
R² is C₁-C₆ alkyl optionally substituted with one or more NR⁸R⁹, and R¹ is C₁-C₆ alkyl;
R² is C₁-C₆ alkyl optionally substituted with one or more NR⁸R⁹, and R¹ is halo;
R² is C₁-C₆ alkyl optionally substituted with one or more oxo, and R¹ is methyl; or
R² is C₁-C₆ alkyl optionally substituted with one or more C₁-C₆ alkoxy, and R¹ is C₁-C₆ alkyl.

In some embodiments, of the compound of formula AA,

the substituted ring A is

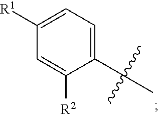

and R¹ and R² are one of the following combinations:
R¹ is 1-hydroxy-2-methylpropan-2-yl, and R² is methyl;
R¹ is 2-hydroxy-2-propyl and R² is methyl;
R¹ is 2-hydroxy-2-propyl and R² is isopropyl;
R¹ is 2-hydroxy-2-propyl and R² is 2-hydroxy-2-propyl;
R¹ is 2-hydroxy-2-propyl and R² is 1-hydroxyethyl;
R¹ is hydroxymethyl and R² is methyl;
R¹ is 1-hydroxyethyl and R² is methyl;
R¹ is 2-hydroxyethyl and R² is methyl;
R¹ is 1-hydroxy-2-propyl and R² is methyl;
R¹ is 2-hydroxy-2-propyl and R² is phenyl;
R¹ is 2-hydroxy-2-propyl and R² is pyridyl;
R¹ is 2-hydroxy-2-propyl and R² is pyrazolyl;
R¹ is 2-hydroxy-2-propyl, and R² is S(O₂)CH₃;
R¹ is 2-hydroxy-2-propyl and R² is chloro;
R¹ is 2-hydroxy-2-propyl and R² is fluoro;
R¹ is 1-hydroxy-1-cyclopropyl, and R² is methyl;
R¹ is 1-hydroxy-1-cyclobutyl, and R² is methyl;
R¹ is 1-hydroxy-1-cyclopentyl, and R² is methyl;
R¹ is 1-hydroxy-1-cyclohexyl, and R² is methyl;
R¹ is morpholinyl, and R² is methyl;
R¹ is 1,3-dioxolan-2-yl, and R² is methyl;
R¹ is 1,3-dioxolan-2-yl, and R² is fluoro;
R¹ is 1,3-dioxolan-2-yl, and R² is chloro;
R¹ is COCH₃, and R² is methyl;
R¹ is 2-methoxy-2-propyl, and R² is methyl;
R¹ is (dimethylamino)methyl, and R² is methyl.
R² is 1-hydroxy-2-methylpropan-2-yl, and R¹ is methyl;
R² is 2-hydroxy-2-propyl and R¹ is methyl;
R² is 2-hydroxy-2-propyl and R¹ is isopropyl;
R² is 2-hydroxy-2-propyl and R¹ is 1-hydroxyethyl;
R² is hydroxymethyl and R¹ is methyl;
R² is 1-hydroxyethyl and R¹ is methyl;
R² is 2-hydroxyethyl and R¹ is methyl;
R² is 1-hydroxy-2-propyl and R¹ is methyl;
R² is 2-hydroxy-2-propyl and R¹ is phenyl;
R² is C₁-C₆ alkyl optionally substituted with one or more hydroxy, and R¹ is 5- to 10-membered heteroaryl;
R² is 2-hydroxy-2-propyl and R¹ is pyridyl;
R² is 2-hydroxy-2-propyl and R¹ is pyrazolyl;
R² is C₁-C₆ alkyl optionally substituted with one or more hydroxy, and R¹ is S(O₂)CH₃;
R² is 2-hydroxy-2-propyl and R¹ is chloro;
R² is 2-hydroxy-2-propyl and R¹ is fluoro;
R² is C₃-C₇ cycloalkyl optionally substituted with one or more hydroxy, and R¹ is C₁-C₆ alkyl;
R² is 1-hydroxy-1-cyclopropyl, and R¹ is methyl;
R² is 1-hydroxy-1-cyclobutyl, and R¹ is methyl;
R² is 1-hydroxy-1-cyclopentyl, and R¹ is methyl;
R² is 1-hydroxy-1-cyclohexyl, and R¹ is methyl;
R² is morpholinyl, and R¹ is methyl;
R² is 1,3-dioxolan-2-yl, and R¹ is methyl;
R² is 1,3-dioxolan-2-yl, and R¹ is fluoro;
R² is 1,3-dioxolan-2-yl, and R¹ is chloro;
R² is (dimethylamino)methyl, and R¹ is methyl;
R² is C₁-C₆ alkyl optionally substituted with one or more oxo, and R¹ is methyl; or
R² is COCH₃, and R¹ is methyl.

In some embodiments, of the compound of formula AA,

the substituted ring A is

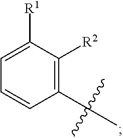

and R¹ and R² are one of the following combinations:
R¹ is C₁-C₆ alkyl optionally substituted with one or more hydroxy, and R² is C₁-C₆ alkyl optionally substituted with one or more hydroxy;
R¹ is C₁-C₆ alkyl optionally substituted with one or more hydroxy, and R² is C₆-C₁₀ aryl;
R¹ is C₁-C₆ alkyl optionally substituted with one or more hydroxy, and R² is 5- to 10-membered heteroaryl;
R¹ is C₁-C₆ alkyl optionally substituted with one or more hydroxy, and R² is SF₅;
R¹ is C₁-C₆ alkyl optionally substituted with one or more hydroxy, and R² is S(O₂)C₁-C₆ alkyl;
R¹ is C₁-C₆ alkyl optionally substituted with one or more hydroxy, and R² is halo;
R¹ is C₃-C₇ cycloalkyl optionally substituted with one or more hydroxy, and R² is C₁-C₆ alkyl;
R¹ is 3- to 7-membered heterocycloalkyl optionally substituted with one or more hydroxy, and R² is C₁-C₆ alkyl;
R¹ is 3- to 7-membered heterocycloalkyl optionally substituted with one or more hydroxy, and R² is halo;
R¹ is C₁-C₆ alkyl optionally substituted with one or more oxo, and R² is methyl;
R¹ is C₁-C₆ alkyl optionally substituted with one or more C₁-C₆ alkoxy, and R² is C₁-C₆ alkyl;
R¹ is C₁-C₆ alkyl optionally substituted with one or more NR⁸R⁹, and R² is C₁-C₆ alkyl;
R¹ is C₁-C₆ alkyl optionally substituted with one or more NR⁸R⁹, and R² is halo;
R² is C₁-C₆ alkyl optionally substituted with one or more hydroxy, and R¹ is C₆-C₁₀ aryl;
R² is C₁-C₆ alkyl optionally substituted with one or more hydroxy, and R¹ is 5- to 10-membered heteroaryl;
R² is C₁-C₆ alkyl optionally substituted with one or more hydroxy, and R¹ is SF₅.
R² is C₁-C₆ alkyl optionally substituted with one or more hydroxy, and R¹ is S(O₂)C₁-C₆ alkyl;
R² is C₁-C₆ alkyl optionally substituted with one or more hydroxy, and R¹ is halo;
R² is C₃-C₇ cycloalkyl optionally substituted with one or more hydroxy, and R¹ is C₁-C₆ alkyl;
R² is 3- to 7-membered heterocycloalkyl optionally substituted with one or more hydroxy, and R¹ is C₁-C₆ alkyl;
R² is 3- to 7-membered heterocycloalkyl optionally substituted with one or more hydroxy, and R¹ is halo;
R² is C₁-C₆ alkyl optionally substituted with one or more NR⁸R⁹, and R¹ is C₁-C₆ alkyl;
R² is C₁-C₆ alkyl optionally substituted with one or more NR⁸R⁹, and R¹ is halo;
R² is C₁-C₆ alkyl optionally substituted with one or more oxo, and R¹ is methyl; or
R² is C₁-C₆ alkyl optionally substituted with one or more C₁-C₆ alkoxy, and R¹ is C₁-C₆ alkyl.

In some embodiments, of the compound of formula AA,

the substituted ring A is

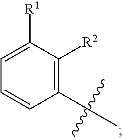

and R¹ and R² are one of the following combinations:
R¹ is 1-hydroxy-2-methylpropan-2-yl, and R² is methyl;
R¹ is 2-hydroxy-2-propyl and R² is methyl;
R¹ is 2-hydroxy-2-propyl and R² is isopropyl;
R¹ is 2-hydroxy-2-propyl and R² is 2-hydroxy-2-propyl;
R¹ is 2-hydroxy-2-propyl and R² is 1-hydroxyethyl;
R¹ is hydroxymethyl and R² is methyl;
R¹ is 1-hydroxyethyl and R² is methyl;
R¹ is 2-hydroxyethyl and R² is methyl;
R¹ is 1-hydroxy-2-propyl and R² is methyl;
R¹ is 2-hydroxy-2-propyl and R² is phenyl;
R¹ is 2-hydroxy-2-propyl and R² is pyridyl;
R¹ is 2-hydroxy-2-propyl and R² is pyrazolyl;
R¹ is 2-hydroxy-2-propyl, and R² is S(O₂)CH₃;
R¹ is 2-hydroxy-2-propyl and R² is chloro;
R¹ is 2-hydroxy-2-propyl and R² is fluoro;
R¹ is 1-hydroxy-1-cyclopropyl, and R² is methyl;
R¹ is 1-hydroxy-1-cyclobutyl, and R² is methyl;
R¹ is 1-hydroxy-1-cyclopentyl, and R² is methyl;
R¹ is 1-hydroxy-1-cyclohexyl, and R² is methyl;
R¹ is morpholinyl, and R² is methyl;
R¹ is 1,3-dioxolan-2-yl, and R² is methyl;
R¹ is 1,3-dioxolan-2-yl, and R² is fluoro;
R¹ is 1,3-dioxolan-2-yl, and R² is chloro;
R¹ is COCH₃, and R² is methyl;
R¹ is 2-methoxy-2-propyl, and R² is methyl;
R¹ is C₁-C₆ alkyl optionally substituted with one or more NR⁸R⁹, and R² is C₁-C₆ alkyl;
R¹ is C₁-C₆ alkyl optionally substituted with one or more NR⁸R⁹, and R² is halo;
R² is 1-hydroxy-2-methylpropan-2-yl, and R¹ is methyl;
R² is 2-hydroxy-2-propyl and R¹ is methyl;
R² is 2-hydroxy-2-propyl and R¹ is isopropyl;
R² is 2-hydroxy-2-propyl and R¹ is 1-hydroxyethyl;
R² is hydroxymethyl and R¹ is methyl;
R² is 1-hydroxyethyl and R¹ is methyl;
R² is 2-hydroxyethyl and R¹ is methyl;
R² is 1-hydroxy-2-propyl and R¹ is methyl;
R² is 2-hydroxy-2-propyl and R¹ is phenyl;
R² is C₁-C₆ alkyl optionally substituted with one or more hydroxy, and R¹ is 5- to 10-membered heteroaryl;
R² is 2-hydroxy-2-propyl and R¹ is pyridyl;
R² is 2-hydroxy-2-propyl and R¹ is pyrazolyl;
R² is C₁-C₆ alkyl optionally substituted with one or more hydroxy, and R¹ is S(O₂)CH₃;
R² is 2-hydroxy-2-propyl and R¹ is chloro;
R² is 2-hydroxy-2-propyl and R¹ is fluoro;
R² is C₃-C₇ cycloalkyl optionally substituted with one or more hydroxy, and R¹ is C₁-C₆ alkyl;
R² is 1-hydroxy-1-cyclopropyl, and R¹ is methyl;
R² is 1-hydroxy-1-cyclobutyl, and R¹ is methyl;
R² is 1-hydroxy-1-cyclopentyl, and R¹ is methyl;
R² is 1-hydroxy-1-cyclohexyl, and R¹ is methyl;
R² is morpholinyl, and R¹ is methyl;
R² is 1,3-dioxolan-2-yl, and R¹ is methyl;
R² is 1,3-dioxolan-2-yl, and R¹ is fluoro;
R² is 1,3-dioxolan-2-yl, and R¹ is chloro;
R² is C₁-C₆ alkyl optionally substituted with one or more oxo, and R¹ is methyl;
R² is C₁-C₆ alkyl optionally substituted with one or more NR⁸R⁹, and R¹ is C₁-C₆ alkyl;
R² is C₁-C₆ alkyl optionally substituted with one or more NR⁸R⁹, and R¹ is halo;
R² is COCH₃, and R¹ is methyl; or
R² is 2-methoxy-2-propyl, and R¹ is methyl.

In some embodiments, of the compound of formula AA,

the substituted ring A is

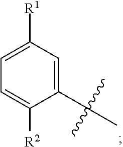

and R¹ and R² are one of the following combinations:
R¹ is C₁-C₆ alkyl optionally substituted with one or more hydroxy, and R² is C₁-C₆ alkyl optionally substituted with one or more hydroxy;
R¹ is R¹ is C₁-C₆ alkyl optionally substituted with one or more hydroxy, and R² is C₆-C₁₀ aryl;
R¹ is R¹ is C₁-C₆ alkyl optionally substituted with one or more hydroxy, and R² is 5- to 10-membered heteroaryl;
R¹ is C₁-C₆ alkyl optionally substituted with one or more hydroxy, and R² is SF₅;
R¹ is R¹ is C₁-C₆ alkyl optionally substituted with one or more hydroxy, and R² is S(O₂)C₁-C₆ alkyl;
R¹ is R¹ is C₁-C₆ alkyl optionally substituted with one or more hydroxy, and R² is halo;
R¹ is R¹ is C₃-C₇ cycloalkyl optionally substituted with one or more hydroxy, and R² is C₁-C₆ alkyl;
R¹ is 3- to 7-membered heterocycloalkyl optionally substituted with one or more hydroxy, and R² is C₁-C₆ alkyl;
R¹ is 3- to 7-membered heterocycloalkyl optionally substituted with one or more hydroxy, and R² is halo;
R¹ is C₁-C₆ alkyl optionally substituted with one or more oxo, and R² is methyl;
R¹ is C₁-C₆ alkyl optionally substituted with one or more C₁-C₆ alkoxy, and R² is C₁-C₆ alkyl;
R¹ is C₁-C₆ alkyl optionally substituted with one or more NR⁸R⁹, and R² is C₁-C₆ alkyl;
R¹ is C₁-C₆ alkyl optionally substituted with one or more NR⁸R⁹, and R² is halo;
R² is C₁-C₆ alkyl optionally substituted with one or more hydroxy, and R¹ is C₆-C₁₀ aryl;
R² is C₁-C₆ alkyl optionally substituted with one or more hydroxy, and R¹ is 5- to 10-membered heteroaryl;
R² is C₁-C₆ alkyl optionally substituted with one or more hydroxy, and R¹ is SF₅.
R² is C₁-C₆ alkyl optionally substituted with one or more hydroxy, and R¹ is S(O₂)C₁-C₆ alkyl;
R² is C₁-C₆ alkyl optionally substituted with one or more hydroxy, and R¹ is halo;
R² is C₃-C₇ cycloalkyl optionally substituted with one or more hydroxy, and R¹ is C₁-C₆ alkyl;
R² is 3- to 7-membered heterocycloalkyl optionally substituted with one or more hydroxy, and R¹ is C₁-C₆ alkyl;
R² is 3- to 7-membered heterocycloalkyl optionally substituted with one or more hydroxy, and R¹ is halo;
R² is C₁-C₆ alkyl optionally substituted with one or more NR⁸R⁹, and R¹ is C₁-C₆ alkyl;
R² is C₁-C₆ alkyl optionally substituted with one or more NR⁸R⁹, and R¹ is halo;
R² is C₁-C₆ alkyl optionally substituted with one or more oxo, and R¹ is methyl; or
R² is C₁-C₆ alkyl optionally substituted with one or more C₁-C₆ alkoxy, and R¹ is C₁-C₆ alkyl.

In some embodiments, of the compound of formula AA,

the substituted ring A is

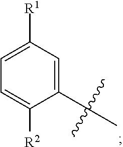

and R¹ and R² are one of the following combinations:
R¹ is 1-hydroxy-2-methylpropan-2-yl, and R² is methyl;
R¹ is 2-hydroxy-2-propyl and R² is methyl;
R¹ is 2-hydroxy-2-propyl and R² is isopropyl;
R¹ is 2-hydroxy-2-propyl and R² is 2-hydroxy-2-propyl;
R¹ is 2-hydroxy-2-propyl and R² is 1-hydroxyethyl;
R¹ is hydroxymethyl and R² is methyl;
R¹ is 1-hydroxyethyl and R² is methyl;
R¹ is 2-hydroxyethyl and R² is methyl;
R¹ is 1-hydroxy-2-propyl and R² is methyl;
R¹ is 2-hydroxy-2-propyl and R² is phenyl;
R¹ is 2-hydroxy-2-propyl and R² is pyridyl;
R¹ is 2-hydroxy-2-propyl and R² is pyrazolyl;
R¹ is 2-hydroxy-2-propyl, and R² is S(O₂)CH₃;
R¹ is 2-hydroxy-2-propyl and R² is chloro;
R¹ is 2-hydroxy-2-propyl and R² is fluoro;
R¹ is 1-hydroxy-1-cyclopropyl, and R² is methyl;
R¹ is 1-hydroxy-1-cyclobutyl, and R² is methyl;
R¹ is 1-hydroxy-1-cyclopentyl, and R² is methyl;
R¹ is 1-hydroxy-1-cyclohexyl, and R² is methyl;
R¹ is morpholinyl, and R² is methyl;
R¹ is 1,3-dioxolan-2-yl, and R² is methyl;
R¹ is 1,3-dioxolan-2-yl, and R² is fluoro;
R¹ is 1,3-dioxolan-2-yl, and R² is chloro;
R¹ is COCH₃, and R² is methyl;
R¹ is 2-methoxy-2-propyl, and R² is methyl;
R¹ is (dimethylamino)methyl, and R² is methyl.
R² is 1-hydroxy-2-methylpropan-2-yl, and R¹ is methyl;
R² is 2-hydroxy-2-propyl and R¹ is methyl;
R² is 2-hydroxy-2-propyl and R¹ is isopropyl;
R² is 2-hydroxy-2-propyl and R¹ is 1-hydroxyethyl;
R² is hydroxymethyl and R¹ is methyl;
R² is 1-hydroxyethyl and R¹ is methyl;
R² is 2-hydroxyethyl and R¹ is methyl;
R² is 1-hydroxy-2-propyl and R¹ is methyl;
R² is 2-hydroxy-2-propyl and R¹ is phenyl;
R² is C₁-C₆ alkyl optionally substituted with one or more hydroxy, and R¹ is 5- to 10-membered heteroaryl;
R² is 2-hydroxy-2-propyl and R¹ is pyridyl;
R² is 2-hydroxy-2-propyl and R¹ is pyrazolyl;
R² is C₁-C₆ alkyl optionally substituted with one or more hydroxy, and R¹ is S(O₂)CH₃;
R² is 2-hydroxy-2-propyl and R¹ is chloro;
R² is 2-hydroxy-2-propyl and R¹ is fluoro;
R² is C₃-C₇ cycloalkyl optionally substituted with one or more hydroxy, and R¹ is C₁-C₆ alkyl;
R² is 1-hydroxy-1-cyclopropyl, and R¹ is methyl;
R² is 1-hydroxy-1-cyclobutyl, and R¹ is methyl;
R² is 1-hydroxy-1-cyclopentyl, and R¹ is methyl;
R² is 1-hydroxy-1-cyclohexyl, and R¹ is methyl;
R² is morpholinyl, and R¹ is methyl;
R² is 1,3-dioxolan-2-yl, and R¹ is methyl;
R² is 1,3-dioxolan-2-yl, and R¹ is fluoro;
R² is 1,3-dioxolan-2-yl, and R¹ is chloro;
R² is C₁-C₆ alkyl optionally substituted with one or more oxo, and R¹ is methyl;
R² is (dimethylamino)methyl, and R¹ is methyl;
R² is COCH₃, and R¹ is methyl; or
R² is 2-methoxy-2-propyl, and R¹ is methyl.

In some embodiments, the optionally substituted ring A is

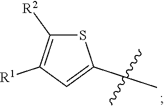

R¹ and R² on adjacent atoms, taken together with the atoms connecting them, independently form one monocyclic or bicyclic C₄-C₁₂ carbocyclic ring or one monocyclic or bicyclic 5- to-12-membered heterocyclic ring that includes from 1-3 heteroatoms and/or heteroatomic groups independently selected from O, NH, NR¹³, S, S(O), and S(O)₂, wherein the carbocyclic ring or heterocyclic ring is optionally independently substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR⁸R⁹, ═NR¹⁰, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR⁸R⁹, wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR⁸R⁹, ═NR¹⁰, COOC₁-C₆ alkyl, C₆-C₁₀ aryl, and CONR⁸R⁹.

In some embodiments, the optionally substituted ring A is

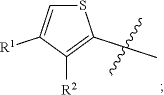

R¹ and R² on adjacent atoms, taken together with the atoms connecting them, independently form one monocyclic or bicyclic C₄-C₁₂ carbocyclic ring or one monocyclic or bicyclic 5- to-12-membered heterocyclic ring that includes from 1-3 heteroatoms and/or heteroatomic groups independently selected from O, NH, NR¹³, S, S(O), and S(O)₂, and wherein the carbocyclic ring or heterocyclic ring is optionally independently substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR⁸R⁹, ═NR¹⁰, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR⁸R⁹, wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR⁸R⁹, ═NR¹⁰, COOC₁-C₆ alkyl, C₆-C₁₀ aryl, and CONR⁸R⁹.

In some embodiments, the optionally substituted ring A is

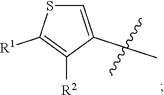

R¹ and R² on adjacent atoms, taken together with the atoms connecting them, independently form one monocyclic or bicyclic C₄-C₁₂ carbocyclic ring or one monocyclic or bicyclic 5- to-12-membered heterocyclic ring that includes from 1-3 heteroatoms and/or heteroatomic groups independently selected from O, NH, NR¹³, S, S(O), and S(O)₂, and wherein the carbocyclic ring or heterocyclic ring is optionally independently substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR⁸R⁹, ═NR¹⁰, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR⁸R⁹, wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR⁸R⁹, ═NR¹⁰, COOC₁-C₆ alkyl, C₆-C₁₀ aryl, and CONR⁸R⁹.

In some embodiments, the optionally substituted ring A is

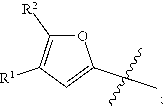

R¹ and R² on adjacent atoms, taken together with the atoms connecting them, independently form one monocyclic or bicyclic C₄-C₁₂ carbocyclic ring or one monocyclic or bicyclic 5- to-12-membered heterocyclic ring that includes from 1-3 heteroatoms and/or heteroatomic groups independently selected from O, NH, NR¹³, S, S(O), and S(O)₂, and wherein the carbocyclic ring or heterocyclic ring is optionally independently substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR⁸R⁹, ═NR¹⁰, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR⁸R⁹, wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR⁸R⁹, ═NR¹⁰, COOC₁-C₆ alkyl, C₆-C₁₀ aryl, and CONR⁸R⁹.

In some embodiments, the optionally substituted ring A is

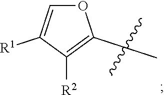

R¹ and R² on adjacent atoms, taken together with the atoms connecting them, independently form one monocyclic or bicyclic C₄-C₁₂ carbocyclic ring or one monocyclic or bicyclic 5- to-12-membered heterocyclic ring that includes from 1-3 heteroatoms and/or heteroatomic groups independently selected from O, NH, NR¹³, S, S(O), and S(O)₂, and wherein the carbocyclic ring or heterocyclic ring is optionally independently substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR⁸R⁹, ═NR¹⁰, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR⁸R⁹, wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR⁸R⁹, ═NR¹⁰, COOC₁-C₆ alkyl, C₆-C₁₀ aryl, and CONR⁸R⁹.

In some embodiments, the optionally substituted ring A is

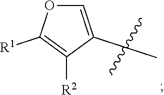

R¹ and R² on adjacent atoms, taken together with the atoms connecting them, independently form one monocyclic or bicyclic C₄-C₁₂ carbocyclic ring or one monocyclic or bicyclic 5- to-12-membered heterocyclic ring that includes from 1-3 heteroatoms and/or heteroatomic groups independently selected from O, NH, NR¹³, S, S(O), and S(O)₂, and wherein the carbocyclic ring or heterocyclic ring is optionally independently substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR⁸R⁹, ═NR¹⁰, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR⁸R⁹, wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR⁸R⁹, ═NR¹⁰, COOC₁-C₆ alkyl, C₆-C₁₀ aryl, and CONR⁸R⁹.

In some embodiments, the optionally substituted ring A is

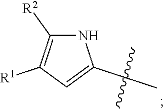

R¹ and R² on adjacent atoms, taken together with the atoms connecting them, independently form one monocyclic or bicyclic C₄-C₁₂ carbocyclic ring or one monocyclic or bicyclic 5- to-12-membered heterocyclic ring that includes from 1-3 heteroatoms and/or heteroatomic groups independently selected from O, NH, NR¹³, S, S(O), and S(O)₂, and wherein the carbocyclic ring or heterocyclic ring is optionally independently substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR⁸R⁹, ═NR¹⁰, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR⁸R⁹, wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR⁸R⁹, ═NR¹⁰, COOC₁-C₆ alkyl, C₆-C₁₀ aryl, and CONR⁸R⁹.

In some embodiments, the optionally substituted ring A is

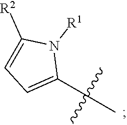

R¹ and R² on adjacent atoms, taken together with the atoms connecting them, independently form one monocyclic or bicyclic C₄-C₁₂ carbocyclic ring or one monocyclic or bicyclic 5- to-12-membered heterocyclic ring that includes from 0-2 heteroatoms and/heteroatomic groups independently selected from O, NH, NR¹³, S, S(O), and S(O)₂ (in addition to the nitrogen atom attached to R¹), and wherein the carbocyclic ring or heterocyclic ring is optionally independently substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR⁸R⁹, ═NR¹⁰, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR⁸R⁹, wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR⁸R⁹, ═NR¹⁰, COOC₁-C₆ alkyl, C₆-C₁₀ aryl, and CONR⁸R⁹.

In some embodiments, the optionally substituted ring A is

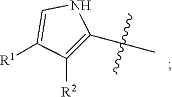

R¹ and R² on adjacent atoms, taken together with the atoms connecting them, independently form one monocyclic or bicyclic C₄-C₁₂ carbocyclic ring or one monocyclic or bicyclic 5- to-12-membered heterocyclic ring that includes from 1-3 heteroatoms and/or heteroatomic groups independently selected from O, NH, NR¹³, S, S(O), and S(O)₂, and wherein the carbocyclic ring or heterocyclic ring is optionally independently substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR⁸R⁹, ═NR¹⁰, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR⁸R⁹, wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR⁸R⁹, ═NR¹⁰, COOC₁-C₆ alkyl, C₆-C₁₀ aryl, and CONR⁸R⁹.

In some embodiments, the optionally substituted ring A is

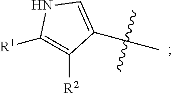

R¹ and R² on adjacent atoms, taken together with the atoms connecting them, independently form one monocyclic or bicyclic C₄-C₁₂ carbocyclic ring or one monocyclic or bicyclic 5- to-12-membered heterocyclic ring that includes from 1-3 heteroatoms and/or heteroatomic groups independently selected from O, NH, NR¹³, S, S(O), and S(O)₂, and wherein the carbocyclic ring or heterocyclic ring is optionally independently substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR⁸R⁹, ═NR¹⁰, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR⁸R⁹, wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR⁸R⁹, ═NR¹⁰, COOC₁-C₆ alkyl, C₆-C₁₀ aryl, and CONR⁸R⁹.

In some embodiments, the optionally substituted ring A is

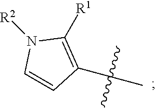

R¹ and R² on adjacent atoms, taken together with the atoms connecting them, independently form one monocyclic or bicyclic C₄-C₁₂ carbocyclic ring or one monocyclic or bicyclic 5- to-12-membered heterocyclic ring that includes from 0-2 heteroatoms and/heteroatomic groups independently selected from O, NH, NR¹³, S, S(O), and S(O)₂ (in addition to the nitrogen atom attached to R²), and wherein the carbocyclic ring or heterocyclic ring is optionally independently substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR⁸R⁹, ═NR¹⁰, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR⁸R⁹, wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR⁸R⁹, ═NR¹⁰, COOC₁-C₆ alkyl, C₆-C₁₀ aryl, and CONR⁸R⁹.

In some embodiments, the optionally substituted ring A is

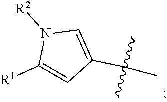

R¹ and R² on adjacent atoms, taken together with the atoms connecting them, independently form one monocyclic or bicyclic C₄-C₁₂ carbocyclic ring or one monocyclic or bicyclic 5- to-12-membered heterocyclic ring that includes from 0-2 heteroatoms and/heteroatomic groups independently selected from O, NH, NR¹³, S, S(O), and S(O)₂ (in addition to the nitrogen attached to R²), and wherein the carbocyclic ring or heterocyclic ring is optionally independently substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR⁸R⁹, ═NR¹⁰, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR⁸R⁹, wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR⁸R⁹, ═NR¹⁰, COOC₁-C₆ alkyl, C₆-C₁₀ aryl, and CONR⁸R⁹.

In some embodiments, the optionally substituted ring A is

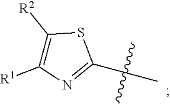

R¹ and R² on adjacent atoms, taken together with the atoms connecting them, independently form one monocyclic or bicyclic C₄-C₁₂ carbocyclic ring or one monocyclic or bicyclic 5- to-12-membered heterocyclic ring that includes from 1-3 heteroatoms and/or heteroatomic groups independently selected from O, NH, NR¹³, S, S(O), and S(O)₂, and wherein the carbocyclic ring or heterocyclic ring is optionally independently substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR⁸R⁹, ═NR¹⁰, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR⁸R⁹, wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR⁸R⁹, ═NR¹⁰, COOC₁-C₆ alkyl, C₆-C₁₀ aryl, and CONR⁸R⁹.

In some embodiments, the optionally substituted ring A is

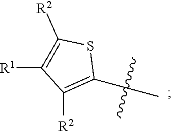

one pair of R¹ and R² on adjacent atoms, taken together with the atoms connecting them, independently form one monocyclic or bicyclic C₄-C₁₂ carbocyclic ring or one monocyclic or bicyclic 5- to-12-membered heterocyclic ring that includes from 1-3 heteroatoms and/or heteroatomic groups independently selected from O, NH, NR¹³, S, S(O), and S(O)₂, and wherein the carbocyclic ring or heterocyclic ring is optionally independently substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR⁸R⁹, ═NR¹⁰, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR⁸R⁹, wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR⁸R⁹, ═NR¹⁰, COOC₁-C₆ alkyl, C₆-C₁₀ aryl, and CONR⁸R⁹.

In some embodiments, the optionally substituted ring A is

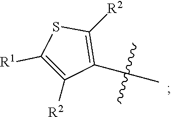

R¹ and R² on adjacent atoms, taken together with the atoms connecting them, independently form one monocyclic or bicyclic C₄-C₁₂ carbocyclic ring or one monocyclic or bicyclic 5- to-12-membered heterocyclic ring that includes from 1-3 heteroatoms and/or heteroatomic groups independently selected from O, NH, NR¹³, S, S(O), and S(O)₂, and wherein the carbocyclic ring or heterocyclic ring is optionally independently substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR⁸R⁹, ═NR¹⁰, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR⁸R⁹, wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR⁸R⁹, ═NR¹⁰, COOC₁-C₆ alkyl, C₆-C₁₀ aryl, and CONR⁸R⁹.

In some embodiments, the optionally substituted ring A is

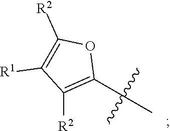

one pair of R¹ and R² on adjacent atoms, taken together with the atoms connecting them, independently form one monocyclic or bicyclic C₄-C₁₂ carbocyclic ring or one monocyclic or bicyclic 5- to-12-membered heterocyclic ring that includes from 1-3 heteroatoms and/or heteroatomic groups independently selected from O, NH, NR¹³, S, S(O), and S(O)₂, and wherein the carbocyclic ring or heterocyclic ring is optionally independently substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR⁸R⁹, ═NR¹⁰, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR⁸R⁹, wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR⁸R⁹, ═NR¹⁰, COOC₁-C₆ alkyl, C₆-C₁₀ aryl, and CONR⁸R⁹.

In some embodiments, the optionally substituted ring A is

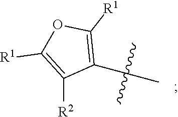

R¹ and R² on adjacent atoms, taken together with the atoms connecting them, independently form one monocyclic or bicyclic C₄-C₁₂ carbocyclic ring or one monocyclic or bicyclic 5- to-12-membered heterocyclic ring that includes from 1-3 heteroatoms and/or heteroatomic groups independently selected from O, NH, NR¹³, S, S(O), and S(O)₂, wherein the carbocyclic ring or heterocyclic ring is optionally independently substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR⁸R⁹, ═NR¹⁰, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR⁸R⁹, wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR⁸R⁹, ═NR¹⁰, COOC₁-C₆ alkyl, C₆-C₁₀ aryl, and CONR⁸R⁹.

In some embodiments, the optionally substituted ring A is

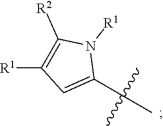

one pair of R¹ and R² on adjacent atoms, taken together with the atoms connecting them, independently form one monocyclic or bicyclic C₄-C₁₂ carbocyclic ring or one monocyclic or bicyclic 5- to-12-membered heterocyclic ring wherein a) when each of the adjacent atoms is a carbon atom, then the heterocyclic ring includes from 1-3 heteroatoms and/or heteroatomic groups independently selected from O, NH, NR¹³, S, S(O), and S(O)₂; and b) when one of the adjacent atoms is a nitrogen atom, then the heterocyclic ring includes from 0-2 heteroatoms and/or heteroatomic groups independently selected from O, NH, NR¹³, S, S(O), and S(O)₂ (in addition to the aforementioned nitrogen atom attached to R¹), and wherein the carbocyclic ring or heterocyclic ring is optionally independently substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR⁸R⁹, —NR¹⁰, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR⁸R⁹, wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR⁸R⁹, ═NR¹⁰, COOC₁-C₆ alkyl, C₆-C₁₀ aryl, and CONR⁸R⁹.

In some embodiments, the optionally substituted ring A is

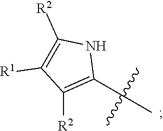

one pair of R¹ and R² on adjacent atoms, taken together with the atoms connecting them, independently form one monocyclic or bicyclic C₄-C₁₂ carbocyclic ring or one monocyclic or bicyclic 5- to-12-membered heterocyclic ring that includes from 1-3 heteroatoms and/or heteroatomic groups independently selected from O, NH, NR¹³, S, S(O), and S(O)₂, and wherein the carbocyclic ring or heterocyclic ring is optionally independently substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR⁸R⁹, ═NR¹⁰, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR⁸R⁹, wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR⁸R⁹, ═NR¹⁰, COOC₁-C₆ alkyl, C₆-C₁₀ aryl, and CONR⁸R⁹.

In some embodiments, the optionally substituted ring A is

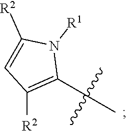

R¹ and R² on adjacent atoms, taken together with the atoms connecting them, independently form one monocyclic or bicyclic C₄-C₁₂ carbocyclic ring or one monocyclic or bicyclic 5- to-12-membered heterocyclic ring that includes from 0-2 heteroatoms and/heteroatomic groups independently selected from O, NH, NR¹³, S, S(O), and S(O)₂ (in addition to the nitrogen atom attached to R¹), and wherein the carbocyclic ring or heterocyclic ring is optionally independently substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR⁸R⁹, ═NR¹⁰, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR⁸R⁹, wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR⁸R⁹, ═NR¹⁰, COOC₁-C₆ alkyl, C₆-C₁₀ aryl, and CONR⁸R⁹.

In some embodiments, the optionally substituted ring A is

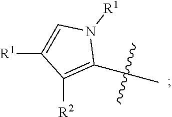

R¹ and R² on adjacent atoms, taken together with the atoms connecting them, independently form one monocyclic or bicyclic C₄-C₁₂ carbocyclic ring or one monocyclic or bicyclic 5- to-12-membered heterocyclic ring that includes from 1-3 heteroatoms and/heteroatomic groups independently selected from O, NH, NR¹³, S, S(O), and S(O)₂, and wherein the carbocyclic ring or heterocyclic ring is optionally independently substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR⁸R⁹, ═NR¹⁰, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR⁸R⁹, wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR⁸R⁹, ═NR¹⁰, COOC₁-C₆ alkyl, C₆-C₁₀ aryl, and CONR⁸R⁹.

In some embodiments, the optionally substituted ring A is

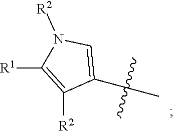

one pair of R¹ and R² on adjacent atoms, taken together with the atoms connecting them, independently form one monocyclic or bicyclic C₄-C₁₂ carbocyclic ring or one monocyclic or bicyclic 5- to-12-membered heterocyclic ring wherein a) when each of the adjacent atoms is a carbon atom, then the heterocyclic ring includes from 1-3 heteroatoms and/or heteroatomic groups independently selected from O, NH, NR¹³, S, S(O), and S(O)₂; and b) when one of the adjacent atoms is a nitrogen atom, then the heterocyclic ring includes from 0-2 heteroatoms and/or heteroatomic groups independently selected from O, NH, NR¹³, S, S(O), and S(O)₂ (in addition to the aforementioned nitrogen atom attached to R²), and wherein the carbocyclic ring or heterocyclic ring is optionally independently substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR⁸R⁹, ═NR¹⁰, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR⁸R⁹, wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR⁸R⁹, ═NR¹⁰, COOC₁-C₆ alkyl, C₆-C₁₀ aryl, and CONR⁸R⁹.

In some embodiments, the optionally substituted ring A is

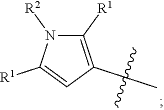

one pair of R¹ and R² on adjacent atoms, taken together with the atoms connecting them, independently form one monocyclic or bicyclic C₄-C₁₂ carbocyclic ring or one monocyclic or bicyclic 5- to-12-membered heterocyclic ring that includes from 0-2 heteroatoms and/heteroatomic groups independently selected from O, NH, NR¹³, S, S(O), and S(O)₂ (in addition to the nitrogen atom(s) attached to R²), and wherein the carbocyclic ring or heterocyclic ring is optionally independently substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR⁸R⁹, ═NR¹⁰, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR⁸R⁹, wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR⁸R⁹, ═NR¹⁰, COOC₁-C₆ alkyl, C₆-C₁₀ aryl, and CONR⁸R⁹.

In some embodiments of the compound of formula AA,

the substituted ring B is

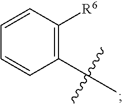

and R⁶ is selected from:
C₁-C₆ alkyl, C₁-C₆ alkyl substituted with one or more halo, C₁-C₆ alkoxy, C₁-C₆ alkoxy substituted with one or more halo, C₃-C₇ cycloalkyl, halo, and cyano.

In some embodiments of the compound of formula AA,

the substituted ring B is

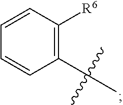

and R⁶ is selected from:
isopropyl, ethyl, methyl, trifluoromethyl, trifluoromethoxy, cyclopropyl, halo, chloro, and fluoro.

In some embodiments of the compound of formula AA,

the substituted ring B is

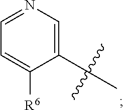

and R⁶ is selected from:
C₁-C₆ alkyl, C₁-C₆ alkyl substituted with one or more halo, C₁-C₆ alkoxy, C₁-C₆ alkoxy substituted with one or more halo, C₃-C₇ cycloalkyl, halo, and cyano.

In some embodiments of the compound of formula AA,

the substituted ring B is

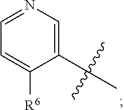

and R⁶ is selected from:
isopropyl, ethyl, methyl, trifluoromethyl, trifluoromethoxy, cyclopropyl, halo, chloro, and fluoro.

In some embodiments of the compound of formula AA,

the substituted ring B is

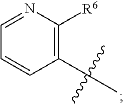

and R⁶ is selected from:
C₁-C₆ alkyl, C₁-C₆ alkyl substituted with one or more halo, C₁-C₆ alkoxy, C₁-C₆ alkoxy substituted with one or more halo, C₃-C₇ cycloalkyl, halo, and cyano.

In some embodiments of the compound of formula AA,

the substituted ring B is

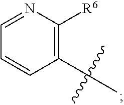

and R⁶ is selected from:
isopropyl, ethyl, methyl, trifluoromethyl, trifluoromethoxy, cyclopropyl, halo, chloro, and fluoro.

In some embodiments of the compound of formula AA,

the substituted ring B is

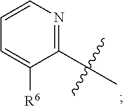

and R⁶ is selected from:
C₁-C₆ alkyl, C₁-C₆ alkyl substituted with one or more halo, C₁-C₆ alkoxy, C₁-C₆ alkoxy substituted with one or more halo, C₃-C₇ cycloalkyl, halo, and cyano.

In some embodiments of the compound of formula AA,

the substituted ring B is

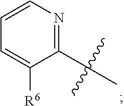

and R⁶ is selected from:
isopropyl, ethyl, methyl, trifluoromethyl, trifluoromethoxy, cyclopropyl, halo, chloro, and fluoro.

In some embodiments of the compound of formula AA,

the substituted ring B is

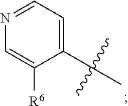

and R⁶ is selected from:
C₁-C₆ alkyl, C₁-C₆ alkyl substituted with one or more halo, C₁-C₆ alkoxy, C₁-C₆ alkoxy substituted with one or more halo, C₃-C₇ cycloalkyl, halo, and cyano.

In some embodiments of the compound of formula AA,

the substituted ring B is

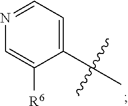

and R⁶ is selected from:
isopropyl, ethyl, methyl, trifluoromethyl, trifluoromethoxy, cyclopropyl, halo, chloro, and fluoro.

In some embodiments, the substituted ring B is

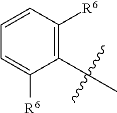

and each R⁶ is independently selected from the group consisting of: C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO-C₁-C₆ alkyl; CONR⁸R⁹, and 4- to 6-membered heterocycloalkyl, wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl.

In some embodiments, the substituted ring B is

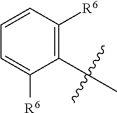

and each R⁶ is independently selected from the group consisting of: C₁-C₆ alkyl, C₃-C₇ cycloalkyl, C₁-C₆ haloalkyl, C₁-C₆ alkoxy, C₁-C₆ haloalkoxy, wherein the C₁-C₆ alkyl, C₁-C₆ haloalkyl, and C₃-C₇ cycloalkyl is optionally substituted with one or more substituents each independently selected from hydroxy, halo, CN, or oxo.

In some embodiments, the substituted ring B is

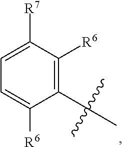

wherein each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO-C₁-C₆ alkyl; CONR⁸R⁹, and 4- to 6-membered heterocycloalkyl,

wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;
wherein R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy;

or R⁶ and R⁷, taken together with the atoms connecting them, independently form C₄-C₇ carbocyclic ring or at least one 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, the substituted ring B is

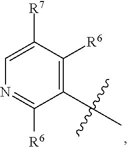

wherein each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO-C₁-C₆ alkyl; CONR⁸R⁹, and 4- to 6-membered heterocycloalkyl,

wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;
wherein R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy;

or R⁶ and R⁷, taken together with the atoms connecting them, independently form C₄-C₇ carbocyclic ring or at least one 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, the substituted ring B

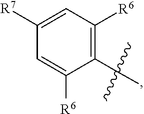

is wherein each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO-C₁-C₆ alkyl; CONR⁸R⁹, and 4- to 6-membered heterocycloalkyl,

wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;
wherein R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy.

In some embodiments, the substituted ring B is

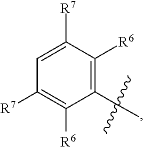

wherein each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO-C₁-C₆ alkyl; CONR⁸R⁹, and 4- to 6-membered heterocycloalkyl,

wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;
wherein each R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy;

or at least one pair of R⁶ and R⁷ on adjacent atoms, taken together with the atoms connecting them, independently form at least one C₄-C₇ carbocyclic ring or at least one 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, ═NR¹⁰, COOC₁-C₆ alkyl, C₆-C₁₀ aryl, and CONR⁸R⁹.

In some embodiments, the substituted ring B is

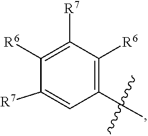

wherein each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO-C₁-C₆ alkyl; CONR⁸R⁹, and 4- to 6-membered heterocycloalkyl,

wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;
wherein each R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy;

or at least one pair of R⁶ and R⁷ on adjacent atoms, taken together with the atoms connecting them, independently form at least one C₄-C₇ carbocyclic ring or at least one 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, ═NR¹⁰, COOC₁-C₆ alkyl, C₆-C₁₀ aryl, and CONR⁸R⁹.

In some embodiments, the substituted ring B is

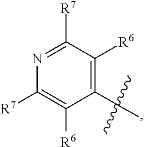

wherein each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO-C₁-C₆ alkyl; CONR⁸R⁹, and 4- to 6-membered heterocycloalkyl,

wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;
wherein each R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy;

or at least one pair of R⁶ and R⁷ on adjacent atoms, taken together with the atoms connecting them, independently form at least one C₄-C₇ carbocyclic ring or at least one 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, the substituted ring B is

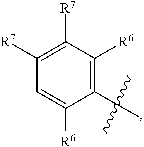

wherein each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO-C₁-C₆ alkyl; CONR⁸R⁹, and 4- to 6-membered heterocycloalkyl,

wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;
wherein each R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy;
or R⁶ and R⁷, taken together with the atoms connecting them, independently form a C₄-C₇ carbocyclic ring or at least one 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In some embodiments, the substituted ring B is

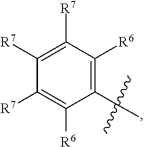

wherein each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO-C₁-C₆ alkyl; CONR⁸R⁹, and 4- to 6-membered heterocycloalkyl,

wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;
wherein each R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy;
or at least one pair of R⁶ and R⁷ on adjacent atoms, taken together with the atoms connecting them, independently form at least one C₄-C₇ carbocyclic ring or at least one 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹.

In some embodiments, of the compound of formula AA,

the substituted ring B is

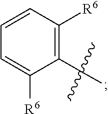

and the two R⁶ are one of the following combinations:
One R⁶ is C₁-C₆ alkyl, and the other R⁶ is C₁-C₆ alkyl optionally substituted with one or more halo;
One R⁶ is C₁-C₆ alkyl and the other R⁶ is C₁-C₆ alkyl;
One R⁶ is C₁-C₆ alkyl, and the other R⁶ is C₁-C₆ alkyl substituted with one or more halo;
One R⁶ is C₁-C₆ alkyl, and the other R⁶ is C₃-C₇ cycloalkyl;
One R⁶ is C₁-C₆ alkyl, and the other R⁶ is halo;
One R⁶ is C₁-C₆ alkyl, and the other R⁶ is cyano;
One R⁶ is C₃-C₇ cycloalkyl, and the other R⁶ is C₃-C₇ cycloalkyl;
One R⁶ is C₃-C₇ cycloalkyl, and the other R⁶ is halo;
One R⁶ is cyclopropyl and the other R⁶ is halo;
One R⁶ is C₁-C₆ alkyl, and the other R⁶ is C₁-C₆ alkoxy optionally substituted with one or more halo;
One R⁶ is C₁-C₆ alkyl, and the other R⁶ is C₁-C₆ alkoxy;
One R⁶ is C₁-C₆ alkyl, and the other R⁶ is C₁-C₆ alkoxy substituted with one or more halo;
One R⁶ is halo, and the other R⁶ is C₁-C₆ haloalkyl;
One R⁶ is halo, and the other R⁶ is C₁-C₆ haloalkoxy;
One R⁶ is C₁-C₆ alkoxy; and the other R⁶ is halo;
One R⁶ is C₁-C₆ alkoxy; and the other R⁶ is chloro.

In some embodiments, of the compound of formula AA,

the substituted ring B is

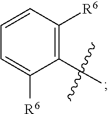

and the two R⁶ are one of the following combinations:
One R⁶ is isopropyl; and the other R⁶ is methyl;
One R⁶ is isopropyl; and the other R⁶ is n-propyl;
One R⁶ is isopropyl; and the other R⁶ is isopropyl;
One R⁶ is isopropyl; and the other R⁶ is trifluoromethyl;
One R⁶ is isopropyl; and the other R⁶ is cyclopropyl;
One R⁶ is isopropyl; and the other R⁶ is chloro;
One R⁶ is isopropyl; and the other R⁶ is fluoro;
One R⁶ is ethyl; and the other R⁶ is fluoro;
One R⁶ is isopropyl; and the other R⁶ is cyano;
One R⁶ is cyclopropyl; and the other R⁶ is cyclopropyl;
One R⁶ is cyclopropyl; and the other R⁶ is chloro;
One R⁶ is cyclopropyl; and the other R⁶ is fluoro;
One R⁶ is isopropyl; and the other R⁶ is methoxy;
One R⁶ is isopropyl; and the other R⁶ is methoxy; or
One R⁶ is isopropyl; and the other R⁶ is trifluoromethoxy.

In some embodiments, of the compound of formula AA,

the substituted ring B is

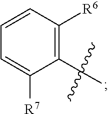

and R⁶ and R⁷ are one of the following combinations:
R⁶ is C₁-C₆ alkyl, and R⁷ is C₁-C₆ alkyl optionally substituted with one or more halo;
R⁶ is C₁-C₆ alkyl and R⁷ is C₁-C₆ alkyl;
R⁶ is C₁-C₆ alkyl, and R⁷ is C₁-C₆ alkyl substituted with one or more halo;
R⁶ is C₁-C₆ alkyl, and R⁷ is C₃-C₇ cycloalkyl;
R⁶ is C₁-C₆ alkyl, and R⁷ is halo;
R⁶ is C₁-C₆ alkyl, and R⁷ is cyano;
R⁶ is C₃-C₇ cycloalkyl, and R⁷ is C₃-C₇ cycloalkyl;
R⁶ is C₃-C₇ cycloalkyl, and R⁷ is halo;
R⁶ is cyclopropyl and R⁷ is halo;
R⁶ is C₁-C₆ alkyl, and R⁷ is C₁-C₆ alkoxy optionally substituted with one or more halo;
R⁶ is C₁-C₆ alkyl, and R⁷ is C₁-C₆ alkoxy;
R⁶ is C₁-C₆ alkyl, and R⁷ is C₁-C₆ alkoxy substituted with one or more halo;
R⁶ is halo, and R⁷ is C₁-C₆ haloalkyl;
R⁶ is halo, and R⁷ is C₁-C₆ haloalkoxy;
R⁶ is C₁-C₆ alkoxy; and R⁷ is halo;
R⁶ is C₁-C₆ alkoxy; and R⁷ is chloro;
R⁷ is C₁-C₆ alkyl, and R⁶ is C₁-C₆ alkyl optionally substituted with one or more halo;
R⁷ is C₁-C₆ alkyl, and R⁶ is C₁-C₆ alkyl substituted with one or more halo;
R⁷ is C₁-C₆ alkyl, and R⁶ is C₃-C₇ cycloalkyl;
R⁷ is C₁-C₆ alkyl, and R⁶ is halo;
R⁷ is C₁-C₆ alkyl and R⁶ is halo;
R⁷ is C₁-C₆ alkyl, and R⁶ is cyano;
R⁷ is C₃-C₇ cycloalkyl, and R⁶ is C₃-C₇ cycloalkyl;
R⁷ is C₃-C₇ cycloalkyl, and R⁶ is halo;
R⁷ is C₃-C₇ cycloalkyl and R⁶ is halo;
R⁷ is C₁-C₆ alkyl, and R⁶ is C₁-C₆ alkoxy optionally substituted with one or more halo;
R⁷ is C₁-C₆ alkyl, and R⁶ is C₁-C₆ alkoxy;
R⁷ is C₁-C₆ alkyl, and R⁶ is C₁-C₆ alkoxy substituted with one or more halo;
R⁷ is halo, and R⁶ is C₁-C₆ haloalkyl;
R⁷ is halo, and R⁶ is C₁-C₆ haloalkoxy;
R⁷ is C₁-C₆ alkoxy; and R⁶ is halo; or
R⁷ is C₁-C₆ alkoxy; and R⁶ is chloro.

In some embodiments, of the compound of formula AA,

the substituted ring B is

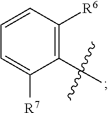

and R⁶ and R⁷ are one of the following combinations:
R⁶ is isopropyl; and R⁷ is methyl;
R⁶ is isopropyl; and R⁷ is isopropyl;
R⁶ is isopropyl; and R⁷ is trifluoromethyl;
R⁶ is isopropyl; and R⁷ is cyclopropyl;
R⁶ is isopropyl; and R⁷ is chloro;
R⁶ is isopropyl; and R⁷ is fluoro;
R⁶ is ethyl; and R⁷ is fluoro;
R⁶ is isopropyl; and R⁷ is cyano;
R⁶ is cyclopropyl; and R⁷ is cyclopropyl;
R⁶ is cyclopropyl; and R⁷ is chloro;
R⁶ is cyclopropyl; and R⁷ is fluoro; R⁶ is isopropyl; and R⁷ is methoxy;
R⁶ is isopropyl; and R⁷ is trifluoromethoxy;
R⁶ is chloro; and R⁷ is trifluoromethyl;
R⁶ is chloro; and R⁷ is trifluoromethoxy;
R⁷ is isopropyl; and R⁶ is methyl;
R⁷ is isopropyl; and R⁶ is trifluoromethyl;
R⁷ is isopropyl; and R⁶ is cyclopropyl;
R⁷ is isopropyl; and R⁶ is chloro;
R⁷ is ethyl; and R⁶ is fluoro;
R⁷ is isopropyl; and R⁶ is cyano;
R⁷ is cyclopropyl; and R⁶ is cyclopropyl;
R⁷ is cyclopropyl; and R⁶ is chloro;
R⁷ is cyclopropyl; and R⁶ is fluoro;
R⁷ is isopropyl; and R⁶ is methoxy;
R⁷ is isopropyl; and R⁶ is trifluoromethoxy;
R⁷ is chloro; and R⁶ is trifluoromethyl; or
R⁷ is chloro; and R⁶ is trifluoromethoxy.

In some embodiments, of the compound of formula AA,

the substituted ring B is

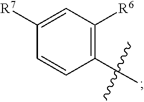

and R⁶ and R⁷ are one of the following combinations:
R⁶ is C₁-C₆ alkyl, and R⁷ is C₁-C₆ alkyl optionally substituted with one or more halo;
R⁶ is C₁-C₆ alkyl and R⁷ is C₁-C₆ alkyl;
R⁶ is C₁-C₆ alkyl, and R⁷ is C₁-C₆ alkyl substituted with one or more halo;
R⁶ is C₁-C₆ alkyl, and R⁷ is C₃-C₇ cycloalkyl;
R⁶ is C₁-C₆ alkyl, and R⁷ is halo;
R⁶ is C₁-C₆ alkyl, and R⁷ is cyano;
R⁶ is C₃-C₇ cycloalkyl, and R⁷ is C₃-C₇ cycloalkyl;
R⁶ is C₃-C₇ cycloalkyl, and R⁷ is halo;
R⁶ is cyclopropyl and R⁷ is halo;
R⁶ is C₁-C₆ alkyl, and R⁷ is C₁-C₆ alkoxy optionally substituted with one or more halo;
R⁶ is C₁-C₆ alkyl, and R⁷ is C₁-C₆ alkoxy;
R⁶ is C₁-C₆ alkyl, and R⁷ is C₁-C₆ alkoxy substituted with one or more halo;
R⁶ is halo, and R⁷ is C₁-C₆ haloalkyl;
R⁶ is halo, and R⁷ is C₁-C₆ haloalkoxy;
R⁶ is C₁-C₆ alkoxy; and R⁷ is halo;
R⁶ is C₁-C₆ alkoxy; and R⁷ is chloro;
R⁷ is C₁-C₆ alkyl, and R⁶ is C₁-C₆ alkyl optionally substituted with one or more halo;
R⁷ is C₁-C₆ alkyl, and R⁶ is C₁-C₆ alkyl substituted with one or more halo;
R⁷ is C₁-C₆ alkyl, and R⁶ is C₃-C₇ cycloalkyl;
R⁷ is C₁-C₆ alkyl, and R⁶ is halo;
R⁷ is C₁-C₆ alkyl and R⁶ is halo;
R⁷ is C₁-C₆ alkyl, and R⁶ is cyano;
R⁷ is C₃-C₇ cycloalkyl, and R⁶ is C₃-C₇ cycloalkyl;
R⁷ is C₃-C₇ cycloalkyl, and R⁶ is halo;
R⁷ is C₃-C₇ cycloalkyl and R⁶ is halo;
R⁷ is C₁-C₆ alkyl, and R⁶ is C₁-C₆ alkoxy optionally substituted with one or more halo;
R⁷ is C₁-C₆ alkyl, and R⁶ is C₁-C₆ alkoxy;
R⁷ is C₁-C₆ alkyl, and R⁶ is C₁-C₆ alkoxy substituted with one or more halo;
R⁷ is halo, and R⁶ is C₁-C₆ haloalkyl;
R⁷ is halo, and R⁶ is C₁-C₆ haloalkoxy;
R⁷ is C₁-C₆ alkoxy; and R⁶ is halo; or
R⁷ is C₁-C₆ alkoxy; and R⁶ is chloro.

In some embodiments, of the compound of formula AA,

the substituted ring B is

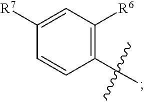

and R⁶ and R⁷ are one of the following combinations:
R⁶ is isopropyl; and R⁷ is methyl;
R⁶ is isopropyl; and R⁷ is isopropyl;
R⁶ is isopropyl; and R⁷ is trifluoromethyl;
R⁶ is isopropyl; and R⁷ is cyclopropyl;
R⁶ is isopropyl; and R⁷ is chloro;
R⁶ is isopropyl; and R⁷ is fluoro;
R⁶ is ethyl; and R⁷ is fluoro;
R⁶ is isopropyl; and R⁷ is cyano;
R⁶ is cyclopropyl; and R⁷ is cyclopropyl;
R⁶ is cyclopropyl; and R⁷ is chloro;
R⁶ is cyclopropyl; and R⁷ is fluoro;
R⁶ is isopropyl; and R⁷ is methoxy;
R⁶ is isopropyl; and R⁷ is trifluoromethoxy;
R⁶ is chloro; and R⁷ is trifluoromethyl;
R⁶ is chloro; and R⁷ is trifluoromethoxy;
R⁷ is isopropyl; and R⁶ is methyl;
R⁷ is isopropyl; and R⁶ is trifluoromethyl;
R⁷ is isopropyl; and R⁶ is cyclopropyl;
R⁷ is isopropyl; and R⁶ is chloro;
R⁷ is ethyl; and R⁶ is fluoro;
R⁷ is isopropyl; and R⁶ is cyano;
R⁷ is cyclopropyl; and R⁶ is cyclopropyl;
R⁷ is cyclopropyl; and R⁶ is chloro;
R⁷ is cyclopropyl; and R⁶ is fluoro;
R⁷ is isopropyl; and R⁶ is methoxy;
R⁷ is isopropyl; and R⁶ is trifluoromethoxy;
R⁷ is chloro; and R⁶ is trifluoromethyl; or
R⁷ is chloro; and R⁶ is trifluoromethoxy.

In some embodiments, of the compound of formula AA,

the substituted ring B is

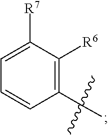

and R⁶ and R⁷ are one of the following combinations:
R⁶ is C₁-C₆ alkyl, and R⁷ is C₁-C₆ alkyl optionally substituted with one or more halo;
R⁶ is C₁-C₆ alkyl and R⁷ is C₁-C₆ alkyl;
R⁶ is C₁-C₆ alkyl, and R⁷ is C₁-C₆ alkyl substituted with one or more halo;
R⁶ is C₁-C₆ alkyl, and R⁷ is C₃-C₇ cycloalkyl;
R⁶ is C₁-C₆ alkyl, and R⁷ is halo;
R⁶ is C₁-C₆ alkyl, and R⁷ is cyano;
R⁶ is C₃-C₇ cycloalkyl, and R⁷ is C₃-C₇ cycloalkyl;
R⁶ is C₃-C₇ cycloalkyl, and R⁷ is halo;
R⁶ is cyclopropyl and R⁷ is halo;
R⁶ is C₁-C₆ alkyl, and R⁷ is C₁-C₆ alkoxy optionally substituted with one or more halo;
R⁶ is C₁-C₆ alkyl, and R⁷ is C₁-C₆ alkoxy;
R⁶ is C₁-C₆ alkyl, and R⁷ is C₁-C₆ alkoxy substituted with one or more halo;
R⁶ is halo, and R⁷ is C₁-C₆ haloalkyl;
R⁶ is halo, and R⁷ is C₁-C₆ haloalkoxy;
R⁶ is C₁-C₆ alkoxy; and R⁷ is halo;
R⁶ is C₁-C₆ alkoxy; and R⁷ is chloro;
R⁷ is C₁-C₆ alkyl, and R⁶ is C₁-C₆ alkyl optionally substituted with one or more halo;
R⁷ is C₁-C₆ alkyl, and R⁶ is C₁-C₆ alkyl substituted with one or more halo;
R⁷ is C₁-C₆ alkyl, and R⁶ is C₃-C₇ cycloalkyl;
R⁷ is C₁-C₆ alkyl, and R⁶ is halo;
R⁷ is C₁-C₆ alkyl and R⁶ is halo;
R⁷ is C₁-C₆ alkyl, and R⁶ is cyano;
R⁷ is C₃-C₇ cycloalkyl, and R⁶ is C₃-C₇ cycloalkyl;
R⁷ is C₃-C₇ cycloalkyl, and R⁶ is halo;
R⁷ is C₃-C₇ cycloalkyl and R⁶ is halo;
R⁷ is C₁-C₆ alkyl, and R⁶ is C₁-C₆ alkoxy optionally substituted with one or more halo;
R⁷ is C₁-C₆ alkyl, and R⁶ is C₁-C₆ alkoxy;
R⁷ is C₁-C₆ alkyl, and R⁶ is C₁-C₆ alkoxy substituted with one or more halo;
R⁷ is halo, and R⁶ is C₁-C₆ haloalkyl;
R⁷ is halo, and R⁶ is C₁-C₆ haloalkoxy;
R⁷ is C₁-C₆ alkoxy; and R⁶ is halo; or
R⁷ is C₁-C₆ alkoxy; and R⁶ is chloro;
R⁶ and R⁷ on adjacent atoms taken together with the atoms connecting them form a C₄-C₆ aliphatic carbocyclic ring optionally substituted with one or more hydroxy, oxo, or C₁-C₆ alkyl; or
R⁶ and R⁷ on adjacent atoms taken together with the atoms connecting them form a 5-to-6-membered heterocyclic ring containing 1 heteroatom independently selected from O, N, and S, wherein the heterocyclic ring optionally substituted with one or more hydroxy, oxo, or C₁-C₆ alkyl.

In some embodiments, of the compound of formula AA,

the substituted ring B is

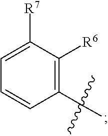

and R⁶ and R⁷ are one of the following combinations:
R⁶ is isopropyl; and R⁷ is methyl;
R⁶ is isopropyl; and R⁷ is isopropyl;
R⁶ is isopropyl; and R⁷ is trifluoromethyl;
R⁶ is isopropyl; and R⁷ is cyclopropyl;
R⁶ is isopropyl; and R⁷ is chloro;
R⁶ is isopropyl; and R⁷ is fluoro;
R⁶ is ethyl; and R⁷ is fluoro;
R⁶ is isopropyl; and R⁷ is cyano;
R⁶ is cyclopropyl; and R⁷ is cyclopropyl;
R⁶ is cyclopropyl; and R⁷ is chloro;
R⁶ is cyclopropyl; and R⁷ is fluoro;
R⁶ is isopropyl; and R⁷ is methoxy;
R⁶ is isopropyl; and R⁷ is trifluoromethoxy;
R⁶ is chloro; and R⁷ is trifluoromethyl;
R⁶ is chloro; and R⁷ is trifluoromethoxy;
R⁷ is isopropyl; and R⁶ is methyl;
R⁷ is isopropyl; and R⁶ is trifluoromethyl;
R⁷ is isopropyl; and R⁶ is cyclopropyl;
R⁷ is isopropyl; and R⁶ is chloro;
R⁷ is ethyl; and R⁶ is fluoro;
R⁷ is isopropyl; and R⁶ is cyano;
R⁷ is cyclopropyl; and R⁶ is cyclopropyl;
R⁷ is cyclopropyl; and R⁶ is chloro;
R⁷ is cyclopropyl; and R⁶ is fluoro;
R⁷ is isopropyl; and R⁶ is methoxy;
R⁷ is isopropyl; and R⁶ is trifluoromethoxy;
R⁷ is chloro; and R⁶ is trifluoromethyl;
R⁷ is chloro; and R⁶ is trifluoromethoxy;
R⁶ and R⁷ on adjacent atoms taken together with the atoms connecting them form a C₄ aliphatic carbocyclic ring;
R⁶ and R⁷ on adjacent atoms taken together with the atoms connecting them form a C₅ aliphatic carbocyclic ring;
R⁶ and R⁷ on adjacent atoms taken together with the atoms connecting them form a C₆ aliphatic carbocyclic ring;
R⁶ and R⁷ on adjacent atoms taken together with the atoms connecting them form a 5-membered heterocyclic ring containing 1 heteroatoms independently selected from O, N, and S;
R⁶ and R⁷ on adjacent atoms taken together with the atoms connecting them form a 6-membered heterocyclic ring containing 1 heteroatoms independently selected from O, N, and S; or
R⁶ and R⁷ on adjacent atoms taken together with the atoms connecting them form a C₅ aliphatic carbocyclic ring.

In some embodiments, of the compound of formula AA,

the substituted ring B is

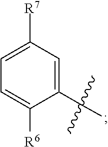

and R⁶ and R⁷ are one of the following combinations:
R⁶ is C₁-C₆ alkyl, and R⁷ is C₁-C₆ alkyl optionally substituted with one or more halo;
R⁶ is C₁-C₆ alkyl and R⁷ is C₁-C₆ alkyl;
R⁶ is C₁-C₆ alkyl, and R⁷ is C₁-C₆ alkyl substituted with one or more halo;
R⁶ is C₁-C₆ alkyl, and R⁷ is C₃-C₇ cycloalkyl;
R⁶ is C₁-C₆ alkyl, and R⁷ is halo;
R⁶ is C₁-C₆ alkyl, and R⁷ is cyano;
R⁶ is C₃-C₇ cycloalkyl, and R⁷ is C₃-C₇ cycloalkyl;
R⁶ is C₃-C₇ cycloalkyl, and R⁷ is halo;
R⁶ is cyclopropyl and R⁷ is halo;
R⁶ is C₁-C₆ alkyl, and R⁷ is C₁-C₆ alkoxy optionally substituted with one or more halo;
R⁶ is C₁-C₆ alkyl, and R⁷ is C₁-C₆ alkoxy;
R⁶ is C₁-C₆ alkyl, and R⁷ is C₁-C₆ alkoxy substituted with one or more halo;
R⁶ is halo, and R⁷ is C₁-C₆ haloalkyl;
R⁶ is halo, and R⁷ is C₁-C₆ haloalkoxy;
R⁶ is C₁-C₆ alkoxy; and R⁷ is halo;
R⁶ is C₁-C₆ alkoxy; and R⁷ is chloro;
R⁷ is C₁-C₆ alkyl, and R⁶ is C₁-C₆ alkyl optionally substituted with one or more halo;
R⁷ is C₁-C₆ alkyl, and R⁶ is C₁-C₆ alkyl substituted with one or more halo;
R⁷ is C₁-C₆ alkyl, and R⁶ is C₃-C₇ cycloalkyl;
R⁷ is C₁-C₆ alkyl, and R⁶ is halo;
R⁷ is C₁-C₆ alkyl and R⁶ is halo;
R⁷ is C₁-C₆ alkyl, and R⁶ is cyano;
R⁷ is C₃-C₇ cycloalkyl, and R⁶ is C₃-C₇ cycloalkyl;
R⁷ is C₃-C₇ cycloalkyl, and R⁶ is halo;
R⁷ is C₃-C₇ cycloalkyl and R⁶ is halo;
R⁷ is C₁-C₆ alkyl, and R⁶ is C₁-C₆ alkoxy optionally substituted with one or more halo;
R⁷ is C₁-C₆ alkyl, and R⁶ is C₁-C₆ alkoxy;
R⁷ is C₁-C₆ alkyl, and R⁶ is C₁-C₆ alkoxy substituted with one or more halo;
R⁷ is halo, and R⁶ is C₁-C₆ haloalkyl;
R⁷ is halo, and R⁶ is C₁-C₆ haloalkoxy;
R⁷ is C₁-C₆ alkoxy; and R⁶ is halo; or
R⁷ is C₁-C₆ alkoxy; and R⁶ is chloro.

In some embodiments, of the compound of formula AA,

the substituted ring B is

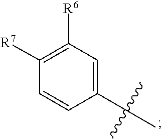

and R⁶ and R⁷ are one of the following combinations:
R⁶ is C₁-C₆ alkyl, and R⁷ is C₁-C₆ alkyl optionally substituted with one or more halo;
R⁶ is C₁-C₆ alkyl and R⁷ is C₁-C₆ alkyl;
R⁶ is C₁-C₆ alkyl, and R⁷ is C₁-C₆ alkyl substituted with one or more halo;
R⁶ is C₁-C₆ alkyl, and R⁷ is C₃-C₇ cycloalkyl;
R⁶ is C₁-C₆ alkyl, and R⁷ is halo;
R⁶ is C₁-C₆ alkyl, and R⁷ is cyano;
R⁶ is C₃-C₇ cycloalkyl, and R⁷ is C₃-C₇ cycloalkyl;
R⁶ is C₃-C₇ cycloalkyl, and R⁷ is halo;
R⁶ is cyclopropyl and R⁷ is halo;
R⁶ is C₁-C₆ alkyl, and R⁷ is C₁-C₆ alkoxy optionally substituted with one or more halo;
R⁶ is C₁-C₆ alkyl, and R⁷ is C₁-C₆ alkoxy;
R⁶ is C₁-C₆ alkyl, and R⁷ is C₁-C₆ alkoxy substituted with one or more halo;
R⁶ is halo, and R⁷ is C₁-C₆ haloalkyl;
R⁶ is halo, and R⁷ is C₁-C₆ haloalkoxy;
R⁶ is C₁-C₆ alkoxy; and R⁷ is halo;
R⁶ is C₁-C₆ alkoxy; and R⁷ is chloro;
R⁷ is C₁-C₆ alkyl, and R⁶ is C₁-C₆ alkyl optionally substituted with one or more halo;
R⁷ is C₁-C₆ alkyl, and R⁶ is C₁-C₆ alkyl substituted with one or more halo;
R⁷ is C₁-C₆ alkyl, and R⁶ is C₃-C₇ cycloalkyl;
R⁷ is C₁-C₆ alkyl, and R⁶ is halo;
R⁷ is C₁-C₆ alkyl and R⁶ is halo;
R⁷ is C₁-C₆ alkyl, and R⁶ is cyano;
R⁷ is C₃-C₇ cycloalkyl, and R⁶ is C₃-C₇ cycloalkyl;
R⁷ is C₃-C₇ cycloalkyl, and R⁶ is halo;
R⁷ is C₃-C₇ cycloalkyl and R⁶ is halo;
R⁷ is C₁-C₆ alkyl, and R⁶ is C₁-C₆ alkoxy optionally substituted with one or more halo;
R⁷ is C₁-C₆ alkyl, and R⁶ is C₁-C₆ alkoxy;
R⁷ is C₁-C₆ alkyl, and R⁶ is C₁-C₆ alkoxy substituted with one or more halo;
R⁷ is halo, and R⁶ is C₁-C₆ haloalkyl;
R⁷ is halo, and R⁶ is C₁-C₆ haloalkoxy;
R⁷ is C₁-C₆ alkoxy; and R⁶ is halo;
R⁷ is C₁-C₆ alkoxy; and R⁶ is chloro;
R⁶ and R⁷ on adjacent atoms taken together with the atoms connecting them form a C₄-C₆ aliphatic carbocyclic ring optionally substituted with one or more hydroxy, oxo, or C₁-C₆ alkyl; or
R⁶ and R⁷ on adjacent atoms taken together with the atoms connecting them form a 5-to-6-membered heterocyclic ring containing 1 heteroatom independently selected from O, N, and S, wherein the heterocyclic ring optionally substituted with one or more hydroxy, oxo, or C₁-C₆ alkyl.

In some embodiments, of the compound of formula AA,

the substituted ring B is

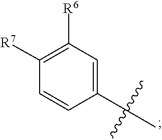

and R⁶ and R⁷ are one of the following combinations:
R⁶ is isopropyl; and R⁷ is methyl;
R⁶ is isopropyl; and R⁷ is isopropyl;
R⁶ is isopropyl; and R⁷ is trifluoromethyl;
R⁶ is isopropyl; and R⁷ is cyclopropyl;
R⁶ is isopropyl; and R⁷ is chloro;
R⁶ is isopropyl; and R⁷ is fluoro;
R⁶ is ethyl; and R⁷ is fluoro;
R⁶ is isopropyl; and R⁷ is cyano;
R⁶ is cyclopropyl; and R⁷ is cyclopropyl;
R⁶ is cyclopropyl; and R⁷ is chloro;
R⁶ is cyclopropyl; and R⁷ is fluoro;
R⁶ is isopropyl; and R⁷ is methoxy;
R⁶ is isopropyl; and R⁷ is trifluoromethoxy;
R⁶ is chloro; and R⁷ is trifluoromethyl;
R⁶ is chloro; and R⁷ is trifluoromethoxy;
R⁷ is isopropyl; and R⁶ is methyl;
R⁷ is isopropyl; and R⁶ is trifluoromethyl;
R⁷ is isopropyl; and R⁶ is cyclopropyl;
R⁷ is isopropyl; and R⁶ is chloro;
R⁷ is ethyl; and R⁶ is fluoro;
R⁷ is isopropyl; and R⁶ is cyano;
R⁷ is cyclopropyl; and R⁶ is cyclopropyl;
R⁷ is cyclopropyl; and R⁶ is chloro;
R⁷ is cyclopropyl; and R⁶ is fluoro;
R⁷ is isopropyl; and R⁶ is methoxy;
R⁷ is isopropyl; and R⁶ is trifluoromethoxy;
R⁷ is chloro; and R⁶ is trifluoromethyl;
R⁷ is chloro; and R⁶ is trifluoromethoxy;
R⁶ and R⁷ on adjacent atoms taken together with the atoms connecting them form a C₄ aliphatic carbocyclic ring;
R⁶ and R⁷ on adjacent atoms taken together with the atoms connecting them form a C₅ aliphatic carbocyclic ring;
R⁶ and R⁷ on adjacent atoms taken together with the atoms connecting them form a C₆ aliphatic carbocyclic ring;
R⁶ and R⁷ on adjacent atoms taken together with the atoms connecting them form a 5-membered heterocyclic ring containing 1 heteroatoms independently selected from O, N, and S;
R⁶ and R⁷ on adjacent atoms taken together with the atoms connecting them form a 6-membered heterocyclic ring containing 1 heteroatoms independently selected from O, N, and S; or
R⁶ and R⁷ on adjacent atoms taken together with the atoms connecting them form a C₅ aliphatic carbocyclic ring.

In some embodiments, of the compound of formula AA,

the substituted ring B is

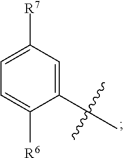

and R⁶ and R⁷ are one of the following combinations:
R⁶ is isopropyl; and R⁷ is methyl;
R⁶ is isopropyl; and R⁷ is isopropyl;
R⁶ is isopropyl; and R⁷ is trifluoromethyl;
R⁶ is isopropyl; and R⁷ is cyclopropyl;
R⁶ is isopropyl; and R⁷ is chloro;
R⁶ is isopropyl; and R⁷ is fluoro;
R⁶ is ethyl; and R⁷ is fluoro;
R⁶ is isopropyl; and R⁷ is cyano;
R⁶ is cyclopropyl; and R⁷ is cyclopropyl;
R⁶ is cyclopropyl; and R⁷ is chloro;
R⁶ is cyclopropyl; and R⁷ is fluoro;
R⁶ is isopropyl; and R⁷ is methoxy;
R⁶ is isopropyl; and R⁷ is trifluoromethoxy;
R⁶ is chloro; and R⁷ is trifluoromethyl;
R⁶ is chloro; and R⁷ is trifluoromethoxy;
R⁷ is isopropyl; and R⁶ is methyl;
R⁷ is isopropyl; and R⁶ is trifluoromethyl;
R⁷ is isopropyl; and R⁶ is cyclopropyl;
R⁷ is isopropyl; and R⁶ is chloro;
R⁷ is ethyl; and R⁶ is fluoro;
R⁷ is isopropyl; and R⁶ is cyano;
R⁷ is cyclopropyl; and R⁶ is cyclopropyl;
R⁷ is cyclopropyl; and R⁶ is chloro;
R⁷ is cyclopropyl; and R⁶ is fluoro;
R⁷ is isopropyl; and R⁶ is methoxy;
R⁷ is isopropyl; and R⁶ is trifluoromethoxy;
R⁷ is chloro; and R⁶ is trifluoromethyl; or
R⁷ is chloro; and R⁶ is trifluoromethoxy.

In some embodiments, of the compound of formula AA,

the substituted ring B is

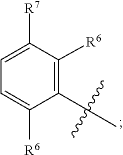

and R⁶ and R⁷ are one of the following combinations:
each R⁶ is independently C₁-C₆ alkyl, and R⁷ is C₁-C₆ alkyl optionally substituted with one or more halo;
each R⁶ is independently C₁-C₆ alkyl and R⁷ is C₁-C₆ alkyl;
each R⁶ is independently C₁-C₆ alkyl, and R⁷ is C₁-C₆ alkyl substituted with one or more halo;
each R⁶ is independently C₁-C₆ alkyl, and R⁷ is C₃-C₇ cycloalkyl;
each R⁶ is independently C₁-C₆ alkyl, and R⁷ is halo;
each R⁶ is independently C₁-C₆ alkyl, and R⁷ is cyano;
each R⁶ is independently C₃-C₇ cycloalkyl, and R⁷ is C₃-C₇ cycloalkyl;
each R⁶ is independently C₃-C₇ cycloalkyl, and R⁷ is halo;
each R⁶ is independently cyclopropyl and R⁷ is halo;
each R⁶ is independently C₁-C₆ alkyl, and R⁷ is C₁-C₆ alkoxy optionally substituted with one or more halo;
each R⁶ is independently C₁-C₆ alkyl, and R⁷ is C₁-C₆ alkoxy;
each R⁶ is independently C₁-C₆ alkyl, and R⁷ is C₁-C₆ alkoxy substituted with one or more halo;
each R⁶ is independently halo, and R⁷ is C₁-C₆ haloalkyl;
each R⁶ is independently halo, and R⁷ is C₁-C₆ haloalkoxy;
each R⁶ is independently C₁-C₆ alkoxy; and R⁷ is halo;
each R⁶ is independently C₁-C₆ alkoxy; and R⁷ is chloro;
R⁷ is C₁-C₆ alkyl, and each R⁶ is independently C₁-C₆ alkyl optionally substituted with one or more halo;
R⁷ is C₁-C₆ alkyl, and each R⁶ is independently C₁-C₆ alkyl substituted with one or more halo;
R⁷ is C₁-C₆ alkyl, and each R⁶ is independently C₃-C₇ cycloalkyl;
R⁷ is C₁-C₆ alkyl, and each R⁶ is independently halo;
R⁷ is C₁-C₆ alkyl and each R⁶ is independently halo;
R⁷ is C₁-C₆ alkyl, and R⁶ is cyano;
R⁷ is C₃-C₇ cycloalkyl, and each R⁶ is independently C₃-C₇ cycloalkyl;
R⁷ is C₃-C₇ cycloalkyl, and each R⁶ is independently halo;
R⁷ is C₃-C₇ cycloalkyl and each R⁶ is independently halo;
R⁷ is C₁-C₆ alkyl, and each R⁶ is independently C₁-C₆ alkoxy optionally substituted with one or more halo;
R⁷ is C₁-C₆ alkyl, and each R⁶ is independently C₁-C₆ alkoxy;
R⁷ is C₁-C₆ alkyl, and each R⁶ is independently C₁-C₆ alkoxy substituted with one or more halo;
R⁷ is halo, and each R⁶ is independently C₁-C₆ haloalkyl;
R⁷ is halo, and each R⁶ is independently C₁-C₆ haloalkoxy;
R⁷ is C₁-C₆ alkoxy; and each R⁶ is independently halo;
R⁷ is C₁-C₆ alkoxy; and R⁶ is chloro;
R⁶ and R⁷ on adjacent atoms taken together with the atoms connecting them form a C₅ aliphatic carbocyclic ring;
R⁶ and R⁷ on adjacent atoms taken together with the atoms connecting them form a C₄-C₆ aliphatic carbocyclic ring optionally substituted with one or more hydroxy, oxo, or C₁-C₆ alkyl; and one R⁶ is halo or cyano; or
R⁶ and R⁷ on adjacent atoms taken together with the atoms connecting them form a 5-to-6-membered heterocyclic ring containing 1 heteroatom independently selected from O, N, and S, wherein the heterocyclic ring optionally substituted with one or more hydroxy, oxo, or C₁-C₆ alkyl; and one R⁶ is halo or cyano.

In some embodiments, of the compound of formula AA,

the substituted ring B is

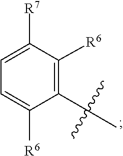

and R⁶ and R⁷ are one of the following combinations:
each R⁶ is isopropyl; and R⁷ is methyl;
each R⁶ is isopropyl; and R⁷ is isopropyl;
each R⁶ is isopropyl; and R⁷ is trifluoromethyl;
each R⁶ is isopropyl; and R⁷ is cyclopropyl;
each R⁶ is isopropyl; and R⁷ is chloro;
each R⁶ is isopropyl; and R⁷ is fluoro;
each R⁶ is ethyl; and R⁷ is fluoro;
each R⁶ is isopropyl; and R⁷ is cyano;
each R⁶ is cyclopropyl; and R⁷ is cyclopropyl;
each R⁶ is cyclopropyl; and R⁷ is chloro;
each R⁶ is cyclopropyl; and R⁷ is fluoro;
each R⁶ is isopropyl; and R⁷ is methoxy;
each R⁶ is isopropyl; and R⁷ is trifluoromethoxy;
each R⁶ is chloro; and R⁷ is trifluoromethyl;
each R⁶ is chloro; and R⁷ is trifluoromethoxy;
R⁷ is isopropyl; and each R⁶ is methyl;
R⁷ is isopropyl; and each R⁶ is trifluoromethyl;
R⁷ is isopropyl; and each R⁶ is cyclopropyl;
R⁷ is isopropyl; and each R⁶ is chloro;
R⁷ is ethyl; and each R⁶ is fluoro;
R⁷ is isopropyl; and each R⁶ is cyano;
R⁷ is cyclopropyl; and each R⁶ is cyclopropyl;
R⁷ is cyclopropyl; and each R⁶ is chloro;
R⁷ is cyclopropyl; and each R⁶ is fluoro;
R⁷ is isopropyl; and each R⁶ is methoxy;
R⁷ is isopropyl; and each R⁶ is trifluoromethoxy;
R⁷ is chloro; and each R⁶ is trifluoromethyl;
R⁷ is chloro; and each R⁶ is trifluoromethoxy;
one R⁶ is isopropyl; the other R⁶ is trifluoromethyl; and R⁷ is chloro;
R⁶ and R⁷ on adjacent atoms taken together with the atoms connecting them form a C₄ aliphatic carbocyclic ring; and one R⁶ is fluoro, chloro, or cyano;
R⁶ and R⁷ on adjacent atoms taken together with the atoms connecting them form a C₅ aliphatic carbocyclic ring; and one R⁶ is fluoro, chloro, or cyano;
R⁶ and R⁷ on adjacent atoms taken together with the atoms connecting them form a C₆ aliphatic carbocyclic ring; and one R⁶ is fluoro, chloro, or cyano;
R⁶ and R⁷ on adjacent atoms taken together with the atoms connecting them form a 5-membered heterocyclic ring containing 1 heteroatoms independently selected from O, N, and S; and one R⁶ is fluoro, chloro, or cyano; or
R⁶ and R⁷ on adjacent atoms taken together with the atoms connecting them form a 6-membered heterocyclic ring containing 1 heteroatoms independently selected from O, N, and S; and one R⁶ is fluoro, chloro, or cyano.

In some embodiments, of the compound of formula AA,

the substituted ring B is

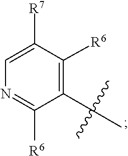

and R⁶ and R⁷ are one of the following combinations:
each R⁶ is independently C₁-C₆ alkyl, and R⁷ is C₁-C₆ alkyl optionally substituted with one or more halo;
each R⁶ is independently C₁-C₆ alkyl and R⁷ is C₁-C₆ alkyl;
each R⁶ is independently C₁-C₆ alkyl, and R⁷ is C₁-C₆ alkyl substituted with one or more halo;
each R⁶ is independently C₁-C₆ alkyl, and R⁷ is C₃-C₇ cycloalkyl;
each R⁶ is independently C₁-C₆ alkyl, and R⁷ is halo;
each R⁶ is independently C₁-C₆ alkyl, and R⁷ is cyano;
each R⁶ is independently C₃-C₇ cycloalkyl, and R⁷ is C₃-C₇ cycloalkyl;
each R⁶ is independently C₃-C₇ cycloalkyl, and R⁷ is halo;
each R⁶ is independently cyclopropyl and R⁷ is halo;
each R⁶ is independently C₁-C₆ alkyl, and R⁷ is C₁-C₆ alkoxy optionally substituted with one or more halo;
each R⁶ is independently C₁-C₆ alkyl, and R⁷ is C₁-C₆ alkoxy;
each R⁶ is independently C₁-C₆ alkyl, and R⁷ is C₁-C₆ alkoxy substituted with one or more halo;
each R⁶ is independently halo, and R⁷ is C₁-C₆ haloalkyl;
each R⁶ is independently halo, and R⁷ is C₁-C₆ haloalkoxy;
each R⁶ is independently C₁-C₆ alkoxy; and R⁷ is halo;
each R⁶ is independently C₁-C₆ alkoxy; and R⁷ is chloro;
R⁷ is C₁-C₆ alkyl, and each R⁶ is independently C₁-C₆ alkyl optionally substituted with one or more halo;
R⁷ is C₁-C₆ alkyl, and each R⁶ is independently C₁-C₆ alkyl substituted with one or more halo;
R⁷ is C₁-C₆ alkyl, and each R⁶ is independently C₃-C₇ cycloalkyl;
R⁷ is C₁-C₆ alkyl, and each R⁶ is independently halo;
R⁷ is C₁-C₆ alkyl and each R⁶ is independently halo;
R⁷ is C₁-C₆ alkyl, and R⁶ is cyano;
R⁷ is C₃-C₇ cycloalkyl, and each R⁶ is independently C₃-C₇ cycloalkyl;
R⁷ is C₃-C₇ cycloalkyl, and each R⁶ is independently halo;
R⁷ is C₃-C₇ cycloalkyl and each R⁶ is independently halo;
R⁷ is C₁-C₆ alkyl, and each R⁶ is independently C₁-C₆ alkoxy optionally substituted with one or more halo;
R⁷ is C₁-C₆ alkyl, and each R⁶ is independently C₁-C₆ alkoxy;
R⁷ is C₁-C₆ alkyl, and each R⁶ is independently C₁-C₆ alkoxy substituted with one or more halo;
R⁷ is halo, and each R⁶ is independently C₁-C₆ haloalkyl;
R⁷ is halo, and each R⁶ is independently C₁-C₆ haloalkoxy;
R⁷ is C₁-C₆ alkoxy; and each R⁶ is independently halo; or
R⁷ is C₁-C₆ alkoxy; and R⁶ is chloro.

In some embodiments, of the compound of formula AA,

the substituted ring B is

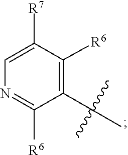

and R⁶ and R⁷ are one of the following combinations:
each R⁶ is isopropyl; and R⁷ is methyl;
each R⁶ is isopropyl; and R⁷ is isopropyl;
each R⁶ is isopropyl; and R⁷ is trifluoromethyl;
each R⁶ is isopropyl; and R⁷ is cyclopropyl;
each R⁶ is isopropyl; and R⁷ is chloro;
each R⁶ is isopropyl; and R⁷ is fluoro;
each R⁶ is ethyl; and R⁷ is fluoro;
each R⁶ is isopropyl; and R⁷ is cyano;
each R⁶ is cyclopropyl; and R⁷ is cyclopropyl;
each R⁶ is cyclopropyl; and R⁷ is chloro;
each R⁶ is cyclopropyl; and R⁷ is fluoro;
each R⁶ is isopropyl; and R⁷ is methoxy;
each R⁶ is isopropyl; and R⁷ is trifluoromethoxy;
each R⁶ is chloro; and R⁷ is trifluoromethyl;
each R⁶ is chloro; and R⁷ is trifluoromethoxy;
R⁷ is isopropyl; and each R⁶ is methyl;
R⁷ is isopropyl; and each R⁶ is trifluoromethyl;
R⁷ is isopropyl; and each R⁶ is cyclopropyl;
R⁷ is isopropyl; and each R⁶ is chloro;
R⁷ is ethyl; and each R⁶ is fluoro;
R⁷ is isopropyl; and each R⁶ is cyano;
R⁷ is cyclopropyl; and each R⁶ is cyclopropyl;
R⁷ is cyclopropyl; and each R⁶ is chloro;
R⁷ is cyclopropyl; and each R⁶ is fluoro;
R⁷ is isopropyl; and each R⁶ is methoxy;
R⁷ is isopropyl; and each R⁶ is trifluoromethoxy;
R⁷ is chloro; and each R⁶ is trifluoromethyl;
R⁷ is chloro; and each R⁶ is trifluoromethoxy; or
one R⁶ is isopropyl; the other R⁶ is trifluoromethyl; and R⁷ is chloro.

In some embodiments, of the compound of formula AA,

the substituted ring B is

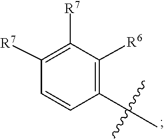

and R⁶ and R⁷ are one of the following combinations:
R⁶ is C₁-C₆ alkyl, and each R⁷ is independently C₁-C₆ alkyl optionally substituted with one or more halo;
R⁶ is C₁-C₆ alkyl and each R⁷ is independently C₁-C₆ alkyl;
R⁶ is C₁-C₆ alkyl, and each R⁷ is independently C₁-C₆ alkyl substituted with one or more halo;
R⁶ is C₁-C₆ alkyl, and each R⁷ is independently C₃-C₇ cycloalkyl;
R⁶ is C₁-C₆ alkyl, and each R⁷ is independently halo;
R⁶ is C₁-C₆ alkyl, and R⁷ is cyano;
R⁶ is C₃-C₇ cycloalkyl, and each R⁷ is independently C₃-C₇ cycloalkyl;
R⁶ is C₃-C₇ cycloalkyl, and each R⁷ is independently halo;
R⁶ is cyclopropyl and each R⁷ is independently halo;
R⁶ is C₁-C₆ alkyl, and each R⁷ is independently C₁-C₆ alkoxy optionally substituted with one or more halo;
R⁶ is C₁-C₆ alkyl, and each R⁷ is independently C₁-C₆ alkoxy;
R⁶ is C₁-C₆ alkyl, and each R⁷ is independently C₁-C₆ alkoxy substituted with one or more halo;
R⁶ is halo, and each R⁷ is independently C₁-C₆ haloalkyl;
R⁶ is halo, and each R⁷ is independently C₁-C₆ haloalkoxy;
R⁶ is C₁-C₆ alkoxy; and each R⁷ is independently halo;
R⁶ is C₁-C₆ alkoxy; and R⁷ is chloro;
each R⁷ is independently C₁-C₆ alkyl, and R⁶ is C₁-C₆ alkyl optionally substituted with one or more halo;
each R⁷ is independently C₁-C₆ alkyl, and R⁶ is C₁-C₆ alkyl substituted with one or more halo;
each R⁷ is independently C₁-C₆ alkyl, and R⁶ is C₃-C₇ cycloalkyl;
each R⁷ is independently C₁-C₆ alkyl, and R⁶ is halo;
each R⁷ is independently C₁-C₆ alkyl and R⁶ is halo;
each R⁷ is independently C₁-C₆ alkyl, and R⁶ is cyano;
each R⁷ is independently C₃-C₇ cycloalkyl, and R⁶ is C₃-C₇ cycloalkyl.
each R⁷ is independently C₃-C₇ cycloalkyl, and R⁶ is halo;
each R⁷ is independently C₃-C₇ cycloalkyl and R⁶ is halo;
each R⁷ is independently C₁-C₆ alkyl, and R⁶ is C₁-C₆ alkoxy optionally substituted with one or more halo;
each R⁷ is independently C₁-C₆ alkyl, and R⁶ is C₁-C₆ alkoxy;
each R⁷ is independently C₁-C₆ alkyl, and R⁶ is C₁-C₆ alkoxy substituted with one or more halo;
each R⁷ is independently halo, and R⁶ is C₁-C₆ haloalkyl;
each R⁷ is independently halo, and R⁶ is C₁-C₆ haloalkoxy;
each R⁷ is independently C₁-C₆ alkoxy; and R⁶ is halo;
each R⁷ is independently C₁-C₆ alkoxy; and R⁶ is chloro;
R⁶ and R⁷ on adjacent atoms taken together with the atoms connecting them form a C₅ aliphatic carbocyclic ring;
R⁶ and R⁷ on adjacent atoms taken together with the atoms connecting them form a C₄-C₆ aliphatic carbocyclic ring optionally substituted with one or more hydroxy, oxo, or C₁-C₆ alkyl; or
R⁶ and R⁷ on adjacent atoms taken together with the atoms connecting them form a 5-to-6-membered heterocyclic ring containing 1 heteroatom independently selected from O, N, and S, wherein the heterocyclic ring optionally substituted with one or more hydroxy, oxo, or C₁-C₆ alkyl.

In some embodiments, of the compound of formula AA,

the substituted ring B is

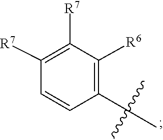

and R⁶ and R⁷ are one of the following combinations:
R⁶ is isopropyl; and each R⁷ is methyl;
R⁶ is isopropyl; and each R⁷ is isopropyl;
R⁶ is isopropyl; and each R⁷ is trifluoromethyl;
R⁶ is isopropyl; and each R⁷ is cyclopropyl;
R⁶ is isopropyl; and each R⁷ is chloro;
R⁶ is isopropyl; and each R⁷ is fluoro;
R⁶ is ethyl; and each R⁷ is fluoro;
R⁶ is isopropyl; and each R⁷ is cyano;
R⁶ is cyclopropyl; and each R⁷ is cyclopropyl;
R⁶ is cyclopropyl; and each R⁷ is chloro;
R⁶ is cyclopropyl; and each R⁷ is fluoro;
R⁶ is isopropyl; and R⁷ is methoxy;
R⁶ is isopropyl; and each R⁷ is trifluoromethoxy;
R⁶ is chloro; and each R⁷ is trifluoromethyl;
R⁶ is chloro; and each R⁷ is trifluoromethoxy;
each R⁷ is isopropyl; and R⁶ is methyl;
each R⁷ is isopropyl; and R⁶ is trifluoromethyl;
each R⁷ is isopropyl; and R⁶ is cyclopropyl;
each R⁷ is isopropyl; and R⁶ is chloro;
each R⁷ is ethyl; and R⁶ is fluoro;
each R⁷ is isopropyl; and R⁶ is cyano;
each R⁷ is cyclopropyl; and R⁶ is cyclopropyl;
each R⁷ is cyclopropyl; and R⁶ is chloro;
each R⁷ is cyclopropyl; and R⁶ is fluoro;
each R⁷ is isopropyl; and R⁶ is methoxy;
each R⁷ is isopropyl; and R⁶ is trifluoromethoxy;
each R⁷ is chloro; and R⁶ is trifluoromethyl;
each R⁷ is chloro; and R⁶ is trifluoromethoxy;
R⁶ and R⁷ on adjacent atoms taken together with the atoms connecting them form a C₅ aliphatic carbocyclic ring; and one R⁷ is fluoro, chloro, or cyano;
R⁶ and R⁷ on adjacent atoms taken together with the atoms connecting them form a C₄ aliphatic carbocyclic ring; and one R⁷ is fluoro, chloro, or cyano;
R⁶ and R⁷ on adjacent atoms taken together with the atoms connecting them form a C₆ aliphatic carbocyclic ring; and one R⁷ is fluoro, chloro, or cyano;
R⁶ and R⁷ on adjacent atoms taken together with the atoms connecting them form a 5-membered heterocyclic ring containing 1 heteroatoms independently selected from O, N, and S; and one R⁷ is fluoro, chloro, or cyano; or
R⁶ and R⁷ on adjacent atoms taken together with the atoms connecting them form a 6-membered heterocyclic ring containing 1 heteroatoms independently selected from O, N, and S; and one R⁷ is fluoro, chloro, or cyano.

In some embodiments, of the compound of formula AA,

the substituted ring B is

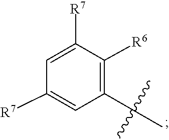

and R⁶ and Ware one of the following combinations:
R⁶ is C₁-C₆ alkyl, and each R⁷ is independently C₁-C₆ alkyl optionally substituted with one or more halo;
R⁶ is C₁-C₆ alkyl and each R⁷ is independently C₁-C₆ alkyl;
R⁶ is C₁-C₆ alkyl, and each R⁷ is independently C₁-C₆ alkyl substituted with one or more halo;
R⁶ is C₁-C₆ alkyl, and each R⁷ is independently C₃-C₇ cycloalkyl;
R⁶ is C₁-C₆ alkyl, and each R⁷ is independently halo;
R⁶ is C₁-C₆ alkyl, and R⁷ is cyano;
R⁶ is C₃-C₇ cycloalkyl, and each R⁷ is independently C₃-C₇ cycloalkyl;
R⁶ is C₃-C₇ cycloalkyl, and each R⁷ is independently halo;
R⁶ is cyclopropyl and each R⁷ is independently halo;
R⁶ is C₁-C₆ alkyl, and each R⁷ is independently C₁-C₆ alkoxy optionally substituted with one or more halo;
R⁶ is C₁-C₆ alkyl, and each R⁷ is independently C₁-C₆ alkoxy;
R⁶ is C₁-C₆ alkyl, and each R⁷ is independently C₁-C₆ alkoxy substituted with one or more halo;
R⁶ is halo, and each R⁷ is independently C₁-C₆ haloalkyl;
R⁶ is halo, and each R⁷ is independently C₁-C₆ haloalkoxy;
R⁶ is C₁-C₆ alkoxy; and each R⁷ is independently halo;
R⁶ is C₁-C₆ alkoxy; and R⁷ is chloro;
each R⁷ is independently C₁-C₆ alkyl, and R⁶ is C₁-C₆ alkyl optionally substituted with one or more halo;
each R⁷ is independently C₁-C₆ alkyl, and R⁶ is C₁-C₆ alkyl substituted with one or more halo;
each R⁷ is independently C₁-C₆ alkyl, and R⁶ is C₃-C₇ cycloalkyl;
each R⁷ is independently C₁-C₆ alkyl, and R⁶ is halo;
each R⁷ is independently C₁-C₆ alkyl and R⁶ is halo;
each R⁷ is independently C₁-C₆ alkyl, and R⁶ is cyano;
each R⁷ is independently C₃-C₇ cycloalkyl, and R⁶ is C₃-C₇ cycloalkyl;
each R⁷ is independently C₃-C₇ cycloalkyl, and R⁶ is halo;
each R⁷ is independently C₃-C₇ cycloalkyl and R⁶ is halo;
each R⁷ is independently C₁-C₆ alkyl, and R⁶ is C₁-C₆ alkoxy optionally substituted with one or more halo;
each R⁷ is independently C₁-C₆ alkyl, and R⁶ is C₁-C₆ alkoxy;
each R⁷ is independently C₁-C₆ alkyl, and R⁶ is C₁-C₆ alkoxy substituted with one or more halo;
each R⁷ is independently halo, and R⁶ is C₁-C₆ haloalkyl;
each R⁷ is independently halo, and R⁶ is C₁-C₆ haloalkoxy;
each R⁷ is independently C₁-C₆ alkoxy; and R⁶ is halo;
each R⁶ is independently C₁-C₆ alkoxy; and R⁶ is chloro;
R⁶ and R⁷ on adjacent atoms taken together with the atoms connecting them form a C₅ aliphatic carbocyclic ring;
R⁶ and R⁷ on adjacent atoms taken together with the atoms connecting them form a C₄-C₆ aliphatic carbocyclic ring optionally substituted with one or more hydroxy, oxo, or C₁-C₆ alkyl; or
R⁶ and R⁷ on adjacent atoms taken together with the atoms connecting them form a 5-to-6-membered heterocyclic ring containing 1 heteroatom independently selected from O, N, and S, wherein the heterocyclic ring optionally substituted with one or more hydroxy, oxo, or C₁-C₆ alkyl.

In some embodiments, of the compound of formula AA,

the substituted ring B is

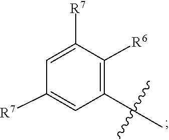

and R⁶ and R⁷ are one of the following combinations:
R⁶ is isopropyl; and each R⁷ is methyl;
R⁶ is isopropyl; and each R⁷ is isopropyl;
R⁶ is isopropyl; and each R⁷ is trifluoromethyl;
R⁶ is isopropyl; and each R⁷ is cyclopropyl;
R⁶ is isopropyl; and each R⁷ is chloro;
R⁶ is isopropyl; and each R⁷ is fluoro;
R⁶ is ethyl; and each R⁷ is fluoro;
R⁶ is isopropyl; and each R⁷ is cyano;
R⁶ is cyclopropyl; and each R⁷ is cyclopropyl;
R⁶ is cyclopropyl; and each R⁷ is chloro;
R⁶ is cyclopropyl; and each R⁷ is fluoro;
R⁶ is isopropyl; and each R⁷ is methoxy;
R⁶ is isopropyl; and each R⁷ is trifluoromethoxy;
R⁶ is chloro; and each R⁷ is trifluoromethyl;
R⁶ is chloro; and each R⁷ is trifluoromethoxy;
each R⁷ is isopropyl; and R⁶ is methyl;
each R⁷ is isopropyl; and R⁶ is trifluoromethyl;
each R⁷ is isopropyl; and R⁶ is cyclopropyl;
each R⁷ is isopropyl; and R⁶ is chloro;
each R⁷ is ethyl; and R⁶ is fluoro;
each R⁷ is isopropyl; and R⁶ is cyano;
each R⁷ is cyclopropyl; and R⁶ is cyclopropyl;
each R⁷ is cyclopropyl; and R⁶ is chloro;
each R⁷ is cyclopropyl; and R⁶ is fluoro;
each R⁷ is isopropyl; and R⁶ is methoxy;
each R⁷ is isopropyl; and R⁶ is trifluoromethoxy;
each R⁷ is chloro; and R⁶ is trifluoromethyl;
each R⁷ is chloro; and R⁶ is trifluoromethoxy;
R⁶ and R⁷ on adjacent atoms taken together with the atoms connecting them form a C₅ aliphatic carbocyclic ring; and one R⁷ is fluoro, chloro, or cyano;
R⁶ and R⁷ on adjacent atoms taken together with the atoms connecting them form a C₄ aliphatic carbocyclic ring; and one R⁷ is fluoro, chloro, or cyano;
R⁶ and R⁷ on adjacent atoms taken together with the atoms connecting them form a C₆ aliphatic carbocyclic ring; and one R⁷ is fluoro, chloro, or cyano;
R⁶ and R⁷ on adjacent atoms taken together with the atoms connecting them form a 5-membered heterocyclic ring containing 1 heteroatoms independently selected from O, N, and S; and one R⁷ is fluoro, chloro, or cyano; or
R⁶ and R⁷ on adjacent atoms taken together with the atoms connecting them form a 6-membered heterocyclic ring containing 1 heteroatoms independently selected from O, N, and S; and one R⁷ is fluoro, chloro, or cyano.

In some embodiments, of the compound of formula AA,

the substituted ring B is

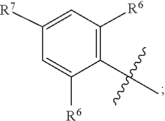

and R⁶ and R⁷ are one of the following combinations:
each R⁶ is independently C₁-C₆ alkyl, and R⁷ is C₁-C₆ alkyl optionally substituted with one or more halo;
each R⁶ is independently C₁-C₆ alkyl and R⁷ is C₁-C₆ alkyl;
each R⁶ is independently C₁-C₆ alkyl, and R⁷ is C₁-C₆ alkyl substituted with one or more halo;
each R⁶ is independently C₁-C₆ alkyl, and R⁷ is C₃-C₇ cycloalkyl;
each R⁶ is independently C₁-C₆ alkyl, and R⁷ is halo;
each R⁶ is independently C₁-C₆ alkyl, and R⁷ is cyano;
each R⁶ is independently C₃-C₇ cycloalkyl, and R⁷ is C₃-C₇ cycloalkyl;
each R⁶ is independently C₃-C₇ cycloalkyl, and R⁷ is halo;
each R⁶ is independently cyclopropyl and R⁷ is halo;
each R⁶ is independently C₁-C₆ alkyl, and R⁷ is C₁-C₆ alkoxy optionally substituted with one or more halo;
each R⁶ is independently C₁-C₆ alkyl, and R⁷ is C₁-C₆ alkoxy;
each R⁶ is independently C₁-C₆ alkyl, and R⁷ is C₁-C₆ alkoxy substituted with one or more halo;
each R⁶ is independently halo, and R⁷ is C₁-C₆ haloalkyl;
each R⁶ is independently halo, and R⁷ is C₁-C₆ haloalkoxy;
each R⁶ is independently C₁-C₆ alkoxy; and R⁷ is halo;
each R⁶ is independently C₁-C₆ alkoxy; and R⁷ is chloro;
R⁷ is C₁-C₆ alkyl, and each R⁶ is independently C₁-C₆ alkyl optionally substituted with one or more halo;
R⁷ is C₁-C₆ alkyl, and each R⁶ is independently C₁-C₆ alkyl substituted with one or more halo;
R⁷ is C₁-C₆ alkyl, and each R⁶ is independently C₃-C₇ cycloalkyl;
R⁷ is C₁-C₆ alkyl, and each R⁶ is independently halo;
R⁷ is C₁-C₆ alkyl and each R⁶ is independently halo;
R⁷ is C₁-C₆ alkyl, and R⁶ is cyano;
R⁷ is C₃-C₇ cycloalkyl, and each R⁶ is independently C₃-C₇ cycloalkyl;
R⁷ is C₃-C₇ cycloalkyl, and each R⁶ is independently halo;
R⁷ is C₃-C₇ cycloalkyl and each R⁶ is independently halo;
R⁷ is C₁-C₆ alkyl, and each R⁶ is independently C₁-C₆ alkoxy optionally substituted with one or more halo;
R⁷ is C₁-C₆ alkyl, and each R⁶ is independently C₁-C₆ alkoxy;
R⁷ is C₁-C₆ alkyl, and each R⁶ is independently C₁-C₆ alkoxy substituted with one or more halo;
R⁷ is halo, and each R⁶ is independently C₁-C₆ haloalkyl;
R⁷ is halo, and each R⁶ is independently C₁-C₆ haloalkoxy;
R⁷ is C₁-C₆ alkoxy; and each R⁶ is independently halo; or
R⁷ is C₁-C₆ alkoxy; and R⁶ is chloro.

In some embodiments, of the compound of formula AA,

the substituted ring B is

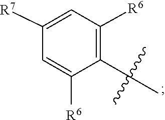

and R⁶ and R⁷ are one of the following combinations:
each R⁶ is isopropyl; and R⁷ is methyl;
each R⁶ is isopropyl; and R⁷ is isopropyl;
each R⁶ is isopropyl; and R⁷ is trifluoromethyl;
each R⁶ is isopropyl; and R⁷ is cyclopropyl;
each R⁶ is isopropyl; and R⁷ is chloro;
each R⁶ is isopropyl; and R⁷ is fluoro;
each R⁶ is ethyl; and R⁷ is fluoro;
each R⁶ is isopropyl; and R⁷ is cyano;
each R⁶ is cyclopropyl; and R⁷ is cyclopropyl;
each R⁶ is cyclopropyl; and R⁷ is chloro;
each R⁶ is cyclopropyl; and R⁷ is fluoro;
each R⁶ is isopropyl; and R⁷ is methoxy;
each R⁶ is isopropyl; and R⁷ is trifluoromethoxy;
each R⁶ is chloro; and R⁷ is trifluoromethyl;
each R⁶ is chloro; and R⁷ is trifluoromethoxy;
R⁷ is isopropyl; and each R⁶ is methyl;
R⁷ is isopropyl; and each R⁶ is trifluoromethyl;
R⁷ is isopropyl; and each R⁶ is cyclopropyl;
R⁷ is isopropyl; and each R⁶ is chloro;
R⁷ is ethyl; and each R⁶ is fluoro;
R⁷ is isopropyl; and each R⁶ is cyano;
R⁷ is cyclopropyl; and each R⁶ is cyclopropyl;
R⁷ is cyclopropyl; and each R⁶ is chloro;
R⁷ is cyclopropyl; and each R⁶ is fluoro;
R⁷ is isopropyl; and each R⁶ is methoxy;
R⁷ is isopropyl; and each R⁶ is trifluoromethoxy;
R⁷ is chloro; and each R⁶ is trifluoromethyl;
R⁷ is chloro; and each R⁶ is trifluoromethoxy; or
one R⁶ is isopropyl; the other R⁶ is trifluoromethyl; and R⁷ is chloro.

In some embodiments, of the compound of formula AA,

the substituted ring B is

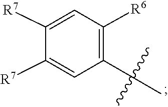

and R⁶ and R⁷ are one of the following combinations:
R⁶ is C₁-C₆ alkyl, and each R⁷ is independently C₁-C₆ alkyl optionally substituted with one or more halo;
R⁶ is C₁-C₆ alkyl and each R⁷ is independently C₁-C₆ alkyl;
R⁶ is C₁-C₆ alkyl, and each R⁷ is independently C₁-C₆ alkyl substituted with one or more halo;
R⁶ is C₁-C₆ alkyl, and each R⁷ is independently C₃-C₇ cycloalkyl;
R⁶ is C₁-C₆ alkyl, and each R⁷ is independently halo;
R⁶ is C₁-C₆ alkyl, and R⁷ is cyano;
R⁶ is C₃-C₇ cycloalkyl, and each R⁷ is independently C₃-C₇ cycloalkyl;
R⁶ is C₃-C₇ cycloalkyl, and each R⁷ is independently halo;
R⁶ is cyclopropyl and each R⁷ is independently halo;
R⁶ is C₁-C₆ alkyl, and each R⁷ is independently C₁-C₆ alkoxy optionally substituted with one or more halo;
R⁶ is C₁-C₆ alkyl, and each R⁷ is independently C₁-C₆ alkoxy;
R⁶ is C₁-C₆ alkyl, and each R⁷ is independently C₁-C₆ alkoxy substituted with one or more halo;
R⁶ is halo, and each R⁷ is independently C₁-C₆ haloalkyl;
R⁶ is halo, and each R⁷ is independently C₁-C₆ haloalkoxy;
R⁶ is C₁-C₆ alkoxy; and each R⁷ is independently halo;
R⁶ is C₁-C₆ alkoxy; and R⁷ is chloro;
each R⁷ is independently C₁-C₆ alkyl, and R⁶ is C₁-C₆ alkyl optionally substituted with one or more halo;
each R⁷ is independently C₁-C₆ alkyl, and R⁶ is C₁-C₆ alkyl substituted with one or more halo;
each R⁷ is independently C₁-C₆ alkyl, and R⁶ is C₃-C₇ cycloalkyl;
each R⁷ is independently C₁-C₆ alkyl, and R⁶ is halo;
each R⁷ is independently C₁-C₆ alkyl and R⁶ is halo;
each R⁷ is independently C₁-C₆ alkyl, and R⁶ is cyano;
each R⁷ is independently C₃-C₇ cycloalkyl, and R⁶ is C₃-C₇ cycloalkyl;
each R⁷ is independently C₃-C₇ cycloalkyl, and R⁶ is halo;
each R⁷ is independently C₃-C₇ cycloalkyl and R⁶ is halo;
each R⁷ is independently C₁-C₆ alkyl, and R⁶ is C₁-C₆ alkoxy optionally substituted with one or more halo;
each R⁷ is independently C₁-C₆ alkyl, and R⁶ is C₁-C₆ alkoxy;
each R⁷ is independently C₁-C₆ alkyl, and R⁶ is C₁-C₆ alkoxy substituted with one or more halo;
each R⁷ is independently halo, and R⁶ is C₁-C₆ haloalkyl;
each R⁷ is independently halo, and R⁶ is C₁-C₆ haloalkoxy;
each R⁷ is independently C₁-C₆ alkoxy; and R⁶ is halo; or
each R⁷ is independently C₁-C₆ alkoxy; and R⁶ is chloro.

In some embodiments, of the compound of formula AA,

the substituted ring B is

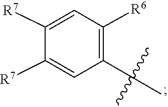

and R⁶ and R⁷ are one of the following combinations:
R⁶ is isopropyl; and each R⁷ is methyl;
R⁶ is isopropyl; and each R⁷ is isopropyl;
R⁶ is isopropyl; and each R⁷ is trifluoromethyl;
R⁶ is isopropyl; and each R⁷ is cyclopropyl;
R⁶ is isopropyl; and each R⁷ is chloro;
R⁶ is isopropyl; and each R⁷ is fluoro;
R⁶ is ethyl; and each R⁷ is fluoro;
R⁶ is isopropyl; and each R⁷ is cyano;
R⁶ is cyclopropyl; and each R⁷ is cyclopropyl;
R⁶ is cyclopropyl; and each R⁷ is chloro;
R⁶ is cyclopropyl; and each R⁷ is fluoro;
R⁶ is isopropyl; and each R⁷ is methoxy;
R⁶ is isopropyl; and each R⁷ is trifluoromethoxy;
R⁶ is chloro; and each R⁷ is trifluoromethyl;
R⁶ is chloro; and each R⁷ is trifluoromethoxy;
each R⁷ is isopropyl; and R⁶ is methyl;
each R⁷ is isopropyl; and R⁶ is trifluoromethyl;
each R⁷ is isopropyl; and R⁶ is cyclopropyl;
each R⁷ is isopropyl; and R⁶ is chloro;
each R⁷ is ethyl; and R⁶ is fluoro;
each R⁷ is isopropyl; and R⁶ is cyano;
each R⁷ is cyclopropyl; and R⁶ is cyclopropyl;
each R⁷ is cyclopropyl; and R⁶ is chloro;
each R⁷ is cyclopropyl; and R⁶ is fluoro;
each R⁷ is isopropyl; and R⁶ is methoxy;
each R⁷ is isopropyl; and R⁶ is trifluoromethoxy;
each R⁷ is chloro; and R⁶ is trifluoromethyl; or
each R⁷ is chloro; and R⁶ is trifluoromethoxy.

In some embodiments, of the compound of formula AA,

the substituted ring B is

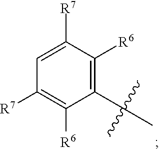

and R⁶ and R⁷ are one of the following combinations:
each R⁶ is independently C₁-C₆ alkyl, and each R⁷ is independently C₁-C₆ alkyl optionally substituted with one or more halo;
each R⁶ is independently C₁-C₆ alkyl and each R⁷ is independently C₁-C₆ alkyl;
each R⁶ is independently C₁-C₆ alkyl, and each R⁷ is independently C₁-C₆ alkyl substituted with one or more halo;
each R⁶ is independently C₁-C₆ alkyl, and each R⁷ is independently C₃-C₇ cycloalkyl;
each R⁶ is independently C₁-C₆ alkyl, and each R⁷ is independently halo;
each R⁶ is independently C₁-C₆ alkyl, and R⁷ is cyano;
each R⁶ is independently C₃-C₇ cycloalkyl, and each R⁷ is independently C₃-C₇ cycloalkyl;
each R⁶ is independently C₃-C₇ cycloalkyl, and each R⁷ is independently halo;
each R⁶ is independently cyclopropyl and each R⁷ is independently halo;
each R⁶ is independently C₁-C₆ alkyl, and each R⁷ is independently C₁-C₆ alkoxy optionally substituted with one or more halo;
each R⁶ is independently C₁-C₆ alkyl, and each R⁷ is independently C₁-C₆ alkoxy;
each R⁶ is independently C₁-C₆ alkyl, and each R⁷ is independently C₁-C₆ alkoxy substituted with one or more halo;
each R⁶ is independently halo, and each R⁷ is independently C₁-C₆ haloalkyl;
each R⁶ is independently halo, and each R⁷ is independently C₁-C₆ haloalkoxy;
each R⁶ is independently C₁-C₆ alkoxy; and each R⁷ is independently halo;
each R⁶ is independently C₁-C₆ alkoxy; and R⁷ is chloro;
each R⁷ is independently C₁-C₆ alkyl, and each R⁶ is independently C₁-C₆ alkyl optionally substituted with one or more halo;
each R⁷ is independently C₁-C₆ alkyl, and each R⁶ is independently C₁-C₆ alkyl substituted with one or more halo;
each R⁷ is independently C₁-C₆ alkyl, and each R⁶ is independently C₃-C₇ cycloalkyl;
each R⁷ is independently C₁-C₆ alkyl, and each R⁶ is independently halo;
each R⁷ is independently C₁-C₆ alkyl and each R⁶ is independently halo;
each R⁷ is independently C₁-C₆ alkyl, and R⁶ is cyano;
each R⁷ is independently C₃-C₇ cycloalkyl, and each R⁶ is independently C₃-C₇ cycloalkyl
each R⁷ is independently C₃-C₇ cycloalkyl, and each R⁶ is independently halo;
each R⁷ is independently C₃-C₇ cycloalkyl and each R⁶ is independently halo;
each R⁷ is independently C₁-C₆ alkyl, and each R⁶ is independently C₁-C₆ alkoxy optionally substituted with one or more halo;
each R⁷ is independently C₁-C₆ alkyl, and each R⁶ is independently C₁-C₆ alkoxy;
each R⁷ is independently C₁-C₆ alkyl, and each R⁶ is independently C₁-C₆ alkoxy substituted with one or more halo;
each R⁷ is independently halo, and each R⁶ is independently C₁-C₆ haloalkyl;
each R⁷ is independently halo, and each R⁶ is independently C₁-C₆ haloalkoxy;
each R⁷ is independently C₁-C₆ alkoxy; and each R⁶ is independently halo;
each R⁷ is independently C₁-C₆ alkoxy; and R⁶ is chloro;
two pairs, each of one R⁶ and one R⁷, are on adjacent atoms, and each pair of one R⁶ and one R⁷ taken together with the atoms connecting them form a C₄-C₈ aliphatic carbocyclic ring;
two pairs, each of one R⁶ and one R⁷, are on adjacent atoms, and each pair of one R⁶ and one R⁷ taken together with the atoms connecting them form a C₄-C₆ aliphatic carbocyclic ring optionally substituted with one or more hydroxy, oxo, or C₁-C₆ alkyl;
two pairs, each of one R⁶ and one R⁷, are on adjacent atoms, and one pair of one R⁶ and one R⁷ taken together with the atoms connecting them form a C₄-C₆ aliphatic carbocyclic ring optionally substituted with one or more hydroxy, oxo, or C₁-C₆ alkyl, and the other pair of one R⁶ and one R⁷ taken together with the atoms connecting them form a 5-to-6-membered heterocyclic ring containing 1 heteroatom independently selected from O, N, and S, wherein the heterocyclic ring is optionally substituted with one or more hydroxy, oxo, or C₁-C₆ alkyl; or
two pairs, each of one R⁶ and one R⁷, are on adjacent atoms, and each pair of one R⁶ and one R⁷ taken together with the atoms connecting them form a 5-to-6-membered heterocyclic ring containing 1 heteroatom independently selected from O, N, and S, wherein the heterocyclic ring optionally substituted with one or more hydroxy, oxo, or C₁-C₆ alkyl.

In some embodiments, of the compound of formula AA,

the substituted ring B is

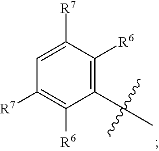

and R⁶ and R⁷ are one of the following combinations:
each R⁶ is isopropyl; and each R⁷ is methyl;
each R⁶ is isopropyl; and each R⁷ is isopropyl;
each R⁶ is isopropyl; and each R⁷ is trifluoromethyl;
each R⁶ is isopropyl; and each R⁷ is cyclopropyl;
each R⁶ is isopropyl; and each R⁷ is chloro;
each R⁶ is isopropyl; and each R⁷ is fluoro;
each R⁶ is ethyl; and each R⁷ is fluoro;
each R⁶ is isopropyl; and each R⁷ is cyano;
each R⁶ is cyclopropyl; and each R⁷ is cyclopropyl;
each R⁶ is cyclopropyl; and each R⁷ is chloro;
each R⁶ is cyclopropyl; and each R⁷ is fluoro;
each R⁶ is isopropyl; and each R⁷ is methoxy;
each R⁶ is isopropyl; and each R⁷ is trifluoromethoxy;
each R⁶ is chloro; and each R⁷ is trifluoromethyl;
each R⁶ is chloro; and each R⁷ is trifluoromethoxy;
each R⁷ is isopropyl; and each R⁶ is methyl;
each R⁷ is isopropyl; and each R⁶ is trifluoromethyl;
each R⁷ is isopropyl; and each R⁶ is cyclopropyl;
each R⁷ is isopropyl; and each R⁶ is chloro;
each R⁷ is ethyl; and each R⁶ is fluoro;
each R⁷ is isopropyl; and each R⁶ is cyano;
each R⁷ is cyclopropyl; and each R⁶ is cyclopropyl;
each R⁷ is cyclopropyl; and each R⁶ is chloro;
each R⁷ is cyclopropyl; and each R⁶ is fluoro;
each R⁷ is isopropyl; and each R⁶ is methoxy;
each R⁷ is isopropyl; and each R⁶ is trifluoromethoxy;
each R⁷ is chloro; and each R⁶ is trifluoromethyl;
each R⁷ is chloro; and each R⁶ is trifluoromethoxy;
one R⁶ is isopropyl; the other R⁶ is trifluoromethyl; and each R⁷ is chloro;
each R⁶ is isopropyl; one R⁷ is fluoro; and the other R⁷ is cyano;
two pairs, each of one R⁶ and one R⁷, are on adjacent atoms, and each pair of one R⁶ and one R⁷ taken together with the atoms connecting them form a C₅ aliphatic carbocyclic ring;
two pairs, each of one R⁶ and one R⁷, are on adjacent atoms, and each pair of one R⁶ and one R⁷ taken together with the atoms connecting them form a C₄ aliphatic carbocyclic ring optionally substituted with one or more hydroxy, oxo, or methyl;
two pairs, each of one R⁶ and one R⁷, are on adjacent atoms, and each pair of one R⁶ and one R⁷ taken together with the atoms connecting them form a C₅ aliphatic carbocyclic ring optionally substituted with one or more hydroxy, oxo, or methyl;
two pairs, each of one R⁶ and one R⁷, are on adjacent atoms, and each pair of one R⁶ and one R⁷ taken together with the atoms connecting them form a C₆ aliphatic carbocyclic ring optionally substituted with one or more hydroxy, oxo, or methyl;
two pairs, each of one R⁶ and one R⁷, are on adjacent atoms, and each pair of one R⁶ and one R⁷ taken together with the atoms connecting them form a 5-membered heterocyclic ring containing 1 heteroatom independently selected from O, N, and S, wherein the heterocyclic ring is optionally substituted with one or more hydroxy, oxo, or methyl;
two pairs, each of one R⁶ and one R⁷, are on adjacent atoms, and each pair of one R⁶ and one R⁷ taken together with the atoms connecting them form a 6-membered heterocyclic ring containing 1 heteroatom independently selected from O, N, and S, wherein the heterocyclic ring is optionally substituted with one or more hydroxy, oxo, or methyl; or
two pairs, each of one R⁶ and one R⁷, are on adjacent atoms, one pair of one R⁶ and one R⁷ taken together with the atoms connecting them form a 5-membered heterocyclic ring containing 1 heteroatom independently selected from O, N, and S, wherein the heterocyclic ring is optionally substituted with one or more hydroxy, oxo, or methyl, and the other pair of one R⁶ and one R⁷ taken together with the atoms connecting them form a C₅ aliphatic carbocyclic ring optionally substituted with one or more hydroxy, oxo, or methyl .

In some embodiments, of the compound of formula AA,

the substituted ring B is

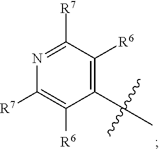

and R⁶ and R⁷ are one of the following combinations:
each R⁶ is independently C₁-C₆ alkyl, and each R⁷ is independently C₁-C₆ alkyl optionally substituted with one or more halo;
each R⁶ is independently C₁-C₆ alkyl and each R⁷ is independently C₁-C₆ alkyl;
each R⁶ is independently C₁-C₆ alkyl, and each R⁷ is independently C₁-C₆ alkyl substituted with one or more halo;
each R⁶ is independently C₁-C₆ alkyl, and each R⁷ is independently C₃-C₇ cycloalkyl;
each R⁶ is independently C₁-C₆ alkyl, and each R⁷ is independently halo;
each R⁶ is independently C₁-C₆ alkyl, and R⁷ is cyano;
each R⁶ is independently C₃-C₇ cycloalkyl, and each R⁷ is independently C₃-C₇ cycloalkyl;
each R⁶ is independently C₃-C₇ cycloalkyl, and each R⁷ is independently halo;
each R⁶ is independently cyclopropyl and each R⁷ is independently halo;
each R⁶ is independently C₁-C₆ alkyl, and each R⁷ is independently C₁-C₆ alkoxy optionally substituted with one or more halo;
each R⁶ is independently C₁-C₆ alkyl, and each R⁷ is independently C₁-C₆ alkoxy;
each R⁶ is independently C₁-C₆ alkyl, and each R⁷ is independently C₁-C₆ alkoxy substituted with one or more halo;
each R⁶ is independently halo, and each R⁷ is independently C₁-C₆ haloalkyl;
each R⁶ is independently halo, and each R⁷ is independently C₁-C₆ haloalkoxy;
each R⁶ is independently C₁-C₆ alkoxy; and each R⁷ is independently halo;
each R⁶ is independently C₁-C₆ alkoxy; and R⁷ is chloro;
each R⁷ is independently C₁-C₆ alkyl, and each R⁶ is independently C₁-C₆ alkyl optionally substituted with one or more halo;
each R⁷ is independently C₁-C₆ alkyl, and each R⁶ is independently C₁-C₆ alkyl substituted with one or more halo;
each R⁷ is independently C₁-C₆ alkyl, and each R⁶ is independently C₃-C₇ cycloalkyl;
each R⁷ is independently C₁-C₆ alkyl, and each R⁶ is independently halo;
each R⁷ is independently C₁-C₆ alkyl and each R⁶ is independently halo;
each R⁷ is independently C₁-C₆ alkyl, and R⁶ is cyano;
each R⁷ is independently C₃-C₇ cycloalkyl, and each R⁶ is independently C₃-C₇ cycloalkyl;
each R⁷ is independently C₃-C₇ cycloalkyl, and each R⁶ is independently halo;
each R⁷ is independently C₃-C₇ cycloalkyl and each R⁶ is independently halo;
each R⁷ is independently C₁-C₆ alkyl, and each R⁶ is independently C₁-C₆ alkoxy optionally substituted with one or more halo;
each R⁷ is independently C₁-C₆ alkyl, and each R⁶ is independently C₁-C₆ alkoxy;
each R⁷ is independently C₁-C₆ alkyl, and each R⁶ is independently C₁-C₆ alkoxy substituted with one or more halo;
each R⁷ is independently halo, and each R⁶ is independently C₁-C₆ haloalkyl;
each R⁷ is independently halo, and each R⁶ is independently C₁-C₆ haloalkoxy;
each R⁷ is independently C₁-C₆ alkoxy; and each R⁶ is independently halo;
each R⁷ is independently C₁-C₆ alkoxy; and R⁶ is chloro;
two pairs, each of one R⁶ and one R⁷, are on adjacent atoms, and each pair of one R⁶ and one R⁷ taken together with the atoms connecting them form a C₄-C₈ aliphatic carbocyclic ring;
two pairs, each of one R⁶ and one R⁷ on adjacent atoms taken together with the atoms connecting them form a C₄-C₆ aliphatic carbocyclic ring optionally substituted with one or more hydroxy, oxo, or C₁-C₆ alkyl; or
two pairs, each of one R⁶ and one R⁷ on adjacent atoms taken together with the atoms connecting them form a 5-to-6-membered heterocyclic ring containing 1 heteroatom independently selected from O, N, and S, wherein the heterocyclic ring optionally substituted with one or more hydroxy, oxo, or C₁-C₆ alkyl.

In some embodiments, of the compound of formula AA,

the substituted ring B is

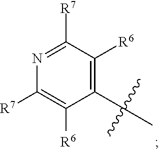

and R⁶ and Ware one of the following combinations:
each R⁶ is isopropyl; and each R⁷ is methyl;
each R⁶ is isopropyl; and each R⁷ is isopropyl;
each R⁶ is isopropyl; and each R⁷ is trifluoromethyl;
each R⁶ is isopropyl; and each R⁷ is cyclopropyl;
each R⁶ is isopropyl; and each R⁷ is chloro;
each R⁶ is isopropyl; and each R⁷ is fluoro;
each R⁶ is ethyl; and each R⁷ is fluoro;
each R⁶ is isopropyl; and each R⁷ is cyano;
each R⁶ is cyclopropyl; and each R⁷ is cyclopropyl;
each R⁶ is cyclopropyl; and each R⁷ is chloro;
each R⁶ is cyclopropyl; and each R⁷ is fluoro;
each R⁶ is isopropyl; and each R⁷ is methoxy;
each R⁶ is isopropyl; and each R⁷ is trifluoromethoxy;
each R⁶ is chloro; and each R⁷ is trifluoromethyl;
each R⁶ is chloro; and each R⁷ is trifluoromethoxy;
each R⁷ is isopropyl; and each R⁶ is methyl;
each R⁷ is isopropyl; and each R⁶ is trifluoromethyl;
each R⁷ is isopropyl; and each R⁶ is cyclopropyl;
each R⁷ is isopropyl; and each R⁶ is chloro;
each R⁷ is ethyl; and each R⁶ is fluoro;
each R⁷ is isopropyl; and each R⁶ is cyano;
each R⁷ is cyclopropyl; and each R⁶ is cyclopropyl;
each R⁷ is cyclopropyl; and each R⁶ is chloro;
each R⁷ is cyclopropyl; and each R⁶ is fluoro;
each R⁷ is isopropyl; and each R⁶ is methoxy;
each R⁷ is isopropyl; and each R⁶ is trifluoromethoxy;
each R⁷ is chloro; and each R⁶ is trifluoromethyl;
each R⁷ is chloro; and each R⁶ is trifluoromethoxy;
one R⁶ is isopropyl; the other R⁶ is trifluoromethyl; and each R⁷ is chloro;
each R⁶ is isopropyl; one R⁷ is fluoro; and the other R⁷ is cyano; or
two pairs, each of one R⁶ and one R⁷ on adjacent atoms taken together with the atoms connecting them form a C₄ aliphatic carbocyclic ring optionally substituted with one or more hydroxy, oxo, or methyl;
two pairs, each of one R⁶ and one R⁷ on adjacent atoms taken together with the atoms connecting them form a C₅ aliphatic carbocyclic ring optionally substituted with one or more hydroxy, oxo, or methyl;
two pairs, each of one R⁶ and one R⁷ on adjacent atoms taken together with the atoms connecting them form a C₆ aliphatic carbocyclic ring optionally substituted with one or more hydroxy, oxo, or methyl;
two pairs, each of one R⁶ and one R⁷ on adjacent atoms taken together with the atoms connecting them form a 5-membered heterocyclic ring containing 1 heteroatom independently selected from O, N, and S, wherein the heterocyclic ring is optionally substituted with one or more hydroxy, oxo, or methyl;
two pairs, each of one R⁶ and one R⁷ on adjacent atoms taken together with the atoms connecting them form a 6-membered heterocyclic ring containing 1 heteroatom independently selected from O, N, and S, wherein the heterocyclic ring is optionally substituted with one or more hydroxy, oxo, or methyl; or
two pairs, each of one R⁶ and one R⁷, are on adjacent atoms, and each pair of one R⁶ and one R⁷ taken together with the atoms connecting them form a C₅ aliphatic carbocyclic ring. 1

In some embodiments, of the compound of formula AA,

the substituted ring B is

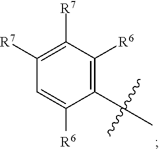

and R⁶ and R⁷ are one of the following combinations:
each R⁶ is independently C₁-C₆ alkyl, and each R⁷ is independently C₁-C₆ alkyl optionally substituted with one or more halo;
each R⁶ is independently C₁-C₆ alkyl and each R⁷ is independently C₁-C₆ alkyl;
each R⁶ is independently C₁-C₆ alkyl, and each R⁷ is independently C₁-C₆ alkyl substituted with one or more halo;
each R⁶ is independently C₁-C₆ alkyl, and each R⁷ is independently C₃-C₇ cycloalkyl;
each R⁶ is independently C₁-C₆ alkyl, and each R⁷ is independently halo;
each R⁶ is independently C₁-C₆ alkyl, and R⁷ is cyano;
each R⁶ is independently C₃-C₇ cycloalkyl, and each R⁷ is independently C₃-C₇ cycloalkyl;
each R⁶ is independently C₃-C₇ cycloalkyl, and each R⁷ is independently halo;
each R⁶ is independently cyclopropyl and each R⁷ is independently halo;
each R⁶ is independently C₁-C₆ alkyl, and each R⁷ is independently C₁-C₆ alkoxy optionally substituted with one or more halo;
each R⁶ is independently C₁-C₆ alkyl, and each R⁷ is independently C₁-C₆ alkoxy;
each R⁶ is independently C₁-C₆ alkyl, and each R⁷ is independently C₁-C₆ alkoxy substituted with one or more halo;
each R⁶ is independently halo, and each R⁷ is independently C₁-C₆ haloalkyl;
each R⁶ is independently halo, and each R⁷ is independently C₁-C₆ haloalkoxy;
each R⁶ is independently C₁-C₆ alkoxy; and each R⁷ is independently halo;
each R⁶ is independently C₁-C₆ alkoxy; and R⁷ is chloro;
each R⁷ is independently C₁-C₆ alkyl, and each R⁶ is independently C₁-C₆ alkyl optionally substituted with one or more halo;
each R⁷ is independently C₁-C₆ alkyl, and each R⁶ is independently C₁-C₆ alkyl substituted with one or more halo;
each R⁷ is independently C₁-C₆ alkyl, and each R⁶ is independently C₃-C₇ cycloalkyl;
each R⁷ is independently C₁-C₆ alkyl, and each R⁶ is independently halo;
each R⁷ is independently C₁-C₆ alkyl and each R⁶ is independently halo;
each R⁷ is independently C₁-C₆ alkyl, and R⁶ is cyano;
each R⁷ is independently C₃-C₇ cycloalkyl, and each R⁶ is independently C₃-C₇ cycloalkyl;
each R⁷ is independently C₃-C₇ cycloalkyl, and each R⁶ is independently halo;
each R⁷ is independently C₃-C₇ cycloalkyl and each R⁶ is independently halo;
each R⁷ is independently C₁-C₆ alkyl, and each R⁶ is independently C₁-C₆ alkoxy optionally substituted with one or more halo;
each R⁷ is independently C₁-C₆ alkyl, and each R⁶ is independently C₁-C₆ alkoxy;
each R⁷ is independently C₁-C₆ alkyl, and each R⁶ is independently C₁-C₆ alkoxy substituted with one or more halo;
each R⁷ is independently halo, and each R⁶ is independently C₁-C₆ haloalkyl;
each R⁷ is independently halo, and each R⁶ is independently C₁-C₆ haloalkoxy;
each R⁷ is independently C₁-C₆ alkoxy; and each R⁶ is independently halo; or
each R⁷ is independently C₁-C₆ alkoxy; and R⁶ is chloro.

In some embodiments of the compound of formula AA,

the substituted ring B is

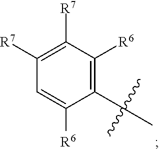

and R⁶ and R⁷ are one of the following combinations:
each R⁶ is isopropyl; and each R⁷ is methyl;
each R⁶ is isopropyl; and each R⁷ is isopropyl;
each R⁶ is isopropyl; and each R⁷ is trifluoromethyl;
each R⁶ is isopropyl; and each R⁷ is cyclopropyl;
each R⁶ is isopropyl; and each R⁷ is chloro;
each R⁶ is isopropyl; and each R⁷ is fluoro;
each R⁶ is ethyl; and each R⁷ is fluoro;
each R⁶ is isopropyl; and each R⁷ is cyano;
each R⁶ is cyclopropyl; and each R⁷ is cyclopropyl;
each R⁶ is cyclopropyl; and each R⁷ is chloro;
each R⁶ is cyclopropyl; and each R⁷ is fluoro;
each R⁶ is isopropyl; and each R⁷ is methoxy;
each R⁶ is isopropyl; and each R⁷ is trifluoromethoxy;
each R⁶ is chloro; and each R⁷ is trifluoromethyl;
each R⁶ is chloro; and each R⁷ is trifluoromethoxy;
each R⁷ is isopropyl; and each R⁶ is methyl;
each R⁷ is isopropyl; and each R⁶ is trifluoromethyl;
each R⁷ is isopropyl; and each R⁶ is cyclopropyl;
each R⁷ is isopropyl; and each R⁶ is chloro;
each R⁷ is ethyl; and each R⁶ is fluoro;
each R⁷ is isopropyl; and each R⁶ is cyano;
each R⁷ is cyclopropyl; and each R⁶ is cyclopropyl;
each R⁷ is cyclopropyl; and each R⁶ is chloro;
each R⁷ is cyclopropyl; and each R⁶ is fluoro;
each R⁷ is isopropyl; and each R⁶ is methoxy;
each R⁷ is isopropyl; and each R⁶ is trifluoromethoxy;
each R⁷ is chloro; and each R⁶ is trifluoromethyl;
each R⁷ is chloro; and each R⁶ is trifluoromethoxy;
one R⁶ is isopropyl; the other R⁶ is trifluoromethyl; and R⁷ is chloro; or
R⁶ is isopropyl; one R⁷ is fluoro; and the other R⁷ is cyano.

In some embodiments, of the compound of formula AA,

the substituted ring B is

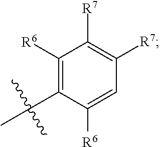

and R⁶ and R⁷ are one of the following combinations:
each R⁶ is independently C₁-C₆ alkyl, and each R⁷ is independently C₁-C₆ alkyl optionally substituted with one or more halo;
each R⁶ is independently C₁-C₆ alkyl and each R⁷ is independently C₁-C₆ alkyl;
each R⁶ is independently C₁-C₆ alkyl, and each R⁷ is independently C₁-C₆ alkyl substituted with one or more halo;
each R⁶ is independently C₁-C₆ alkyl, and each R⁷ is independently C₃-C₇ cycloalkyl;
each R⁶ is independently C₁-C₆ alkyl, and each R⁷ is independently halo;
each R⁶ is independently C₁-C₆ alkyl, and each R⁷ is cyano;
each R⁶ is independently C₃-C₇ cycloalkyl, and each R⁷ is independently C₃-C₇ cycloalkyl;
each R⁶ is independently C₃-C₇ cycloalkyl, and each R⁷ is independently halo;
each R⁶ is independently cyclopropyl and each R⁷ is independently halo;
each R⁶ is independently C₁-C₆ alkyl, and each R⁷ is independently C₁-C₆ alkoxy optionally substituted with one or more halo;
each R⁶ is independently C₁-C₆ alkyl, and each R⁷ is independently C₁-C₆ alkoxy;
each R⁶ is independently C₁-C₆ alkyl, and each R⁷ is independently C₁-C₆ alkoxy substituted with one or more halo;
each R⁶ is independently halo, and each R⁷ is independently C₁-C₆ haloalkyl;
each R⁶ is independently halo, and each R⁷ is independently C₁-C₆ haloalkoxy;
each R⁶ is independently C₁-C₆ alkoxy; and each R⁷ is independently halo;
each R⁶ is independently C₁-C₆ alkoxy; and each R⁷ is chloro;
each R⁷ is independently C₁-C₆ alkyl, and each R⁶ is independently C₁-C₆ alkyl optionally substituted with one or more halo;
each R⁷ is independently C₁-C₆ alkyl, and each R⁶ is independently C₁-C₆ alkyl substituted with one or more halo;
each R⁷ is independently C₁-C₆ alkyl, and each R⁶ is independently C₃-C₇ cycloalkyl;
each R⁷ is independently C₁-C₆ alkyl, and each R⁶ is independently halo;
each R⁷ is independently C₁-C₆ alkyl and each R⁶ is independently halo;
each R⁷ is independently C₁-C₆ alkyl, and each R⁶ is cyano;
each R⁷ is independently C₃-C₇ cycloalkyl, and each R⁶ is independently C₃-C₇ cycloalkyl;
each R⁷ is independently C₃-C₇ cycloalkyl, and each R⁶ is independently halo;
each R⁷ is independently C₃-C₇ cycloalkyl and each R⁶ is independently halo;
each R⁷ is independently C₁-C₆ alkyl, and each R⁶ is independently C₁-C₆ alkoxy optionally substituted with one or more halo;
each R⁷ is independently C₁-C₆ alkyl, and each R⁶ is independently C₁-C₆ alkoxy;
each R⁷ is independently C₁-C₆ alkyl, and each R⁶ is independently C₁-C₆ alkoxy substituted with one or more halo;
each R⁷ is independently halo, and each R⁶ is independently C₁-C₆ haloalkyl;
each R⁷ is independently halo, and each R⁶ is independently C₁-C₆ haloalkoxy;
each R⁷ is independently C₁-C₆ alkoxy; and each R⁶ is independently halo;
each R⁷ is independently C₁-C₆ alkoxy; and each R⁶ is chloro;
R⁶ and R⁷ on adjacent atoms taken together with the atoms connecting them form a C₄-C₆ aliphatic carbocyclic ring optionally substituted with one or more hydroxy, oxo, or C₁-C₆ alkyl; and one R⁶ is halo or cyano; or
R⁶ and R⁷ on adjacent atoms taken together with the atoms connecting them form a 5-to-6-membered heterocyclic ring containing 1 heteroatom independently selected from O, N, and S, wherein the heterocyclic ring optionally substituted with one or more hydroxy, oxo, or C₁-C₆ alkyl; and one R⁶ is halo or cyano.

In some embodiments, of the compound of formula AA,

the substituted ring B is

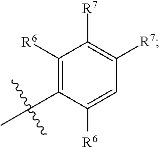

and R⁶ and R⁷ are one of the following combinations:
each R⁶ is isopropyl; and each R⁷ is methyl;
each R⁶ is isopropyl; and each R⁷ is isopropyl;
each R⁶ is isopropyl; and each R⁷ is trifluoromethyl;
each R⁶ is isopropyl; and each R⁷ is cyclopropyl;
each R⁶ is isopropyl; and each R⁷ is chloro;
each R⁶ is isopropyl; and each R⁷ is fluoro;
each R⁶ is ethyl; and each R⁷ is fluoro;
each R⁶ is isopropyl; and each R⁷ is cyano;
each R⁶ is cyclopropyl; and each R⁷ is cyclopropyl;
each R⁶ is cyclopropyl; and each R⁷ is chloro;
each R⁶ is cyclopropyl; and each R⁷ is fluoro;
each R⁶ is isopropyl; and each R⁷ is methoxy;
each R⁶ is isopropyl; and each R⁷ is trifluoromethoxy;
each R⁶ is chloro; and each R⁷ is trifluoromethyl;
each R⁶ is chloro; and each R⁷ is trifluoromethoxy;
each R⁷ is isopropyl; and each R⁶ is methyl;
each R⁷ is isopropyl; and each R⁶ is trifluoromethyl;
each R⁷ is isopropyl; and each R⁶ is cyclopropyl;
each R⁷ is isopropyl; and each R⁶ is chloro;
each R⁷ is ethyl; and each R⁶ is fluoro;
each R⁷ is isopropyl; and each R⁶ is cyano;
each R⁷ is cyclopropyl; and each R⁶ is cyclopropyl;
each R⁷ is cyclopropyl; and each R⁶ is chloro;
each R⁷ is cyclopropyl; and each R⁶ is fluoro;
each R⁷ is isopropyl; and each R⁶ is methoxy;
each R⁷ is isopropyl; and each R⁶ is trifluoromethoxy;
each R⁷ is chloro; and each R⁶ is trifluoromethyl;
each R⁷ is chloro; and each R⁶ is trifluoromethoxy;
one R⁶ is isopropyl; the other R⁶ is trifluoromethyl; and each R⁷ is chloro;
each R⁶ is isopropyl; one R⁷ is fluoro; and the other R⁷ is cyano;
R⁶ and R⁷ on adjacent atoms taken together with the atoms connecting them form a C₄ aliphatic carbocyclic ring; and one R⁶ is chloro, fluoro, or cyano;
R⁶ and R⁷ on adjacent atoms taken together with the atoms connecting them form a C₅ aliphatic carbocyclic ring; and one R⁶ is chloro, fluoro, or cyano;
R⁶ and R⁷ on adjacent atoms taken together with the atoms connecting them form a C₆ aliphatic carbocyclic ring; and one R⁶ is chloro, fluoro, or cyano;
R⁶ and R⁷ on adjacent atoms taken together with the atoms connecting them form a 5-membered heterocyclic ring containing 1 heteroatoms independently selected from O, N, and S; and one R⁶ is chloro, fluoro, or cyano;
R⁶ and R⁷ on adjacent atoms taken together with the atoms connecting them form a 6-membered heterocyclic ring containing 1 heteroatoms independently selected from O, N, and S; and one R⁶ is chloro, fluoro, or cyano; or
R⁶ and R⁷ on adjacent atoms taken together with the atoms connecting them form a C₅ aliphatic carbocyclic ring; and one R⁶ is chloro, fluoro, or cyano.

In some embodiments, of the compound of formula AA,

the substituted ring B is

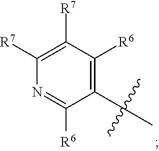

and R⁶ and R⁷ are one of the following combinations:
each R⁶ is independently C₁-C₆ alkyl, and each R⁷ is independently C₁-C₆ alkyl optionally substituted with one or more halo;
each R⁶ is independently C₁-C₆ alkyl and each R⁷ is independently C₁-C₆ alkyl;
each R⁶ is independently C₁-C₆ alkyl, and each R⁷ is independently C₁-C₆ alkyl substituted with one or more halo;
each R⁶ is independently C₁-C₆ alkyl, and each R⁷ is independently C₃-C₇ cycloalkyl;
each R⁶ is independently C₁-C₆ alkyl, and each R⁷ is independently halo;
each R⁶ is independently C₁-C₆ alkyl, and R⁷ is cyano;
each R⁶ is independently C₃-C₇ cycloalkyl, and each R⁷ is independently C₃-C₇ cycloalkyl;
each R⁶ is independently C₃-C₇ cycloalkyl, and each R⁷ is independently halo;
each R⁶ is independently cyclopropyl and each R⁷ is independently halo;
each R⁶ is independently C₁-C₆ alkyl, and each R⁷ is independently C₁-C₆ alkoxy optionally substituted with one or more halo;
each R⁶ is independently C₁-C₆ alkyl, and each R⁷ is independently C₁-C₆ alkoxy;
each R⁶ is independently C₁-C₆ alkyl, and each R⁷ is independently C₁-C₆ alkoxy substituted with one or more halo;
each R⁶ is independently halo, and each R⁷ is independently C₁-C₆ haloalkyl;
each R⁶ is independently halo, and each R⁷ is independently C₁-C₆ haloalkoxy;
each R⁶ is independently C₁-C₆ alkoxy; and each R⁷ is independently halo;
each R⁶ is independently C₁-C₆ alkoxy; and R⁷ is chloro;
each R⁷ is independently C₁-C₆ alkyl, and each R⁶ is independently C₁-C₆ alkyl optionally substituted with one or more halo;
each R⁷ is independently C₁-C₆ alkyl, and each R⁶ is independently C₁-C₆ alkyl substituted with one or more halo;
each R⁷ is independently C₁-C₆ alkyl, and each R⁶ is independently C₃-C₇ cycloalkyl;
each R⁷ is independently C₁-C₆ alkyl, and each R⁶ is independently halo;
each R⁷ is independently C₁-C₆ alkyl and each R⁶ is independently halo;
each R⁷ is independently C₁-C₆ alkyl, and R⁶ is cyano;
each R⁷ is independently C₃-C₇ cycloalkyl, and each R⁶ is independently C₃-C₇ cycloalkyl;
each R⁷ is independently C₃-C₇ cycloalkyl, and each R⁶ is independently halo;
each R⁷ is independently C₃-C₇ cycloalkyl and each R⁶ is independently halo;
each R⁷ is independently C₁-C₆ alkyl, and each R⁶ is independently C₁-C₆ alkoxy optionally substituted with one or more halo;
each R⁷ is independently C₁-C₆ alkyl, and each R⁶ is independently C₁-C₆ alkoxy;
each R⁷ is independently C₁-C₆ alkyl, and each R⁶ is independently C₁-C₆ alkoxy substituted with one or more halo;
each R⁷ is independently halo, and each R⁶ is independently C₁-C₆ haloalkyl;
each R⁷ is independently halo, and each R⁶ is independently C₁-C₆ haloalkoxy;
each R⁷ is independently C₁-C₆ alkoxy; and each R⁶ is independently halo; or
each R⁷ is independently C₁-C₆ alkoxy; and R⁶ is chloro.

In some embodiments, of the compound of formula AA,

the substituted ring B is

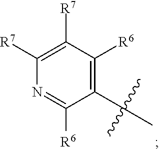

and R⁶ and R⁷ are one of the following combinations:
each R⁶ is isopropyl; and each R⁷ is methyl;
each R⁶ is isopropyl; and each R⁷ is isopropyl;
each R⁶ is isopropyl; and each R⁷ is trifluoromethyl;
each R⁶ is isopropyl; and each R⁷ is cyclopropyl;
each R⁶ is isopropyl; and each R⁷ is chloro;
each R⁶ is isopropyl; and each R⁷ is fluoro;
each R⁶ is ethyl; and each R⁷ is fluoro;
each R⁶ is isopropyl; and each R⁷ is cyano;
each R⁶ is cyclopropyl; and each R⁷ is cyclopropyl;
each R⁶ is cyclopropyl; and each R⁷ is chloro;
each R⁶ is cyclopropyl; and each R⁷ is fluoro;
each R⁶ is isopropyl; and each R⁷ is methoxy;
each R⁶ is isopropyl; and each R⁷ is trifluoromethoxy;
each R⁶ is chloro; and each R⁷ is trifluoromethyl;
each R⁶ is chloro; and each R⁷ is trifluoromethoxy;
each R⁷ is isopropyl; and each R⁶ is methyl;
each R⁷ is isopropyl; and each R⁶ is trifluoromethyl;
each R⁷ is isopropyl; and each R⁶ is cyclopropyl;
each R⁷ is isopropyl; and each R⁶ is chloro;
each R⁷ is ethyl; and each R⁶ is fluoro;
each R⁷ is isopropyl; and each R⁶ is cyano;
each R⁷ is cyclopropyl; and each R⁶ is cyclopropyl;
each R⁷ is cyclopropyl; and each R⁶ is chloro;
each R⁷ is cyclopropyl; and each R⁶ is fluoro;
each R⁷ is isopropyl; and each R⁶ is methoxy;
each R⁷ is isopropyl; and each R⁶ is trifluoromethoxy;
each R⁷ is chloro; and each R⁶ is trifluoromethyl;
each R⁷ is chloro; and each R⁶ is trifluoromethoxy;
one R⁶ is isopropyl; the other R⁶ is trifluoromethyl; and each R⁷ is chloro; or
each R⁶ is isopropyl; one R⁷ is fluoro; and the other R⁷ is cyano.

In some embodiments, of the compound of formula AA,

the substituted ring B is

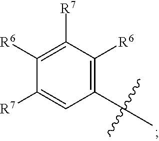

and R⁶ and R⁷ are one of the following combinations:
each R⁶ is independently C₁-C₆ alkyl, and each R⁷ is independently C₁-C₆ alkyl optionally substituted with one or more halo;
each R⁶ is independently C₁-C₆ alkyl and each R⁷ is independently C₁-C₆ alkyl;
each R⁶ is independently C₁-C₆ alkyl, and each R⁷ is independently C₁-C₆ alkyl substituted with one or more halo;
each R⁶ is independently C₁-C₆ alkyl, and each R⁷ is independently C₃-C₇ cycloalkyl;
each R⁶ is independently C₁-C₆ alkyl, and each R⁷ is independently halo;
each R⁶ is independently C₁-C₆ alkyl, and R⁷ is cyano;
each R⁶ is independently C₃-C₇ cycloalkyl, and each R⁷ is independently C₃-C₇ cycloalkyl;
each R⁶ is independently C₃-C₇ cycloalkyl, and each R⁷ is independently halo;
each R⁶ is independently cyclopropyl and each R⁷ is independently halo;
each R⁶ is independently C₁-C₆ alkyl, and each R⁷ is independently C₁-C₆ alkoxy optionally substituted with one or more halo;
each R⁶ is independently C₁-C₆ alkyl, and each R⁷ is independently C₁-C₆ alkoxy;
each R⁶ is independently C₁-C₆ alkyl, and each R⁷ is independently C₁-C₆ alkoxy substituted with one or more halo;
each R⁶ is independently halo, and each R⁷ is independently C₁-C₆ haloalkyl;
each R⁶ is independently halo, and each R⁷ is independently C₁-C₆ haloalkoxy;
each R⁶ is independently C₁-C₆ alkoxy; and each R⁷ is independently halo;
each R⁶ is independently C₁-C₆ alkoxy; and R⁷ is chloro;
each R⁷ is independently C₁-C₆ alkyl, and each R⁶ is independently C₁-C₆ alkyl optionally substituted with one or more halo;
each R⁷ is independently C₁-C₆ alkyl, and each R⁶ is independently C₁-C₆ alkyl substituted with one or more halo;
each R⁷ is independently C₁-C₆ alkyl, and each R⁶ is independently C₃-C₇ cycloalkyl;
each R⁷ is independently C₁-C₆ alkyl, and each R⁶ is independently halo;
each R⁷ is independently C₁-C₆ alkyl and each R⁶ is independently halo;
each R⁷ is independently C₁-C₆ alkyl, and R⁶ is cyano;
each R⁷ is independently C₃-C₇ cycloalkyl, and each R⁶ is independently C₃-C₇ cycloalkyl;
each R⁷ is independently C₃-C₇ cycloalkyl, and each R⁶ is independently halo;
each R⁷ is independently C₃-C₇ cycloalkyl and each R⁶ is independently halo;
each R⁷ is independently C₁-C₆ alkyl, and each R⁶ is independently C₁-C₆ alkoxy optionally substituted with one or more halo;
each R⁷ is independently C₁-C₆ alkyl, and each R⁶ is independently C₁-C₆ alkoxy;
each R⁷ is independently C₁-C₆ alkyl, and each R⁶ is independently C₁-C₆ alkoxy substituted with one or more halo;
each R⁷ is independently halo, and each R⁶ is independently C₁-C₆ haloalkyl;
each R⁷ is independently halo, and each R⁶ is independently C₁-C₆ haloalkoxy;
each R⁷ is independently C₁-C₆ alkoxy; and each R⁶ is independently halo;
each R⁷ is independently C₁-C₆ alkoxy; and R⁶ is chloro;
two pairs, each of one R⁶ and one R⁷, are on adjacent atoms, and each pair of one R⁶ and one R⁷ taken together with the atoms connecting them form a C₄-C₆ aliphatic carbocyclic ring optionally substituted with one or more hydroxy, oxo, or C₁-C₆ alkyl;
two pairs, each of one R⁶ and one R⁷, are on adjacent atoms, and each pair of one R⁶ and one
R⁷ taken together with the atoms connecting them form a 5-to-6-membered heterocyclic ring containing 1 heteroatom independently selected from O, N, and S, wherein the heterocyclic ring optionally substituted with one or more hydroxy, oxo, or C₁-C₆ alkyl; or
two pairs, each of one R⁶ and one R⁷, are on adjacent atoms, one pair of one R⁶ and one R⁷ taken together with the atoms connecting them form a C₄-C₆ aliphatic carbocyclic ring optionally substituted with one or more hydroxy, oxo, or C₁-C₆ alkyl, and the other pair of one R⁶ and one R⁷ taken together with the atoms connecting them form a 5-to-6-membered heterocyclic ring containing 1 heteroatom independently selected from O, N, and S, wherein the heterocyclic ring optionally substituted with one or more hydroxy, oxo, or C₁-C₆ alkyl.

In some embodiments, of the compound of formula AA,

the substituted ring B is

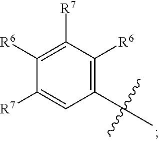

and R⁶ and R⁷ are one of the following combinations:
each R⁶ is isopropyl; and each R⁷ is methyl;
each R⁶ is isopropyl; and each R⁷ is isopropyl;
each R⁶ is isopropyl; and each R⁷ is trifluoromethyl;
each R⁶ is isopropyl; and each R⁷ is cyclopropyl;
each R⁶ is isopropyl; and each R⁷ is chloro;
each R⁶ is isopropyl; and each R⁷ is fluoro;
each R⁶ is ethyl; and each R⁷ is fluoro;
each R⁶ is isopropyl; and each R⁷ is cyano;
each R⁶ is cyclopropyl; and each R⁷ is cyclopropyl;
each R⁶ is cyclopropyl; and each R⁷ is chloro;
each R⁶ is cyclopropyl; and each R⁷ is fluoro;
each R⁶ is isopropyl; and each R⁷ is methoxy;
each R⁶ is isopropyl; and each R⁷ is trifluoromethoxy;
each R⁶ is chloro; and each R⁷ is trifluoromethyl;
each R⁶ is chloro; and each R⁷ is trifluoromethoxy;
each R⁷ is isopropyl; and each R⁶ is methyl;
each R⁷ is isopropyl; and each R⁶ is trifluoromethyl;
each R⁷ is isopropyl; and each R⁶ is cyclopropyl;
each R⁷ is isopropyl; and each R⁶ is chloro;
each R⁷ is ethyl; and each R⁶ is fluoro;
each R⁷ is isopropyl; and each R⁶ is cyano;
each R⁷ is cyclopropyl; and each R⁶ is cyclopropyl;
each R⁷ is cyclopropyl; and each R⁶ is chloro;
each R⁷ is cyclopropyl; and each R⁶ is fluoro;
each R⁷ is isopropyl; and each R⁶ is methoxy;
each R⁷ is isopropyl; and each R⁶ is trifluoromethoxy;
R⁷ is chloro; and each R⁶ is trifluoromethyl;
R⁷ is chloro; and each R⁶ is trifluoromethoxy;
one R⁶ is isopropyl; the other R⁶ is trifluoromethyl; and R⁷ is chloro;
R⁶ is isopropyl; one R⁷ is fluoro; and the other R⁷ is cyano; two pairs, each of one R⁶ and one R⁷, are on adjacent atoms, and each pair of one R⁶ and one R⁷ taken together with the atoms connecting them form a C₅ aliphatic carbocyclic ring;
two pairs, each of one R⁶ and one R⁷, are on adjacent atoms, and each pair of one R⁶ and one R⁷ taken together with the atoms connecting them form a C₄ aliphatic carbocyclic ring optionally substituted with one or more hydroxy, oxo, or methyl;
two pairs, each of one R⁶ and one R⁷, are on adjacent atoms, and each pair of one R⁶ and one R⁷ taken together with the atoms connecting them form a C₅ aliphatic carbocyclic ring optionally substituted with one or more hydroxy, oxo, or methyl;
two pairs, each of one R⁶ and one R⁷, are on adjacent atoms, and each pair of one R⁶ and one R⁷ taken together with the atoms connecting them form a C₆ aliphatic carbocyclic ring optionally substituted with one or more hydroxy, oxo, or methyl;
two pairs, each of one R⁶ and one R⁷, are on adjacent atoms, and each pair of one R⁶ and one R⁷ taken together with the atoms connecting them form a 5-membered heterocyclic ring containing 1 heteroatom independently selected from O, N, and S, wherein the heterocyclic ring is optionally substituted with one or more hydroxy, oxo, or methyl two pairs, each of one R⁶ and one R⁷, are on adjacent atoms, and each pair of one R⁶ and one R⁷ taken together with the atoms connecting them form a 6-membered heterocyclic ring containing 1 heteroatom independently selected from O, N, and S, wherein the heterocyclic ring is optionally substituted with one or more hydroxy, oxo, or methyl; or
two pairs, each of one R⁶ and one R⁷, are on adjacent atoms, one pair of one R⁶ and one R⁷ taken together with the atoms connecting them form a 5-membered heterocyclic ring containing 1 heteroatom independently selected from O, N, and S, wherein the heterocyclic ring is optionally substituted with one or more hydroxy, oxo, or methyl, and the other pair of one R⁶ and one R⁷ taken together with the atoms connecting them form a C₅ aliphatic carbocyclic ring optionally substituted with one or more hydroxy, oxo, or methyl .

In some embodiments, of the compound of formula AA,

the substituted ring B is

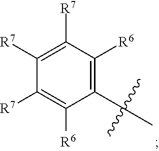

and R⁶ and R⁷ are one of the following combinations:
each R⁶ is independently C₁-C₆ alkyl, and R⁷ is C₁-C₆ alkyl optionally substituted with one or more halo;
each R⁶ is independently C₁-C₆ alkyl and R⁷ is C₁-C₆ alkyl;
each R⁶ is independently C₁-C₆ alkyl, and R⁷ is C₁-C₆ alkyl substituted with one or more halo;
each R⁶ is independently C₁-C₆ alkyl, and R⁷ is C₃-C₇ cycloalkyl;
each R⁶ is independently C₁-C₆ alkyl, and R⁷ is halo;
each R⁶ is independently C₁-C₆ alkyl, and R⁷ is cyano;
each R⁶ is independently C₃-C₇ cycloalkyl, and R⁷ is C₃-C₇ cycloalkyl;
each R⁶ is independently C₃-C₇ cycloalkyl, and R⁷ is halo;
each R⁶ is independently cyclopropyl and R⁷ is halo;
each R⁶ is independently C₁-C₆ alkyl, and R⁷ is C₁-C₆ alkoxy optionally substituted with one or more halo;
each R⁶ is independently C₁-C₆ alkyl, and R⁷ is C₁-C₆ alkoxy;
each R⁶ is independently C₁-C₆ alkyl, and R⁷ is C₁-C₆ alkoxy substituted with one or more halo;
each R⁶ is independently halo, and R⁷ is C₁-C₆ haloalkyl;
each R⁶ is independently halo, and R⁷ is C₁-C₆ haloalkoxy;
each R⁶ is independently C₁-C₆ alkoxy; and R⁷ is halo;
each R⁶ is independently C₁-C₆ alkoxy; and R⁷ is chloro;
R⁷ is C₁-C₆ alkyl, and each R⁶ is independently C₁-C₆ alkyl optionally substituted with one or more halo;
R⁷ is C₁-C₆ alkyl, and each R⁶ is independently C₁-C₆ alkyl substituted with one or more halo;
R⁷ is C₁-C₆ alkyl, and each R⁶ is independently C₃-C₇ cycloalkyl;
R⁷ is C₁-C₆ alkyl, and each R⁶ is independently halo;
R⁷ is C₁-C₆ alkyl and each R⁶ is independently halo;
R⁷ is C₁-C₆ alkyl, and R⁶ is cyano;
R⁷ is C₃-C₇ cycloalkyl, and each R⁶ is independently C₃-C₇ cycloalkyl;
R⁷ is C₃-C₇ cycloalkyl, and each R⁶ is independently halo;
R⁷ is C₃-C₇ cycloalkyl and each R⁶ is independently halo;
R⁷ is C₁-C₆ alkyl, and each R⁶ is independently C₁-C₆ alkoxy optionally substituted with one or more halo;
R⁷ is C₁-C₆ alkyl, and each R⁶ is independently C₁-C₆ alkoxy;
R⁷ is C₁-C₆ alkyl, and each R⁶ is independently C₁-C₆ alkoxy substituted with one or more halo;
R⁷ is halo, and each R⁶ is independently C₁-C₆ haloalkyl;
R⁷ is halo, and each R⁶ is independently C₁-C₆ haloalkoxy;
R⁷ is C₁-C₆ alkoxy; and each R⁶ is independently halo;
R⁷ is C₁-C₆ alkoxy; and R⁶ is chloro;
two pairs, each of one R⁶ and one R⁷, are on adjacent atoms, and each pair of one R⁶ and one R⁷ taken together with the atoms connecting them form a C₄-C₈ aliphatic carbocyclic ring; and one R⁷ is halo;
two pairs, each of one R⁶ and one R⁷, are on adjacent atoms, and each pair of one R⁶ and one R⁷ taken together with the atoms connecting them form a C₄-C₈ aliphatic carbocyclic ring; and one R⁷ is cyano;
two pairs, each of one R⁶ and one R⁷, are on adjacent atoms, and each pair of one R⁶ and one R⁷ taken together with the atoms connecting them form a C₄-C₆ aliphatic carbocyclic ring optionally substituted with one or more hydroxy, oxo, or C₁-C₆ alkyl; and one R⁷ is halo or cyano;
two pairs, each of one R⁶ and one R⁷, are on adjacent atoms, and each pair of one R⁶ and one R⁷ taken together with the atoms connecting them form a 5-to-6-membered heterocyclic ring containing 1 heteroatom independently selected from O, N, and S, wherein the heterocyclic ring optionally substituted with one or more hydroxy, oxo, or C₁-C₆ alkyl; and one R⁷ is halo or cyano; or
two pairs, each of one R⁶ and one R⁷, are on adjacent atoms, one pair of one R⁶ and one R⁷ taken together with the atoms connecting them form a C₄-C₆ aliphatic carbocyclic ring optionally substituted with one or more hydroxy, oxo, or C₁-C₆ alkyl, and the other pair of one R⁶ and one R⁷ taken together with the atoms connecting them form a 5-to-6-membered heterocyclic ring containing 1 heteroatom independently selected from O, N, and S, wherein the heterocyclic ring optionally substituted with one or more hydroxy, oxo, or C₁-C₆ alkyl; and one R⁷ is halo or cyano.

In some embodiments, of the compound of formula AA,

the substituted ring B is

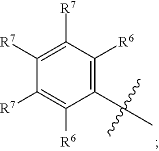

and R⁶ and Ware one of the following combinations:
each R⁶ is isopropyl; and each R⁷ is methyl;
each R⁶ is isopropyl; and each R⁷ is isopropyl;
each R⁶ is isopropyl; and each R⁷ is trifluoromethyl;
each R⁶ is isopropyl; and each R⁷ is cyclopropyl;
each R⁶ is isopropyl; and each R⁷ is chloro;
each R⁶ is isopropyl; and each R⁷ is fluoro;
each R⁶ is ethyl; and each R⁷ is fluoro;
each R⁶ is isopropyl; and each R⁷ is cyano;
each R⁶ is cyclopropyl; and each R⁷ is cyclopropyl;
each R⁶ is cyclopropyl; and each R⁷ is chloro;
each R⁶ is cyclopropyl; and each R⁷ is fluoro;
each R⁶ is isopropyl; and each R⁷ is methoxy;
each R⁶ is isopropyl; and each R⁷ is trifluoromethoxy;
each R⁶ is chloro; and each R⁷ is trifluoromethyl;
each R⁶ is chloro; and each R⁷ is trifluoromethoxy;
each R⁷ is isopropyl; and each R⁶ is methyl;
each R⁷ is isopropyl; and each R⁶ is trifluoromethyl;
each R⁷ is isopropyl; and each R⁶ is cyclopropyl;
each R⁷ is isopropyl; and each R⁶ is chloro;
each R⁷ is ethyl; and each R⁶ is fluoro;
each R⁷ is isopropyl; and each R⁶ is cyano;
each R⁷ is cyclopropyl; and each R⁶ is cyclopropyl;
each R⁷ is cyclopropyl; and each R⁶ is chloro;
each R⁷ is cyclopropyl; and each R⁶ is fluoro;
each R⁷ is isopropyl; and each R⁶ is methoxy;
each R⁷ is isopropyl; and each R⁶ is trifluoromethoxy;
each R⁷ is chloro; and each R⁶ is trifluoromethyl;
each R⁷ is chloro; and each R⁶ is trifluoromethoxy;
each R⁶ is isopropyl; two R⁷ are fluoro; and one R⁷ is chloro;
two pairs, each of one R⁶ and one R⁷, are on adjacent atoms, and each pair of one R⁶ and one R⁷ taken together with the atoms connecting them form a C₅ aliphatic carbocyclic ring; and one R⁷ is chloro;
two pairs, each of one R⁶ and one R⁷, are on adjacent atoms, and each pair of one R⁶ and one R⁷ taken together with the atoms connecting them form a C₅ aliphatic carbocyclic ring; and one R⁷ is fluoro;
two pairs, each of one R⁶ and one R⁷, are on adjacent atoms, and each pair of one R⁶ and one R⁷ taken together with the atoms connecting them form a C₄ aliphatic carbocyclic ring optionally substituted with one or more hydroxy, oxo, or methyl; and one R⁷ is fluoro or chloro;
two pairs, each of one R⁶ and one R⁷, are on adjacent atoms, and each pair of one R⁶ and one R⁷ taken together with the atoms connecting them form a C₅ aliphatic carbocyclic ring optionally substituted with one or more hydroxy, oxo, or methyl; and one R⁷ is fluoro or chloro;
two pairs, each of one R⁶ and one R⁷, are on adjacent atoms, and each pair of one R⁶ and one R⁷ taken together with the atoms connecting them form a C₆ aliphatic carbocyclic ring optionally substituted with one or more hydroxy, oxo, or methyl; and one R⁷ is fluoro or chloro;
two pairs, each of one R⁶ and one R⁷, are on adjacent atoms, and each pair of one R⁶ and one R⁷ taken together with the atoms connecting them form a 5-membered heterocyclic ring containing 1 heteroatom independently selected from O, N, and S, wherein the heterocyclic ring is optionally substituted with one or more hydroxy, oxo, or methyl; and one R⁷ is fluoro or chloro;
two pairs, each of one R⁶ and one R⁷, are on adjacent atoms, and each pair of one R⁶ and one R⁷ taken together with the atoms connecting them form a 6-membered heterocyclic ring containing 1 heteroatom independently selected from O, N, and S, wherein the heterocyclic ring is optionally substituted with one or more hydroxy, oxo, or methyl; and one R⁷ is fluoro or chloro; or
two pairs, each of one R⁶ and one R⁷, are on adjacent atoms, one pair of one R⁶ and one R⁷ taken together with the atoms connecting them form a 5-membered heterocyclic ring containing 1 heteroatom independently selected from O, N, and S, wherein the heterocyclic ring is optionally substituted with one or more hydroxy, oxo, or methyl, and the other pair of one R⁶ and one R⁷ taken together with the atoms connecting them form a C₅ aliphatic carbocyclic ring optionally substituted with one or more hydroxy, oxo, or methyl; and one R⁷ is fluoro or chloro.

In some embodiments of Formula AA, the substituted ring B is

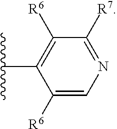

In certain of these embodiments, the R⁶ and R⁷ on adjacent atoms, taken together with the atoms connecting them, independently form C₄-C₇ (e.g., C₄ or C₅) carbocyclic ring or 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹. For example, the R⁶ and R⁷ on adjacent atoms, taken together with the atoms connecting them, independently form C₄-C₇ (e.g., C₅) carbocyclic ring. For example, the substituted ring B is

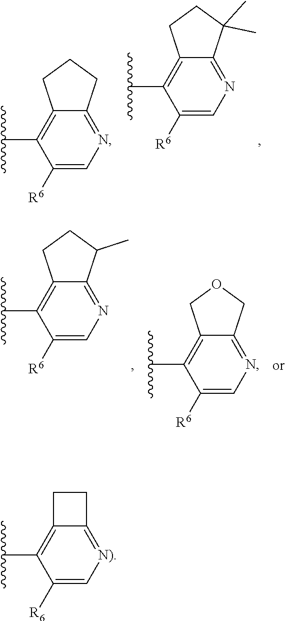

In certain embodiments (when

the substituted ring B is selected from:

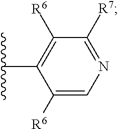

and the R⁶ and R⁷ on adjacent atoms, taken together with the atoms connecting them, independently form C₄-C₇ (e.g., C₄ or C₅) carbocyclic ring or 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹):

the remaining R⁶ is C₆-C₁₀ aryl or 5- to 10-membered heteroaryl, each of which is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl.

In certain of these embodiments, the remaining R⁶ is C₆-C₁₀ aryl or 5- to 10-membered heteroaryl optionally substituted with a substituent selected from halo, CN, C₁-C₆ alkyl, and C₁-C₆ alkoxy. For example, R⁶ is 5-6 membered heteroaryl (e.g., pyridinyl (e.g., pyridin-4-yl), pyrimidinyl, pyridazinyl, oxazolyl, or thiazolyl) optionally substituted with a substituent selected from halo, CN, C₁-C₆ alkyl, and C₁-C₆ alkoxy.

As a non-limiting example of the foregoing embodiments, substituted ring B is selected from:

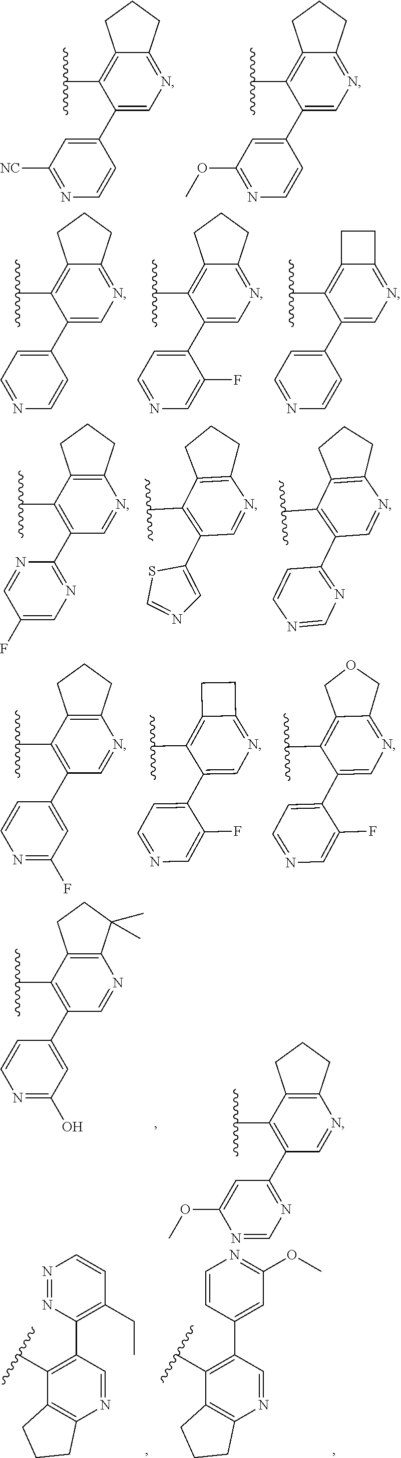

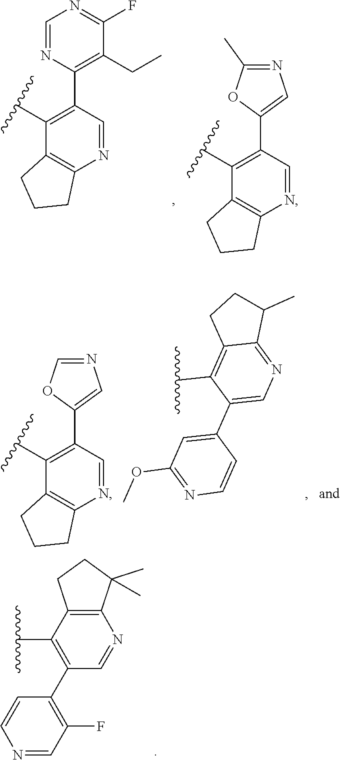

In certain embodiments (when

the substituted ring B is

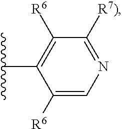

one R⁶ is C₆-C₁₀ aryl or 5- to 10-membered heteroaryl, each of which is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl.

In certain of these embodiments, one R⁶ is C₆-C₁₀ aryl or 5- to 10-membered heteroaryl optionally substituted with a substituent selected from halo, CN, C₁-C₆ alkyl, and C₁-C₆ alkoxy. For example, R⁶ is 5-6 membered heteroaryl (e.g., pyridinyl (e.g., pyridin-4-yl), pyrimidinyl, pyridazinyl, oxazolyl, or thiazolyl) optionally substituted with a substituent selected from halo, CN, C₁-C₆ alkyl, and C₁-C₆ alkoxy.

In certain embodiments (when

the substituted ring B is

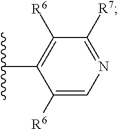

and one R⁶ is C₆-C₁₀ aryl or 5- to 10-membered heteroaryl, each of which is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl):

the remaining R⁶ and R⁷ are independently selected from the group consisting of cyano, halo, C₁-C₆ alkyl, C₁-C₆ haloalkyl, C₁-C₆ alkoxy, and C₃-C₇ cycloalkyl.

As non-limiting examples of the foregoing embodiments, B is:

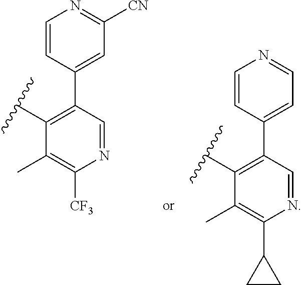 [SMILES: *c1c(-c2ccnc(C#N)c2)cnc(C(F)(F)F)c1C ; *c1c(-c2ccncc2)cnc(C2CC2)c1C]

In some embodiments of Formula AA, the substituted ring B is

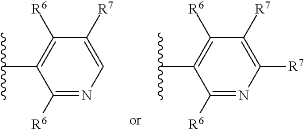

In certain of these embodiments, the R⁶ and R⁷ on adjacent atoms, taken together with the atoms connecting them, independently form C₄-C₇ (e.g., C₄ or C₅) carbocyclic ring or 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹. For example, the R⁶ and R⁷ on adjacent atoms, taken together with the atoms connecting them, independently form C₄-C₇ (e.g., C₅) carbocyclic ring. For example, the substituted ring B is

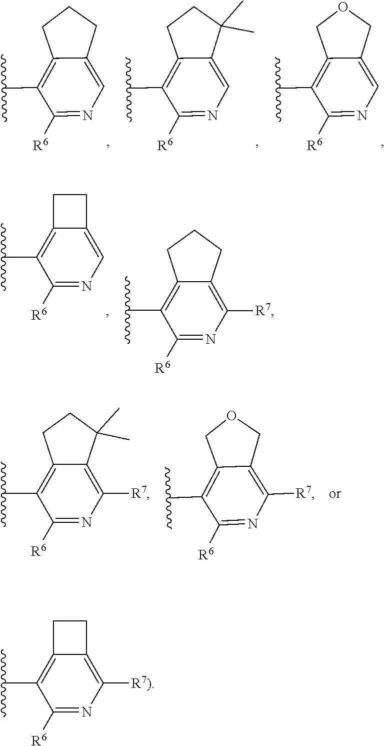

In certain embodiments (when

the substituted ring B is

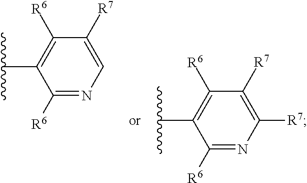

and the R⁶ and R⁷ on adjacent atoms, taken together with the atoms connecting them, independently form C₄-C₇ (e.g., C₄ or C₅) carbocyclic ring or 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹):

the remaining R⁶ is C₆-C₁₀ aryl or 5- to 10-membered heteroaryl, each of which is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl.

In certain of these embodiments, the remaining R⁶ is C₆-C₁₀ aryl or 5- to 10-membered heteroaryl optionally substituted with a substituent selected from halo, CN, C₁-C₆ alkyl, and C₁-C₆ alkoxy. For example, R⁶ is 5-6 membered heteroaryl (e.g., pyridinyl (e.g., pyridin-4-yl), pyrimidinyl, pyridazinyl, oxazolyl, or thiazolyl) optionally substituted with a substituent selected from halo, CN, C₁-C₆ alkyl, and C₁-C₆ alkoxy.

As a non-limiting example of the foregoing embodiments, substituted ring B is selected from:

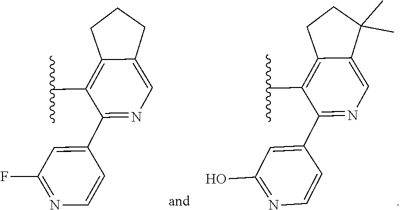 [SMILES: *c1c(-c2ccnc(F)c2)ncc2c1CCC2 ; *c1c(-c2ccnc(O)c2)ncc2c1CCC2(C)C]

In certain embodiments (when the substituted ring B is

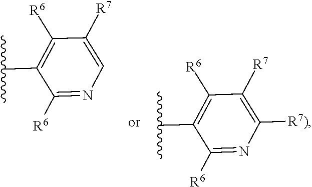

one R⁶ is C₆-C₁₀ aryl or 5- to 10-membered heteroaryl, each of which is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl.

In certain of these embodiments, one R⁶ is C₆-C₁₀ aryl or 5- to 10-membered heteroaryl optionally substituted with a substituent selected from halo, CN, C₁-C₆ alkyl, and C₁-C₆ alkoxy. For example, R⁶ is 5-6 membered heteroaryl (e.g., pyridinyl (e.g., pyridin-4-yl), pyrimidinyl, pyridazinyl, oxazolyl, or thiazolyl) optionally substituted with a substituent selected from halo, CN, C₁-C₆ alkyl, and C₁-C₆ alkoxy.

In certain embodiments (when

the substituted ring B is

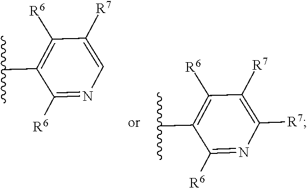

and one R⁶ is C₆-C₁₀ aryl or 5- to 10-membered heteroaryl, each of which is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl):

the remaining R⁶ and each R⁷ are independently selected from the group consisting of cyano, halo, C₁-C₆ alkyl, C₁-C₆ haloalkyl, C₁-C₆ alkoxy, and C₃-C₇ cycloalkyl.

As non-limiting examples of the foregoing embodiments, B is:

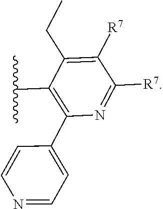

In some embodiments of Formula AA, the substituted ring B is

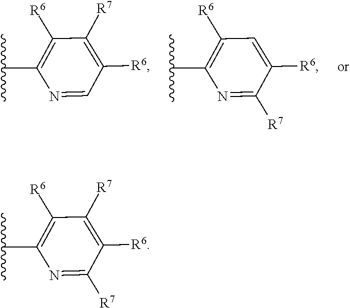

In certain of these embodiments, one pair of R⁶ and R⁷ on adjacent atoms, taken together with the atoms connecting them, independently form C₄-C₇ (e.g., C₄ or C₅) carbocyclic ring or 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹. For example, the R⁶ and R⁷ on adjacent atoms, taken together with the atoms connecting them, independently form C₄-C₇ (e.g., C₅) carbocyclic ring. For example, the substituted ring B is

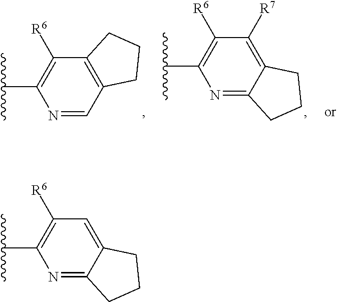

In certain embodiments (when

the substituted ring B is

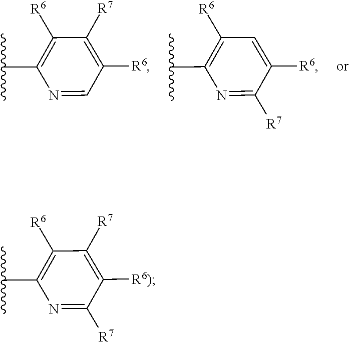

and one pair of R⁶ and R⁷ on adjacent atoms, taken together with the atoms connecting them, independently form C₄-C₇ (e.g., C₄ or C₅) carbocyclic ring or 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, ═NR¹⁰, COOC₁-C₆ alkyl, C₆-C₁₀ aryl, and CONR⁸R⁹):

the remaining R⁶ is C₆-C₁₀ aryl or 5- to 10-membered heteroaryl, each of which is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl.

In certain of these embodiments, the remaining R⁶ is C₆-C₁₀ aryl or 5- to 10-membered heteroaryl optionally substituted with a substituent selected from halo, CN, C₁-C₆ alkyl, and C₁-C₆ alkoxy. For example, R⁶ is 5-6 membered heteroaryl (e.g., pyridinyl (e.g., pyridin-4-yl), pyrimidinyl, pyridazinyl, oxazolyl, or thiazolyl) optionally substituted with a substituent selected from halo, CN, C₁-C₆ alkyl, and C₁-C₆ alkoxy.

As a non-limiting example of the foregoing embodiments, substituted ring B is selected from:

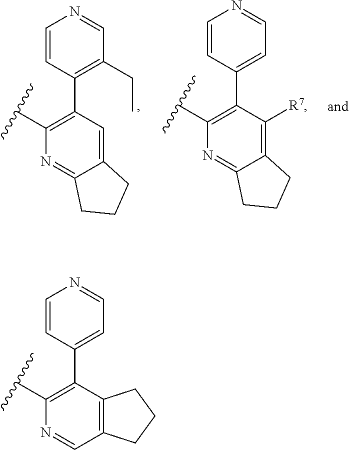

(e.g., R⁷ is cyano or halo (e.g., halo such as F)).

Non-Limiting Combinations

In some embodiments, when ring A is phenyl, then R¹ and R² are each independently selected from C₃ alkyl, C₅-C₆ alkyl, C₁-C₆ haloalkyl, C₁-C₆ alkoxy, C₁-C₆ haloalkoxy, F, I, CN, NO₂, COC₂-C₆ alkyl, CO-C₆-C₁₀ aryl, CO(5- to 10-membered heteroaryl), CO₂C₁-C₆ alkyl, CO₂C₃-C₈ cycloalkyl, OCOC₂-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, NHCOC₁-C₆ alkyl, NHCOC₆-C₁₀ aryl, NHCO(5- to 10-membered heteroaryl), NHCO(3- to 7-membered heterocycloalkyl), NHCOC₂-C₆ alkynyl, NHCOOCC₁-C₆ alkyl, NH—(C═NR¹³)NR¹¹R¹², CONR⁸R⁹, SF₅, SC₁-C₆ alkyl, S(O₂)C₁-C₆ alkyl, S(O₂)NR¹¹R¹², S(O)C₁-C₆ alkyl, C₃-C₇ cycloalkyl, and 3- to 7-membered heterocycloalkyl, wherein the C₁-C₆ alkyl, C₁-C₆ haloalkyl, C₃-C₇ cycloalkyl and 3- to 7-membered heterocycloalkyl are optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;

wherein each C₁-C₆ alkyl substituent and each C₁-C₆ alkoxy substituent of the R¹ or R² C₃-C₇ cycloalkyl or of the R¹ or R² 3- to 7-membered heterocycloalkyl is further optionally independently substituted with one to three hydroxy, halo, NR⁸R⁹, or oxo;

wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;

or one pair of R¹ and R² on adjacent atoms, taken together with the atoms connecting them, independently form one monocyclic or bicyclic C₄ or C₆-C₁₂ carbocyclic ring or one monocyclic or bicyclic 5- to-12-membered heterocyclic ring containing 1-3 heteroatoms independently selected from O, N, and S, wherein the carbocyclic ring or heterocyclic ring is optionally independently substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR⁸R⁹, ═NR¹⁰, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR⁸R⁹, wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR⁸R⁹, ═NR¹⁰, COOC₁-C₆ alkyl, C₆-C₁₀ aryl, and CONR⁸R⁹; and

R⁶ and R⁷ are each independently selected from a C₂-C₆ alkyl, C₂-C₆ haloalkyl, C₂-C₆ alkoxy, C₁-C₆ haloalkoxy, I, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, C₃-C₁₀ cycloalkyl and 3- to 10-membered heterocycloalkyl, and C₂-C₆ alkenyl,

wherein R⁶ and R⁷ are each optionally substituted with one or more substituents independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), NHCOC₂-C₆ alkynyl, C₆-C₁₀ aryloxy, and S(O₂)C₁-C₆ alkyl; and wherein the C₁-C₆ alkyl or C₁-C₆ alkoxy that R⁶ or R⁷ is substituted with is optionally substituted with one or more hydroxyl, C₆-C₁₀ aryl or NR⁸R⁹, or wherein R⁶ or R⁷ is optionally fused to a five- to -seven-membered carbocyclic ring or heterocyclic ring containing one or two heteroatoms independently selected from oxygen, sulfur and nitrogen;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;

or one pair of R⁶ and R⁷ on adjacent atoms, taken together with the atoms connecting them, independently form at least one C₄-C₈ carbocyclic ring or at least one 5- to 8-membered heterocyclic ring containing 1 or 2 heteroatoms and/or heteroatomic groups independently selected from O, NH, NR¹³, S, S(O), and S(O)₂, wherein the selected heteroatoms and/or heteroatomic groups are cumulative with the nitrogen atoms present in ring B, and wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹.

In some embodiments, R¹ and R² are each independently selected from C₃ alkyl, CS-C₆ alkyl, C₁-C₆ haloalkyl, C₁-C₆ alkoxy, C₁-C₆ haloalkoxy, F, I, CN, NO₂, COC₂-C₆ alkyl, CO-C₆-C₁₀ aryl, CO(5- to 10-membered heteroaryl), CO₂C₁-C₆ alkyl, CO₂C₃-C₈ cycloalkyl, OCOC₂-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, NHCOC₁-C₆ alkyl, NHCOC₆-C₁₀ aryl, NHCO(5- to 10-membered heteroaryl), NHCO(3- to 7-membered heterocycloalkyl), NHCOC₂-C₆ alkynyl, NHCOOCC₁-C₆ alkyl, NH—(C═NR¹³)NR¹¹R¹², CONR⁸R⁹, SF₅, SC₁-C₆ alkyl, S(O₂)C₁-C₆ alkyl, S(O₂)NR¹¹R¹², S(O)C₁-C₆ alkyl, C₃-C₇ cycloalkyl, and 3- to 7-membered heterocycloalkyl, wherein the C₁-C₆ alkyl, C₁-C₆ haloalkyl, C₃-C₇ cycloalkyl and 3- to 7-membered heterocycloalkyl are optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;
wherein each C₁-C₆ alkyl substituent and each C₁-C₆ alkoxy substituent of the R¹ or R² C₃-C₇ cycloalkyl or of the R¹ or R² 3- to 7-membered heterocycloalkyl is further optionally independently substituted with one to three hydroxy, halo, NR⁸R⁹, or oxo;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;

or one pair of R¹ and R² on adjacent atoms, taken together with the atoms connecting them, independently form one monocyclic or bicyclic C₄ or C₆-C₁₂ carbocyclic ring or one monocyclic or bicyclic 5- to-12-membered heterocyclic ring containing 1-3 heteroatoms independently selected from O, N, and S, wherein the carbocyclic ring or heterocyclic ring is optionally independently substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR⁸R⁹,═NR¹⁰,CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR⁸R⁹, wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR⁸R⁹, ═NR¹⁰, COOC₁-C₆ alkyl, C₆-C₁₀ aryl, and CONR⁸R⁹; and

R⁶ and R⁷ are each independently selected from a C₂-C₆ alkyl, C₂-C₆ haloalkyl, C₂-C₆ alkoxy, C₁-C₆ haloalkoxy, I, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, C₃-C₁₀ cycloalkyl and 3- to 10-membered heterocycloalkyl, and C₂-C₆ alkenyl,

wherein R⁶ and R⁷ are each optionally substituted with one or more substituents independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), NHCOC₂-C₆ alkynyl, C₆-C₁₀ aryloxy, and S(O₂)C₁-C₆ alkyl; and wherein the C₁-C₆ alkyl or C₁-C₆ alkoxy that R⁶ or R⁷ is substituted with is optionally substituted with one or more hydroxyl, C₆-C₁₀ aryl or NR⁸R⁹, or wherein R⁶ or R⁷ is optionally fused to a five- to -seven-membered carbocyclic ring or heterocyclic ring containing one or two heteroatoms independently selected from oxygen, sulfur and nitrogen;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;

or one pair of R⁶ and R⁷ on adjacent atoms, taken together with the atoms connecting them, independently form at least one C₄-C₈ carbocyclic ring or at least one 5- to 8-membered heterocyclic ring containing 1 or 2 heteroatoms and/or heteroatomic groups independently selected from O, NH, NR¹³, S, S(O), and S(O)₂, wherein the selected heteroatoms and/or heteroatomic groups are cumulative with the nitrogen atoms present in ring B, and wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹.

In some embodiments of the compound of Formula AA, when ring A is pyridyl, then R¹ and R² are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO-C₆-C₁₀ aryl, CO(5- to 10-membered heteroaryl), CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₂-C₆ alkyl, N(C₁-C₆ alkyl)₂, NHCOC₁-C₆ alkyl, NHCOC₆-C₁₀ aryl, NHCO(5- to 10-membered heteroaryl), NHCO(3- to 7-membered heterocycloalkyl), NHCOC₂-C₆ alkynyl, NHCOOCC₁-C₆ alkyl, NH—(C═NR¹³)NR¹¹R¹², CONR⁸R⁹, SF₅, SC₁-C₆ alkyl, S(O₂)C₁-C₆ alkyl, S(O₂)NR¹¹R¹², S(O)C₁-C₆ alkyl, C₃-C₇ cycloalkyl, and 3- to 7-membered heterocycloalkyl, wherein the C₁-C₆ alkyl, C₁-C₆ haloalkyl, C₃-C₇ cycloalkyl, and 3- to 7-membered heterocycloalkyl are optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 5-membered heterocycloalkyl, 5-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;
wherein each C₁-C₆ alkyl substituent and each C₁-C₆ alkoxy substituent of the R¹ or R² C₃-C₇ cycloalkyl or of the R¹ or R² 3- to 7-membered heterocycloalkyl is further optionally independently substituted with one to three hydroxy, halo, NR⁸R⁹, or oxo;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;

or one pair of R¹ and R² on adjacent atoms, taken together with the atoms connecting them, independently form one monocyclic or bicyclic C₄-C₁₂ carbocyclic ring or one monocyclic or bicyclic 5- to-12-membered heterocyclic ring containing 1-3 heteroatoms independently selected from O, N, and S, wherein the carbocyclic ring or heterocyclic ring is optionally independently substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR⁸R⁹, ═NR¹⁰, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR⁸R⁹, wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR⁸R⁹, ═NR¹⁰, COOC₁-C₆ alkyl, C₆-C₁₀ aryl, and CONR⁸R⁹; and R⁶ and R⁷ are each independently selected from a C₂-C₆ alkyl, C₂-C₆ haloalkyl, C₂-C₆ alkoxy, C₁-C₆ haloalkoxy, I, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, C₃-C₁₀ cycloalkyl and 3- to 10-membered heterocycloalkyl, and C₂-C₆ alkenyl,

wherein R⁶ and R⁷ are each optionally substituted with one or more substituents independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), NHCOC₂-C₆ alkynyl, C₆-C₁₀ aryloxy, and S(O₂)C₁-C₆ alkyl; and wherein the C₁-C₆ alkyl or C₁-C₆ alkoxy that R⁶ or R⁷ is substituted with is optionally substituted with one or more hydroxyl, C₆-C₁₀ aryl or NR⁸R⁹, or wherein R⁶ or R⁷ is optionally fused to a five- to -seven-membered carbocyclic ring or heterocyclic ring containing one or two heteroatoms independently selected from oxygen, sulfur and nitrogen;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;

or one pair of R⁶ and R⁷ on adjacent atoms, taken together with the atoms connecting them, independently form at least one C₄-C₈ carbocyclic ring or at least one 5- to 8-membered heterocyclic ring containing 1 or 2 heteroatoms and/or heteroatomic groups independently selected from O, NH, NR¹³, S, S(O), and S(O)₂, wherein the selected heteroatoms and/or heteroatomic groups are cumulative with the nitrogen atoms present in ring B, and wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹.

In some embodiments of the compound of Formula AA, R¹ and R² are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO-C₆-C₁₀ aryl, CO(5- to 10-membered heteroaryl), CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₂-C₆ alkyl, N(C₁-C₆ alkyl)₂, NHCOC₁-C₆ alkyl, NHCOC₆-C₁₀ aryl, NHCO(5- to 10-membered heteroaryl), NHCO(3- to 7-membered heterocycloalkyl), NHCOC₂-C₆ alkynyl, NHCOOCC₁-C₆ alkyl, NH—(C═NR¹³)NR¹¹R¹², CONR⁸R⁹, SF₅, SC₁-C₆ alkyl, S(O₂)C₁-C₆ alkyl, S(O₂)NR¹¹R¹², S(O)C₁-C₆ alkyl, C₃-C₇ cycloalkyl, and 3- to 7-membered heterocycloalkyl,

wherein the C₁-C₆ alkyl, C₁-C₆ haloalkyl, C₃-C₇ cycloalkyl, and 3- to 7-membered heterocycloalkyl are optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 5-membered heterocycloalkyl, 5-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;
wherein each C₁-C₆ alkyl substituent and each C₁-C₆ alkoxy substituent of the R¹ or R² C₃-C₇ cycloalkyl or of the R¹ or R² 3- to 7-membered heterocycloalkyl is further optionally independently substituted with one to three hydroxy, halo, NR⁸R⁹, or oxo;

wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;

or one pair of R¹ and R² on adjacent atoms, taken together with the atoms connecting them, independently form one monocyclic or bicyclic C₄-C₁₂ carbocyclic ring or one monocyclic or bicyclic 5- to-12-membered heterocyclic ring containing 1-3 heteroatoms independently selected from O, N, and S, wherein the carbocyclic ring or heterocyclic ring is optionally independently substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR⁸R⁹, ═NR¹⁰, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR⁸R⁹, wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR⁸R⁹, ═NR¹⁰, COOC₁-C₆ alkyl, C₆-C₁₀ aryl, and CONR⁸R⁹; and R⁶ and R⁷ are each independently selected from a C₂-C₆ alkyl, C₂-C₆ haloalkyl, C₂-C₆ alkoxy, C₁-C₆ haloalkoxy, I, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, C₃-C₁₀ cycloalkyl and 3- to 10-membered heterocycloalkyl, and C₂-C₆ alkenyl,

wherein R⁶ and R⁷ are each optionally substituted with one or more substituents independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), NHCOC₂-C₆ alkynyl, C₆-C₁₀ aryloxy, and S(O₂)C₁-C₆ alkyl; and wherein the C₁-C₆ alkyl or C₁-C₆ alkoxy that R⁶ or R⁷ is substituted with is optionally substituted with one or more hydroxyl, C₆-C₁₀ aryl or NR⁸R⁹, or wherein R⁶ or R⁷ is optionally fused to a five- to -seven-membered carbocyclic ring or heterocyclic ring containing one or two heteroatoms independently selected from oxygen, sulfur and nitrogen;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;

or one pair of R⁶ and R⁷ on adjacent atoms, taken together with the atoms connecting them, independently form at least one C₄-C₈ carbocyclic ring or at least one 5- to 8-membered heterocyclic ring containing 1 or 2 heteroatoms and/or heteroatomic groups independently selected from O, NH, NR¹³, S, S(O), and S(O)₂, wherein the selected heteroatoms and/or heteroatomic groups are cumulative with the nitrogen atoms present in ring B, and wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹.

In some embodiments, R¹ and R² are each independently selected from C₃ alkyl, C₅-C₆ alkyl, C₁-C₆ haloalkyl, C₁-C₆ alkoxy, C₁-C₆ haloalkoxy, F, I, CN, NO₂, COC₂-C₆ alkyl, CO-C₆-C₁₀ aryl, CO(5- to 10-membered heteroaryl), CO₂C₁-C₆ alkyl, CO₂C₃-C₈ cycloalkyl, OCOC₂-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₂-C₆ alkyl, N(C₁-C₆ alkyl)₂, NHCOC₁-C₆ alkyl, NHCOC₆-C₁₀ aryl, NHCO(5- to 10-membered heteroaryl), NHCO(3- to 7-membered heterocycloalkyl), NHCOC₂-C₆ alkynyl, NHCOOCC₁-C₆ alkyl, NH—(C═NR¹³)NR¹¹R¹², CONR⁸R⁹, SF₅, SC₁-C₆ alkyl, S(O₂)C₁-C₆ alkyl, S(O₂)NR¹¹R¹², S(O)C₁-C₆ alkyl, C₃-C₇ cycloalkyl, and 3- to 7-membered heterocycloalkyl, wherein the C₁-C₆ alkyl, C₁-C₆ haloalkyl, C₃-C₇ cycloalkyl and 3- to 7-membered heterocycloalkyl are optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), and NHCOC₂-C₆ alkynyl;
wherein each C₁-C₆ alkyl substituent and each C₁-C₆ alkoxy substituent of the R¹ or R² C₃-C₇ cycloalkyl or of the R¹ or R² 3- to 7-membered heterocycloalkyl is further optionally independently substituted with one to three hydroxy, halo, NR⁸R⁹, or oxo;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;

or one pair of R¹ and R² on adjacent atoms, taken together with the atoms connecting them, independently form one monocyclic or bicyclic C₄ or C₆-C₁₂ carbocyclic ring or one monocyclic or bicyclic 5- to-12-membered heterocyclic ring containing 1-3 heteroatoms independently selected from O, N, and S, wherein the carbocyclic ring or heterocyclic ring is optionally independently substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR⁸R⁹, ═NR¹⁰, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR⁸R⁹, wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR⁸R⁹, ═NR¹⁰, COOC₁-C₆ alkyl, C₆-C₁₀ aryl, and CONR⁸R⁹; and

R⁶ and R⁷ are each independently selected from a C₂-C₆ alkyl, C₂-C₆ haloalkyl, C₂-C₆ alkoxy, C₁-C₆ haloalkoxy, I, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, C₃-C₁₀ cycloalkyl and 3- to 10-membered heterocycloalkyl, and C₂-C₆ alkenyl,

wherein R⁶ and R⁷ are each optionally substituted with one or more substituents independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 5-membered heterocycloalkyl, 5-membered heteroaryl, OCOC₁-C₆ alkyl, OCOC₆-C₁₀ aryl, OCO(5- to 10-membered heteroaryl), OCO(3- to 7-membered heterocycloalkyl), NHCOC₁-C₆ alkyl, NHCOC₆-C₁₀ aryl, NHCO(5- to 10-membered heteroaryl), NHCO(3- to 7-membered heterocycloalkyl), NHCOC₂-C₆ alkynyl, C₆-C₁₀ aryloxy, and S(O₂)C₁-C₆ alkyl; and

wherein the C₁-C₆ alkyl or C₁-C₆ alkoxy that R⁶ or R⁷ is substituted with is optionally substituted with one or more hydroxyl, C₆-C₁₀ aryl or NR⁸R⁹, or wherein R⁶ or R⁷ is optionally fused to a five- to seven-membered carbocyclic ring or heterocyclic ring containing one or two heteroatoms independently selected from oxygen, sulfur and nitrogen;
wherein the 3- to 7-membered heterocycloalkyl, C₆-C₁₀ aryl, 5- to 10-membered heteroaryl, NHCOC₆-C₁₀ aryl, NHCO(5- to 10-membered heteroaryl) and NHCO(3- to 7-membered heterocycloalkyl) are optionally substituted with one or more substituents independently selected from halo, C₁-C₆ alkyl, and OC₁-C₆ alkyl;

or one pair of R⁶ and R⁷ on adjacent atoms, taken together with the atoms connecting them, independently form at least one C₄-C₈ carbocyclic ring or at least one 5- to 8-membered heterocyclic ring containing 1 or 2 heteroatoms and/or heteroatomic groups independently selected from O, NH, NR¹³, S, S(O), and S(O)₂, wherein the selected heteroatoms and/or heteroatomic groups are cumulative with the nitrogen atoms present in ring B, and wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹.

### [Combination 1]

In some embodiments, the optionally substituted ring A is

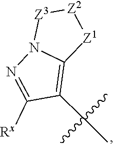

wherein R^{x} is selected from the group consisting of H and C₁-C₆ alkyl (e.g., methyl); Z¹ is selected from the group consisting of O, NH, and —CH₂— optionally substituted with 1-2 R²⁰; Z² is selected from the group consisting of NH and —CH₂— optionally substituted with 1-2 R²⁰; Z³ is selected from the group consisting of —CH₂— optionally substituted with 1-2 R²⁰, —CH₂CH₂— optionally substituted with 1-2 R²⁰, and —CH₂CH₂CH₂— optionally substituted with 1-2 R²⁰; R²⁰ is selected from the group consisting of hydroxy, halo (e.g., fluoro), oxo, C₁-C₆ alkyl (e.g., methyl or ethyl) optionally substituted with one R²¹, C₁-C₆ alkoxy (e.g., methoxy, ethoxy, or isopropoxy) optionally substituted with one R²¹, NR⁸R⁹, 3- to 10-membered heterocycloalkyl (e.g., azetidinyl or pyrrolidinyl) optionally substituted with one R²¹, or one pair of R²⁰ on the same atom, taken together with the atom connecting them, independently forms a monocyclic C₃-C₄ carbocyclic ring or a monocyclic 3- to 4-membered heterocyclic ring containing 1 O atom optionally substituted with OS(O)₂Ph; R²¹ is selected from the group consisting of halo (e.g., fluoro), NR⁸R⁹, C₂-C₆ alkynyl (e.g., ethynyl), and C₁-C₆ alkoxy (e.g., methoxy); R⁸ and R⁹ at each occurrence is independently selected from hydrogen, C₁-C₆ alkyl (e.g., methyl or ethyl), COR¹³, and CO₂R¹³; R¹³ is selected from the group consisting of: C₁-C₆ alkyl (e.g., methyl or t-butyl) and C₁-C₆ haloalkyl (e.g., trifluoromethyl); and

the substituted ring B is selected from:

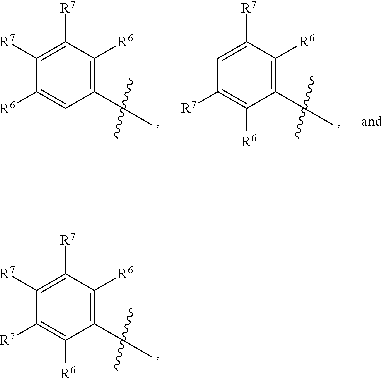

wherein

two pairs, each of one R⁶ and one R⁷, are on adjacent atoms, and each pair of one R⁶ and one R⁷ taken together with the atoms connecting them independently form a C₄-C₇ carbocyclic ring or a 5- to 7-membered heterocyclic ring containing 1 or 2 heteroatoms independently selected from O, N, and S, wherein each carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹; and

the remaining R⁷ that is not taken with a R⁶ on adjacent atoms to form a C₄-C₇ carbocyclic ring or a 5- to 7-membered heterocyclic ring is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy.

### [Combination 2]

In some embodiments, the optionally substituted ring A is

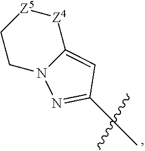

wherein Z⁴ is selected from the group consisting of —CH₂—, —C(O)—, and NH; Z⁵ is selected from the group consisting of O, NH, N—CH₃, and —CH₂—; and

the substituted ring B is selected from:

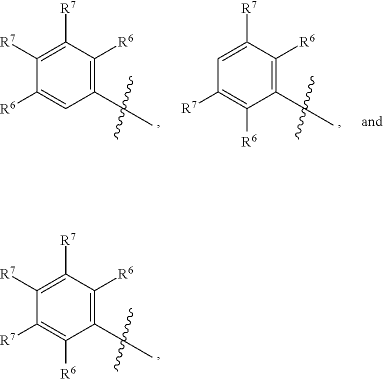

wherein

two pairs, each of one R⁶ and one R⁷, are on adjacent atoms, and each pair of one R⁶ and one R⁷ taken together with the atoms connecting them independently form a C₄-C₇ carbocyclic ring or a 5- to 7-membered heterocyclic ring containing 1 or 2 heteroatoms independently selected from O, N, and S, wherein each carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹; and

the remaining R⁷ that is not taken with a R⁶ on adjacent atoms to form a C₄-C₇ carbocyclic ring or a 5- to 7-membered heterocyclic ring is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy.

In some embodiments of [Combination 1] and [Combination 2], the substituted ring B is

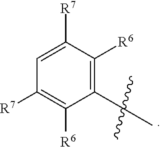

In some embodiments of [Combination 1] and [Combination 2], the substituted ring B is

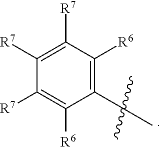

In some embodiments of [Combination 1] and [Combination 2], one pair of R⁶ and R⁷ taken together with the atoms connecting them independently form a C₅ carbocyclic ring, wherein the carbocyclic ring is optionally substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In certain of these embodiments, the other pair of R⁶ and R⁷ taken together with the atoms connecting them independently form a C₅ carbocyclic ring, wherein the carbocyclic ring is optionally substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

As non-limiting examples of the foregoing embodiments, the substituted ring B is selected from:

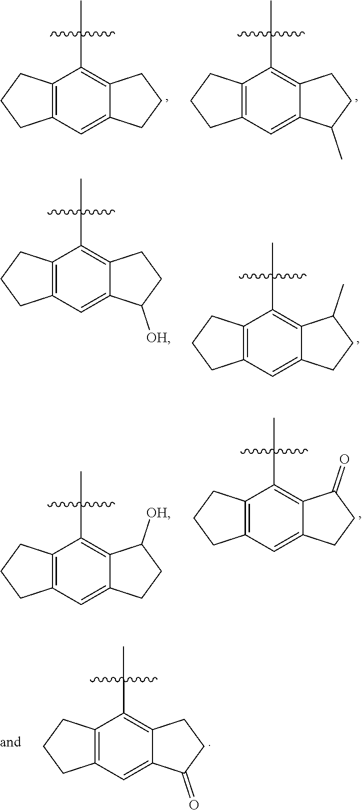

As further non-limiting examples, the substituted ring B is

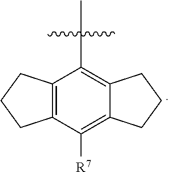

In certain embodiments of [Combination 1] and [Combination 2] (when one pair of R⁶ and R⁷ taken together with the atoms connecting them independently form a C₅ carbocyclic ring, wherein the carbocyclic ring is optionally substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹), the other pair of R⁶ and R⁷ taken together with the atoms connecting them independently form a C₄ carbocyclic ring, wherein the carbocyclic ring is optionally substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹ (e.g., the substituted ring B is:

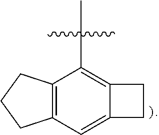 [SMILES: *c1c2c(cc3c1CC3)CCC2]

In some embodiments of [Combination 1] and [Combination 2], one pair of R⁶ and R⁷ taken together with the atoms connecting them independently form a C₄ carbocyclic ring, wherein the carbocyclic ring is optionally substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In certain of these embodiments, the other pair of R⁶ and R⁷ taken together with the atoms connecting them independently form a C₄ carbocyclic ring, wherein the carbocyclic ring is optionally substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹ (e.g., the substituted ring B is:

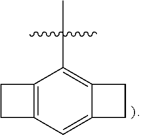 [SMILES: *c1c2c(cc3c1CC3)CC2]

In some embodiments of [Combination 1] and [Combination 2], the substituted ring B is

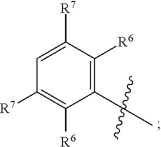

and one pair of R⁶ and R⁷ on adjacent atoms, taken together with the atoms connecting them, independently form one C₄-C₇ (e.g., C₅) carbocyclic ring; and each of the remaining R⁶ and R⁷ is independently selected from C₁-C₆ alkyl, C₁-C₆ haloalkyl, and C₃-C₇ cycloalkyl (e.g., the substituted ring B is

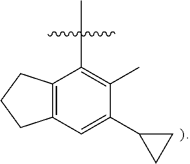 [SMILES: *c1c(C)c(C2CC2)cc2c1CCC2]

In some embodiments [Combination 1] and [Combination 2], the substituted ring B is

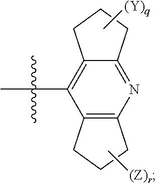

q is 0, 1, or 2; r is 0, 1, or 2; wherein each of Y and Z is independently selected from C₁-C₆ alkyl (e.g., methyl) and hydroxy; or wherein when two Y are attached to the same carbon, the two Y are taken together with the carbon they are attached to to form a cyclopropyl ring; or wherein when two Z are attached to the same carbon, the two Z are taken together with the carbon they are attached to form a cyclopropyl ring.

In some embodiments [Combination 1] and [Combination 2], the substituted ring B is

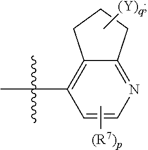

R⁷ is selected from C₁-C₆ alkyl (e.g., methyl, ethyl, or isopropyl), C₆-C₁₀ aryl (e.g., phenyl), and C₃-C₁₀ cycloalkyl (e.g., cyclopropyl); p is 0, 1, or 2; q is 0, 1, or 2; wherein each Y is independently selected from C₁-C₆ alkyl (e.g., methyl) and hydroxy; or when two Y are attached to the same carbon, the two Y are taken together with the carbon they are attached to to form a cyclopropyl ring.

In some embodiments [Combination 1] and [Combination 2], the substituted ring B is

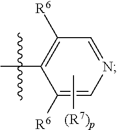

each R⁶ is independently selected from C₁-C₆ alkyl (e.g., isopropyl); each R⁷ is independently selected from halo (e.g., fluoro); p is 0 or 1.

In some embodiments [Combination 1] and [Combination 2], the substituted ring B is

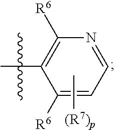

each R⁶ is independently selected from C₁-C₆ alkyl (e.g., isopropyl); each R⁷ is independently selected from halo (e.g., fluoro); p is 0 or 1.

In some embodiments [Combination 1] and [Combination 2], the substituted ring B is

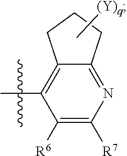

R⁶ is selected from C₁-C₆ alkyl (e.g., methyl, ethyl, or isopropyl) and C₃-C₁₀ cycloalkyl (e.g., cyclopropyl); R⁷ is selected from C₁-C₆ alkyl (e.g., methyl, ethyl, or isopropyl), C₁-C₆ haloalkyl (e.g., trifluoromethyl) and C₃-C₁₀ cycloalkyl (e.g., cyclopropyl or cyclobutyl); or R⁶ and R⁷, taken together with the atoms connecting them, independently form a C₅ carbocyclic ring optionally substituted with one or more C₁-C₆ alkyl (e.g., methyl); q is 0, 1, or 2; each Y is independently selected from C₁-C₆ alkyl (e.g., methyl); or when two Y are attached to the same carbon, the two Y are taken together with the carbon they are attached to to form a cyclopropyl ring.

In some embodiments of [Combination 1] and [Combination 2], L^{A} is a bond.

In some embodiments of [Combination 1] and [Combination 2], L^{A} is CH₂.

### [Combination 3]

In some embodiments, the compound of Formula AA is an unequal mixture (e.g., a non-racemic mixture when the sulfur is the only stereocenter in Formula AA) of:

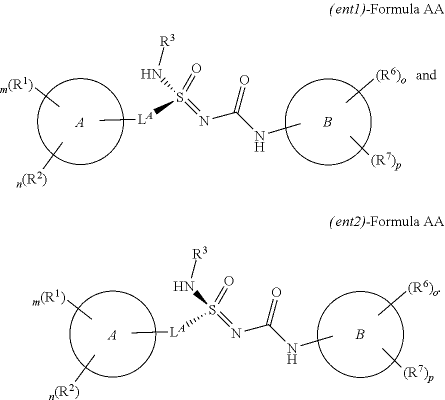

In certain of these embodiments, the mixture is enriched in (ent1)-Formula AA.

In certain of other embodiments, the mixture is enriched in (ent2)-Formula AA.

In certain embodiments, the compound is a compound of (ent1)-Formula AA that is substantially free (e.g., contains less than about 10% of, less than about 5% of, less than about 2% of, less than about 1%, less than about 0.5% of) of (ent2)-Formula AA.

In certain embodiments, the compound is a compound of (ent2)-Formula AA that is substantially free (e.g., contains less than about 10% of, less than about 5% of, less than about 2% of, less than about 1%, less than about 0.5% of) of (ent1)-Formula AA.

In some embodiments, the compound of Formula AA has Formula AA-CN:

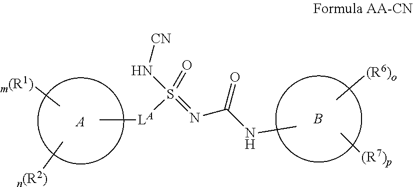

or a pharmaceutically acceptable salt thereof, wherein the variables in Formula AA-CN can be as defined anywhere herein.

In certain of these embodiments, the compound of Formula AA-CN is an unequal mixture (e.g., a non-racemic mixture when the sulfur is the only stereocenter in Formula AA-CN):

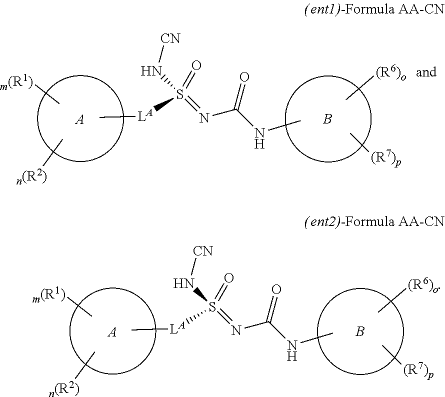

In certain of these embodiments, the mixture is enriched in (ent1)-Formula AA-CN.

In certain of other embodiments, the mixture is enriched in (ent2)-Formula AA-CN.

In certain embodiments, the compound is a compound of (ent1)-Formula AA-CN that is substantially free (e.g., contains less than about 10% of, less than about 5% of, less than about 2% of, less than about 1%, less than about 0.5% of) of (ent2)-Formula AA-CN.

In certain embodiments, the compound is a compound of (ent2)-Formula AA-CN that is substantially free (e.g., contains less than about 10% of, less than about 5% of, less than about 2% of, less than about 1%, less than about 0.5% of) of (ent1)-Formula AA-CN.

The inventors have discovered that in the corresponding N—H (R³═H) compounds, activity primarily resides in only one of the two sulfur enantiomers or epimers; however, surprisingly, when said hydrogen atom is replaced with a cyano group, activity resides primarily in the opposing enantiomer or epimer.

In some embodiments of [combination 3], the optionally substituted ring A is

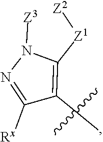

wherein R^{x} is selected from the group consisting of H and C₁-C₆ alkyl (e.g., methyl); Z¹ is selected from the group consisting of O, NH, and —CH₂— optionally substituted with 1-2 R²⁰; Z² is selected from the group consisting of NH and —CH₂— optionally substituted with 1-2 R²⁰; Z³ is selected from the group consisting of —CH₂— optionally substituted with 1-2 R²⁰, —CH₂CH₂— optionally substituted with 1-2 R²⁰, and —CH₂CH₂CH₂— optionally substituted with 1-2 R²⁰; R²⁰ is selected from the group consisting of hydroxy, halo (e.g., fluoro), oxo, C₁-C₆ alkyl (e.g., methyl or ethyl) optionally substituted with one R²¹, C₁-C₆ alkoxy (e.g., methoxy, ethoxy, or isopropoxy) optionally substituted with one R²¹, NR⁸R⁹, 3- to 10-membered heterocycloalkyl (e.g., azetidinyl or pyrrolidinyl) optionally substituted with one R²¹, or one pair of R²⁰ on the same atom, taken together with the atom connecting them, independently forms a monocyclic C₃-C₄ carbocyclic ring or a monocyclic 3- to 4-membered heterocyclic ring containing 1 O atom optionally substituted with OS(O)₂Ph; R²¹ is selected from the group consisting of halo (e.g., fluoro), NR⁸R⁹, C₂-C₆ alkynyl (e.g., ethynyl), and C₁-C₆ alkoxy (e.g., methoxy); R⁸ and R⁹ at each occurrence is independently selected from hydrogen, C₁-C₆ alkyl (e.g., methyl or ethyl), COR¹³, and CO₂R¹³; R¹³ is selected from the group consisting of: C₁-C₆ alkyl (e.g., methyl or t-butyl) and C₁-C₆ haloalkyl (e.g., trifluoromethyl); and
the substituted ring B is selected from:

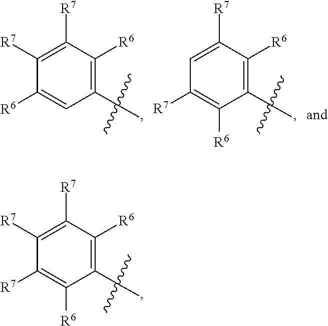

wherein

two pairs, each of one R⁶ and one R⁷, are on adjacent atoms, and each pair of one R⁶ and one R⁷ taken together with the atoms connecting them independently form a C₄-C₇ carbocyclic ring or a 5- to 7-membered heterocyclic ring containing 1 or 2 heteroatoms independently selected from O, N, and S, wherein each carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹; and

the remaining R⁷ that is not taken with a R⁶ on adjacent atoms to form a C₄-C₇ carbocyclic ring or a 5- to 7-membered heterocyclic ring is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy.

### [Combination 4]

In some embodiments, the compound of Formula AA is an unequal mixture (e.g., a non-racemic mixture when the sulfur is the only stereocenter in Formula AA):

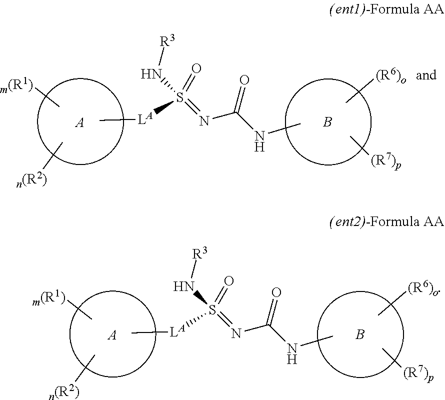

In certain of these embodiments, the mixture is enriched in (ent1)-Formula AA.

In certain of other embodiments, the mixture is enriched in (ent2)-Formula AA.

In certain embodiments, the compound is a compound of (ent1)-Formula AA that is substantially free (e.g., contains less than about 10% of, less than about 5% of, less than about 2% of, less than about 1%, less than about 0.5% of) of (ent2)-Formula AA.

In certain embodiments, the compound is a compound of (ent2)-Formula AA that is substantially free (e.g., contains less than about 10% of, less than about 5% of, less than about 2% of, less than about 1%, less than about 0.5% of) of (ent1)-Formula AA.

In some embodiments, the compound of Formula AA has Formula AA-CN:

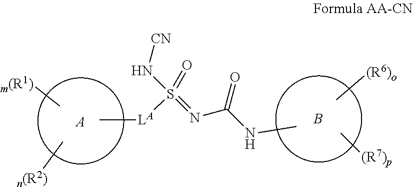

or a pharmaceutically acceptable salt thereof, wherein the variables in Formula AA-CN can be as defined anywhere herein.

In certain of these embodiments, the compound of Formula AA is an unequal mixture (e.g., a non-racemic mixture when the sulfur is the only stereocenter in Formula AA-CN) of:

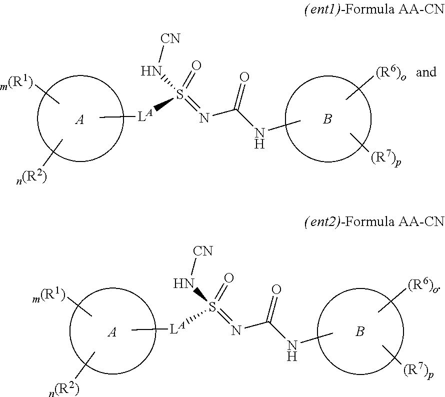

In certain of these embodiments, the mixture is enriched in (ent1)-Formula AA-CN.

In certain of other embodiments, the mixture is enriched in (ent2)-Formula AA-CN.

In certain embodiments, the compound is a compound of (ent1)-Formula AA-CN that is substantially free (e.g., contains less than about 10% of, less than about 5% of, less than about 2% of, less than about 1%, less than about 0.5% of) of (ent2)-Formula AA-CN.

In certain embodiments, the compound is a compound of (ent2)-Formula AA-CN that is substantially free (e.g., contains less than about 10% of, less than about 5% of, less than about 2% of, less than about 1%, less than about 0.5% of) of (ent1)-Formula AA-CN.

The inventors have discovered that in the corresponding N—H (R³═H) compounds, activity primarily resides in only one of the two sulfur enantiomers or epimers; however, surprisingly, when said hydrogen atom is replaced with a cyano group, activity resides primarily in the opposing enantiomer or epimer.

In some embodiments of [combination 4], the optionally substituted ring A is

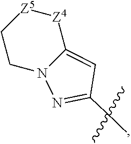

wherein Z⁴ is selected from the group consisting of —CH₂—, —C(O)—, and NH; Z⁵ is selected from the group consisting of O, NH, N—CH₃, and —CH₂—; and

the substituted ring B is selected from:

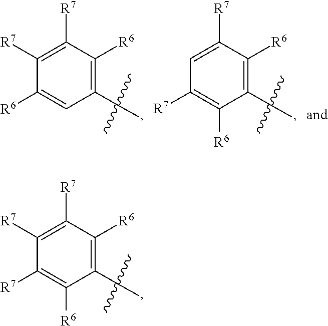

wherein

two pairs, each of one R⁶ and one R⁷, are on adjacent atoms, and each pair of one R⁶ and one R⁷ taken together with the atoms connecting them independently form a C₄-C₇ carbocyclic ring or a 5- to 7-membered heterocyclic ring containing 1 or 2 heteroatoms independently selected from O, N, and S, wherein each carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹; and

the remaining R⁷ that is not taken with a R⁶ on adjacent atoms to form a C₄-C₇ carbocyclic ring or a 5- to 7-membered heterocyclic ring is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy.

In some embodiments of [Combination 3] and [Combination 4], the substituted ring B is

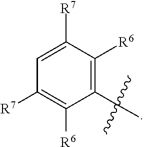

In some embodiments of [Combination 3] and [Combination 4], the substituted ring B is

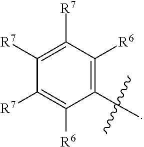

In some embodiments of [Combination 3] and [Combination 4], one pair of R⁶ and R⁷ taken together with the atoms connecting them independently form a C₅ carbocyclic ring, wherein the carbocyclic ring is optionally substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In certain of these embodiments, the other pair of R⁶ and R⁷ taken together with the atoms connecting them independently form a C₅ carbocyclic ring, wherein the carbocyclic ring is optionally substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

As non-limiting examples of the foregoing embodiments, the substituted ring B is selected from:

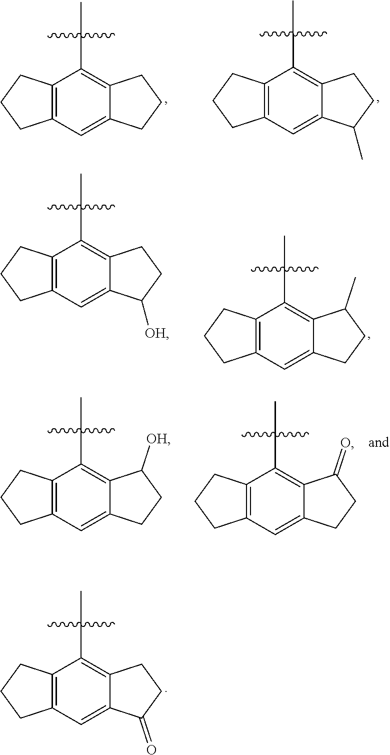

As further non-limiting examples, the substituted ring B is

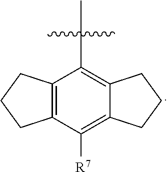

In certain embodiments of [Combination 3] and [Combination 4] (when one pair of R⁶ and R⁷ taken together with the atoms connecting them independently form a C₅ carbocyclic ring, wherein the carbocyclic ring is optionally substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹), the other pair of R⁶ and R⁷ taken together with the atoms connecting them independently form a C₄ carbocyclic ring, wherein the carbocyclic ring is optionally substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹ (e.g., the substituted ring B is:

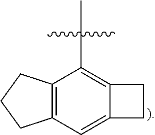 [SMILES: *c1c2c(cc3c1CC3)CCC2]

In some embodiments of [Combination 3] and [Combination 4], one pair of R⁶ and R⁷ taken together with the atoms connecting them independently form a C₄ carbocyclic ring, wherein the carbocyclic ring is optionally substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In certain of these embodiments, the other pair of R⁶ and R⁷ taken together with the atoms connecting them independently form a C₄ carbocyclic ring, wherein the carbocyclic ring is optionally substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹ (e.g., the substituted ring B is:

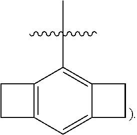 [SMILES: *c1c2c(cc3c1CC3)CC2]

In some embodiments of [Combination 3] and [Combination 4], the substituted ring B is

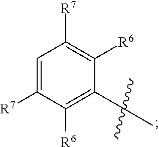

and one pair of R⁶ and R⁷ on adjacent atoms, taken together with the atoms connecting them, independently form one C₄-C₇ (e.g., C₅) carbocyclic ring; and each of the remaining R⁶ and R⁷ is independently selected from C₁-C₆ alkyl, C₁-C₆ haloalkyl, and C₃-C₇ cycloalkyl (e.g., the substituted ring B is

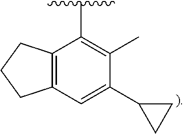 [SMILES: *c1c(C)c(C2CC2)cc2c1CCC2]

In some embodiments [Combination 3] and [Combination 4], the substituted ring B is

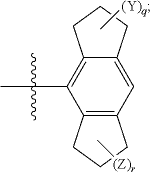

q is 0, 1, or 2; r is 0, 1, or 2; wherein each of Y and Z is independently selected from C₁-C₆ alkyl (e.g., methyl) and hydroxy; or wherein when two Y are attached to the same carbon, the two Y are taken together with the carbon they are attached to to form a cyclopropyl ring; or wherein when two Z are attached to the same carbon, the two Z are taken together with the carbon they are attached to form a cyclopropyl ring.

In some embodiments [Combination 3] and [Combination 4], the substituted ring B is

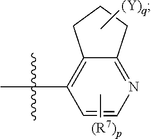

R⁷ is selected from C₁-C₆ alkyl (e.g., methyl, ethyl, or isopropyl), C₆-C₁₀ aryl (e.g., phenyl), and C₃-C₁₀ cycloalkyl (e.g., cyclopropyl); p is 0, 1, or 2; q is 0, 1, or 2; wherein each Y is independently selected from C₁-C₆ alkyl (e.g., methyl) and hydroxy; or when two Y are attached to the same carbon, the two Y are taken together with the carbon they are attached to to form a cyclopropyl ring.

In some embodiments [Combination 3] and [Combination 4], the substituted ring B is

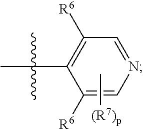

each R⁶ is independently selected from C₁-C₆ alkyl (e.g., isopropyl); each R⁷ is independently selected from halo (e.g., fluoro); p is 0 or 1.

In some embodiments [Combination 3] and [Combination 4], the substituted ring B is

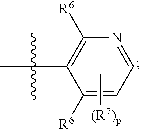

each R⁶ is independently selected from C₁-C₆ alkyl (e.g., isopropyl); each R⁷ is independently selected from halo (e.g., fluoro); p is 0 or 1.

In some embodiments [Combination 3] and [Combination 4], the substituted ring B is

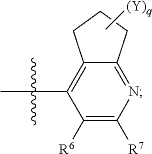

R⁶ is selected from C₁-C₆ alkyl (e.g., methyl, ethyl, or isopropyl) and C₃-C₁₀ cycloalkyl (e.g., cyclopropyl); R⁷ is selected from C₁-C₆ alkyl (e.g., methyl, ethyl, or isopropyl), C₁-C₆ haloalkyl (e.g., trifluoromethyl) and C₃-C₁₀ cycloalkyl (e.g., cyclopropyl or cyclobutyl); or R⁶ and R⁷, taken together with the atoms connecting them, independently form a C₅ carbocyclic ring optionally substituted with one or more C₁-C₆ alkyl (e.g., methyl); q is 0, 1, or 2; each Y is independently selected from C₁-C₆ alkyl (e.g., methyl); or when two Y are attached to the same carbon, the two Y are taken together with the carbon they are attached to to form a cyclopropyl ring.

In some embodiments of [Combination 3] and [Combination 4], L^{A} is a bond.

In some embodiments of [Combination 3] and [Combination 4], L^{A} is CH₂.

### [Combination 5]

In some embodiments, Ring A is

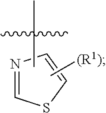

and R¹ is C₁-C₆ alkyl substituted with one or more (e.g., from 1-2) hydroxy (e.g., 2-hydroxy-2-propyl or 1,2-dihydroxy-2-propyl).

In some embodiments, Ring A is

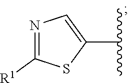

and R¹ is C₁-C₆ alkyl substituted with one or more (e.g., from 1-2) hydroxy (e.g., 2-hydroxy-2-propyl or 1,2-dihydroxy-2-propyl).

In some embodiments, Ring A is

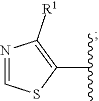

and R¹ is C₁-C₆ alkyl substituted with one or more (e.g., from 1-2) hydroxy (e.g., 2-hydroxy-2-propyl or 1,2-dihydroxy-2-propyl).

In some embodiments, Ring A is

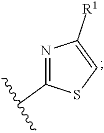

and R¹ is C₁-C₆ alkyl substituted with one or more (e.g., from 1-2) hydroxy (e.g., 2-hydroxy-2-propyl or 1,2-dihydroxy-2-propyl).

In some embodiments, Ring A is

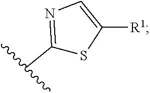

and R¹ is C₁-C₆ alkyl substituted with one or more (e.g., from 1-2) hydroxy (e.g., 2-hydroxy-2-propyl or 1,2-dihydroxy-2-propyl).

In some embodiments, Ring A is

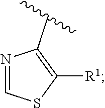

and R¹ is C₁-C₆ alkyl substituted with one or more (e.g., from 1-2) hydroxy (e.g., 2-hydroxy-2-propyl or 1,2-dihydroxy-2-propyl).

In some embodiments, Ring A is

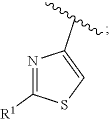

and R¹ is C₁-C₆ alkyl substituted with one or more (e.g., from 1-2) hydroxy (e.g., 2-hydroxy-2-propyl or 1,2-dihydroxy-2-propyl).

In some embodiments, Ring A is

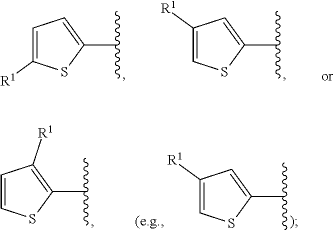

and R¹ is C₁-C₆ alkyl substituted with one or more (e.g., from 1-2) hydroxy (e.g., 2-hydroxy-2-propyl or 1,2-dihydroxy-2-propyl).

In some embodiments, Ring A is

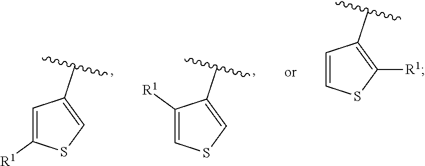

and R¹ is C₁-C₆ alkyl substituted with one or more (e.g., from 1-2) hydroxy (e.g., 2-hydroxy-2-propyl or 1,2-dihydroxy-2-propyl).

In some embodiments of [Combination 6], Ring A is

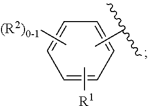

and R¹ is C₁-C₆ alkyl substituted with one or more (e.g., one) NR⁸R⁹ or hydroxy. In certain of these embodiments, R² when present is halo.

In some embodiments, Ring A is

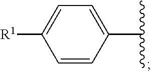

and R¹ is C₁-C₆ alkyl substituted with one or more (e.g., one) NR⁸R⁹.

In some embodiments, Ring A is

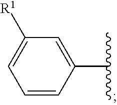

and R¹ is C₁-C₆ alkyl substituted with one or more (e.g., one) NR⁸R⁹.

In some embodiments, Ring A is

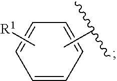

and R¹ is C₁-C₆ alkyl substituted with one or more (e.g., one) hydroxy.

In some embodiments, Ring A is

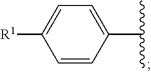

and R¹ is C₁-C₆ alkyl substituted with one or more (e.g., one) hydroxy.

In some embodiments, Ring A is

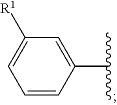

and R¹ is C₁-C₆ alkyl substituted with one or more (e.g., one) hydroxy.

In some embodiments, Ring A is

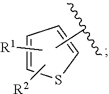

R¹ is C₁-C₆ alkyl substituted with one or more (e.g., from 1-2) hydroxy (e.g., 2-hydroxy-2-propyl or 1,2-dihydroxy-2-propyl); and R² is halo.

In some embodiments, Ring A is

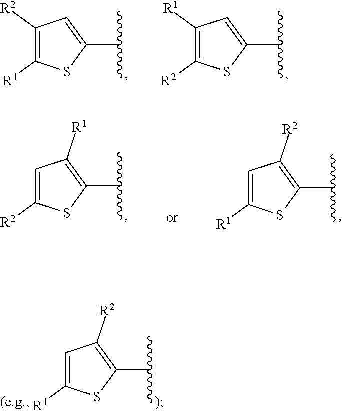

R¹ is C₁-C₆ alkyl substituted with one or more (e.g., from 1-2) hydroxy (e.g., 2-hydroxy-2-propyl or 1,2-dihydroxy-2-propyl); and R² is halo.

In some embodiments, Ring A is

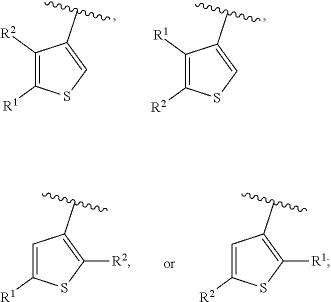

R¹ is C₁-C₆ alkyl substituted with one or more (e.g., from 1-2) hydroxy (e.g., 2-hydroxy-2-propyl or 1,2-dihydroxy-2-propyl); and R² is halo.

In some embodiments, Ring A is

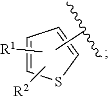

R¹ is C₁-C₆ alkyl substituted with one or more (e.g., one) NR⁸R⁹; and R² is halo.

In some embodiments, Ring A is

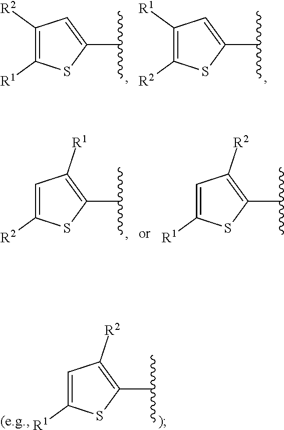

R¹ is C₁-C₆ alkyl substituted with one or more (e.g., one) NR⁸R⁹; and R² is halo.

In some embodiments, Ring A is

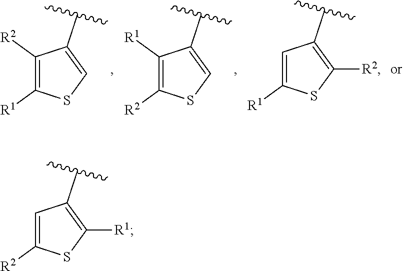

R¹ is C₁-C₆ alkyl substituted with one or more (e.g., one) NR⁸R⁹; and R² is halo.

In some embodiments, Ring A is

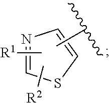

and each of R¹ and R² is C₁-C₆ alkyl substituted with one or more (e.g., from 1-2) hydroxy.

In some embodiments, Ring A is

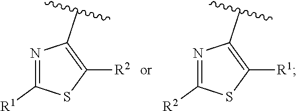

and each of R¹ and R² is C₁-C₆ alkyl substituted with one or more (e.g., from 1-2) hydroxy.

In some embodiments, Ring A is

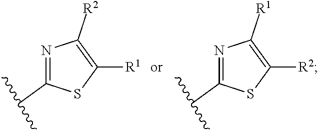

and each of R¹ and R² is C₁-C₆ alkyl substituted with one or more (e.g., from 1-2) hydroxy. In certain of these embodiments, R² is hydroxy methyl.

In some embodiments, Ring A is

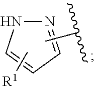

and R¹ is C₁-C₆ alkyl optionally substituted with one or more halo (e.g., ethyl or difluoromethyl).

In some embodiments, Ring A is

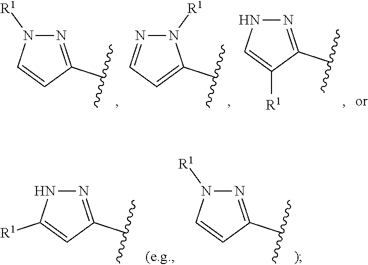

and R¹ is C₁-C₆ alkyl optionally substituted with one or more halo (e.g., ethyl or difluoromethyl).

In some embodiments, Ring A is:

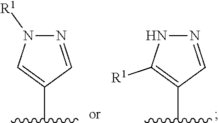

and and R¹ is C₁-C₆ alkyl optionally substituted with one or more halo (e.g., ethyl or difluoromethyl).

In some embodiments, Ring A is

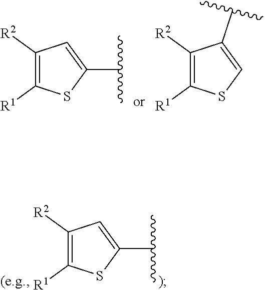

and R¹ and R² taken together with the atoms connecting them, independently form one monocyclic or bicyclic C₄-C₁₂ carbocyclic ring (e.g., C₅ or C₆ carbocyclic ring) or one monocyclic or bicyclic 5- to-12-membered (e.g., 6-membered or 5-membered) heterocyclic ring containing 1-3 (e.g., 1-2, e.g., 1) heteroatoms independently selected from O, N, and S (e.g., N or O), wherein the carbocyclic ring or heterocyclic ring is optionally independently substituted with one or more substituents each independently selected from hydroxy, halo, oxo, C₁-C₆ alkyl (e.g., methyl), C₂-C₆ alkenyl, C₂-C₆ alkynyl, C₁-C₆ alkoxy (e.g., methoxy, ethoxy, isopropoxyl), OC₃-C₁₀ cycloalkyl, NR⁸R⁹, ═NR¹⁰, CN, COOC₁-C₆ alkyl, S(O₂)C₆-C₁₀ aryl, C₆-C₁₀ aryl, 5- to 10-membered heteroaryl, C₃-C₁₀ cycloalkyl, 3- to 10-membered heterocycloalkyl (e.g., azetidinyl or oxetanyl), and CONR⁸R⁹, wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo (e.g., fluoro), C₁-C₆ alkyl, C₂-C₆ alkenyl, C₂-C₆ alkynyl, C₃-C₁₀ cycloalkyl, C₁-C₆ alkoxy, oxo, NR⁸R⁹ (e.g., amino, methylamino, or dimethylamino), ═NR¹⁰, COOC₁-C₆ alkyl, C₆-C₁₀ aryl, and CONR⁸R⁹.

In some embodiments, Ring A is

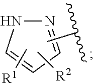

and each of R¹ and R² is C₁-C₆ alkyl substituted with one or more (e.g., from 1-2) hydroxy.

In some embodiments, Ring A is

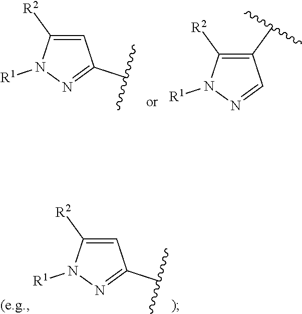

and each of R¹ and R² is C₁-C₆ alkyl substituted with one or more (e.g., from 1-2) hydroxy.

### [Combination 6]

In some embodiments, the compound of Formula AA is an unequal mixture (e.g., a non-racemic mixture when the sulfur is the only stereocenter in Formula AA):

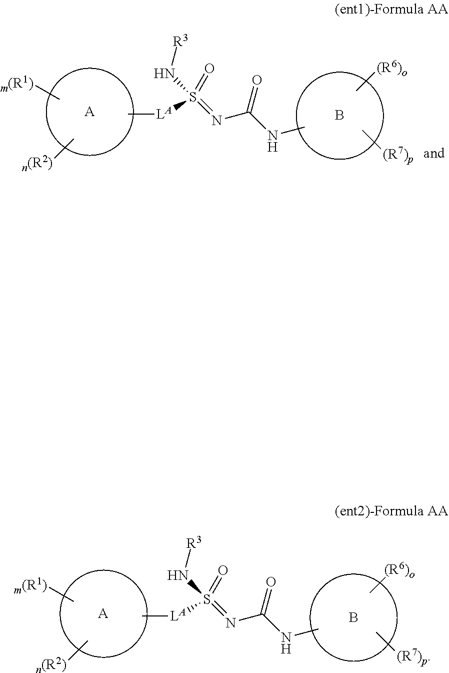

In certain of these embodiments, the mixture is enriched in (ent1)-Formula AA.

In certain of other embodiments, the mixture is enriched in (ent2)-Formula AA.

In certain embodiments, the compound is a compound of (ent1)-Formula AA that is substantially free (e.g., contains less than about 10% of, less than about 5% of, less than about 2% of, less than about 1%, less than about 0.5% of) of (ent2)-Formula AA.

In certain embodiments, the compound is a compound of (ent2)-Formula AA that is substantially free (e.g., contains less than about 10% of, less than about 5% of, less than about 2% of, less than about 1%, less than about 0.5% of) of (ent1)-Formula AA.

In some embodiments, the compound of Formula AA has Formula AA-CN:

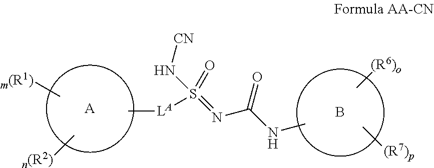

or a pharmaceutically acceptable salt thereof, wherein the variables in Formula AA-CN can be as defined anywhere herein.

In certain of these embodiments, the compound of Formula AA is an unequal mixture (e.g., a non-racemic mixture when the sulfur is the only stereocenter in Formula AA-CN) of:

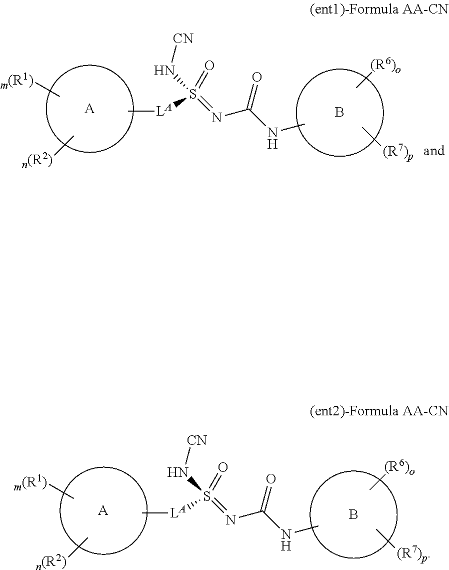

In certain of these embodiments, the mixture is enriched in (ent1)-Formula AA-CN.

In certain of other embodiments, the mixture is enriched in (ent2)-Formula AA-CN.

In certain embodiments, the compound is a compound of (ent1)-Formula AA-CN that is substantially free (e.g., contains less than about 10% of, less than about 5% of, less than about 2% of, less than about 1%, less than about 0.5% of) of (ent2)-Formula AA-CN.

In certain embodiments, the compound is a compound of (ent2)-Formula AA-CN that is substantially free (e.g., contains less than about 10% of, less than about 5% of, less than about 2% of, less than about 1%, less than about 0.5% of) of (ent1)-Formula AA-CN.

The inventors have discovered that in the corresponding N—H (R³═H) compounds, activity primarily resides in only one of the two sulfur enantiomers or epimers; however, surprisingly, when said hydrogen atom is replaced with a cyano group, activity resides primarily in the opposing enantiomer or epimer.

In some embodiments of [Combination 6], Ring A is

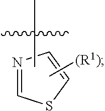

and R¹ is C₁-C₆ alkyl substituted with one or more (e.g., from 1-2) hydroxy (e.g., 2-hydroxy-2-propyl or 1,2-dihydroxy-2-propyl).

In some embodiments of [Combination 6], Ring A is

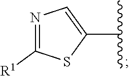

and R¹ is C₁-C₆ alkyl substituted with one or more (e.g., from 1-2) hydroxy (e.g., 2-hydroxy-2-propyl or 1,2-dihydroxy-2-propyl).

In some embodiments of [Combination 6], Ring A is

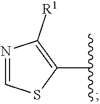

and R¹ is C₁-C₆ alkyl substituted with one or more (e.g., from 1-2) hydroxy (e.g., 2-hydroxy-2-propyl or 1,2-dihydroxy-2-propyl).

In some embodiments of [Combination 6], Ring A is

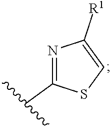

and R¹ is C₁-C₆ alkyl substituted with one or more (e.g., from 1-2) hydroxy (e.g., 2-hydroxy-2-propyl or 1,2-dihydroxy-2-propyl).

In some embodiments of [Combination 6], Ring A is

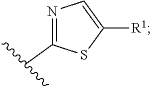

and R¹ is C₁-C₆ alkyl substituted with one or more (e.g., from 1-2) hydroxy (e.g., 2-hydroxy-2-propyl or 1,2-dihydroxy-2-propyl).

In some embodiments of [Combination 6], Ring A is

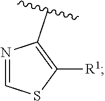

and R¹ is C₁-C₆ alkyl substituted with one or more (e.g., from 1-2) hydroxy (e.g., 2-hydroxy-2-propyl or 1,2-dihydroxy-2-propyl).

In some embodiments of [Combination 6], Ring A is

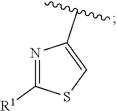

and R¹ is C₁-C₆ alkyl substituted with one or more (e.g., from 1-2) hydroxy (e.g., 2-hydroxy-2-propyl or 1,2-dihydroxy-2-propyl).

In some embodiments of [Combination 6], Ring A is

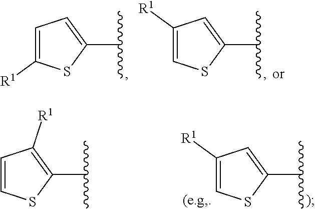

and R¹ is C₁-C₆ alkyl substituted with one or more (e.g., from 1-2) hydroxy (e.g., 2-hydroxy-2-propyl or 1,2-dihydroxy-2-propyl).

In some embodiments of [Combination 6], Ring A is

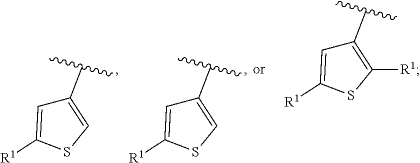

and R¹ is C₁-C₆ alkyl substituted with one or more (e.g., from 1-2) hydroxy (e.g., 2-hydroxy-2-propyl or 1,2-dihydroxy-2-propyl).

In some embodiments of [Combination 6], Ring A is

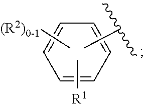

and R¹ is C₁-C₆ alkyl substituted with one or more (e.g., one) NR⁸R⁹ or hydroxy. In certain of these embodiments, R² when present is halo.

In some embodiments of [Combination 6], Ring A is

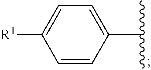

and R¹ is C₁-C₆ alkyl substituted with one or more (e.g., one) NR⁸R⁹.

In some embodiments of [Combination 6], Ring A is

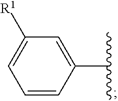

and R¹ is C₁-C₆ alkyl substituted with one or more (e.g., one) NR⁸R⁹.

In some embodiments of [Combination 6], Ring A is

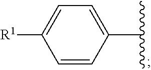

and R¹ is C₁-C₆ alkyl substituted with one or more (e.g., one) hydroxy.

In some embodiments of [Combination 6], Ring A is

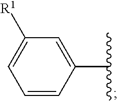

and R¹ is C₁-C₆ alkyl substituted with one or more (e.g., one) hydroxy.

In some embodiments of [Combination 6], Ring A is

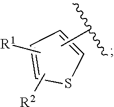

R¹ is C₁-C₆ alkyl substituted with one or more (e.g., from 1-2) hydroxy (e.g., 2-hydroxy-2-propyl or 1,2-dihydroxy-2-propyl); and R² is halo.

In some embodiments of [Combination 6], Ring A is

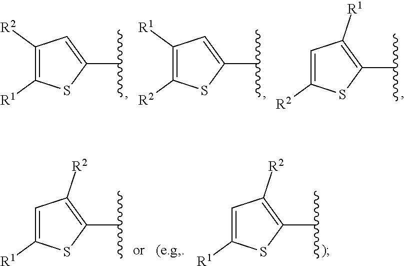

R¹ is C₁-C₆ alkyl substituted with one or more (e.g., from 1-2) hydroxy (e.g., 2-hydroxy-2-propyl or 1,2-dihydroxy-2-propyl); and R² is halo.

In some embodiments of [Combination 6], Ring A is

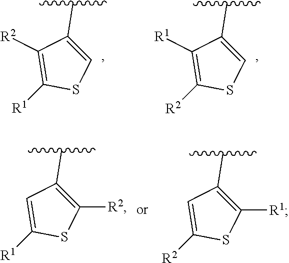

R¹ is C₁-C₆ alkyl substituted with one or more (e.g., from 1-2) hydroxy (e.g., 2-hydroxy-2-propyl or 1,2-dihydroxy-2-propyl); and R² is halo.

In some embodiments of [Combination 6], Ring A is

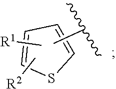

R¹ is C₁-C₆ alkyl substituted with one or more (e.g., one) NR⁸R⁹; and R² is halo.

In some embodiments of [Combination 6], Ring A is

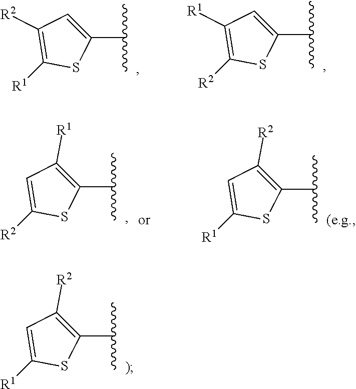

R¹ is C₁-C₆ alkyl substituted with one or more (e.g., one) NR⁸R⁹; and R² is halo.

In some embodiments of [Combination 6], Ring A is

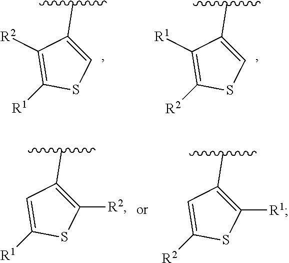

R¹ is C₁-C₆ alkyl substituted with one or more (e.g., one) NR⁸R⁹; and R² is halo.

In some embodiments of [Combination 6], Ring A is

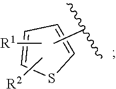

and each of R¹ and R² is C₁-C₆ alkyl substituted with one or more (e.g., from 1-2) hydroxy.

In some embodiments of [Combination 6], Ring A is

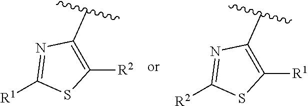

and each of R¹ and R² is C₁-C₆ alkyl substituted with one or more (e.g., from 1-2) hydroxy.

In some embodiments of [Combination 6], Ring A is

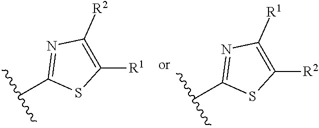

and each of R¹ and R² is C₁-C₆ alkyl substituted with one or more (e.g., from 1-2) hydroxy. In certain of these embodiments, R² is hydroxy methyl.

In some embodiments of [Combination 6], Ring A is

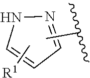

and R¹ is C₁-C₆ alkyl optionally substituted with one or more halo (e.g., ethyl or difluoromethyl).

In some embodiments of [Combination 6], Ring A is

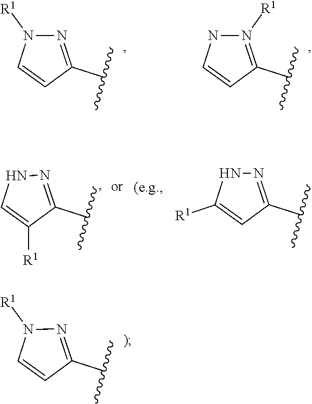

and R¹ is C₁-C₆ alkyl optionally substituted with one or more halo (e.g., ethyl or difluoromethyl).

In some embodiments of [Combination 6], Ring A is:

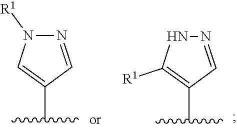

and and R¹ is C₁-C₆ alkyl optionally substituted with one or more halo (e.g., ethyl or difluoromethyl).

In some embodiments of [Combination 6], Ring A is

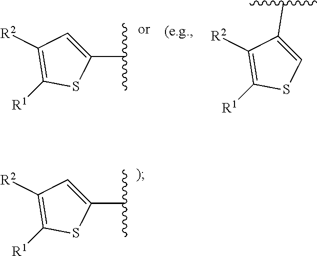

and R¹ and R² taken together with the atoms connecting them, independently form one monocyclic or bicyclic C₄-C₁₂ carbocyclic ring (e.g., C₅ or C₆ carbocyclic ring) or one monocyclic or bicyclic 5- to-12-membered (e.g., 6-membered or 5-membered) heterocyclic ring containing 1-3 (e.g., 1-2, e.g., 1) heteroatoms independently selected from O, N, and S (e.g., N or O), wherein the carbocyclic ring or heterocyclic ring is optionally independently substituted with one or more substituents each independently selected from hydroxy, halo, oxo, C₁-C₆ alkyl (e.g., methyl), C₂-C₆ alkenyl, C₂-C₆ alkynyl, C₁-C₆ alkoxy (e.g., methoxy, ethoxy, isopropoxyl), OC₃-C₁₀ cycloalkyl, NR⁸R⁹, ═NR¹⁰, CN, COOC₁-C₆ alkyl, S(O₂)C₆-C₁₀ aryl, C₆-C₁₀ aryl, 5- to 10-membered heteroaryl, C₃-C₁₀ cycloalkyl, 3- to 10-membered heterocycloalkyl (e.g., azetidinyl or oxetanyl), and CONR⁸R⁹, wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo (e.g., fluoro), C₁-C₆ alkyl, C₂-C₆ alkenyl, C₂-C₆ alkynyl, C₃-C₁₀ cycloalkyl, C₁-C₆ alkoxy, oxo, NR⁸R⁹ (e.g., amino, methylamino, or dimethylamino), ═NR¹⁰, COOC₁-C₆ alkyl, C₆-C₁₀ aryl, and CONR⁸R⁹.

In some embodiments of [Combination 6], Ring A is

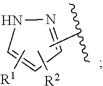

and each of R¹ and R² is C₁-C₆ alkyl substituted with one or more (e.g., from 1-2) hydroxy.

In some embodiments of [Combination 6], Ring A is

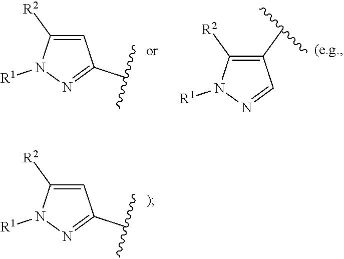

and each of R¹ and R² is C₁-C₆ alkyl substituted with one or more (e.g., from 1-2) hydroxy.

In some embodiments of [Combination 5] and [Combination 6], the substituted ring B is

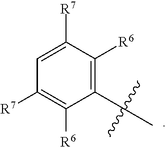

In some embodiments of [Combination 5] and [Combination 6], the substituted ring B is

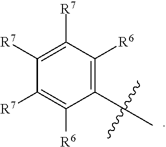

In some embodiments of [Combination 5] and [Combination 6], one pair of R⁶ and R⁷ taken together with the atoms connecting them independently form a C₅ carbocyclic ring, wherein the carbocyclic ring is optionally substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In certain of these embodiments, the other pair of R⁶ and R⁷ taken together with the atoms connecting them independently form a C₅ carbocyclic ring, wherein the carbocyclic ring is optionally substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

As non-limiting examples of the foregoing embodiments, the substituted ring B is selected from:

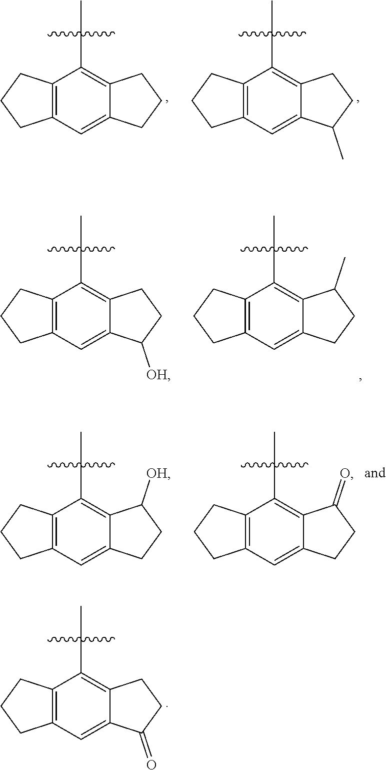

For example,

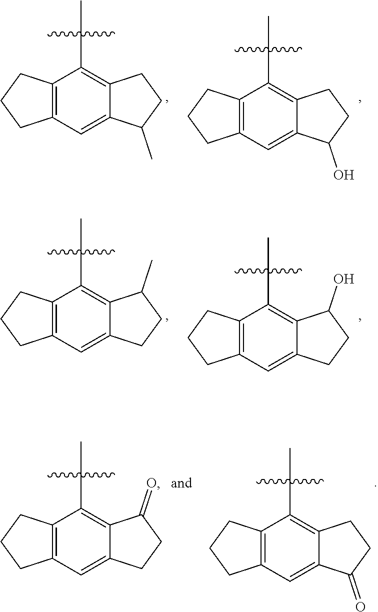

As further non-limiting examples, the substituted ring B is

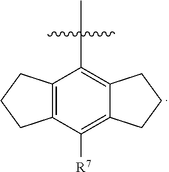

In certain embodiments of [Combination 5] and [Combination 6] (when one pair of R⁶ and R⁷ taken together with the atoms connecting them independently form a C₅ carbocyclic ring, wherein the carbocyclic ring is optionally substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹), the other pair of R⁶ and R⁷ taken together with the atoms connecting them independently form a C₄ carbocyclic ring, wherein the carbocyclic ring is optionally substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹ (e.g., the substituted ring B is:

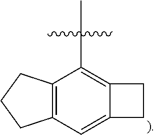 [SMILES: *c1c2c(cc3c1CC3)CCC2]

In some embodiments of [Combination 5] and [Combination 6], one pair of R⁶ and R⁷ taken together with the atoms connecting them independently form a C₄ carbocyclic ring, wherein the carbocyclic ring is optionally substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

In certain of these embodiments, the other pair of R⁶ and R⁷ taken together with the atoms connecting them independently form a C₄ carbocyclic ring, wherein the carbocyclic ring is optionally substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹ (e.g., the substituted ring B is:

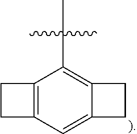 [SMILES: *c1c2c(cc3c1CC3)CC2]

In some embodiments of [Combination 5] and [Combination 6], the substituted ring B is

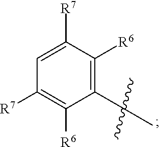

is and one pair of R⁶ and R⁷ on adjacent atoms, taken together with the atoms connecting them, independently form one C₄-C₇ (e.g., C₅) carbocyclic ring; and each of the remaining R⁶ and R⁷ is independently selected from C₁-C₆ alkyl, C₁-C₆ haloalkyl, and C₃-C₇ cycloalkyl (e.g., the substituted ring B is

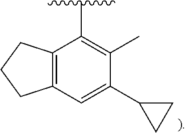 [SMILES: *c1c(C)c(C2CC2)cc2c1CCC2]

In some embodiments [Combination 5] and [Combination 6], the substituted ring B is

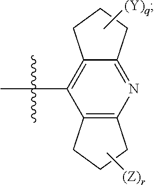

q is 0, 1, or 2; r is 0, 1, or 2; wherein each of Y and Z is independently selected from C₁-C₆ alkyl (e.g., methyl) and hydroxy; or wherein when two Y are attached to the same carbon, the two Y are taken together with the carbon they are attached to to form a cyclopropyl ring; or wherein when two Z are attached to the same carbon, the two Z are taken together with the carbon they are attached to form a cyclopropyl ring.

In some embodiments [Combination 5] and [Combination 6], the substituted ring B is

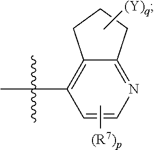

R⁷ is selected from C₁-C₆ alkyl (e.g., methyl, ethyl, or isopropyl), C₆-C₁₀ aryl (e.g., phenyl), and C₃-C₁₀ cycloalkyl (e.g., cyclopropyl); p is 0, 1, or 2; q is 0, 1, or 2; wherein each Y is independently selected from C₁-C₆ alkyl (e.g., methyl) and hydroxy; or when two Y are attached to the same carbon, the two Y are taken together with the carbon they are attached to to form a cyclopropyl ring.

In some embodiments [Combination 5] and [Combination 6], the substituted ring B is

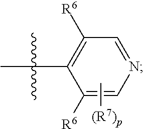

each R⁶ is independently selected from C₁-C₆ alkyl (e.g., isopropyl); each R⁷ is independently selected from halo (e.g., fluoro); p is 0 or 1.

In some embodiments [Combination 5] and [Combination 6], the substituted ring B is

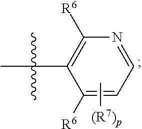

each R⁶ is independently selected from C₁-C₆ alkyl (e.g., isopropyl); each R⁷ is independently selected from halo (e.g., fluoro); p is 0 or 1.

In some embodiments [Combination 5] and [Combination 6], the substituted ring B is

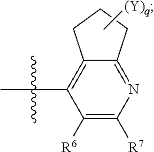

R⁶ is selected from C₁-C₆ alkyl (e.g., methyl, ethyl, or isopropyl) and C₃-C₁₀ cycloalkyl (e.g., cyclopropyl); R⁷ is selected from C₁-C₆ alkyl (e.g., methyl, ethyl, or isopropyl), C₁-C₆ haloalkyl (e.g., trifluoromethyl) and C₃-C₁₀ cycloalkyl (e.g., cyclopropyl or cyclobutyl); or R⁶ and R⁷, taken together with the atoms connecting them, independently form a C₅ carbocyclic ring optionally substituted with one or more C₁-C₆ alkyl (e.g., methyl); q is 0, 1, or 2; each Y is independently selected from C₁-C₆ alkyl (e.g., methyl); or when two Y are attached to the same carbon, the two Y are taken together with the carbon they are attached to to form a cyclopropyl ring.

In some embodiments of [Combination 5] and [Combination 6], L^{A} is a bond.

In some embodiments of [Combination 5] and [Combination 6], L^{A} is CH₂.

In one embodiment, provided herein is a combination of a compound of any preceding embodiment, for use in the treatment or the prevention of a condition mediated by TNF-α, in a patient in need thereof, wherein the compound is administered to said patient at a therapeutically effective amount. Preferably, the subject is resistant to treatment with an anti-TNFα agent. Preferably, the condition is a gut disease or disorder.

In one embodiment, provided herein is a pharmaceutical composition of comprising a compound of any preceding embodiment, and an anti-TNFα agent disclosed herein. Preferably wherein the anti-TNFα agent is Infliximab, Etanercept, Certolizumab pegol, Golimumab or Adalimumab, more preferably wherein the anti-TNFα agent is Adalimumab.

In one embodiment, provided herein is a pharmaceutical combination of a compound of any preceding embodiment, and an anti-TNFα agent Preferably wherein the anti-TNFα agent is Infliximab, Etanercept, Certolizumab pegol, Golimumab or Adalimumab, more preferably wherein the anti-TNFα agent is Adalimumab.

In one embodiment, the present invention relates to an NLRP3 antagonist for use in the treatment or the prevention of a condition mediated by TNF-α, in particular a gut disease or disorder, in a patient in need thereof, wherein the NLRP3 antagonist is administered to said patient at a therapeutically effective amount.

In one embodiment, the present invention relates to an NLRP3 antagonist for use in the treatment or the prevention of a condition, in particular a gut disease or disorder, in a patient in need thereof wherein the NLRP3 antagonist is administered to said patient at a therapeutically effective amount.

In one embodiment, the present invention relates to an NLRP3 antagonist for use in the treatment, stabilization or lessening the severity or progression of gut disease or disorder, in a patient in need thereof wherein the NLRP3 antagonist is administered to said patient at a therapeutically effective amount.

In one embodiment, the present invention relates to an NLRP3 antagonist for use in the slowing, arresting, or reducing the development of a gut disease or disorder, in a patient in need thereof wherein the NLRP3 antagonist is administered to said patient at a therapeutically effective amount.

In one embodiment, the present invention relates to an NLRP3 antagonist for use according to above listed embodiments wherein the NLRP3 antagonist is a gut-targeted NLRP3 antagonist.

In one embodiment, the present invention relates ton NLRP3 antagonist for use according to any of the above embodiments, wherein the gut disease is IBD.

In one embodiment, the present invention relates to an NLRP3 antagonist for use according to any of the above embodiments, wherein the gut disease is UC or CD.

In one embodiment, the present invention relates to a method for the treatment or the prevention of a condition mediated by TNF-a, in particular a gut disease or disorder, in a patient in need thereof, comprising administering to said patient a therapeutically effective amount of a gut-targeted NLRP3 antagonist.

In one embodiment, the present invention relates to a method for the treatment or the prevention of a condition, in particular a gut disease or disorder, in a patient in need thereof, comprising administering to said patient a therapeutically effective amount of a gut-targeted NLRP3 antagonist. In one embodiment, the present invention relates to a method for the treatment, stabilization or lessening the severity or progression of gut disease or disorder, in a patient in need thereof comprising administering to said patient a therapeutically effective amount of a gut-targeted NLRP3 antagonist.

In one embodiment, the present invention relates to a method for slowing, arresting, or reducing the development of a gut disease or disorder, in a patient in need thereof comprising administering to said patient a therapeutically effective amount of a gut-targeted NLRP3 antagonist.

In one embodiment, the present invention relates to a method according to any of the above embodiments, wherein the gut disease is IBD.

In one embodiment, the present invention relates to a method according to any of the above embodiments, wherein the gut disease is UC or CD.

In one embodiment, the present invention relates to a method for the treatment or the prevention of a condition mediated by TNF-a, in particular a gut disease or disorder, in a patient in need thereof, comprising administering to said patient a therapeutically effective amount of a gut-targeted NLRP3 antagonist.

### Additional Features of the Embodiments Herein

In some embodiments the compound of any of the formulae herein is not a compound disclosed in EP 0173498, which is incorporated herein by reference in its entirety.

In some embodiments the compound of any of the formulae herein is not a compound disclosed in U.S. Pat. No. 4,666,506, which is incorporated herein by reference in its entirety.

In some embodiments, the compound of any of the formulae herein is not a compound disclosed in WO 2018225018, which is incorporated herein by reference in its entirety.

In some embodiments, the compound of any one of the formulae herein is not a compound disclosed in IN 201721020305, which is incorporated herein by reference in its entirety.

It is understood that the combination of variables in the formulae herein is such that the compounds are stable.

In some embodiments, provided herein is a compound that is selected from the group consisting of the compounds in Table C1:

TABLE C1

| Com- | |
|---|---|
| pound | |
| # | Structure |
| 101a | 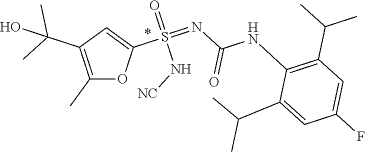 [SMILES: Cc1oc(S(=O)(=NC(=O)Nc2c(C(C)C)cc(F)cc2C(C)C)NC#N)cc1C(C)(C)O] |
| 101b | 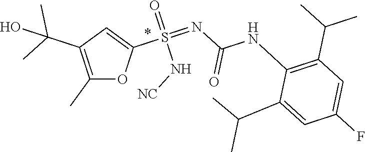 [SMILES: Cc1oc(S(=O)(=NC(=O)Nc2c(C(C)C)cc(F)cc2C(C)C)NC#N)cc1C(C)(C)O] |
| 102a | 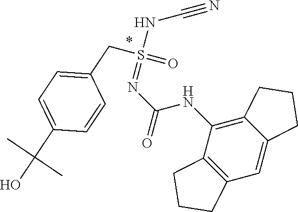 [SMILES: CC(C)(O)c1ccc(CS(=O)(=NC(=O)Nc2c3c(cc4c2CCC4)CCC3)NC#N)cc1] |
| 102b | 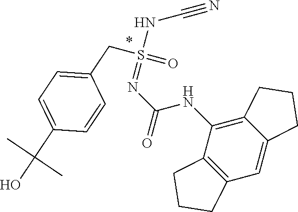 [SMILES: CC(C)(O)c1ccc(CS(=O)(=NC(=O)Nc2c3c(cc4c2CCC4)CCC3)NC#N)cc1] |
| 103a | 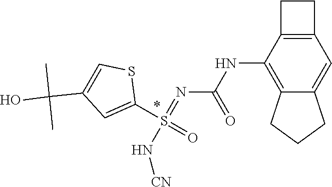 [SMILES: CC(C)(O)c1csc(S(=O)(=NC(=O)Nc2c3c(cc4c2CC4)CCC3)NC#N)c1] |
| 103b | 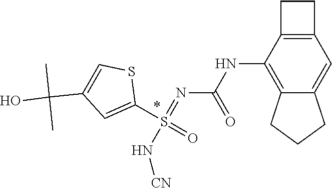 [SMILES: CC(C)(O)c1csc(S(=O)(=NC(=O)Nc2c3c(cc4c2CC4)CCC3)NC#N)c1] |
| 104a | 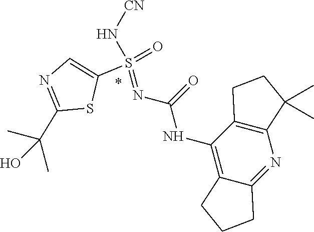 [SMILES: CC(C)(O)c1ncc(S(=O)(=NC(=O)Nc2c3c(nc4c2CCC4(C)C)CCC3)NC#N)s1] |
| 104b | 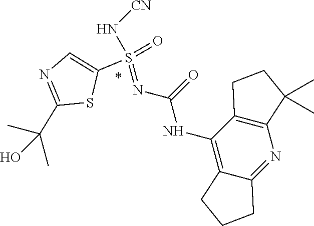 [SMILES: CC(C)(O)c1ncc(S(=O)(=NC(=O)Nc2c3c(nc4c2CCC4(C)C)CCC3)NC#N)s1] |
| 105a | 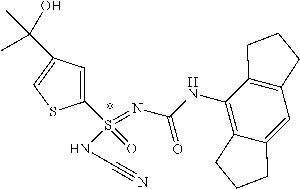 [SMILES: CC(C)(O)c1csc(S(=O)(=NC(=O)Nc2c3c(cc4c2CCC4)CCC3)NC#N)c1] |
| 105b | 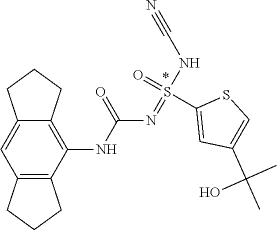 [SMILES: CC(C)(O)c1csc(S(=O)(=NC(=O)Nc2c3c(cc4c2CCC4)CCC3)NC#N)c1] |
| 106 | 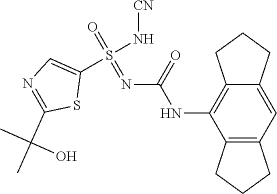 [SMILES: CC(C)(O)c1ncc(S(=O)(=NC(=O)Nc2c3c(cc4c2CCC4)CCC3)NC#N)s1] |
| 108a | 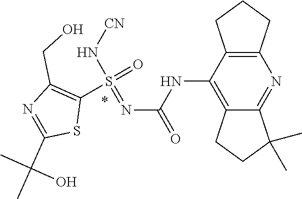 [SMILES: CC(C)(O)c1nc(CO)c(S(=O)(=NC(=O)Nc2c3c(nc4c2CCC4(C)C)CCC3)NC#N)s1] |
| 108b | 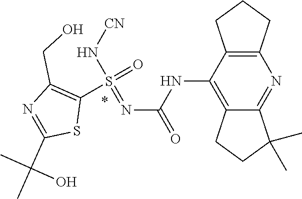 [SMILES: CC(C)(O)c1nc(CO)c(S(=O)(=NC(=O)Nc2c3c(nc4c2CCC4(C)C)CCC3)NC#N)s1] |
| 109a | 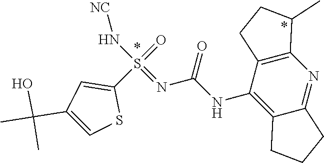 [SMILES: CC1CCc2c1nc1c(c2NC(=O)N=S(=O)(NC#N)c2cc(C(C)(C)O)cs2)CCC1] |
| 109b | 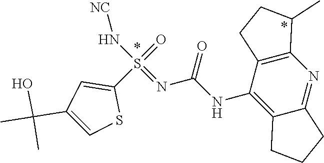 [SMILES: CC1CCc2c1nc1c(c2NC(=O)N=S(=O)(NC#N)c2cc(C(C)(C)O)cs2)CCC1] |
| 109c | 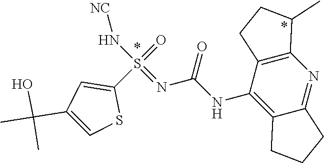 [SMILES: CC1CCc2c1nc1c(c2NC(=O)N=S(=O)(NC#N)c2cc(C(C)(C)O)cs2)CCC1] |
| 109d | 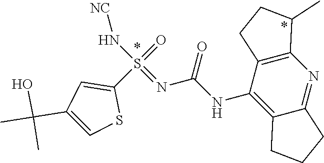 [SMILES: CC1CCc2c1nc1c(c2NC(=O)N=S(=O)(NC#N)c2cc(C(C)(C)O)cs2)CCC1] |
| 110a | 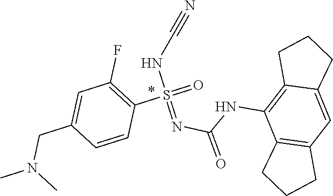 [SMILES: CN(C)Cc1ccc(S(=O)(=NC(=O)Nc2c3c(cc4c2CCC4)CCC3)NC#N)c(F)c1] |
| 110b | 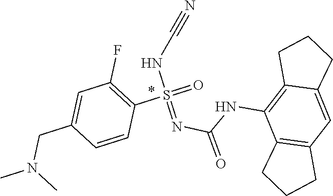 [SMILES: CN(C)Cc1ccc(S(=O)(=NC(=O)Nc2c3c(cc4c2CCC4)CCC3)NC#N)c(F)c1] |

and pharmaceutically acceptable salts thereof.

In some embodiments, a compound of Formula AA is selected from the following:

(R)-N′-cyano-N-((1,2,3,5,6,7-hexahydro-s-indacen-4-yl)carbamoyl)-4-methylbenzene sulfonimidamide;

(R)-N′-cyano-N-((1,2,3,5,6,7-hexahydro-s-indacen-4-yl)carbamoyl)-4-methylbenzene sulfonimidamide;

(R)-N′-cyano-N-((2,6-diisopropylphenyl)carbamoyl)-4-methylbenzenesulfonimidamide;

(R)-N-((2,6-diisopropylphenyl)carbamoyl)-4-methyl-N′-(2,2,2-trifluoroethyl)benzenesulfonimi-damide;

(R)-N-((1,2,3,5,6,7-hexahydro-s-indacen-4-yl)carbamoyl)-4-methyl-N′-(2,2,2-trifluoroethyl)-benzenesulfonimidamide;

(R)-N-((2,6-diisopropylphenyl)carbamoyl)-N′-methoxy-4-methylbenzenesulfonimidamide;

(R)-N-((1,2,3,5,6,7-hexahydro-s-indacen-4-yl)carbamoyl)-N′-isopropyl-4-methylbenzenesulfonimidamide;

(R)-N-((2,6-diisopropylphenyl)carbamoyl)-N′-isopropyl-4-methylbenzenesulfonimidamide;

(R)-N-((1,2,3,5,6,7-hexahydro-s-indacen-4-yl)carbamoyl)-N′-methoxy-4-methylbenzenesulfo-nimidamide;

(R)-N-((1,2,3,5,6,7-hexahydro-s-indacen-4-yl)carbamoyl)-N′,4-dimethylbenzenesulfonimidamide;

(R)-N-((1,2,3,5,6,7-hexahydro-s-indacen-4-yl)carbamoyl)-4-methyl-N′-(1H-pyrazol-5-yl)benzenesulfonimidamide;

(R)-N′-(4-fluorophenyl)-N-((1,2,3,5,6,7-hexahydro-s-indacen-4-yl)carbamoyl)-4-methylbenzenesulfonimidamide;

(R)-N-((1,2,3,5,6,7-hexahydro-s-indacen-4-yl)carbamoyl)-4-methyl-N′-(pyridin-2-yl)benzenesulfonimidamide;

(R)-4-acetyl-N′-cyano-N-((1,2,3,5,6,7-hexahydro-s-indacen-4-yl)carbamoyl)benzenesulfo-nimidamide;

(R)-N′-cyano-N-((1,2,3,5,6,7-hexahydro-s-indacen-4-yl)carbamoyl)-4-(2-hydroxypropan-2-yl)benzenesulfonimidamide;

(R)-N′-cyano-N-((1,2,3,5,6,7-hexahydro-s-indacen-4-yl)carbamoyl)-3-nitrobenzenesulfo-nimidamide;

(R)-N′-cyano-N-((1,2,3,5,6,7-hexaydro-s-indacen-4-yl)carbamoyl)-4-methoxybenzenesulfonimidamide;

(R)-N′-cyano-N-((2,6-diisopropylphenyl)carbamoyl)-4-methoxybenzenesulfonimidamide;

(R)-N′-cyano-4-fluoro-N-((1,2,3,5,6,7-hexahydro-s-indacen-4-yl)carbamoyl)benzenesulfo-nimidamide;

(R)-N′-cyano-N-((1,2,3,5,6,7-hexahydro-s-indacen-4-yl)carbamoyl)benzenesulfonimidamide;

(R)-N-((3-(1,2,3,5,6,7-hexahydro-s-indacen-4-yl)ureido)(oxo)(p-tolyl)-{lambda}6-sulfanylidene)-methanesulfonamide;

(R)-N′-cyano-N-((1,2,3,5,6,7-hexahydro-s-indacen-4-yl)carbamoyl)-6-methoxypyridine-3-sulfonimidamide;

(R)-N′-cyano-3-fluoro-N-((1,2,3,5,6,7-hexahydro-s-indacen-4-yl)carbamoyl)-4-methyl-benzenesulfonimidamide;

(R)-4-chloro-N′-cyano-N-((1,2,3,5,6,7-hexahydro-s-indacen-4-yl)carbamoyl)benzene-sulfonimidamide;

(R)-4-bromo-N′-cyano-N-((1,2,3,5,6,7-hexahydro-s-indacen-4-yl)carbamoyl)benzene-sulfonimidamide;

(R)-4-(benzyloxy)-N′-cyano-N-((1,2,3,5,6,7-hexahydro-s-indacen-4-yl)carbamoyl)benzene-sulfonimidamide;

(R)-N′-cyano-N-((1,2,3,5,6,7-hexahydro-s-indacen-4-yl)carbamoyl)-4-(trifluoromethyl)-benzenesulfonimidamide;

(R)-N′-cyano-N-((1,2,3,5,6,7-hexahydro-s-indacen-4-yl)carbamoyl)pyridine-2-sulfonimidamide;

(R)-Ethyl-((3-(1,2,3,5,6,7-hexahydro-s-indacen-4-yl)ureido)(oxo)(p-tolyl)-{lambda}6-sulfaneylidene)-carbamate;

(R)-N-((3-(1,2,3,5,6,7-hexahydro-s-indacen-4-yl)ureido)(oxo)-(p-tolyl)-{lambda}6-sulfaneylidene)-acetamide;

(R)-N′-carbamoyl-N-((1,2,3,5,6,7-hexahydro-s-indacen-4-yl)carbamoyl)-4-methylbenzene-sulfonimidamide;

(R)-N′-cyano-N-((1,2,3,5,6,7-hexahydro-s-indacen-4-yl)carbamoyl)-3-(2-hydroxypropan-2-yl)benzenesulfonimidamide;

(R)-N′-cyano-N-((1,2,3,5,6,7-hexahydro-s-indacen-4-yl)carbamoyl)-4-(3-hydroxyoxetan-3-yl)benzenesulfonimidamide;

(R)-N′-cyano-N-((1,2,3,5,6,7-hexahydro-s-indacen-4-yl)carbamoyl)-4-(prop-1-en-2-yl)furan-2-sulfonimidamide;

(R)-5-bromo-N′-cyano-N-((1,2,3,5,6,7-hexahydro-s-indacen-4-yl)carbamoyl)thiophene-2-sulfonimidamide;

(R)-N′-cyano-N-((1,2,3,5,6,7-hexahydro-s-indacen-4-yl)carbamoyl)-3-methoxybenzene-sulfonimidamide;

(R)-N′-cyano-N-((1,2,3,5,6,7-hexahydro-s-indacen-4-yl)carbamoyl)-4-(2-hydroxypropan-2-yl)furan-2-sulfonimidamide;

(R)-N′-cyano-N-((1,2,3,5,6,7-hexahydro-s-indacen-4-yl)carbamoyl)-2,3-dihydrobenzo[b][1,4]dioxine-6-sulfonimidamide;

(R)-N′-cyano-N-((1,2,3,5,6,7-hexahydro-s-indacen-4-yl)carbamoyl)-5-(2-hydroxypropan-2-yl)thiophene-2-sulfonimidamide;

(R)-N′-cyano-2-fluoro-N-((1,2,3,5,6,7-hexahydro-s-indacen-4-yl)carbamoyl)-5-methylbenzene-sulfonimidamide;

(R)-N′-cyano-3-(1,1,1,3,3,3-hexafluoro-2-hydroxypropan-2-yl)-N-((1,2,3,5,6,7-hexahydro-s-indacen-4-yl)carbamoyl)benzenesulfonimidamide;

(R)-N′-cyano-3,5-difluoro-N-((1,2,3,5,6,7-hexahydro-s-indacen-4-yl)carbamoyl)benzene-sulfonimidamide;

(R)-N′-cyano-2,4-difluoro-N-((1,2,3,5,6,7-hexahydro-s-indacen-4-yl)carbamoyl)benzene-sulfonimidamide;

(R)-N′-cyano-N-((1,2,3,5,6,7-hexahydro-s-indacen-4-yl)carbamoyl)-3-(trifluoromethoxy)-benzenesulfonimidamide;

(R)-N′-cyano-N-((2,6-diisopropylphenyl)carbamoyl)-3-(2-hydroxypropan-2-yl)benzene-sulfonimidamide;

(R)-N′-cyano-N-((1,2,3,5,6,7-hexahydro-s-indacen-4-yl)carbamoyl)-1-phenylmethanesulfonimidamide;

(R)-N-((3-(1,2,3,5,6,7-hexahydro-s-indacen-4-yl)ureido)(3-(2-hydroxypropan-2-yl)phenyl)(oxo)-{lambda}6-sulfaneylidene)methanesulfonamide;

(R)-N′-cyano-N-((1,2,3,6,7,8-hexahydro-as-indacen-4-yl)carbamoyl)-4-methylbenzenesulfo-nimidamide;

(R)-N′-cyano-N-((1,2,3,5,6,7-hexahydro-s-indacen-4-yl)carbamoyl)thiophene-2-sulfonimidamide;

(R)-N′-cyano-N-((1,2,3,5,6,7-hexahydro-s-indacen-4-yl)carbamoyl)-3,4-dimethoxybenzenesulfonimidamide;

(R)-N′-cyano-N-((1,2,3,5,6,7-hexahydro-s-indacen-4-yl)carbamoyl)-6-methylpyridine-2-sulfonimidamide;

(R)-N′-cyano-N-((1,2,3,5,6,7-hexahydro-s-indacen-4-yl)carbamoyl)-6-methylpyridine-2-sulfonimidamide;

(R)-N′,3-dicyano-N-((1,2,3,5,6,7-hexahydro-s-indacen-4-yl)carbamoyl)benzenesulfonimidamide;

(R)-N′-cyano-N-((1,2,3,5,6,7-hexahydro-s-indacen-4-yl)carbamoyl)pyridine-3-sulfonimidamide;

(R)-N′,4-dicyano-N-((1,2,3,5,6,7-hexahydro-s-indacen-4-yl)carbamoyl)benzene-sulfonimidamide;

(R)-N′-cyano-N-((1,2,3,5,6,7-hexahydro-s-indacen-4-yl)carbamoyl)pyridine-4-sulfonimidamide;

(R)-N′-cyano-3-fluoro-N-((1,2,3,5,6,7-hexahydro-s-indacen-4-yl)carbamoyl)benzene-sulfonimidamide;

(R)-N′-cyano-N-((1,2,3,5,6,7-hexahydro-s-indacen-4-yl)carbamoyl)quinoline-8-sulfonimidamide;

(R)-N′-cyano-N-((1,2,3,5,6,7-hexahydro-s-indacen-4-yl)carbamoyl)-4-morpholinobenzene sulfonimidamide;

(R)-N-(3-(N′-cyano-N-((1,2,3,5,6,7-hexahydro-s-indacen-4-yl)carbamoyl)sulfamidimidoyl)phenyl)acetamide;

(R)-N′-cyano-N-((1,2,3,5,6,7-hexahydro-s-indacen-4-yl)carbamoyl)-3-morpholinobenzene sulfonimidamide;

(R)-N′-cyano-4-fluoro-N-((1,2,3,5,6,7-hexahydro-s-indacen-4-yl)carbamoyl)-3-(2-hydroxypropan-2-yl)benzenesulfonimidamide;

(R)-N′-cyano-2-fluoro-N-((1,2,3,5,6,7-hexahydro-s-indacen-4-yl)carbamoyl)-5-(2-hydroxypropan-2-yl)benzenesulfonimidamide;

(R)-N′-cyano-N-((1,2,3,5,6,7-hexahydro-s-indacen-4-yl)carbamoyl)-5-(2-hydroxypropan-2-yl)pyridine-3-sulfonimidamide;

(R)-N′-cyano-1-(4-cyanophenyl)-N-((1,2,3,5,6,7-hexahydro-s-indacen-4-yl)carbamoyl)-methanesulfonimidamide; and

(R)-N′-cyano-3-(2-hydroxypropan-2-yl)-N-((3-oxo-1,2,3,5,6,7-hexahydro-s-indacen-4-yl)carba-moyl)-benzenesulfonimidamide,

or pharmaceutically acceptable salts of any of the compounds above.

In some embodiments, a compound of Formula AA is selected from the following:

(S)-N′-cyano-N-((1,2,3,5,6,7-hexahydro-s-indacen-4-yl)carbamoyl)-4-methylbenzene sulfonimidamide;

(S)-N′-cyano-N-((1,2,3,5,6,7-hexahydro-s-indacen-4-yl)carbamoyl)-4-methylbenzene sulfonimidamide;

(S)-N′-cyano-N-((2,6-diisopropylphenyl)carbamoyl)-4-methylbenzenesulfonimidamide;

(S)-N-((2,6-diisopropylphenyl)carbamoyl)-4-methyl-N′-(2,2,2-trifluoroethyl)benzenesulfonimi-damide;

(S)-N-((1,2,3,5,6,7-hexahydro-s-indacen-4-yl)carbamoyl)-4-methyl-N′-(2,2,2-trifluoroethyl)-benzenesulfonimidamide;

(S)-N-((2,6-diisopropylphenyl)carbamoyl)-N′-methoxy-4-methylbenzenesulfonimidamide;

(S)-N-((1,2,3,5,6,7-hexahydro-s-indacen-4-yl)carbamoyl)-N′-isopropyl-4-methylbenzenesulfonimidamide;

(S)-N-((2,6-diisopropylphenyl)carbamoyl)-N′-isopropyl-4-methylbenzenesulfonimidamide;

(S)-N-((1,2,3,5,6,7-hexahydro-s-indacen-4-yl)carbamoyl)-N′-methoxy-4-methylbenzenesulfo-nimidamide;

(S)-N-((1,2,3,5,6,7-hexahydro-s-indacen-4-yl)carbamoyl)-N′,4-dimethylbenzenesulfonimidamide;

(S)-N-((1,2,3,5,6,7-hexahydro-s-indacen-4-yl)carbamoyl)-4-methyl-N′-(1H-pyrazol-5-yl)benzenesulfonimidamide;

(S)-N′-(4-fluorophenyl)-N-((1,2,3,5,6,7-hexahydro-s-indacen-4-yl)carbamoyl)-4-methylbenzenesulfonimidamide;

(S)-N-((1,2,3,5,6,7-hexahydro-s-indacen-4-yl)carbamoyl)-4-methyl-N′-(pyridin-2-yl)benzenesulfonimidamide;

(S)-4-acetyl-N′-cyano-N-((1,2,3,5,6,7-hexahydro-s-indacen-4-yl)carbamoyl)benzenesulfo-nimidamide;

(S)-N′-cyano-N-((1,2,3,5,6,7-hexahydro-s-indacen-4-yl)carbamoyl)-4-(2-hydroxypropan-2-yl)benzenesulfonimidamide;

(S)-N′-cyano-N-((1,2,3,5,6,7-hexahydro-s-indacen-4-yl)carbamoyl)-3-nitrobenzenesulfo-nimidamide;

(S)-N′-cyano-N-((1,2,3,5,6,7-hexaydro-s-indacen-4-yl)carbamoyl)-4-methoxybenzenesulfonimidamide;

(S)-N′-cyano-N-((2,6-diisopropylphenyl)carbamoyl)-4-methoxybenzenesulfonimidamide;

(S)-N′-cyano-4-fluoro-N-((1,2,3,5,6,7-hexahydro-s-indacen-4-yl)carbamoyl)benzenesulfo-nimidamide;

(S)-N′-cyano-N-((1,2,3,5,6,7-hexahydro-s-indacen-4-yl)carbamoyl)benzenesulfonimidamide;

(S)-N-((3-(1,2,3,5,6,7-hexahydro-s-indacen-4-yl)ureido)(oxo)(p-tolyl)-{lambda}6-sulfanylidene)-methanesulfonamide;

(S)-N′-cyano-N-((1,2,3,5,6,7-hexahydro-s-indacen-4-yl)carbamoyl)-6-methoxypyridine-3-sulfonimidamide;

(S)-N′-cyano-3-fluoro-N-((1,2,3,5,6,7-hexahydro-s-indacen-4-yl)carbamoyl)-4-methyl-benzenesulfonimidamide;

(S)-4-chloro-N′-cyano-N-((1,2,3,5,6,7-hexahydro-s-indacen-4-yl)carbamoyl)benzene-sulfonimidamide;

(S)-4-bromo-N′-cyano-N-((1,2,3,5,6,7-hexahydro-s-indacen-4-yl)carbamoyl)benzene-sulfonimidamide;

(S)-4-(benzyloxy)-N′-cyano-N-((1,2,3,5,6,7-hexahydro-s-indacen-4-yl)carbamoyl)benzene-sulfonimidamide;

(S)-N′-cyano-N-((1,2,3,5,6,7-hexahydro-s-indacen-4-yl)carbamoyl)-4-(trifluoromethyl)-benzenesulfonimidamide;

(S)-N′-cyano-N-((1,2,3,5,6,7-hexahydro-s-indacen-4-yl)carbamoyl)pyridine-2-sulfonimidamide;

(S)-Ethyl4(3-(1,2,3,5,6,7-hexahydro-s-indacen-4-yl)ureido)(oxo)(p-tolyl)-{lambda}6-sulfaneylidene)-carbamate;

(S)-N-((3-(1,2,3,5,6,7-hexahydro-s-indacen-4-yl)ureido)(oxo)-(p-tolyl)-{lambda}6-sulfaneylidene)-acetamide;

(S)-N′-carbamoyl-N-((1,2,3,5,6,7-hexahydro-s-indacen-4-yl)carbamoyl)-4-methylbenzene-sulfonimidamide;

(S)-N′-cyano-N-((1,2,3,5,6,7-hexahydro-s-indacen-4-yl)carbamoyl)-3-(2-hydroxypropan-2-yl)benzenesulfonimidamide;

(S)-N′-cyano-N-((1,2,3,5,6,7-hexahydro-s-indacen-4-yl)carbamoyl)-4-(3-hydroxyoxetan-3-yl)benzenesulfonimidamide;

(S)-N′-cyano-N-((1,2,3,5,6,7-hexahydro-s-indacen-4-yl)carbamoyl)-4-(prop-1-en-2-yl)furan-2-sulfonimidamide;

(S)-5-bromo-N′-cyano-N-((1,2,3,5,6,7-hexahydro-s-indacen-4-yl)carbamoyl)thiophene-2-sulfonimidamide;

(S)-N′-cyano-N-((1,2,3,5,6,7-hexahydro-s-indacen-4-yl)carbamoyl)-3-methoxybenzene-sulfonimidamide;

(S)-N′-cyano-N-((1,2,3,5,6,7-hexahydro-s-indacen-4-yl)carbamoyl)-4-(2-hydroxypropan-2-yl)furan-2-sulfonimidamide;

(S)-N′-cyano-N-((1,2,3,5,6,7-hexahydro-s-indacen-4-yl)carbamoyl)-2,3-dihydrobenzo[b][1,4]dioxine-6-sulfonimidamide;

(S)-N′-cyano-N-((1,2,3,5,6,7-hexahydro-s-indacen-4-yl)carbamoyl)-5-(2-hydroxypropan-2-yl)thiophene-2-sulfonimidamide;

(S)-N′-cyano-2-fluoro-N-((1,2,3,5,6,7-hexahydro-s-indacen-4-yl)carbamoyl)-5-methylbenzene-sulfonimidamide;

(S)-N′-cyano-3-(1,1,1,3,3,3-hexafluoro-2-hydroxypropan-2-yl)-N-((1,2,3,5,6,7-hexahydro-s-indacen-4-yl)carbamoyl)benzenesulfonimidamide;

(S)-N′-cyano-3,5-difluoro-N-((1,2,3,5,6,7-hexahydro-s-indacen-4-yl)carbamoyl)benzene-sulfonimidamide;

(S)-N′-cyano-2,4-difluoro-N-((1,2,3,5,6,7-hexahydro-s-indacen-4-yl)carbamoyl)benzene-sulfonimidamide;

(S)-N′-cyano-N-((1,2,3,5,6,7-hexahydro-s-indacen-4-yl)carbamoyl)-3-(trifluoromethoxy)-benzenesulfonimidamide;

(S)-N′-cyano-N-((2,6-diisopropylphenyl)carbamoyl)-3-(2-hydroxypropan-2-yl)benzene-sulfonimidamide;

(S)-N′-cyano-N-((1,2,3,5,6,7-hexahydro-s-indacen-4-yl)carbamoyl)-1-phenylmethanesulfonimidamide;

(S)-N-((3-(1,2,3,5,6,7-hexahydro-s-indacen-4-yl)ureido)(3-(2-hydroxypropan-2-yl)phenyl)(oxo)-{lambda}6-sulfaneylidene)methanesulfonamide;

(S)-N′-cyano-N-((1,2,3,6,7,8-hexahydro-as-indacen-4-yl)carbamoyl)-4-methylbenzenesulfo-nimidamide;

(S)-N′-cyano-N-((1,2,3,5,6,7-hexahydro-s-indacen-4-yl)carbamoyl)thiophene-2-sulfonimidamide;

(S)-N′-cyano-N-((1,2,3,5,6,7-hexahydro-s-indacen-4-yl)carbamoyl)-3,4-dimethoxybenzenesulfonimidamide;

(S)-N′-cyano-N-((1,2,3,5,6,7-hexahydro-s-indacen-4-yl)carbamoyl)-6-methylpyridine-2-sulfonimidamide;

(S)-N′-cyano-N-((1,2,3,5,6,7-hexahydro-s-indacen-4-yl)carbamoyl)-6-methylpyridine-2-sulfonimidamide;

(S)-N′,3-dicyano-N-((1,2,3,5,6,7-hexahydro-s-indacen-4-yl)carbamoyl)benzenesulfonimidamide;

(S)-N′-cyano-N-((1,2,3,5,6,7-hexahydro-s-indacen-4-yl)carbamoyl)pyridine-3-sulfonimidamide;

(S)-N′,4-dicyano-N-((1,2,3,5,6,7-hexahydro-s-indacen-4-yl)carbamoyl)benzene-sulfonimidamide;

(S)-N′-cyano-N-((1,2,3,5,6,7-hexahydro-s-indacen-4-yl)carbamoyl)pyridine-4-sulfonimidamide; (S)-N′-cyano-3-fluoro-N-((1,2,3,5,6,7-hexahydro-s-indacen-4-yl)carbamoyl)benzene-sulfonimidamide;

(S)-N′-cyano-N-((1,2,3,5,6,7-hexahydro-s-indacen-4-yl)carbamoyl)quinoline-8-sulfonimidamide;

(S)-N′-cyano-N-((1,2,3,5,6,7-hexahydro-s-indacen-4-yl)carbamoyl)-4-morpholinobenzene sulfonimidamide;

(S)-N-(3-(N′-cyano-N-((1,2,3,5,6,7-hexahydro-s-indacen-4-yl)carbamoyl)sulfamidimidoyl)phenyl)acetamide;

(S)-N′-cyano-N-((1,2,3,5,6,7-hexahydro-s-indacen-4-yl)carbamoyl)-3-morpholinobenzene sulfonimidamide;

(S)-N′-cyano-4-fluoro-N-((1,2,3,5,6,7-hexahydro-s-indacen-4-yl)carbamoyl)-3-(2-hydroxypropan-2-yl)benzenesulfonimidamide;

(S)-N′-cyano-2-fluoro-N-((1,2,3,5,6,7-hexahydro-s-indacen-4-yl)carbamoyl)-5-(2-hydroxypropan-2-yl)benzenesulfonimidamide;

(S)-N′-cyano-N-((1,2,3,5,6,7-hexahydro-s-indacen-4-yl)carbamoyl)-5-(2-hydroxypropan-2-yl)pyridine-3-sulfonimidamide;

(S)-N′-cyano-1-(4-cyanophenyl)-N-((1,2,3,5,6,7-hexahydro-s-indacen-4-yl)carbamoyl)-methanesulfonimidamide; and

(S)-N′-cyano-3-(2-hydroxypropan-2-yl)-N-((3-oxo-1,2,3,5,6,7-hexahydro-s-indacen-4-yl)carba-moyl)-benzenesulfonimidamide,

or pharmaceutically acceptable salts of any of the compounds above.

In some embodiments, provided herein is a compound that is selected from the group consisting of the compounds in the following table:

and pharmaceutically acceptable salts thereof.

Pharmaceutical Compositions and Administration

General

In some embodiments, a chemical entity (e.g., a compound that modulates (e.g., antagonizes) NLRP3, or a pharmaceutically acceptable salt, and/or hydrate, and/or cocrystal, and/or drug combination thereof) is administered as a pharmaceutical composition that includes the chemical entity and one or more pharmaceutically acceptable excipients, and optionally one or more additional therapeutic agents as described herein.

In some embodiments, the chemical entities can be administered in combination with one or more conventional pharmaceutical excipients. Pharmaceutically acceptable excipients include, but are not limited to, ion exchangers, alumina, aluminum stearate, lecithin, self-emulsifying drug delivery systems (SEDDS) such as d-a-tocopherol polyethylene glycol 1000 succinate, surfactants used in pharmaceutical dosage forms such as Tweens, poloxamers or other similar polymeric delivery matrices, serum proteins, such as human serum albumin, buffer substances such as phosphates, tris, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium-chloride, zinc salts, colloidal silica, magnesium tri silicate, polyvinyl pyrrolidone, cellulose-based substances, polyethylene glycol, sodium carboxymethyl cellulose, polyacrylates, waxes, polyethylene-polyoxypropylene-block polymers, and wool fat. Cyclodextrins such as α-, β, and γ-cyclodextrin, or chemically modified derivatives such as hydroxyalkylcyclodextrins, including 2- and 3-hydroxypropyl-β-cyclodextrins, or other solubilized derivatives can also be used to enhance delivery of compounds described herein. Dosage forms or compositions containing a chemical entity as described herein in the range of 0.005% to 100% with the balance made up from non-toxic excipient may be prepared. The contemplated compositions may contain 0.001%-100% of a chemical entity provided herein, in one embodiment 0.1-95%, in another embodiment 75-85%, in a further embodiment 20-80%. Actual methods of preparing such dosage forms are known, or will be apparent, to those skilled in this art; for example, see Remington: The Science and Practice of Pharmacy, 22^{nd} Edition (Pharmaceutical Press, London, UK. 2012).

Routes of Administration and Composition Components

In some embodiments, the chemical entities described herein or a pharmaceutical composition thereof can be administered to subject in need thereof by any accepted route of administration. Acceptable routes of administration include, but are not limited to, buccal, cutaneous, endocervical, endosinusial, endotracheal, enteral, epidural, interstitial, intra-abdominal, intra-arterial, intrabronchial, intrabursal, intracerebral, intracisternal, intracoronary, intradermal, intraductal, intraduodenal, intradural, intraepidermal, intraesophageal, intragastric, intragingival, intraileal, intralymphatic, intramedullary, intrameningeal, intramuscular, intraovarian, intraperitoneal, intraprostatic, intrapulmonary, intrasinal, intraspinal, intrasynovial, intratesticular, intrathecal, intratubular, intratumoral, intrauterine, intravascular, intravenous, nasal, nasogastric, oral, parenteral, percutaneous, peridural, rectal, respiratory (inhalation), subcutaneous, sublingual, submucosal, topical, transdermal, transmucosal, transtracheal, ureteral, urethral and vaginal. In certain embodiments, a preferred route of administration is parenteral (e.g., intratumoral).

Compositions can be formulated for parenteral administration, e.g., formulated for injection via the intravenous, intramuscular, sub-cutaneous, or even intraperitoneal routes. Typically, such compositions can be prepared as injectables, either as liquid solutions or suspensions; solid forms suitable for use to prepare solutions or suspensions upon the addition of a liquid prior to injection can also be prepared; and the preparations can also be emulsified. The preparation of such formulations will be known to those of skill in the art in light of the present disclosure.

The pharmaceutical forms suitable for injectable use include sterile aqueous solutions or dispersions; formulations including sesame oil, peanut oil, or aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

The carrier also can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques, which yield a powder of the active ingredient, plus any additional desired ingredient from a previously sterile-filtered solution thereof

Intratumoral injections are discussed, e.g., in Lammers, et al., “Effect of Intratumoral Injection on the Biodistribution and the Therapeutic Potential of HPMA Copolymer-Based Drug Delivery Systems” Neoplasia. 2006, 10, 788-795.

In certain embodiments, the chemical entities described herein or a pharmaceutical composition thereof are suitable for local, topical administration to the digestive or GI tract, e.g., rectal administration. Rectal compositions include, without limitation, enemas, rectal gels, rectal foams, rectal aerosols, suppositories, jelly suppositories, and enemas (e.g., retention enemas).

Pharmacologically acceptable excipients usable in the rectal composition as a gel, cream, enema, or rectal suppository, include, without limitation, any one or more of cocoa butter glycerides, synthetic polymers such as polyvinylpyrrolidone, PEG (like PEG ointments), glycerine, glycerinated gelatin, hydrogenated vegetable oils, poloxamers, mixtures of polyethylene glycols of various molecular weights and fatty acid esters of polyethylene glycol Vaseline, anhydrous lanolin, shark liver oil, sodium saccharinate, menthol, sweet almond oil, sorbitol, sodium benzoate, anoxid SBN, vanilla essential oil, aerosol, parabens in phenoxyethanol, sodium methyl p-oxybenzoate, sodium propyl p-oxybenzoate, diethylamine, carbomers, carbopol, methyloxybenzoate, macrogol cetostearyl ether, cocoyl caprylocaprate, isopropyl alcohol, propylene glycol, liquid paraffin, xanthan gum, carboxy-metabisulfite, sodium edetate, sodium benzoate, potassium metabisulfite, grapefruit seed extract, methyl sulfonyl methane (MSM) , lactic acid, glycine, vitamins, such as vitamin A and E and potassium acetate.

In certain embodiments, suppositories can be prepared by mixing the chemical entities described herein with suitable non-irritating excipients or carriers such as cocoa butter, polyethylene glycol or a suppository wax which are solid at ambient temperature but liquid at body temperature and therefore melt in the rectum and release the active compound. In other embodiments, compositions for rectal administration are in the form of an enema.

In other embodiments, the compounds described herein or a pharmaceutical composition thereof are suitable for local delivery to the digestive or GI tract by way of oral administration (e.g., solid or liquid dosage forms.).

Solid dosage forms for oral administration include capsules, tablets, pills, powders, and granules. In such solid dosage forms, the chemical entity is mixed with one or more pharmaceutically acceptable excipients, such as sodium citrate or dicalcium phosphate and/or: a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents. Solid compositions of a similar type may also be employed as fillers in soft and hard-filled gelatin capsules using such excipients as lactose or milk sugar as well as high molecular weight polyethylene glycols and the like.

In one embodiment, the compositions will take the form of a unit dosage form such as a pill or tablet and thus the composition may contain, along with a chemical entity provided herein, a diluent such as lactose, sucrose, dicalcium phosphate, or the like; a lubricant such as magnesium stearate or the like; and a binder such as starch, gum acacia, polyvinylpyrrolidine, gelatin, cellulose, cellulose derivatives or the like. In another solid dosage form, a powder, marume, solution or suspension (e.g., in propylene carbonate, vegetable oils, PEG's, poloxamer 124 or triglycerides) is encapsulated in a capsule (gelatin or cellulose base capsule). Unit dosage forms in which one or more chemical entities provided herein or additional active agents are physically separated are also contemplated; e.g., capsules with granules (or tablets in a capsule) of each drug; two-layer tablets; two-compartment gel caps, etc. Enteric coated or delayed release oral dosage forms are also contemplated.

Other physiologically acceptable compounds include wetting agents, emulsifying agents, dispersing agents or preservatives that are particularly useful for preventing the growth or action of microorganisms. Various preservatives are well known and include, for example, phenol and ascorbic acid.

In certain embodiments the excipients are sterile and generally free of undesirable matter. These compositions can be sterilized by conventional, well-known sterilization techniques. For various oral dosage form excipients such as tablets and capsules sterility is not required. The USP/NF standard is usually sufficient.

In certain embodiments, solid oral dosage forms can further include one or more components that chemically and/or structurally predispose the composition for delivery of the chemical entity to the stomach or the lower GI; e.g., the ascending colon and/or transverse colon and/or distal colon and/or small bowel. Exemplary formulation techniques are described in, e.g., Filipski, K. J., et al., Current Topics in Medicinal Chemistry, 2013, 13, 776-802, which is incorporated herein by reference in its entirety.

Examples include upper-GI targeting techniques, e.g., Accordion Pill (Intec Pharma), floating capsules, and materials capable of adhering to mucosal walls.

Other examples include lower-GI targeting techniques. For targeting various regions in the intestinal tract, several enteric/pH-responsive coatings and excipients are available. These materials are typically polymers that are designed to dissolve or erode at specific pH ranges, selected based upon the GI region of desired drug release. These materials also function to protect acid labile drugs from gastric fluid or limit exposure in cases where the active ingredient may be irritating to the upper GI (e.g., hydroxypropyl methylcellulose phthalate series, Coateric (polyvinyl acetate phthalate), cellulose acetate phthalate, hydroxypropyl methylcellulose acetate succinate, Eudragit series (methacrylic acidmethyl methacrylate copolymers), and Marcoat). Other techniques include dosage forms that respond to local flora in the GI tract, Pressure-controlled colon delivery capsule, and Pulsincap.

Ocular compositions can include, without limitation, one or more of any of the following: viscogens (e.g., Carboxymethylcellulose, Glycerin, Polyvinylpyrrolidone, Polyethylene glycol); Stabilizers (e.g., Pluronic (triblock copolymers), Cyclodextrins); Preservatives (e.g., Benzalkonium chloride, ETDA, SofZia (boric acid, propylene glycol, sorbitol, and zinc chloride; Alcon Laboratories, Inc.), Purite (stabilized oxychloro complex; Allergan, Inc.)).

Topical compositions can include ointments and creams. Ointments are semisolid preparations that are typically based on petrolatum or other petroleum derivatives. Creams containing the selected active agent are typically viscous liquid or semisolid emulsions, often either oil-in-water or water-in-oil. Cream bases are typically water-washable, and contain an oil phase, an emulsifier and an aqueous phase. The oil phase, also sometimes called the “internal” phase, is generally comprised of petrolatum and a fatty alcohol such as cetyl or stearyl alcohol; the aqueous phase usually, although not necessarily, exceeds the oil phase in volume, and generally contains a humectant. The emulsifier in a cream formulation is generally a nonionic, anionic, cationic or amphoteric surfactant. As with other carriers or vehicles, an ointment base should be inert, stable, nonirritating and non-sensitizing.

In any of the foregoing embodiments, pharmaceutical compositions described herein can include one or more one or more of the following: lipids, interbilayer crosslinked multilamellar vesicles, biodegradeable poly(D,L-lactic-co-glycolic acid) [PLGA]-based or poly anhydride-based nanoparticles or microparticles, and nanoporous particle-supported lipid bilayers.

Enema Formulations

In some embodiments, enema formulations containing the chemical entities described herein are provided in “ready-to-use” form.

In some embodiments, enema formulations containing the chemical entities described herein are provided in one or more kits or packs. In certain embodiments, the kit or pack includes two or more separately contained/packaged components, e.g. two components, which when mixed together, provide the desired formulation (e.g., as a suspension). In certain of these embodiments, the two component system includes a first component and a second component, in which: (i) the first component (e.g., contained in a sachet) includes the chemical entity (as described anywhere herein) and optionally one or more pharmaceutically acceptable excipients (e.g., together formulated as a solid preparation, e.g., together formulated as a wet granulated solid preparation); and (ii) the second component (e.g., contained in a vial or bottle) includes one or more liquids and optionally one or more other pharmaceutically acceptable excipients together forming a liquid carrier. Prior to use (e.g., immediately prior to use), the contents of (i) and (ii) are combined to form the desired enema formulation, e.g., as a suspension. In other embodiments, each of component (i) and (ii) is provided in its own separate kit or pack.

In some embodiments, each of the one or more liquids is water, or a physiologically acceptable solvent, or a mixture of water and one or more physiologically acceptable solvents. Typical such solvents include, without limitation, glycerol, ethylene glycol, propylene glycol, polyethylene glycol and polypropylene glycol. In certain embodiments, each of the one or more liquids is water. In other embodiments, each of the one or more liquids is an oil, e.g. natural and/or synthetic oils that are commonly used in pharmaceutical preparations.

Further pharmaceutical excipients and carriers that may be used in the pharmaceutical products herein described are listed in various handbooks (e.g. D. E. Bugay and W. P. Findlay (Eds) Pharmaceutical excipients (Marcel Dekker, New York, 1999), E-M Hoepfner, A. Reng and P. C. Schmidt (Eds) Fiedler Encyclopedia of Excipients for Pharmaceuticals, Cosmetics and Related Areas (Edition Cantor, Munich, 2002) and H. P. Fielder (Ed) Lexikon der Hilfsstoffe für Pharmazie, Kosmetik and angrenzende Gebiete (Edition Cantor Aulendorf, 1989)).

In some embodiments, each of the one or more pharmaceutically acceptable excipients can be independently selected from thickeners, viscosity enhancing agents, bulking agents, mucoadhesive agents, penetration enhanceers, buffers, preservatives, diluents, binders, lubricants, glidants, disintegrants, fillers, solubilizing agents, pH modifying agents, preservatives, stabilizing agents, anti-oxidants, wetting or emulsifying agents, suspending agents, pigments, colorants, isotonic agents, chelating agents, emulsifiers, and diagnostic agents.

In certain embodiments, each of the one or more pharmaceutically acceptable excipients can be independently selected from thickeners, viscosity enhancing agents, mucoadhesive agents, buffers, preservatives, diluents, binders, lubricants, glidants, disintegrants, and fillers.

In certain embodiments, each of the one or more pharmaceutically acceptable excipients can be independently selected from thickeners, viscosity enhancing agents, bulking agents, mucoadhesive agents, buffers, preservatives, and fillers.

In certain embodiments, each of the one or more pharmaceutically acceptable excipients can be independently selected from diluents, binders, lubricants, glidants, and disintegrants.

Examples of thickeners, viscosity enhancing agents, and mucoadhesive agents include without limitation: gums, e.g. xanthan gum, guar gum, locust bean gum, tragacanth gums, karaya gum, ghatti gum, cholla gum, psyllium seed gum and gum arabic; poly(carboxylic acid-containing) based polymers, such as poly (acrylic, maleic, itaconic, citraconic, hydroxyethyl methacrylic or methacrylic) acid which have strong hydrogen-bonding groups, or derivatives thereof such as salts and esters; cellulose derivatives, such as methyl cellulose, ethyl cellulose, methylethyl cellulose, hydroxymethyl cellulose, hydroxyethyl cellulose, hydroxypropyl cellulose, hydroxyethyl ethyl cellulose, carboxymethyl cellulose, hydroxypropylmethyl cellulose or cellulose esters or ethers or derivatives or salts thereof; clays such as manomorillonite clays, e.g. Veegun, attapulgite clay; polysaccharides such as dextran, pectin, amylopectin, agar, mannan or polygalactonic acid or starches such as hydroxypropyl starch or carboxymethyl starch; polypeptides such as casein, gluten, gelatin, fibrin glue; chitosan, e.g. lactate or glutamate or carboxymethyl chitin; glycosaminoglycans such as hyaluronic acid; metals or water soluble salts of alginic acid such as sodium alginate or magnesium alginate; schleroglucan; adhesives containing bismuth oxide or aluminium oxide; atherocollagen; polyvinyl polymers such as carboxyvinyl polymers; polyvinylpyrrolidone (povidone); polyvinyl alcohol; polyvinyl acetates, polyvinylmethyl ethers, polyvinyl chlorides, polyvinylidenes, and/or the like; polycarboxylated vinyl polymers such as polyacrylic acid as mentioned above; polysiloxanes; polyethers; polyethylene oxides and glycols; polyalkoxys and polyacrylamides and derivatives and salts thereof. Preferred examples can include cellulose derivatives, such as methyl cellulose, ethyl cellulose, methylethyl cellulose, hydroxymethyl cellulose, hydroxyethyl cellulose, hydroxypropyl cellulose, hydroxyethyl ethyl cellulose, carboxymethyl cellulose, hydroxypropylmethyl cellulose or cellulose esters or ethers or derivatives or salts thereof (e.g., methyl cellulose); and polyvinyl polymers such as polyvinylpyrrolidone (povidone).

Examples of preservatives include without limitation: benzalkonium chloride, benzoxonium chloride, benzethonium chloride, cetrimide, sepazonium chloride, cetylpyridinium chloride, domiphen bromide (Bradosol®), thiomersal, phenylmercuric nitrate, phenylmercuric acetate, phenylmercuric borate, methylparaben, propylparaben, chlorobutanol, benzyl alcohol, phenyl ethyl alcohol, chlorohexidine, polyhexamethylene biguanide, sodium perborate, imidazolidinyl urea, sorbic acid, Purite®), Polyquart®), and sodium perborate tetrahydrate and the like.

In certain embodiments, the preservative is a paraben, or a pharmaceutically acceptable salt thereof. In some embodiments, the paraben is an alkyl substituted 4-hydroxybenzoate, or a pharmaceutically acceptable salt or ester thereof. In certain embodiments, the alkyl is a C₁-C₄ alkyl. In certain embodiments, the preservative is methyl 4-hydroxybenzoate (methylparaben), or a pharmaceutically acceptable salt or ester thereof, propyl 4-hydroxybenzoate (propylparaben), or a pharmaceutically acceptable salt or ester thereof, or a combination thereof.

Examples of buffers include without limitation: phosphate buffer system (sodium dihydrogen phospahate dehydrate, disodium phosphate dodecahydrate, bibasic sodium phosphate, anhydrous monobasic sodium phosphate), bicarbonate buffer system, and bisulfate buffer system.

Examples of disintegrants include, without limitation: carmellose calcium, low substituted hydroxypropyl cellulose (L-HPC), carmellose, croscarmellose sodium, partially pregelatinized starch, dry starch, carboxymethyl starch sodium, crospovidone, poly sorbate 80 (polyoxyethylenesorbitan oleate), starch, sodium starch glycolate, hydroxypropyl cellulose pregelatinized starch, clays, cellulose, alginine, gums or cross linked polymers, such as cross-linked PVP (Polyplasdone XL from GAF Chemical Corp). In certain embodiments, the disintegrant is crospovidone.

Examples of glidants and lubricants (aggregation inhibitors) include without limitation: talc, magnesium stearate, calcium stearate, colloidal silica, stearic acid, aqueous silicon dioxide, synthetic magnesium silicate, fine granulated silicon oxide, starch, sodium laurylsulfate, boric acid, magnesium oxide, waxes, hydrogenated oil, polyethylene glycol, sodium benzoate, stearic acid glycerol behenate, polyethylene glycol, and mineral oil. In certain embodiments, the glidant/lubricant is magnesium stearate, talc, and/or colloidal silica; e.g., magnesium stearate and/or talc.

Examples of diluents, also referred to as “fillers” or “bulking agents” include without limitation: dicalcium phosphate dihydrate, calcium sulfate, lactose (e.g., lactose monohydrate), sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate and powdered sugar. In certain embodiments, the diluent is lactose (e.g., lactose monohydrate).

Examples of binders include without limitation: starch, pregelatinized starch, gelatin, sugars (including sucrose, glucose, dxtrose, lactose and sorbitol), polyethylene glycol, waxes, natural and synthetic gums such as acacia tragacanth, sodium alginate cellulose, including hydroxypropylmethylcellulose, hydroxypropylcellulose, ethylcellulose, and veegum, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid and polyvinylpyrrolidone (povidone). In certain embodiments, the binder is polyvinylpyrrolidone (povidone).

In some embodiments, enema formulations containing the chemical entities described herein include water and one or more (e.g., all) of the following excipients:
One or more (e.g., one, two, or three) thickeners, viscosity enhancing agents, binders, and/or mucoadhesive agents (e.g., cellulose or cellulose esters or ethers or derivatives or salts thereof (e.g., methyl cellulose); and polyvinyl polymers such as polyvinylpyrrolidone (povidone);
One or more (e.g., one or two; e.g., two) preservatives, such as a paraben, e.g., methyl 4-hydroxybenzoate (methylparaben), or a pharmaceutically acceptable salt or ester thereof, propyl 4-hydroxybenzoate (propylparaben), or a pharmaceutically acceptable salt or ester thereof, or a combination thereof;
One or more (e.g., one or two; e.g., two) buffers, such as phosphate buffer system (e.g., sodium dihydrogen phospahate dehydrate, disodium phosphate dodecahydrate);
One or more (e.g., one or two, e.g., two) glidants and/or lubricants, such as magnesium stearate and/or talc;
One or more (e.g., one or two; e.g., one) disintegrants, such as crospovidone; and
One or more (e.g., one or two; e.g., one) diluents, such as lactose (e.g., lactose monohydrate).

In certain of these embodiments, the chemical entity is a compound of Formula AA, or a pharmaceutically acceptable salt and/or hydrate and/or cocrystal thereof.

In certain embodiments, enema formulations containing the chemical entities described herein include water, methyl cellulose, povidone, methylparaben, propylparaben, sodium dihydrogen phospahate dehydrate, disodium phosphate dodecahydrate, crospovidone, lactose monohydrate, magnesium stearate, and talc. In certain of these embodiments, the chemical entity is a compound of Formula AA, or a pharmaceutically acceptable salt and/or hydrate and/or cocrystal thereof.

In certain embodiments, enema formulations containing the chemical entities described herein are provided in one or more kits or packs. In certain embodiments, the kit or pack includes two separately contained/packaged components, which when mixed together, provide the desired formulation (e.g., as a suspension). In certain of these embodiments, the two component system includes a first component and a second component, in which: (i) the first component (e.g., contained in a sachet) includes the chemical entity (as described anywhere herein) and one or more pharmaceutically acceptable excipients (e.g., together formulated as a solid preparation, e.g., together formulated as a wet granulated solid preparation); and (ii) the second component (e.g., contained in a vial or bottle) includes one or more liquids and one or more one or more other pharmaceutically acceptable excipients together forming a liquid carrier. In other embodiments, each of component (i) and (ii) is provided in its own separate kit or pack.

In certain of these embodiments, component (i) includes the chemical entitiy (e.g., a compound of Formula AA, or a pharmaceutically acceptable salt and/or hydrate and/or cocrystal thereof; e.g., a compound of Formula AA) and one or more (e.g., all) of the following excipients:
(a) One or more (e.g., one) binders (e.g., a polyvinyl polymer, such as polyvinylpyrrolidone (povidone);
(b) One or more (e.g., one or two, e.g., two) glidants and/or lubricants, such as magnesium stearate and/or talc;
(c) One or more (e.g., one or two; e.g., one) disintegrants, such as crospovidone; and
(d) One or more (e.g., one or two; e.g., one) diluents, such as lactose (e.g., lactose monohydrate).

In certain embodiments, component (i) includes from about 40 weight percent to about 80 weight percent (e.g., from about 50 weight percent to about 70 weight percent, from about 55 weight percent to about 70 weight percent; from about 60 weight percent to about 65 weight percent; e.g., about 62.1 weight percent) of the chemical entity (e.g., a compound of Formula AA, or a pharmaceutically acceptable salt and/or hydrate and/or cocrystal thereof).

In certain embodiments, component (i) includes from about 0.5 weight percent to about 5 weight percent (e.g., from about 1.5 weight percent to about 4.5 weight percent, from about 2 weight percent to about 3.5 weight percent; e.g., about 2.76 weight percent) of the binder (e.g., povidone).

In certain embodiments, component (i) includes from about 0.5 weight percent to about 5 weight percent (e.g., from about 0.5 weight percent to about 3 weight percent, from about 1 weight percent to about 3 weight percent; about 2 weight percent e.g., about 1.9 weight percent) of the disintegrant (e.g., crospovidone).

In certain embodiments, component (i) includes from about 10 weight percent to about 50 weight percent (e.g., from about 20 weight percent to about 40 weight percent, from about 25 weight percent to about 35 weight percent; e.g., about 31.03 weight percent) of the diluent (e.g., lactose, e.g., lactose monohydrate).

In certain embodiments, component (i) includes from about 0.05 weight percent to about 5 weight percent (e.g., from about 0.05 weight percent to about 3 weight percent) of the glidants and/or lubricants.

In certain embodiments (e.g., when component (i) includes one or more lubricants, such as magnesium stearate), component (i) includes from about 0.05 weight percent to about 1 weight percent (e.g., from about 0.05 weight percent to about 1 weight percent; from about 0.1 weight percent to about 1 weight percent; from about 0.1 weight percent to about 0.5 weight percent; e.g., about 0.27 weight percent) of the lubricant (e.g., magnesium stearate).

In certain embodiments (when component (i) includes one or more lubricants, such as talc), component (i) includesfrom about 0.5 weight percent to about 5 weight percent (e.g., from about 0.5 weight percent to about 3 weight percent, from about 1 weight percent to about 3 weight percent; from about 1.5 weight percent to about 2.5 weight percent; from about 1.8 weight percent to about 2.2 weight percent; about 1.93 weight percent) of the lubricant (e.g., talc).

In certain of these embodiments, each of (a), (b), (c), and (d) above is present.

In certain embodiments, component (i) includes the ingredients and amounts as shown in Table A.

TABLE A

| Ingredient | Weight Percent |
|---|---|
| A compound of Formula | 40 weight percent to about 80 weight |
| AA | percent (e.g., from about 50 weight percent |
| | to about 70 weight percent, from about 55 |
| | weight percent to about 70 weight percent; |
| | from about 60 weight percent to about 65 |
| | weight percent; e.g., about 62.1 weight |
| | percent) |
| Crospovidone (Kollidon | 0.5 weight percent to about 5 weight |
| CL) | percent (e.g., from about 0.5 weight |
| | percent to about 3 weight percent, from |
| | about 1 weight percent to about 3 weight |
| | percent; about 1.93 weight percent |
| lactose monohydrate | about 10 weight percent to about 50 weight |
| (Pharmatose 200M) | percent (e.g., from about 20 weight percent |
| | to about 40 weight percent, from about 25 |
| | weight percent to about 35 weight percent; |
| | e.g., about 31.03 weight percent |
| Povidone (Kollidon K30) | about 0.5 weight percent to about 5 weight |
| | percent (e.g., from about 1.5 weight |
| | percent to about 4.5 weight percent, from |
| | about 2 weight percent to about 3.5 weight |
| | percent; e.g., about 2.76 weight percent |
| talc | 0.5 weight percent to about 5 weight |
| | percent (e.g., from about 0.5 weight |
| | percent to about 3 weight percent, from |
| | about 1 weight percent to about 3 weight |
| | percent; from about 1.5 weight percent to |
| | about 2.5 weight percent; from about 1.8 |
| | weight percent to about 2.2 weight |
| | percent; e.g., about 1.93 weight percent |
| Magnesium stearate | about 0.05 weight percent to about 1 |
| | weight percent (e.g., from about 0.05 |
| | weight percent to about 1 weight percent; |
| | from about 0.1 weight percent to about 1 |
| | weight percent; from about 0.1 weight |
| | percent to about 0.5 weight percent; e.g., |
| | about 0.27 weight percent |

In certain embodiments, component (1) includes the ingredients and amounts as shown in Table B.

TABLE B

| Ingredient | Weight Percent |
|---|---|
| A compound of Formula AA | About 62.1 weight percent) |
| Crospovidone (Kollidon CL) | About 1.93 weight percent |
| lactose monohydrate (Pharmatose 200M) | About 31.03 weight percent |
| Povidone (Kollidon K30) | About 2.76 weight percent |
| talc | About 1.93 weight percent |
| Magnesium stearate | About 0.27 weight percent |

In certain embodiments, component (i) is formulated as a wet granulated solid preparation. In certain of these embodiments an internal phase of ingredients (the chemical entity, disintegrant, and diluent) are combined and mixed in a high-shear granulator. A binder (e.g., povidone) is dissolved in water to form a granulating solution. This solution is added to the Inner Phase mixture resulting in the development of granules. While not wishing to be bound by theory, granule development is believed to be facilitated by the interaction of the polymeric binder with the materials of the internal phase. Once the granulation is formed and dried, an external phase (e.g., one or more lubricants—not an intrinsic component of the dried granulation), is added to the dry granulation. It is believed that lubrication of the granulation is important to the flowability of the granulation, in particular for packaging.

In certain of the foregoing embodiments, component (ii) includes water and one or more (e.g., all) of the following excipients:
(a′) One or more (e.g., one, two; e.g., two) thickeners, viscosity enhancing agents, binders, and/or mucoadhesive agents (e.g., cellulose or cellulose esters or ethers or derivatives or salts thereof (e.g., methyl cellulose); and polyvinyl polymers such as polyvinylpyrrolidone (povidone);
(b′) One or more (e.g., one or two; e.g., two) preservatives, such as a paraben, e.g., methyl 4-hydroxybenzoate (methylparaben), or a pharmaceutically acceptable salt or ester thereof, propyl 4-hydroxybenzoate (propylparaben), or a pharmaceutically acceptable salt or ester thereof, or a combination thereof; and
(c′) One or more (e.g., one or two; e.g., two) buffers, such as phosphate buffer system (e.g., sodium dihydrogen phospahate dihydrate, disodium phosphate dodecahydrate);

In certain of the foregoing embodiments, component (ii) includes water and one or more (e.g., all) of the following excipients:
(a″) a first thickener, viscosity enhancing agent, binder, and/or mucoadhesive agent (e.g., a cellulose or cellulose ester or ether or derivative or salt thereof (e.g., methyl cellulose));
(a″′) a second thickener, viscosity enhancing agent, binder, and/or mucoadhesive agent (e.g., a polyvinyl polymer, such as polyvinylpyrrolidone (povidone));
(b″) a first preservative, such as a paraben, e.g., propyl 4-hydroxybenzoate (propylparaben), or a pharmaceutically acceptable salt or ester thereof;
(b″) a second preservative, such as a paraben, e.g., methyl 4-hydroxybenzoate (methylparaben), or a pharmaceutically acceptable salt or ester thereof,
(c″) a first buffer, such as phosphate buffer system (e.g., disodium phosphate dodecahydrate);
(c′″) a second buffer, such as phosphate buffer system (e.g., sodium dihydrogen phospahate dehydrate),

In certain embodiments, component (ii) includes from about 0.05 weight percent to about 5 weight percent (e.g., from about 0.05 weight percent to about 3 weight percent, from about 0.1 weight percent to about 3 weight percent; e.g., about 1.4 weight percent) of (a″).

In certain embodiments, component (ii) includes from about 0.05 weight percent to about 5 weight percent (e.g., from about 0.05 weight percent to about 3 weight percent, from about 0.1 weight percent to about 2 weight percent; e.g., about 1.0 weight percent) of (a″′).

In certain embodiments, component (ii) includes from about 0.005 weight percent to about 0.1 weight percent (e.g., from about 0.005 weight percent to about 0.05 weight percent; e.g., about 0.02 weight percent) of (b″).

In certain embodiments, component (ii) includes from about 0.05 weight percent to about 1 weight percent (e.g., from about 0.05 weight percent to about 0.5 weight percent; e.g., about 0.20 weight percent) of (b″′).

In certain embodiments, component (ii) includes from about 0.05 weight percent to about 1 weight percent (e.g., from about 0.05 weight percent to about 0.5 weight percent; e.g., about 0.15 weight percent) of (c″).

In certain embodiments, component (ii) includes from about 0.005 weight percent to about 0.5 weight percent (e.g., from about 0.005 weight percent to about 0.3 weight percent; e.g., about 0.15 weight percent) of (c′″).

In certain of these embodiments, each of (a″)-(c′) is present.

In certain embodiments, component (ii) includes water (up to 100%) and the ingredients and amounts as shown in Table C.

TABLE C

| Ingredient | Weight Percent |
|---|---|
| methyl cellulose | 0.05 weight percent to about 5 weight |
| (Methocel A15C premium) | percent (e.g., from about 0.05 weight |
| | percent to about 3 weight percent, from |
| | about 0.1 weight percent to about 3 weight |
| | percent; e.g., about 1.4 weight percent |
| Povidone (Kollidon K30) | 0.05 weight percent to about 5 weight |
| | percent (e.g., from about 0.05 weight |
| | percent to about 3 weight percent, from |
| | about 0.1 weight percent to about 2 weight |
| | percent; e.g., about 1.0 weight percent |
| propyl 4-hydroxybenzoate | about 0.005 weight percent to about 0.1 |
| | weight percent (e.g., from about 0.005 |
| | weight percent to about 0.05 weight |
| | percent; e.g., about 0.02 weight percent) |
| methyl 4-hydroxybenzoate | about 0.05 weight percent to about 1 |
| | weight percent (e.g., from about 0.05 |
| | weight percent to about 0.5 weight percent; |
| | e.g., about 0.20 weight percent) |
| disodium phosphate | about 0.05 weight percent to about 1 |
| dodecahydrate | weight percent (e.g., from about 0.05 |
| | weight percent to about 0.5 weight percent; |
| | e.g., about 0.15 weight percent) |
| sodium dihydrogen | about 0.005 weight percent to about 0.5 |
| phospahate dihydrate | weight percent (e.g., from about 0.005 |
| | weight percent to about 0.3 weight percent; |
| | e.g., about 0.15 weight percent) |

In certain embodiments, component (ii) includes water (up to 100%) and the ingredients and amounts as shown in Table D.

TABLE D

| Ingredient | Weight Percent |
|---|---|
| methyl cellulose (Methocel A15C premium) | about 1.4 weight percent |
| Povidone (Kollidon K30) | about 1.0 weight percent |
| propyl 4-hydroxybenzoate | about 0.02 weight percent |
| methyl 4-hydroxybenzoate | about 0.20 weight percent |
| disodium phosphate dodecahydrate | about 0.15 weight percent |
| sodium dihydrogen phospahate dihydrate | about 0.15 weight percent |

Ready-to-use” enemas are generally be provided in a “single-use” sealed disposable container of plastic or glass. Those formed of a polymeric material preferably have sufficient flexibility for ease of use by an unassisted patient. Typical plastic containers can be made of polyethylene. These containers may comprise a tip for direct introduction into the rectum. Such containers may also comprise a tube between the container and the tip. The tip is preferably provided with a protective shield which is removed before use. Optionally the tip has a lubricant to improve patient compliance.

In some embodiments, the enema formulation (e.g., suspension) is poured into a bottle for delivery after it has been prepared in a separate container. In certain embodiments, the bottle is a plastic bottle (e.g., flexible to allow for delivery by squeezing the bottle), which can be a polyethylene bottle (e.g., white in color). In some embodiments, the bottle is a single chamber bottle, which contains the suspension or solution. In other embodiments, the bottle is a multichamber bottle, where each chamber contains a separate mixture or solution. In still other embodiments, the bottle can further include a tip or rectal cannula for direct introduction into the rectum.

Dosages

The dosages may be varied depending on the requirement of the patient, the severity of the condition being treating and the particular compound being employed. Determination of the proper dosage for a particular situation can be determined by one skilled in the medical arts. The total daily dosage may be divided and administered in portions throughout the day or by means providing continuous delivery.

In some embodiments, the compounds described herein are administered at a dosage of from about 0.001 mg/Kg to about 500 mg/Kg (e.g., from about 0.001 mg/Kg to about 200 mg/Kg; from about 0.01 mg/Kg to about 200 mg/Kg; from about 0.01 mg/Kg to about 150 mg/Kg; from about 0.01 mg/Kg to about 100 mg/Kg; from about 0.01 mg/Kg to about 50 mg/Kg; from about 0.01 mg/Kg to about 10 mg/Kg; from about 0.01 mg/Kg to about 5 mg/Kg; from about 0.01 mg/Kg to about 1 mg/Kg; from about 0.01 mg/Kg to about 0.5 mg/Kg; from about 0.01 mg/Kg to about 0.1 mg/Kg; from about 0.1 mg/Kg to about 200 mg/Kg; from about 0.1 mg/Kg to about 150 mg/Kg; from about 0.1 mg/Kg to about 100 mg/Kg; from about 0.1 mg/Kg to about 50 mg/Kg; from about 0.1 mg/Kg to about 10 mg/Kg; from about 0.1 mg/Kg to about 5 mg/Kg; from about 0.1 mg/Kg to about 1 mg/Kg; from about 0.1 mg/Kg to about 0.5 mg/Kg).

In some embodiments, enema formulations include from about 0.5 mg to about 2500 mg (e.g., from about 0.5 mg to about 2000 mg, from about 0.5 mg to about 1000 mg, from about 0.5 mg to about 750 mg, from about 0.5 mg to about 600 mg, from about 0.5 mg to about 500 mg, from about 0.5 mg to about 400 mg, from about 0.5 mg to about 300 mg, from about 0.5 mg to about 200 mg; e.g., from about 5 mg to about 2500 mg, from about 5 mg to about 2000 mg, from about 5 mg to about 1000 mg; from about 5 mg to about 750 mg; from about 5 mg to about 600 mg; from about 5 mg to about 500 mg; from about 5 mg to about 400 mg; from about 5 mg to about 300 mg; from about 5 mg to about 200 mg; e.g., from about 50 mg to about 2000 mg, from about 50 mg to about 1000 mg, from about 50 mg to about 750 mg, from about 50 mg to about 600 mg, from about 50 mg to about 500 mg, from about 50 mg to about 400 mg, from about 50 mg to about 300 mg, from about 50 mg to about 200 mg; e.g., from about 100 mg to about 2500 mg, from about 100 mg to about 2000 mg, from about 100 mg to about 1000 mg, from about 100 mg to about 750 mg, from about 100 mg to about 700 mg, from about 100 mg to about 600 mg, from about 100 mg to about 500 mg, from about 100 mg to about 400 mg, from about 100 mg to about 300 mg, from about 100 mg to about 200 mg; e.g., from about 150 mg to about 2500 mg, from about 150 mg to about 2000 mg, from about 150 mg to about 1000 mg, from about 150 mg to about 750 mg, from about 150 mg to about 700 mg, from about 150 mg to about 600 mg, from about 150 mg to about 500 mg, from about 150 mg to about 400 mg, from about 150 mg to about 300 mg, from about 150 mg to about 200 mg; e.g., from about 150 mg to about 500 mg; e.g., from about 300 mg to about 2500 mg, from about 300 mg to about 2000 mg, from about 300 mg to about 1000 mg, from about 300 mg to about 750 mg, from about 300 mg to about 700 mg, from about 300 mg to about 600 mg; e.g., from about 400 mg to about 2500 mg, from about 400 mg to about 2000 mg, from about 400 mg to about 1000 mg, from about 400 mg to about 750 mg, from about 400 mg to about 700 mg, from about 400 mg to about 600 from about 400 mg to about 500 mg; e.g., 150 mg or 450 mg) of the chemical entity in from about 1 mL to about 3000 mL (e.g., from about 1 mL to about 2000 mL, from about 1 mL to about 1000 mL, from about 1 mL to about 500 mL, from about 1 mL to about 250 mL, from about 1 mL to about 100 mL, from about 10 mL to about 1000 mL, from about 10 mL to about 500 mL, from about 10 mL to about 250 mL, from about 10 mL to about 100 mL, from about 30 mL to about 90 mL, from about 40 mL to about 80 mL; from about 50 mL to about 70 mL; e.g., about 1 mL, about 5 mL, about 10 mL, about 15 mL, about 20 mL, about 25 mL, about 30 mL, about 35 mL, about 40 mL, about 45 mL, about 50 mL, about 55 mL, about 60 mL, about 65 mL, about 70 mL, about 75 mL, about 100 mL, about 250 mL, or about 500 mL, or about 1000 mL, or about 2000 mL, or about 3000 mL; e.g., 60 mL) of liquid carrier.

In certain embodiments, enema formulations include from about 50 mg to about 250 mg (e.g., from about 100 mg to about 200; e.g., about 150 mg) of the chemical entity in from about 10 mL to about 100 mL (e.g., from about 20 mL to about 100 mL, from about 30 mL to about 90 mL, from about 40 mL to about 80 mL; from about 50 mL to about 70 mL) of liquid carrier. In certain embodiments, enema formulations include about 150 mg of the chemical entity in about 60 mL of the liquid carrier. In certain of these embodiments, the chemical entity is a compound of Formula AA, or a pharmaceutically acceptable salt and/or hydrate and/or cocrystal thereof. For example, enema formulations can include about 150 mg of a compound of Formula AA in about 60 mL of the liquid carrier.

In certain embodiments, enema formulations include from about 350 mg to about 550 mg (e.g., from about 400 mg to about 500; e.g., about 450 mg) of the chemical entity in from about 10 mL to about 100 mL (e.g., from about 20 mL to about 100 mL, from about 30 mL to about 90 mL, from about 40 mL to about 80 mL; from about 50 mL to about 70 mL) of liquid carrier. In certain embodiments, enema formulations include about 450 mg of the chemical entity in about 60 mL of the liquid carrier. In certain of these embodiments, the chemical entity is a compound of Formula

AA, or a pharmaceutically acceptable salt and/or hydrate and/or cocrystal thereof. For example, enema formulations can include about 450 mg of a compound of Formula AA in about 60 mL of the liquid carrier.

In some embodiments, enema formulations include from about from about 0.01 mg/mL to about 50 mg/mL (e.g., from about 0.01 mg/mL to about 25 mg/mL; from about 0.01 mg/mL to about 10 mg/mL; from about 0.01 mg/mL to about 5 mg/mL; from about 0.1 mg/mL to about 50 mg/mL; from about 0.01 mg/mL to about 25 mg/mL; from about 0.1 mg/mL to about 10 mg/mL; from about 0.1 mg/mL to about 5 mg/mL; from about 1 mg/mL to about 10 mg/mL; from about 1 mg/mL to about 5 mg/mL; from about 5 mg/mL to about 10 mg/mL; e.g., about 2.5 mg/mL or about 7.5 mg/mL) of the chemical entity in liquid carrier. In certain of these embodiments, the chemical entity is a compound of Formula AA, or a pharmaceutically acceptable salt and/or hydrate and/or cocrystal thereof. For example, enema formulations can include about 2.5 mg/mL or about 7.5 mg/mL of a compound of Formula AA in liquid carrier.

Regimens

The foregoing dosages can be administered on a daily basis (e.g., as a single dose or as two or more divided doses) or non-daily basis (e.g., every other day, every two days, every three days, once weekly, twice weeks, once every two weeks, once a month).

In some embodiments, the period of administration of a compound described herein is for 1 day, 2 days, 3 days, 4 days, 5 days, 6 days, 7 days, 8 days, 9 days, 10 days, 1 1 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In a further embodiment, a period of during which administration is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In an embodiment, a therapeutic compound is administered to an individual for a period of time followed by a separate period of time. In another embodiment, a therapeutic compound is administered for a first period and a second period following the first period, with administration stopped during the second period, followed by a third period where administration of the therapeutic compound is started and then a fourth period following the third period where administration is stopped. In an aspect of this embodiment, the period of administration of a therapeutic compound followed by a period where administration is stopped is repeated for a determined or undetermined period of time. In a further embodiment, a period of administration is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In a further embodiment, a period of during which administration is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more.

Methods of Treatment

In some embodiments, methods for treating a subject having condition, disease or disorder in which a decrease or increase in NLRP3 activity (e.g., an increase, e.g., NLRP3 signaling) contributes to the pathology and/or symptoms and/or progression of the condition, disease or disorder are provided, comprising administering to a subject an effective amount of a chemical entity described herein (e.g., a compound described generically or specifically herein or a pharmaceutically acceptable salt thereof or compositions containing the same).

Indications

In some embodiments, the condition, disease or disorder is selected from: inappropriate host responses to infectious diseases where active infection exists at any body site, such as septic shock, disseminated intravascular coagulation, and/or adult respiratory distress syndrome; acute or chronic inflammation due to antigen, antibody and/or complement deposition; inflammatory conditions including arthritis, cholangitis, colitis, encephalitis, endocarditis, glomerulonephritis, hepatitis, myocarditis, pancreatitis, pericarditis, reperfusion injury and vasculitis, immune-based diseases such as acute and delayed hypersensitivity, graft rejection, and graft-versus-host disease; auto-immune diseases including Type 1 diabetes mellitus and multiple sclerosis. For example, the condition, disease or disorder may be an inflammatory disorder such as rheumatoid arthritis, osteoarthritis, septic shock, COPD and periodontal disease.

In some embodiments, the condition, disease or disorder is an autoimmune diseases. Non-limiting examples include rheumatoid arthritis, systemic lupus erythematosus, multiple sclerosis, inflammatory bowel diseases (IBDs) comprising Crohn disease (CD) and ulcerative colitis (UC), which are chronic inflammatory conditions with polygenic susceptibility. In certain embodiments, the condition is an inflammatory bowel disease. In certain embodiments, the condition is Crohn's disease, autoimmune colitis, iatrogenic autoimmune colitis, ulcerative colitis, colitis induced by one or more chemotherapeutic agents, colitis induced by treatment with adoptive cell therapy, colitis associated by one or more alloimmune diseases (such as graft-vs-host disease, e.g., acute graft vs. host disease and chronic graft vs. host disease), radiation enteritis, collagenous colitis, lymphocytic colitis, microscopic colitis, and radiation enteritis. In certain of these embodiments, the condition is alloimmune disease (such as graft-vs-host disease, e.g., acute graft vs. host disease and chronic graft vs. host disease), celiac disease, irritable bowel syndrome, rheumatoid arthritis, lupus, scleroderma, psoriasis, cutaneous T-cell lymphoma, uveitis, and mucositis (e.g., oral mucositis, esophageal mucositis or intestinal mucositis).

In some embodiments, the condition, disease or disorder is selected from major adverse cardiovascular events such as carbiovascular death, non-fatal myocardial infarction and non-fatal stroke in patients with a prior hear attack and inflammatory atherosclerosis (see for example, NCT01327846).

In some embodiments, the condition, disease or disorder is selected from metabolic disorders such as type 2 diabetes, atherosclerosis, obesity and gout, as well as diseases of the central nervous system, such as Alzheimer's disease and multiple sclerosis and Amyotrophic Lateral Sclerosis and Parkinson disease, lung disease, such as asthma and COPD and pulmonary idiopathic fibrosis, liver disease, such as NASH syndrome, viral hepatitis and cirrhosis, pancreatic disease, such as acute and chronic pancreatitis, kidney disease, such as acute and chronic kidney injury, intestinal disease such as Crohn's disease and Ulcerative Colitis, skin disease such as psoriasis, musculoskeletal disease such as scleroderma, vessel disorders, such as giant cell arteritis, disorders of the bones, such as Osteoarthritis , osteoporosis and osteopetrosis disorders eye disease, such as glaucoma and macular degeneration, diseased caused by viral infection such as HIV and AIDS, autoimmune disease such as Rheumatoid Arthritis, Systemic Lupus Erythematosus, Autoimmune Thyroiditis, Addison's disease, pernicious anemia, cancer and aging.

In some embodiments, the condition, disease or disorder is a cardiovascular indication. In some embodiments, the condition, disease or disorder is myocardial infraction. In some embodiments, the condition, disease or disorder is stroke.

In some embodiments, the condition, disease or disorder is obesity.

In some embodiments, the condition, disease or disorder is Type 2 Diabetes.

In some embodiments, the condition, disease or disorder is NASH.

In some embodiments, the condition, disease or disorder is Alzheimer's disease.

In some embodiments, the condition, disease or disorder is gout.

In some embodiments, the condition, disease or disorder is SLE.

In some embodiments, the condition, disease or disorder is rheumatoid arthritis.

In some embodiments, the condition, disease or disorder is IBD.

In some embodiments, the condition, disease or disorder is multiple sclerosis.

In some embodiments, the condition, disease or disorder is COPD.

In some embodiments, the condition, disease or disorder is asthma.

In some embodiments, the condition, disease or disorder is scleroderma.

In some embodiments, the condition, disease or disorder is pulmonary fibrosis.

In some embodiments, the condition, disease or disorder is age related macular degeneration (AMD).

In some embodiments, the condition, disease or disorder is cystic fibrosis.

In some embodiments, the condition, disease or disorder is Muckle Wells syndrome.

In some embodiments, the condition, disease or disorder is familial cold autoinflammatory syndrome (FCAS).

In some embodiments, the condition, disease or disorder is chronic neurologic cutaneous and articular syndrome.

In some embodiments, the condition, disease or disorder is selected from: myelodysplastic syndromes (MDS); non-small cell lung cancer, such as non-small cell lung cancer in patients carrying mutation or overexpression of NLRP3; acute lymphoblastic leukemia (ALL), such as ALL in patients resistant to glucocorticoids treatment; Langerhan's cell histiocytosis (LCH); multiple myeloma; promyelocytic leukemia; acute myeloid leukemia (AML) chronic myeloid leukemia (CML); gastric cancer; and lung cancer metastasis.

In some embodiments, the condition, disease or disorder is selected from: myelodysplastic syndromes (MDS); non-small cell lung cancer, such as non-small cell lung cancer in patients carrying mutation or overexpression of NLRP3; acute lymphoblastic leukemia (ALL), such as ALL in patients resistant to glucocorticoids treatment; Langerhan's cell histiocytosis (LCH); multiple myeloma; promyelocytic leukemia; gastric cancer; and lung cancer metastasis.

In some embodiments, the indication is MDS.

In some embodiments, the indication is non-small lung cancer in patients carrying mutation or overexpression of NLRP3.

In some embodiments, the indication is ALL in patients resistant to glucocorticoids treatment.

In some embodiments, the indication is LCH.

In some embodiments, the indication is multiple myeloma.

In some embodiments, the indication is promyelocytic leukemia.

In some embodiments, the indication is gastric cancer.

In some embodiments, the indication is lung cancer metastasis.

Combination Therapy

This disclosure contemplates both monotherapy regimens as well as combination therapy regimens.

In some embodiments, the methods described herein can further include administering one or more additional therapies (e.g., one or more additional therapeutic agents and/or one or more therapeutic regimens) in combination with administration of the compounds described herein.

In certain embodiments, the second therapeutic agent or regimen is administered to the subject prior to contacting with or administering the chemical entity (e.g., about one hour prior, or about 6 hours prior, or about 12 hours prior, or about 24 hours prior, or about 48 hours prior, or about 1 week prior, or about 1 month prior).

In other embodiments, the second therapeutic agent or regimen is administered to the subject at about the same time as contacting with or administering the chemical entity. By way of example, the second therapeutic agent or regimen and the chemical entity are provided to the subject simultaneously in the same dosage form. As another example, the second therapeutic agent or regimen and the chemical entity are provided to the subject concurrently in separate dosage forms.

In still other embodiments, the second therapeutic agent or regimen is administered to the subject after contacting with or administering the chemical entity (e.g., about one hour after, or about 6 hours after, or about 12 hours after, or about 24 hours after, or about 48 hours after, or about 1 week after, or about 1 month after).

Patient Selection

In some embodiments, the methods described herein further include the step of identifying a subject (e.g., a patient) in need of treatment for an indication related to NLRP3 activity, such as an indication related to NLRP3 polymorphism.

In some embodiments, the methods described herein further include the step of identifying a subject (e.g., a patient) in need of treatment for an indication related to NLRP3 activity, such as an indication related to NLRP3 where polymorphism is a gain of function

In some embodiments, the methods described herein further include the step of identifying a subject (e.g., a patient) in need of treatment for an indication related to NLRP3 activity, such as an indication related to NLRP3 polymorphism found in CAPS syndromes.

In some embodiments, the methods described herein further include the step of identifying a subject (e.g., a patient) in need of treatment for an indication related to NLRP3 activity, such as an indication related NLRP3 polymorphism where the polymorphism is VAR_014104 (R262W)

In some embodiments, the methods described herein further include the step of identifying a subject (e.g., a patient) in need of treatment for an indication related to NLRP3 activity, such as an indication related NLRP3 polymorphism where the polymorphism is a natural variant reported in http://www.uniprot.org/uniprot/Q96P20.

In some embodiments, the methods described herein further include the step of identifying a subject (e.g., a patient) in need of treatment for an indication related to NLRP3 activity, such as an indication related to point mutation of NLRP3 signaling.

### Anti-TNFα Agents

The term “anti-TNFα agent” refers to an agent which directly or indirectly blocks, down-regulates, impairs, inhibits, impairs, or reduces TNFα activity and/or expression. In some embodiments, an anti-TNFα agent is an antibody or an antigen-binding fragment thereof, a fusion protein, a soluble TNFα receptor (a soluble tumor necrosis factor receptor superfamily member 1A (TNFR1) or a soluble tumor necrosis factor receptor superfamily 1B (TNFR2)), an inhibitory nucleic acid, or a small molecule TNFα antagonist. In some embodiments, the inhibitory nucleic acid is a ribozyme, small hairpin RNA, a small interfering RNA, an antisense nucleic acid, or an aptamer.

Exemplary anti-TNFα agents that directly block, down-regulate, impair, inhibit, or reduce TNFα activity and/or expression can, e.g., inhibit or decrease the expression level of TNFα or a receptor of TNFα (TNFR1 or TNFR2) in a cell (e.g., a cell obtained from a subject, a mammalian cell), or inhibit or reduce binding of TNFα to its receptor (TNFR1 and/or TNFR2) and/or. Non-limiting examples of anti-TNFα agents that directly block, down-regulate, impair, inhibit, or reduce TNFα activity and/or expression include an antibody or fragment thereof, a fusion protein, a soluble TNFα receptor (e.g., a soluble TNFR1 or soluble TNFR2), inhibitory nucleic acids (e.g., any of the examples of inhibitory nucleic acids described herein), and a small molecule TNFα antagonist.

Exemplary anti-TNFα agents that can indirectly block, down-regulate, impair, inhibitreduce TNFα activity and/or expression can, e.g., inhibit or decrease the level of downstream signaling of a TNFα receptor (e.g., TNFR1 or TNFR2) in a mammalian cell (e.g., decrease the level and/or activity of one or more of the following signaling proteins: AP-1, mitogen-activated protein kinase kinase kinase 5 (ASK1), inhibitor of nuclear factor kappa B (IKK), mitogen-activated protein kinase 8 (JNK), mitogen-activated protein kinase (MAPK), MEKK 1/4, MEKK 4/7, MEKK 3/6, nuclear factor kappa B (NF-κB), mitogen-activated protein kinase kinase kinase 14 (NIK), receptor interacting serine/threonine kinase 1 (RIP), TNFRSF1A associated via death domain (TRADD), and TNF receptor associated factor 2 (TRAF2), in a cell), and/or decrease the level of TNFα-induced gene expression in a mammalian cell (e.g., decrease the transcription of genes regulated by, e.g., one or more transcription factors selected from the group of activating transcription factor 2 (ATF2), c-Jun, and NF-κB). A description of downstream signaling of a TNFα receptor is provided in Wajant et al., Cell Death Differentiation 10:45-65, 2003 (incorporated herein by reference). For example, such indirect anti-TNFα agents can be an inhibitory nucleic acid that targets (decreases the expression) a signaling component downstream of a TNFα-induced gene (e.g., any TNFα-induced gene known in the art), a TNFα receptor (e.g., any one or more of the signaling components downstream of a TNFα receptor described herein or known in the art), or a transcription factor selected from the group of NF-κB, c-Jun, and ATF2.

In other examples, such indirect anti-TNFα agents can be a small molecule inhibitor of a protein encoded by a TNFα-induced gene (e.g., any protein encoded by a TNFα-induced gene known in the art), a small molecule inhibitor of a signaling component downstream of a TNFα receptor (e.g., any of the signaling components downstream of a TNFα receptor described herein or known in the art), and a small molecule inhibitor of a transcription factor selected from the group of ATF2, c-Jun, and NF-κB.

In other embodiments, anti-TNFα agents that can indirectly block, down-regulate, impair, or reduce one or more components in a cell (e.g., a cell obtained from a subject, a mammalian cell) that are involved in the signaling pathway that results in TNFα mRNA transcription, TNFα mRNA stabilization, and TNFα mRNA translation (e.g., one or more components selected from the group of CD14, c-Jun, ERK1/2, IKK, IκB, interleukin 1 receptor associated kinase 1 (IRAK), JNK, lipopolysaccharide binding protein (LBP), MEK1/2, MEK3/6, MEK4/7, MK2, MyD88, NF-κB, NIK, PKR, p38, AKT serine/threonine kinase 1 (rac), raf kinase (raf), ras, TRAF6, TTP). For example, such indirect anti-TNFα agents can be an inhibitory nucleic acid that targets (decreases the expression) of a component in a mammalian cell that is involved in the signaling pathway that results in TNFα mRNA transcription, TNFα mRNA stabilization, and TNFα mRNA translation (e.g., a component selected from the group of CD14, c-Jun, ERK1/2, IKK, IκB, IRAK, JNK, LBP, MEK1/2, MEK3/6, MEK4/7, MK2, MyD88, NF-κB, NIK, IRAK, lipopolysaccharide binding protein (LBP), PKR, p38, rac, raf, ras, TRAF6, TTP). In other examples, an indirect anti-TNFα agents is a small molecule inhibitor of a component in a mammalian cell that is involved in the signaling pathway that results in TNFα mRNA transcription, TNFα mRNA stabilization, and TNFα mRNA translation (e.g., a component selected from the group of CD14, c-Jun, ERK1/2, IKK, IκB, IRAK, JNK, lipopolysaccharide binding protein (LBP), MEK1/2, MEK3/6, MEK4/7, MK2, MyD88, NF-κB, NIK, IRAK, lipopolysaccharide binding protein (LBP), PKR, p38, rac, raf, ras, TRAF6, TTP).

### Antibodies

In some embodiments, the anti-TNFα agent is an antibody or an antigen-binding fragment thereof (e.g., a Fab or a scFv). In some embodiments, an antibody or antigen-binding fragment of an antibody described herein can bind specifically to TNFα. In some embodiments, an antibody or antigen-binding fragment described herein binds specifically to any one of TNFα, TNFR1, or TNFR2. In some embodiments, an antibody or antigen-binding fragment of an antibody described herein can bind specifically to a TNFα receptor (TNFR1 or TNFR2).

In some embodiments, the antibody can be a humanized antibody, a chimeric antibody, a multivalent antibody, or a fragment thereof. In some embodiments, an antibody can be a scFv-Fc, a VHH domain, a VNAR domain, a (scFv)2, a minibody, or a BiTE.

In some embodiments, an antibody can be a crossmab, a diabody, a scDiabody, a scDiabody-CH3, a Diabody-CH3, a DutaMab, a DT-IgG, a diabody-Fc, a scDiabody-HAS, a charge pair antibody, a Fab-arm exchange antibody, a SEEDbody, a Triomab, a LUZ-Y, a Fcab, a kλ-body, an orthogonal Fab, a DVD-IgG, an IgG(H)-scFv, a scFv-(H)IgG, an IgG(L)-scFv, a scFv-(L)-IgG, an IgG (L,H)-Fc, an IgG(H)-V, a V(H)-IgG, an IgG(L)-V, a V(L)-IgG, an KIH IgG-scFab, a 2scFv-IgG, an IgG-2scFv, a scFv4-Ig, a Zybody, a DVI-IgG, a nanobody, a nanobody-HSA, a DVD-Ig, a dual-affinity re-targeting antibody (DART), a triomab, a kih IgG with a common LC, an ortho-Fab IgG, a 2-in-1-IgG, IgG-ScFv, scFv2-Fc, a bi-nanobody, tanden antibody, a DART-Fc, a scFv-HAS-scFv, a DAF (two-in-one or four-in-one), a DNL-Fab3, knobs-in-holes common LC, knobs-in-holes assembly, a TandAb, a Triple Body, a miniantibody, a minibody, a TriBi minibody, a scFv-CH3 KIH, a Fab-scFv, a scFv-CH-CL-scFv, a F(ab′)2-scFV2, a scFv-KIH, a Fab-scFv-Fc, a tetravalent HCAb, a scDiabody-Fc, a tandem scFv-Fc, an intrabody, a dock and lock bispecific antibody, an ImmTAC, a HSAbody, a tandem scFv, an IgG-IgG, a Cov-X-Body, and a scFv1-PEG-scFv2.

Non-limiting examples of an antigen-binding fragment of an antibody include an Fv fragment, a Fab fragment, a F(ab′)2 fragment, and a Fab′ fragment. Additional examples of an antigen-binding fragment of an antibody is an antigen-binding fragment of an antigen-binding fragment of an IgA (e.g., an antigen-binding fragment of IgAl or IgA2) (e.g., an antigen-binding fragment of a human or humanized IgA, e.g., a human or humanized IgA1 or IgA2); an antigen-binding fragment of an IgD (e.g., an antigen-binding fragment of a human or humanized IgD); an antigen-binding fragment of an IgE (e.g., an antigen-binding fragment of a human or humanized IgE); an IgG (e.g., an antigen-binding fragment of IgG1, IgG2, IgG3, or IgG4) (e.g., an antigen-binding fragment of a human or humanized IgG, e.g., human or humanized IgG1, IgG2, IgG3, or IgG4); or an antigen-binding fragment of an IgM (e.g., an antigen-binding fragment of a human or humanized IgM).

Non-limiting examples of anti-TNFα agents that are antibodies that specifically bind to TNFα are described in Ben-Horin et al., Autoimmunity Rev. 13(1):24-30, 2014; Bongartz et al., JAMA 295(19):2275-2285, 2006; Butler et al., Eur. Cytokine Network 6(4):225-230, 1994; Cohen et al., Canadian J. Gastroenterol. Hepatol. 15(6):376-384, 2001; Elliott et al., Lancet 1994; 344: 1125-1127, 1994; Feldmann et al., Ann. Rev. Immunol. 19(1):163-196, 2001; Rankin et al., Br. J. Rheumatol. 2:334-342, 1995; Knight et al., Molecular Immunol. 30(16):1443-1453, 1993; Lorenz et al., J. Immunol. 156(4):1646-1653, 1996; Hinshaw et al., Circulatory Shock 30(3):279-292, 1990; Ordas et al., Clin. Pharmacol. Therapeutics 91(4):635-646, 2012; Feldman, Nature Reviews Immunol. 2(5):364-371, 2002; Taylor et al., Nature Reviews Rheumatol. 5(10):578-582, 2009; Garces et al., Annals Rheumatic Dis. 72(12):1947-1955, 2013; Palladino et al., Nature Rev. Drug Discovery 2(9):736-746, 2003; Sandborn et al., Inflammatory Bowel Diseases 5(2):119-133, 1999; Atzeni et al., Autoimmunity Reviews 12(7):703-708, 2013; Maini et al., Immunol. Rev. 144(1):195-223, 1995; Wanner et al., Shock 11(6):391-395, 1999; and U.S. Pat. Nos. 6,090,382; 6,258,562; and 6,509,015).

In certain embodiments, the anti-TNFα agent can include or is golimumab (golimumab™), adalimumab (Humira™), infliximab (Remicade™), CDP571, CDP 870, or certolizumab pegol (Cimzia™). In certain embodiments, the anti-TNFα agent can be a TNFα inhibitor biosimilar. Examples of approved and late-phase TNFα inhibitor biosimilars include, but are not limited to, infliximab biosimilars such as Flixabi™ (SB2) from Samsung Bioepis, Inflectra® (CT-P13) from Celltrion/Pfizer, GS071 from Aprogen, Remsima™, PF-06438179 from Pfizer/Sandoz, NI-071 from Nichi-Iko Pharmaceutical Co., and ABP 710 from Amgen; adalimumab biosimilars such as Amgevita® (ABP 501) from Amgen and Exemptia™ from Zydus Cadila, BMO-2 or MYL-1401-A from Biocon/Mylan, CHS-1420 from Coherus, FKB327 from Kyowa Kirin, and BI 695501 from Boehringer Ingelheim;Solymbic®, SB5 from Samsung Bioepis, GP-2017 from Sandoz, ONS-3010 from Oncobiologics, M923 from Momenta, PF-06410293 from Pfizer, and etanercept biosimilars such as Erelzi™ from Sandoz/Novartis, Brenzys™ (SB4) from Samsung Bioepis, GP2015 from Sandoz, TuNEX® from Mycenax, LBEC0101 from LG Life, and CHS-0214 from Coherus.

In some embodiments of any of the methods described herein, the anti-TNFα agent is selected from the group consisting of: adalimumab, certolizumab, etanercept, golimumab, infliximabm, CDP571, and CDP 870.

In some embodiments, any of the antibodies or antigen-binding fragments described herein has a dissociation constant (K_{D}) of less than 1×10⁻⁵ M (e.g., less than 0.5×10⁻⁵ M, less than 1×10⁻⁶ M, less than 0.5×10⁻⁶ M, less than 1×10⁻⁷ M, less than 0.5×10⁻⁷ M, less than 1×10⁻⁸ M, less than 0.5×10⁻⁸ M, less than 1×10⁻⁹ M, less than 0.5×10⁻⁹ M, less than 1×10⁻¹⁰ M, less than 0.5×10⁻¹⁰ M, less than 1×1⁻¹¹ M, less than 0.5×10⁻¹¹ M, or less than 1×10⁻¹² M), e.g., as measured in phosphate buffered saline using surface plasmon resonance (SPR).

In some embodiments, any of the antibodies or antigen-binding fragments described herein has a K_{D} of about 1×10⁻¹² M to about 1×10⁻⁵ M, about 0.5×10⁻⁵ M, about 1×10⁻⁶ M, about 0.5×10⁻⁶ M, about 1×10⁻⁷ M, about 0.5×10⁻⁷ M, about 1×10⁻⁸ M, about 0.5×10⁻⁸ M, about 1×10⁻⁹ M, about 0.5×10⁻⁹ M, about 1×10⁻¹⁰ M, about 0.5×10⁻¹⁰ M, about 1×10⁻¹¹ M, or about 0.5×10⁻¹¹ M (inclusive); about 0.5×10⁻¹¹ M to about 1×10⁻⁵ M, about 0.5×10⁻⁵ M, about 1×10⁻⁶ M, about 0.5×10⁻⁶ M, about 1×10⁻⁷ M, about 0.5×10⁻⁷ M, about 1×10⁻⁸ M, about 0.5×10⁻⁸ M, about 1×10⁻⁹ M, about 0.5×10⁻⁹ M, about 1×10⁻¹⁰ M, about 0.5×10⁻¹⁰ M, or about 1×10⁻¹¹ M (inclusive); about 1×10⁻¹¹ M to about 1×10⁻⁵ M, about 0.5×10⁻⁵ M, about 1×10⁻⁶ M, about 0.5×10⁻⁶ M, about 1×10⁻⁷ M, about 0.5×10⁻⁷ M, about 1×10⁻⁸ M, about 0.5×10⁻⁸ M, about 1×10⁻⁹ M, about 0.5×10⁻⁹ M, about 1×10⁻¹⁰ M, or about 0.5×10⁻¹⁰ M (inclusive); about 0.5×10⁻¹⁰ M to about 1×10⁻⁵ M, about 0.5×10⁻⁵ M, about 1×10⁻⁶ M, about 0.5×10⁻⁶ M, about 1×10⁻⁷ M, about 0.5×10⁻⁷ M, about 1×10⁻⁸ M, about 0.5×10⁻⁸ M, about 1×10⁻⁹ M, about 0.5×10⁻⁹ M, or about 1×10⁻¹⁰ M (inclusive); about 1×10⁻¹⁰ M to about 1×10⁻⁵ M, about 0.5×10⁻⁵ M, about 1×10⁻⁶ M, about 0.5×10⁻⁶ M, about 1×10⁻⁷ M, about 0.5×10⁻⁷ M, about 1×10⁻⁸ M, about 0.5×10⁻⁸ M, about 1×10⁻⁹ M, or about 0.5×10⁻⁹ M (inclusive); about 0.5×10⁻⁹ M to about 1×10⁻⁵ M, about 0.5×10⁻⁵ M, about 1×10⁻⁶ M, about 0.5×10⁻⁶ M, about 1×10⁻⁷ M, about 0.5×10⁻⁷ M, about 1×10⁻⁸ M, about 0.5×10⁻⁸ M, or about 1×10⁻⁹ M (inclusive); about 1×10⁻⁹ M to about 1×10⁻⁵ M, about 0.5×10⁻⁵ M, about 1×10⁻⁶ M, about 0.5×10⁻⁶ M, about 1×10⁻⁷ M, about 0.5×10⁻⁷ M, about 1×10⁻⁸ M, or about 0.5×10⁻⁸ M (inclusive); about 0.5×10⁻⁸ M to about 1×10⁻⁵ M, about 0.5×10⁻⁵ M, about 1×10⁻⁶ M, about 0.5×10⁻⁶ M, about 1×10⁻⁷ M, about 0.5×10⁻⁷ M, or about 1×10⁻⁸ M (inclusive); about 1×10⁻⁸ M to about 1×10⁻⁵ M, about 0.5×10⁻⁵ M, about 1×10⁻⁶ M, about 0.5×10⁻⁶ M, about 1×10⁻⁷ M, or about 0.5×10⁻⁷ M (inclusive); about 0.5×10⁻⁷ M to about 1×10⁻⁵ M, about 0.5×10⁻⁵ M, about 1×10⁻⁶ M, about 0.5×10⁻⁶ M, or about 1×10⁻⁷ M (inclusive); about 1×10⁻⁷ M to about 1×10⁻⁵ M, about 0.5×10⁻⁵ M, about 1×10⁻⁶ M, or about 0.5×10⁻⁶ M (inclusive); about 0.5×10⁻⁶ M to about 1×10⁻⁵ M, about 0.5×10⁻⁵ M, or about 1×10⁻⁶ M (inclusive); about 1×10⁻⁶ M to about 1×10⁻⁵ M or about 0.5×10⁻⁵ M (inclusive); or about 0.5×10⁻⁵ M to about 1×10⁻⁵ M (inclusive), e.g., as measured in phosphate buffered saline using surface plasmon resonance (SPR).

In some embodiments, any of the antibodies or antigen-binding fragments described herein has a K_{off} of about 1×10⁻⁶ s⁻¹ to about 1×10⁻³ s⁻¹, about 0.5×10⁻³ s⁻¹, about 1×10⁻⁴ s⁻¹, about 0.5×10⁻⁴ s⁻¹, about 1×10⁻⁵ s⁻¹, or about 0.5×10⁻⁵ s⁻¹ (inclusive); about 0.5×10⁻⁵ s⁻¹ to about 1×10⁻³ s⁻¹, about 0.5×10⁻³ s⁻¹, about 1×10⁻⁴ s⁻¹, about 0.5×10⁻⁴ s⁻¹, or about 1×10⁻⁵ s⁻¹ (inclusive); about 1×10⁻⁵ s⁻¹ to about 1×10⁻³ s⁻¹, about 0.5×10⁻³ s⁻¹, about 1×10⁻⁴ s⁻¹, or about 0.5×10⁻⁴ s⁻¹ (inclusive); about 0.5×10⁻⁴ s⁻¹ to about 1×10⁻³ s⁻¹, about 0.5×10⁻³ s⁻¹, or about 1×10⁻⁴ s⁻¹ (inclusive); about 1×10⁻⁴ s⁻¹ to about 1×10⁻³ s⁻¹, or about 0.5×10⁻³ s⁻¹ (inclusive); or about 0.5×10⁻⁵ s⁻¹ to about 1×10⁻³ s⁻¹ (inclusive), e.g., as measured in phosphate buffered saline using surface plasmon resonance (SPR).

In some embodiments, any of the antibodies or antigen-binding fragments described herein has a Kₒₙ of about 1×10² M⁻¹s⁻¹ to about 1×10⁶ M⁻¹s⁻¹, about 0.5×10⁶ M⁻¹s⁻¹, about 1×10⁵ M⁻¹s⁻¹, about 0.5×10⁵ M⁻¹s⁻¹, about 1×10⁴ M⁻¹s⁻¹, about 0.5×10⁴ M⁻¹s⁻¹, about 1×10³ M⁻¹s⁻¹, or about 0.5×10³ M⁻¹s⁻¹ (inclusive); about 0.5×10³ M⁻¹s⁻¹ to about 1×10⁶ M⁻¹s⁻¹, about 0.5×10⁶ M⁻¹s⁻¹, about 1×10⁵ M⁻¹s⁻¹, about 0.5×10⁵ M⁻¹s⁻¹, about 1×10⁴ M⁻¹s⁻¹, about 0.5×10⁴ M⁻¹s⁻¹, or about 1×10³ M⁻¹s⁻¹ (inclusive); about 1×10³M⁻¹s⁻¹ to about 1×10⁶ M⁻¹s⁻¹, about 0.5×10⁶ M⁻¹s⁻¹, about 1×10⁵ M⁻¹s⁻¹, about 0.5×10⁵ M⁻¹s⁻¹, about 1×10⁴ M⁻¹s⁻¹, or about 0.5×10⁴ M⁻¹s⁻¹ (inclusive); about 0.5×10⁴ M⁻¹s⁻¹ to about 1×10⁶ M⁻¹s⁻¹, about 0.5×10⁶ M⁻¹s⁻¹, about 1×10⁵ M⁻¹s⁻¹, about 0.5×10⁵ M⁻¹s⁻¹, or about 1×10⁴ M⁻¹s⁻¹ (inclusive); about 1×10⁴ M⁻¹s⁻¹ to about 1×10⁶ M⁻¹s⁻¹, about 0.5×10⁶ M⁻¹s⁻¹, about 1×10⁵ M⁻¹s⁻¹, or about 0.5×10⁵ M⁻¹s⁻¹ (inclusive); about 0.5×10⁵ M⁻¹s⁻¹ to about 1×10⁶ M⁻¹s⁻¹, about 0.5×10⁶ M⁻¹s⁻¹, or about 1×10⁵ M⁻¹s⁻¹ (inclusive); about 1×10⁵ M⁻¹s⁻¹ to about 1×10⁶ M⁻¹s⁻¹, or about 0.5×10⁶ M⁻¹s⁻¹ (inclusive); or about 0.5×10⁶ M⁻¹s⁻¹ to about 1×10⁶ M⁻¹s⁻¹ (inclusive), e.g., as measured in phosphate buffered saline using surface plasmon resonance (SPR).

### Fusion Proteins

In some embodiments, the anti-TNFα agent is a fusion protein (e.g., an extracellular domain of a TNFR fused to a partner peptide, e.g., an Fc region of an immunoglobulin, e.g., human IgG) (see, e.g., Deeg et al., Leukemia 16(2):162, 2002; Peppel et al., J. Exp. Med. 174(6):1483-1489, 1991) or a soluble TNFR (e.g., TNFR1 or TNFR2) that binds specifically to TNFα. In some embodiments, the anti-TNFα agent includes or is a soluble TNFα receptor (e.g., Bjornberg et al., Lymphokine Cytokine Res. 13(3):203-211, 1994; Kozak et al., Am. J. Physiol. Reg. Integrative Comparative Physiol. 269(1):R23-R29, 1995; Tsao et al., Eur Respir J. 14(3):490-495, 1999; Watt et al., J Leukoc Biol. 66(6):1005-1013, 1999; Mohler et al., J. Immunol. 151(3):1548-1561, 1993; Nophar et al., EMBO J. 9(10):3269, 1990; Piguet et al., Eur. Respiratory J. 7(3):515-518, 1994; and Gray et al., Proc. Natl. Acad. Sci. U.S.A. 87(19):7380-7384, 1990). In some embodiments, the anti-TNFα agent includes or is etanercept (Enbrel™) (see, e.g., WO 91/03553 and WO 09/406,476, incorporated by reference herein). In some embodiments, the anti-TNFα agent inhibitor includes or is r-TBP-I (e.g., Gradstein et al., J. Acquir. Immune Defic. Syndr. 26(2): 111-117, 2001).

### Inhibitory Nucleic Acids

Inhibitory nucleic acids that can decrease the expression of AP-1, ASK1, CD14, c-jun, ERK1/2, IκB, IKK, IRAK, JNK, LBP, MAPK, MEK1/2, MEKK1/4, MEKK4/7, MEKK 3/6, MK2, MyD88, NF-κB, NIK, p38, PKR, rac, ras, raf, RIP, TNFα, TNFR1, TNFR2, TRADD, TRAF2, TRAF6, or TTP mRNA expression in a mammalian cell include antisense nucleic acid molecules, i.e., nucleic acid molecules whose nucleotide sequence is complementary to all or part of a AP-1, ASK1, CD14, c-jun, ERK1/2, IκB, IKK, IRAK, JNK, LBP, MAPK, MEK1/2, MEKK1/4, MEKK4/7, MEKK 3/6, MK2, MyD88, NF-κB, NIK, p38, PKR, rac, ras, raf, RIP, TNFα, TNFR1, TNFR2, TRADD, TRAF2, TRAF6, or TTP mRNA (e.g fully or partially complementary to all or a part of any one of the sequences presented in table E).

| | mRNA GenBank |
|---|---|
| Human gene | accession number(s) |
| Tumor necrosis factor | NM_000594 |
| (TNF, a.k.a. TNF-alpha) | |
| TNF receptor superfamily member 1A | NM_001065 |
| (TNFRSF1A) (a.k.a. TNFR1) | NM_001346091 |
| | NM_001346092 |
| TNF receptor superfamily member 1B | NM_001066 |
| (TNFRSF1B) (a.k.a. TNFR2) | XM_011542060 |
| | XM_011542063 |
| | XM_017002214 |
| | XM_017002215 |
| | XM_017002211 |
| TNFRSF1A associated via death domain | NM_003789 |
| (TRADD) | NM_001323552 |
| | XM_005256213 |
| | XM_017023815 |
| TNF receptor associated factor 2 (TRAF2) | NM_021138 |
| | XM_011518976 |
| | XM_011518977 |
| | XM_011518974 |
| JunD proto-oncogene, AP-1 transcription | NM_001286968 |
| factor subunit (JUND) | NM_005354 |
| Mitogen-activated protein kinase kinase | NM_005923 |
| kinase 5 (MAP3K5) (a.k.a. ASK1) | XM_017010875 |
| | XM_017010872 |
| | XM_017010873 |
| | XM_017010877 |
| | XM_017010874 |
| | XM_017010871 |
| | XM_017010870 |
| | XM_017010876 |
| | XM_011535839 |
| CD14 | NM_000591 |
| | NM_001040021 |
| | NM_001174104 |
| | NM_001174105 |
| Mitogen-activated protein kinase 3 | NM_001040056 |
| (MAPK3) (a.k.a. ERK1) | NM_001109891 |
| | NM_002746 |
| Mitogen-activated protein kinase 1 | NM_002745 |
| (MAPK1) (a.k.a. ERK2) | NM_138957 |
| Inhibitor of nuclear factor kappa B kinase | NM_001190720 |
| subunit beta (IKBKB) | NM_001242778 |
| | NM_001556 |
| | XM_005273491 |
| | XM_005273496 |
| | XM_005273493 |
| | XM_005273498 |
| | XM_011544518 |
| | XM_005273492 |
| | XM_005273490 |
| | XM_005273494 |
| | 12XM_017013396 |
| | XM_011544521 |
| | XM_011544522 |
| | XM_005273495 |
| | XM_011544517 |
| | XM_011544520 |
| | XM_011544519 |
| NFKB inhibitor alpha (NFKBIA) | NM_020529 |
| Interleukin 1 receptor associated kinase 1 | NM_001025242 |
| (IRAK1) | NM_001025243 |
| | NM_001569 |
| | XM_005274668 |
| Mitogen-activated protein kinase 8 | NM_001278547 |
| (MAPK8) (a.k.a. JNK) | NM_001278548 |
| | NM_001323302 |
| | NM_001323320 |
| | NM_001323321 |
| | NM_001323322 |
| | NM_001323323 |
| | NM_001323324 |
| | NM_001323325 |
| | NM_001323326 |
| | NM_001323327 |
| | NM_001323328 |
| | NM_001323329 |
| | NM_001323330 |
| | NM_001323331 |
| | NM_139046 |
| | NM_139049 |
| | XM_024448079 |
| | XM_024448080 |
| Lipopolysaccharide binding protein (LBP) | NM_004139 |
| Mitogen-activated protein kinase kinase 1 | NM_002755 |
| (MAP2K1) (a.k.a. MEK1) | XM_017022411 |
| | XM_011521783 |
| | XM_017022412 |
| | XM_017022413 |
| Mitogen-activated protein kinase kinase 2 | NM_030662 |
| (MAP2K2) (a.k.a. MEK2) | XM_006722799 |
| | XM_017026990 |
| | XM_017026989 |
| | XM_017026991 |
| Mitogen-activated protein kinase kinase 3 | NM_001316332 |
| (MAP2K3) (a.k.a. MEK3) | NM_002756 |
| | NM_145109 |
| | XM_017024859 |
| | XM_005256723 |
| | XM_017024857 |
| | XM_011523959 |
| | XM_017024858 |
| | XM_011523958 |
| Mitogen-activated protein kinase kinase 6 | NM_001330450 |
| (MAP2K6) (a.k.a. MEK6) | NM_002758 |
| | XM_005257516 |
| | XM_011525027 |
| | XM_011525026 |
| | XM_006721975 |
| Mitogen-activated protein kinase kinase | NM_005921 |
| kinase 1 (MAP3K1) (a.k.a. MEKK1) | XM_017009485 |
| | XM_017009484 |
| Mitogen-activated protein kinase kinase | NM_001330431 |
| kinase 3 (MAP3K3) (a.k.a. MEKK3) | NM_001363768 |
| | NM_002401 |
| | NM_203351 |
| | XM_005257378 |
| Mitogen-activated protein kinase kinase | NM_001291958 |
| kinase 4 (MAP3K4) (a.k.a. MEKK4) | NM_001301072 |
| | NM_001363582 |
| | NM_005922 |
| | NM_006724 |
| | XM_017010869 |
| Mitogen-activated protein kinase kinase | NM_001297609 |
| kinase 6 (MAP3K6) (a.k.a. MEKK6) | NM_004672 |
| | XM_017002771 |
| | XM_017002772 |
| Mitogen-activated protein kinase kinase | NM_003188 |
| kinase 7 (MAP3K7) (a.k.a. MEKK7) | NM_145331 |
| | NM_145332 |
| | NM_145333 |
| | XM_006715553 |
| | XM_017011226 |
| MAPK activated protein kinase 2 | NM_004759 |
| (MAPKAPK2) (a.k.a. MK2) | NM_032960 |
| | XM_005273353 |
| | XM_017002810 |
| MYD88, innate immune signal transduction | NM_001172566 |
| adaptor (MYD88) | NM_001172567 |
| | NM_001172568 |
| | NM_001172569 |
| | NM_001365876 |
| | NM_001365877 |
| | NM_002468 |
| Nuclear factor kappa B subunit 1 (NFKB1) | NM_001165412 |
| | NM_001319226 |
| | NM_003998 |
| | XM_024454069 |
| | XM_024454067 |
| | XM_011532006 |
| | XM_024454068 |
| Mitogen-activated protein kinase kinase | NM_003954 |
| kinase 14 (MAP3K14) (a.k.a. NIK) | XM_011525441 |
| Mitogen-activated protein kinase 14 | NM_001315 |
| (MAPK14) (a.k.a. p38) | NM_139012 |
| | NM_139013 |
| | NM_139014 |
| | XM_011514310 |
| | XM_017010300 |
| | XM_017010299 |
| | XM_017010301 |
| | XM_017010304 |
| | XM_017010303 |
| | XM_017010302 |
| | XM_006714998 |
| Eukaryotic translation initiation factor 2 | NM_001135651 |
| alpha kinase 2 (EIF2AK2) (a.k.a. PKR) | NM_001135652 |
| | NM_002759 |
| | XM_011532987 |
| | XM_017004503 |
| AKT serine/threonine kinase 1 (AKT1) | NM_001014431 |
| (a.k.a. RAC) | NM_001014432 |
| | NM_005163 |
| Zinc fingers and homeoboxes 2 (ZHX2) | NM_001362797 |
| (a.k.a. RAF) | NM_014943 |
| | XM_011516932 |
| | XM_005250836 |
| KRAS proto-oncogene, GTPase (KRAS) | NM_001369786 |
| | NM_001369787 |
| | NM_004985 |
| | NM_033360 |
| NRAS proto-oncogene, GTPase (NRAS) | NM_002524 |
| Receptor interacting serine/threonine kinase | NM_001317061 |
| 1 (RIPK1) (a.k.a. RIP) | NM_001354930 |
| | NM_001354931 |
| | NM_001354932 |
| | NM_001354933 |
| | NM_001354934 |
| | NM_003804 |
| | XM_017011405 |
| | XM_006715237 |
| | XM_017011403 |
| | XM_017011404 |
| TNF receptor associated factor 6 (TRAF6) | NM_004620 |
| | NM_145803 |
| | XM_017018220 |
| ZFP36 ring finger protein (ZFP36) (a.k.a. | NM_003407 |
| TTP) | |

An antisense nucleic acid molecule can be complementary to all or part of a non-coding region of the coding strand of a nucleotide sequence encoding an AP-1, ASK1, CD14, c-jun, ERK1/2, IκB, IKK, IRAK, JNK, LBP, MAPK, MEK1/2, MEKK1/4, MEKK4/7, MEKK 3/6, MK2, MyD88, NF-κB, NIK, p38, PKR, rac, ras, raf, RIP, TNFα, TNFR1, TNFR2, TRADD, TRAF2, TRAF6, or TTPMEKK1protein. Non-coding regions (5′ and 3′ untranslated regions) are the 5′ and 3′ sequences that flank the coding region in a gene and are not translated into amino acids.

Based upon the sequences disclosed herein, one of skill in the art can easily choose and synthesize any of a number of appropriate antisense nucleic acids to target a nucleic acid encoding an AP-1, ASK1, CD14, c-jun, ERK1/2, IκB, IKK, IRAK, JNK, LBP, MAPK, MEK1/2, MEKK1/4, MEKK4/7, MEKK 3/6, MK2, MyD88, NF-κB, NIK, p38, PKR, rac, ras, raf, RIP, TNFα, TNFR1, TNFR2, TRADD, TRAF2, TRAF6, or TTP protein described herein. Antisense nucleic acids targeting a nucleic acid encoding an AP-1, ASK1, CD14, c-jun, ERK1/2, IκB, IKK, IRAK, JNK, LBP, MAPK, MEK1/2, MEKK1/4, MEKK4/7, MEKK 3/6, MK2, MyD88, NF-κB, NIK, p38, PKR, rac, ras, raf, RIP, TNFα, TNFR1, TNFR2, TRADD, TRAF2, TRAF6, or TTPMEKK1protein can be designed using the software available at the Integrated DNA Technologies website.

An antisense nucleic acid can be, for example, about 5, 10, 15, 18, 20, 22, 24, 25, 26, 28, 30, 32, 35, 36, 38, 40, 42, 44, 45, 46, 48, or 50 nucleotides or more in length. An antisense oligonucleotide can be constructed using enzymatic ligation reactions and chemical synthesis using procedures known in the art. For example, an antisense nucleic acid can be chemically synthesized using variously modified nucleotides or naturally occurring nucleotides designed to increase the physical stability of the duplex formed between the antisense and sense nucleic acids, e.g., phosphorothioate derivatives and acridine substituted nucleotides or to increase the biological stability of the molecules.

Examples of modified nucleotides which can be used to generate an antisense nucleic acid include 1-methylguanine, 1-methylinosine, 2,2-dimethylguanine, 2-methyladenine, 2-methylguanine, 3-methylcytosine, 2-methylthio-N6-i sopentenyladenine, uracil-5-oxyacetic acid (v), wybutoxosine, pseudouracil, queosine, 2-thiocytosine, 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an anti sense orientation (i.e., RNA transcribed from the inserted nucleic acid will be of an antisense orientation to a target nucleic acid of interest).

The antisense nucleic acid molecules described herein can be prepared in vitro and administered to a subject, e.g., a human subject. Alternatively, they can be generated in situ such that they hybridize with or bind to cellular mRNA and/or genomic DNA encoding an AP-1, ASK1, CD14, c-jun, ERK1/2, IκB, IKK, IRAK, JNK, LBP, MAPK, MEK1/2, MEKK1/4, MEKK4/7, MEKK 3/6, MK2, MyD88, NF-κB, NIK, p38, PKR, rac, ras, raf, RIP, TNFα, TNFR1, TNFR2, TRADD, TRAF2, TRAF6, or TTP protein to thereby inhibit expression, e.g., by inhibiting transcription and/or translation. The hybridization can be by conventional nucleotide complementarities to form a stable duplex, or, for example, in the case of an antisense nucleic acid molecule that binds to DNA duplexes, through specific interactions in the major groove of the double helix. The antisense nucleic acid molecules can be delivered to a mammalian cell using a vector (e.g., an adenovirus vector, a lentivirus, or a retrovirus).

An antisense nucleic acid can be an α-anomeric nucleic acid molecule. An a-anomeric nucleic acid molecule forms specific double-stranded hybrids with complementary RNA in which, contrary to the usual, β-units, the strands run parallel to each other (Gaultier et al., Nucleic Acids Res. 15:6625-6641, 1987). The antisense nucleic acid can also comprise a chimeric RNA-DNA analog (Inoue et al., FEBS Lett. 215:327-330, 1987) or a 2′-O-methylribonucleotide (Inoue et al., Nucleic Acids Res. 15:6131-6148, 1987).

Another example of an inhibitory nucleic acid is a ribozyme that has specificity for a nucleic acid encoding an AP-1, ASK1, CD14, c-jun, ERK1/2, IκB, IKK, IRAK, JNK, LBP, MAPK, MEK1/2, MEKK1/4, MEKK4/7, MEKK 3/6, MK2, MyD88, NF-κB, NIK, p38, PKR, rac, ras, raf, RIP, TNFα, TNFR1, TNFR2, TRADD, TRAF2, TRAF6, or TTP mRNA, e.g., e.g fully or partially complementary to all or a part of any one of the sequences presented in table E). Ribozymes are catalytic RNA molecules with ribonuclease activity that are capable of cleaving a single-stranded nucleic acid, such as an mRNA, to which they have a complementary region. Thus, ribozymes (e.g., hammerhead ribozymes (described in Haselhoff and Gerlach, Nature 334:585-591, 1988)) can be used to catalytically cleave mRNA transcripts to thereby inhibit translation of the protein encoded by the mRNA. An AP-1, ASK1, CD14, c-jun, ERK1/2, IκB, IKK, IRAK, JNK, LBP, MAPK, MEK1/2, MEKK1/4, MEKK4/7, MEKK 3/6, MK2, MyD88, NF-κB p38, PKR, rac, ras, raf, RIP, TNFα, TNFR1, TNFR2, TRADD, TRAF2, TRAF6, or TTP mRNA can be used to select a catalytic RNA having a specific ribonuclease activity from a pool of RNA molecules. See, e.g., Bartel et al., Science 261:1411-1418, 1993.

Alternatively, a ribozyme having specificity for an AP-1, ASK1, CD14, c-jun, ERK1/2, IκB, IKK, IRAK, JNK, LBP, MAPK, MEK1/2, MEKK1/4, MEKK4/7, MEKK 3/6, MK2, MyD88, NF-κB, NIK, p38, PKR, rac, ras, raf, RIP, TNFα, TNFR1, TNFR2, TRADD, TRAF2, TRAF6, or TTP mRNA can be designed based upon the nucleotide sequence of any of the AP-1, ASK1, CD14, c-jun, ERK1/2, IκB, IKK, IRAK, JNK, LBP, MAPK, MEK1/2, MEKK1/4, MEKK4/7, MEKK 3/6, MK2, MyD88, NF-κB, NIK, p38, PKR, rac, ras, raf, RIP, TNFα, TNFR1, TNFR2, TRADD, TRAF2, TRAF6, or TTP mRNA sequences disclosed herein. For example, a derivative of a Tetrahymena L-19 IVS RNA can be constructed in which the nucleotide sequence of the active site is complementary to the nucleotide sequence to be cleaved in an AP-1, ASK1, CD14, c-jun, ERK1/2, IκB, IKK, IRAK, JNK, LBP, MAPK, MEK1/2, MEKK1/4, MEKK4/7, MEKK 3/6, MK2, MyD88, NF-κB, NIK, p38, PKR, rac, ras, raf, RIP, TNFα, TNFR1, TNFR2, TRADD, TRAF2, TRAF6, or TTP mRNA (see, e.g., U.S. Pat. . Nos. 4,987,071 and 5,116,742).

An inhibitory nucleic acid can also be a nucleic acid molecule that forms triple helical structures. For example, expression of an AP-1, ASK1, CD14, c-jun, ERK1/2, IκB, IKK, IRAK, JNK, LBP, MAPK, MEK1/2, MEKK1/4, MEKK4/7, MEKK 3/6, MK2, MyD88, NF-κB, NIK, p38, PKR, rac, ras, raf, RIP, TNFα, TNFR1, TNFR2, TRADD, TRAF2, TRAF6, or TTP polypeptide can be inhibited by targeting nucleotide sequences complementary to the regulatory region of the gene encoding the AP-1, ASK1, CD14, c-jun, ERK1/2, IκB, IKK, IRAK, JNK, LBP, MAPK, MEK1/2, MEKK1/4, MEKK4/7, MEKK 3/6, MK2, MyD88, NF-κB, NIK, p38, PKR, rac, ras, raf, RIP, TNFα, TNFR1, TNFR2, TRADD, TRAF2, TRAF6, or TTP polypeptide (e.g., the promoter and/or enhancer, e.g., a sequence that is at least 1 kb, 2 kb, 3 kb, 4 kb, or 5 kb upstream of the transcription initiation start state) to form triple helical structures that prevent transcription of the gene in target cells. See generally Maher, Bioassays 14(12):807-15, 1992; Helene, Anticancer Drug Des. 6(6):569-84, 1991; and Helene, Ann. N.Y. Acad. Sci. 660:27-36, 1992.

In various embodiments, inhibitory nucleic acids can be modified at the sugar moiety, the base moiety, or phosphate backbone to improve, e.g., the solubility, stability, or hybridization, of the molecule. For example, the deoxyribose phosphate backbone of the nucleic acids can be modified to generate peptide nucleic acids (see, e.g., Hyrup et al., Bioorganic Medicinal Chem. 4(1):5-23, 1996). Peptide nucleic acids (PNAs) are nucleic acid mimics, e.g., DNA mimics, in which the deoxyribose phosphate backbone is replaced by a pseudopeptide backbone and only the four natural nucleobases are retained. The neutral backbone of PNAs allows for specific hybridization to RNA and DNA under conditions of low ionic strength. PNA oligomers can be synthesized using standard solid phase peptide synthesis protocols (see, e.g., Perry-O'Keefe et al., Proc. Natl. Acad. Sci. U.S.A. 93:14670-675, 1996). PNAs can be used as antisense or antigene agents for sequence-specific modulation of gene expression by, e.g., inducing transcription or translation arrest or inhibiting replication.

### Small Molecules

In some embodiments, the anti-TNFα agent is a small molecule. In some embodiments, the small molecule is a tumor necrosis factor-converting enzyme (TACE) inhibitor (e.g., Moss et al., Nature Clinical Practice Rheumatology 4: 300-309, 2008). In some embodiments, the anti-TNFα agent is C87 (Ma et al., J. Biol. Chem. 289(18):12457-66, 2014). In some embodiments, the small molecule is LMP-420 (e.g., Haraguchi et al., AIDS Res. Ther. 3:8, 2006). In some embodiments, the TACE inhibitor is TMI-005 and BMS-561392. Additional examples of small molecule inhibitors are described in, e.g., He et al., Science 310(5750):1022-1025, 2005.

In some examples, the anti-TNFα agent is a small molecule that inhibits the activity of one of AP-1, ASK1, IKK, JNK, MAPK, MEKK 1/4, MEKK4/7, MEKK 3/6, NIK, TRADD, RIP, NF-κB, and TRADD in a cell (e.g., in a cell obtained from a subject, a mammalian cell).

In some examples, the anti-TNFα agent is a small molecule that inhibits the activity of one of CD14, MyD88 (see, e.g., Olson et al., Scientific Reports 5:14246, 2015), ras (e.g., Baker et al., Nature 497:577-578, 2013), raf (e.g., vemurafenib (PLX4032, RG7204), sorafenib tosylate, PLX-4720, dabrafenib (GSK2118436), GDC-0879, RAF265 (CHIR-265), AZ 628, NVP-BHG712, SB590885, ZM 336372, sorafenib, GW5074, TAK-632, CEP-32496, encorafenib (LGX818), CCT196969, LY3009120, RO5126766 (CH5126766), PLX7904, and MLN2480).

In some examples, the anti-TNFα agent TNFα inhibitor is a small molecule that inhibits the activity of one of MK2 (PF 3644022 and PHA 767491), JNK (e.g., AEG 3482, BI 78D3, CEP 1347, c-JUN peptide, IQ 1S, JIP-1 (153-163), SP600125, SU 3327, and TCS JNK6o), c-jun (e.g., AEG 3482, BI 78D3, CEP 1347, c-JUN peptide, IQ 1S, JIP-1 (153-163), SP600125, SU 3327, and TCS JNK6o), MEK3/6 (e.g., Akinleye et al., J. Hematol. Oncol. 6:27, 2013), p38 (e.g., AL 8697, AMG 548, BIRB 796, CMPD-1, DBM 1285 dihydrochloride, EO 1428, JX 401, ML 3403, Org 48762-0, PH 797804, RWJ 67657, SB 202190, SB 203580, SB 239063, SB 706504, SCIO 469, SKF 86002, SX 011, TA 01, TA 02, TAK 715, VX 702, and VX 745), PKR (e.g., 2-aminopurine or CAS 608512-97-6), TTP (e.g., CAS 329907-28-0), MEK1/2 (e.g., Facciorusso et al., Expert Review Gastroentrol. Hepatol. 9:993-1003, 2015), ERK1/2 (e.g., Mandal et al., Oncogene 35:2547-2561, 2016), NIK (e.g., Mortier et al., Bioorg. Med. Chem. Lett. 20:4515-4520, 2010), IKK (e.g., Reilly et al., Nature Med. 19:313-321, 2013), I⁻KB (e.g., Suzuki et al., Expert. Opin. Invest. Drugs 20:395-405, 2011), NF-κB (e.g., Gupta et al., Biochim. Biophys. Acta 1799(10-12):775-787, 2010), rac (e.g., U.S. Pat. No. 9,278,956), MEK4/7, IRAK (Chaudhary et al., J. Med. Chem. 58(1):96-110, 2015), LBP (see, e.g., U.S. Pat. No. 5,705,398), and TRAF6 (e.g., 3-[(2,5-Dimethylphenyl)amino]-1-phenyl-2-propen-1-one).

In some embodiments of any of the methods described herein, the inhibitory nucleic acid can be about 10 nucleotides to about 50 nucleotides (e.g., about 10 nucleotides to about 45 nucleotides, about 10 nucleotides to about 40 nucleotides, about 10 nucleotides to about 35 nucleotides, about 10 nucleotides to about 30 nucleotides, about 10 nucleotides to about 28 nucleotides, about 10 nucleotides to about 26 nucleotides, about 10 nucleotides to about 25 nucleotides, about 10 nucleotides to about 24 nucleotides, about 10 nucleotides to about 22 nucleotides, about 10 nucleotides to about 20 nucleotides, about 10 nucleotides to about 18 nucleotides, about 10 nucleotides to about 16 nucleotides, about 10 nucleotides to about 14 nucleotides, about 10 nucleotides to about 12 nucleotides, about 12 nucleotides to about 50 nucleotides, about 12 nucleotides to about 45 nucleotides, about 12 nucleotides to about 40 nucleotides, about 12 nucleotides to about 35 nucleotides, about 12 nucleotides to about 30 nucleotides, about 12 nucleotides to about 28 nucleotides, about 12 nucleotides to about 26 nucleotides, about 12 nucleotides to about 25 nucleotides, about 12 nucleotides to about 24 nucleotides, about 12 nucleotides to about 22 nucleotides, about 12 nucleotides to about 20 nucleotides, about 12 nucleotides to about 18 nucleotides, about 12 nucleotides to about 16 nucleotides, about 12 nucleotides to about 14 nucleotides, about 15 nucleotides to about 50 nucleotides, about 15nucleotides to about 45 nucleotides, about 15nucleotides to about 40 nucleotides, about 15nucleotides to about 35 nucleotides, about 15 nucleotides to about 30 nucleotides, about 15nucleotides to about 28 nucleotides, about 15nucleotides to about 26 nucleotides, about 15nucleotides to about 25 nucleotides, about 15nucleotides to about 24 nucleotides, about 15nucleotides to about 22 nucleotides, about 15nucleotides to about 20 nucleotides, about 15nucleotides to about 18 nucleotides, about 15nucleotides to about 16 nucleotides, about 16 nucleotides to about 50 nucleotides, about 16 nucleotides to about 45 nucleotides, about 16 nucleotides to about 40 nucleotides, about 16 nucleotides to about 35 nucleotides, about 16 nucleotides to about 30 nucleotides, about 16 nucleotides to about 28 nucleotides, about 16 nucleotides to about 26 nucleotides, about 16 nucleotides to about 25 nucleotides, about 16 nucleotides to about 24 nucleotides, about 16 nucleotides to about 22 nucleotides, about 16 nucleotides to about 20 nucleotides, about 16 nucleotides to about 18 nucleotides, about 18 nucleotides to about 20 nucleotides, about 20 nucleotides to about 50 nucleotides, about 20 nucleotides to about 45 nucleotides, about 20 nucleotides to about 40 nucleotides, about 20 nucleotides to about 35 nucleotides, about 20 nucleotides to about 30 nucleotides, about 20 nucleotides to about 28 nucleotides, about 20 nucleotides to about 26 nucleotides, about 20 nucleotides to about 25 nucleotides, about 20 nucleotides to about 24 nucleotides, about 20 nucleotides to about 22 nucleotides, about 24 nucleotides to about 50 nucleotides, about 24 nucleotides to about 45 nucleotides, about 24 nucleotides to about 40 nucleotides, about 24 nucleotides to about 35 nucleotides, about 24 nucleotides to about 30 nucleotides, about 24 nucleotides to about 28 nucleotides, about 24 nucleotides to about 26 nucleotides, about 24 nucleotides to about 25 nucleotides, about 26 nucleotides to about 50 nucleotides, about 26 nucleotides to about 45 nucleotides, about 26 nucleotides to about 40 nucleotides, about 26 nucleotides to about 35 nucleotides, about 26 nucleotides to about 30 nucleotides, about 26 nucleotides to about 28 nucleotides, about 28 nucleotides to about 50 nucleotides, about 28 nucleotides to about 45 nucleotides, about 28 nucleotides to about 40 nucleotides, about 28 nucleotides to about 35 nucleotides, about 28 nucleotides to about 30 nucleotides, about 30 nucleotides to about 50 nucleotides, about 30 nucleotides to about 45 nucleotides, about 30 nucleotides to about 40 nucleotides, about 30 nucleotides to about 38 nucleotides, about 30 nucleotides to about 36 nucleotides, about 30 nucleotides to about 34 nucleotides, about 30 nucleotides to about 32 nucleotides, about 32 nucleotides to about 50 nucleotides, about 32 nucleotides to about 45 nucleotides, about 32 nucleotides to about 40 nucleotides, about 32 nucleotides to about 35 nucleotides, about 35 nucleotides to about 50 nucleotides, about 35 nucleotides to about 45 nucleotides, about 35 nucleotides to about 40 nucleotides, about 40 nucleotides to about 50 nucleotides, about 40 nucleotides to about 45 nucleotides, about 42 nucleotides to about 50 nucleotides, about 42 nucleotides to about 45 nucleotides, or about 45 nucleotides to about 50 nucleotides) in length. One skilled in the art will appreciate that inhibitory nucleic acids may comprises at least one modified nucleic acid at either the 5′ or 3′ end of DNA or RNA.

In some embodiments, the inhibitory nucleic acid can be formulated in a liposome, a micelle (e.g., a mixed micelle), a nanoemulsion, or a microemulsion, a solid nanoparticle, or a nanoparticle (e.g., a nanoparticle including one or more synthetic polymers). Additional exemplary structural features of inhibitory nucleic acids and formulations of inhibitory nucleic acids are described in US 2016/0090598.

In some embodiments, the inhibitory nucleic acid (e.g., any of the inhibitory nucleic acid described herein) can include a sterile saline solution (e.g., phosphate-buffered saline (PBS)). In some embodiments, the inhibitory nucleic acid (e.g., any of the inhibitory nucleic acid described herein) can include a tissue-specific delivery molecule (e.g., a tissue-specific antibody).

Compound Preparation and Biological Assays

As can be appreciated by the skilled artisan, methods of synthesizing the compounds of the formulae herein will be evident to those of ordinary skill in the art. Synthetic chemistry transformations and protecting group methodologies (protection and deprotection) useful in synthesizing the compounds described herein are known in the art and include, for example, those such as described in R. Larock, Comprehensive Organic Transformations, VCH Publishers (1989); T. W. Greene and RGM. Wuts, Protective Groups in Organic Synthesis, 2d. Ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

### PREPARATIVE EXAMPLES

The following abbreviations have the indicated meanings:
BTC=trichloromethyl chloroformate
Boc=t-butyloxy carbonyl
DCM=dichloromethane
DMF=N,N-dimethylformamide
DMSO=dimethyl sulfoxide
DIEA=N,N-diisopropylethylamine
EtOH=ethanol
EtOAc=ethyl acetate
Hex=hexane
HPLC=high performance liquid chromatography
LC-MS=liquid chromatography-mass spectrometry
LiHMDS=lithium bis(trimethylsilyl)amide
LDA=lithium diisopropylamide
MeCN=acetonitrile
Me=methyl
MeOH=methanol
MSA=methanesulfonic acid
NCS=N-chlorosuccinimide
NIS=N-iodosuccinimide
NMR=nuclear magnetic resonance
Pd2(dba)3=Tris(dibenzylideneacetone)dipalladium(0)
Pd(dppf)Cl2=1,1′-Bis(diphenylphosphino)ferrocene]dichloropalladium(II)
PE=petroleum ether
Ph=phenyl
PPh₃Cl2=dichlorotriphenylphosphorane
Py=pyridine
RT=room temperature
Rt=retention time
Sat.=saturated
TBS=tert-butyldimethylsilyl
TBSCl=tert-butyldimethylsilyl chloride
TEA=triethylamine
TFA=trifluoroacetic acid
THF=tetrahydrofuran
TLC=thin layer chromatography
TsOH=4-methylbenzenesulfonic acid
Xphos=2-dicyclohexylphosphino-2′,4′,6′-triisopropylbiphenyl
UV=ultraviolet

General

The progress of reactions was often monitored by TLC or LC-MS. The identity of the products was often confirmed by LC-MS. The LC-MS was recorded using one of the following methods.

Method A: Shim-pack XR-ODS, C18, 3×50 mm, 2.5 um column, 1.0 uL injection, 1.5 mL/min flow rate, 90-900 amu scan range, 190-400 nm UV range, 5-100% (1.1 min), 100% (0.6 min) gradient with MeCN (0.05% TFA) and water (0.05% TFA), 2 minute total run time.

Method B: Kinetex EVO, C18, 3×50 mm, 2.2 um column, 1.0 uL injection, 1.5 mL/min flow rate, 90-900 amu scan range, 190-400 nm UV range, 10-95% (1.1 min), 95% (0.6 min) gradient with MeCN and water (0.5% NH₄HCO3), 2 minute total run time.

Method C: Shim-pack XR-ODS, C18, 3×50 mm, 2.5 um column, 1.0 uL injection, 1.5 mL/min flow rate, 90-900 amu scan range, 190-400 nm UV range, 5-100% (2.1 min), 100% (0.6 min) gradient with MeCN (0.05% TFA) and water (0.05% TFA), 3 minute total run time.

Method D: Kinetex EVO, C18, 3×50 mm, 2.2 um column, 1.0 uL injection, 1.5 mL/min flow rate, 90-900 amu scan range, 190-400 nm UV range, 10-95% (2.1 min), 95% (0.6 min) gradient with MeCN and water (0.5% NH₄HCO3), 3 minute total run time.

Method F: Phenomenex, CHO-7644, Onyx Monolithic C18, 50×4.6 mm, 10.0 uL injection, 1.5 mL/min flow rate, 100-1500 amu scan range, 220 and 254 nm UV detection, 5% with MeCN (0.1% TFA) to 100% water (0.1% TFA) over 9.5 min, with a stay at 100% (MeCN, 0.1% TFA) for 1 min, then equilibration to 5% (MeCN, 0.1% TFA) over 1.5 min.

The final targets were purified by Prep-HPLC. The Prep-HPLC was carried out using the following method.

Method E: Prep-HPLC: XSelect CSH Prep C18 OBD Column, 19*150 mm; 5 um; mobile phase, Water water (10 mM NH₄HCO3+0.1% NH3.H₂O) and MeCN, UV detection 254/210 nm.

NMR was recorded on BRUKER NMR 300.03 MHz, DUL-C-H, ULTRASHIELDTM300, AVANCE II 300 B-ACSTM120 or BRUKER NMR 400.13 MHz, BBFO, ULTRASHIELDTM400, AVANCE III 400, B-ACSTM120.

Scheme of final targets: Schemes I illustrate the condition used for final product.

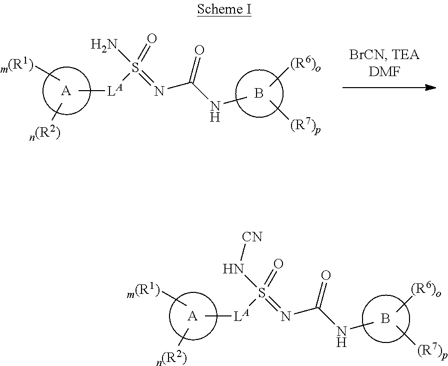

Schemes for Sulfonimidoylamide Intermediates: Schemes 1-5 illustrate the preparation of sulfonimidamide intermediates.

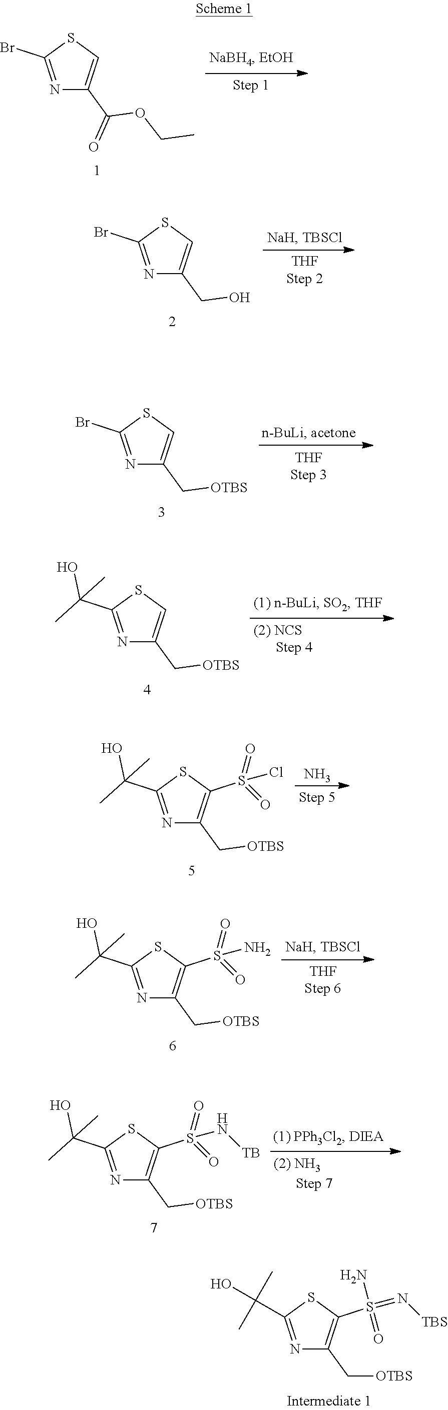

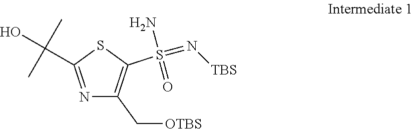

### N′-(tert-butyldimethylsilyl)-4-((tert-butyldimethylsilyloxy)methyl)-2-(2-hydroxypropan-2-yl)thiazole-5-sulfonimidamide

### Step 1: (2-Bromothiazol-4-yl)methanol

To a stirred solution of ethyl 2-bromo-1,3-thiazole-4-carboxylate (3.0 g, 12.7 mmol) in EtOH (30 mL) in a 100-mL round-bottom flask under nitrogen was added NaBH₄ (1.0 g, 25.4 mmol) in portions at 0° C. in an ice/water bath. The resulting solution was stirred for 3 h at RT. The reaction was then quenched by the addition of 100 mL of water in an ice/water bath. The resulting solution was extracted with 3×100 mL of EtOAc, the combined organic layers were dried over anhydrous Na₂SO₄ and concentrated. This resulted in 2.0 g (81%) of the title compound as yellow oil. MS-ESI: 196/194 (M+1).

### Step 2: 2-Bromo-4-((tert-butyldimethylsilyloxy)methyl)thiazole

To a stirred solution of (2-bromo-1,3-thiazol-4-yl)methanol (2.0 g, 10.3 mmol) in THF (20 mL) in a 100-mL round-bottom flask was added NaH (60% wt. oil dispersion, 1.2 g, 31 mmol) in portions at 0° C. in an ice/water bath. After stirring for 15 min at RT, a solution of TBSCl (4.7 g, 30.9 mmol) in THF (5 mL) was added to this reaction mixture dropwise at 0° C. The resulting solution was stirred for 2 h at RT. The reaction was then quenched by the addition of 50 mL of water. The resulting solution was extracted with 3×100 mL of EtOAc, the organic layers were combined, dried over anhydrous Na₂SO₄ and concentrated under vacuum. The residue was eluted from silica gel with EtOAc/PE (1:30). This resulted in 2.5 g (79%) of the title compound as yellow oil. MS-ESI: 310/308 (M+1).

### Step 3: 2-(4-((Tert-butyldimethylsilyloxy)methyl)thiazol-2-yl)propan-2-ol

To a stirred solution of 2-bromo-4-((tert-butyldimethylsilyloxy)methyl)thiazole (2.5 g, 8.11 mmol) in THF (30 mL) in a 100-mL 3-necked round-bottom flask under nitrogen was added n-BuLi in hexane (2.5 M, 4.86 mL, 12.2 mmol) dropwise at −78° C. in a liquid nitrogen/ethanol bath. The solution was stirred for 30 min at −78° C. To the above solution was added acetone (0.9 g, 16.2 mmol) dropwise at −78° C. Then the solution was stirred for 1 h at RT. The reaction was then quenched by the addition of 100 mL of water. The resulting solution was extracted with 3×100 ml of EtOAc, the organic layers were combined, dried over anhydrous Na₂SO₄, concentrated under vacuum. The residue was eluted from silica gel with EtOAc/PE (1:10). This resulted in 2.0 g (86%) of the title compound as yellow oil. MS-ESI: 288 (M+1).

### Step 4: 4-((Tert-butyldimethylsilyloxy)methyl)-2-(2-hydroxypropan-2-yl)thiazole-5-sulfonyl chloride

To a stirred solution of 2-(4-((tert-butyldimethylsilyloxy)methyl)thiazol-2-yl)propan-2-ol (2.0 g, 6.96 mmol) in THF (20 mL) in a 250-mL 3-necked round-bottom flask under nitrogen was added n-BuLi in hexane (2.5 M, 8.4 mL, 20.9 mmol) dropwise at −78° C. Then the solution was stirred for 30 min at −78° C. Then SO₂ was introduced into the solution bubbled for 10 min below −30° C. The solution was stirred for 30 min at RT. The resulting solution was concentrated under vacuum. The crude solid was dissolved in DCM (30 mL), following by the addition of NCS (1.4 g, 10.4 mmol) in portions at 0° C. in an ice/water bath. The solution was stirred for 2 h at RT. The resulting mixture was concentrated under vacuum. This resulted in 2.5 g crude title compound as a yellow solid. This reaction was monitored by adding MeOH, gave corresponding methyl sulfonate sinal, MS-ESI: 382 (M+1).

### Step 5: 4-((Tert-butyldimethylsilyloxy)methyl)-2-(2-hydroxypropan-2-yl)thiazole-5-sulfonamide

To a stirred solution of 4-((tert-butyldimethylsilyloxy)methyl)-2-(2-hydroxypropan-2-yl)thiazole-5-sulfonyl chloride (2.5 g crude) in DCM (30 mL) in a 100-mL round-bottom flask was bubbled NH₃(g) for 20 min at 0° C. in an ice/water bath. The resulting solution was stirred for 1 h at RT. The resulting mixture was concentrated. The residue was eluted from silica gel with EtOAc/PE (1:5). This resulted in 1.2 g (47.0% over 2 steps) of the title compound as yellow oil. MS-ESI: 367 (M+1).

### Step 6: N-(tert-butyldimethylsilyl)-4-((tert-butyldimethylsilyloxy)methyl)-2-(2-hydroxypropan-2-yl)thiazole-5-sulfonamide

To a stirred solution of 4-((tert-butyldimethylsilyloxy)methyl)-2-(2-hydroxypropan-2-yl)thiazole-5- sulfonamide (1.2 g, 3.27 mmol) in THF (20 mL) in a 100-mL round-bottom flask under nitrogen was added NaH (60% wt. oil dispersion, 392 mg, 9.8 mmol) in portions at 0° C. in an ice/water bath. After stirring for 15 min at RT, the solution of TBSCl (1.5 g, 9.82 mmol) in THF (5 mL) was added to the reaction mixture dropwise at 0° C. The resulting solution was stirred for 2 h at RT. The reaction was quenched by the addition of 100 mL of water. The resulting solution was extracted with 3×100 mL of EtOAc, the organic layers were combined, dried over anhydrous Na₂SO₄ and concentrated under vacuum. This resulted in 1.3 g (83%) of the title compound as yellow oil. MS-ESI: 481 (M+1).

### Step 7: N′-(tert-butyldimethylsilyl)-4-((tert-butyldimethylsilyloxy)methyl)-2-(2-hydroxypropan-2-yl)thiazole-5-sulfonimidamide

To a stirred solution of PPh₃Cl₂ (1.4 g, 4.06 mmol) in CHCl₃ (10 mL) in a 100-mL 3- necked round-bottom flask under nitrogen was added DIEA (1.05 g, 8.11 mmol) dropwise at 0° C. in an ice/water bath. The solution was stirred at RT for 20 min. To this solution was added N-(tert-butyldimethylsilyl)-4-((tert-butyldimethylsilyloxy)methyl)-2-(2-hydroxypropan-2-yl)thiazole-5-sulfonamide (1.3 g, 2.7 mmol) in CHCl₃ (10 mL) dropwise at 0° C. The resulting solution was stirred for 0.5 h at RT. The reaction solution was bubbled NH₃ (g) for 20 min at 0° C. The solution was stirred for 1 h at RT. The resulting solution was concentrated under vacuum. The residue was eluted from silica gel with EtOAc/PE (1:5). This resulted in 600 mg (46%) of the title compound as a yellow solid. MS-ESI: 480 (M+1).

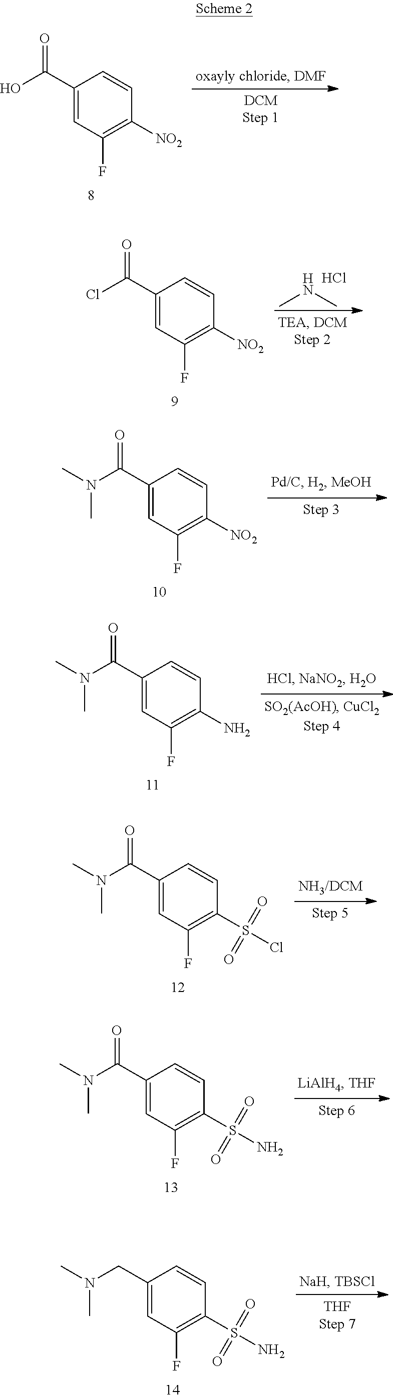

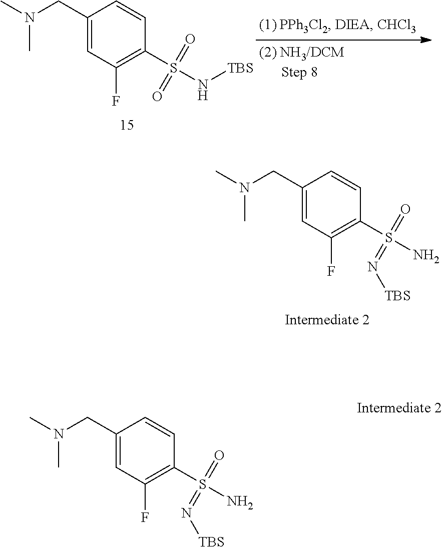

### N′-(tert-butyldimethylsilyl)-4-((dimethylamino)methyl)-2-fluorobenzenesulfonimidamide

### Step 1: 3-Fluoro-4-nitrobenzoyl chloride

To a stirred solution of 3-fluoro-4-nitrobenzoic acid (22.2 g, 120 mmol) in DCM (200 mL) in a 500-mL round-bottom flask under nitrogen was added DMF (0.2 mL), followed by the addition of oxalyl chloride (15 mL, 135 mmol) dropwise with stirring at 0° C. The resulting solution was stirred for 4 h at RT and then the solution was concentrated under vacuum. This resulted in 25 g crude title compound as yellow oil.

### Step 2: 3-Fluoro-N,N-dimethyl-4-nitrobenzamide

To a stirred solution of dimethylamine hydrochloride (9.8 g, 120 mmol) in DCM (200 mL) in a 500-mL round-bottom flask was added TEA (42 mL, 304 mmol) dropwise at RT. This was followed by the addition of 3-fluoro-4-nitrobenzoyl chloride (25 g, crude from last step) in DCM (100 mL) dropwise with stirring at 0° C. The resulting solution was stirred for 6 h at RT and then was concentrated under vacuum. The crude product was washed with 2×50 mL of water. The solids were collected by filtration. This resulted in 17.9 g (70% over 2 steps) of the title compound as a white solid. MS-ESI: 213 (M+1).

### Step 3: 4-Amino-3-fluoro-N,N-dimethylbenzamide

To a stirred solution of 3-fluoro-N,N-dimethyl-4-nitrobenzamide (17.6 g, 82.4 mmol) in MeOH (100 mL) in a 250-mL round-bottom flask under nitrogen was added Pd/C (10% wt., 1.0 g) at 0° C. in an ice/water bath. The flask was evacuated and flushed three times with hydrogen. The resulting solution was stirred for 12 h at RT under hydrogen atmosphere with a balloon. The Pd/C catalysts were filtered out and the filter cake was washed with MeOH (3×20 mL), the filtrate and wash were concentrated under vacuum. This resulted in 13 g (96%) of the title compound as a off-white solid. MS-ESI: 183 (M+1).

### Step 4: 4-(dimethylcarbamoyl)-2-fluorobenzenesulfonyl chloride

To a stirred solution of 4-amino-3-fluoro-N,N-dimethylbenzamide (3.35 g, 18.3 mmol) in HCl (6 M, 12 mL) in a 50-mL round-bottom flask was added NaNO₂ (1.5 g, 21.7 mmol) in water (3 mL) dropwise with stirring at 0° C. over 10 min. The resulting solution was stirred for 30 min at 0° C., this solution was assigned as solution A. Then to a stirred solution of CuCl₂ (4.8 g, 35.7 mmol) in AcOH (100 mL) in a 500-mL single necked round-bottom flask was bubbled SO₂ (g) at RT for 20 min, this solution was assigned as solution B. To the solution B was added solution A dropwise with stirring at 0° C. The resulting solution was stirred for 2 h at RT. The residue was diluted with 200 mL of water and extracted with 3×200 mL of DCM. The organic layers were combined and dried over anhydrous Na₂SO₄ and concentrated under vacuum. This resulted in 5.0 g (crude) of the title compound as yellow oil. The crude product was used in the next step.

### Step 5: 3-Fluoro-N,N-dimethyl-4-sulfamoylbenzamide

To a stirred solution of 4-(dimethylcarbamoyl)-2-fluorobenzenesulfonyl chloride (5.0 g, crude from last step) in DCM (20 mL) in a 250-mL round-bottom flask was bubbled NH3(g) for 20 min at 0° C. The resulting solution was stirred for 2 h at RT and then was concentrated under vacuum. The resulting mixture was washed with 3×100 mL of EtOAc. The solids were filtered out. The resulting filtrate was concentrated under vacuum. This resulted in 3.1 g (68.2% over two steps) of the title compound as a white solid. MS-ESI: 245 (M−1).

Step 6: 4-((Dimethylamino)methyl)-2-fluorobenzenesulfonamide

To a stirred solution of 3-fluoro-N,N-dimethyl-4-sulfamoylbenzamide (3.1 g, 12.6 mmol) in THF (80 mL) in a 250-mL 3-necked round-bottom flask under nitrogen was added LiAlH₄ (958 mg, 25.2 mmol) in portions at 0° C. The resulting solution was stirred for 12 h at RT and then was quenched by the addition of 10 mL of water/ice. The insoluble matter was filtered out and the filter cake was washed with MeOH (3×100 mL). The filtrate and wash were combined and concentrated under vacuum. The residue was eluted from silica gel with a gradient of EtOAc/PE (6:1 to 8:1). This resulted in 7.0 g (38%) of the title compound as a white solid. MS-ESI: 233 (M+1).

Steps 7-8 used similar procedures for converting compound 6 to Intermediate 1 shown in Scheme 1 to afford Intermediate 2 from compound 14. MS-ESI: 346 (M+1).

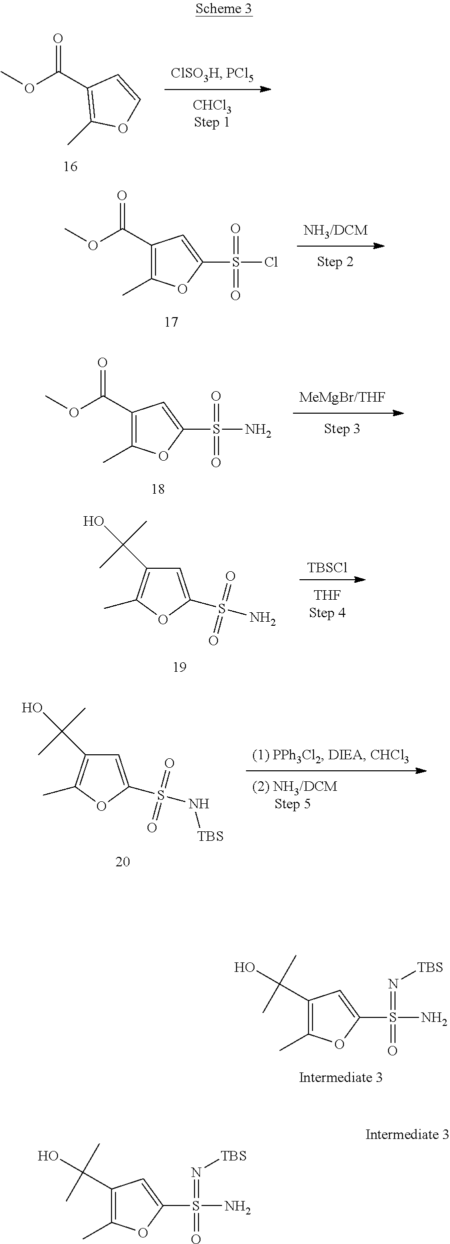

### N′-(tert-butyldimethylsilyl)-4-(2-hydroxypropan-2-yl)-5-methylfuran-2-sulfonimidamide

### Step 1: Methyl 5-(chlorosulfonyl)-2-methylfuran-3-carboxylate

To a stirred solution of methyl 2-methylfuran-3-carboxylate (7.0 g, 50 mmol) in CHCl₃ (200 mL) in a 500-mL 3-necked round-bottom flask was added chlorosulfonic acid (11.6 g, 100 mmol) dropwise at −10° C. Then the reaction mixture was stirred for 48 h at RT. Then to the above mixture was added phosphorus pentachloride (22.9 g, 110 mmol) in small portions at -10° C. The resulting solution was stirred for 0.5 h at 50° C. The reaction was quenched by pouring into 200 mL of water/ice slowly. The resulting mixture was extracted with 3×200 mL of DCM. The organic layers were combined and dried over anhydrous Na₂SO₄, then concentrated under vacuum. This resulted in 7.5 g (crude, 63%) of the title compound as light brown oil. The crude product was used in the next step.

### Step 2: Methyl 2-methyl-5-sulfamoylfuran-3-carboxylate

To a stirred solution of methyl 5-(chlorosulfonyl)-2-methylfuran-3-carboxylate (7.5 g, crude) in DCM (75 mL) in a 250-mL round-bottom flask was bubbled NH3(g) for 20 min at 0° C. in an ice/water bath. The resulting solution was stirred for 3 h at RT and concentrated under vacuum. The residue was eluted from silica gel with a gradient of EtOAc/PE (1:4 to 1:2). This resulted in 5.0 g (46% over two steps) of the title compound as a light yellow solid. MS-ESI: 218 (M−1).

### Step 3: 4-(2-Hydroxypropan-2-yl)-5-methylfuran-2-sulfonamide

To a stirred solution of methyl 2-methyl-5-sulfamoylfuran-3-carboxylate (3.7 g, 16.9 mmol) in THF (100 mL) in a 250-mL 3-necked round-bottom flask was added MeMgBr in THF (3 M, 25 mL) dropwise at −10° C. The resulting solution was stirred for 10 h at RT. Then the reaction solution was quenched with 50 mL of NH₄Cl (sat.). The resulting solution was extracted with 3×100 mL of EtOAc. The organic layers were combined and dried over anhydrous Na₂SO₄ and concentrated under vacuum. The residue was eluted from silica gel with a gradient of EtOAc/PE (1:3 to 1:1). This resulted in 2.6 g (75%) of the title compound as a light yellow solid. MS-ESI: 218 (M−1).

### Step 4: N-(tert-butyldimethylsilyl)-4-(2-hydroxypropan-2-yl)-5-methylfuran-2-sulfonamide

To a stirred solution of 4-(2-hydroxypropan-2-yl)-5-methylfuran-2-sulfonamide (1.0 g, 4.56 mmol) in THF (100 mL) in a 250-mL round-bottom flask under nitrogen was added NaH (60% wt. dispersion in mineral oil, 365 mg, 9.12 mmol) in portions at 0° C. in an ice/water bath. The solution was stirred for 10 min at RT. To the stirred solution was added TBSCl (3.4 g, 22.6 mmol) in portions at 0° C. The resulting solution was stirred for 14 h at RT and then was diluted with 100 mL of water. The resulting mixture was extracted with 3×50 mL of DCM and the organic layers were combined and concentrated under vacuum. The residue was eluted from silica gel with a gradient of EtOAc/PE (1:10 to 1:3). This resulted in 1.4 g (92%) of the title compound as a white solid. MS-ESI: 332 (M−1).

### Step 5: N′-(tert-butyldimethylsilyl)-4-(2-hydroxypropan-2-yl)-5-methylfuran-2-sulfonimidamide

To a stirred solution of PPh₃Cl₂ (3.0 g, 10.2 mmol) in CHCl₃ (100 mL) in a 250-mL 3-necked round-bottom flask was added DIEA (2.63 g, 20.4 mmol) dropwise at 0° C. After stirred at 0° C. for 10 min, to the above mixture was added a solution of N-(tert-butyldimethylsilyl)-4-(2-hydroxypropan-2-yl)-5-methylfuran-2-sulfonamide (2.3 g, 6.8 mmol) in CHCl₃ (10 mL) dropwise with stirring at 0° C. The resulting solution was allowed to react for 30 min at RT. To the mixture was bubbled NH₃(g) for 20 min at 0° C. The resulting solution was stirred for 2 h at RT. The reaction was then quenched by the addition of 100 mL of water. The resulting solution was extracted with 3×50 mL of DCM and the organic layers were combined and concentrated under vacuum. The residue was eluted from silica gel with a gradient of EtOAc/PE (1:10 to 1:3). This resulted in 0.80 g (53%) of the title compound as a light yellow solid. MS-ESI: 333 (M+1).

TABLE 1
The Intermediates in the following Table were prepared using the similar procedures for
converting compound 16 to Intermediate 3 shown in Scheme 3 from appropriated starting
materials.

| Intermediate # | Structure | IUPAC Name | Exact Mass [M + H]⁺ |
|---|---|---|---|
| Intermediate 4 | 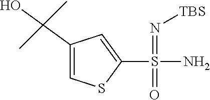 | N′-(tert-butyldimethylsilyl)-4-(2- hydroxypropan-2-yl)thiophene-2- sulfonimidamide | 335 |

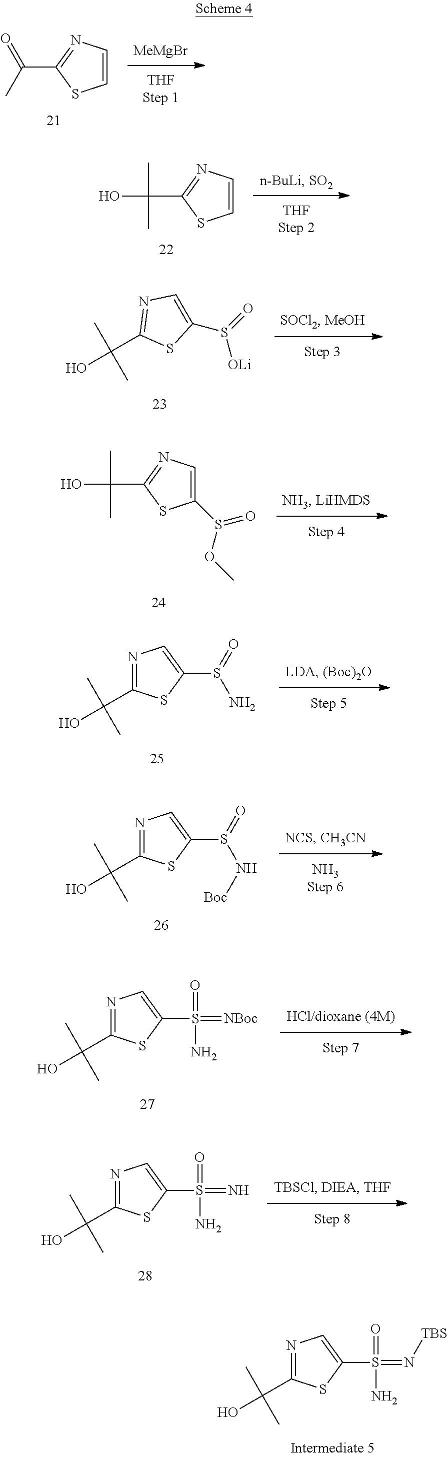

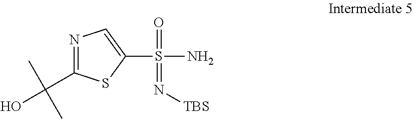

### N′-(tert-butyldimethylsilyl)-2-(2-hydroxypropan-2-yl)thiazole-5-sulfonimidamide

### Step 1: 2-(Thiazol-2-yl)propan-2-ol

To a stirred solution of 1-(thiazol-2-yl)ethanone (200 g, 1.6 mol) in THF (4 L) in a 10-L 4-necked round-bottom flask under nitrogen was added MeMgBr in THF (3 M, 942 mL, 2.83 mol) dropwise with stirring at 0° C. The mixture was stirred at 0° C. for 2 h. After warmed the mixture to RT, the solution was stirred for an additional 16 h. Then the reaction was quenched by the addition of 3 L of NH₄Cl (sat.). The resulting solution was extracted with 3×1 L of EtOAc. The organic layers were combined and dried over anhydrous Na₂SO₄, then concentrated under vacuum. The residue was eluted from silica gel with a gradient of EtOAc/PE (1:3 to 1:1). This resulted in 210 g (93%) of the title compound as brown oil. MS-ESI: 144 (M+1). ¹H NMR (400 MHz, DMSO-d6) δ 7.68 (d, J=3.2 Hz, 1H), 7.54 (d, J=3.2 Hz, 1H), 5.94 (s, 1H), 1.51 (s, 6H).

### Step 2: Lithium 2-(2-hydroxypropan-2-yl)thiazole-5-sulfinate

To a stirred solution of 2-(thiazol-2-yl)propan-2-ol (50 g, 349 mmol) in THF (1.5 L) in a 10-L 4-necked round-bottom flask under nitrogen was added n-BuLi in hexane (2.5 M, 350 mL) dropwise with stirring at −78° C. The mixture was stirred at −78° C. for 1 h. Then SO₂ was bubbled into the mixture for 15 min below −30° C. The mixture was stirred for an additional 1 h at RT and then was concentrated under vacuum. This resulted in 87 g (crude) of the title compound as a light yellow solid. The crude product was used directly in the next step. MS-ESI: 206 (M−1)

### Step 3: Methyl 2-(2-hydroxypropan-2-yl)thiazole-5-sulfinate

To a stirred solution of lithium 2-(2-hydroxypropan-2-yl)thiazole-5-sulfinate (87 g, crude) in MeOH (500 mL) in a 2-L 3-necked round-bottom flask was added SOCl₂ (43 g, 360 mmol) dropwise with stirring at 0° C. The mixture was stirred overnight at RT, then was concentrated under vacuum. The residue was diluted with 500 mL of EtOAc. The resulting solution was washed with 2×200 mL of water and 2×200 mL of brine. The solution was dried over anhydrous Na₂SO₄, concentrated under vacuum. This resulted in 72 g (crude) title compound as light yellow oil. The crude product was used directly in the next step. MS-ESI: 222[M+1]. ¹H NMR (400 MHz, DMSO-d₆) δ 8.15 (s, 1H), 6.32 (s, 1H), 3.65 (s, 3H), 1.54 (s, 3H), 1.53 (s, 3H).

### Step 4: 2-(2-Hydroxypropan-2-yl)thiazole-5-sulfinamide

To a stirred solution of methyl 2-(2-hydroxypropan-2-yl)thiazole-5-sulfinate (72 g, 326 mmol) in THF (500 mL) in a 10-L 4-necked round-bottom flask was added NH₃ in THF (0.5 M, 2.0 L). After cooling to −78° C., LiHMDS in THF (1 M, 2.0 L) was added to the mixture under nitrogen dropwise at 0° C. with stirring. Then the mixture was stirred at −78° C. for 2 h. The reaction was quenched by the addition of 500 mL of NH₄Cl (sat.). The resulting solution was extracted with 3×300 mL of EtOAc. The organic layers were combined, dried over anhydrous Na₂SO₄, then concentrated under vacuum. This resulted in 32 g (crude) title compound as brown oil. The crude product was used directly in the next step. MS-ESI: 207 [M+1].¹H NMR (400 MHz, DMSO-d₆) δ 7.77 (s, 1H), 6.73 (s, 2H), 6.17 (s, 1H), 1.53 (s, 3H), 1.52 (s, 3H).

### Step 5: Tert-butyl 2-(2-hydroxypropan-2-yl)thiazol-5-ylsulfinylcarbamate

To a stirred solution of 2-(2-hydroxypropan-2-yl)thiazole-5-sulfinamide (32 g, crude) in THF (300 mL) in 1-L 3-necked round-bottom flask under nitrogen was added LDA in THF (2 M, 116 mL) dropwise with string at 0° C. The mixture was stirred at 0° C. for 1 h, then (Boc)₂O (33.8 g, 155 mmol) was added in portions at 0° C. The mixture was warmed to RT and stirred for an additional 2 h. The reaction was quenched with 200 mL of ice/water (200 mL), the pH value of the solution was adjusted to 6 with FA. The resulting solution was extracted with 3×200 mL of EtOAc. The organic layers were combined, dried over anhydrous Na₂SO₄, then concentrated under vacuum. The residue was eluted from silica gel with a gradient of EtOAc/PE (1:2 to 1:1). This resulted in 19 g (18% over 4 steps) title compound as a white solid. MS-ESI: 307 [M+1].

### Step 6: N-(tert-butyldimethylsilyl)-2-(2-hydroxypropan-2-yl)thiazole-5-sulfonimidamide

To a stirred solution of tent-butyl 2-(2-hydroxypropan-2-yl)thiazol-5-ylsulfinylcarbamate (19 g, 62 mmol) in MeCN (200 mL) in a 1-L 3-necked round-bottom under nitrogen was added NCS (9.8 g, 74 mmol) in small portions at 0° C. The mixture was stirred for 1 h at RT. Then NH₃ was bubbled into the mixture for 15 min at 0° C. The mixture was stirred at RT for 2 h and then was concentrated under vacuum. The residue was eluted from gel with a gradient of EtOAc/PE (1:2 to 1:1). This resulted in 13 g (65%) of the title compound as a white solid. MS-ESI: 322 [M+1]. ¹H NMR (300 MHz, DMSO-d₆) δ 7.99 (s, 1H), 7.72 (s, 2H), 6.29 (s, 1H), 1.49 (s, 3H), 1.48 (s, 3H), 1.27 (s, 9H).

### Step 7: Synthesis of 2-(2-hydroxypropan-2-yl)thiazole-5-sulfonimidamide

The solution of tert-butyl 2-(2-hydroxypropan-2-yl)thiazole-5-sulfonimidoylcarbamate (3.21 g, 10 mmol) in HCl/dioxane (4 M, 50 mL) in a 250-mL round-bottom flask was stirred for 1 h at RT. The solution was concentrated to give the title compound (3.2 g, crude, yellow oil). MS-ESI: 222 [M+1]

### Step 8: N′-(tert-butyldimethylsilyl)-2-(2-hydroxypropan-2-yl)thiazole-5-sulfonimidamide

To a stirred solution of 2-(2-hydroxypropan-2-yl)thiazole-5-sulfonimidamide (3.2 g, crude from last step) in THF (100 mL) in a 250-mL round-bottom flask was added DIEA (3.87 g, 30 mmol) at RT, followed by the addition of TBSCl (3.0 g, 20 mmol) in THF (10 mL) dropwise at RT. The resulting solution was stirred for 6 h at RT. The solution was concentrated, the crude product was eluted from silica gel with EtOAc/PE (1:1) to give the tilte compound (2.3 g, 68.5% over two steps, yellow solid). MS-ESI: 336 [M+1]

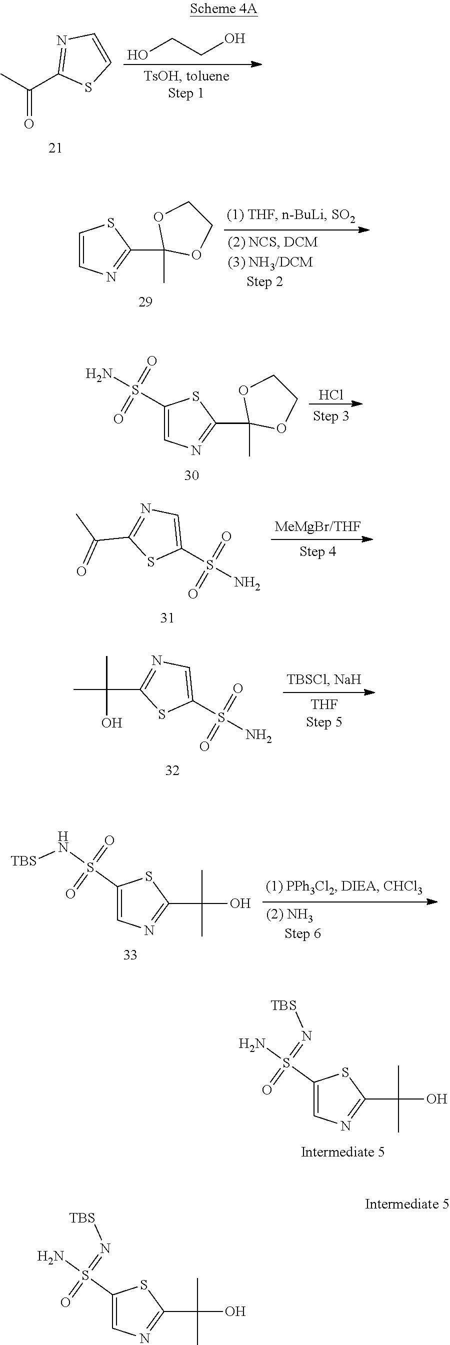

### N′-(tert-butyldimethylsilyl)-2-(2-hydroxypropan-2-yl)thiazole-5-sulfonimidamide

### Step 1: 2-(2-Methyl-1,3-dioxolan-2-yl)thiazole

To a stirred solution of 1-(thiazol-2-yl)ethanone (20 g, 157.0 mmol) in toluene (300 mL) and ethane-1,2-diol (19.5 g, 314 mmol) in a 500-mL round-bottom flask was added TsOH (2.7 g, 15.7 mmol) in portions. The resulting solution was refluxed overnight and water was separated from the solution during the reflux. The resulting solution was diluted with 200 mL of water and extracted with 2×100 mL of EtOAc. The organic layers were combined, dried over anhydrous Na₂SO₄, and then concentrated under vacuum. This resulted in 26.6 g (99%) of the title compound as light yellow oil. MS-ESI: 172.0 (M+1).

### Step 2: 2-(2-Methyl-1,3-dioxolan-2-yl)thiazole-5-sulfonamide

To a stirred solution of 2-(2-methyl-1,3-dioxolan-2-yl)thiazole (14 g, 81.6 mmol) in THF (200 mL) in a 500-mL 3-necked round-bottom flask under nitrogen was added n-BuLi in hexane (2.5 M, 35.2 mL, 88 mmol) dropwise at −78° C. The resulting solution was stirred for 0.5 h at −78° C. and then SO₂ was introduced into the above reaction mixture bubbled for 15 min at 0° C. The reaction was slowly warmed to RT. The reaction was stirred for 2 h at RT. Then the reaction solution was concentrated under vacuum. The mixture was dissolved in DCM (200 mL). Then NCS (12.8 g, 96 mmol) was added to the mixture solution in portions at 0° C. The resulting solution was stirred for 1 h at RT. Then the solution was washed with 3×200 mL H₂O. The organic layer was dried over anhydrous Na₂SO₄. The resulting solution was bubbled NH3(g) for 20 min at 0° C. The resulting mixture was stirred for 3 h at RT and concentrated under vacuum. The residue was eluted from silica gel with a gradient of EtOAc/PE (1:20 to 1:5). This resulted in 12.5 g (61%) of the title compound as a yellow solid. MS-ESI: 251.0 (M+1).

### Step 3: 2-Acetylthiazole-5-sulfonamide

To a stirred solution of 2-(2-methyl-1,3-dioxolan-2-yl)thiazole-5-sulfonamide (12.5 g, 50.0 mmol) in THF (125 mL) in a 250-mL round-bottom flask was added HCl (4 N, 50.0 mL) dropwise at RT. The resulting solution was stirred for 6 h at 70° C. The resulting solution was diluted with 100 mL of water and extracted with 2×200 mL of EtOAc. The organic layers were combined, dried over anhydrous Na₂SO₄ and concentrated under vacuum. The residue was eluted from silica gel with a gradient of EtOAc/PE(1:2 to 1:1). This resulted in 9.3 g (90%) of the title compound as a yellow solid. MS-ESI: 207.0 (M+1).

Steps 4-6 used the same procedures for converting compound 18 to Intermediate 3 shown in Scheme 3 to afford Intermediate 5 from compound 31. MS-ESI: 336 (M+1).

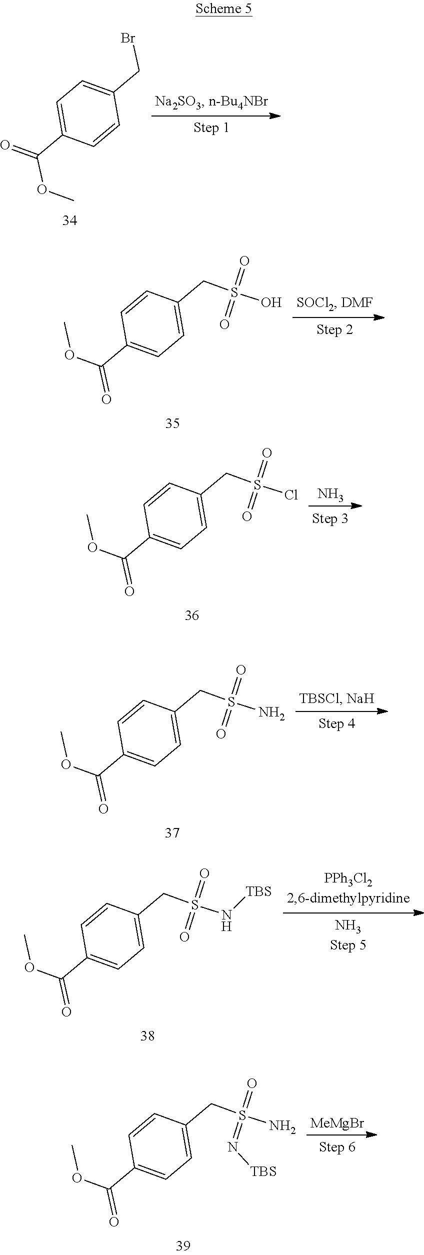

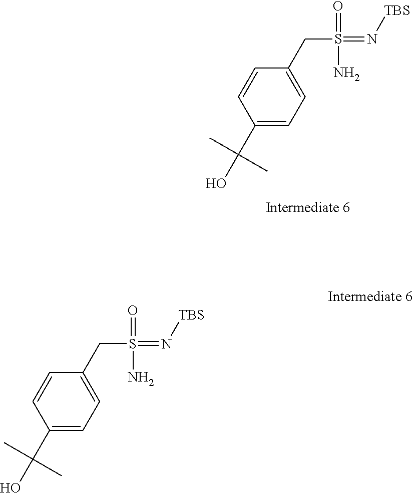

### N′-(tert-butyldimethylsilyl)-1-(4-(2-hydroxypropan-2-yl)phenyl)methanesulfonimidamide

### Step 1: (4-(Methoxycarbonyl)phenyl)methanesulfonic acid

To a stirred solution of methyl 4-(bromomethyl)benzoate (10 g, 44 mmol) in water (30 mL) in a 250-mL round-bottom flask was added Na₂SO₃ (7.18 g, 57 mmol) and tetrabutylammonium bromide (708 mg, 2.2 mmol) at RT. The resulting solution was stirred for 15 h at 80° C. The resulting mixture was concentrated under reduced pressure. The residue was washed with 50 mL of isopropyl alcohol. This resulted in 15 g (crude) of the title compound as a white solid. MS-ESI: 229 (M−1).

### Step 2: Methyl 4-((chlorosulfonyl)methyl)benzoate

To a stirred solution of (4-(methoxycarbonyl)phenyl)methanesulfonic acid (15.0 g, crude from last step) in SOCl₂ (100 mL) in a 250-mL round-bottom flask under nitrogen was added DMF (2 mL) dropwise at 0° C. The resulting solution was stirred for 30 min at 0° C. then allowed to react, with stirring, for an additional 5 h at RT. The reaction mixture was concentrated under reduced pressure and quenched by the addition of 100 mL of water/ice. The resulting solution was extracted with 3×200 mL of DCM. The organic layers were combined, dried over anhydrous Na₂SO₄. This resulted in the title compound as a yellow solution which was used into next step directly. MS-ESI: 249/251 (M+1).

### Step 3: Methyl 4-(sulfamoylmethyl)benzoate

To a stirred solution of methyl 4-((chlorosulfonyl)methyl)benzoate in DCM (600 mL, crude from last step) was bubbled NH₃(g) for 15 min at 0° C. The resulting solution was stirred for 30 min at 0° C. then allowed to react, with stirring, for an additional 12 h at RT. The residue was eluted from silica gel with EtOAc/hexane (2:3). This resulted in 4.5 g (45.0% over 3 steps) of the title compound as a yellow solid. MS-ESI: 230 (M+1).

### Step 4: Methyl 4-((N-(tert-butyldimethylsilyl)sulfamoyl)methyl)benzoate

To a stirred solution of methyl 4-(sulfamoylmethyl)benzoate (1.40 g, 6.12 mmol) in THF (20 mL) in a 100-mL round-bottom flask under nitrogen was added NaH (60% wt. oil dispersion, 468 mg, 12.2 mmol) in portions at 0° C. The resulting solution was stirred for 30 min at 0° C. Then TBSCl (1.38 g, 9.16 mmol) was added to the reaction mixture in portions at 0° C. The resulting solution was stirred for 5 h at RT. The reaction was then quenched by the addition of 100 mL of water. The resulting solution was extracted with 3×100 mL of EtOAc. The organic layers were combined, dried over anhydrous Na₂SO₄, concentrated under reduced pressure. This resulted in 2 g (95.0%) of the title compound as a yellow solid. MS-ESI: 344 (M+1).

### Step 5: Methyl 4-((N′-(tert-butyldimethylsilyl)sulfamidimidoyl)methyl)benzoate

To a stirred solution of PPh₃Cl₂ (7.76 g, 23.3 mmol) in CHCl₃ (20 mL) a 250-mL 3-necked round-bottom flask under nitrogen was added 2,6-lutidine (4.99 g, 46.6 mmol) in CHCl₃ (5 mL) dropwise at 0° C. The resulting solution was stirred for 15 min at 0° C. This was followed by the addition of methyl 4-[[(tert-butyldimethylsilyl)sulfamoyl]methyl]benzoate (2.0 g, 5.82 mmol) in CHCl₃ (10 mL) dropwise at 0° C. The resulting solution was stirred for 30 min at RT. To the above mixture was bubbled NH₃(g) for 10 min at 0° C. The resulting solution was allowed to react, with stirring, for an additional 12 h at RT. The reaction mixture was concentrated under reduced pressure. The residue was eluted from silica gel with EtOAc/hexane (2:3). This resulted in 1.67 g (83.5%) of the title compound as a yellow solid. MS-ESI: 343 (M+1).

### Step 6: N′-(tert-butyldimethylsilyl)-1-(4-(2-hydroxypropan-2-yl)phenyl)methanesulfonimidamide

To a stirred solution of methyl 4-((N′-(tert-butyldimethylsilyl)sulfamidimidoyl)methyl)benzoate (1.4 g, 4.09 mmol) in THF (25 mL) under nitrogen was MeMgBr in THF (3 M, 5.5 mL, 16.5 mmol) dropwise at 0° C. The resulting solution was stirred for 16 h at RT. The reaction was quenched with water/ice (100 mL). The resulting solution was extracted with 3×100 mL of EtOAc. The organic layers were combined, dried over anhydrous Na₂SO₄, concentrated under reduced pressure. The residue was eluted from silica gel with EtOAc/hexane (1:1). This resulted in 898 mg (64.1%) of the title compound as a yellow solid. MS-ESI: 343 (M+1).

Schemes for phenylacetic acids Intermediates: Schemes 6-10 illustrate the preparation of RHS intermediates.

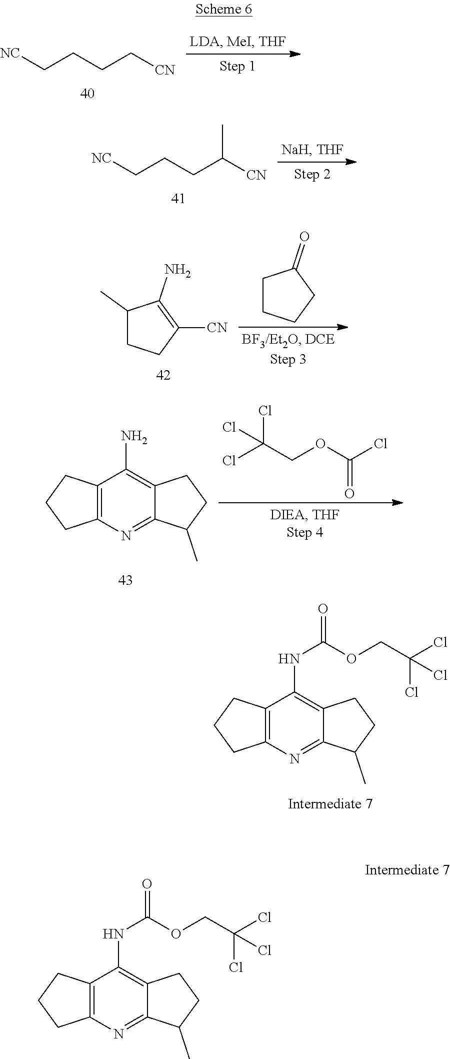

### 2,2,2-Trichloroethyl (3-methyl-1,2,3,5,6,7-hexahydrodicyclopenta[b,e]pyridin-8-yl)carbamate

### Step 1: 2-Methylhexanedinitrile

To a stirred of solution of LDA in THF (2 M, 76.4 mL, 153 mmol) in a 1-L 3-necked round-bottom flask under nitrogen was added a cold solution of adiponitrile (15 g, 139 mmol) in THF (100 mL) dropwise at −78° C. in a liquid nitrogen/EtOH bath. The resulting solution was stirred for 1 h at −78° C. To the reaction was added CH₃I (21.7 g, 153 mmol) in THF (50 mL) dropwise at −78° C. Then the reaction was stirred for 1 h at 0° C. The reaction was quenched with sat. NH₄Cl (100 mL). The THF was removed under reduced pressure, the mixture was extracted with 3×200 mL EtOAc and the combined organic layers were dried over anhydrous Na₂SO₄ and concentrated under vacuum. This resulted in 15 g (crude) title compound as yellow oil. MS-ESI: 123 (M+1).

### Step 2: 2-Amino-3-methylcyclopent-1-ene-1-carbonitrile

To a stirred solution of 2-methylhexanedinitrile (15 g, 123 mmol) in THF (450 mL) in a 1-L 3-necked round bottom flask under nitrogen was added NaH (60% wt. dispersion in mineral oil, 9.84 g, 246 mmol) in portions at 0° C. in an ice/EtOH bath. The reaction was stirred for 30 min at 0° C. and then overnight at 80° C. The reaction was quenched with 200 mL of H₂O. The mixture was extracted with 3×300 mL of EtOAc. The combined organic layers were dried over anhydrous Na₂SO₄ and concentrated under reduced pressured. The crude product was eluted from silica gel with EtOAc/PE (1:3). This resulted in 4.8 g (28% over two steps) of the title compound as a yellow solid. MS-ESI: 123 (M+1).

### Step 3: 3-Methyl-1,2,3,5,6,7-hexahydrodicyclopenta[b,e]pyridin-8-amine

To a stirred solution of 2-amino-3-methylcyclopent-1-ene-1-carbonitrile (6.1 g, 50 mmol) in DCE (125 mL) in a 250-mL 3-necked round-bottom flask under nitrogen was added cyclopentanone (8.4 g, 100 mmol) at RT. Then BF₃.Et₂O (46.5% wt., 14.5 g) was added to this solution at 0° C. in an ice bath. The reaction was heat to 75° C. for 6 h. The reaction cooled to RT, then quenched by the addition of 100 mL of water/ice, extracted with DCM (2×50 mL). The aqueous phase was collected and adjusted the pH value to 14 with NaOH (6 M) until the solid was precipitated. The solids were collected by filtration. The filter cake was washed with water (150 mL), then dried by infra-red drying, this resulted of the title compound (7.0 g, yield 80%, light yellow solid). MS-ESI: 175 (M+1).

### Step 4: 2,2,2-Trichloroethyl (3-methyl-1,2,3,5,6,7-hexahydrodicyclopenta[b,e]pyridin-8-yl)carbamate

To a stirred solution of 3-methyl-1,2,3,5,6,7-hexahydrodicyclopenta[b,e]pyridin-8-amine (427 mg, 2.27 mmol) in THF (30 mL) in a 100-mL round-bottom flask under nitrogen was added DIEA (587 mg, 4.54 mmol) at RT, followed by the addition of 2,2,2-trichloroethyl carbonochloridate (962 mg, 4.54 mmol) dropwise at RT. The resulting solution was stirred overnight at RT. The reaction solution was quenched with H₂O (10 mL). Th mixture was extracted with 3×50 mL EtOAc. The organic layers were combined and dried over anhydrous Na₂SO₄ and concentrated under vacuum. The residue was eluted from silica gel with EtOAc/PE(1:1). This resulted in 610 mg (74%) of the title compound as a brown yellow solid. MS-ESI: 363/365/367 (M+1).

TABLE 2
The Intermediates in the following Table were prepared using the similar procedures for
converting compound 40 to Intermediate 7 shown in Scheme 6 from appropriated starting
materials.

| Intermediate # | Structure | IUPAC Name | Exact Mass [M + H]⁺ |
|---|---|---|---|
| Intermediate 8 | 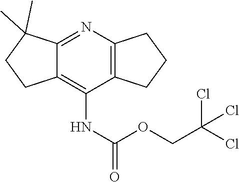 [SMILES: CC1(C)CCc2c1nc1c(c2NC(=O)OCC(Cl)(Cl)Cl)CCC1] | 2,2,2-Trichloroethyl (3,3-dimethyl- 1,2,3,5,6,7- hexahydrodicyclopenta[b,e]pyridin- 8-yl)carbamate | 377/379/381 |

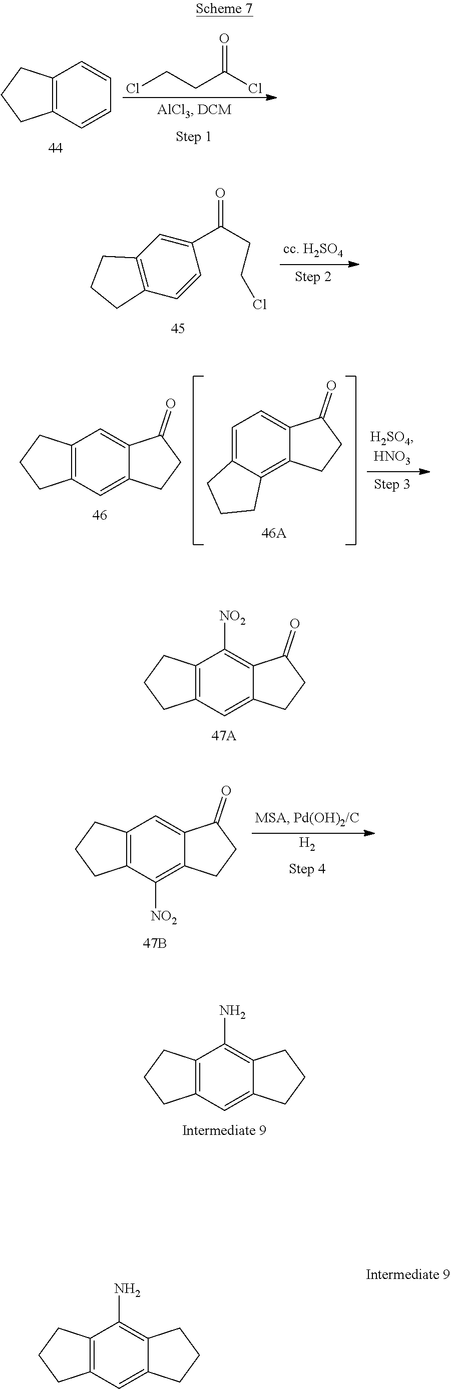

### 1,2,3,5,6,7-Hexahydro-s-indacen-4-amine

### Step 1: 3-Chloro-1-(2,3-dihydro-1H-inden-5-yl)propan-1-one

To a stirred solution of AlCl₃ (111 g, 834 mmol) in DCM (1200 mL) in a 3000-mL round-bottom flask under nitrogen was added a mixture of 2,3-dihydro-1H-indene (90 g, 762 mmol) and 3-chloropropanoyl chloride (96.3 g, 759 mmol) in DCM (300 mL) dropwise with stirring at −10° C. in 30 min. The resulting solution was stirred for 16 h at RT. Then the reaction mixture was added dropwise to cold HCl (3 N, 1200 mL) over 45 min at −10° C. The resulting solution was extracted with 3×600 mL of DCM and the organic layers were combined, dried over anhydrous Na₂SO₄, then concentrated under vacuum. This resulted in 160.5 g (crude) of the title compound as a yellow solid. The crude product was used in the next step. MS-ESI: 209/211 (M+1).

### Step 2: 1,2,3,5,6,7-Hexahydro-s-indacen-1-one

The solution of 3-chloro-1-(2,3-dihydro-1H-inden-5-yl)propan-1-one (160.5 g, 759 mmol) in conc. H₂SO₄ (900 mL) in a 1000-mL round-bottom flask was stirred for 16 h at 55° C. The reaction was quenched by adding the reaction mixture carefully to 4500 mL of water/ice. The solids were collected by filtration and dried over infrared lamp for 24 h. The crude mixture was purified by chromatography and eluted with EtOAc/PE (1:100). This resulted in 10 g (7.6%) of 1,6,7,8-tetrahydro-as-indacen-3(2H)one (Cpd 46A) and 112.2 g (85%) of the title compound as a yellow solid. Compound 46: ¹H NMR (400 MHz, DMSO-d₆) δ 7.44 (s, 1H), 7.39 (s, 1H), 3.20-2.75 (m, 6H), 2.70-2.60 (m, 2H), 2.20-1.90 (m, 2H). Compound 46A: ¹H NMR (400 MHz, DMSO-d₆) δ 7.49 (d, J=8.0 Hz, 1H), 7.31 (d, J=8.0 Hz, 1H), 3.20-2.90 (m, 4H), 2.90-2.75 (m, 2H), 2.70-2.60 (m, 2H), 2.20-1.90 (m, 2H)

### Step 3: Mixture of 4-nitro-2,3,6,7-tetrahydros-indacen-1(5H)-one and 8-nitro-2,3,6,7-tetrahydros-indacen-1(5H)-one

To a stirred solution of 1,2,3,5,6,7-hexahydro-s-indacen-1-one (80 g, 464.5 mmol) in H₂SO₄ (500 mL) was added HNO₃ (58.5 g, 929 mmol) dropwise over 1 h at 0° C. The resulting solution was stirred for 1 h at RT. The reaction mixture was slowly added to a mixture of water/ice (1000 mL) and DCM (500 mL) with ice bath cooling. The organic layer was collected, dried over Na₂SO₄ and concentrated under vacuum. This resulted in 90 g (90%) of the mixture of 4-nitro-1,2,3,5,6,7-hexahydro-s-indacen-1-one and 8-nitro-2,3,6,7-tetrahydro-s-indacen-1(5H)-one as a yellow solid. MS-ESI: 218 (M+1).

### Step 4: 1,2,3,5,6,7-hexahydros-indacen-4-amine

To a stirred solution of the mixture of 4-nitro-1,2,3,5,6,7-hexahydro-s-indacen-1-one and 8-nitro-2,3,6,7-tetrahydro-s-indacen-1(5H)-one (21.7 g, 100 mmol) in MeOH (300 mL) was added MSA (11.5 g, 120 mmol) at RT. Then Pd(OH)₂/C (20% wt, 5.5 g) was added under nitrogen. The flask was evacuated and refilled three times with hydrogen. The resulting mixture was stirred for 16 h at RT under hydrogen with a balloon. The solids were filtered out and washed with MeOH (3×50 mL). The MeOH filtrate and wash was diluted with water (500 mL) and the pH was adjusted to 11 with 2N NaOH. The resulting slurry was filtered and the crude solids were recrystallized from MeOH/water (9:1) with heating. This resulted in 13.7 g (79%) of the title compound as an off-white solid. MS-ESI: 174 (M+1).

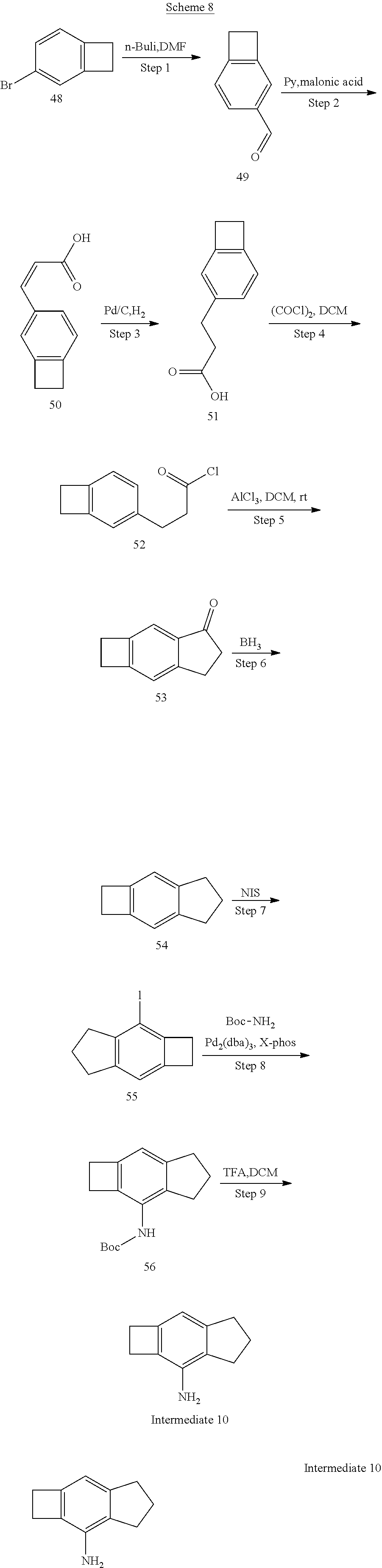

### 2,4,5,6-Tetrahydro-1H-cyclobuta[f]inden-3-amine

### Step 1: Bicyclo[4.2.0]octa-1(6),2,4-triene-3-carbaldehyde

To a stirred solution of 3-bromobicyclo[4.2.0]octa-1(6),2,4-triene (70 g, 382 mmol) in THF (300 mL) in a 500-mL round-bottom flask under nitrogen was added n-BuLi in hexane (2.5 M, 184 mL, 459 mmol) dropwise at −70° C. After addition, the reaction mixture was stirred at this temperature for 30 min. To this solution was added DMF (36.3 g, 497 mmol) dropwise with stirring at −70° C. The resulting solution was stirred for 30 min at −70° C. in a liquid nitrogen/EtOH bath. The reaction was slowly warmed to RT and then quenched by the addition of 100 mL of water. The resulting solution was extracted with 3×200 ml of DCM. The organic layers were combined and dried over anhydrous Na₂SO₄, and concentrated under reduced pressure. This resulted in 50 g (98.9%) of the title compound as light yellow oil. MS-ESI: 133 (M+1).

### Step 2: (Z)-3-(bicyclo[4.2.0]octa-1(6),2,4-trien-3-yl)acrylic acid

To a stirred solution of bicyclo[4.2.0]octa-1(6),2,4-triene-3-carbaldehyde (1.7 g, 12.9 mmol) in pyridine (20 mL) in a 250-mL round-bottom flask under nitrogen was added malonic acid (1.99 g, 19.2 mmol) and piperidine (110 mg, 1.29 mmol). The resulting solution was stirred overnight at 90° C. in an oil bath. The resulting mixture was concentrated under vacuum. This resulted in 2.1 g (93.7%) of the title compound as a light yellow solid. MS-ESI: 173 (M−1).

### Step 3: 3-(Bicyclo[4.2.0]octa-1(6),2,4-trien-3-yl)propanoic acid

To a stirred solution of (Z)-3-(1,2-dihydrocyclobutabenzen-4-yl)acrylic acid (2.1 g, 12.1 mmol) in MeOH (50 mL) in a 250-mL round-bottom flask was added Pd/C (10% wt., 200 mg) under nitrogen. The flask was evacuated and flushed three times with hydrogen. The resulting solution was stirred for 12 h at RT under hydrogen with a balloon. The Pd/C catalysts were filtered out, the filtrate was concentrated under vacuum. This resulted in 2.1 g (98.9%) of the title compound as a yellow solid. MS-ESI: 175 (M−1).

### Step 4: 3-(Bicyclo[4.2.0]octa-1(6),2,4-trien-3-yl)propanoyl chloride

To a stirred solution of 3-[bicyclo[4.2.0]octa-1(6),2,4-trien-3-yl]propanoic acid (10 g, 56.8 mmol) in DCM (100 mL) in a 250-mL round-bottom flask under nitrogen was added oxalyl chloride (7.2 g, 56.8 mmol) dropwise at 0° C. The resulting solution was stirred for 2 h at 0° C. in a water/ice bath. The resulting mixture was use in next step without further purification.

### Step 5: 1,2,5,6-Tetrahydro-4H-cyclobuta[f]inden-4-one

To a stirred solution of 3-[bicyclo[4.2.0]octa-1(6),2,4-trien-3-yl]propanoyl chloride in DCM (100 mL, from step 4) in a 500-mL round-bottom flask was added AlCl₃ (7.5 g, 56.8 mmol) in portions at 0° C. over 10 min. The resulting solution was stirred for 2 h at 0° C. in a water/ice bath. The reaction was then quenched by the addition of 200 mL of water. The resulting solution was extracted with 2×200 mL of DCM. The organic layers were combined, dried over anhydrous Na₂SO₄, and concentrated under vacuum. The residue was eluted from silica gel with EtOAc/PE (1:20 to 1:15). This resulted in 7.7 g (86.1%) of the title compound as a yellow solid. ¹H NMR (300 MHz, CDCl₃) δ 7.45 (s, 1H), 7.17 (s, 1H), 3.23- 3.21 (m, 4H), 3.18-3.00 (m, 2H), 2.73-2.63 (m, 2H).

### Step 6: 2,4,5,6-Tetrahydro-1H-cyclobuta[f]indene

To a stirred solution of 1,2,5,6-tetrahydrocyclobuta[f]inden-4-one (20 g, 126 mmol) in THF (200 mL) in a 500-mL round-bottom flask under nitrogen was added BH₃-Me₂S (10 M, 25.3 mL, 253 mmol) dropwise at 0° C. in an ice bath. The resulting solution was stirred for 14 h at 70° C. in an oil bath. The reaction was then quenched by the addition of 20 mL of MeOH. The resulting mixture was concentrated. The residue was eluted from silica gel with EtOAc/PE (1:100 to 1:50). This resulted in 15 g (82.3%) of the title compound as a light yellow solid. ¹H NMR (300 MHz, CDCl₃) δ 6.95 (s, 2H), 3.10 (s, 4H), 2.88 (t, J=7.4 Hz, 4H), 2.09-1.99 (m, 2H).

### Step 7: 3-Iodo-2,4,5,6-tetrahydro-1H-cyclobuta[f]indene

To a stirred solution of 2,4,5,6-tetrahydro-1H-cyclobuta[f]indene (15 g, 104 mmol) in DCE (200 mL) in a 500-mL round-bottom flask under nitrogen was added NIS (46.8 g, 208 mmol) in portions at RT. This was followed by the addition of AcOH (60 mL) and water (0.5 mL). The resulting solution was stirred for 14 h at 50° C. in an oil bath. The reaction was quenched with 30% Na₂SO₃ (aq.) (100 mL). The mixture was extracted with 3×100 mL of DCM. The organic layers were combined and dried over anhydrous Na₂SO₄, then concentrated. The residue was eluted from silica gel with PE. This resulted in 8.2 g (29.2%) of the title compound as yellow solid. MS-ESI: 271 (M+1).

### Step 8: tert-butyl 2,4,5,6-tetrahydro-1H-cyclobuta[f]inden-3-ylcarbamate

To a stirred solution of 3-iodo-2,4,5,6-tetrahydro-1H-cyclobuta[f]indene (5.0 g, 18.5 mmol) in toluene (100 mL) a 250-mL round-bottom flask under nitrogen was added tert-butyl carbamate (6.5 g, 55.5 mmol), X-phos (900 mg, 1.85 mmol), Pd₂(dba)₃ (800 mg, 0.93 mmol) and t-BuOK (6.2 g, 55.5 mmol). The resulting solution was stirred for 14 h at 100° C. in an oil bath. The resulting mixture was concentrated. The residue was eluted from a silica gel column with EtOAc/PE (1:50 to 1:20). This resulted in 3.0 g (83.3%) of the title compound as a white solid. MS-ESI: 260 (M+1). ¹H NMR (300 MHz, CDCl₃) δ 6.72 (s, 1H), 6.13 (br, 1H), 3.30-3.21 (m, 2H), 3.05-2.95 (m, 2H), 2.90 (t, J=7.4 Hz, 2H), 2.75 (t, J=7.4 Hz, 2H), 2.09-2.02 (m, 2H), 1.52 (s, 9H).

### Step 9: 2,4,5,6-Tetrahydro-1H-cyclobuta[f]inden-3-amine

To a stirred solution of tert-butyl 2,4,5,6-tetrahydro-1H-cyclobuta[f]inden-3- ylcarbamate (3.0 g, 11.6 mmol) in DCM (20 mL) was added TFA (5.0 mL) dropwise at 0° C. The resulting solution was stirred for 2 h at RT. The resulting solution was diluted with 50 mL of water. The pH value of the solution was adjusted to 10 with sat. aqueous Na₂CO₃. The resulting solution was extracted with 3×20 mL of DCM. The organic layers were combined and dried over anhydrous Na₂SO₄, then concentrated under reduced pressure. This resulted in 1.5 g (81.4%) of the title compound as a yellow solid. MS-ESI: 160 (M+1).

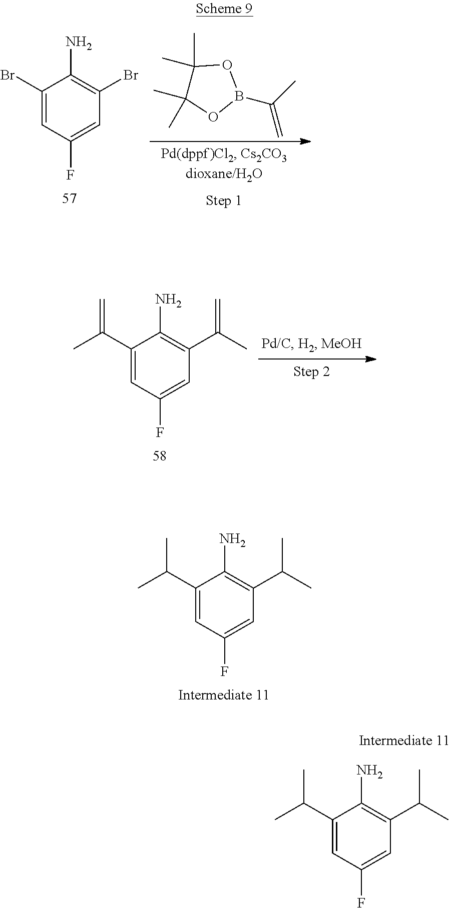

### 4-Fluoro-2,6-diisopropylbenzenamine

### Step 1: 4-Fluoro-2,6-bis(prop-1-en-2-yl)aniline

To a stirred solution of 2,6-dibromo-4-fluoroaniline (15 g, 55.8 mmol) in dioxane (150 mL), water(15 mL) in a 500-mL round-bottom flask under nitrogen was added Cs₂CO₃ (55 g, 169 mmol), 4,4,5,5-tetramethyl-2-(prop-1-en-2-yl)-1,3,2-dioxaborolane (25 g, 149 mmol) and Pd(dppf)Cl₂ (4 g, 5.47 mmol). The resulting solution was stirred for 15 h at 100° C. The reaction mixture was concentrated under vacuum. The residue was eluted from silica gel with a gradient of EtOAc/PE (1:10 to 1:8). This resulted in 9.2 g (86%) of the title compound as brown oil. MS-ESI: 192 (M+1).

### Step 2: 4-Fluoro-2,6-bis(propan-2-yl)aniline

To a stirred solution of 4-fluoro-2,6-bis(prop-1-en-2-yl)aniline (9.2 g, 48.1 mmol) in MeOH (200 mL) in a 500-mL round-bottom flask under nitrogen was added Pd/C (10% wt., 900 mg). The flask was evacuated and flushed three times with hydrogen. The resulting solution was stirred for 12 h at RT under an atmosphere of hydrogen with a balloon. The solids were filtered out. The resulting filtrate was concentrated under vacuum.

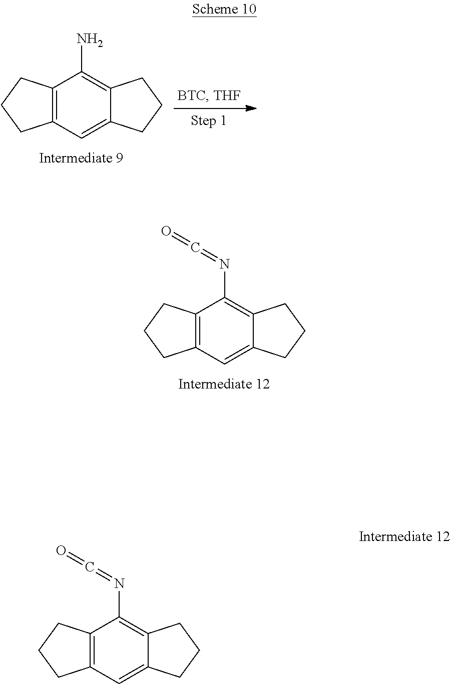 [SMILES: Nc1c2c(cc3c1CCC3)CCC2 ; O=C=Nc1c2c(cc3c1CCC3)CCC2 ; O=C=Nc1c2c(cc3c1CCC3)CCC2]

### 4-Isocyanato-1,2,3,5,6,7-hexahydro-s-indacene

To a stirred solution of 1,2,3,5,6,7-hexahydro-s-indacen-4-amine (64 mg, 0.4 mmol) in THF (5 mL) in a 50-mL round-bottom flask under nitrogen was added BTC (37 mg, 0.1 mmol) in portions at RT. The resulting solution was stirred for 2 h at 65° C. and then was concentrated under vacuum. This resulted in 75 mg (crude) of the title compound as light yellow solid. The crude product was used directly in the next step.

TABLE 3
The Intermediates in the following Table were prepared using the
similar procedures for converting Intermediate 9 to Intermediate 12
shown in Scheme 10 from appropriated starting materials.

| Inter- | | |
|---|---|---|
| mediate # | Structure | IUPAC Name |
| Inter- mediate 13 | 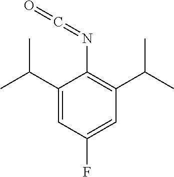 [SMILES: CC(C)c1cc(F)cc(C(C)C)c1N=C=O] | 5-Fluoro-2-isocyanato- 1,3-diisopropylbenzene |
| Inter- mediate 14 | 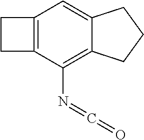 [SMILES: O=C=Nc1c2c(cc3c1CC3)CCC2] | 3-Isocyanato-2,4,5,6- tetrahydro-1H- cyclobuta[f]indene |

Schemes for phenylacetic acids Intermediates: Schemes 11-12 illustrate the coupling of LHS with RHS intermediates.

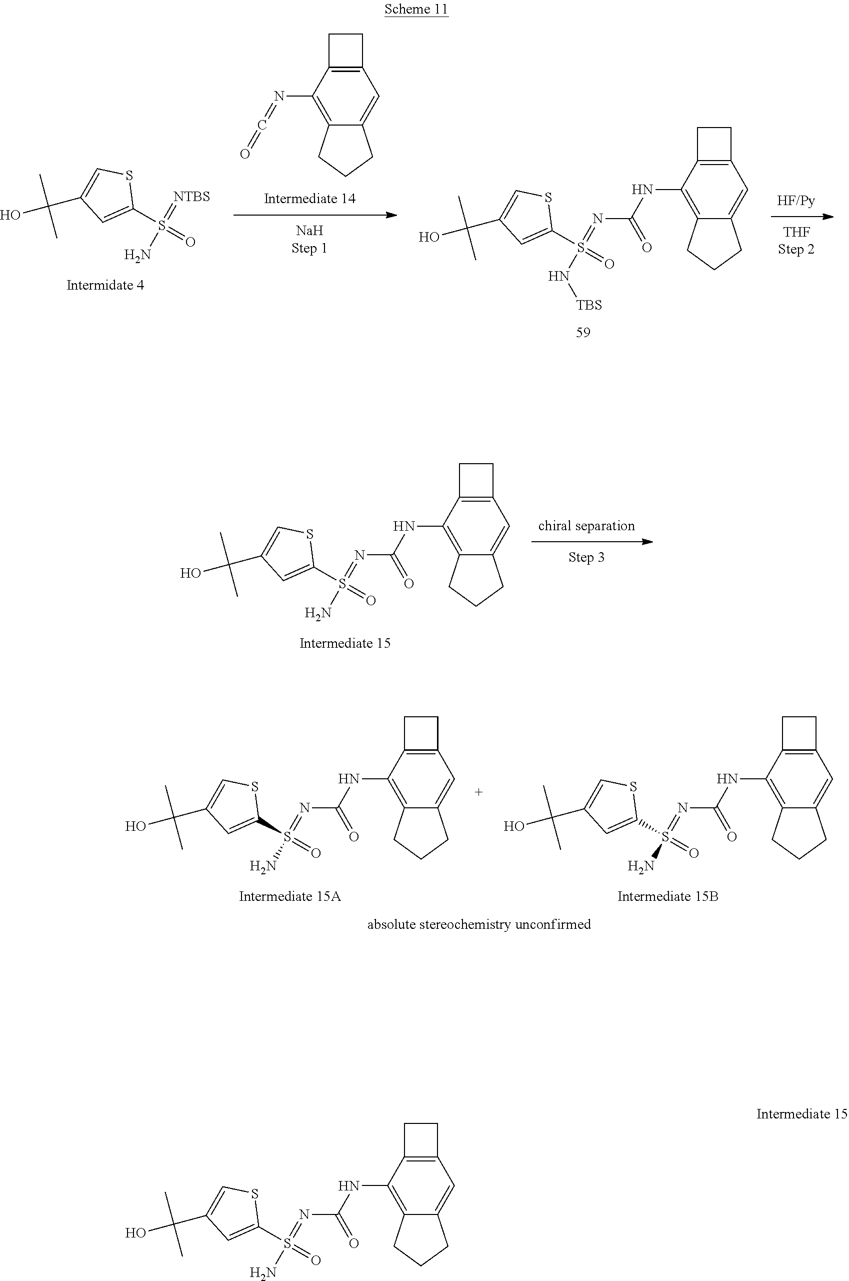

### 4-(2-Hydroxypropan-2-yl)-N′-((2,4,5,6-tetrahydro-1H-cyclobuta[f]inden-3-yl)carbamoyl)thiophene-2-sulfonimidamide

### Intermediate 15A and 15B

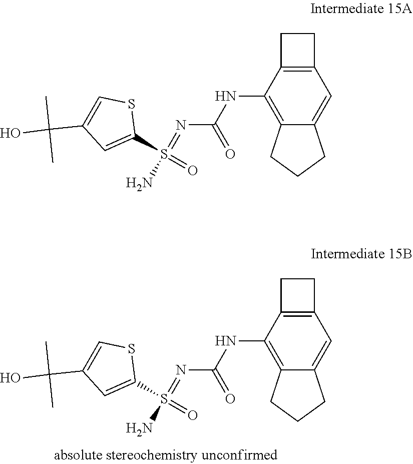 [SMILES: CC(C)(O)c1csc([S@](N)(=O)=NC(=O)Nc2c3c(cc4c2CC4)CCC3)c1 ; CC(C)(O)c1csc([S@@](N)(=O)=NC(=O)Nc2c3c(cc4c2CC4)CCC3)c1]

### (R) and (S)-4-(2-hydroxypropan-2-yl)-N′-((2,4,5,6-tetrahydro-1H-cyclobuta[f]inden-3-yl)carbamoyl)thiophene-2-sulfonimidamide

### Step 1: N-(tert-butyldimethylsilyl)-4-(2-hydroxypropan-2-yl)-N′-((2,4,5,6-tetrahydro-1H-cyclobuta[f]inden-3-yl)carbamoyl)thiophene-2-sulfonimidamide

To a stirred solution of N′-(tert-butyldimethylsilyl)-4-(2-hydroxypropan-2-yl)thiophene-2-sulfonimidamide (1.99 g, 5.95 mmol) in THF (50 mL) in a 100-mL round-bottom flask under nitrogen was added NaH (60% wt. dispersion in mineral oil, 476 mg, 11.9 mmol) in portions at 0° C. in an ice/water bath. The resulting solution was stirred for 20 min at RT. Then 3-isocyanato-2,4,5,6-tetrahydro-1H-cyclobuta[f]indene (1.1 g, 5.95 mmol) in THF (5.0 mL) was added dropwise at 0° C. The reaction solution was stirred for 2 h at RT. Then the reaction solution was quenched by the addition of 5.0 mL of MeOH. The resulting mixture was concentrated. The residue was eluted from silica gel with DCM/MeOH (20:1). This resulted in 3.0 g (97%) of the title compound as a yellow solid. MS-ESI: 520 (M+1).

### Step 2: 4-(2-hydroxypropan-2-yl)-N′-((2,4,5,6-tetrahydro-1H-cyclobuta[f]inden-3-yl)carbamoyl)thiophene-2-sulfonimidamide

To a stirred solution of N-(tert-butyldimethylsilyl)-4-(2-hydroxypropan-2-yl)-N′-((2,4,5,6-tetrahydro-1H- cyclobuta[f]inden-3-yl)carbamoyl)thiophene-2-sulfonimidamide in (3.0 g, 5.77 mmol) in THF (20 mL) in a 100-mL round-bottom flask was added HF-Pyridine (70% wt., 330 mg, 11.5 mmol) at 0° C. in an ice/water bath. The resulting solution was stirred for 30 min at RT. The resulting mixture was concentrated. The residue was eluted from silica gel with DCM/MeOH (30:1). This resulted in 1.8 g (77%) of the title compound as a light yellow solid. MS-ESI: 406 (M+1).

### Step 3: (R) and (S)-4-(2-hydroxypropan-2-yl)-N′-((2,4,5,6-tetrahydro-1H-cyclobuta[f]inden-3-yl) carbamoyl)thiophene-2-sulfonimidamide

The product of 4-(2-hydroxypropan-2-yl)-N′-((2,4,5,6-tetrahydro-1H-cyclobuta[f]inden-3-yl)carbamoyl) thiophene-2-sulfonimidamide (1.8 g, 4.44 mmol) was purified by Prep-SFC with the following conditions: Column, CHIRALPAK IF, 2*25 cm, 5 um; mobile phase, CO₂ (60%) and MeOH (2 mM NH₃—MeOH) (40%); Detector: UV 210/254 nm. This resulted in 750 mg (42%, 99%ee) of Intermediate 15A and 800 mg (44%, 99% ee) of Intermediate 15B both as a white solid. MS-ESI: 406 (M+1).

TABLE 4
The Intermediates in the following table were prepared using similar procedures for
converting Intermeidate 4 to Intermediate 15 shown in Scheme 11 from appropriated starting
materials.

| Intermediate # | Structure | IUPAC Name | Exact Mass [M + H]⁺ |
|---|---|---|---|
| Intermediate 16 | 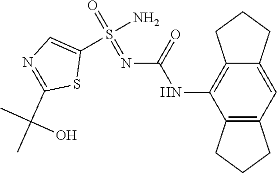 [SMILES: CC(C)(O)c1ncc(S(N)(=O)=NC(=O)Nc2c3c(cc4c2CCC4)CCC3)s1] | N′-((1,2,3,5,6,7-hexahydro-s- indacen-4-yl)carbamoyl)-2-(2- hydroxypropan-2-yl)thiazole- 5-sulfonimidamide | 421 |
| Intermediate 17 | 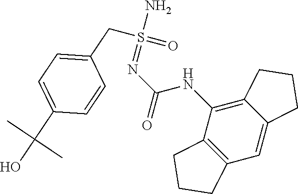 [SMILES: CC(C)(O)c1ccc(CS(N)(=O)=NC(=O)Nc2c3c(cc4c2CCC4)CCC3)cc1] | N′-((1,2,3,5,6,7-hexahydro-s- indacen-4-yl)carbamoyl)-1- (4-(2-hydroxypropan-2- yl)phenyl)methane- sulfonimidamide | 428 |
| Intermediate 18 | 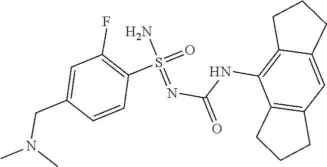 [SMILES: CN(C)Cc1ccc(S(N)(=O)=NC(=O)Nc2c3c(cc4c2CCC4)CCC3)c(F)c1] | 4-((dimethylamino)methyl)-2- fluoro-N′-((1,2,3,5,6,7- hexahydro-s-indacen-4- yl)carbamoyl)benzene- sulfonimidamide | 431 |
| Intermediate 19 | 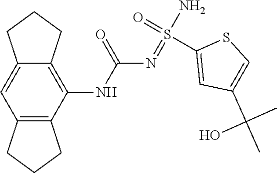 [SMILES: CC(C)(O)c1csc(S(N)(=O)=NC(=O)Nc2c3c(cc4c2CCC4)CCC3)c1] | N′-((1,2,3,5,6,7-hexahydro-s- indacen-4-yl)carbamoyl)-4-(2- hydroxypropan-2-yl)thiophene- 2-sulfonimidamide | 420 |
| Intermediate 20 | 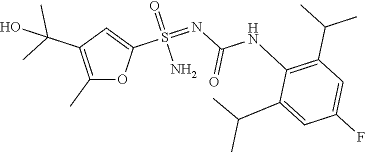 [SMILES: Cc1oc(S(N)(=O)=NC(=O)Nc2c(C(C)C)cc(F)cc2C(C)C)cc1C(C)(C)O] | N′-((4-fluoro-2,6- diisopropylphenyl)carbamoyl)- 4-(2-hydroxypropan-2-yl)-5- methylfuran-2-sulfonimidamide | 440 |

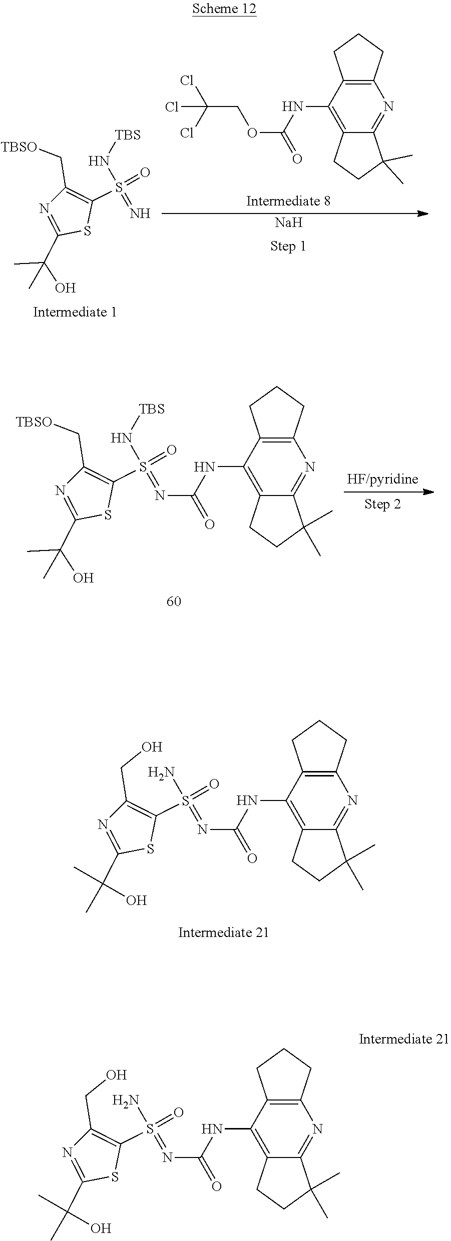

### N-((3,3-dimethyl-1,2,3,5,6,7-hexahydrodicyclopenta[b,e]pyridin-8-yl)carbamoyl)-4-(hydroxymethyl)-2-(2-hydroxypropan-2-yl)thiazole-5-sulfonimidamide

### Step 1: N-(tert-butyldimethylsilyl)-4-((tert-butyldimethylsilyl)methyl)-N′-((3,3-dimethyl-1,2,3,5,6,7-hexahydrodicyclopenta[b,e]pyridin-8-yl)carbamoyl)-2-(2-hydroxypropan-2-yl)thiazole-5-sulfonimidamide

To a stirred solution of N-(tert-butyldimethylsilyl)-4-(((tert-butyldimethylsilyl)oxy)methyl)-2-(2-hydroxypropan-2-yl)thiazole-5-sulfonimidamide (15 g, 31.3 mmol) in THF (100 mL) in a 500-mL round-bottom flask under nitrogen was added NaH (60% wt. dispersion in mineral oil, 3.76 g, 93.9 mmol) in small portions at 0° C. in an ice/water bath. The solution was stirred for 20 min at RT. To the above mixture was added 2,2,2-trichloroethyl (3,3-dimethyl-1,2,3,5,6,7-hexahydrodicyclopenta[b,e]pyridin-8-yl)carbamate (12 g, 31.3 mmol) in small portions at 0° C. The resulting mixture was stirred overnight at RT. The reaction was quenched with 50 mL of water/ice at 0° C. The resulting mixture was extracted with EtOAc (3×100 mL). The combined organic layers were dried over anhydrous Na₂SO₄ and concentrated under vacuum. The residue was eluted from silica gel with DCM/MeOH (20:1). This resulted in 18 g (81%) of the title compound as a yellow oil. MS-ESI: 692 (M+1).

### Step 2: N′4(3,3-dimethyl-1,2,3,5,6,7-hexahydrodicyclopenta[b,e]pyridin-8-yl)carbamoyl)-4-(hydroxymethyl)-2-(2-hydroxypropan-2-yl)thiazole-5-sulfonimidamide

To a stirred solution of N-(tert-butyldimethylsilyl)-4-((tert-butyldimethylsilyl)methyl)-N′-((3,3-dimethyl-1,2,3,5,6,7-hexahydrodicyclopenta[b,e]pyridin-8-yl)carbamoyl)-2-(2-hydroxypropan-2-yl)thiazole-5-sulfonimidamide (18 g, 26 mmol) in THF (50 mL) was added HF-Pyridine (70% wt., 1.49 g, 52 mmol) dropwise at 0° C. in an ice/water bath. The solution was stirred for 1 h at RT. The resulting mixture was concentrated under reduced pressure. The residue was eluted from silica gel with DCM/MeOH (10:1). This resulted in 10.7 g (88%) of the title compound as a yellow solid. MS-ESI: 480 (M+1).

TABLE 5
The Intermediates in the following table were prepared using similar procedures for
converting intermediate 1 to Intermediate 21 shown in Scheme 12 from appropriated starting
materials.

| Intermediate # | Structure | IUPAC Name | Exact Mass [M + H]⁺ |
|---|---|---|---|
| Intermediate 22 | 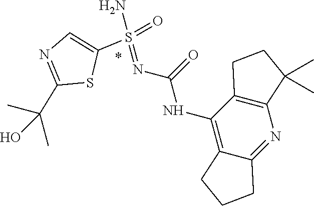 [SMILES: CC(C)(O)c1ncc(S(N)(=O)=NC(=O)Nc2c3c(nc4c2CCC4(C)C)CCC3)s1] | N′-((3,3-dimethyl-1,2,3,5,6,7- hexahydrodicyclopenta[b,e]pyridin- 8-yl)carbamoyl)-2-(2- hydroxypropan-2-yl)thiazole-5- sulfonimidamide | 450 |
| Intermediate 23 | 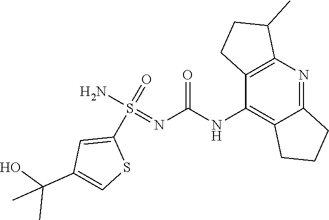 [SMILES: CC1CCc2c1nc1c(c2NC(=O)N=S(N)(=O)c2cc(C(C)(C)O)cs2)CCC1] | 4-(2-hydroxypropan-2-yl)-N′-((3- methyl-1,2,3,5,6,7- hexahydrodicyclopenta[b,e]pyridin- 8-yl)carbamoyl)thiophene-2- sulfonimidamide | 435 |

TABLE 6
The Intermediates in the following table were obtained from chiral HPLC resolutions of
racemic intermediates described above. The chiral column and eluents are listed in the table. As a
convention, the faster-eluting enantiomer is always listed first in the table followed by the slower-
eluting enantiomer of the pair. The symbol * at a chiral center denotes that this chiral center has
been resolved and the absolute stereochemistry at that center has not been determined. Assigned
stereochemistry in compound names are tentative

| | | | | | LCMS |
|---|---|---|---|---|---|
| Ex. # | Structure | IUPAC Name | Column | Eluents | [M + H]⁺ |
| Inter- mediate 17A | 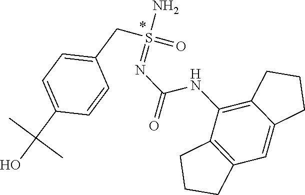 [SMILES: CC(C)(O)c1ccc(CS(N)(=O)=NC(=O)Nc2c3c(cc4c2CCC4)CCC3)cc1] | (S) or (R)-N′-((1,2,3,5,6,7- hexahydro-s-indacen-4- yl)carbamoyl)-1-(4-(2- hydroxypropan-2-yl)phenyl) methanesulfonimidamide | ChiralPak IA, 2*25 cm, 5 um | EtOH in Hex (8 mM NH₃•MeOH)^{#} | 428 |
| Inter- mediate 17B | 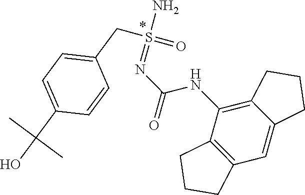 [SMILES: CC(C)(O)c1ccc(CS(N)(=O)=NC(=O)Nc2c3c(cc4c2CCC4)CCC3)cc1] | (R) or (S)-N′-((1,2,3,5,6,7- hexahydro-s-indacen-4- yl)carbamoyl)-1-(4-(2- hydroxypropan-2-yl)phenyl) methanesulfonimidamide | ChiralPak IA, 2*25 cm, 5 um | EtOH in Hex (8 mM NH₃•MeOH)^{#} | 428 |
| Inter- mediate 18A | 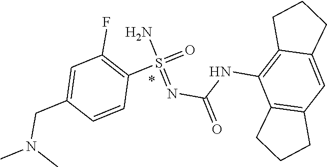 [SMILES: CN(C)Cc1ccc(S(N)(=O)=NC(=O)Nc2c3c(cc4c2CCC4)CCC3)c(F)c1] | (S) or (R)-4- ((dimethylamino)methyl)- 2-fluoro-N′-((1,2,3,5,6,7- hexahydro-s-indacen-4- yl)carbamoyl)benzene- sulfonimidamide | ChiralPak IG, 2*25 cm, 5 um | 30% EtOH in Hex | 431 |
| Inter- mediate 18B | 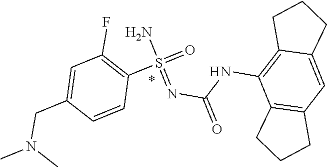 [SMILES: CN(C)Cc1ccc(S(N)(=O)=NC(=O)Nc2c3c(cc4c2CCC4)CCC3)c(F)c1] | (R) or (S)-4- ((dimethylamino)methyl)- 2-fluoro-N′-((1,2,3,5,6,7- hexahydro-s-indacen-4- yl)carbamoyl)benzene- sulfonimidamide | ChiralPak IG, 2*25 cm, 5 um | 30% EtOH in Hex | 431 |
| Inter- mediate 19A | 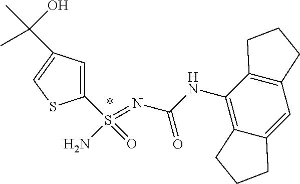 [SMILES: CC(C)(O)c1csc(S(N)(=O)=NC(=O)Nc2c3c(cc4c2CCC4)CCC3)c1] | (S) or (R)-N′-(1,2,3,5,6,7- hexahydro-s-indacen-4- ylcarbamoyl)-4-(2- hydroxypropan-2-yl) thiophene-2-sulfonimi- damide | ChiralPak ID, 2*25 cm, 5 um | 20% EtOH in Hex (0.1% DEA) | 420 |
| Inter- mediate 19B | 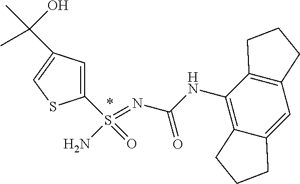 [SMILES: CC(C)(O)c1csc(S(N)(=O)=NC(=O)Nc2c3c(cc4c2CCC4)CCC3)c1] | (R) or (S)-N′-((1,2,3,5,6,7- hexahydro-s-indacen-4- yl)carbamoyl)-4-(2- hydroxypropan-2-yl) thiophene-2- sulfonimidamide | ChiralPak ID, 2*25 cm, 5 um | 20% EtOH in Hex (0.1% DEA) | 420 |
| Inter- mediate 20A | 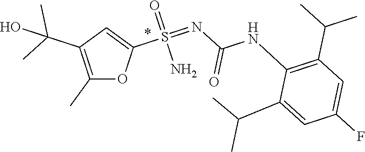 [SMILES: Cc1oc(S(N)(=O)=NC(=O)Nc2c(C(C)C)cc(F)cc2C(C)C)cc1C(C)(C)O] | (S) or (R)-N′-((4-fluoro- 2,6-diisopropylphenyl) carbamoyl)-4-(2- hydroxypropan-2-yl)-5- methylfuran-2-sulfonimi- damide | ChiralPak ID, 0.46*5 cm; 3 um | 20% EtOH in Hex (0.1% DEA) | 440 |
| Inter- mediate 20B | 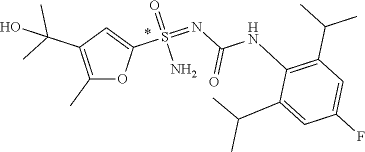 [SMILES: Cc1oc(S(N)(=O)=NC(=O)Nc2c(C(C)C)cc(F)cc2C(C)C)cc1C(C)(C)O] | (R) or (S)-N′-((4-fluoro- 2,6-diisopropylphenyl) carbamoyl)-4-(2- hydroxypropan-2-yl)-5- methylfuran-2-sulfonimi- damide | ChiralPak ID, 0.46*5 cm; 3 um | 20% EtOH in Hex (0.1% DEA) | 440 |
| Inter- mediate 21A | 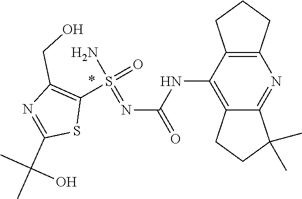 [SMILES: CC(C)(O)c1nc(CO)c(S(N)(=O)=NC(=O)Nc2c3c(nc4c2CCC4(C)C)CCC3)s1] | (S) or (R)-N′-((3,3- dimethyl-1,2,3,5,6,7- hexahydrodicyclopenta [b,e]pyridin-8-yl) carbamoyl)-4- (hydroxymethyl)-2-(2- hydroxypropan-2-yl) thiazole-5-sulfonimi- damide | ChiralPak IG, 2*25 cm, 5 um | 30% MeOH in CO₂ | 480 |
| Inter- mediate 21B | 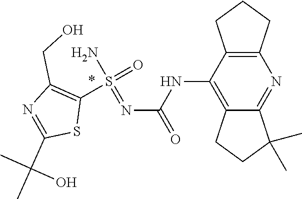 [SMILES: CC(C)(O)c1nc(CO)c(S(N)(=O)=NC(=O)Nc2c3c(nc4c2CCC4(C)C)CCC3)s1] | (R) or (S)-N′-((3,3- dimethyl-1,2,3,5,6,7- hexahydrodicyclopenta [b,e]pyridin-8-yl) carbamoyl)-4- (hydroxymethyl)-2-(2- hydroxypropan-2-yl) thiazole-5-sulfonimi- damide | ChiralPak IG, 2*25 cm, 5 um | 30% MeOH in CO₂ | 480 |
| Inter- mediate 22A | 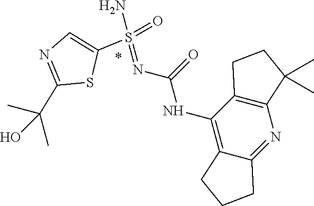 [SMILES: CC(C)(O)c1ncc(S(N)(=O)=NC(=O)Nc2c3c(nc4c2CCC4(C)C)CCC3)s1] | (S) or (R)-N′-((3,3- dimethyl-1,2,3,5,6,7- hexahydrodicyclopenta [b,e]pyridin-8-yl) carbamoyl)-2-(2- hydroxypropan-2-yl) thiazole-5-sulfonimi- damide | CHIRAL ART Cellulose- SB, 2*25 cm, 5 um | EtOH in Hex (8 mM NH₃•MeOH) | 450 |
| Inter- mediate 22B | 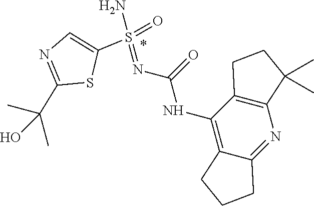 [SMILES: CC(C)(O)c1ncc(S(N)(=O)=NC(=O)Nc2c3c(nc4c2CCC4(C)C)CCC3)s1] | (R) or (S)-N′-((3,3- dimethyl-1,2,3,5,6,7- hexahydrodicyclopenta [b,e]pyridin-8-yl) carbamoyl)-2-(2- hydroxypropan-2-yl) thiazole-5-sulfonimi- damide | CHIRAL ART Cellulose- SB, 2*25 cm, 5 um | EtOH in Hex (8 mM NH₃•MeOH) | 450 |

| | | | | |
|---|---|---|---|---|
| Inter- mediate 23A | 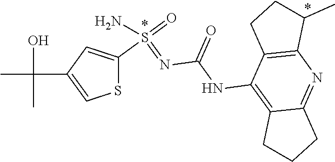 [SMILES: CC1CCc2c1nc1c(c2NC(=O)N=S(N)(=O)c2cc(C(C)(C)O)cs2)CCC1] | (R,R) or (R,S)-4-(2- hydroxypropan-2-yl)- N′-(((S)-3- methyl-1,2,3,5,6,7- hexahydrodicyclopenta [b,e]pyridin-8-yl) carbamoyl)thiophene- 2-sulfonimidamide | Column 2: CHIRALPAK IG, 2*25 cm (5 um) EtOH in MTBE (10 mM NH_{3—} MeOH) to separate two fast-co-eluting isomers from Column 1: Chiralpak IC, 2*25 cm, 5 um, EtOH in Hex (0.1% FA) | 435 |
| Inter- mediate 23B | 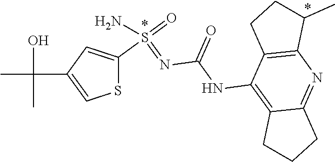 [SMILES: CC1CCc2c1nc1c(c2NC(=O)N=S(N)(=O)c2cc(C(C)(C)O)cs2)CCC1] | (R,S) or (R,R)- 4-(2- hydroxypropan-2-yl)- N′-(((R)-3- methyl-1,2,3,5,6,7- hexahydrodicyclopenta [b,e]pyridin-8-yl) carbamoyl)thiophene- 2-sulfonimidamide | | 435 |
| Inter- mediate 23C | 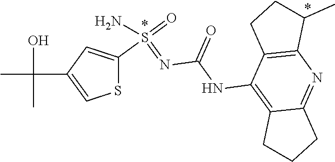 [SMILES: CC1CCc2c1nc1c(c2NC(=O)N=S(N)(=O)c2cc(C(C)(C)O)cs2)CCC1] | (S,S) or (S,R)-4-(2- hydroxypropan-2-yl)- N′-(((S)-3- methyl-1,2,3,5,6,7- hexahydrodicyclopenta [b,e]pyridin-8-yl) carbamoyl)thiophene- 2-sulfonimidamide | Column 2: CHIRALPAK IG, 2*25 cm (5 um) EtOH in MTBE (10 mM NH_{3—}MeOH) to separate two slow-co-eluting isomers from Column 1: Chiralpak IC, 2*25 cm, 5 um, EtOH in Hex (0.1% FA) | 435 |
| Inter- mediate 23D | 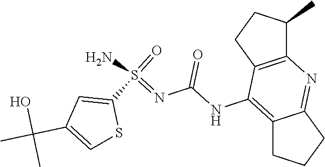 [SMILES: C[C@@H]1CCc2c1nc1c(c2NC(=O)N=[S@@](N)(=O)c2cc(C(C)(C)O)cs2)CCC1] | (S,R) or (S,S)-4-(2- hydroxypropan-2-yl)- N′-(((R)-3- methyl-1,2,3,5,6,7- hexahydrodicyclopenta [b,e]pyridin-8-yl) carbamoyl)thiophene- 2-sulfonimidamide | | 435 |

^{#}Concentration of NH₃ obtained after adding 2 M NH₃•MeOH (used in other similar entries)

### Example 1 (Compound 105a)

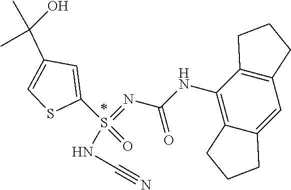 [SMILES: CC(C)(O)c1csc(S(=O)(=NC(=O)Nc2c3c(cc4c2CCC4)CCC3)NC#N)c1]

### (S) or (R)-N-cyano-N′-(1,2,3,5,6,7-hexahydro-s-indacen-4-ylcarbamoyl)-4-(2-hydroxypropan-2-yl)thiophene-2-sulfonimidamide (Scheme I)

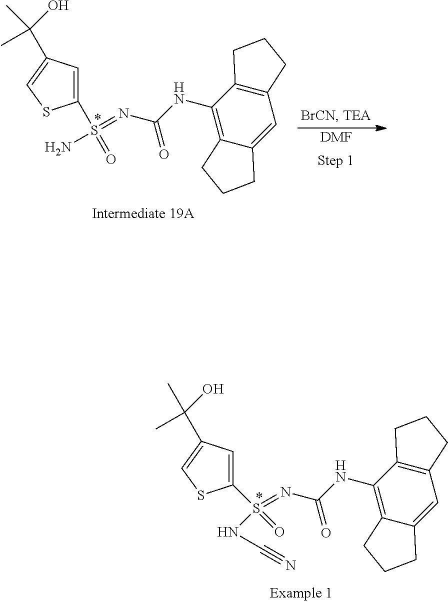

To a stirred solution of (S) or (R)-N′-(1,2,3,5,6,7-hexahydro-s-indacen-4-ylcarbamoyl)-4-(2-hydroxypropan-2-yl)thiophene-2-sulfonimi damide (Intermediate 19A, 200 mg, 0.48 mmol) in DMF (10 mL) in a 50 mL round-bottom flask was added TEA (193 mg, 1.91 mmol) at RT, followed by the addition of cyanogen bromide (101 mg, 0.95 mmol) in portions at RT. The resulting solution was stirred for 4 h at RT. The pH value of the solution was adjusted to 10 with NaOH (1 M). The resulting mixture was concentrated under vacuum. The crude product was purified by Flash-Prep-HPLC with the following conditions: XSelect CSH Prep C18 OBD Column, 19*150 mm; 5 um, Mobile Phase A: water (10 mM NH₄HCO₃+0.1% NH₃H₂O), Mobile Phase B: MeCN; Flow rate: 25 mL/min; Gradient: 25% B to 33% B over 7 min; UV 254/220 nm; Rt₁: 6.20 min. This resulted in 38 mg (18%) of Example 1 as a white solid. MS-ESI: 445 (M+1). ¹H NMR (300 MHz, DMSO-d₆) δ 7.98 (br s, 1H), 7.45 (d, J=1.8 Hz, 1H), 7.41 (d, J=1.8 Hz, 1H), 7.08 (br s, 1H), 6.82 (s, 1H), 5.14 (s, 1H), 2.79-2.70 (m, 4H), 2.69-2.61 (m, 4H), 1.96-1.82 (m, 4H), 1.42 (s, 6H).

TABLE 7
Examples in the following table were prepared using similar conditions as described in
Example 1 and Scheme I from appropriate starting materials.

| | From | Final | | | Exact |
|---|---|---|---|---|---|
| Ex. | Int. | Target | | | Mass |
| # | # | Number | Structure | IUPAC Name | [M + H]⁺ |
| 2 | 19B | 105b | 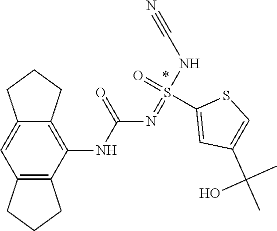 [SMILES: CC(C)(O)c1csc(S(=O)(=NC(=O)Nc2c3c(cc4c2CCC4)CCC3)NC#N)c1] | (R) or (S)-N- cyano-N′- ((1,2,3,5,6,7- hexahydro-s- indacen-4- yl)carbamoyl)-4-(2- hydroxypropan-2- yl)thiophene-2- sulfonimidamide | 445 |
| 3 | 16 | 106 | 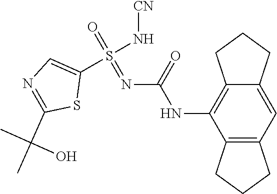 [SMILES: CC(C)(O)c1ncc(S(=O)(=NC(=O)Nc2c3c(cc4c2CCC4)CCC3)NC#N)s1] | N-cyano-N′- ((1,2,3,5,6,7- hexahydro-s- indacen-4- yl)carbamoyl)-2-(2- hydroxypropan-2- yl)thiazole-5- sulfonimidamide | 446 |
| 4 | 22A | 104b | 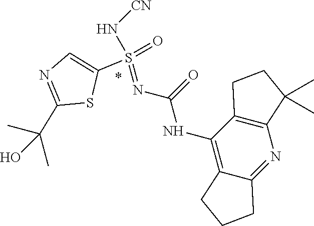 [SMILES: CC(C)(O)c1ncc(S(=O)(=NC(=O)Nc2c3c(nc4c2CCC4(C)C)CCC3)NC#N)s1] | (S) or (R)-N- cyano-N′-((3,3- dimethyl- 1,2,3,5,6,7- hexahydrodicyclo- penta[b,e]pyridin- 8-yl)carbamoyl)-2- (2-hydroxypropan- 2-yl)thiazole-5- sulfonimidamide | 475 |
| 5 | 22B | 104a | 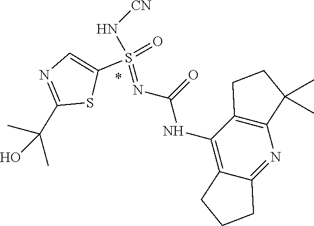 [SMILES: CC(C)(O)c1ncc(S(=O)(=NC(=O)Nc2c3c(nc4c2CCC4(C)C)CCC3)NC#N)s1] | (R) or (S)-N- cyano-N′-((3,3- dimethyl- 1,2,3,5,6,7- hexahydrodicyclo- penta[b,e]pyridin- 8-yl)carbamoyl)-2- (2-hydroxypropan- 2-yl)thiazole-5- sulfonimidamide | 475 |
| 6 | 15A | 103b | 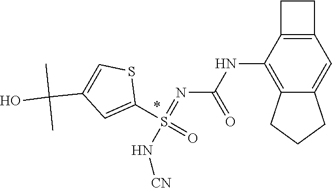 [SMILES: CC(C)(O)c1csc(S(=O)(=NC(=O)Nc2c3c(cc4c2CC4)CCC3)NC#N)c1] | (R) or (S)-N- cyano-4-(2- hydroxypropan-2- yl)-N′-((2,4,5,6- tetrahydro-1H- cyclobuta[f]inden- 3-yl)carbamoyl) thiophene-2- sulfonimidamide | 431 |
| 7 | 15B | 103a | 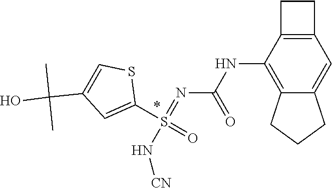 [SMILES: CC(C)(O)c1csc(S(=O)(=NC(=O)Nc2c3c(cc4c2CC4)CCC3)NC#N)c1] | (S) or (R)-N- cyano-4-(2- hydroxypropan-2- yl)-N′-((2,4,5,6- tetrahydro-1H- cyclobuta[f] inden-3- yl)carbamoyl) thiophene-2- sulfonimidamide | 431 |
| 8 | 17A | 102b | 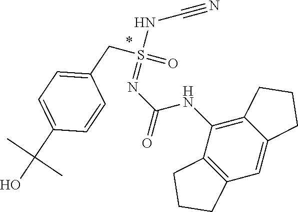 [SMILES: CC(C)(O)c1ccc(CS(=O)(=NC(=O)Nc2c3c(cc4c2CCC4)CCC3)NC#N)cc1] | (S) or (R)-N- cyano-N′- ((1,2,3,5,6,7- hexahydro-s- indacen-4- yl)carbamoyl)- 1-(4-(2- hydroxypropan- 2-yl)phenyl) methane- sulfonimidamide | 453 |
| 9 | 17B | 102a | 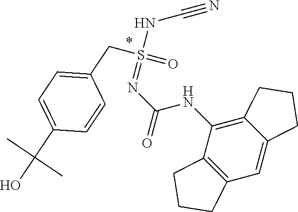 [SMILES: CC(C)(O)c1ccc(CS(=O)(=NC(=O)Nc2c3c(cc4c2CCC4)CCC3)NC#N)cc1] | (R) or (S)-N- cyano-N′- ((1,2,3,5,6,7- hexahydro-s- indacen-4- yl)carbamoyl)- 1-(4-(2- hydroxypropan- 2-yl)phenyl) methane- sulfonimidamide | 453 |
| 10 | 20A | 101b | 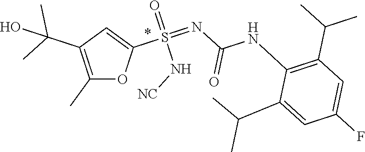 [SMILES: Cc1oc(S(=O)(=NC(=O)Nc2c(C(C)C)cc(F)cc2C(C)C)NC#N)cc1C(C)(C)O] | (S) or (R)-N- cyano-N′-((4- fluoro-2,6- diisopropyl- phenyl) carbamoyl)-4-(2- hydroxypropan- 2-yl)-5- methylfuran-2- sulfonimidamide | 465 |
| 11 | 20B | 101a | 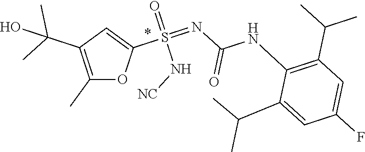 [SMILES: Cc1oc(S(=O)(=NC(=O)Nc2c(C(C)C)cc(F)cc2C(C)C)NC#N)cc1C(C)(C)O] | (R) or (S)-N- cyano-N′-((4- fluoro-2,6- diisopropyl- phenyl) carbamoyl)-4-(2- hydroxypropan- 2-yl)-5- methylfuran-2- sulfonimidamide | 465 |
| 12 | 21A | 108a | 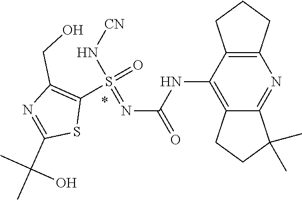 [SMILES: CC(C)(O)c1nc(CO)c(S(=O)(=NC(=O)Nc2c3c(nc4c2CCC4(C)C)CCC3)NC#N)s1] | (S) or-N-cyano- N′-((3,3-dimethyl- 1,2,3,5,6,7- hexahydrodicyclo- penta[b,e]pyridin- 8-yl)carbamoyl)- 4-(hydroxy- methyl)-2-(2- hydroxypropan- 2-yl)thiazole-5- sulfonimidamide | 505 |
| 13 | 21B | 108b | 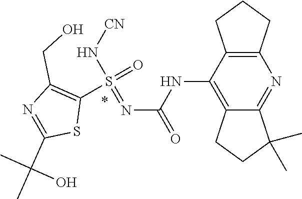 [SMILES: CC(C)(O)c1nc(CO)c(S(=O)(=NC(=O)Nc2c3c(nc4c2CCC4(C)C)CCC3)NC#N)s1] | (R) or (S)-N- cyano-N′-((3,3- dimethyl- 1,2,3,5,6,7- hexahydrodicyclo- penta[b,e]pyridin- 8-yl)carbamoyl)- 4-(hydroxy- methyl)-2-(2- hydroxypropan- 2-yl)thiazole-5- sulfonimidamide | 505 |
| 14 | 18A | 110a | 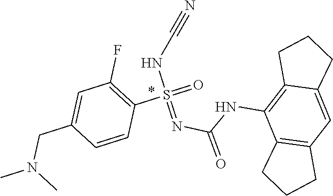 [SMILES: CN(C)Cc1ccc(S(=O)(=NC(=O)Nc2c3c(cc4c2CCC4)CCC3)NC#N)c(F)c1] | (S) or (R)-N- cyano-4- ((dimethylamino) methyl)-2-fluoro- N′-((1,2,3,5,6,7- hexahydro-s- indacen-4- yl)carbamoyl) benzene- sulfonimidamide | 456 |
| 15 | 18B | 110b | 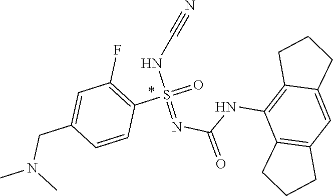 [SMILES: CN(C)Cc1ccc(S(=O)(=NC(=O)Nc2c3c(cc4c2CCC4)CCC3)NC#N)c(F)c1] | (R) or (S)-N- cyano-4- ((dimethylamino) methyl)-2-fluoro- N′-((1,2,3,5,6,7- hexahydro-s- indacen-4- yl)carbamoyl) benzene- sulfonimidamide | 456 |
| 16 | 23A | 109a | 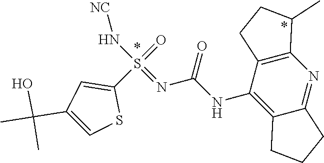 [SMILES: CC1CCc2c1nc1c(c2NC(=O)N=S(=O)(NC#N)c2cc(C(C)(C)O)cs2)CCC1] | (R,R) or (R,S)- N-cyano-4-(2- hydroxypropan- 2-yl)-N′-(((S)- 3-methyl- 1,2,3,5,6,7- hexahydro- dicyclopenta [b,e]pyridin-8- yl)carbamoyl) thiophene-2- sulfonimidamide | 460 |
| 17 | 23B | 109b | 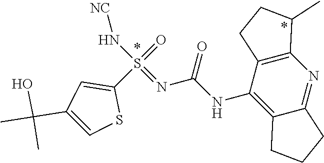 [SMILES: CC1CCc2c1nc1c(c2NC(=O)N=S(=O)(NC#N)c2cc(C(C)(C)O)cs2)CCC1] | (R,S) or (R,R)- N-cyano-4-(2- hydroxypropan- 2-yl)-N′-(((R)- 3-methyl- 1,2,3,5,6,7- hexahydro- dicyclopenta [b,e]pyridin-8- yl)carbamoyl) thiophene-2- sulfonimidamide | 460 |
| 18 | 23C | 109c | 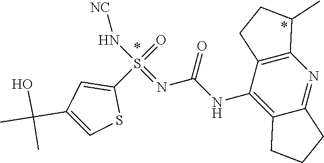 [SMILES: CC1CCc2c1nc1c(c2NC(=O)N=S(=O)(NC#N)c2cc(C(C)(C)O)cs2)CCC1] | (S,S) or (S,R)- N-cyano-4-(2- hydroxypropan- 2-yl)-N′-(((S)- 3-methyl- 1,2,3,5,6,7- hexahydro- dicyclopenta [b,e]pyridin-8- yl)carbamoyl) thiophene-2- sulfonimidamide | 460 |
| 19 | 23D | 109d | 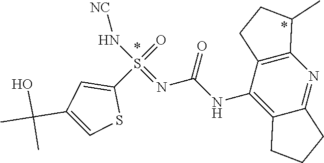 [SMILES: CC1CCc2c1nc1c(c2NC(=O)N=S(=O)(NC#N)c2cc(C(C)(C)O)cs2)CCC1] | (S,R) or (S,S)- N-cyano-4-(2- hydroxypropan- 2-yl)-N′-(((R)- 3-methyl- 1,2,3,5,6,7- hexahydro- dicyclopenta [b,e]pyridin-8- yl)carbamoyl) thiophene-2- sulfonimidamide | 460 |

Schemes for Sulfonimidoylamide Intermediates: Schemes below illustrate the preparation of sulfonimidamide and aniline intermediates.

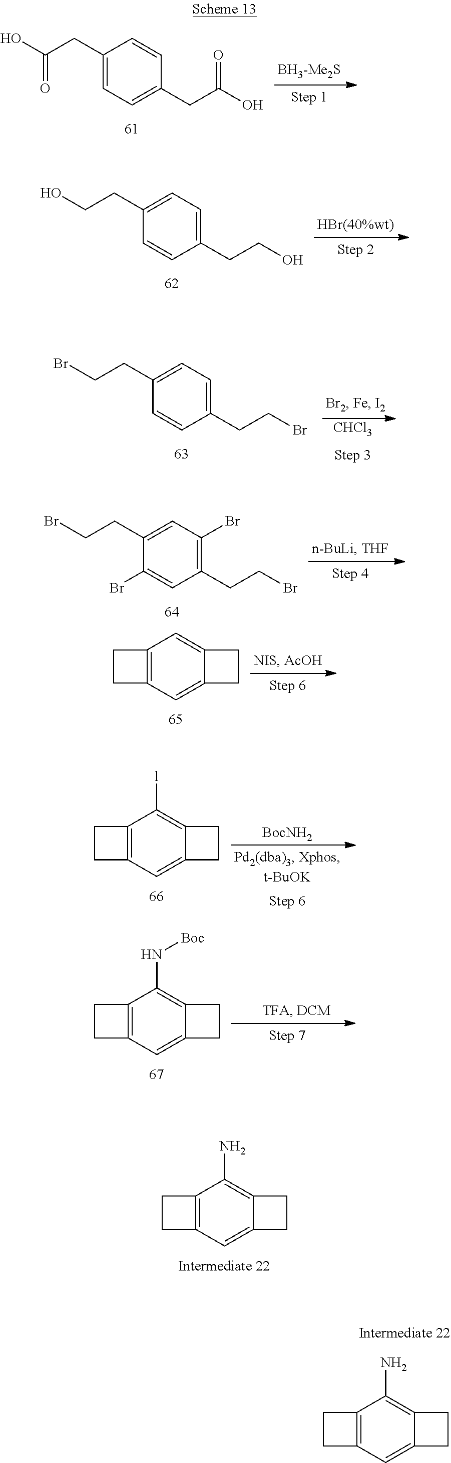

### Tricyclo[6.2.0.0^{3,6}]deca-1,3(6),7-trien-2-amine

### Step 1: 2,2′-(1,4-Phenylene)bis(ethan-1-ol)

To a stirred solution of 2,2′-(1,4-phenylene)diacetic acid (40 g, 200 mmol) in THF (500 mL) under nitrogen was added BH₃—Me₂S (60 mL, 600 mmol, 10 M) dropwise at 0° C. The resulting solution was stirred for 24 h at RT. The reaction was then quenched with 200 mL of water and extracted with 2×150 mL of EtOAc. The organic layers were combined and dried over anhydrous Na₂SO₄ and concentrated under vacuum. The residue was eluted from a silica gel column with EtOAc/PE (1:10 to 1:3). This resulted in 28 g (81.8%) of the title compound as brown oil. MS-ESI: 167 (M+1).

### Step 2: 1,4-Bis(2-bromoethyl)benzene

### The solution of 2,2′-(1,4-phenylene)bis(ethan-1-ol) (28 g, 168 mmol) in aq. HBr (300 mL, 40% wt.) was stirred for 5 h at 100° C. The resulting solution was diluted with 500 mL of water and extracted with 3×200 mL of DCM. The organic layers were combined and dried over anhydrous Na₂SO₄, then concentrated under vacuum. This resulted in 40 g (81.4%) of the title compound as a white solid. MS-ESI: 291/293/295 (M+1).

### Step 3: 1,4-Dibromo-2,5-bis(2-bromoethyl)benzene

To a stirred solution of 1,4-bis(2-bromoethyl)benzene (30 g, 103 mmol) in CHCl₃ (200 mL) was added 12 (0.78 g, 3.08 mmol), iron powder (0.75 g, 13.4 mmol) and Br₂ (41 g, 257 mmol) at RT. The resulting solution was stirred for 24 h at RT. The reaction was then quenched with 200 mL of sat. aqueous Na₂SO₃ and extracted with 3×200 mL of DCM. The organic layers were combined and dried over anhydrous Na₂SO₄ and concentrated under vacuum. This resulted in 40 g (86.6%) of the title compound as a white solid. MS-ESI: 449/451/453 (M+1).

### Step 4: Tricyclo[6.2.0.0^{3,6}]deca-1,3(6),7-triene

To a stirred solution of 1,4-dibromo-2,5-bis(2-bromoethyl)benzene (40 g, 88.9 mmol) in THF (400 mL) under nitrogen was added n-BuLi (74.7 mL, 187 mmol, 2.5 M) dropwise at −78° C. The resulting solution was stirred for 30 min at −78° C. The reaction was then quenched with sat. aqueous NH₄Cl (300 ml) and extracted with 2×200 mL of DCM. The organic layers were combined and dried over anhydrous Na₂SO₄ and concentrated under vacuum. This resulted in 8.0 g (69.1%) of the title compound as a light yellow solid. GC-MS: 130.

### Step 5: 2-Iodotricyclo[6.2.0.0^{3,6}]deca-1,3(6),7-triene

To a stirred solution of tricyclo[6.2.0.0^{3,6}]deca-1,3(6),7-triene (8 g, 61.45 mmol) in AcOH (50 mL) was added NIS (20.7 g, 92.2 mmol) in portions at RT. The resulting solution was stirred for 3 h at 50° C. The resulting solution was diluted with 100 mL of water and then quenched with 50 mL of sat. aqueous Na₂SO₃. The resulting solution was extracted with 3×50 mL of DCM. The organic layers were combined and dried over anhydrous Na₂SO₄ and concentrated under vacuum. The residue was eluted from a silica gel column with EtOAc/PE (1:100). This resulted in 2.5 g (18.2%) of the title compound as a white solid. GC-MS: 256.

### Step 6: Tert-butyl tricyclo[6.2.0.0^{3,6}]deca-1,3(6),7-trien-2-ylcarbamate

To a stirred solution of 2-iodotricyclo[6.2.0.0^{3,6}]deca-1,3(6),7-triene (2.5 g, 9.76 mmol) in toluene (50 mL) under nitrogen was added tert-butyl carbamate (3.43 g, 29.3 mmol), Pd₂(dba)₃ (447 mg, 0.49 mmol), Xphos (466 mg, 0.98 mmol) and t-BuOK (3.29g, 29.3 mmol). The resulting solution was stirred for 14 h at 100° C. The resulting mixture was concentrated under vacuum. The residue was eluted from a silica gel column with EtOAc/PE (1:30). This resulted in 1.5 g (62.6%) of the title compound as a light yellow solid. MS-ESI: 246 (M+1).

### Step 7: Tricyclo[6.2.0.0^{3,6}]deca-1,3(6),7-trien-2-amine

To a stirred solution of tert-butyl N-[tricyclo[6.2.0.0^{3,6}]deca-1,3(6),7-trien-2-yl]carbamate (1.5 g, 6.1 mmol) in DCM (20 mL) was added TFA (4 mL). The resulting solution was stirred for 2 h at RT and then concentrated under vacuum. This resulted in 800 mg (crude) of the title compound as a brown solid. MS-ESI: 146 (M+1).

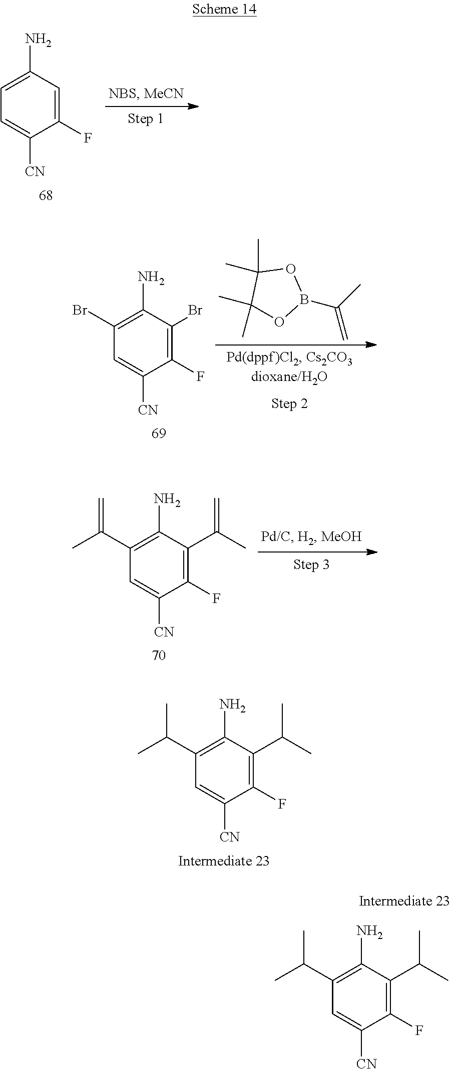

### 4-Amino-2-fluoro-3,5-diisopropylbenzonitrile

### Step 1: 4-Amino-3,5-dibromo-2-fluorobenzonitrile

To a stirred solution of 4-amino-2-fluorobenzonitrile (25 g, 184 mmol) in ACN (500 mL) under nitrogen was added NBS (81.7 g, 459 mmol) in portions at RT. The resulting solution was stirred for 16 hr at 85° C. The resulting mixture was cooled to 0° C. and quenched with 3×100 ml of water. The resulting solution was extracted with 3×100 mL of DCM. The residue was eluted from a silica gel column with EtOAc/PE (1:10). This resulted in 45 g (83.3%) of the title compound as a brown solid. MS-ESI: 293, 295, 297 (M+1).

Steps 2-3 used similar procedures for converting compound 57 to Intermediate 11 shown in Scheme 9 to afford Intermediate 23 from compound 68. MS-ESI: 221 (M+1).

TABLE 8
The Intermediate in the following Table were prepared using the
similar procedures for converting compound 68 to Intermediate
23 shown in Scheme 14 from appropriate reagents.

| Inter- | | | Exact |
|---|---|---|---|
| mediate | | | Mass |
| # | Structure | IUPAC Name | [M + H]⁺ |
| Inter- mediate 24 | 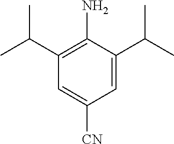 | 4-Amino-3,5- diisopropyl- benzonitrile | 203 |

TABLE 3A
The Intermediates in the following Table were prepared using the
similar procedures for converting Intermediate 9 to Intermediate
12 shown in Scheme 10 from appropriated starting materials.

| Inter- | | |
|---|---|---|
| mediate # | Structure | IUPAC Name |
| Inter- mediate 25 | 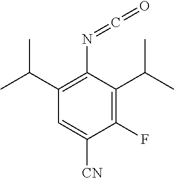 [SMILES: CC(C)c1cc(C#N)c(F)c(C(C)C)c1N=C=O] | 2-Fluoro-4- isocyanato-3,5- diisopropyl- benzonitrile |
| Inter- mediate 26 | 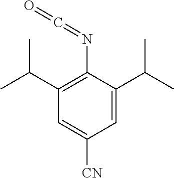 [SMILES: CC(C)c1cc(C#N)cc(C(C)C)c1N=C=O] | 4-Isocyanato- 3,5-diisopropyl- benzonitrile |

TABLE 4A
The Intermediates in the following table were prepared using similar procedures for
converting Intermeidate 4 to Intermediate 15 shown in Scheme 11 from appropriated starting
materials.

| Intermediate # | Structure | IUPAC Name | Exact Mass [M + H]⁺ |
|---|---|---|---|
| Intermediate 27 | 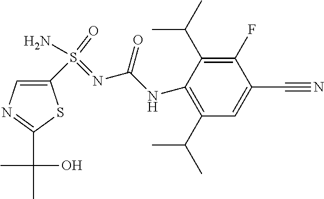 [SMILES: CC(C)c1cc(C#N)c(F)c(C(C)C)c1NC(=O)N=S(N)(=O)c1cnc(C(C)(C)O)s1] | N′-((4-cyano-3-fluoro- 2,6-diisopropylphenyl) carbamoyl)-2-(2- hydroxypropan-2-yl) thiazole-5- sulfonimidamide | 468 |
| Intermediate 28 | 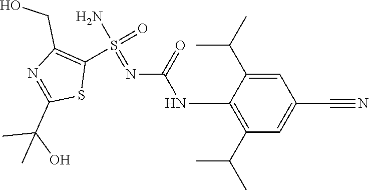 [SMILES: CC(C)c1cc(C#N)cc(C(C)C)c1NC(=O)N=S(N)(=O)c1sc(C(C)(C)O)nc1CO] | N′-((4-cyano-2,6- diisopropylphenyl) carbamoyl)-4- (hydroxymethyl)-2- (2-hydroxypropan- 2-yl)thiazole-5- sulfonimidamide | 480 |
| Intermediate 29 | 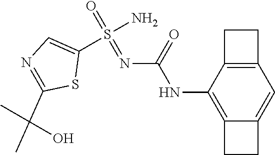 [SMILES: CC(C)(O)c1ncc(S(N)(=O)=NC(=O)Nc2c3c(cc4c2CC4)CC3)s1] | 2-(2-Hydroxy- propan-2-yl)-N′- (tricyclo[6.2.0.03,6] deca-1,3(6),7- trien-2-ylcarbamoyl) thiazole-5- sulfonimidamide | 393 |
| Intermediate 30 | 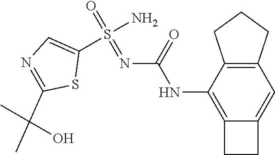 [SMILES: CC(C)(O)c1ncc(S(N)(=O)=NC(=O)Nc2c3c(cc4c2CC4)CCC3)s1] | 2-(2-Hydroxy- propan-2-yl)-N′- ((2,4,5,6-tetrahydro- 1H-cyclobuta[f] inden-3-yl) carbamoyl) thiazole-5- sulfonimidamide | 407 |
| Intermediate 31 | 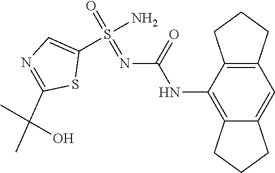 [SMILES: CC(C)(O)c1ncc(S(N)(=O)=NC(=O)Nc2c3c(cc4c2CCC4)CCC3)s1] | N′-((1,2,3,5,6,7- hexahydro-s- indacen-4-yl) carbamoyl)-2-(2- hydroxypropan- 2-yl)thiazole-5- sulfonimidamide | 421 |

TABLE 6A
The Intermediates in the following table were obtained from chiral HPLC resolutions
of racemic intermediates described above. The chiral column and eluents are listed in the table.
As a convention, the faster-eluting enantiomer is always listed first in the table followed by the
slower-eluting enantiomer of the pair. The symbol * at a chiral center denotes that this chiral
center has been resolved and the absolute stereochemistry at that center has not been determined.
Assigned stereochemistry in compound names are tentative.

| Inter- | | | | | |
|---|---|---|---|---|---|
| mediate | | | | | LC-MS |
| # | Structure | IUPAC Name | Column | Eluents | [M + H]⁺ |
| Inter- mediate 27A | 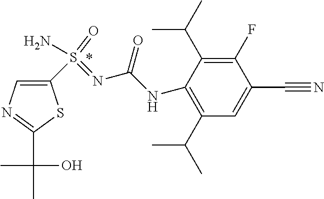 [SMILES: CC(C)c1cc(C#N)c(F)c(C(C)C)c1NC(=O)N=S(N)(=O)c1cnc(C(C)(C)O)s1] | (R) or (S)-N′-((4-cyano- 3-fluoro-2,6-diisopropyl- phenyl)carbamoyl)-2-(2- hydroxypropan-2-yl) thiazole-5-sulfonimi- damide | CHIRAL- PAK ID, 0.46*5 cm; 3 um | 50% IPA in Hex (0.1% DEA) | 468 |
| Inter- mediate 27B | 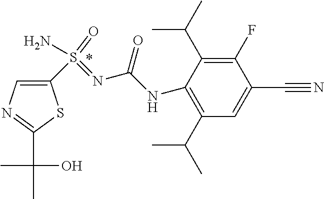 [SMILES: CC(C)c1cc(C#N)c(F)c(C(C)C)c1NC(=O)N=S(N)(=O)c1cnc(C(C)(C)O)s1] | (S) or (R)-N′-((4-cyano- 3-fluoro-2,6-diisopropyl- phenyl)carbamoyl)-2-(2- hydroxypropan-2-yl) thiazole-5-sulfonimi- damide | CHIRAL- PAK ID, 0.46*5 cm; 3 um | 50% IPA in Hex (0.1% DEA) | 468 |
| Inter- mediate 28A | 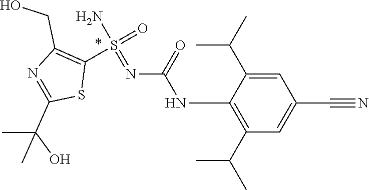 [SMILES: CC(C)c1cc(C#N)cc(C(C)C)c1NC(=O)N=S(N)(=O)c1sc(C(C)(C)O)nc1CO] | (R) or (S)-N′-((4-cyano- 2,6-diisopropylphenyl) carbamoyl)-4- (hydroxymethyl)-2-(2- hydroxypropan-2-yl) thiazole-5-sulfonimi- damide | CHIRAL- PAK IC, 0.46*5 cm; 3 um | 15% EtOH in Hex | 480 |
| Inter- mediate 28B | 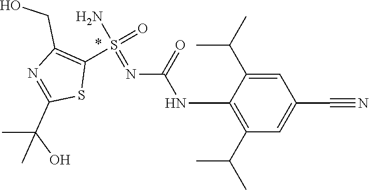 [SMILES: CC(C)c1cc(C#N)cc(C(C)C)c1NC(=O)N=S(N)(=O)c1sc(C(C)(C)O)nc1CO] | (S) or (R)-N′-((4-cyano- 2,6-diisopropylphenyl) carbamoyl)-4- (hydroxymethyl)-2-(2- hydroxypropan-2-yl) thiazole-5-sulfonimi- damide | CHIRAL- PAK IC, 0.46*5 cm; 3 um | 15% EtOH in Hex | 480 |
| Inter- mediate 29A | 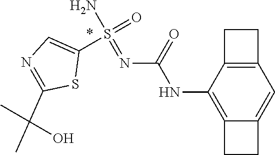 [SMILES: CC(C)(O)c1ncc(S(N)(=O)=NC(=O)Nc2c3c(cc4c2CC4)CC3)s1] | (R) or (S)-2-(2- hydroxypropan-2-yl)- N′-(tricyclo[6.2.0.03,6] deca-1,3(6),7-trien-2- ylcarbamoyl)thiazole- 5-sulfonimidamide | Chiralpak ID, 2*25 cm, 5 um | 20% IPA in Hex (0.1% FA) | 393 |
| Inter- mediate 29B | 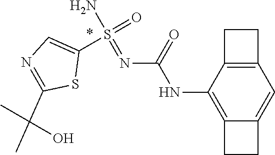 [SMILES: CC(C)(O)c1ncc(S(N)(=O)=NC(=O)Nc2c3c(cc4c2CC4)CC3)s1] | (S) or (R)-2-(2- hydroxypropan-2-yl)- N′-(tricyclo[6.2.0.03,6] deca-1,3(6),7-trien-2- ylcarbamoyl)thiazole- 5-sulfonimidamide | Chiralpak ID, 2*25 cm, 5 um | 20% IPA in Hex (0.1% FA) | 393 |
| Inter- mediate 30A | 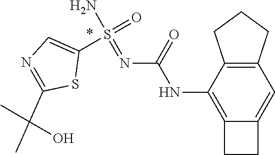 [SMILES: CC(C)(O)c1ncc(S(N)(=O)=NC(=O)Nc2c3c(cc4c2CC4)CCC3)s1] | (R) or (S)-2-(2- hydroxypropan-2-yl)- N′-((2,4,5,6- tetrahydro-1H- cyclobuta[f]inden-3- yl)carbamoyl) thiazole-5- sulfonimidamide | CHIRAL- PAK IG, 20*250 mm, 5 um | 30% EtOH in Hex (0.1% FA) | 407 |
| Inter- mediate 30B | 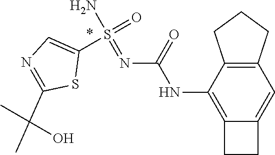 [SMILES: CC(C)(O)c1ncc(S(N)(=O)=NC(=O)Nc2c3c(cc4c2CC4)CCC3)s1] | (S) or (R)-2-(2- hydroxypropan-2-yl)- N′-((2,4,5,6- tetrahydro-1H- cyclobuta[f]inden-3- yl)carbamoyl) thiazole-5- sulfonimidamide | CHIRAL- PAK IG, 20*250 mm, 5 um | 30% EtOH in Hex (0.1% FA) | 407 |
| Inter- mediate 31A | 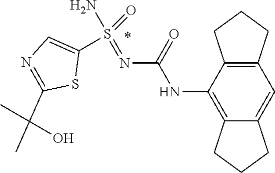 [SMILES: CC(C)(O)c1ncc(S(N)(=O)=NC(=O)Nc2c3c(cc4c2CCC4)CCC3)s1] | (R) or (S)-N′- ((1,2,3,5,6,7- hexahydro-s-indacen- 4-yl)carbamoyl)-2- (2-hydroxypropan-2- yl)thiazole-5- sulfonimidamide | CHIRAL- PAK IF, 2*25 cm, 5 um | 40% MeOH (2 mM NH_{3—}MeOH) in CO₂ | 421 |
| Inter- mediate 31B | 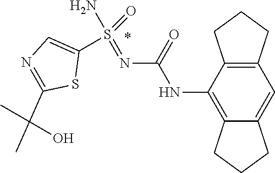 [SMILES: CC(C)(O)c1ncc(S(N)(=O)=NC(=O)Nc2c3c(cc4c2CCC4)CCC3)s1] | (S) or (R)-N′- ((1,2,3,5,6,7- hexahydro-s-indacen- 4-yl)carbamoyl)-2- (2-hydroxypropan-2- yl)thiazole-5- sulfonimidamide | CHIRAL- PAK IF, 2*25 cm, 5 um | 40% MeOH (2 mM NH_{3—}MeOH) in CO₂ | 421 |

TABLE 7A
Examples in the following table were prepared using similar conditions as described
in Example 1 and Scheme I from appropriate starting materials described above.

| Ex. | From | | | Exact Mass |
|---|---|---|---|---|
| # | Int. # | Structure | IUPAC Name | [M + H]⁺ |
| 20 | 27A | 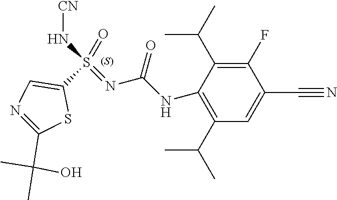 | (S) or (R)-N-cyano-N′-((4-cyano-3- fluoro-2,6- diisopropylphenyl)carbamoyl)-2- (2-hydroxypropan-2-yl)thiazole-5- sulfonimidamide | 493 |
| 21 | 27B | 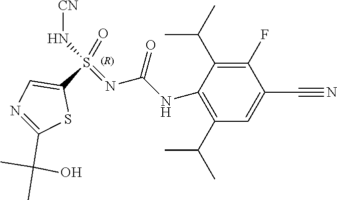 | (R) or (S)-N-cyano-N′-((4-cyano-3- fluoro-2,6- diisopropylphenyl)carbamoyl)-2- (2-hydroxypropan-2-yl)thiazole-5- sulfonimidamide | 493 |
| 22 | 28B | 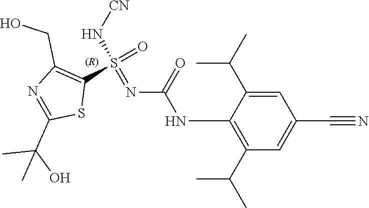 | (R) or (S)-N-cyano-N′-((4-cyano- 2,6-diisopropylphenyl)carbamoyl)- 4-(hydroxymethyl)-2-(2- hydroxypropan-2-yl)thiazole-5- sulfonimidamide | 505 |
| 23 | 29A | 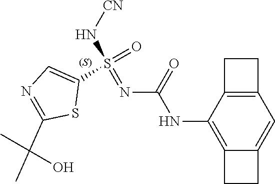 | (S) or (R)-N-cyano-2-(2- hydroxypropan-2-yl)-N′- (tricyclo[6.2.0.03,6]deca-1,3(6),7- trien-2-ylcarbamoyl)thiazole-5- sulfonimidamide | 418 |
| 24 | 29B | 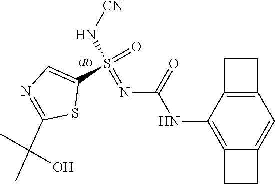 | (R) or (S)-N-cyano-2-(2- hydroxypropan-2-yl)-N′- (tricyclo[6.2.0.03,6]deca-1,3(6),7- trien-2-ylcarbamoyl)thiazole-5- sulfonimidamide | 418 |
| 25 | 30A | 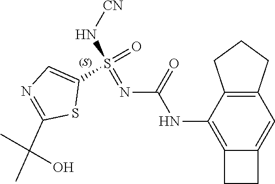 | (S) or (R)-N-cyano-2-(2- hydroxypropan-2-yl)-N′-((2,4,5,6- tetrahydro-1H-cyclobuta[f]inden- 3-yl)carbamoyl)thiazole-5- sulfonimidamide | 432 |
| 26 | 30B | 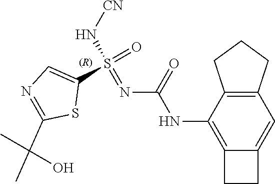 | (R) or (S)-N-cyano-2-(2- hydroxypropan-2-yl)-N′-((2,4,5,6- tetrahydro-1H-cyclobuta[f]inden-3- yl)carbamoyl)thiazole-5- sulfonimidamide | 432 |
| 27 | 31A | 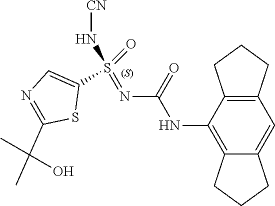 | (S) or (R)-N-cyano-N′- ((1,2,3,5,6,7-hexahydro-s-indacen- 4-yl)carbamoyl)-2-(2- hydroxypropan-2-yl)thiazole-5- sulfonimidamide | 446 |
| 28 | 31B | 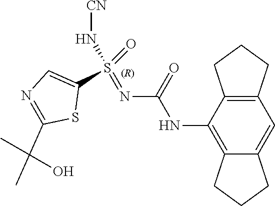 | (R) or (S)-N-cyano-N′- ((1,2,3,5,6,7-hexahydro-s-indacen- 4-yl)carbamoyl)-2-(2- hydroxypropan-2-yl)thiazole-5- sulfonimidamide | 446 |

Examples 29 to 33 may be prepared by analogous method to those described above, or alternatively according to the below syntheses.

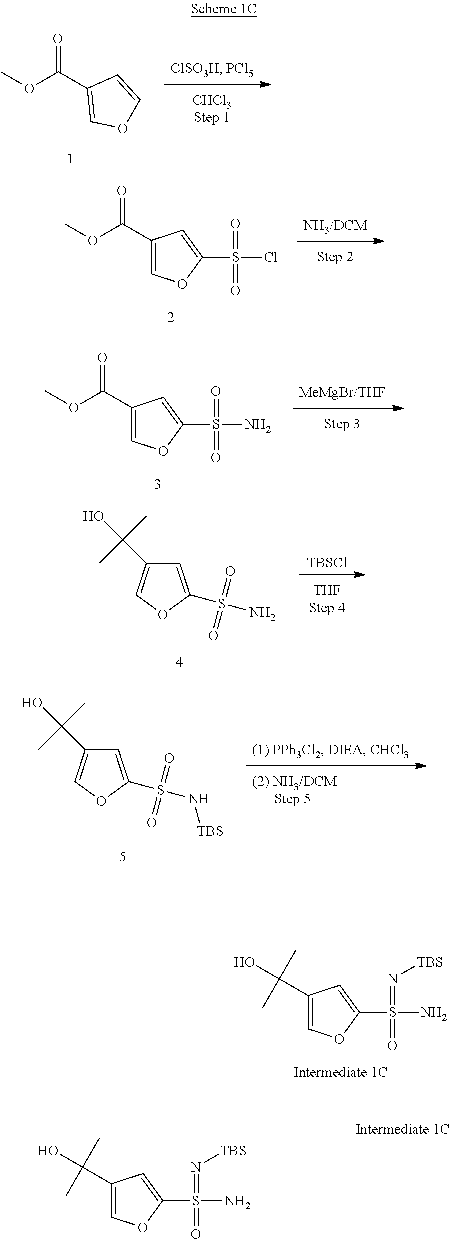

### N′-(tert-butyldimethylsilyl)-4-(2-hydroxypropan-2-yl)furan-2-sulfonimidamide

### Step 1: Methyl 5-(chlorosulfonyl)furan-3-carboxylate

To a stirred solution of methyl furan-3-carboxylate (6.3 g, 50 mmol) in CHCl₃ (200 mL) in a 500-mL 3-necked round-bottom flask was added chlorosulfonic acid (11.6 g, 100 mmol) dropwise at −10° C. Then the reaction mixture was stirred for 48 h at RT. Then to the above mixture was added phosphorus pentachloride (22.9 g, 110 mmol) in small poritions at −10° C. The resulting solution was stirred for 0.5 h at 50° C. The reaction was quenched by pouring into 200 mL of water/ice slowly. The resulting mixture was extracted with 3×200 mL of DCM. The organic layers were combined and dried over anhydrous Na₂SO₄, then concentrated under vacuum. This resulted in 7.0 g (crude) of the title compound as light brown oil. The crude product was used in the next step.

### Step 2: Methyl 5-sulfamoylfuran-3-carboxylate

To a stirred solution of methyl 5-(chlorosulfonyl)furan-3-carboxylate (7.0 g, crude) in DCM (75 mL) in a 250-mL round-bottom flask was bubbled NH₃(g) for 20 min at 0° C. in an ice/water bath. The resulting solution was stirred for 3 h at RT and concentrated under vacuum. The residue was eluted from silica gel with a gradient of EtOAc/PE (1:4 to 1:2). This resulted in 5.0 g (49% over two steps) of the title compound as a light yellow solid. MS-ESI: 204 (M−1).

### Step 3: 4-(2-Hydroxypropan-2-yl)furan-2-sulfonamide

To a stirred solution of methyl 5-sulfamoylfuran-3-carboxylate (3.46 g, 16.9 mmol) in THF (100 mL) in a 250-mL 3-necked round-bottom flask was added MeMgBr in THF (3 M, 25 mL) dropwise at −10° C. The resulting solution was stirred for 10 h at RT. Then the reaction solution was quenched with 50 mL of NH₄Cl (sat.). The resulting solution was extracted with 3×100 mL of EtOAc. The organic layers were combined and dried over anhydrous Na₂SO₄ and concentrated under vacuum. The residue was eluted from silica gel with a gradient of EtOAc/PE (1:3 to 1:1). This resulted in 2.6 g (75%) of the title compound as a light yellow solid. MS-ESI: 204 (M−1).

### Step 4: N-(tert-butyldimethylsilyl)-4-(2-hydroxypropan-2-yl)furan-2-sulfonamide

To a stirred solution of 4-(2-hydroxypropan-2-yl)furan-2-sulfonamide (2.6 g, 12.7 mmol) in THF (100 mL) in a 250-mL round-bottom flask under nitrogen was added NaH (60% wt. dispersion in mineral oil, 1.02 g, 25.5 mmol) in portions at 0° C. in an ice/water bath. The solution was stirred for 10 min at RT. To the stirred solution was added TBSCl (9.53 g, 63.5 mmol) in portions at 0° C. The resulting solution was stirred for 14 h at RT and then was diluted with 100 mL of water. The resulting mixture was extracted with 3×50 mL of DCM and the organic layers were combined and concentrated under vacuum. The residue was eluted from silica gel with a gradient of EtOAc/PE (1:10 to 1:3). This resulted in 3.73 g (92%) of the title compound as a white solid. MS-ESI: 318 (M−1).

### Step 5: N′-(tert-butyldimethylsilyl)-4-(2-hydroxypropan-2-yl)furan-2-sulfonimidamide

To a stirred solution of PPh₃Cl₂ (3.0 g, 10.2 mmol) in CHCl₃ (100 mL) in a 250-mL 3-necked round-bottom flask under nitrogen was added DIEA (2.63 g, 20.4 mmol) dropwise at 0° C. After stirred at 0° C. for 10 min, to the above mixture was added a solution of N-(tert-butyldimethylsilyl)-4-(2-hydroxypropan-2-yl) furan-2-sulfonamide (2.16 g, 6.8 mmol) in CHCl₃ (10 mL) dropwise with stirring at 0° C. The resulting solution was allowed to react for 30 min at RT. To the mixture was bubbled NH₃ (g) for 20 min at 0° C. The resulting solution was stirred for 2 h at RT. The reaction was then quenched by the addition of 100 mL of water. The resulting solution was extracted with 3×50 mL of DCM and the organic layers were combined and concentrated under vacuum. The residue was eluted from silica gel with a gradient of EtOAc/PE (1:10 to 1:3). This resulted in 1.15 g (53%) of the title compound as a light yellow solid. MS-ESI: 319 (M+1).

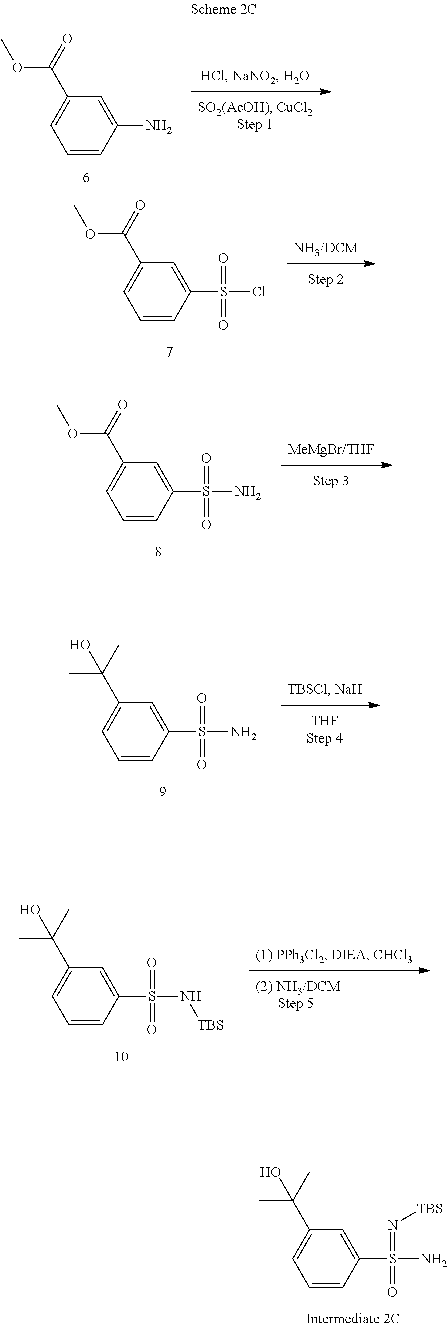

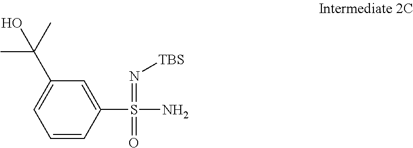

### N′-(tert-butyldimethylsilyl)-3-(2-hydroxypropan-2-yl)benzenesulfonimidamide

### Step 1: Methyl 3-(chlorosulfonyl)benzoate

To a stirred solution of methyl 3-aminobenzoate (2.76 g, 18.3 mmol) in HCl (6 M, 12 mL) in a 50-mL round-bottom flask was added NaNO₂ (1.5 g, 21.7 mmol) in water (3 mL) dropwise with stirring at 0° C. over 10 min. The resulting solution was stirred for 30 min at 0° C., this solution was assigned as solution A. Then to a stirred solution of CuCl₂ (4.8 g, 35.7 mmol) in AcOH (100 mL) in a 500-mL single necked round-bottom flask was bubbled SO₂ (g) at RT for 20 min, this solution was assigned as solution B. To the solution B was added solution A dropwise with stirring at 0° C. The resulting solution was stirred for 2 h at RT. The residue was diluted with 200 mL of water and extracted with 3×200 mL of DCM. The organic layers were combined and dried over anhydrous Na₂SO₄ and concentrated under vacuum. This resulted in 4.3 g (crude) of the title compound as yellow oil. The crude product was used in the next step.

### Step 2: Methyl 3-sulfamoylbenzoate

To a stirred solution of methyl 3-(chlorosulfonyl)benzoate (4.3 g, crude from last step) in DCM (20 mL) in a 250-mL round-bottom flask was bubbled NH₃(g) for 20 min at 0° C. The resulting solution was stirred for 2 h at RT and then was concentrated under vacuum. The resulting mixture was added 100 mL H₂O and extracted with 3×100 mL of EtOAc. The organic layers were dried over anhydrous Na₂SO₄. The solids were filtered out. The resulting filtrate was concentrated under vacuum. The residue was eluted from silica gel with a gradient of EtOAc/PE (1:5 to 1:3). This resulted in 2.79 g (71% over two steps) of the title compound as a white solid. MS-ESI: 214 (M−1).

Steps 3-5 used similar procedures for converting compound 3 to intermediate 1C shown in Scheme 1C to afford intermediate 2C from compound 8 of scheme 2C. MS-ESI: 329 (M+1).

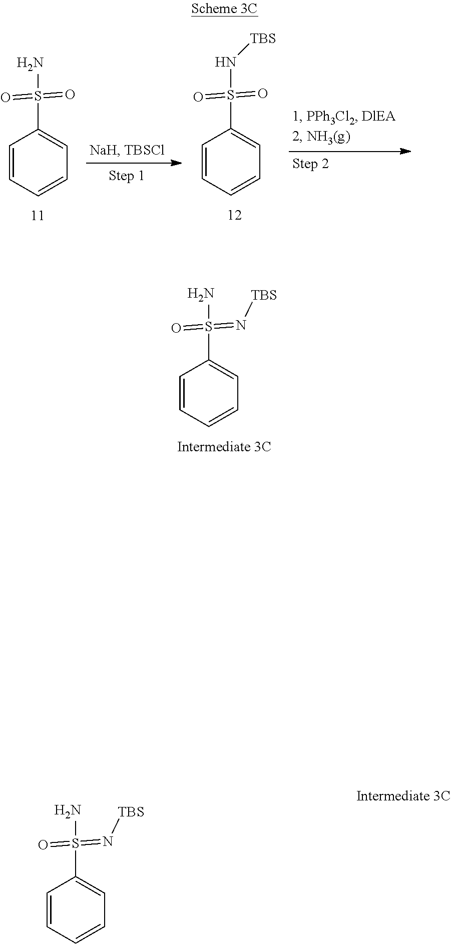

### N′-(tert-butyldimethylsilyl)benzenesulfonimidamide

Steps 1-2 used similar procedures for converting compound 4 of scheme 1C to intermediate 1C shown in Scheme 1C to afford intermediate 3C from compound 11 of scheme 3C. MS-ESI: 271 (M+1).

Intermediate 12

The synthesis of intermediate 12 of scheme 4C has been previously descried herein (schemes 7 and 10, above).

Schemes for phenylacetic acids Intermediates: Schemes 4C illustrate the coupling of LHS with RHS intermediates.

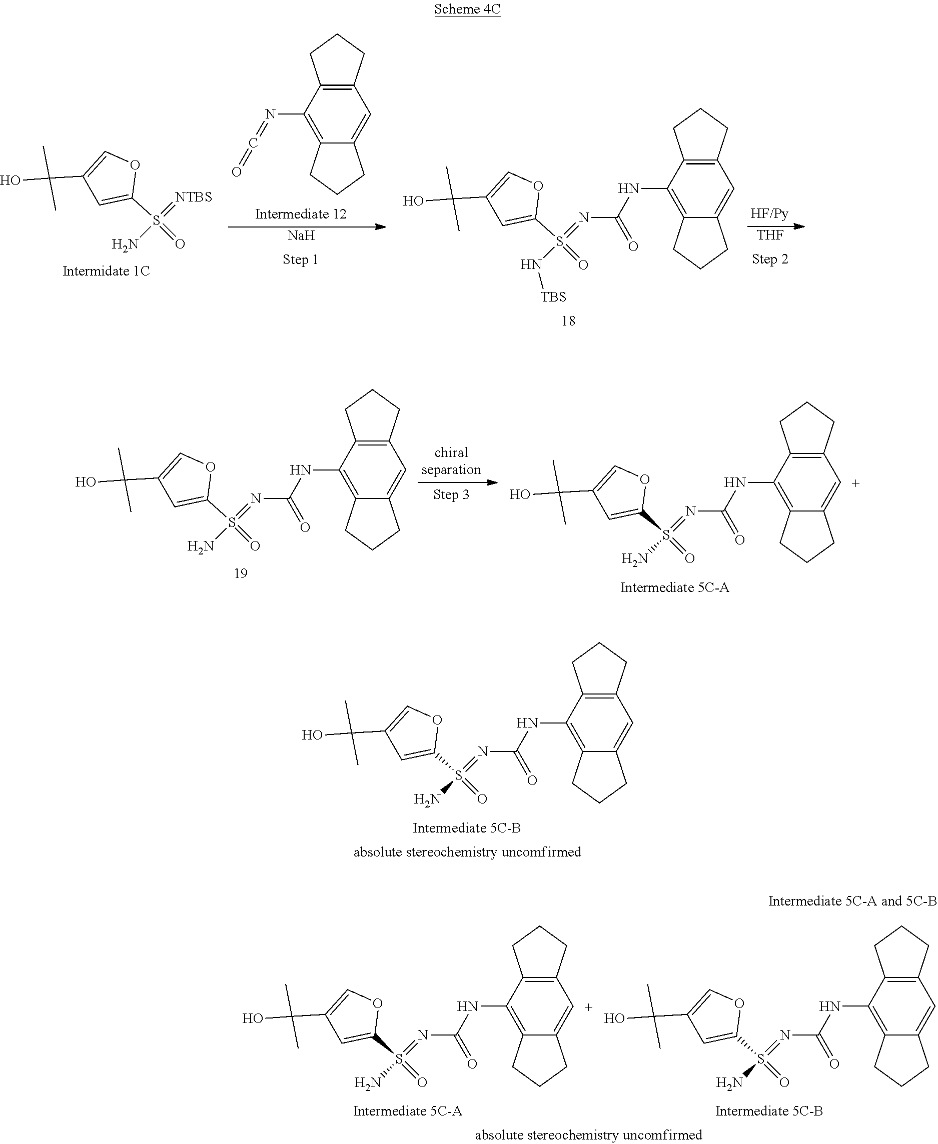

### (S) and (R)-N′-((1,2,3,5,6,7-hexahydro-s-indacen-4-yl)carbamoyl)-4-(2-hydroxypropan-2-yl)furan-2-sulfonimidamide

### Step 1: N-(tert-butyldimethylsilyl)-N′-((1,2,3,5,6,7-hexahydro-s-indacen-4-yl)carbamoyl)-4-(2-hydroxypropan-2-yl)furan-2-sulfonimidamide

To a stirred solution of N′-(tert-butyldimethylsilyl)-4-(2-hydroxypropan-2-yl)furan-2-sulfonimidamide (318 mg, 1.0 mmol) in THF (10 mL) in a 50-mL round-bottom flask under nitrogen was added NaH (60% wt. dispersion in mineral oil, 80 mg, 2.0 mmol) in portions at 0° C. in an ice/water bath. The resulting solution was stirred for 20 min at RT. Then 4-isocyanato-1,2,3,5,6,7-hexahydro-s-indacene (199 mg, 1.0 mmol) in THF (1.0 mL) was added dropwise at 0° C. The reaction solution was stirred for 2 h at RT. Then the reaction solution was quenched by the addition of 5.0 mL of MeOH. The resulting mixture was concentrated. The residue was eluted from silica gel with DCM/MeOH (20:1). This resulted in 300 mg (58%) of the title compound as a yellow solid. MS-ESI: 518 (M+1).

### Step 2: N-(tert-butyldimethylsilyl)-4-(2-hydroxypropan-2-yl)-N′-((2,4,5,6-tetrahydro-1H-cyclobuta[f]inden-3-yl)carbamoyl)thiophene-2-sulfonimidamide

To a stirred solution of N-(tert-butyldimethylsilyl)-N′-((1,2,3,5,6,7-hexahydro-s-indacen-4-yl)carbamoyl)-4-(2-hydroxypropan-2-yl)furan-2-sulfonimidamide (300 mg, 0.58 mmol) in THF (10 mL) in a 50-mL round-bottom flask was added HF/Pydine (70% wt., 33 mg, 1.16 mmol) dropwise at RT. The resulting solution was stirred for 2 h at RT. The resulting solution was concentrated under vacuum. The crude product was purified by Prep-HPLC with the following conditions: XBridge Prep OBD C18 Column, 19*250 mm, 5 um; mobile phase, Water(10 mM NH₄HCO₃) and MeCN (25% PhaseB up to 30% over 7 min); Detector, UV254/220 nm. This resulted in 190 mg (82%) of the title compound as a white solid. MS-ESI: 404 (M+1).

### Step 3: (R) and (S)-N′-((1,2,3,5,6,7-hexahydro-s-indacen-4-yl)carbamoyl)-4-(2-hydroxypropan-2-yl) furan-2-sulfonimidamide

The product of N′-((1,2,3,5,6,7-hexahydro-s-indacen-4-yl)carbamoyl)-4-(2-hydroxypropan-2-yl)furan-2- sulfonimidamide (100 mg) was purified by chiral separation with the following conditions: Column: CHIRAL ART Cellulose-SB, 2*25 cm, 5 um; mobile phase, Hex (0.2% DEA)(80%) and EtOH (40%); Detector: UV 210/254 nm. This resulted in 36.5 mg (36.5%, 99% ee) of Intermediate 5C-A followed by 38.6 mg (38.6%, 99%ee) of Intermediate 5C-B both as a white solid. MS-ESI: 404 (M+1).

TABLE 1C
The Intermediates in the following table were prepared using similar procedures for
converting Intermediate 1C to Intermediate 5C-A and 5C-B shown in Scheme 4C using
appropriated intermediates. The chiral column and eluents are listed in the table. As a convention,
the faster-eluting enantiomer is always listed first in the table followed by the slower-eluting
enantiomer of the pair. The symbol * at a chiral center denotes that this chiral center has been
resolved and the absolute stereochemistry at that center has not been determined. Assigned
stereochemistry in compound names are tentative.

| Num- | | | | | LC-MS |
|---|---|---|---|---|---|
| ber | Structure | IUPAC Name | Column | Eluents | [M + H]⁺ |
| Inter- mediate 6C-A | 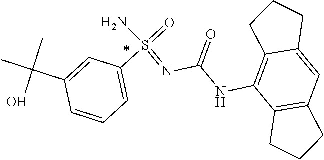 [SMILES: CC(C)(O)c1cccc(S(N)(=O)=NC(=O)Nc2c3c(cc4c2CCC4)CCC3)c1] | (S) or (R)-N′-((1,2,3,5,6,7- hexahydro-s-indacen-4- yl)carbamoyl)-3-(2- hydroxypropan-2-yl) benzenesulfonimidamide | ChiralPak IF, 0.46*5 cm; 3 um | 30% EtOH in Hex (0.1% DEA) | 414 |
| Inter- mediate 6C-B | 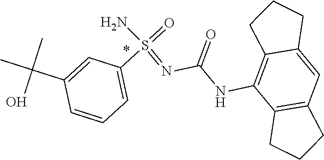 [SMILES: CC(C)(O)c1cccc(S(N)(=O)=NC(=O)Nc2c3c(cc4c2CCC4)CCC3)c1] | (R) or (S)-N′-((1,2,3,5,6,7- hexahydro-s-indacen-4- yl)carbamoyl)-3-(2- hydroxypropan-2-yl) benzenesulfonimidamide | ChiralPak IF, 0.46*5 cm; 3 um | 30% EtOH in Hex (0.1% DEA) | 414 |
| Inter- mediate 7C-A | 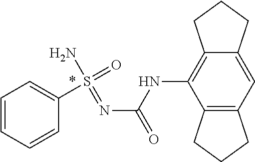 [SMILES: NS(=O)(=NC(=O)Nc1c2c(cc3c1CCC3)CCC2)c1ccccc1] | (S) or (R)-N′-((1,2,3,5,6,7- hexahydro-s-indacen-4- yl)carbamoyl) benzenesulfonimidamide | ChiralPak IA, 2*25 cm; 5 um | 30% EtOH in Hex (8 mM NH₃•MeOH) | 356 |
| Inter- mediate 7C-B | 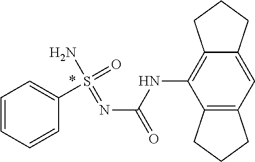 [SMILES: NS(=O)(=NC(=O)Nc1c2c(cc3c1CCC3)CCC2)c1ccccc1] | (R) or (S)-N′-((1,2,3,5,6,7- hexahydro-s-indacen-4- yl)carbamoyl) benzenesulfonimidamide | ChiralPak IA, 2*25 cm; 5 um | 30% EtOH in Hex (8 mM NH₃•MeOH) | 356 |

### Example 29

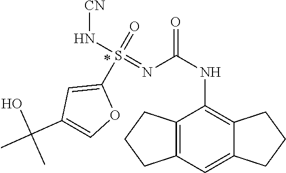 [SMILES: CC(C)(O)c1coc(S(=O)(=NC(=O)Nc2c3c(cc4c2CCC4)CCC3)NC#N)c1]

### (R) or (S)-N-cyano-N′-(1,2,3,5,6,7-hexahydro-s-indacen-4-ylcarbamoyl)-4-(2-hydroxypropan-2-yl)furan-2-sulfonimidamide (Scheme IC)

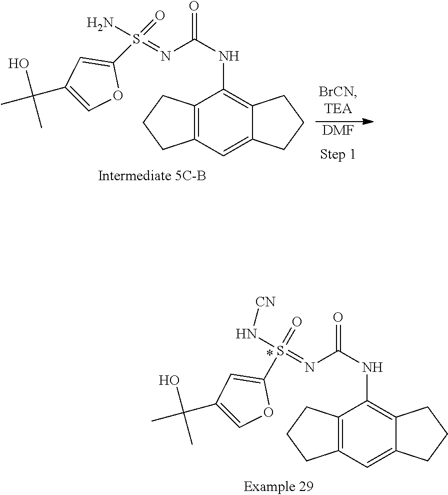

To a stirred solution of (R) or (S)-N′-(1,2,3,5,6,7-hexahydro-s-indacen-4-ylcarbamoyl)-4-(2-hydroxypropan-2-yl)furan-2-sulfonimidamide (3.7 mg, 0.0090 mmol) in DMF (0.5 mL) in a 8-mL vial was added TEA (5.0 mg, 0.049 mmol) at RT, followed by the addition of BrCN (2.0 mg, 0.019 mmol) in portions at RT. The resulting solution was stirred for 2 h at RT. The pH value of the solution was adjusted to 10 with NaOH (1 M). The resulting mixture was concentrated under vacuum. The crude product was purified by Prep-HPLC with the following conditions: XBridge Prep OBD C18 Column, 30×150 mm Sum; Mobile Phase A: Water (10 mM NH₄HCO₃+0.1% NH₃.H₂O), Mobile Phase B: MeCN; Flow rate: 60 mL/min; Gradient: 16% B to 46% B over 7 min; Detector, UV254/220 nm; Rt: 6.22 min; This resulted in 2.5 mg (64%) of Example 29 as a white solid. MS-ESI: 429 (M+1). ¹H NMR (400 MHz, DMSO-d₆) δ 8.04 (br s, 1H), 7.55 (s, 1H), 7.01 (br s, 1H), 6.82 (s, 1H), 6.79 (s, 1H), 5.01 (s, 1H), 2.80-2.71 (m, 4H), 2.70-2.65 (m, 4H), 1.94-1.89 (m, 4H), 1.38 (s, 6H).

TABLE 2C
Examples in the following table were prepared using similar conditions as described
in Example 29 and Scheme IC using appropriate intermediates.

| Ex. | From | | | Exact Mass |
|---|---|---|---|---|
| # | Int. # | Structure | IUPAC Name | [M + H]⁺ |
| 30 | 5C-A | 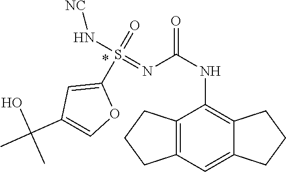 [SMILES: CC(C)(O)c1coc(S(=O)(=NC(=O)Nc2c3c(cc4c2CCC4)CCC3)NC#N)c1] | (S) or (R)-N-cyano-N′- ((1,2,3,5,6,7-hexahydro-s- indacen-4-yl)carbamoyl)-4-(2- hydroxypropan-2-yl)furan-2- sulfonimidamide | 429 |
| 31 | 6C-A | 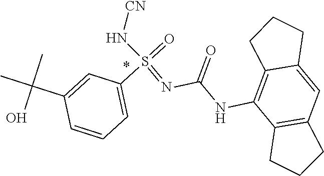 [SMILES: CC(C)(O)c1cccc(S(=O)(=NC(=O)Nc2c3c(cc4c2CCC4)CCC3)NC#N)c1] | (S) or (R)-N-cyano-N′- ((1,2,3,5,6,7-hexahydro-s- indacen-4-yl)carbamoyl)- 3-(2-hydroxypropan-2- yl)benzenesulfonimidamide | 439 |
| 32 | 6C-B | 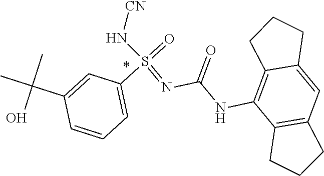 [SMILES: CC(C)(O)c1cccc(S(=O)(=NC(=O)Nc2c3c(cc4c2CCC4)CCC3)NC#N)c1] | (R) or (S)-N-cyano-N′- ((1,2,3,5,6,7-hexahydro-s- indacen-4-yl)carbamoyl)- 3-(2-hydroxypropan-2- yl)benzenesulfonimidamide | 439 |
| 33 | 7C-A | 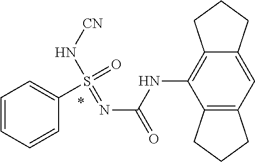 [SMILES: N#CNS(=O)(=NC(=O)Nc1c2c(cc3c1CCC3)CCC2)c1ccccc1] | (S) or (R)-N-cyano-N′- ((1,2,3,5,6,7-hexahydro-s- indacen-4-yl)carbamoyl) benzenesulfonimidamide | 381 |
| 34 | 7C-B | 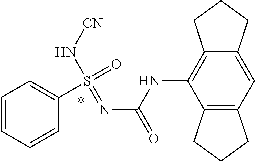 [SMILES: N#CNS(=O)(=NC(=O)Nc1c2c(cc3c1CCC3)CCC2)c1ccccc1] | (R) or (S)-N-cyano-N′- ((1,2,3,5,6,7-hexahydro-s- indacen-4-yl)carbamoyl) benzenesulfonimidamide | 381 |

The following examples can be prepared according to the methods described above.

(R)-N′-cyano-N-((1,2,3,5,6,7-hexahydro-s-indacen-4-yl)carbamoyl)-4-methylbenzene sulfonimidamide;

(R)-N′-cyano-N-((1,2,3,5,6,7-hexahydro-s-indacen-4-yl)carbamoyl)-4-methylbenzene sulfonimidamide;

(R)-N′-cyano-N-((2,6-diisopropylphenyl)carbamoyl)-4-methylbenzenesulfonimidamide;

(R)-N-((2,6-diisopropylphenyl)carbamoyl)-4-methyl-N′-(2,2,2-trifluoroethyl)benzenesulfonimi-damide;

(R)-N-((1,2,3,5,6,7-hexahydro-s-indacen-4-yl)carbamoyl)-4-methyl-N′-(2,2,2-trifluoroethyl)-benzenesulfonimidamide;

(R)-N-((2,6-diisopropylphenyl)carbamoyl)-N′-methoxy-4-methylbenzenesulfonimidamide;

(R)-N-((1,2,3,5,6,7-hexahydro-s-indacen-4-yl)carbamoyl)-N′-isopropyl-4-methylbenzenesulfonimidamide;

(R)-N-((2,6-diisopropylphenyl)carbamoyl)-N′-isopropyl-4-methylbenzenesulfonimidamide;

(R)-N-((1,2,3,5,6,7-hexahydro-s-indacen-4-yl)carbamoyl)-N′-methoxy-4-methylbenzenesulfo-nimidamide;

(R)-N-((1,2,3,5,6,7-hexahydro-s-indacen-4-yl)carbamoyl)-N′,4-dimethylbenzenesulfonimidamide;

(R)-N-((1,2,3,5,6,7-hexahydro-s-indacen-4-yl)carbamoyl)-4-methyl-N′-(1H-pyrazol-5-yl)benzenesulfonimidamide;

(R)-N′-(4-fluorophenyl)-N-((1,2,3,5,6,7-hexahydro-s-indacen-4-yl)carbamoyl)-4-methylbenzenesulfonimidamide;

(R)-N-((1,2,3,5,6,7-hexahydro-s-indacen-4-yl)carbamoyl)-4-methyl-N′-(pyridin-2-yl)benzenesulfonimidamide;

(R)-4-acetyl-N′-cyano-N-((1,2,3,5,6,7-hexahydro-s-indacen-4-yl)carbamoyl)benzenesulfo-nimidamide;

(R)-N′-cyano-N-((1,2,3,5,6,7-hexahydro-s-indacen-4-yl)carbamoyl)-4-(2-hydroxypropan-2-yl)benzenesulfonimidamide;

(R)-N′-cyano-N-((1,2,3,5,6,7-hexahydro-s-indacen-4-yl)carbamoyl)-3-nitrobenzenesulfo-nimidamide;

(R)-N′-cyano-N-((1,2,3,5,6,7-hexaydro-s-indacen-4-yl)carbamoyl)-4-methoxybenzenesulfonimidamide;

(R)-N′-cyano-N-((2,6-diisopropylphenyl)carbamoyl)-4-methoxybenzenesulfonimidamide;

(R)-N′-cyano-4-fluoro-N-((1,2,3,5,6,7-hexahydro-s-indacen-4-yl)carbamoyl)benzenesulfo-nimidamide;

(R)-N′-cyano-N-((1,2,3,5,6,7-hexahydro-s-indacen-4-yl)carbamoyl)benzenesulfonimidamide;

(R)-N-((3-(1,2,3,5,6,7-hexahydro-s-indacen-4-yl)ureido)(oxo)(p-tolyl)-{lambda}6-sulfanylidene)-methanesulfonamide;

(R)-N′-cyano-N-((1,2,3,5,6,7-hexahydro-s-indacen-4-yl)carbamoyl)-6-methoxypyridine-3-sulfonimidamide;

(R)-N′-cyano-3-fluoro-N-((1,2,3,5,6,7-hexahydro-s-indacen-4-yl)carbamoyl)-4-methyl-benzenesulfonimidamide;

(R)-4-chloro-N′-cyano-N-((1,2,3,5,6,7-hexahydro-s-indacen-4-yl)carbamoyl)benzene-sulfonimidamide;

(R)-4-bromo-N′-cyano-N-((1,2,3,5,6,7-hexahydro-s-indacen-4-yl)carbamoyl)benzene-sulfonimidamide;

(R)-4-(benzyloxy)-N′-cyano-N-((1,2,3,5,6,7-hexahydro-s-indacen-4-yl)carbamoyl)benzene-sulfonimidamide;

(R)-N′-cyano-N-((1,2,3,5,6,7-hexahydro-s-indacen-4-yl)carbamoyl)-4-(trifluoromethyl)-benzenesulfonimidamide;

(R)-N′-cyano-N-((1,2,3,5,6,7-hexahydro-s-indacen-4-yl)carbamoyl)pyridine-2-sulfonimidamide;

(R)-Ethyl-((3-(1,2,3,5,6,7-hexahydro-s-indacen-4-yl)ureido)(oxo)(p-tolyl)-{lambda}6-sulfaneylidene)-carbamate;

(R)-N-((3-(1,2,3,5,6,7-hexahydro-s-indacen-4-yl)ureido)(oxo)-(p-tolyl)-{lambda}6-sulfaneylidene)-acetamide;

(R)-N′-carbamoyl-N-((1,2,3,5,6,7-hexahydro-s-indacen-4-yl)carbamoyl)-4-methylbenzene-sulfonimidamide;

(R)-N′-cyano-N-((1,2,3,5,6,7-hexahydro-s-indacen-4-yl)carbamoyl)-3-(2-hydroxypropan-2-yl)benzenesulfonimidamide;

(R)-N′-cyano-N-((1,2,3,5,6,7-hexahydro-s-indacen-4-yl)carbamoyl)-4-(3-hydroxyoxetan-3-yl)benzenesulfonimidamide;

(R)-N′-cyano-N-((1,2,3,5,6,7-hexahydro-s-indacen-4-yl)carbamoyl)-4-(prop-1-en-2-yl)furan-2-sulfonimidamide;

(R)-5-bromo-N′-cyano-N-((1,2,3,5,6,7-hexahydro-s-indacen-4-yl)carbamoyl)thiophene-2-sulfonimidamide;

(R)-N′-cyano-N-((1,2,3,5,6,7-hexahydro-s-indacen-4-yl)carbamoyl)-3-methoxybenzene-sulfonimidamide;

(R)-N′-cyano-N-((1,2,3,5,6,7-hexahydro-s-indacen-4-yl)carbamoyl)-4-(2-hydroxypropan-2-yl)furan-2-sulfonimidamide;

(R)-N′-cyano-N-((1,2,3,5,6,7-hexahydro-s-indacen-4-yl)carbamoyl)-2,3-dihydrobenzo[b][1,4]dioxine-6-sulfonimidamide;

(R)-N′-cyano-N-((1,2,3,5,6,7-hexahydro-s-indacen-4-yl)carbamoyl)-5-(2-hydroxypropan-2-yl)thiophene-2-sulfonimidamide;

(R)-N′-cyano-2-fluoro-N-((1,2,3,5,6,7-hexahydro-s-indacen-4-yl)carbamoyl)-5-methylbenzene-sulfonimidamide;

(R)-N′-cyano-3-(1,1,1,3,3,3-hexafluoro-2-hydroxypropan-2-yl)-N-((1,2,3,5,6,7-hexahydro-s-indacen-4-yl)carbamoyl)benzenesulfonimidamide;

(R)-N′-cyano-3,5-difluoro-N-((1,2,3,5,6,7-hexahydro-s-indacen-4-yl)carbamoyl)benzene-sulfonimidamide;

(R)-N′-cyano-2,4-difluoro-N-((1,2,3,5,6,7-hexahydro-s-indacen-4-yl)carbamoyl)benzene-sulfonimidamide;

(R)-N′-cyano-N-((1,2,3,5,6,7-hexahydro-s-indacen-4-yl)carbamoyl)-3-(trifluoromethoxy)-benzenesulfonimidamide;

(R)-N′-cyano-N-((2,6-diisopropylphenyl)carbamoyl)-3-(2-hydroxypropan-2-yl)benzene-sulfonimidamide;

(R)-N′-cyano-N-((1,2,3,5,6,7-hexahydro-s-indacen-4-yl)carbamoyl)-1-phenylmethanesulfonimidamide;

(R)-N-((3-(1,2,3,5,6,7-hexahydro-s-indacen-4-yl)ureido)(3-(2-hydroxypropan-2-yl)phenyl)(oxo)-{lambda}6-sulfaneylidene)methanesulfonamide;

(R)-N′-cyano-N-((1,2,3,6,7,8-hexahydro-as-indacen-4-yl)carbamoyl)-4-methylbenzenesulfo-nimidamide;

(R)-N′-cyano-N-((1,2,3,5,6,7-hexahydro-s-indacen-4-yl)carbamoyl)thiophene-2-sulfonimidamide;

(R)-N′-cyano-N-((1,2,3,5,6,7-hexahydro-s-indacen-4-yl)carbamoyl)-3,4-dimethoxybenzenesulfonimidamide;

(R)-N′-cyano-N-((1,2,3,5,6,7-hexahydro-s-indacen-4-yl)carbamoyl)-6-methylpyridine-2-sulfonimidamide;

(R)-N′-cyano-N-((1,2,3,5,6,7-hexahydro-s-indacen-4-yl)carbamoyl)-6-methylpyridine-2-sulfonimidamide;

(R)-N′,3-dicyano-N-((1,2,3,5,6,7-hexahydro-s-indacen-4-yl)carbamoyl)benzenesulfonimidamide;

(R)-N′-cyano-N-((1,2,3,5,6,7-hexahydro-s-indacen-4-yl)carbamoyl)pyridine-3-sulfonimidamide;

(R)-N′,4-dicyano-N-((1,2,3,5,6,7-hexahydro-s-indacen-4-yl)carbamoyl)benzene-sulfonimidamide;

(R)-N′-cyano-N-((1,2,3,5,6,7-hexahydro-s-indacen-4-yl)carbamoyl)pyridine-4-sulfonimidamide;

(R)-N′-cyano-3-fluoro-N-((1,2,3,5,6,7-hexahydro-s-indacen-4-yl)carbamoyl)benzene-sulfonimidamide;

(R)-N′-cyano-N-((1,2,3,5,6,7-hexahydro-s-indacen-4-yl)carbamoyl)quinoline-8-sulfonimidamide;

(R)-N′-cyano-N-((1,2,3,5,6,7-hexahydro-s-indacen-4-yl)carbamoyl)-4-morpholinobenzene sulfonimidamide;

(R)-N-(3-(N′-cyano-N-((1,2,3,5,6,7-hexahydro-s-indacen-4-yl)carbamoyl)sulfamidimidoyl)phenyl)acetamide;

(R)-N′-cyano-N-((1,2,3,5,6,7-hexahydro-s-indacen-4-yl)carbamoyl)-3-morpholinobenzene sulfonimidamide;

(R)-N′-cyano-4-fluoro-N-((1,2,3,5,6,7-hexahydro-s-indacen-4-yl)carbamoyl)-3-(2-hydroxypropan-2-yl)benzenesulfonimidamide;

(R)-N′-cyano-2-fluoro-N-((1,2,3,5,6,7-hexahydro-s-indacen-4-yl)carbamoyl)-5-(2-hydroxypropan-2-yl)benzenesulfonimidamide;

(R)-N′-cyano-N-((1,2,3,5,6,7-hexahydro-s-indacen-4-yl)carbamoyl)-5-(2-hydroxypropan-2-yl)pyridine-3-sulfonimidamide;

(R)-N′-cyano-1-(4-cyanophenyl)-N-((1,2,3,5,6,7-hexahydro-s-indacen-4-yl)carbamoyl)-methanesulfonimidamide;

(R)-N′-cyano-3-(2-hydroxypropan-2-yl)-N-((3-oxo-1,2,3,5,6,7-hexahydro-s-indacen-4-yl)carba-moyl)-benzenesulfonimidamide;

(S)-N′-cyano-N-((1,2,3,5,6,7-hexahydro-s-indacen-4-yl)carbamoyl)-4-methylbenzene sulfonimidamide;

(S)-N′-cyano-N-((1,2,3,5,6,7-hexahydro-s-indacen-4-yl)carbamoyl)-4-methylbenzene sulfonimidamide;

(S)-N′-cyano-N-((2,6-diisopropylphenyl)carbamoyl)-4-methylbenzenesulfonimidamide;

(S)-N-((2,6-diisopropylphenyl)carbamoyl)-4-methyl-N′-(2,2,2-trifluoroethyl)benzenesulfonimi-damide;

(S)-N-((1,2,3,5,6,7-hexahydro-s-indacen-4-yl)carbamoyl)-4-methyl-N′-(2,2,2-trifluoroethyl)-benzenesulfonimidamide;

(S)-N-((2,6-diisopropylphenyl)carbamoyl)-N′-methoxy-4-methylbenzenesulfonimidamide;

(S)-N-((1,2,3,5,6,7-hexahydro-s-indacen-4-yl)carbamoyl)-N′-isopropyl-4-methylbenzenesulfonimidamide;

(S)-N-((2,6-diisopropylphenyl)carbamoyl)-N′-isopropyl-4-methylbenzenesulfonimidamide;

(S)-N-((1,2,3,5,6,7-hexahydro-s-indacen-4-yl)carbamoyl)-N′-methoxy-4-methylbenzenesulfo-nimidamide;

(S)-N-((1,2,3,5,6,7-hexahydro-s-indacen-4-yl)carbamoyl)-N′,4-dimethylbenzenesulfonimidamide;

(S)-N-((1,2,3,5,6,7-hexahydro-s-indacen-4-yl)carbamoyl)-4-methyl-N′-(1H-pyrazol-5-yl)benzenesulfonimidamide;

(S)-N′-(4-fluorophenyl)-N-((1,2,3,5,6,7-hexahydro-s-indacen-4-yl)carbamoyl)-4-methylbenzenesulfonimidamide;

(S)-N-((1,2,3,5,6,7-hexahydro-s-indacen-4-yl)carbamoyl)-4-methyl-N′-(pyridin-2-yl)benzenesulfonimidamide;

(S)-4-acetyl-N′-cyano-N-((1,2,3,5,6,7-hexahydro-s-indacen-4-yl)carbamoyl)benzenesulfo-nimidamide;

(S)-N′-cyano-N-((1,2,3,5,6,7-hexahydro-s-indacen-4-yl)carbamoyl)-4-(2-hydroxypropan-2-yl)benzenesulfonimidamide;

(S)-N′-cyano-N-((1,2,3,5,6,7-hexahydro-s-indacen-4-yl)carbamoyl)-3-nitrobenzenesulfo-nimidamide;

(S)-N′-cyano-N-((1,2,3,5,6,7-hexaydro-s-indacen-4-yl)carbamoyl)-4-methoxybenzenesulfonimidamide;

(S)-N′-cyano-N-((2,6-diisopropylphenyl)carbamoyl)-4-methoxybenzenesulfonimidamide;

(S)-N′-cyano-4-fluoro-N-((1,2,3,5,6,7-hexahydro-s-indacen-4-yl)carbamoyl)benzenesulfo-nimidamide;

(S)-N′-cyano-N-((1,2,3,5,6,7-hexahydro-s-indacen-4-yl)carbamoyl)benzenesulfonimidamide;

(S)-N-((3-(1,2,3,5,6,7-hexahydro-s-indacen-4-yl)ureido)(oxo)(p-tolyl)-{lambda}6-sulfanylidene)-methanesulfonamide;

(S)-N′-cyano-N-((1,2,3,5,6,7-hexahydro-s-indacen-4-yl)carbamoyl)-6-methoxypyridine-3-sulfonimidamide;

(S)-N′-cyano-3-fluoro-N-((1,2,3,5,6,7-hexahydro-s-indacen-4-yl)carbamoyl)-4-methyl-benzenesulfonimidamide;

(S)-4-chloro-N′-cyano-N-((1,2,3,5,6,7-hexahydro-s-indacen-4-yl)carbamoyl)benzene-sulfonimidamide;

(S)-4-bromo-N′-cyano-N-((1,2,3,5,6,7-hexahydro-s-indacen-4-yl)carbamoyl)benzene-sulfonimidamide;

(S)-4-(benzyloxy)-N′-cyano-N-((1,2,3,5,6,7-hexahydro-s-indacen-4-yl)carbamoyl)benzene-sulfonimidamide;

(S)-N′-cyano-N-((1,2,3,5,6,7-hexahydro-s-indacen-4-yl)carbamoyl)-4-(trifluoromethyl)-benzenesulfonimidamide;

(S)-N′-cyano-N-((1,2,3,5,6,7-hexahydro-s-indacen-4-yl)carbamoyl)pyridine-2-sulfonimidamide;

(S)-Ethyl4(3-(1,2,3,5,6,7-hexahydro-s-indacen-4-yl)ureido)(oxo)(p-tolyl)-{lambda}6-sulfaneylidene)-carbamate;

(S)-N-((3-(1,2,3,5,6,7-hexahydro-s-indacen-4-yl)ureido)(oxo)-(p-tolyl)-{lambda}6-sulfaneylidene)-acetamide;

(S)-N′-carbamoyl-N-((1,2,3,5,6,7-hexahydro-s-indacen-4-yl)carbamoyl)-4-methylbenzene-sulfonimidamide;

(S)-N′-cyano-N-((1,2,3,5,6,7-hexahydro-s-indacen-4-yl)carbamoyl)-3-(2-hydroxypropan-2-yl)benzenesulfonimidamide;

(S)-N′-cyano-N-((1,2,3,5,6,7-hexahydro-s-indacen-4-yl)carbamoyl)-4-(3-hydroxyoxetan-3-yl)benzenesulfonimidamide;

(S)-N′-cyano-N-((1,2,3,5,6,7-hexahydro-s-indacen-4-yl)carbamoyl)-4-(prop-1-en-2-yl)furan-2-sulfonimidamide;

(S)-5-bromo-N′-cyano-N-((1,2,3,5,6,7-hexahydro-s-indacen-4-yl)carbamoyl)thiophene-2-sulfonimidamide;

(S)-N′-cyano-N-((1,2,3,5,6,7-hexahydro-s-indacen-4-yl)carbamoyl)-3-methoxybenzene-sulfonimidamide;

(S)-N′-cyano-N-((1,2,3,5,6,7-hexahydro-s-indacen-4-yl)carbamoyl)-4-(2-hydroxypropan-2-yl)furan-2-sulfonimidamide;

(S)-N′-cyano-N-((1,2,3,5,6,7-hexahydro-s-indacen-4-yl)carbamoyl)-2,3-dihydrobenzo[b][1,4]dioxine-6-sulfonimidamide;

(S)-N′-cyano-N-((1,2,3,5,6,7-hexahydro-s-indacen-4-yl)carbamoyl)-5-(2-hydroxypropan-2-yl)thiophene-2-sulfonimidamide;

(S)-N′-cyano-2-fluoro-N-((1,2,3,5,6,7-hexahydro-s-indacen-4-yl)carbamoyl)-5-methylbenzene-sulfonimidamide;

(S)-N′-cyano-3-(1,1,1,3,3,3-hexafluoro-2-hydroxypropan-2-yl)-N-((1,2,3,5,6,7-hexahydro-s-indacen-4-yl)carbamoyl)benzenesulfonimidamide;

(S)-N′-cyano-3,5-difluoro-N-((1,2,3,5,6,7-hexahydro-s-indacen-4-yl)carbamoyl)benzene-sulfonimidamide;

(S)-N′-cyano-2,4-difluoro-N-((1,2,3,5,6,7-hexahydro-s-indacen-4-yl)carbamoyl)benzene-sulfonimidamide;

(S)-N′-cyano-N-((1,2,3,5,6,7-hexahydro-s-indacen-4-yl)carbamoyl)-3-(trifluoromethoxy)-benzenesulfonimidamide;

(S)-N′-cyano-N-((2,6-diisopropylphenyl)carbamoyl)-3-(2-hydroxypropan-2-yl)benzene-sulfonimidamide;

(S)-N′-cyano-N-((1,2,3,5,6,7-hexahydro-s-indacen-4-yl)carbamoyl)-1-phenylmethanesulfonimidamide;

(S)-N-((3-(1,2,3,5,6,7-hexahydro-s-indacen-4-yl)ureido)(3-(2-hydroxypropan-2-yl)phenyl)(oxo)-{lambda}6-sulfaneylidene)methanesulfonamide;

(S)-N′-cyano-N-((1,2,3,6,7,8-hexahydro-as-indacen-4-yl)carbamoyl)-4-methylbenzenesulfo-nimidamide;

(S)-N′-cyano-N-((1,2,3,5,6,7-hexahydro-s-indacen-4-yl)carbamoyl)thiophene-2-sulfonimidamide;

(S)-N′-cyano-N-((1,2,3,5,6,7-hexahydro-s-indacen-4-yl)carbamoyl)-3,4-dimethoxybenzenesulfonimidamide;

(S)-N′-cyano-N-((1,2,3,5,6,7-hexahydro-s-indacen-4-yl)carbamoyl)-6-methylpyridine-2-sulfonimidamide;

(S)-N′-cyano-N-((1,2,3,5,6,7-hexahydro-s-indacen-4-yl)carbamoyl)-6-methylpyridine-2-sulfonimidamide;

(S)-N′,3-dicyano-N-((1,2,3,5,6,7-hexahydro-s-indacen-4-yl)carbamoyl)benzenesulfonimidamide;

(S)-N′-cyano-N-((1,2,3,5,6,7-hexahydro-s-indacen-4-yl)carbamoyl)pyridine-3-sulfonimidamide;

(S)-N′,4-dicyano-N-((1,2,3,5,6,7-hexahydro-s-indacen-4-yl)carbamoyl)benzene-sulfonimidamide;

(S)-N′-cyano-N-((1,2,3,5,6,7-hexahydro-s-indacen-4-yl)carbamoyl)pyridine-4-sulfonimidamide;

(S)-N′-cyano-3-fluoro-N-((1,2,3,5,6,7-hexahydro-s-indacen-4-yl)carbamoyl)benzene-sulfonimidamide;

(S)-N′-cyano-N-((1,2,3,5,6,7-hexahydro-s-indacen-4-yl)carbamoyl)quinoline-8-sulfonimidamide;

(S)-N′-cyano-N-((1,2,3,5,6,7-hexahydro-s-indacen-4-yl)carbamoyl)-4-morpholinobenzene sulfonimidamide;

(S)-N-(3-(N′-cyano-N-((1,2,3,5,6,7-hexahydro-s-indacen-4-yl)carbamoyl)sulfamidimidoyl)phenyl)acetamide;

(S)-N′-cyano-N-((1,2,3,5,6,7-hexahydro-s-indacen-4-yl)carbamoyl)-3-morpholinobenzene sulfonimidamide;

(S)-N′-cyano-4-fluoro-N-((1,2,3,5,6,7-hexahydro-s-indacen-4-yl)carbamoyl)-3-(2-hydroxypropan-2-yl)benzenesulfonimidamide;

(S)-N′-cyano-2-fluoro-N-((1,2,3,5,6,7-hexahydro-s-indacen-4-yl)carbamoyl)-5-(2-hydroxypropan-2-yl)benzenesulfonimidamide;

(S)-N′-cyano-N-((1,2,3,5,6,7-hexahydro-s-indacen-4-yl)carbamoyl)-5-(2-hydroxypropan-2-yl)pyridine-3-sulfonimidamide;

(S)-N′-cyano-1-(4-cyanophenyl)-N-((1,2,3,5,6,7-hexahydro-s-indacen-4-yl)carbamoyl)-methanesulfonimidamide; and

(S)-N′-cyano-3-(2-hydroxypropan-2-yl)-N-((3-oxo-1,2,3,5,6,7-hexahydro-s-indacen-4-yl)carba-moyl)-benzenesulfonimidamide.

Examples 34 to may be prepared by analogous method to those described above, or alternatively according to the below syntheses.

Schemes of Sulfonimidamide and amino pyridines Intermediates: Schemes below illustrate the preparation of sulfonimidamide and amino pyridines intermediates.

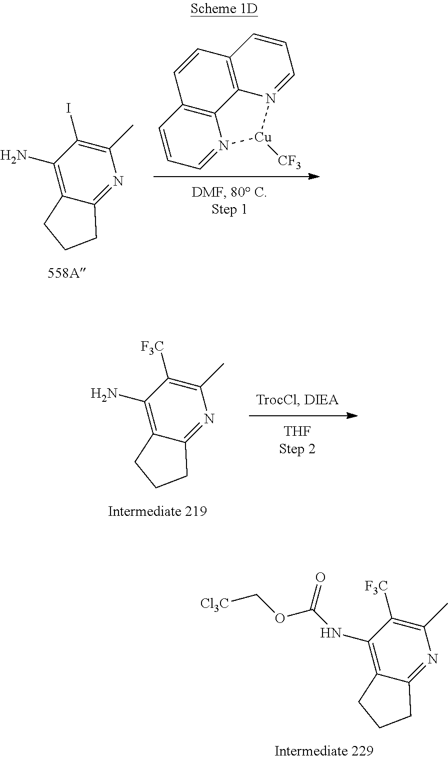

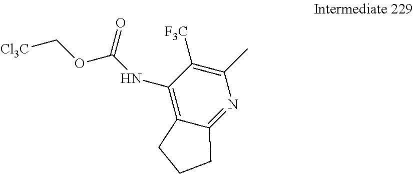

### 2,2,2-Trichloroethyl (2-methyl-3-(trifluoromethyl)-6,7-dihydro-5H-cyclopenta[b]pyridin-4-yl)carbamate

Step 1 used identical procedure for converting compound 558A″ to Intermediate 219 shown in Scheme 129 but used different purification method: The crude product was purified by reverse column using the following condition: Column: C18 spherical 20-35 um, 100 A, 330 g; Mobile Phase A: water (10 mM NH₄HCO₃), Mobile Phase B: ACN; Flow rate: 60 mL/min; Gradient: 10% B to 80% B over 90 min; 254/210 nm; Rt: 70 min. This resulted in 260 mg (impure, containing [Cu]) of Intermediate 219 as a dark-green solid. MS-ESI: 217 (M+1)

### Step 2: 2,2,2-trichloroethyl (2-methyl-3-(trifluoromethyl)-6,7-dihydro-5H-cyclopentaiblpyridin-4-yl)carbamate

To a stirred solution of 2-methyl-3-(trifluoromethyl)-6,7-dihydro-5H-cyclopenta[b]pyridin-4-amine (100 mg, 0.463 mmol) in THF (5 mL) was added DIEA (299 mg, 2.31 mmol) dropwise at 0° C. The resulting solution was stirred for 20 min at RT. This was followed by the addition of 2,2,2-trichloroethyl carbonochloridate (1.76 g, 8.33 mmol) dropwise with stirring at 0° C. The resulting solution was stirred for 16 h at 45° C. The reaction was quenched with 20 mL of water and extracted with 3×20 mL of EtOAc. The organic layers were combined and dried over anhydrous Na₂SO₄ and concentrated under vacuum. The crude product was purified by Prep-TLC with EtOAc/PE (1:9). This resulted in 74 mg (41%) of the title compound as white solid. MS-ESI: 391/393/395 (M+1)

TABLE 73
The Intermediate 265 in the following Table were prepared using similar procedures
for converting compound 558A″ to Intermediate 229 shown in Scheme 1D from appropriate
reagents.

| Intermediate # | Structure | IUPAC Name | Exact Mass [M + H]⁺ |
|---|---|---|---|
| Intermediate 265 | 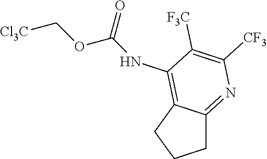 | 2,2,2-Trichloroethyl (2,3- bis(trifluoromethyl)-6,7-dihydro- 5H-cyclopenta[b]pyridin-4- yl)carbamate | 445/447/449 |

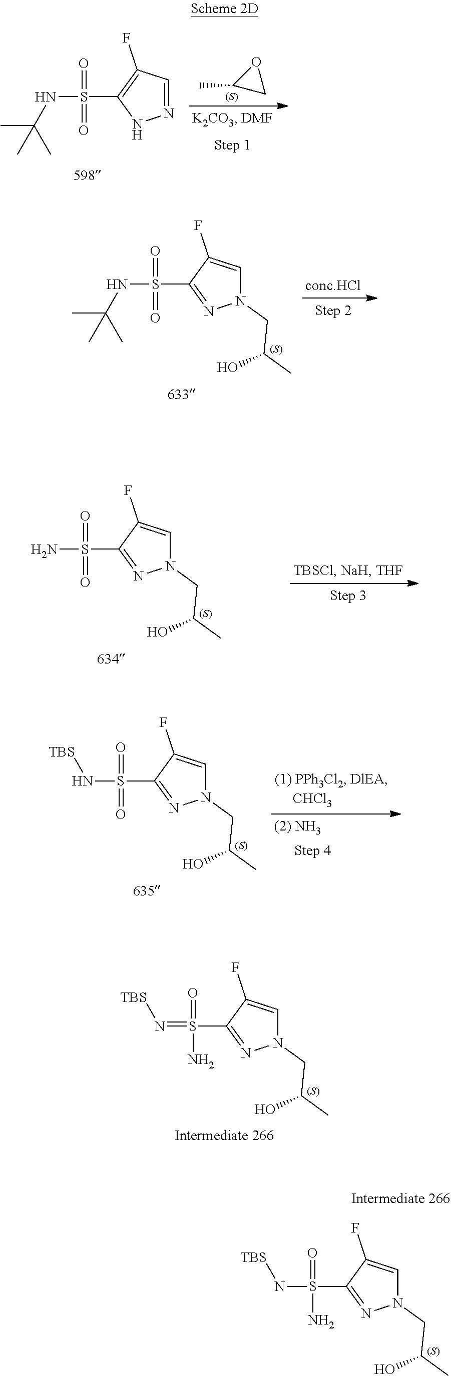

### N′-(tert-butyldimethylsilyl)-4-fluoro-1-((S)-2-hydroxypropyl)-1H-pyrazole-3-sulfonimidamide

Steps 1-4 used similar procedures for converting compound 598″ to Intermediate 262 shown in Scheme 2D to afford Intermediate 266 from compound 598″. MS-ESI: 337 (M+1).

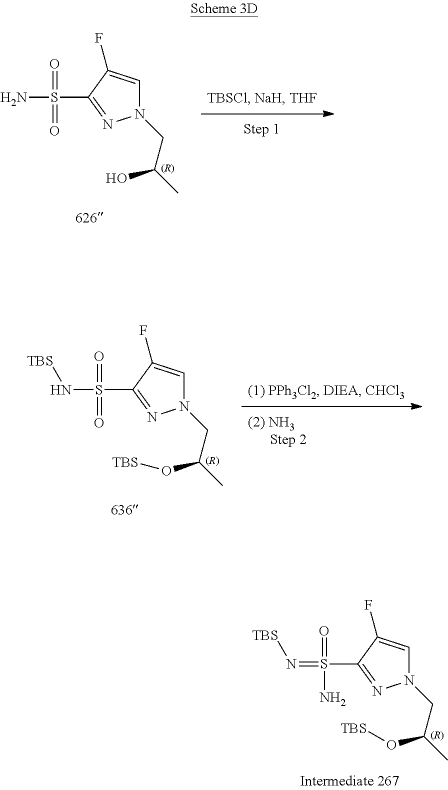

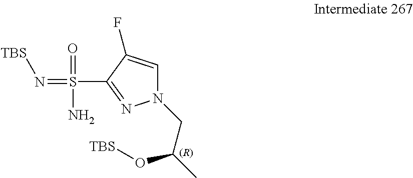

### (R)-N-(tert-butyldimethylsilyl)-1-(2-((tert-butyldimethylsilyl)oxy)propyl)-4-fluoro-1H-pyrazole-3-sulfonamide

### Step 1: (R)-N-(tert-butyldimethylsilyl)-1-(2-((tert-butyldimethylsilyl)oxy)propyl)-4-fluoro-1H-pyrazole-3-sulfonamide

To a stirred solution of (R)-4-fluoro-1-(2-hydroxypropyl)-1H-pyrazole-3-sulfonamide (1.2 g, 5.37 mmol) in THF (30 mL) under nitrogen was added NaH (60% wt., 861 mg, 21.5 mmol) in portions at 0° C. This was followed by the addition of TBSCl (4.86 g, 32.4 mmol) in portions at 0° C. The resulting solution was stirred for 2 h at RT. The reaction was then quenched with 50 mL of water/ice and extracted with 3×50 mL of EtOAc. The organic layers were combined and dried over anhydrous Na₂SO₄ and concentrated under vacuum. The residue was eluted from a silica gel column with EtOAc/PE (1:10). This resulted in 1.8 g (74.1%) of the title compound as a light yellow solid. MS-ESI: 452 (M+1).

### Step 2: N′-(tert-butyldimethylsilyl)-1-((R)-2-((tert-butyldimethylsilyl)oxy)propyl)-4-fluoro-1H-pyrazole-3-sulfonimidamide

To a stirred solution of PPh₃Cl₂ (1.99 g, 5.98 mmol) in CHCl₃ (20 mL) under nitrogen was added DIEA (1.93 g, 14.9 mmol) dropwise at 0° C. over 5 min. The resulting solution was stirred for 20 min at 0° C. This was followed by the addition of (R)-N-(tert-butyldimethylsilyl)-1-(2-((tert-butyldimethylsilyl)oxy)propyl)-4-fluoro-1H-pyrazole-3-sulfonamide (1.35 g, 2.99 mmol) in CHCl₃ (5 mL) dropwise with stirring at 0° C. over 5 min. The resulting solution was stirred for 2 h at 0° C. To the above solution was introduced NH₃ (g) bubbled at 0° C for 10 min. The resulting solution was stirred for 16 h at RT. The solids were filtered out. The filtrate was concentrated under vacuum. The residue was eluted from a silica gel column with EtOAc/PE (1:2). This resulted in 1.0 g (74.2%) of the title compound as a light yellow solid. MS-ESI: 451 (M+1).

### Example 853

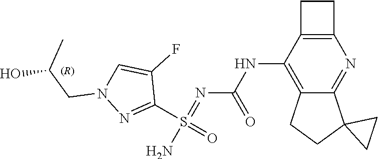

### 4-Fluoro-1-((R)-2-hydroxypropyl)-N′-((1′,5′,6′,7′-tetrahydro-2′H-spiro[cyclopropane-1,3′-dicyclopenta[b,e]pyridin]-8′-yl)carbamoyl)-1H-pyrazole-3-sulfonimidamide (Scheme II)

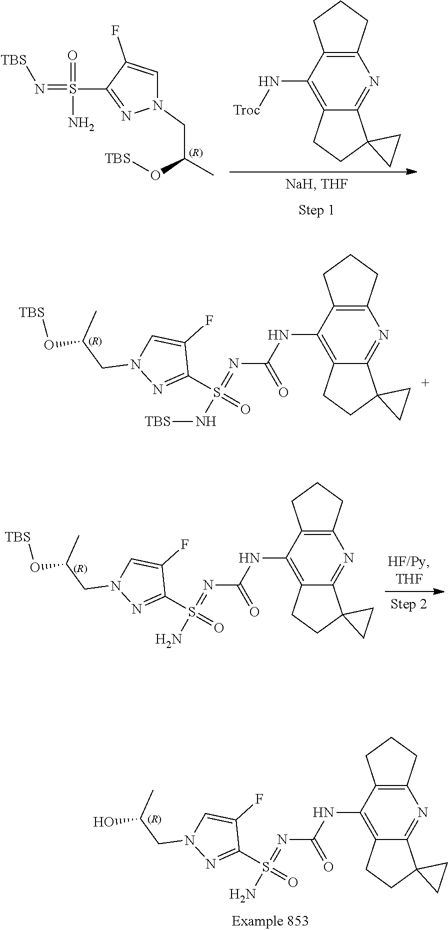

### Step 1: N-(tert-butyldimethylsilyl)-1-((R)-2-((tert-butyldimethylsilypoxy)propyl)-4-fluoro-N′-((1′,5′,6′,7′-tetrahydro-2′H-spiro[cyclopropane-1,3′-dicyclopenta[b,e]pyridin]-8′-yl)carbamoyl)-1H-pyrazole-3-sulfonimidamide and 1-((R)-2-((tert-butyldimethylsilyl)oxy)propyl)-4-fluoro-N′-((1′,5′,6′,7′-tetrahydro-2′H-spiro[cyclopropane-1,3′-dicyclopenta[b,e]pyridin]-8′-yl)carbamoyl)-1H-pyrazole-3-sulfonimidamide

To a stirred solution of N′-(tert-butyldimethylsilyl)-1-((R)-2-((tert-butyldimethylsilyl)oxy)propyl)-4-fluoro-1H-pyrazole-3-sulfonimidamide (1.0 g, 2.22 mmol) in THF (15 mL) under nitrogen was added NaH (60% wt., 178 mg, 4.44 mmol) in portions at 0° C. The resulting solution was stirred for 20 min at 0° C. This was followed by the addition of 2,2,2-trichloroethyl (1′,5′,6′,7′-tetrahydro-2′H-spiro[cyclopropane-1,3′-dicyclopenta[b,e]pyridin]-8′-yl)carbamate (835 mg, 2.22 mmol) in portions at 0° C. The resulting solution was stirred for 3 h at RT. The reaction was then quenched with 50 mL of water/ice and extracted with 3×100 mL of EtOAc. The organic layers were combined and dried over anhydrous Na₂SO₄ and concentrated under vacuum. The residue was eluted from a silica gel column with EtOAc/PE (1:3). This resulted in 751 mg (50%) of N-(tert-butyldimethylsilyl)-1-((R)-2-((tert-butyldimethylsilyl)oxy)propyl)-4-fluoro-N′-((1′,5′,6′,7′-tetrahydro-2′H-spiro[cyclopropane-1,3′-dicyclopenta[b,e]pyridin]-8′-yl)carbamoyl)-1H-pyrazole-3-sulfonimidamide (Bi-TBS product) as a light yellow solid. MS-ESI: 677 (M+1). This also resulted in 500 mg (40%) of 1-((R)-2-((tert-butyldimethylsilyl)oxy)propyl)-4-fluoro-N′-((1′,5′,6′,7′-tetrahydro-2′H-spiro[cyclopropane-1,3′-dicyclopenta[b,e]pyridin]-8′-yl)carbamoyl)-1H-pyrazole-3-sulfonimidamide (mono-TBS product) as a yellow solid which eluted after Bi-TBS product. MS-ESI: 563 (M+1).

### Step 2: 4-fluoro-1-((R)-2-hydroxypropyl)-N′-((1′,5′,6′,7′-tetrahydro-2′H-spiro[cyclopropane-1,3′-dicyclopenta[b,e]pyridin]-8′-yl)carbamoyl)-1H-pyrazole-3-sulfonimidamide

To a stirred solution of N-(tert-butyldimethylsilyl)-1-((R)-2-((tert-butyldimethylsilyl)oxy)propyl)-4-fluoro-N′-((1′,5′,6′,7′-tetrahydro-2′H-spiro[cyclopropane-1,3′-dicyclopenta[b,e]pyridin]-8′-yl)carbamoyl)-1H-pyrazole-3-sulfonimidamide (751 mg, 1.11 mmol) in THF (10 mL) was added HF/Py (70% wt, 160 mg, 5.55 mmol) dropwise at 0° C. The resulting solution was stirred for 3 h at RT. This resulting solution was assigned as A.

To a stirred solution of 1-((R)-2-((tert-butyldimethylsilyl)oxy)propyl)-4-fluoro-N′-((1′,5′,6′,7′-tetrahydro-2′H-spiro[cyclopropane-1,3′-dicyclopenta[b,e]pyridin]-8′-yl)carbamoyl)-1H-pyrazole-3-sulfonimidamide (500 mg, 0.89 mmol) in THF (10 mL) was added HF/Py (70% wt, 51 mg, 1.78 mmol) dropwise at 0° C. The resulting solution was stirred for 3 h at RT. This resulting solution was assigned as B.

The solutions A and B were combined and concentrated under vacuum below 30° C. The residue was purified by Prep-HPLC with the following conditions: XBridge Shield RP18 OBD Column, 30*150 mm, 5 um; Mobile Phase A: Water (10 mM NH₄HCO₃+0.1% NH₃.H₂O), Mobile Phase B: ACN; Flow rate: 60 mL/min; Gradient: 5% B to 25% B over 7 min; Detector 254/220 nm; Rt: 6.58 min. This resulted in 503 mg (56%) of Example 853 as a white solid. MS-ESI: 449 (M+1).

The general synthesis of the below examples may be carried out in accordance with Scheme 4D

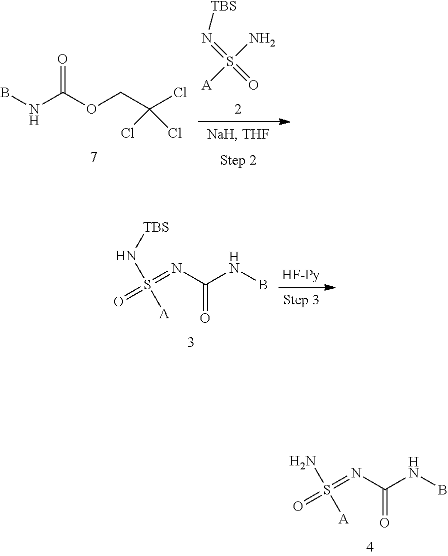

An exemplary synthesis of scheme 4D is provided below for example 9D, and its intermediates (13D, 33D, 62D).

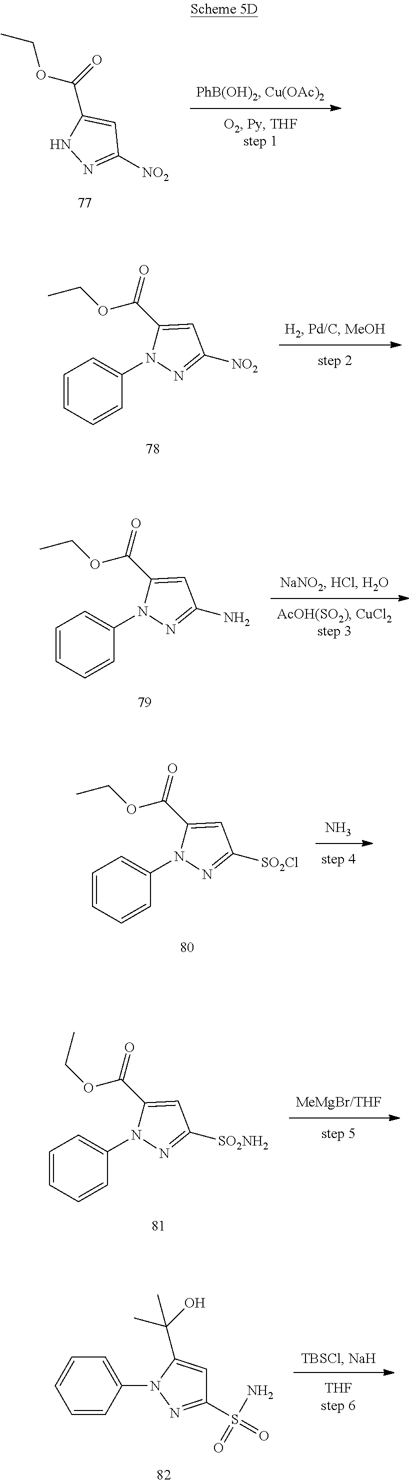

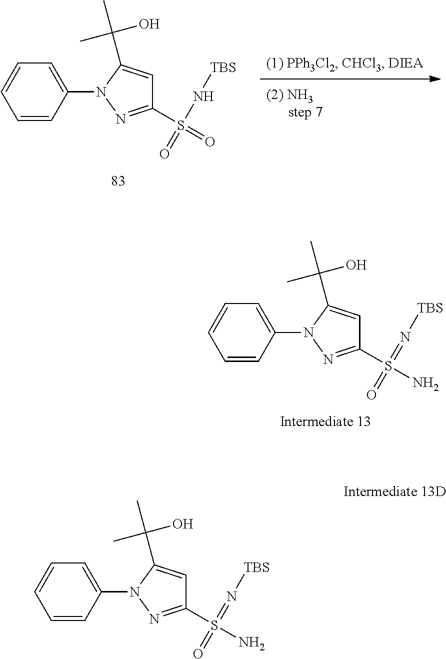

### N′-(tert-butyldimethylsilyl)-5-(2-hydroxypropan-2-yl)-1-phenyl-1H-pyrazole-3-sulfonimidamide

### Step 1: Ethyl 3-nitro-1-phenyl-1H-pyrazole-5-carboxylate

Into a 250-mL round-bottom flask purged and maintained with an inert atmosphere of oxygen, was placed ethyl 3-nitro-1H-pyrazole-5-carboxylate (5 g, 27 mmol) in tetrahydrofuran (150 mL). To the stirred solution was added phenylboronic acid (6.6 g, 54 mmol), Cu(OAc)₂ (7.38 g, 41 mmol) and pyridine (8.54 g, 108 mmol). The resulting solution was stirred overnight at RT. The resulting mixture was concentrated under vacuum. The residue was eluted from silica gel with EtOAc/PE (1:1). This resulted in 3.1 g (44%) of the title compound as an off-white solid. MS-ESI: 262 (M+1).

### Step 2: Ethyl 3-amino-1-phenyl-1H-pyrazole-5-carboxylate

Into a 100-mL round-bottom flask, was placed ethyl 3-nitro-1-phenyl-1H-pyrazole-5-carboxylate (3.92 g, 15 mmol) and methanol (50 mL). To the stirred solution was added Pd/C (wet 10% wt, 400 mg). The flask was evacuated and filled three times with hydrogen. The resulting solution was stirred overnight at RT. The solids were filtered out. The resulting mixture was concentrated under vacuum. This resulted in 2.8 g (81%) of the title compound as a light yellow solid. MS-ESI: 232 (M+1).

### Step 3: Ethyl 3-(chlorosulfonyl)-1-phenyl-1H-pyrazole-5-carboxylate

Into a 100-mL round-bottom flask, was placed ethyl 3-amino-1-phenyl-1H-pyrazole-5-carboxylate (1.8 g, 7.78 mmol) in HCl (6 M, 15 mL). This was followed by the addition of a solution of NaNO₂ (646 mg, 9.36 mmol) in water (2 mL) dropwise with stirring at −10° C. The resulting solution was stirred for 30 min at −10° C. The above mixture was added to a saturated solution of SO₂ in AcOH (20 mL) dropwise with stirring at 0° C. Then to the above was added CuCl₂ (1.05 g, 7.81 mmol). The resulting solution was stirred for 1 h at RT. The reaction was then quenched by the addition of 30 mL of water. The resulting solution was extracted with 3×30 mL of DCM. The organic layers were combined, dried over anhydrous sodium sulfate, and concentrated under vacuum. This resulted in 2.2 g (90%) of the title compound as a light yellow solid.

### Step 4: Ethyl 1-phenyl-3-sulfamoyl-1H-pyrazole-5-carboxylate

Into a 100-mL round-bottom flask, was placed a solution of ethyl 3-(chlorosulfonyl)-1-phenyl-1H-pyrazole-5-carboxylate (2.2 g, 6.99 mmol) in DCM (10 mL). Then to the above was introduced NH₃ gas bubbled at 0° C. for 10 min. The resulting solution was stirred for 2 h at RT. The resulting mixture was concentrated under vacuum. The residue was applied onto silica gel with EtOAc/PE (1:1). This resulted in 1.07 g (52%) of the title compound as a light yellow solid. MS-ESI: 296 (M+1).

### Step 5: 5-(2-Hydroxypropan-2-yl)-1-phenyl-1H-pyrazole-3-sulfonamide

Into a 100-mL 3-necked round-bottom flask purged and maintained with an inert atmosphere of nitrogen, was placed a solution of ethyl 1-phenyl-3-sulfamoyl-1H-pyrazole-5-carboxylate (1.65 g, 5.59 mmol) in tetrahydrofuran (30 mL). This was followed by the addition of MeMgBr (3 M in THF, 18.6 mL) dropwise with stirring at 0° C. The resulting solution was stirred overnight at RT. The reaction was then quenched by the addition of 30 mL of NH₄Cl (sat.). The resulting solution was extracted with 3×30 mL of DCM and the organic layers combined and dried over anhydrous sodium sulfate and concentrated under vacuum. The residue was eluted from silica gel with EtOAc/PE (2:1). This resulted in 1.35 g (86%) of the title compound as a yellow solid. MS-ESI: 282 (M+1).

### Step 6: N-(tert-butyldimethylsilyl)-5-(2-hydroxypropan-2-yl)-1-phenyl-1H-pyrazole-3-sulfonamide

Into a 100-mL round-bottom flask, was placed 5-(2-hydroxypropan-2-yl)-1-phenyl-1H-pyrazole-3-sulfonamide (500 mg, 1.78 mmol) in tetrahydrofuran (10 mL). This was followed by the addition of sodium hydride (60% wt. oil dispersion, 143 mg, 3.58 mmol) in portions at 0° C. Then to the above was added TBSCl (538 mg, 3.57 mmol). The resulting solution was stirred for 2 h at RT. The reaction was then quenched by the addition of 10 mL of water. The resulting solution was extracted with 3×10 mL of DCM. The organic layers were combined, dried over anhydrous sodium sulfate, and concentrated under vacuum. The residue was eluted from silica gel with EtOAc/PE (1:2). This resulted in 660 mg (94%) of the title compound as a light yellow solid. MS-ESI: 396 (M+1).

### Step 7: N′-(tert-butyldimethylsilyl)-5-(2-hydroxypropan-2-yl)-1-phenyl-1H-pyrazole-3-sulfonimidamide

Into a 100-mL 3-necked round-bottom flask purged and maintained with an inert atmosphere of nitrogen, was placed the solution of PPh₃Cl₂ (1.67 g, 5.01 mmol) in chloroform (30 mL). This was followed by the addition of DIEA (1.29 g, 9.98 mmol) dropwise with stirring at RT. The resulting solution was stirred for 10 min at RT and the reaction system was cooled to 0° C. To this was added a solution of N-(tert-butyldimethylsilyl)-5-(2-hydroxypropan-2-yl)-1-phenyl-1H-pyrazole-3-sulfonamide (660 mg, 1.67 mmol) in chloroform (3 mL) dropwise with stirring at 0° C. The resulting solution was stirred for 30 min at 0° C. To the mixture was introduced NH₃ gas bubble for 15 min at 0° C. The resulting solution was stirred for 2 h at RT, after which it was diluted with 30 mL of water. The resulting solution was extracted with 3×30 mL of DCM. The organic layers were combined, dried over anhydrous sodium sulfate, and concentrated under vacuum. The residue was eluted from silica gel with EtOAc/PE (1:1). This resulted in 530 mg (81%) of the title compound as a light yellow solid. MS-ESI: 395 (M+1).

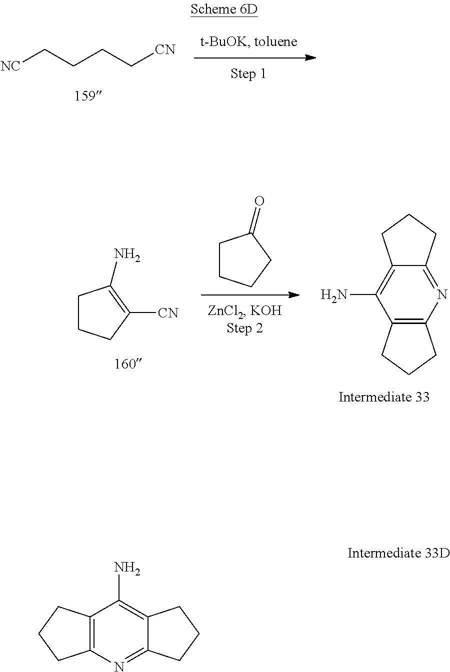 [SMILES: N#CCCCCC#N ; N#CC1=C(N)CCC1 ; O=C1CCCC1 ; Nc1c2c(nc3c1CCC3)CCC2 ; Nc1c2c(nc3c1CCC3)CCC2]

### 1,2,3,5,6,7-Hexahydrodicyclopenta[b,e]pyridin-8-amine

### Step 1: 2-Aminocyclopent-1-ene-1-carbonitrile

Into a 500-mL round-bottom flask purged and maintained with an inert atmosphere of nitrogen, was placed a mixture of adiponitrile (10.8 g, 100 mmol) in toluene (250 mL). The reaction mixture was heated to 65° C., and t-BuOK (112 g, 100 mmol) was added into the solution at 65° C. in portions. The resulting solution was stirred for at 80° C. for 8 h. The reaction mixture was then cooled to RT and quenched by the addition of 200 mL of water/ice. The solids were collected by filtration. The filter cake was washed with water (100 mL) and hexane (200 mL), after which it was dried under an infra-red lamp. This resulted in 9.18 g (85.0%) of the title compound as an off-white solid. MS-ESI: 109 (M+1).

### Step 2: 1,2,3,5,6,7-Hexahydrodicyclopenta[b,e]pyridin-8-amine

Into a 250-mL round-bottom flask purged and maintained with an inert atmosphere of nitrogen, was placed 2-aminocyclopent-1-ene-1-carbonitrile (5.0 g, 46.2 mmol) in xylene (125 mL). To the above solution was added cyclopentanone (7.8 g, 93 mmol) and ZnCl₂ (6.9 g, 51 mmol). The resulting solution was stirred for overnight at 140° C. in an oil bath. The resulting solution was diluted with 150 mL of MeOH, after which a solution of KOH (25 mL, 5.0 M) was dropped into it. The solids were filtered out. The resulting mixture was concentrated. The residue was dissolved in 250 mL of EtOAc. The solids were collected by filtration. This resulted in 4.2 g (52%) of the title compound as a brown solid. MS-ESI: 175 (M+1).

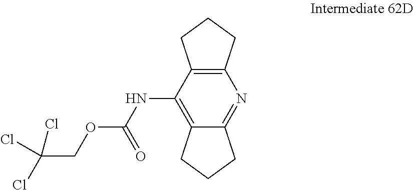 [SMILES: O=C(Nc1c2c(nc3c1CCC3)CCC2)OCC(Cl)(Cl)Cl]

### 2,2,2-Trichloroethyl (1,2,3,5,6,7-hexahydrodicyclopenta[b,e]pyridin-8-yl)carbamate

Into a 1-L round-bottom flask purged and maintained with an inert atmosphere of nitrogen, was placed 1,2,3,5,6,7-hexahydrodicyclopenta[b,e]pyridin-8-amine (6.7 g, 38 mmol) in THF (500 mL). To the above solution was added DIEA (9.92 g, 76.9 mmol) dropwise at RT. Then 2,2,2-trichloroethyl chloroformate (16 g, 76.9 mmol) was dropped into the reaction solution at 0° C. The resulting solution was stirred for 16 h at RT. The resulting mixture was concentrated. The residue was eluted from silica gel with EtOAc/hexane (1:4). This resulted in 7.3 g (54.4%) of the title compound as a yellow solid. MS-ESI: 349/351 (M+1).

### Example 9D

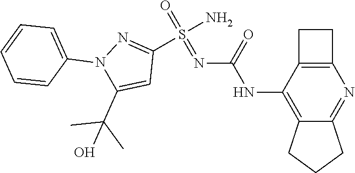 [SMILES: CC(C)(O)c1cc(S(N)(=O)=NC(=O)Nc2c3c(nc4c2CC4)CCC3)nn1-c1ccccc1]

### N′-((1,2,3,5,6,7-hexahydrodicyclopenta[b,e]pyridin-8-yl)carbamoyl)-5-(2-hydroxypropan-2-yl)-1-phenyl-1H-pyrazole-3-sulfonimidamide (scheme 2)

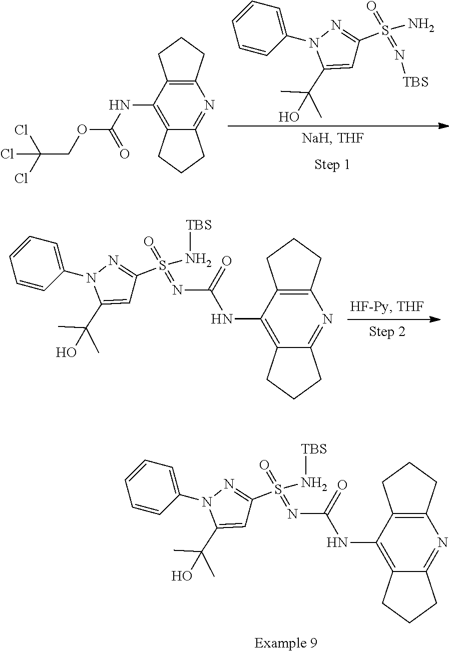

### Step 1: N-(tert-butyldimethylsilyl)-N′-((1,2,3,5,6,7-hexahydrodicyclopenta[b,e]pyridin-8-yl)carbamoyl)-5-(2-hydroxypropan-2-yl)-1-phenyl-1H-pyrazole-3-sulfonimidamide

Into a 50-mL round-bottom flask purged and maintained with an inert atmosphere of nitrogen, was placed N-(tert-butyldimethylsilyl)-5-(2-hydroxypropan-2-yl)-1-phenyl-1H-pyrazole-3-sulfonoimidamide (160 mg, 0.41 mmol) in THF (10 mL). To the stirred solution was added NaH (60% wt. oil dispersion, 48.7 mg, 1.22 mmol) at 0° C. The resulting solution was stirred for 10 min at RT. Then 2,2,2-trichloroethyl (1,2,3,5,6,7-hexahydrodicyclopenta[b,e]pyridin-8-yl)carbamate (142 mg, 0.41 mmol) was added to the reaction solution. The resulting solution was allowed to react with stirring for an additional 2 h while the temperature was maintained at 40° C. in an oil bath. The reaction was then quenched by the addition of 10 mL of H₂O. The resulting solution was extracted with 5×20 mL of EtOAc and the organic layers was combined and concentrated. The residue was purified using TLC with DCM/MeOH=10:1. This resulted in 230 mg (95.4%) of the title compound as a light yellow solid. MS-ESI: 595 (M+1).

### Step 2: N′-((1,2,3,5,6,7-hexahydrodicyclopenta[b,e]pyridin-8-yl)carbamoyl)-5-(2-hydroxypropan-2-yl)-1-phenyl-1H-pyrazole-3-sulfonimidamide

Into a 50-mL round-bottom flask, was placed N-(tert-butyldimethylsilyl)-N′-((1,2,3,4,5,6,7-hexahydrodicyclopenta[b,e]pyridin-8-yl)carbamoyl)-5-(2-hydroxypropan-2-yl)-1-phenyl-1H-pyrazole-3-sulfonimidamide (230 mg, 0.39 mmol) in THF (8 mL). To the above solution was added HF-Pyridine (0.1 mL) dropwise. The resulting solution was stirred for 30 min at RT. The resulting mixture was concentrated. The crude product was purified by Prep-HPLC with the following conditions: Column, XBridge Prep C18 OBD, 19*150 mm 5 um; mobile phase, water (10 mM NH₄HCO₃+0.1% NH₃.H₂O) and ACN (10% to 54% gradient over 6 min); Detector, UV, 210/254 nm. This resulted in 102 mg (53.8%) of Example 9 as an off-white solid. MS-ESI: 481 (M+1). ¹H NMR (400 MHz, DMSO-d₆) δ 8.74 (s, 1H), 7.58 (s, 2H), 7.52 (s, 5H), 6.75 (s, 1H), 5.43 (s, 1H), 2.79 (t, J=7.6 Hz, 4H), 2.71 (t, J=7.5 Hz, 4H), 2.00-1.80 (m, 4H), 1.34 (s, 6H).

TABLE 58C
Examples in the following table were prepared using similar conditions as described
in the synthesis of Example 9D from appropriate starting materials.

| Example | | | Exact Mass |
|---|---|---|---|
| # | Structure | IUPAC Name | [M + H]⁺ |
| 873 | 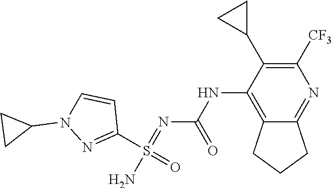 [SMILES: NS(=O)(=NC(=O)Nc1c2c(nc(C(F)(F)F)c1C1CC1)CCC2)c1ccn(C2CC2)n1] | 1-Cyclopropyl-N′-((3-cyclopropyl- 2-(trifluoromethyl)-6,7-dihydro- 5H-cyclopenta[b]pyridin-4- yl)carbamoyl)-1H-pyrazole-3- sulfonimidamide | 455 |
| 874 | 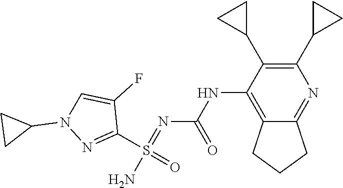 [SMILES: NS(=O)(=NC(=O)Nc1c2c(nc(C3CC3)c1C1CC1)CCC2)c1nn(C2CC2)cc1F] | 1-Cyclopropyl-N′-((2,3- dicyclopropyl-6,7-dihydro-5H- cyclopenta[b]pyridin-4- yl)carbamoyl)-4-fluoro-1H- pyrazole-3-sulfonimidamide | 445 |
| 875 | 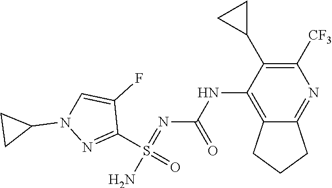 [SMILES: NS(=O)(=NC(=O)Nc1c2c(nc(C(F)(F)F)c1C1CC1)CCC2)c1nn(C2CC2)cc1F] | 1-Cyclopropyl-N′-((3-cyclopropyl- 2-(trifluoromethyl)-6,7-dihydro- 5H-cyclopenta[b]pyridin-4- yl)carbamoyl)-4-fluoro-1H- pyrazole-3-sulfonimidamide | 473 |
| 876 | 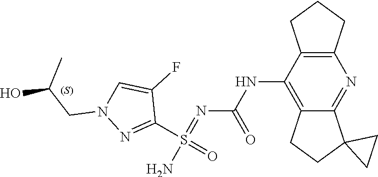 | 4-Fluoro-1-((S)-2-hydroxypropyl)- N′-((1′,5′,6′,7′-tetrahydro-2′H- spiro[cyclopropane-1,3′- dicyclopenta[b,e]pyridin]-8′- yl)carbamoyl)-1H-pyrazole-3- sulfonimidamide | 449 |
| 877 | 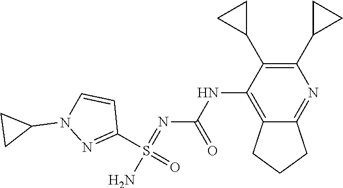 [SMILES: NS(=O)(=NC(=O)Nc1c2c(nc(C3CC3)c1C1CC1)CCC2)c1ccn(C2CC2)n1] | 1-Cyclopropyl-N′-((2,3- dicyclopropyl-6,7-dihydro-5H- cyclopenta[b]pyridin-4- yl)carbamoyl)-1H-pyrazole-3- sulfonimidamide | 427 |

TABLE 71C
Examples in the following table were obtained from chiral HPLC resolutions of
racemic and diastereomeric mixture examples described above. The chiral column and eluents
are listed in the table. As a convention, the faster-eluting enantiomer is always listed first in the
table followed by the slower-eluting enantiomer of the pair. The symbol * at a chiral center
denotes that this chiral center has been resolved and the absolute stereochemistry at that center
has not been determined. Assigned stereochemistry in compound names are tentative.

| Ex. | | | | | LC-MS |
|---|---|---|---|---|---|
| # | Structure | IUPAC Name | Column | Eluents | [M + H]⁺ |
| 878 | 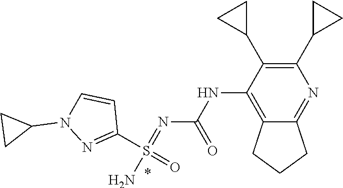 [SMILES: NS(=O)(=NC(=O)Nc1c2c(nc(C3CC3)c1C1CC1)CCC2)c1ccn(C2CC2)n1] | (R) or (S)-1-cyclopropyl- N′-((2,3-dicyclopropyl- 6,7-dihydro-5H- cyclopenta[b]pyridin-4- yl)carbamoyl)-1H- pyrazole-3- sulfonimidamide | CHIRALPAK ID, 2.0*25 cm, 5 um | 40% EtOH in Hex (0.1% FA) | 427 |
| 879 | 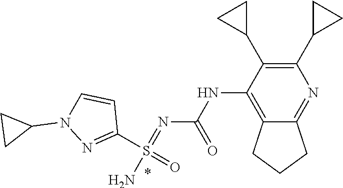 [SMILES: NS(=O)(=NC(=O)Nc1c2c(nc(C3CC3)c1C1CC1)CCC2)c1ccn(C2CC2)n1] | (S) or (R)-1-cyclopropyl- N′-((2,3-dicyclopropyl- 6,7-dihydro-5H- cyclopenta[b]pyridin-4- yl)carbamoyl)-1H- pyrazole-3- sulfonimidamide | CHIRALPAK ID, 2.0*25 cm, 5 um | 40% EtOH in Hex (0.1% FA) | 427 |
| 880 | 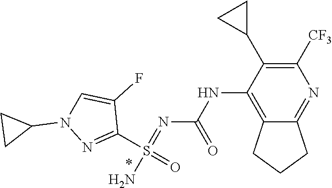 [SMILES: NS(=O)(=NC(=O)Nc1c2c(nc(C(F)(F)F)c1C1CC1)CCC2)c1nn(C2CC2)cc1F] | (R) or (S)-1-cyclopropyl- N′-((3-cyclopropyl-2- (trifluoromethyl)-6,7- dihydro-5H-cyclopenta [b]pyridin-4-yl) carbamoyl)-4-fluoro- 1H-pyrazole-3- sulfonimidamide | CHIRALPAK ID, 2.0*25 cm, 5 um | 40% EtOH in Hex (0.1% FA) | 473 |
| 881 | 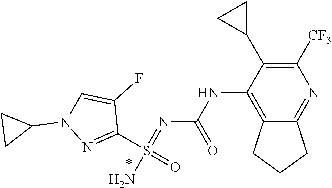 [SMILES: NS(=O)(=NC(=O)Nc1c2c(nc(C(F)(F)F)c1C1CC1)CCC2)c1nn(C2CC2)cc1F] | (S) or (R)-1-cyclopropyl- N′-((3-cyclopropyl-2- (trifluoromethyl)-6,7- dihydro-5H-cyclopenta [b]pyridin-4-yl) carbamoyl)-4-fluoro- 1H-pyrazole-3- sulfonimidamide | CHIRALPAK ID, 2.0*25 cm, 5 um | 40% EtOH in Hex (0.1% FA) | 473 |
| 882 | 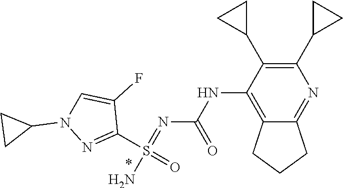 [SMILES: NS(=O)(=NC(=O)Nc1c2c(nc(C3CC3)c1C1CC1)CCC2)c1nn(C2CC2)cc1F] | (R) or (S)-1-cyclopropyl- N′-((2,3-dicyclopropyl- 6,7-dihydro-5H- cyclopenta[b]pyridin-4- yl)carbamoyl)-4-fluoro- 1H-pyrazole-3- sulfonimidamide | CHIRALPAK IC, 2*25 cm, 5 um | 50% EtOH in Hex (0.1% FA) | 445 |
| 883 | 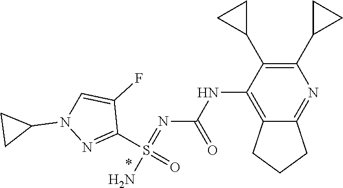 [SMILES: NS(=O)(=NC(=O)Nc1c2c(nc(C3CC3)c1C1CC1)CCC2)c1nn(C2CC2)cc1F] | (S) or (R)-1-cyclopropyl- N′-((2,3-dicyclopropyl- 6,7-dihydro-5H- cyclopenta[b]pyridin-4- yl)carbamoyl)-4-fluoro- 1H-pyrazole-3- sulfonimidamide | CHIRALPAK IC, 2*25 cm, 5 um | 50% EtOH in Hex (0.1% FA) | 445 |
| 884 | 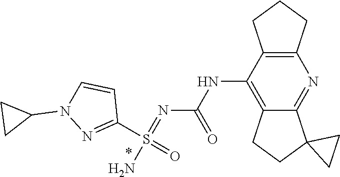 [SMILES: NS(=O)(=NC(=O)Nc1c2c(nc3c1CCC31CC1)CCC2)c1ccn(C2CC2)n1] | (R) or (S)-1-cyclopropyl- N′-((1′,5′,6′,7′-tetrahydro- 2′H-spiro[cyclopropane- 1,3′-dicyclopenta[b,e] pyridin]-8′-yl)carbamoyl)- 1H-pyrazole-3- sulfonimidamide | CHIRALPAK IC, 3*25 cm, 5 um | 50% EtOH in Hex (0.1% FA) | 413 |
| 885 | 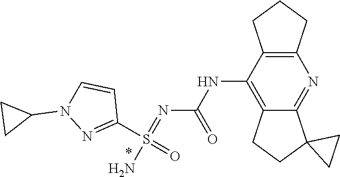 [SMILES: NS(=O)(=NC(=O)Nc1c2c(nc3c1CCC31CC1)CCC2)c1ccn(C2CC2)n1] | (S) or (R)-1-cyclopropyl- N′-((1′,5′,6′,7′-tetrahydro- 2′H-spiro[cyclopropane- 1,3′-dicyclopenta[b,e] pyridin]-8′-yl)carbamoyl)- 1H-pyrazole-3- sulfonimidamide | CHIRALPAK IC, 3*25 cm, 5 um | 50% EtOH in Hex (0.1% FA) | 413 |
| 886 | 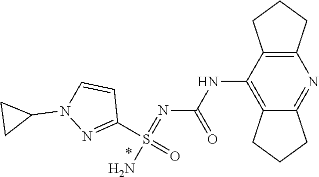 [SMILES: NS(=O)(=NC(=O)Nc1c2c(nc3c1CCC3)CCC2)c1ccn(C2CC2)n1] | (R) or (S)-1-cyclopropyl- N′-((1,2,3,5,6,7- hexahydrodicyclopenta [b,e]pyridin-8-yl) carbamoyl)-1H-pyrazole- 3-sulfonimidamide | CHIRALPAK IG, 2*25 cm, 5 um | 30% EtOH in MTBE (2 mM NH₃— MeOH) | 387 |
| 887 | 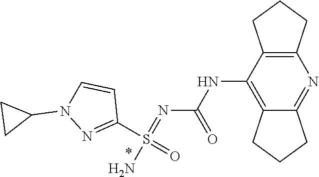 [SMILES: NS(=O)(=NC(=O)Nc1c2c(nc3c1CCC3)CCC2)c1ccn(C2CC2)n1] | (S) or (R)-1-cyclopropyl- N′-((1,2,3,5,6,7- hexahydrodicyclopenta [b,e]pyridin-8-yl) carbamoyl)-1H-pyrazole- 3-sulfonimidamide | CHIRALPAK IG, 2*25 cm, 5 um | 30% EtOH in MTBE (2 mM NH₃— MeOH) | 387 |
| 888 | 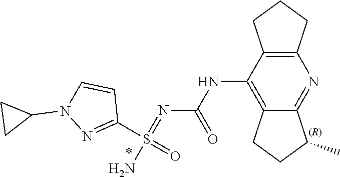 | (R) or (S)-1-cyclopropyl- N′-(((R)-3- methyl-1,2,3,5,6,7- hexahydrodicyclopenta [b,e]pyridin-8-yl) carbamoyl)-1H-pyrazole- 3-sulfonimidamide | Chiralpak ID, 2*25 cm, 5 um | 10% EtOH in Hex: DCM = 3:1 (10 mM NH₃— MeOH) | 401 |
| 889 | 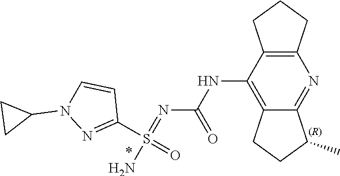 | (S) or (R)-1-cyclopropyl- N′-(((R)-3- methyl-1,2,3,5,6,7- hexahydrodicyclopenta [b,e]pyridin-8-yl) carbamoyl)-1H-pyrazole- 3-sulfonimidamide | Chiralpak ID, 2*25 cm, 5 um | 10% EtOH in Hex: DCM = 3:1 (10 mM NH₃— MeOH) | 401 |
| 890 | 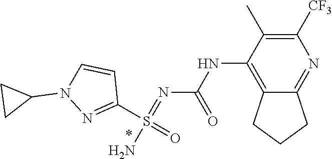 [SMILES: Cc1c(C(F)(F)F)nc2c(c1NC(=O)N=S(N)(=O)c1ccn(C3CC3)n1)CCC2] | (R) or (S)-1-cyclopropyl- N′-((3-methyl-2- (trifluoromethyl)-6,7- dihydro-5H-cyclopenta [b]pyridin-4-yl) carbamoyl)-1H-pyrazole- 3-sulfonimidamide | Chiralpak ID, 2*25 cm, 5 um | 30% EtOH in Hex (0.1% FA) | 429 |
| 891 | 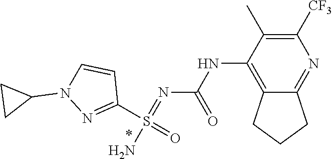 [SMILES: Cc1c(C(F)(F)F)nc2c(c1NC(=O)N=S(N)(=O)c1ccn(C3CC3)n1)CCC2] | (S) or (R)-1-cyclopropyl- N′-((3-methyl-2- (trifluoromethyl)-6,7- dihydro-5H-cyclopenta [b]pyridin-4-yl) carbamoyl)-1H-pyrazole- 3-sulfonimidamide | Chiralpak ID, 2*25 cm, 5 um | 30% EtOH in Hex (0.1% FA) | 429 |
| 892 | 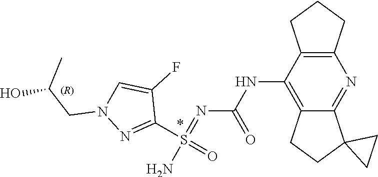 | (R) or (S)-4-fluoro-1- ((R)-2-hydroxypropyl)- N′-((1′,5′,6′,7′- tetrahydro-2′H-spiro [cyclopropane-1,3′- dicyclopenta[b,e] pyridin]-8′-yl) carbamoyl)-1H-pyrazole- 3-sulfonimidamide | CHIRALPAK IH, 2.0*25 cm, 5 um | 50% MeOH (2 mM NH₃— MeOH) in CO₂ | 449 |
| 893 | 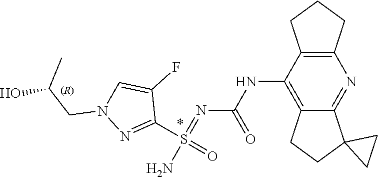 | (S) or (R)-4-fluoro-1- ((R)-2-hydroxypropyl)- N′-((1′,5′,6′,7′- tetrahydro-2′H-spiro [cyclopropane-1,3′- dicyclopenta[b,e] pyridin]-8′-yl) carbamoyl)-1H-pyrazole- 3-sulfonimidamide | CHIRALPAK IH, 2.0*25 cm, 5 um | 50% MeOH (2 mM NH₃— MeOH) in CO₂ | 449 |
| 894 | 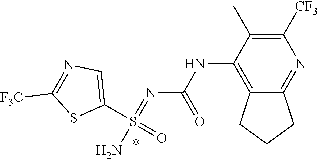 [SMILES: Cc1c(C(F)(F)F)nc2c(c1NC(=O)N=S(N)(=O)c1cnc(C(F)(F)F)s1)CCC2] | (R) or (S)-N′-((3-methyl- 2-(trifluoromethyl)-6,7- dihydro-5H- cyclopenta[b]pyridin-4- yl)carbamoyl)-2- (trifluoromethyl) thiazole-5- sulfonimidamide | CHIRALPAK IE, 2*25 cm, 5 um | 10% EtOH in Hex (0.1% FA) | 474 |
| 895 | 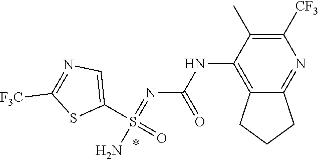 [SMILES: Cc1c(C(F)(F)F)nc2c(c1NC(=O)N=S(N)(=O)c1cnc(C(F)(F)F)s1)CCC2] | (S) or (R)-N′-((3-methyl- 2-(trifluoromethyl)-6,7- dihydro-5H- cyclopenta[b]pyridin-4- yl)carbamoyl)-2- (trifluoromethyl) thiazole-5- sulfonimidamide | CHIRALPAK IE, 2*25 cm, 5 um | 10% EtOH in Hex (0.1% FA) | 474 |
| 896 | 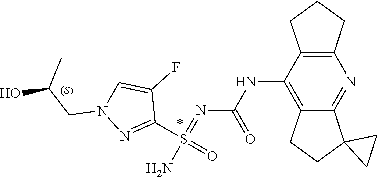 | (R) or (S)-4-fluoro-1- ((S)-2-hydroxypropyl)- N′-((1′,5′,6′,7′- tetrahydro-2′H-spiro [cyclopropane-1,3′- dicyclopenta[b,e] pyridin]-8′-yl) carbamoyl)-1H-pyrazole- 3-sulfonimidamide | CHIRALPAK IC, 2*25 cm, 5 um | 40% EtOH in Hex (0.1% FA) | 449 |
| 897 | 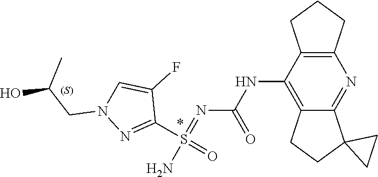 | (S) or (R)-4-fluoro-1- ((S)-2-hydroxypropyl)- N′-((1′,5′,6′,7′- tetrahydro-2′H-spiro [cyclopropane-1,3′- dicyclopenta[b,e] pyridin]-8′-yl) carbamoyl)-1H-pyrazole- 3-sulfonimidamide | CHIRALPAK IC, 2*25 cm, 5 um | 40% EtOH in Hex (0.1% FA) | 449 |
| 898 | 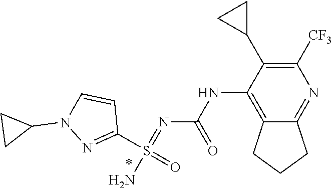 [SMILES: NS(=O)(=NC(=O)Nc1c2c(nc(C(F)(F)F)c1C1CC1)CCC2)c1ccn(C2CC2)n1] | (R) or (S)-1-cyclopropyl- N′-((3-cyclopropyl-2- (trifluoromethyl)-6,7- dihydro-5H-cyclopenta [b]pyridin-4-yl) carbamoyl)-1H-pyrazole- 3-sulfonimidamide | Chiralpak ID, 2*25 cm, 5 um | 30% EtOH in Hex (0.1% FA) | 455 |
| 899 | 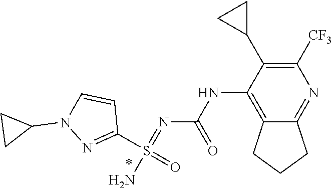 [SMILES: NS(=O)(=NC(=O)Nc1c2c(nc(C(F)(F)F)c1C1CC1)CCC2)c1ccn(C2CC2)n1] | (S) or (RS)-1-cyclopropyl- N′-((3-cyclopropyl-2- (trifluoromethyl)-6,7- dihydro-5H-cyclopenta [b]pyridin-4-yl) carbamoyl)-1H-pyrazole- 3-sulfonimidamide | Chiralpak ID, 2*25 cm, 5 um | 30% EtOH in Hex (0.1% FA) | 455 |

In one embodiment, provided herein is a pharmaceutical composition comprising any NLRP3 antagonist species defined here (for example, a compound or example of Tables B1 and B2), and an anti-TNFα agent disclosed herein. Preferably wherein the anti-TNFα agent is Infliximab, Etanercept, Certolizumab pegol, Golimumab or Adalimumab, more preferably wherein the anti-TNFα agent is Adalimumab.

In one embodiment, provided herein is a pharmaceutical combination of a compound of any NLRP3 antagonist species defined here (for example, a compound or example of Tables B1 and B2), and an anti-TNFα agent. Preferably wherein the anti-TNFα agent is Infliximab, Etanercept, Certolizumab pegol, Golimumab or Adalimumab, more preferably wherein the anti-TNFα agent is Adalimumab.

The following protocol is suitable for testing the activity of the compounds disclosed herein.

1. Experimental procedure
1.1 Cell Culture
1) Culture THP-1 cells in the complete RPMI-1640 medium with 10% FBS at 37° C., 5% CO₂.
2) Passage the cells every 3 days by inoculating 3×10⁵ cells per ml.
1.2 Compound Preparation
   Prepare the 3-fold serial dilution of the compounds with DMSO in a 384-well LDV Microplate using TECAN EVO system to generate the compound source plate with 10 concentrations. Top concentration is 30 mM.
1.3 Cell Preparation
1) Centrifuge THP-1 cells at 350g for 5 min.
2) Re-suspend cells with complete RMPI-1640 medium, and count cells.
3) Seed cells in T225 flask, about 2.5×10⁷ per flask, treat cells with 20 ng/ml PMA (final DMSO concentration<1%).
4) Incubate overnight.
1.4 THP-1 Stimulation
1) Wash adherent THP-1 cells with PBS, and detach cells with 4 ml trypsin for T225 flask.
2) Centrifuge cells at 350 g for 5 min, re-suspend cells with RPMI-1640 containing 2% FBS and count cells with trypan blue.
3) Transfer 50 nl/well the serial dilution of test compound to 384-well plate by Echo; For the high control and first point of CRID3 (MCC950), transfer 165 nl, then backfill to make the DMSO concentration is consistent in all wells, the plate layout is as below.
4) Seed 50k cells in 40 ul RPMI-1640 with 2% FBS per well in 384-well plate.
5) Incubate for 1h at 37° C., 5% CO₂.
6) Prepare 5× gramicidin, add 10 μl per well, the final concentration is 5 μM, incubate for 2 hrs at 37° C., 5% CO₂.
7) Centrifuge at 350 g for 1 min.
8) Pipet 16 μl supernatant by apricot, and transfer into white 384 proxiplate. HC: 100 μM CRID3 (MCC950)+5 μM gramicidin LC: 5 μM Gramicidin.
1.5 IL-1β Detection
1) Homogenize the 5× diluent #5 with a vortex and add 1 volume of stock solution in 4 volumes of distilled water.
2) Thaw 20× stock solution of anti-IL1β-Cryptate-antibody and anti-IL1β XL-antibody. Dilute these two antibodies to 1× with detection buffer #3.
3) Pre-mix the two ready-to-use antibody solutions just prior to use.
4) Dispense 4 ul of pre-mixed Anti-IL1β antibodies working solution into all wells.
5) Seal the plate and incubate overnight at 4° C.
6) Read the cell plate using EnVison and plot Readout vs. the test compound concentration to calculate the IC₅₀.

2. Data Analysis:
1. IC₅₀ of compounds can be calculated using the following formulas
   Formula for IC₅₀

% inhibition=100−100×[HCₐᵥₑ-Readout/(HCₐᵥₑ−LCₐᵥₑ)]
2. Fit the normalized data in a dose-response manner using XLfit, and calculate the compound concentration.

Tables B1 and B2 shows the biological activity of compounds in hTHP-1 assay containing 2% fetal bovine serum: <0.008 μM=“++++++”; ≥0.008 and <0.04 μM=“+++++”; ≥0.04 and <0.2 μM=“++++”; ≥0.2 and <1 μM=“+++”; ≥1 and <5 μM=“++”; ≥5 and <30 μM=“+”.

TABLE B1

| Example | Compound | hTHP-1 | hTHP-1 |
|---|---|---|---|
| # | Number | IC₅₀ | IC₅₀ |
| 1 | 105a | ++++ | 0.0535 |
| 2 | 105b | +++++ | 0.0146 |
| 3 | 106 | ++++ | 0.034 |
| 4 | 104b | ++++ | 0.0597 |
| 5 | 104a | +++ | 0.2359 |
| 6 | 103b | ++++ | 0.0717 |
| 7 | 103a | ++++++ | 0.0068 |
| 8 | 102b | ++++ | 0.0422 |
| 9 | 102a | ++++ | 0.1035 |
| 10 | 101b | ++ | 2.4372 |
| 11 | 101a | ++++ | 0.0906 |
| 12 | 108a | ++++ | 0.4186 |
| 13 | 108b | ++++ | 0.112 |
| 14 | 110a | ++++ | 0.9734 |
| 15 | 110b | +++ | 0.2086 |
| 16 | 109a | +++ | 0.2041 |
| 17 | 109b | ++ | 1.2406 |
| 18 | 109c | ++++ | 0.0803 |
| 19 | 109d | +++ | 0.7051 |
| 20 | | ++ | 2.7474 |
| 21 | | ++++ | 0.0508 |
| 22 | | ++++ | 0.0792 |
| 23 | | +++++ | 0.0389 |
| 24 | | +++++ | 0.0097 |
| 25 | | +++++ | 0.0275 |
| 26 | | +++++ | 0.0135 |
| 27 | | ++++ | 0.0928 |
| 28 | | +++++ | 0.0308 |
| 29 | | ++++ | 0.0582 |
| 30 | | +++++ | 0.0167 |
| 31 | | +++++ | 0.0338 |
| 32 | | ++++ | 0.0795 |
| 33 | | ++ | 1.5314 |
| 34 | | +++ | 0.3548 |

TABLE B2

| | hTHP-1 | hTHP-1 |
|---|---|---|
| Example | IC₅₀ in | IC₅₀ in |
| # | 10% FBS | 10% FBS |
| 873 | ++++ | 0.128 |
| 874 | ++++ | 0.0429 |
| 875 | ++++ | 0.0403 |
| 876 | ++++++ | 0.00469 |
| 877 | ++++ | 0.154 |
| 878 | ++++ | 0.088 |
| 879 | ++ | 4.43 |
| 880 | ++++ | 0.0539 |
| 881 | ++ | 1.41 |
| 882 | ++++ | 0.102 |
| 883 | ++ | 1.25 |
| 884 | ++++ | 0.0533 |
| 885 | ++ | 2.26 |
| 886 | +++ | 0.27 |
| 887 | + | 5.88 |
| 888 | ++++ | 0.0494 |
| 889 | ++ | 1.67 |
| 890 | ++++ | 0.108 |
| 891 | + | 6.51 |
| 892 | +++++ | 0.0127 |
| 893 | +++ | 0.679 |
| 894 | ND | |
| 895 | ND | |
| 896 | +++++ | 0.0136 |
| 897 | +++ | 0.31 |
| 898 | ++++ | 0.114 |
| 899 | + | 9.96 |

### Study Example 1

The CARD8 gene is located within the inflammatory bowel disease (IBD) 6 linkage region on chromosome 19. CARD8 interacts with NLRP3, and Apoptosis-associated Speck-like protein to form a caspase-1 activating complex termed the NLRP3 inflammasome. The NLRP3 inflammasome mediates the production and secretion of interleukin-1β, by processing pro-IL-1β into mature secreted IL-1β. In addition to its role in the inflammasome, CARD8 is also a potent inhibitor of nuclear factor NF-κB. NF-κB activation is essential for the production of pro-IL-1ϑ. Since over-production of IL-1β and dyregulation of NF-κB are hallmarks of Crohn's disease, CARD8 is herein considered to be a risk gene for inflammatory bowel disease. A significant association of CARD8 with Crohn's disease was detected in two British studies with a risk effect for the minor allele of the non-synonymous single-nucleotide polymorphism (SNP) of a C allele at rs2043211. This SNP introduces a premature stop codon, resulting in the expression of a severely truncated protein. This variant CARD8 protein is unable to suppress NF-κB activity, leading to constitutive production of pro-IL-1β, which is a substrate for the NLRP3 inflammasome. It is believed that a gain-of-function mutation in an NLRP3 gene (e.g., any of the gain-of-function mutations described herein, e.g., any of the gain-of-function mutations in an NLRP3 gene described herein) combined with a loss-of-function mutation in a CARD8 gene (e.g., a C allele at rs2043211) results in the development of diseases related to increased NLRP3 inflammasome expression and/or activity. Patients having, e.g., a gain-of-function mutation in an NLRP3 gene and/or a loss-of-function mutation in a CARD8 gene are predicted to show improved therapeutic response to treatment with an NLRP3 antagonist.

A study is designed to determine: whether NLRP3 antagonists inhibit inflammasome function and inflammatory activity in cells and biopsy specimens from patients with Crohn's disease or ulcerative colitis; and whether the specific genetic variants identify patients with Crohn's disease or ulcerative colitis who are most likely to respond to treatment with an NLRP3 antagonist.

The secondary objectives of this study are to: determine if an NLRP3 antagonist reduces inflammasome activity in Crohn's disease and ulcerative biopsy samples (comparing Crohn's disease and ulcerative colitis results with control patient results); determine if an NLRP3 antagonist reduced inflammatory cytokine RNA and protein expression in Crohn's disease and ulcerative colitis samples; determine if baseline (no ex vivo treatment) RNA levels of NLRP3, ASC, and IL-1β are greater in biopsy samples from patients with anti-TNFα agent resistance status; and stratify the results according to presence of specific genetic mutations in genes encoding ATG16L1, NLRP3, and CARD8 (e.g., any of the mutations in the ATG16L1 gene, NLRP3 gene, and CARD8 gene described herein).

### Methods

Evaluation of baseline expression of NLRP3 RNA and quantify inhibition of inflammasome activity by an NLRP3 antagonist in biopsies of disease tissue from patients with Crohn's disease and ulcerative colitis.
Determine if NLRP3 antagonist treatment reduces the inflammatory response in biopsies of disease from patients with Crohn's disease based on decreased expression of inflammatory gene RNA measured with Nanostring.
Sequence patient DNA to detect specific genetic mutations in the ATG16L1 gene, NLRP3 gene, and CARD8 gene (e.g., any of the exemplary mutations in these genes described herein) and then stratify the results of functional assays according to the presence of these genetic mutations.

### Experimental Design

Human subjects and tissue:
   Endoscopic or surgical biopsies from areas of disease in patients with Crohn's disease and ulcerative colitis who are either anti-TNFα treatment naïve or resistant to anti-TNFα treatment; additionally biopsies from control patients (surveillance colonoscopy or inflammation-free areas from patients with colorectal cancer) are studied.
Ex vivo Treatment Model:
   Organ or LPMC culture as determined appropriate
Endpoints to be measured:
   Before ex vivo treatment—NLRP3 RNA level
   After ex vivo treatment—inflammasome activity (either processed IL-1β, processed caspase-1, or secreted IL-1β); RNA for inflammatory cytokines (Nanostring); viable T cell number and/or T cell apoptosis.
Data Analysis Plan:
   Determine if NLRP3 antagonist treatment decreases processed IL-1β, processed caspase-1 or secreted IL-1β, and inflammatory cytokine RNA levels.
   Stratify response data according to treatment status at biopsy and the presence of genetic mutations in the NLRP3 gene, CARD8 gene, and ATG16L1 gene (e.g., any of the exemplary genetic mutations of these genes described herein).

### Study Example 2. Treatment of Anti-TNFα Resistant Patients with NLRP3 Antagonists

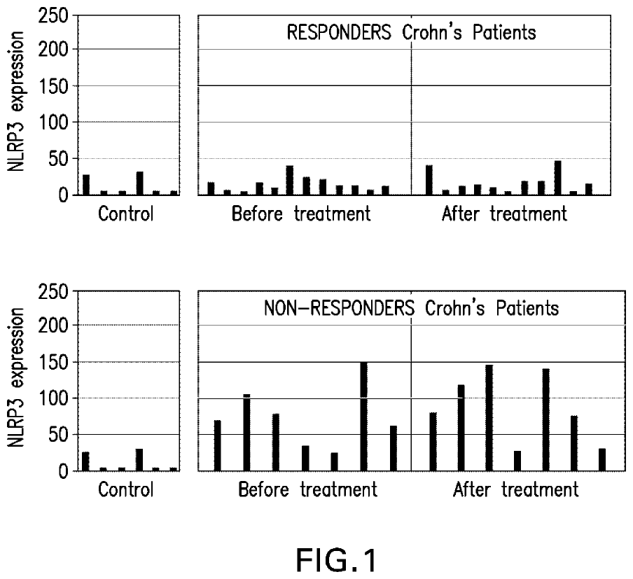

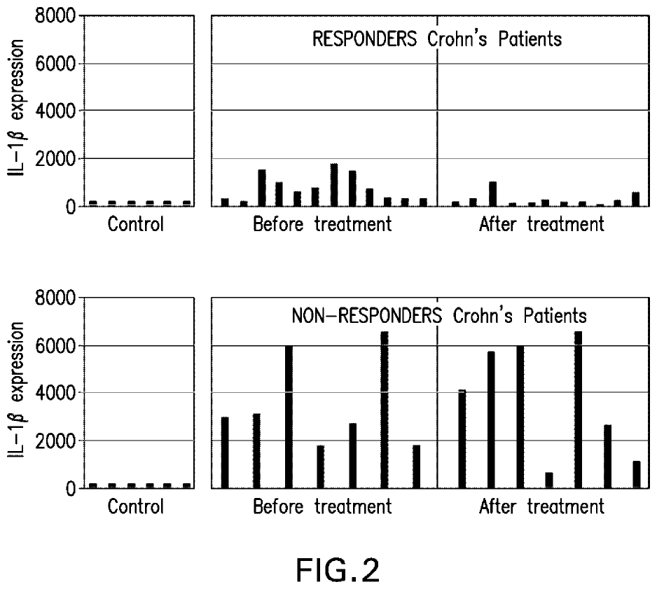

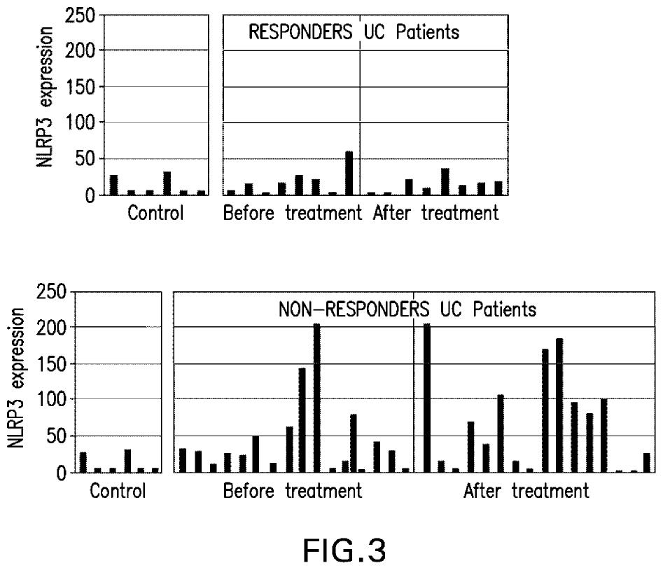

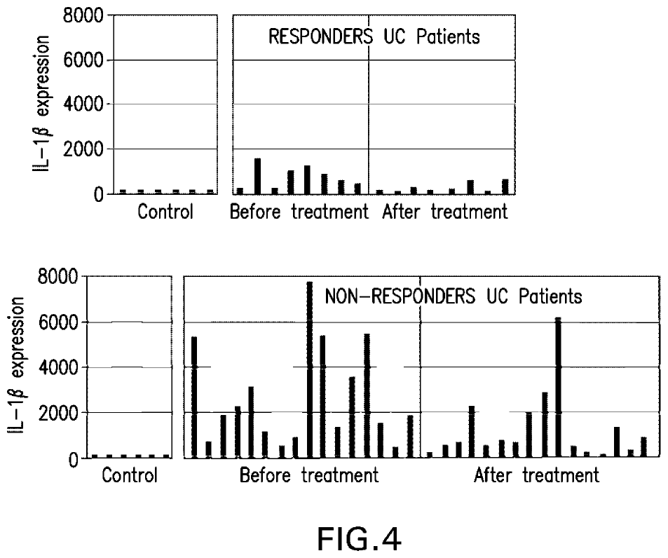

PLoS One 2009 Nov. 24; 4(11):e7984, describes that mucosal biopsies were obtained at endoscopy in actively inflamed mucosa from patients with Ulcerative Colitis, refractory to corticosteroids and/or immunosuppression, before and 4-6 weeks after their first infliximab (an anti-TNFα agent) infusion and in normal mucosa from control patients. The patients in this study were classified for response to infliximab based on endoscopic and histologic findings at 4-6 weeks after first infliximab treatment as responder or non-responder. Transcriptomic RNA expression levels of these biopsies were accessed by the inventors of the invention disclosed herein from GSE 16879, the publically available Gene Expression Omnibus (https://www.ncbi.nlm.nih.gov/geo/geo2r/?acc=GSE16879). Expression levels of RNA encoding NLRP3 and IL- 1β were determined using GEO2R (a tool available on the same website), based on probe sets 207075_at and 205067_at, respectively. It was surprisingly found that in Crohn's disease patients that are non-responsive to the infliximab (an anti-TNFα agent) have higher expression of NLRP3 and IL-1β RNA than responsive patients (FIGS. 1 and 2). Similar surprising results of higher expression of NLRP3 and IL-1β RNA in UC patients that are non-responsive to infliximab (an anti-TNFα agent) compared to infliximab (an anti-TNFα agent) responsive patients (FIGS. 3 and 4) were found.

Said higher levels of NLRP3 and IL-1β RNA expression levels in anti-TNFα agent non-responders, is hypothesised herein to lead to NLRP3 activation which in turns leads of release of IL-1β that induces IL-23 production, leading to said resistance to anti-TNFα agents. Therefore, treatment of Crohn's and UC anti-TNFα non-responders with an NLRP3 antagonist would prevent this cascade, and thus prevent development of non-responsiveness to anti-TNFα agents. Indeed, resistance to anti-TNFα agents is common in other inflammatory or autoimmune diseases. Therefore, use of an NLRP3 antagonist for the treatment of inflammatory or autoimmune diseases will block the mechanism leading to non-responsiveness to anti-TNFα agents. Consequently, use of NLRP3 antagonists will increase the sensitivity of patients with inflammatory or autoimmune diseases to anti-TNFα agents, resulting in a reduced dose of anti-TNFα agents for the treatment of these diseases. Therefore, a combination of an NLRP3 antagonist and an anti-TNFα agent can be used in the treatment of diseases wherein TNFα is overexpressed, such as inflammatory or autoimmune diseases, to avoid such non-responsive development of patients to anti-TNFα agents. Preferably, this combination treatment can be used in the treatment of IBD, for example Crohn's disease and UC.

Further, use of NLRP3 antagonists offers an alternative to anti-TNFα agents for the treatment of diseases wherein TNFα is overexpressed. Therefore, NLRP3 antagonists offers an alternative to anti-TNFα agents inflammatory or autoimmune diseases, such as IBD (e.g. Crohn's disease and UC).

Systemic anti-TNFα agents are also known to increase the risk of infection. Gut restricted NLRP3 antagonists, however, offers a gut targeted treatment (i.e. non-systemic treatment), preventing such infections. Therefore, treatment of TNFα gut diseases, such as IBD (i.e. Crohn's disease and UC), with gut restricted NLRP3 antagonists has the additional advantage of reducing the risk of infection compared to anti-TNFα agents.

Proposed Experiment:

Determine the expression of NLRP3 and caspase-1 in LPMCs and epithelial cells in patients with non-active disease, in patients with active disease, in patients with active disease resistant to corticosteroids, patients with active disease resistant to TNF-blocking agents. The expression of NLRP3 and caspase-1 in LPMCs and epithelial cells will be analyzed by RNAScope technology. The expression of active NLRP3 signature genes will be analyzed by Nanostring technology. A pilot analysis to determine feasibility will be performed with 5 samples from control, 5 samples from active CD lesions, and 5 samples from active UC lesions.

### Study Example 3

It is presented that NLRP3 antagonists reverse resistance to anti-TNF induced T cell depletion/apoptosis in biopsy samples from IBD patients whose disease is clinically considered resistant or unresponsive to anti-TNF therapy.

A study is designed to determine: whether NLRP3 antagonists inhibit inflammasome function and inflammatory activity in cells and biopsy specimens from patients with Crohn's disease or ulcerative colitis; and whether an NLRP3 antagonist will synergize with anti-TNFα therapy in patients with Crohn's disease or ulcerative colitis.

The secondary objectives of this study are to: determine if an NLRP3 antagonist reduces inflammasome activity in Crohn's disease and ulcerative biopsy samples (comparing Crohn's disease and ulcerative colitis results with control patient results); determine if an NLRP3 antagonist reduced inflammatory cytokine RNA and protein expression in Crohn's disease and ulcerative colitis samples; determine if an NLRP3 antagonist in the absence of co-treatment with anti-TNFα antibody induces T cell depletion in Crohn's disease and ulcerative colitis biopsy samples; and determine if baseline (no ex vivo treatment) RNA levels of NLRP3, ASC, and IL-1β are greater in biopsy samples from patients with anti-TNFα agent resistance status.

### Methods

Evaluation of baseline expression of NLRP3 RNA and quantify inhibition of inflammasome activity by an NLRP3 antagonist in biopsies of disease tissue from patients with Crohn's disease and ulcerative colitis.
Determine if there is synergy between an NLRP3 antagonist and anti-TNF antibody with respect to effects on T cell depletion/apoptosis in biopsies of disease from patients with Crohn's disease and ulcerative colitis.
Determine if NLRP3 antagonist treatment reduces the inflammatory response in biopsies of disease from patients with Crohn's disease based on decreased expression of inflammatory gene RNA measured with Nanostring.

### Experimental Design

Human subjects and tissue:
   Endoscopic or surgical biopsies from areas of disease in patients with Crohn's disease and ulcerative colitis who are either anti-TNFα treatment naïve or resistant to anti-TNFα treatment; additionally biopsies from control patients (surveillance colonoscopy or inflammation-free areas from patients with colorectal cancer) are studied.
Ex Vivo Treatment Model:
   Organ or LPMC culture as determined appropriate
Ex vivo Treatments:

NLRP3 antagonist (2 concentrations), negative control (vehicle), positive control (caspase-1 inhibitor) each in the presence or absence of anti-TNF antibody at a concentration appropriate to distinguish differences in the T cell apoptotic between biopsies from anti-TNF resistant and anti-TNF-sensitive Crohn's disease patients. Each treatment condition is evaluated in a minimum in duplicate samples.
Endpoints to be measured:
   Before ex vivo treatment—NLRP3 RNA level
   After ex vivo treatment—inflammasome activity (either processed IL-1β, processed caspase-1, or secreted IL-1β; RNA for inflammatory cytokines (Nanostring); viable T cell number and/or T cell apoptosis.
Data Analysis Plan:
   Determine if NLRP3 antagonist co-treatment increases T cell apoptosis/deletion in response to anti-TNF.
   Determine if the level of NLRP3 RNA expression is greater in TNF-resistant Crohn's disease and ulcerative colitis samples compared to anti-TNF treatment-naïve samples.
   Determine if NLRP3 antagonist treatment decreases processed IL-1β, processed caspase-1 or secreted IL-10, and inflammatory cytokine RNA levels.

Biological Assay—Nigericin-Stimulated IL-1β Secretion Assay in THP-1 Cells

Monocytic THP-1 cells (ATCC: TIB-202) were maintained according to providers' instructions in RPMI media (RPMI/Hepes+10% fetal bovine serum+Sodium Pyruvate+0.05 mM Beta-mercaptoethanol (1000× stock)+Pen-Strep). Cells were differentiated in bulk with 0.5 μM phorbol 12-myristate 13-acetate (PMA; Sigma #P8139) for 3 hours, media was exchanged, and cells were plated at 50,000 cells per well in a 384-well flat-bottom cell culture plates (Greiner, #781986), and allowed to differentiate overnight. Compound in a 1:3.16 serial dilution series in DMSO was added 1:100 to the cells and incubated for 1 hour. The NLRP3 inflammasome was activated with the addition of 15 μM (final concentration) Nigericin (Enzo Life Sciences, #BML-CA421-0005), and cells were incubated for 3 hours. 10 μL supernatant was removed, and IL-1β levels were monitored using an HTRF assay (CisBio, #62IL1PEC) according to manufacturers' instructions. Viability and pyroptosis was monitored with the addition of PrestoBlue cell viability reagent (Life Technologies, #A13261) directly to the cell culture plate.

Further, enumerated, embodiments of the invention are defined below:
101. A compound of Formula AA

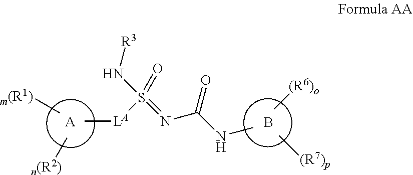

wherein

L^{A} is a bond or a C₁-C₈ alkylene optionally substituted with halo;

m=0, 1, or 2;

n=0, 1, or 2;

o=1 or 2;

p=0, 1, 2, or 3,

wherein

A is a 5- to 10-membered monocyclic or bicyclic heteroaryl or a C₆-C₁₀ monocyclic or bicyclic aryl;

B is a 5-10 membered monocyclic or bicyclic heteroaryl, or a C₆-C₁₀ monocyclic or bicyclic aryl; wherein at least one R⁶ is ortho to the bond connecting the B ring to the NH(CO) group of Formula AA;

R¹ and R² are each independently selected from C₁-C₆ alkyl, C₁-C₆ haloalkyl, C₁-C₆ alkoxy, C₁-C₆ haloalkoxy, halo, CN, NO₂, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NR⁸R⁹, C(O)R¹³, CONR⁸R⁹, SF₅, SC₁-C₆ alkyl, S(O₂)C₁-C₆ alkyl, S(O)C₁-C₆ alkyl, S(O₂)NR¹¹R¹², C₃-C₇ cycloalkyl, and 3- to 7-membered heterocycloalkyl,

wherein the C₁-C₆ alkyl, C₁-C₆ alkoxy, C₁-C₆ haloalkyl, C₆-C₁₀ aryl, 5- to 10-membered heteroaryl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, R¹⁵, C₁-C₆ haloalkoxy, NR⁸R⁹, ═NR¹⁰, COOC₁-C₆ alkyl, CONR⁸R⁹, 3- to 7-membered heterocycloalkyl, C₃-C₇ cycloalkyl, C₆-C₁₀ aryl, 5- to 10-membered heteroaryl, OCOC₁-C₆ alkyl, OCOC₆-C₁₀ aryl, OCO(5- to 10-membered heteroaryl), and OCO(3- to 7-membered heterocycloalkyl);

wherein each C₁-C₆ alkyl substituent and each C₁-C₆ alkoxy substituent of the R¹ or R² C₃-C₇ cycloalkyl or of the R¹ or R² 3- to 7-membered heterocycloalkyl is further optionally independently substituted with one to three hydroxy, —O(C₀-C₃ alkylene)C₆-C₁₀ aryl, halo, NR⁸R⁹, or oxo;

wherein the 3- to 7-membered heterocycloalkyl, C₆-C₁₀ aryl, and 5- to 10-membered heteroaryl are optionally substituted with one or more substituents independently selected from halo, C₁-C₆ alkyl, and OC₁-C₆ alkyl;

or one pair of R¹ and R² on adjacent atoms, taken together with the atoms connecting them, independently form one monocyclic or bicyclic C₄-C₁₂ carbocyclic ring or one monocyclic or bicyclic 5- to-12-membered heterocyclic ring containing 1-3 heteroatoms independently selected from O, N, and S, wherein the carbocyclic ring or heterocyclic ring is optionally independently substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR⁸R⁹, ═NR¹⁰, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR⁸R⁹,

wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR⁸R⁹, ═NR¹⁰, COOC₁-C₆ alkyl, C₆-C₁₀ aryl, and CONR⁸R⁹;

R⁶ and R⁷ are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, C₃-C₁₀ cycloalkyl, 3- to 10-membered heterocycloalkyl, and C₂-C₆ alkenyl,

wherein R⁶ and R⁷ are each optionally substituted with one or more substituents independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), C₆-C₁₀ aryloxy, and S(O₂)C₁-C₆ alkyl; and wherein the C₁-C₆ alkyl or C₁-C₆ alkoxy that R⁶ or R⁷ is substituted with is optionally substituted with one or more hydroxyl, C₆-C₁₀ aryl or NR⁸R⁹, or wherein R⁶ or R⁷ is optionally fused to a five- to -seven-membered carbocyclic ring or heterocyclic ring containing one or two heteroatoms independently selected from oxygen, sulfur and nitrogen;

wherein the 3- to 7-membered heterocycloalkyl, C₆-C₁₀ aryl, 5- to 10-membered heteroaryl are optionally substituted with one or more substituents independently selected from halo, C₁-C₆ alkyl, and OC₁-C₆ alkyl;

or at least one pair of R⁶ and R⁷ on adjacent atoms, taken together with the atoms connecting them, independently form at least one C₄-C₈ carbocyclic ring or at least one 5- to 8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹;

R¹⁰ is C₁-C₆ alkyl;

each of R⁸ and R⁹ at each occurrence is independently selected from hydrogen, C₁-C₆ alkyl, C₂-C₆ alkenyl, C₃-C₇ cycloalkyl, (C═NR¹³)NR¹¹R¹², S(O₂)C₁-C₆ alkyl, S(O₂)NR¹¹R¹², COR¹³, CO₂R¹³ and CONR¹¹R¹²; wherein the C₁-C₆ alkyl is optionally substituted with one or more hydroxy, halo, C₁-C₆ alkoxy, C₆-C₁₀ aryl, 5- to 10-membered heteroaryl, C₃-C₇ cycloalkyl, 3- to 7-membered heterocycloalkyl, or NR¹¹R¹²;

or R⁸ and R⁹ taken together with the nitrogen they are attached to form a 3- to 10-membered monocyclic or bicyclic ring optionally containing one or more heteroatoms in addition to the nitrogen they are attached to, wherein the ring is optionally substituted with one or more substituents independently selected from halo, C₁-C₆ alkyl, C₁-C₆ haloalkyl, C₁-C₆ alkoxy, oxo, N(C₁-C₆alkyl)₂, NH₂, NH(C₁-C₆ alkyl), and hydroxy;

R¹³ is C₁-C₆ alkyl, C₁-C₆ haloalkyl, or —(Z¹-Z²)ₐ₁-Z³;

each of R¹¹ and R¹² at each occurrence is independently selected from hydrogen, C₁-C₆ alkyl, and —(Z¹-Z²)ₐ₁-Z³;

a1 is 0-10 (e.g., 0-4);

each Z¹ is independently C₁-C₆ alkylene optionally substituted with one or more substituents independently selected from oxo, halo, and hydroxy;

each Z² is independently a bond, NH, N(C₁-C₆ alkyl), —O—, —S—, or 5-10 membered heteroarylene;

Z³ is independently C₆-C₁₀ aryl, C₂-C₆ alkenyl, C₂-C₆ alkynyl, C₃-C₁₀ cycloalkyl, 5- to 10-membered heteroaryl, or 3- to 10-membered heterocycloalkyl, each of which is optionally substituted with one or more substituents independently selected from halo, C₁-C₆ alkyl, C₁₋₆ haloalkyl, C₁-C₆ alkoxy, oxo, N(C₁-C₆alkyl)₂, NH₂, NH(C₁-C₆ alkyl), and hydroxy;

R³ is selected from cyano, hydroxy, C₁-C₆ alkoxy, C(O)OR′, C(O)OR¹⁴, C(O)R′, C(O)NR′R″, C(O)NHR¹⁴, S(O)₀₋₂R′, S(O)₀₋₂R¹⁴, S(O)₁₋₂NR′R″, S(O)₁₋₂NHR¹⁴, and R¹⁴—(C₀-C₂ alkylene)-, wherein the C₀-C₂ alkylene group is optionally substituted with one or more oxo;

R¹⁴ is C₃-C₅ cycloalkyl, 3- to 8-membered heterocycloalkyl, C₂-C₁₀ alkenyl, C₂-C₁₀ alkynyl, 5- to 10-membered monocyclic or bicyclic heteroaryl or C₆-C₁₀ monocyclic or bicyclic aryl, wherein each cycloalkyl, heterocycloalkyl, C₂-C₁₀ alkenyl, C₂-C₁₀ alkynyl, aryl or heteroaryl is optionally independently substituted with 1 or 2 R⁶;

each of R′ and R″ is independently hydrogen or C₁-C₆ alkyl;

R¹⁵ is —(Z⁴-Z⁵)ₐ₂-Z⁶;

a2 is an integer selected from 1-10 (e.g., 1-5);

each Z⁴ is independently selected from —O—, —S—, —NH—, and —N(C₁-C₃ alkyl)-;

provided that the Z⁴ group directly attached to R¹ or R² is —O— or —S—;

each Z⁵ is independently C₁-C₆ alkylene optionally substituted with one or more substituents independently selected from oxo, halo, and hydroxy; and

Z⁶ is OH, OC₁-C₆ alkyl, NH₂, NH(C₁-C₆ alkyl), N(C₁-C₆ alkyl)₂, NHC(O)(C₁-C₆ alkyl), NHC(O)(C₁-C₆ alkoxy), or an optionally substituted group selected from the group consisting of:

C₆-C₁₀ aryl, C₂-C₆ alkenyl, C₂-C₆ alkynyl, C₃-C₁₀ cycloalkyl, 5- to 10-membered heteroaryl, or 3- to 10-membered heterocycloalkyl, each of which is optionally substituted with one or more substituents independently selected from halo, C₁-C₆ alkyl, C₁₋₆ haloalkyl, C₁-C₆ alkoxy, oxo, N(C₁-C₆alkyl)₂, NH₂, NH(C₁-C₆ alkyl), and hydroxy;

or a pharmaceutically acceptable salt thereof;

provided that the compound is other than one or more compounds disclosed in WO 2018225018, which is incorporated herein by reference in its entirety; and

the compound is other than one or more compounds disclosed in IN 201721020305, which is incorporated herein by reference in its entirety.
2. A compound of Formula AA

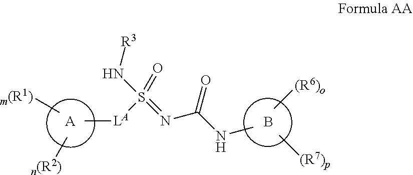

of embodiment 1 wherein:
the

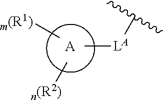

the moiety is as defined according to (AA-1), (AA-2), (AA-3), or (AA-4) below:
(AA-1)

L^{A} is a bond;

m=0, 1, or 2;

n=0, 1, or 2, wherein m+n >2; and

A is a 5- to 10-membered monocyclic or bicyclic heteroaryl wherein one ring atom is a heteroatom selected from O, N, and S; OR
(AA-2)

L^{A} is a bond;

m=0, 1, or 2;

n=0, 1, or 2; and

A is a 5- to 10-membered monocyclic or bicyclic heteroaryl wherein from 2-3 ring atoms are heteroatoms, each independently selected from O, N, and S; or a C₇-C₁₀ bicyclic aryl; OR
(AA-3)

L^{A} is a C₁-C₈ alkylene optionally substituted with halo;

m=0, 1, or 2;

n=0, 1, or 2; and

A is a 5- to 10-membered monocyclic or bicyclic heteroaryl wherein one ring atom is a heteroatom selected from O, N, and S; or a C₇-C₁₀ bicyclic aryl;
(AA-4)

L^{A} is a C₁-C₈ alkylene optionally substituted with halo;

m=0, 1, or 2; and

n=0, 1, or 2; wherein m+n>2;

A is phenyl;

B is a 5-10 membered monocyclic or bicyclic heteroaryl, or a C₆-C₁₀ monocyclic or bicyclic aryl; wherein at least one R⁶ is ortho to the bond connecting the B ring to the -L-NH(CO) group of Formula AA;

wherein when (AA-1), (AA-2), (AA-3), or (AA-4) applies:

R¹ and R² are each independently selected from C₁-C₆ alkyl, C₁-C₆ haloalkyl, C₁-C₆ alkoxy, C₁-C₆ haloalkoxy, halo, CN, NO₂, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NR⁸R⁹, C(O)R¹³, CONR⁸R⁹, SF₅, SC₁-C₆ alkyl, S(O₂)C₁-C₆ alkyl, S(O)C₁-C₆ alkyl, S(O₂)NR¹¹R¹², C₃-C₇ cycloalkyl, and 3- to 7-membered heterocycloalkyl,

wherein the C₁-C₆ alkyl, C₁-C₆ alkoxy, C₁-C₆ haloalkyl, C₆-C₁₀ aryl, 5- to 10-membered heteroaryl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, R¹⁵, C₁-C₆ haloalkoxy, NR⁸R⁹, ═NR¹⁰, COOC₁-C₆ alkyl, CONR⁸R⁹, 3- to 7-membered heterocycloalkyl, C₃-C₇ cycloalkyl, C₆-C₁₀ aryl, 5- to 10-membered heteroaryl, OCOC₁-C₆ alkyl, OCOC₆-C₁₀ aryl, OCO(5- to 10-membered heteroaryl), and OCO(3- to 7-membered heterocycloalkyl);

wherein each C₁-C₆ alkyl substituent and each C₁-C₆ alkoxy substituent of the R¹ or R² C₃-C₇ cycloalkyl or of the R¹ or R² 3- to 7-membered heterocycloalkyl is further optionally independently substituted with one to three hydroxy, —O(C₀-C₃ alkylene)C₆-C₁₀ aryl, halo, NR⁸R⁹, or oxo;

wherein the 3- to 7-membered heterocycloalkyl, C₆-C₁₀ aryl, and 5- to 10-membered heteroaryl are optionally substituted with one or more substituents independently selected from halo, C₁-C₆ alkyl, and OC₁-C₆ alkyl;

or one pair of R¹ and R² on adjacent atoms, taken together with the atoms connecting them, independently form one monocyclic or bicyclic C₄-C₁₂ carbocyclic ring or one monocyclic or bicyclic 5- to-12-membered heterocyclic ring containing 1-3 heteroatoms independently selected from O, N, and S, wherein the carbocyclic ring or heterocyclic ring is optionally independently substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR⁸R⁹, ═NR¹⁰, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR⁸R⁹,

wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR⁸R⁹, ═NR¹⁰, COOC₁-C₆ alkyl, C₆-C₁₀ aryl, and CONR⁸R⁹;

R⁶ and R⁷ are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO₂C₁-C₆ alkyl, CO₂C₃-C₅ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, C₃-C₁₀ cycloalkyl, 3- to 10-membered heterocycloalkyl, and C₂-C₆ alkenyl,

wherein R⁶ and R⁷ are each optionally substituted with one or more substituents independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), C₆-C₁₀ aryloxy, and S(O₂)C₁-C₆ alkyl; and wherein the C₁-C₆ alkyl or C₁-C₆ alkoxy that R⁶ or R⁷ is substituted with is optionally substituted with one or more hydroxyl, C₆-C₁₀ aryl or NR⁸R⁹, or wherein R⁶ or R⁷ is optionally fused to a five- to -seven-membered carbocyclic ring or heterocyclic ring containing one or two heteroatoms independently selected from oxygen, sulfur and nitrogen;

wherein the 3- to 7-membered heterocycloalkyl, C₆-C₁₀ aryl, 5- to 10-membered heteroaryl are optionally substituted with one or more substituents independently selected from halo, C₁-C₆ alkyl, and OC₁-C₆ alkyl;

or at least one pair of R⁶ and R⁷ on adjacent atoms, taken together with the atoms connecting them, independently form at least one C₄-C₈ carbocyclic ring or at least one 5- to 8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹;

R¹⁰ is C₁-C₆ alkyl;

each of R⁸ and R⁹ at each occurrence is independently selected from hydrogen, C₁-C₆ alkyl, C₂-C₆ alkenyl, C₃-C₇ cycloalkyl, (C═NR¹³)NR¹¹R¹², S(O₂)C₁-C₆ alkyl, S(O₂)NR¹¹R¹², COR¹³, CO₂R¹³ and CONR¹¹R¹²; wherein the C₁-C₆ alkyl is optionally substituted with one or more hydroxy, halo, C₁-C₆ alkoxy, C₆-C₁₀ aryl, 5- to 10-membered heteroaryl, C₃-C₇ cycloalkyl, 3- to 7-membered heterocycloalkyl, or NR¹¹R¹²;

or R⁸ and R⁹ taken together with the nitrogen they are attached to form a 3- to 10-membered monocyclic or bicyclic ring optionally containing one or more heteroatoms in addition to the nitrogen they are attached to, wherein the ring is optionally substituted with one or more substituents independently selected from halo, C₁-C₆ alkyl, C₁-C₆ haloalkyl, C₁-C₆ alkoxy, oxo, N(C₁-C₆alkyl)₂, NH₂, NH(C₁-C₆ alkyl), and hydroxy;

R¹³ is C₁-C₆ alkyl, C₁-C₆ haloalkyl, or —(Z¹-Z²)ₐ₁-Z³;

each of R¹¹ and R¹² at each occurrence is independently selected from hydrogen, C₁-C₆ alkyl, and —(Z¹-Z²)ₐ₁-Z³;

a1 is 0-10 (e.g., 0-4);

each Z¹ is independently C₁-C₆ alkylene optionally substituted with one or more substituents independently selected from oxo, halo, and hydroxy;

each Z² is independently a bond, NH, N(C₁-C₆ alkyl), —O—, —S—, or 5-10 membered heteroarylene;

Z³ is independently C₆-C₁₀ aryl, C₂-C₆ alkenyl, C₂-C₆ alkynyl, C₃-C₁₀ cycloalkyl, 5- to 10-membered heteroaryl, or 3- to 10-membered heterocycloalkyl, each of which is optionally substituted with one or more substituents independently selected from halo, C₁-C₆ alkyl, C₁₋₆ haloalkyl, C₁-C₆ alkoxy, oxo, N(C₁-C₆alkyl)₂, NH₂, NH(C₁-C₆ alkyl), and hydroxy;

R³ is selected from cyano, hydroxy, C₁-C₆ alkoxy, C(O)OR′, C(O)OR¹⁴, C(O)R′, C(O)NR′R″, C(O)NHR¹⁴, S(O)₀₋₂R′, S(O)₀₋₂R¹⁴, S(O)₁₋₂NR′R″, S(O)₁₋₂NHR¹⁴, and R¹⁴—(C₀-C₂ alkylene)-, wherein the C₀-C₂ alkylene group is optionally substituted with one or more oxo;

R¹⁴ is C₃-C₅ cycloalkyl, 3- to 8-membered heterocycloalkyl, C₂-C₁₀ alkenyl, C₂-C₁₀ alkynyl, 5- to 10-membered monocyclic or bicyclic heteroaryl or C₆-C₁₀ monocyclic or bicyclic aryl, wherein each cycloalkyl, heterocycloalkyl, C₂-C₁₀ alkenyl, C₂-C₁₀ alkynyl, aryl or heteroaryl is optionally independently substituted with 1 or 2 R⁶;

each of R′ and R″ is independently hydrogen or C₁-C₆ alkyl;

R¹⁵ is —(Z⁴-Z⁵)ₐ₂-Z⁶;

a2 is an integer selected from 1-10 (e.g., 1-5);

each Z⁴ is independently selected from —O—, —S—, —NH—, and —N(C₁-C₃ alkyl)-;

provided that the Z⁴ group directly attached to R¹ or R² is —O— or —S—;

each Z⁵ is independently C₁-C₆ alkylene optionally substituted with one or more substituents independently selected from oxo, halo, and hydroxy; and

Z⁶ is OH, OC₁-C₆ alkyl, NH₂, NH(C₁-C₆ alkyl), N(C₁-C₆ alkyl)₂, NHC(O)(C₁-C₆ alkyl), NHC(O)(C₁-C₆ alkoxy), or an optionally substituted group selected from the group consisting of:

C₆-C₁₀ aryl, C₂-C₆ alkenyl, C₂-C₆ alkynyl, C₃-C₁₀ cycloalkyl, 5- to 10-membered heteroaryl, or 3- to 10-membered heterocycloalkyl, each of which is optionally substituted with one or more substituents independently selected from halo, C₁-C₆ alkyl, C₁₋₆ haloalkyl, C₁-C₆ alkoxy, oxo, N(C₁-C₆ alkyl)₂, NH₂, NH(C₁-C₆ alkyl), and hydroxy;

or a pharmaceutically acceptable salt thereof.
3. A compound of Formula AA

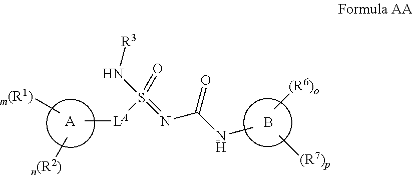

of embodiment 1 wherein

L^{A} is a bond or a C₁-C₈ alkylene optionally substituted with halo;

m=0, 1, or 2;

n=0, 1, or 2;

wherein

A is a 5- to 10-membered monocyclic or bicyclic heteroaryl or a C₆-C₁₀ monocyclic or bicyclic aryl;

R¹ and R² are each independently selected from C₁-C₆ alkyl, C₁-C₆ haloalkyl, C₁-C₆ alkoxy, C₁-C₆ haloalkoxy, halo, CN, NO₂, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NR⁸R⁹, C(O)R¹³, CONR⁸R⁹, SF₅, SC₁-C₆ alkyl, S(O₂)C₁-C₆ alkyl, S(O)C₁-C₆ alkyl, S(O₂)NR¹¹R¹², C₃-C₇ cycloalkyl, and 3- to 7-membered heterocycloalkyl,

wherein the C₁-C₆ alkyl, C₁-C₆ alkoxy, C₁-C₆ haloalkyl, C₆-C₁₀ aryl, 5- to 10-membered heteroaryl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, R¹⁵, C₁-C₆ haloalkoxy, NR⁸R⁹, ═NR¹⁰, COOC₁-C₆ alkyl, CONR⁸R⁹, 3- to 7-membered heterocycloalkyl, C₃-C₇ cycloalkyl, C₆-C₁₀ aryl, 5- to 10-membered heteroaryl, OCOC₁-C₆ alkyl, OCOC₆-C₁₀ aryl, OCO(5- to 10-membered heteroaryl), and OCO(3- to 7-membered heterocycloalkyl);

wherein each C₁-C₆ alkyl substituent and each C₁-C₆ alkoxy substituent of the R¹ or R² C₃-C₇ cycloalkyl or of the R¹ or R² 3- to 7-membered heterocycloalkyl is further optionally independently substituted with one to three hydroxy, —O(C₀-C₃ alkylene)C₆-C₁₀ aryl, halo, NR⁸R⁹, or oxo;

wherein the 3- to 7-membered heterocycloalkyl, C₆-C₁₀ aryl, and 5- to 10-membered heteroaryl are optionally substituted with one or more substituents independently selected from halo, C₁-C₆ alkyl, and OC₁-C₆ alkyl;

or one pair of R¹ and R² on adjacent atoms, taken together with the atoms connecting them, independently form one monocyclic or bicyclic C₄-C₁₂ carbocyclic ring or one monocyclic or bicyclic 5- to-12-membered heterocyclic ring containing 1-3 heteroatoms independently selected from O, N, and S, wherein the carbocyclic ring or heterocyclic ring is optionally independently substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR⁸R⁹, ═NR¹⁰, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR⁸R⁹,

wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR⁸R⁹, ═NR¹⁰, COOC₁-C₆ alkyl, C₆-C₁₀ aryl, and CONR⁸R⁹;

wherein the

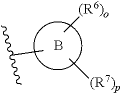

moiety is as defined in (BB-1) or (BB-2) below:

(BB-1)

o=1 or 2;

p=0, 1, 2, or 3,

B is a 5-10 membered monocyclic or bicyclic heteroaryl; wherein at least one R⁶ is ortho to the bond connecting the B ring to the NH(CO) group of Formula AA;

(BB-2)

o=1 or 2;

p=0, 1, 2, or 3,

provided that o+p>2;

B is a C₆-C₁₀ monocyclic or bicyclic aryl; wherein at least one R⁶ is ortho to the bond connecting the B ring to the -L-NH(CO) group of Formula AA;

when (BB-1) or (BB-2) applies:

R⁶ and R⁷ are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, C₃-C₁₀ cycloalkyl, 3- to 10-membered heterocycloalkyl, and C₂-C₆ alkenyl,

wherein R⁶ and R⁷ are each optionally substituted with one or more substituents independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), C₆-C₁₀ aryloxy, and S(O₂)C₁-C₆ alkyl; and wherein the C₁-C₆ alkyl or C₁-C₆ alkoxy that R⁶ or R⁷ is substituted with is optionally substituted with one or more hydroxyl, C₆-C₁₀ aryl or NR⁸R⁹, or wherein R⁶ or R⁷ is optionally fused to a five- to -seven-membered carbocyclic ring or heterocyclic ring containing one or two heteroatoms independently selected from oxygen, sulfur and nitrogen;

wherein the 3- to 7-membered heterocycloalkyl, C₆-C₁₀ aryl, 5- to 10-membered heteroaryl are optionally substituted with one or more substituents independently selected from halo, C₁-C₆ alkyl, and OC₁-C₆ alkyl;

or at least one pair of R⁶ and Won adjacent atoms, taken together with the atoms connecting them, independently form at least one C₄-C₈ carbocyclic ring or at least one 5- to 8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹; OR

provided that:

when (BB-2) applies; o+p=4; two pairs of R⁶ and R⁷ are on adjacent atoms; and

each pair taken together with the atoms connecting them, independently forms a ring selected from a C₄-C₈ carbocyclic ring and a 5- to 8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹,

then at least one ring is selected from:

(a) a C₄ carbocyclic ring, a C₆-C₈ carbocyclic ring, or a 5- to 8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹; and

(b) a C₅ carbocyclic ring substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹;

R¹⁰ is C₁-C₆ alkyl;

each of R⁸ and R⁹ at each occurrence is independently selected from hydrogen, C₁-C₆ alkyl, C₂-C₆ alkenyl, C₃-C₇ cycloalkyl, (C═NR¹³)NR¹¹R¹², S(O₂)C₁-C₆ alkyl, S(O₂)NR¹¹R¹², COR¹³, CO₂R¹³ and CONR¹¹R¹²; wherein the C₁-C₆ alkyl is optionally substituted with one or more hydroxy, halo, C₁-C₆ alkoxy, C₆-C₁₀ aryl, 5- to 10-membered heteroaryl, C₃-C₇ cycloalkyl, 3- to 7-membered heterocycloalkyl, or NR¹¹R¹²;

or R⁸ and R⁹ taken together with the nitrogen they are attached to form a 3- to 10-membered monocyclic or bicyclic ring optionally containing one or more heteroatoms in addition to the nitrogen they are attached to, wherein the ring is optionally substituted with one or more substituents independently selected from halo, C₁-C₆ alkyl, C₁-C₆ haloalkyl, C₁-C₆ alkoxy, oxo, N(C₁-C₆alkyl)₂, NH₂, NH(C₁-C₆ alkyl), and hydroxy;

R¹³ is C₁-C₆ alkyl, C₁-C₆ haloalkyl, or —(Z¹-Z²)ₐ₁-Z³;

each of R¹¹ and R¹² at each occurrence is independently selected from hydrogen, C₁-C₆ alkyl, and —(Z¹-Z²)ₐ₁-Z³;

a1 is 0-10 (e.g., 0-4);

each Z¹ is independently C₁-C₆ alkylene optionally substituted with one or more substituents independently selected from oxo, halo, and hydroxy;

each Z² is independently a bond, NH, N(C₁-C₆ alkyl), —O—, —S—, or 5-10 membered heteroarylene;

Z³ is independently C₆-C₁₀ aryl, C₂-C₆ alkenyl, C₂-C₆ alkynyl, C₃-C₁₀ cycloalkyl, 5- to 10-membered heteroaryl, or 3- to 10-membered heterocycloalkyl, each of which is optionally substituted with one or more substituents independently selected from halo, C₁-C₆ alkyl, C₁₋₆ haloalkyl, C₁-C₆ alkoxy, oxo, N(C₁-C₆ alkyl)₂, NH₂, NH(C₁-C₆ alkyl), and hydroxy;

R³ is selected from cyano, hydroxy, C₁-C₆ alkoxy, C(O)OR′, C(O)OR¹⁴, C(O)R′, C(O)NR′R″, C(O)NHR¹⁴, S(O)₀₋₂R′, S(O)₀₋₂R¹⁴, S(O)₁₋₂NR′R″, S(O)₁₋₂NHR¹⁴, and R¹⁴—(C₀-C₂ alkylene)-, wherein the C₀-C₂ alkylene group is optionally substituted with one or more oxo;

R¹⁴ is C₃-C₈ cycloalkyl, 3- to 8-membered heterocycloalkyl, C₂-C₁₀ alkenyl, C₂-C₁₀ alkynyl, 5- to 10-membered monocyclic or bicyclic heteroaryl or C₆-C₁₀ monocyclic or bicyclic aryl, wherein each cycloalkyl, heterocycloalkyl, C₂-C₁₀ alkenyl, C₂-C₁₀ alkynyl, aryl or heteroaryl is optionally independently substituted with 1 or 2 R⁶;

each of R′ and R″ is independently hydrogen or C₁-C₆ alkyl;

R¹⁵ is —(Z⁴-Z⁵)ₐ₂-Z⁶;

a2 is an integer selected from 1-10 (e.g., 1-5);

each Z⁴ is independently selected from —O—, —S—, —NH—, and —N(C₁-C₃ alkyl)-;

provided that the Z⁴ group directly attached to R¹ or R² is —O— or —S—;

each Z⁵ is independently C₁-C₆ alkylene optionally substituted with one or more substituents independently selected from oxo, halo, and hydroxy; and

Z⁶ is OH, OC₁-C₆ alkyl, NH₂, NH(C₁-C₆ alkyl), N(C₁-C₆ alkyl)₂, NHC(O)(C₁-C₆ alkyl), NHC(O)(C₁-C₆ alkoxy), or an optionally substituted group selected from the group consisting of:

C₆-C₁₀ aryl, C₂-C₆ alkenyl, C₂-C₆ alkynyl, C₃-C₁₀ cycloalkyl, 5- to 10-membered heteroaryl, or 3- to 10-membered heterocycloalkyl, each of which is optionally substituted with one or more substituents independently selected from halo, C₁-C₆ alkyl, C₁₋₆ haloalkyl, C₁-C₆ alkoxy, oxo, N(C₁-C₆ alkyl)₂, NH₂, NH(C₁-C₆ alkyl), and hydroxy;

or a pharmaceutically acceptable salt thereof.
4. A compound of Formula AA:

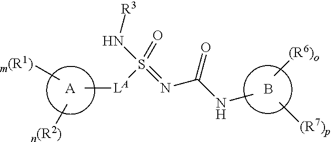

of embodiment 1 wherein the compound is an unequal mixture (e.g., a non-racemic mixture when the sulfur is the only stereocenter in Formula AA) of:

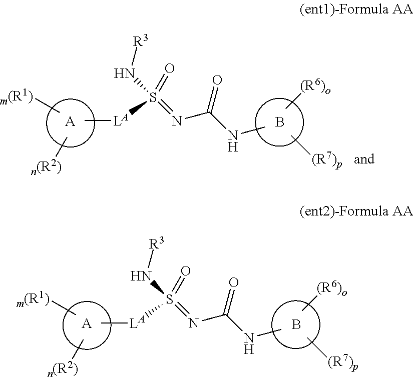

wherein

L^{A} is a bond or a C₁-C₈ alkylene optionally substituted with halo;

m=0, 1, or 2;

n=0, 1, or 2;

o=1 or 2;

p=0, 1, 2, or 3,

wherein

A is a 5- to 10-membered monocyclic or bicyclic heteroaryl or a C₆-C₁₀ monocyclic or bicyclic aryl;

B is a 5-10 membered monocyclic or bicyclic heteroaryl, or a C₆-C₁₀ monocyclic or bicyclic aryl; wherein at least one R⁶ is ortho to the bond connecting the B ring to the NH(CO) group of Formula AA;

R¹ and R² are each independently selected from C₁-C₆ alkyl, C₁-C₆ haloalkyl, C₁-C₆ alkoxy, C₁-C₆ haloalkoxy, halo, CN, NO₂, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NR⁸R⁹, C(O)R¹³, CONR⁸R⁹, SF₅, SC₁-C₆ alkyl, S(O₂)C₁-C₆ alkyl, S(O)C₁-C₆ alkyl, S(O₂)NR¹¹R¹², C₃-C₇ cycloalkyl, and 3- to 7-membered heterocycloalkyl,

wherein the C₁-C₆ alkyl, C₁-C₆ alkoxy, C₁-C₆ haloalkyl, C₆-C₁₀ aryl, 5- to 10-membered heteroaryl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, R¹⁵, C₁-C₆ haloalkoxy, NR⁸R⁹, ═NR¹⁰, COOC₁-C₆ alkyl, CONR⁸R⁹, 3- to 7-membered heterocycloalkyl, C₃-C₇ cycloalkyl, C₆-C₁₀ aryl, 5- to 10-membered heteroaryl, OCOC₁-C₆ alkyl, OCOC₆-C₁₀ aryl, OCO(5- to 10-membered heteroaryl), and OCO(3- to 7-membered heterocycloalkyl);

wherein each C₁-C₆ alkyl substituent and each C₁-C₆ alkoxy substituent of the R¹ or R² C₃-C₇ cycloalkyl or of the R¹ or R² 3- to 7-membered heterocycloalkyl is further optionally independently substituted with one to three hydroxy, —O(C₀-C₃ alkylene)C₆-C₁₀ aryl, halo, NR⁸R⁹, or oxo;

wherein the 3- to 7-membered heterocycloalkyl, C₆-C₁₀ aryl, and 5- to 10-membered heteroaryl are optionally substituted with one or more substituents independently selected from halo, C₁-C₆ alkyl, and OC₁-C₆ alkyl;

or one pair of R¹ and R² on adjacent atoms, taken together with the atoms connecting them, independently form one monocyclic or bicyclic C₄-C₁₂ carbocyclic ring or one monocyclic or bicyclic 5- to-12-membered heterocyclic ring containing 1-3 heteroatoms independently selected from O, N, and S, wherein the carbocyclic ring or heterocyclic ring is optionally independently substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR⁸R⁹, ═NR¹⁰, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR⁸R⁹,

wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR⁸R⁹, ═NR¹⁰, COOC₁-C₆ alkyl, C₆-C₁₀ aryl, and CONR⁸R⁹;

R⁶ and R⁷ are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, C₃-C₁₀ cycloalkyl, 3- to 10-membered heterocycloalkyl, and C₂-C₆ alkenyl,

wherein R⁶ and R⁷ are each optionally substituted with one or more substituents independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), C₆-C₁₀ aryloxy, and S(O₂)C₁-C₆ alkyl; and wherein the C₁-C₆ alkyl or C₁-C₆ alkoxy that R⁶ or R⁷ is substituted with is optionally substituted with one or more hydroxyl, C₆-C₁₀ aryl or NR⁸R⁹, or wherein R⁶ or R⁷ is optionally fused to a five- to -seven-membered carbocyclic ring or heterocyclic ring containing one or two heteroatoms independently selected from oxygen, sulfur and nitrogen;

wherein the 3- to 7-membered heterocycloalkyl, C₆-C₁₀ aryl, 5- to 10-membered heteroaryl are optionally substituted with one or more substituents independently selected from halo, C₁-C₆ alkyl, and OC₁-C₆ alkyl;

or at least one pair of R⁶ and R⁷ on adjacent atoms, taken together with the atoms connecting them, independently form at least one C₄-C₈ carbocyclic ring or at least one 5- to 8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹;

R¹⁰ is C₁-C₆ alkyl;

each of R⁸ and R⁹ at each occurrence is independently selected from hydrogen, C₁-C₆ alkyl, C₂-C₆ alkenyl, C₃-C₇ cycloalkyl, (C═NR¹³)NR¹¹R¹², S(O₂)C₁-C₆ alkyl, S(O₂)NR¹¹R¹², COR¹³, CO₂R¹³ and CONR¹¹R¹²; wherein the C₁-C₆ alkyl is optionally substituted with one or more hydroxy, halo, C₁-C₆ alkoxy, C₆-C₁₀ aryl, 5- to 10-membered heteroaryl, C₃-C₇ cycloalkyl, 3- to 7-membered heterocycloalkyl, or NR¹¹R¹²;

or R⁸ and R⁹ taken together with the nitrogen they are attached to form a 3- to 10-membered monocyclic or bicyclic ring optionally containing one or more heteroatoms in addition to the nitrogen they are attached to, wherein the ring is optionally substituted with one or more substituents independently selected from halo, C₁-C₆ alkyl, C₁-C₆ haloalkyl, C₁-C₆ alkoxy, oxo, N(C₁-C₆ alkyl)₂, NH₂, NH(C₁-C₆ alkyl), and hydroxy;

R¹³ is C₁-C₆ alkyl, C₁-C₆ haloalkyl, or —(Z¹-Z²)ₐ₁-Z³;

each of R¹¹ and R¹² at each occurrence is independently selected from hydrogen, C₁-C₆ alkyl, and —(Z¹-Z²)ₐ₁-Z³;

a1 is 0-10 (e.g., 0-4);

each Z¹ is independently C₁-C₆ alkylene optionally substituted with one or more substituents independently selected from oxo, halo, and hydroxy;

each Z² is independently a bond, NH, N(C₁-C₆ alkyl), —O—, —S—, or 5-10 membered heteroarylene;

Z³ is independently C₆-C₁₀ aryl, C₂-C₆ alkenyl, C₂-C₆ alkynyl, C₃-C₁₀ cycloalkyl, 5- to 10-membered heteroaryl, or 3- to 10-membered heterocycloalkyl, each of which is optionally substituted with one or more substituents independently selected from halo, C₁-C₆ alkyl, C₁₋₆ haloalkyl, C₁-C₆ alkoxy, oxo, N(C₁-C₆alkyl)₂, NH₂, NH(C₁-C₆ alkyl), and hydroxy;

R³ is selected from cyano, hydroxy, C₁-C₆ alkoxy, C(O)OR′, C(O)OR¹⁴, C(O)R′, C(O)NR′R″, C(O)NHR¹⁴, S(O)₀₋₂R′, S(O)₀₋₂R¹⁴, S(O)₁₋₂NR′R″, S(O)₁₋₂NHR¹⁴, and R¹⁴—(C₀-C₂ alkylene)-, wherein the C₀-C₂ alkylene group is optionally substituted with one or more oxo;

R¹⁴ is C₃-C₅ cycloalkyl, 3- to 8-membered heterocycloalkyl, C₂-C₁₀ alkenyl, C₂-C₁₀ alkynyl, 5- to 10-membered monocyclic or bicyclic heteroaryl or C₆-C₁₀ monocyclic or bicyclic aryl, wherein each cycloalkyl, heterocycloalkyl, C₂-C₁₀ alkenyl, C₂-C₁₀ alkynyl, aryl or heteroaryl is optionally independently substituted with 1 or 2 R⁶;

each of R′ and R″ is independently hydrogen or C₁-C₆ alkyl;

R¹⁵ is —(Z⁴-Z⁵)ₐ₂-Z⁶;

a2 is an integer selected from 1-10 (e.g., 1-5);

each Z⁴ is independently selected from —O—, —S—, —NH—, and —N(C₁-C₃ alkyl)-;

provided that the Z⁴ group directly attached to R¹ or R² is —O— or —S—;

each Z⁵ is independently C₁-C₆ alkylene optionally substituted with one or more substituents independently selected from oxo, halo, and hydroxy; and

Z⁶ is OH, OC₁-C₆ alkyl, NH₂, NH(C₁-C₆ alkyl), N(C₁-C₆ alkyl)₂, NHC(O)(C₁-C₆ alkyl), NHC(O)(C₁-C₆ alkoxy), or an optionally substituted group selected from the group consisting of:

C₆-C₁₀ aryl, C₂-C₆ alkenyl, C₂-C₆ alkynyl, C₃-C₁₀ cycloalkyl, 5- to 10-membered heteroaryl, or 3- to 10-membered heterocycloalkyl, each of which is optionally substituted with one or more substituents independently selected from halo, C₁-C₆ alkyl, C₁₋₆ haloalkyl, C₁-C₆ alkoxy, oxo, N(C₁-C₆alkyl)₂, NH₂, NH(C₁-C₆ alkyl), and hydroxy;

or a pharmaceutically acceptable salt thereof.
5. The compound of embodiment 4, wherein the mixture is enriched in (ent1)-Formula AA.
6. The compound of embodiment 4, wherein the mixture is enriched in (ent2)-Formula AA.
7. The compound of any one of embodiments 4-5, wherein the compound is a compound of (ent1)-Formula AA that is substantially free of (ent2)-Formula AA.
8. The compound of any one of embodiments 4 and 6, wherein the compound is a compound of (ent2)-Formula AA that is substantially free of (ent1)-Formula AA.
9. The compound of any one of embodiments 1-8, wherein R³ is selected from the group consisting of: CN, C(O)R′, C(O)OR¹⁴, C(O)OR′, C(O)NR′R″, C(O)NHR¹⁴, S(O)₀₋₂R¹⁴, S(O)₀₋₂R′, S(O)₁₋₂NR′R″, S(O)₁₋₂NHR¹⁴, and C(O)R¹⁴ (e.g., C(O)R′ (e.g., C(O)H)).
10. The compound of any one of embodiments 1-9, wherein R³ is CN.
11. The compound of any one of embodiments 1-10, wherein A is a 5- to 6-membered monocyclic heteroaryl optionally substituted with 1 or 2 R¹ and optionally substituted with 1 or 2 R².
12. The compound of any one of embodiments 1-11, wherein A is furanyl optionally substituted with 1 or 2 R¹ and optionally substituted with 1 or 2 R².
13. The compound of any one of embodiments 1-11, wherein A is thiophenyl optionally substituted with 1 or 2 R¹ and optionally substituted with 1 or 2 R².
14. The compound of any one of embodiments 1-11, wherein A is oxazolyl optionally substituted with 1 or 2 R¹ and optionally substituted with 1 or 2 R².
15. The compound of any one of embodiments 1-11, wherein A is thiazolyl optionally substituted with 1 or 2 R¹ and optionally substituted with 1 or 2 R².
16. The compound of any one of embodiments 1-11, wherein A is pyrazolyl optionally substituted with 1 or 2 R¹ and optionally substituted with 1 or 2 R².
17. The compound of any one of embodiments 1-11, wherein A is imidazolyl optionally substituted with 1 or 2 R¹ and optionally substituted with 1 or 2 R².
18. The compound of any one of embodiments 1-11, wherein A is thiophenyl or furanyl optionally substituted with 1 or 2 R¹ and optionally substituted with 1 or 2 R², provided that m+n≥2.
19. The compound of any one of embodiments 1-10, wherein A is phenyl optionally substituted with 1 or 2 R¹ and optionally substituted with 1 or 2 R².
20. The compound of any one of embodiments 1-17, and 19, wherein m=1 and n=0.
21. The compound of any one of embodiments 1-11, 13, and 20, wherein A is

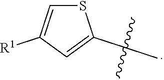

22. The compound of any one of embodiments 1-11, 13, and 20, wherein A is

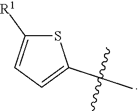

23. The compound of any one of embodiments 1-12, and 20, wherein A is

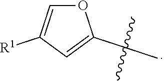

24. The compound of any one of embodiments 1-12, and 20, wherein A is

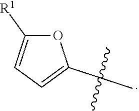

25. The compound of any one of embodiments 1-11, 15, and 20, wherein A is

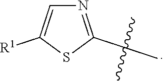

26. The compound of any one of embodiments 1-11, 15, and 20, wherein A is

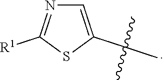

27. The compound of any one of embodiments 1-11, 15, and 20, wherein A is

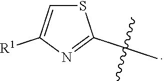

28. The compound of any one of embodiments 1, 3-10, and 19-20, wherein A is

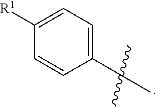

29. The compound of any one of embodiments 1, 3-10, and 19-20, wherein A is

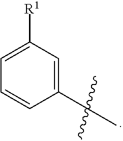

30. The compound of any one of embodiments 1, 3-10, and 19-20, wherein A is

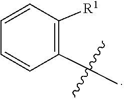

31. The compound of any one of embodiments 1 and 3-10, wherein A is

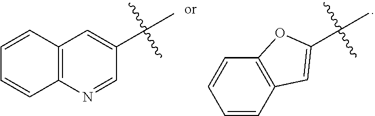 [SMILES: *c1cnc2ccccc2c1 ; *c1cc2ccccc2o1]

32. The compound of any one of embodiments 1-11, 16, and 20, wherein A is

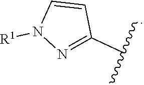

33. The compound of any one of embodiments 1-11, and 20, wherein A is

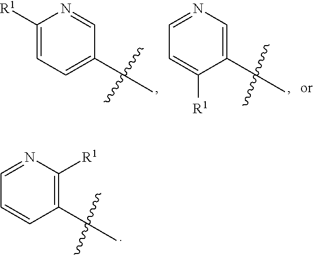

34. The compound of any one of embodiments 1-11, and 20, wherein A is

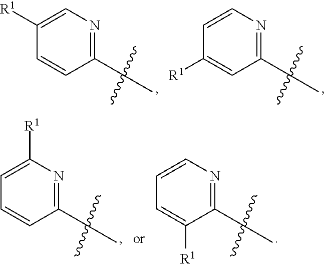

35. The compound of any one of embodiments 1-11, and 20, wherein A is

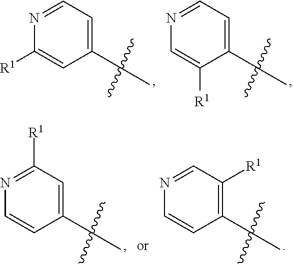

36. The compound of any one of embodiments 1-19, wherein m=1 and n=1.
37. The compound of any one of embodiments 1-11, 13, and 36, wherein A is

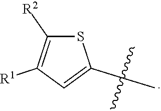

38. The compound of any one of embodiments 1-11, 15, and 36, wherein A is

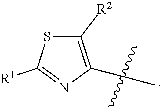

39. The compound of any one of embodiments 1-11, 15, and 36, wherein A is

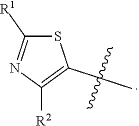

40. The compound of any one of embodiments 1-12 and 36, wherein A is

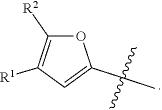

41. The compound of any one of embodiments 1-11, 13, and 36, wherein A is

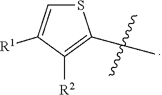

42. The compound of any one of embodiments 1-11, 13, and 36, wherein A is

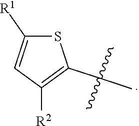

43. The compound of any one of embodiments 1-11, 15, and 36, wherein A is

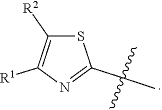

44. The compound of any one of embodiments 1-11, 16, and 36, wherein the optionally substituted ring A is

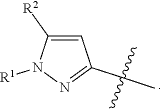

45. The compound of any one of embodiments 1-11, 16, and 36, wherein the optionally substituted ring A is

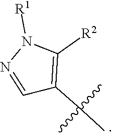

46. The compound of any one of embodiments 1-11, 16, and 36, wherein the optionally substituted ring A is

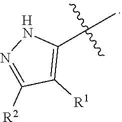

47. The compound of any one of embodiments 1-11, 17, and 36, wherein the optionally substituted ring A is

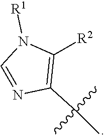

48. The compound of any one of embodiments 1-10, 19, and 36, wherein A is

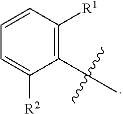

49. The compound of any one of embodiments 1-10, 19, and 36, wherein A is

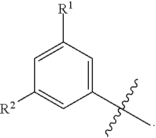

50. The compound of any one of embodiments 1-10, 19, and 36, wherein A is

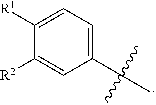

51. The compound of any one of embodiments 1-10, 19, and 36, wherein A is

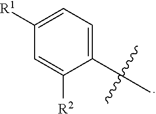

52. The compound of any one of embodiments 1-10, 19, and 36, wherein A is

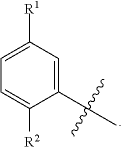

53. The compound of any one of embodiments 1-19, wherein m=2 and n=1.
54. The compound of any one of embodiments 1-11, 19 and 53, wherein A is

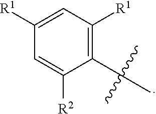

55. The compound of any one of embodiments 1-11, 19 and 53, wherein A is

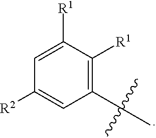

56. The compound of any one of embodiments 1-11, 19 and 53, wherein A is

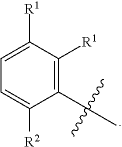

57. The compound of any one of embodiments 1-11, 19, and 53, wherein A is

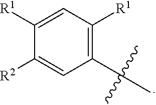

58. The compound of any one of embodiments 1-11, 16, and 53, wherein A is

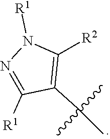

59. The compound of any one of embodiments 1-58, wherein each of R¹ and R², when present, is independently selected from the group consisting of C₁-C₆ alkyl optionally substituted with one or more hydroxy, halo, oxo, C₁-C₆ alkoxy, 3- to 7-membered heterocycloalkyl optionally substituted with halo, C₃-C₇ cycloalkyl, R¹⁵, C₁-C₆ haloalkoxy, C₆-C₁₀ aryl optionally substituted with OC₁-C₆ alkyl, or NR⁸R⁹; C₃-C₇ cycloalkyl optionally substituted with one or more hydroxy, halo, oxo, C₁-C₆ alkoxy, C₁-C₆ alkyl, or NR⁸R⁹ wherein the C₁-C₆ alkoxy or C₁-C₆ alkyl is further optionally substituted with one to three hydroxy, halo, NR⁸R⁹, or oxo; 3- to 7-membered heterocycloalkyl optionally substituted with one or more hydroxy, halo, oxo, C₁-C₆ alkyl, or NR⁸R⁹ wherein the C₁-C₆ alkoxy or C₁-C₆ alkyl is further optionally substituted with one to three hydroxy, halo, NR⁸R⁹, or oxo; C₁-C₆ haloalkyl; C₁-C₆ alkoxy; C₁-C₆ haloalkoxy; halo; CN; CO-C₁-C₆ alkyl; CO-C₆-C₁₀ aryl; CO(5- to 10-membered heteroaryl); CO₂C₁-C₆ alkyl; CO₂C₃-C₈ cycloalkyl; OCOC₁-C₆ alkyl; OCOC₆-C₁₀ aryl; OCO(5- to 10-membered heteroaryl); OCO(3- to 7-membered heterocycloalkyl); C₆-C₁₀ aryl; 5- to 10-membered heteroaryl; NH₂; NHC₁-C₆ alkyl; N(C₁-C₆ alkyl)₂; CONR⁸R⁹; SF₅; S(O₂)NR¹¹R¹²; S(O)C₁-C₆ alkyl; and S(O₂)C₁-C₆ alkyl.
60. The compound of any one of embodiments 1-58, wherein R¹ is selected from the group consisting of 1-hydroxy-2-methylpropan-2-yl; methyl; isopropyl; isobutyl; difluoromethyl; 2-hydroxy-2-propyl; hydroxymethyl; 1-hydroxyethyl; 2-hydroxyethyl; 1-hydroxy-2-propyl; 1,2-dihydroxy-2-propyl; 1,2,3-trihydroxy-2-propyl; 1-amino-2-hydroxy-prop-2-yl; 1-acetamido-2-hydroxy-prop-2-yl; 1-(tert-butoxycarbonyl)amino-2-hydroxy-prop-2-yl; 1-(2-hydroxyethoxy)-2-hydroxy-2-propyl; 1-(2-benzyloxyethoxy)-2-hydroxy-2-propyl; 1-(2-methoxyethoxy)-2-hydroxy-2-propyl; 1-hydroxy-1-cyclopropyl; 1-hydroxy-1-cyclobutyl; 1-hydroxy-1-cyclopentyl; 1-hydroxy-1-cyclohexyl; morpholinyl; pyrrolidinyl; 1,3-dioxolan-2-yl; COCH₃; COCH₂CH₃; difluoromethoxy; 2-methoxy-2-propyl; (methylamino)methyl; (2,2-difluoroeth-1-yl)(methyl)aminomethyl; (2,2,2-trifluoroeth-1-yl)(methyl)aminomethyl; (dimethylamino)methyl; 1-(dimethylamino)ethyl; 2-((methyl)aminomethyl)-prop-2-yl; 2-((methyl)amino)-prop-2-yl; (methyl)(cyclopropylmethyl)aminomethyl; (methyl)(2-(dimethylamino)eth-1-yl)aminomethyl; (cyclobutyl)(methyl)aminomethyl; 1-(cyclobutyl)amino-eth-1-yl; isopropylaminomethyl; (cyclobutyl)aminomethyl; cycloheptylaminomethyl; tetrahydropyranylaminomethyl; sec-butylaminomethyl; ethylaminomethyl; allylaminomethyl; (2,2-difluoroeth-1-yl)aminomethyl; (2-methoxy-eth-1-yl)aminomethyl; (2-methoxy-eth-1-yl)(methyl)aminomethyl; 2-fluoro-1-dimethylamino-eth-1-yl; 1-dimethylamino-2,2-difluoroeth-1-yl; 1-dimethylamino-2,2,2-trifluoroeth-1-yl; 1-dimethylamino-2,2,2-trimethyleth-1-yl; dimethylamino(cyclopropyl)methyl; methoxymethyl; isopropyl(methyl)amino; fluoro; chloro; phenyl; pyridyl; pyrazolyl; azetidinyl; 5-methyl-oxazolidin-2-one-5-yl; S(O₂)CH₃; S(O₂)NR¹¹R¹²; (3,3-difluoropyrrolidin-1-yl)methyl; 1-(difluoromethoxyl)eth-1-yl; azetidinylmethyl; 1-((methyl)aminomethyl)-cycloprop-1-yl; 4-methoxybenzyl; 4-methyl-piperazin-1-yl; morpholinylmethyl; and cyclopentyl.
61. The compound of any one of embodiments 59-60, wherein R² is selected from the group consisting of fluoro, chloro, cyano, methyl; methoxy; ethoxy; isopropyl; 1-hydroxy-2-methylpropan-2-yl; 2-hydroxy-2-propyl; hydroxymethyl; 1-hydroxyethyl; 2-hydroxyethyl; 1-hydroxy-2-propyl; 1-hydroxy-1-cyclopropyl; COCH₃; COPh; 2-methoxy-2-propyl; methoxymethyl; (dimethylamino)methyl; S(O₂)CH₃; and S(O₂)NR¹¹R¹².
62. The compound of any one of embodiments 1-59, wherein one or more R¹ when present is independently a C₁-C₆ alkyl substituted with one or more hydroxy.
63. The compound of embodiment 62, wherein one or more R¹ is independently selected from 1-hydroxy-2-methylpropan-2-yl; 2-hydroxy-2-propyl; hydroxymethyl; 1-hydroxyethyl; 2-hydroxyethyl; 1-hydroxy-2-propyl; 1,2-dihydroxy-2-propyl; and 1,2,3-trihydroxy-2-propyl
64. The compound of any one of embodiments 1-59, wherein one or more R¹ when present is independently a C₁-C₆ alkyl substituted with one or more hydroxy and further substituted with one or more (e.g., one) NR⁸R⁹ or R¹⁵.
65. The compound of embodiment 64, wherein one or more R¹ is independently selected from 1-amino-2-hydroxy-prop-2-yl; 1-acetamido-2-hydroxy-prop-2-yl; 1-(tert-butoxycarbonyl)amino-2-hydroxy-prop-2-yl; 1-(2-hydroxyethoxy)-2-hydroxy-2-propyl; 1-(2-benzyloxyethoxy)-2-hydroxy-2-propyl; and 1-(2-methoxyethoxy)-2-hydroxy-2-propyl.
66. The compound of any one of embodiments 1-59, wherein one or more R¹ when present is independently a C₁-C₆ alkyl optionally substituted with one or more halo.
67. The compound of embodiment 66, wherein one or more R¹ is independently selected from ethyl or difluoromethyl.
68. The compound of any one of embodiments 1-59, wherein one or more R¹ is independently C₁-C₆ alkyl substituted with one or more (e.g., one) NR⁸R⁹ and further optionally substituted with one or more halo.
69. The compound of embodiment 68, wherein one or more R¹ is independently selected from: (methylamino)methyl; (2,2-difluoroeth-1-yl)(methyl)aminomethyl; (2,2,2-trifluoroeth-1-yl)(methyl)aminomethyl; (dimethylamino)methyl; 1-(dimethylamino)ethyl; 2-((methyl)aminomethyl)-prop-2-yl; 2-((methyl)amino)-prop-2-yl; (methyl)(cyclopropylmethyl)aminomethyl; (methyl)(2-(dimethylamino)eth-1-yl)aminomethyl; (cyclobutyl)(methyl)aminomethyl; 1-(cyclobutyl)amino-eth-1-yl; isopropylaminomethyl; (cyclobutyl)aminomethyl; cycloheptylaminomethyl; tetrahydropyranylaminomethyl; sec-butylaminomethyl; ethylaminomethyl; allylaminomethyl; (2,2-difluoroeth-1-yl)aminomethyl; (2-methoxy-eth-1-yl)aminomethyl; (2-methoxy-eth-1-yl)(methyl)aminomethyl; 2-fluoro-1-dimethylamino-eth-1-yl; 1-dimethylamino-2,2-difluoroeth-1-yl; 1-dimethylamino-2,2,2-trifluoroeth-1-yl; 1-dimethylamino-2,2,2-trimethyleth-1-yl; and dimethylamino(cyclopropyl)methyl (e.g., one or more R¹ is dimethylaminomethyl or methylaminomethyl).
70. The compound of any one of embodiments 62-69, wherein one or more R² is independently selected from C₁-C₆ alkyl; C₁-C₆ alkyl optionally substituted with one or more hydroxyl; C₁-C₆ alkyl optionally substituted with one or more C₁-C₆ alkoxy; and halo.
71. The compound of any one of embodiments 1-19, 36-37, 40-41, 43-47, 50, 53, and 55-58, wherein one pair of R¹ and R² is on adjacent atoms, and taken together with the atoms connecting them, independently form one monocyclic or bicyclic C₄-C₁₂ carbocyclic ring (e.g., C₅ or C₆ carbocyclic ring) or one monocyclic or bicyclic 5- to-12-membered heterocyclic ring containing 1-3 (e.g., 1-2, e.g., 2) heteroatoms independently selected from O, N, and S (e.g., tetrahydropyridine, dihydrofuran, or dihydropyran), wherein the carbocyclic ring or heterocyclic ring is optionally independently substituted with one or more substituents each independently selected from hydroxy, halo, oxo, C₁-C₆ alkyl (e.g., methyl), C₂-C₆ alkenyl, C₂-C₆ alkynyl, C₁-C₆ alkoxy (e.g., methoxy, ethoxy, isopropoxyl), OC₃-C₁₀ cycloalkyl, NR⁸R⁹, ═NR¹⁰, CN, COOC₁-C₆ alkyl, S(O₂)C₆-C₁₀ aryl, C₆-C₁₀ aryl, 5- to 10-membered heteroaryl, C₃-C₁₀ cycloalkyl, 3- to 10-membered heterocycloalkyl (e.g., azetidinyl or oxetanyl), and CONR⁸R⁹, wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo (e.g., fluoro), C₁-C₆ alkyl, C₂-C₆ alkenyl, C₂-C₆ alkynyl, C₃-C₁₀ cycloalkyl, C₁-C₆ alkoxy, oxo, NR⁸R⁹ (e.g., amino, methylamino, or dimethylamino), ═NR¹⁰, COOC₁-C₆ alkyl, C₆-C₁₀ aryl, and CONR⁸R⁹.
72. The compound of any one of embodiments 1-19, 36-37, 40-41, 43-47, 50, 53, and 55-58 wherein one pair of R¹ and R² is on adjacent atoms, and taken together with the atoms connecting them, independently form one monocyclic or bicyclic C₅-C₆ carbocyclic ring,

wherein the carbocyclic is optionally independently substituted with one or more substituents each independently selected from hydroxy, halo, oxo, methyl, isopropoxyl, azetidinyl, oxetanyl, wherein the methyl, isopropoxyl, azetidinyl, and oxetanyl are optionally substituted with one or more substituents each independently selected from hydroxy, fluoro, amino, methylamino, and dimethylamino; or

one pair of R¹ and R² on adjacent atoms taken together forms a moiety selected from:

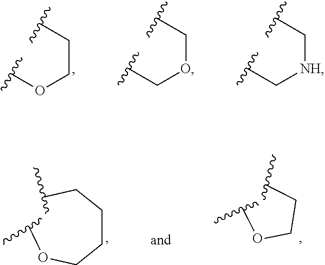 [SMILES: *CCO* ; *COC* ; *CNC* ; *CCCCO* ; *CCO*]

each of which is optionally independently substituted with one or more substituents each independently selected from hydroxy, halo, oxo, methyl, isopropoxyl, azetidinyl, oxetanyl, wherein the methyl, isopropoxyl, azetidinyl, and oxetanyl are optionally substituted with one or more substituents each independently selected from hydroxy, fluoro, amino, methylamino, and dimethylamino.
73. The compound of any one of embodiments 1-19, 36-37, 40-41, 43-47, 50, 53, and 55-58, wherein one pair of R¹ and R² is on adjacent atoms, and taken together with the atoms connecting them, independently form at least one bicyclic spirocyclic C₄-C₁₂ carbocyclic ring, wherein the carbocyclic ring is optionally substituted with one or more substituents each independently selected from hydroxy, halo, oxo, methyl, isopropoxyl, azetidinyl, oxetanyl, wherein the methyl, isopropoxyl, azetidinyl, and oxetanyl are optionally substituted with one or more substituents each independently selected from hydroxy, fluoro, amino, methylamino, and dimethylamino.
74. The compound of any one of embodiments 1-19, 36-37, 40-41, 43-47, 50, 53, and 55-58, wherein one pair of R¹ and R² is on adjacent atoms, and taken together with the atoms connecting them, independently form at least one bicyclic spirocyclic 5- to-12-membered heterocyclic ring containing 1-3 heteroatoms independently selected from O, N, and S, wherein the carbocyclic or heterocyclic ring is optionally substituted with one or more substituents each independently selected from hydroxy, halo, oxo, methyl, isopropoxyl, azetidinyl, oxetanyl, wherein the methyl, isopropoxyl, azetidinyl, and oxetanyl are optionally substituted with one or more substituents each independently selected from hydroxy, fluoro, amino, methylamino, and dimethylamino.
75. The compound of any one of embodiments 1-11, wherein one pair of R¹ and R² are on adjacent atoms, and taken together with the atoms connecting them, independently form one monocyclic or bicyclic C₄-C₁₂ carbocyclic ring or one monocyclic or bicyclic 5- to-12-membered heterocyclic ring containing 1-3 heteroatoms independently selected from O, N, and S, wherein the carbocyclic ring or heterocyclic ring is optionally independently substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR⁸R⁹, ═NR¹⁰, CN, COOC₁-C₆ alkyl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR⁸R⁹, wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR⁸R⁹, ═NR¹⁰, COOC₁-C₆ alkyl, C₆-C₁₀ aryl, and CONR⁸R⁹.
76. The compound of any one of embodiments 1-11, wherein the optionally substituted ring A is a pyrazolyl; m is 1; n is 1; R¹ and R² are on adjacent atoms, and taken together with the atoms connecting them, independently form one monocyclic or bicyclic C₄-C₁₂ carbocyclic ring or one monocyclic or bicyclic 5- to-12-membered heterocyclic ring containing 1-3 heteroatoms independently selected from O, N, and S, wherein the carbocyclic ring or heterocyclic ring is optionally independently substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR⁸R⁹, ═NR¹⁰, CN, COOC₁-C₆ alkyl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR⁸R⁹, wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR⁸R⁹, ═NR¹⁰, COOC₁-C₆ alkyl, C₆-C₁₀ aryl, and CONR⁸R⁹.
77. The compound of any one of embodiments 1-11, wherein the optionally substituted ring A is an imidazolyl; m is 1; n is 1; R¹ and R² are on adjacent atoms, and taken together with the atoms connecting them, independently form one monocyclic or bicyclic C₄-C₁₂ carbocyclic ring or one monocyclic or bicyclic 5- to-12-membered heterocyclic ring containing 1-3 heteroatoms independently selected from O, N, and S, wherein the carbocyclic ring or heterocyclic ring is optionally independently substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR⁸R⁹, ═NR¹⁰, CN, COOC₁-C₆ alkyl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR⁸R⁹, wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR⁸R⁹, ═NR¹⁰, COOC₁-C₆ alkyl, C₆-C₁₀ aryl, and CONR⁸R⁹.
78. The compound of any one of embodiments 1-11, wherein the optionally substituted ring A is a thiophenyl; m is 1; n is 1; R¹ and R² are on adjacent atoms, and taken together with the atoms connecting them, independently form one monocyclic or bicyclic C₄-C₁₂ carbocyclic ring or one monocyclic or bicyclic 5- to-12-membered heterocyclic ring containing 1-3 heteroatoms independently selected from O, N, and S, wherein the carbocyclic ring or heterocyclic ring is optionally independently substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR⁸R⁹, ═NR¹⁰, CN, COOC₁-C₆ alkyl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR⁸R⁹, wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR⁸R⁹, ═NR¹⁰, COOC₁-C₆ alkyl, C₆-C₁₀ aryl, and CONR⁸R⁹.
79. The compound of any one of embodiments 1-11, wherein the optionally substituted ring A is a thiazolyl; m is 1; n is 1; R¹ and R² are on adjacent atoms, and taken together with the atoms connecting them, independently form one monocyclic or bicyclic C₄-C₁₂ carbocyclic ring or one monocyclic or bicyclic 5- to-12-membered heterocyclic ring containing 1-3 heteroatoms independently selected from O, N, and S, wherein the carbocyclic ring or heterocyclic ring is optionally independently substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR⁸R⁹, ═NR¹⁰, CN, COOC₁-C₆ alkyl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR⁸R⁹, wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR⁸R⁹, ═NR¹⁰, COOC₁-C₆ alkyl, C₆-C₁₀ aryl, and CONR⁸R⁹.
80. The compound of any one of embodiments 1-11, wherein the optionally substituted ring A is

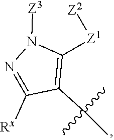

wherein R^{x} is selected from the group consisting of H and C₁-C₆ alkyl (e.g., methyl); Z¹ is selected from the group consisting of O, NH, and —CH₂— optionally substituted with 1-2 R²⁰; Z² is selected from the group consisting of NH and —CH₂— optionally substituted with 1-2 R²⁰; Z³ is selected from the group consisting of —CH₂— optionally substituted with 1-2 R²⁰, —CH₂CH₂— optionally substituted with 1-2 R²⁰, and —CH₂CH₂CH₂— optionally substituted with 1-2 R²⁰; R²⁰ is selected from the group consisting of hydroxy, halo (e.g., fluoro), oxo, C₁-C₆ alkyl (e.g., methyl or ethyl) optionally substituted with one R²¹, C₁-C₆ alkoxy (e.g., methoxy, ethoxy, or isopropoxy) optionally substituted with one R²¹, NR⁸R⁹, 3- to 10-membered heterocycloalkyl (e.g., azetidinyl or pyrrolidinyl) optionally substituted with one R²¹, or one pair of R²⁰ on the same atom, taken together with the atom connecting them, independently forms a monocyclic C₃-C₄ carbocyclic ring or a monocyclic 3- to 4-membered heterocyclic ring containing 1 O atom optionally substituted with OS(O)₂Ph; R²¹ is selected from the group consisting of halo (e.g., fluoro), NR⁸R⁹, C₂-C₆ alkynyl (e.g., ethynyl), and C₁-C₆ alkoxy (e.g., methoxy); R⁸ and R⁹ at each occurrence is independently selected from hydrogen, C₁-C₆ alkyl (e.g., methyl or ethyl), COR¹³, and CO₂R¹³; R¹³ is selected from the group consisting of: C₁-C₆ alkyl (e.g., methyl or t-butyl) and C₁-C₆ haloalkyl (e.g., trifluoromethyl).
81. The compound of any one of embodiments 1-11, wherein the optionally substituted ring A is

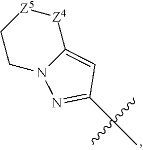

wherein Z⁴ is selected from the group consisting of —CH₂—, —C(O)—, and NH; Z⁵ is selected from the group consisting of O, NH, N—CH₃, and —CH₂—.
82. The compound of any of embodiments 1-2 and 4-81, wherein B is phenyl substituted with 1 or 2 R⁶ and optionally substituted with 1, 2, or 3 R⁷.
83. The compound of embodiment 82, wherein o=2 and p=0.
84. The compound of any one of embodiments 82-83, wherein B is

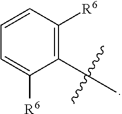

85. The compound of embodiment 84, wherein each R⁶ is independently selected from the group consisting of: C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO-C₁-C₆ alkyl; CONR⁸R⁹, and 4- to 6-membered heterocycloalkyl, wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl.
86. The compound of any one of embodiments 84 and 85, wherein each R⁶ is independently selected from the group consisting of: C₁-C₆ alkyl, C₃-C₇ cycloalkyl, C₁-C₆ haloalkyl, C₁-C₆ alkoxy, C₁-C₆ haloalkoxy, wherein the C₁-C₆ alkyl, C₁-C₆ haloalkyl, and C₃-C₇ cycloalkyl is optionally substituted with one or more substituents each independently selected from hydroxy, halo, CN, or oxo.
87. The compound of embodiment 82, wherein o=1 and p=1.
88. The compound of embodiment 82, wherein o=2 and p=1.
89. The compound of embodiment 88, wherein B is

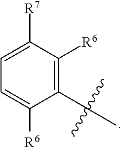

90. The compound of embodiment 88, wherein B is

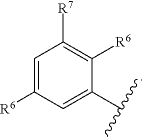

91. The compound of embodiment 88, wherein B is

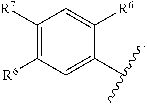

92. The compound of any one of embodiments 89-91, wherein each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO-C₁-C₆ alkyl; CONR⁸R⁹, and 4- to 6-membered heterocycloalkyl,

wherein the C₁-C₆ alkyl, C₁-C₆ haloalkyl, C₆-C₁₀ aryl, 5- to 10-membered heteroaryl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;

wherein R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy;

or R⁶ and R⁷, taken together with the atoms connecting them, independently form C₄-C₇ carbocyclic ring or at least one 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.
93. The compound of embodiment 88, wherein B is

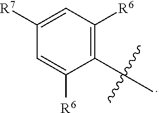

94. The compound of embodiment 93, wherein each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO-C₁-C₆ alkyl; CONR⁸R⁹, and 4- to 6-membered heterocycloalkyl,

wherein the C₁-C₆ alkyl, C₁-C₆ haloalkyl, C₆-C₁₀ aryl, 5- to 10-membered heteroaryl, C₃-C₇ cycloalkyl, and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;

wherein R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy.
95. The compound of embodiment 82, wherein o=2 and p=2.
96. The compound of embodiment 95, wherein B is

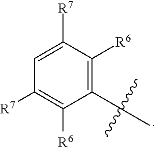

97. The compound of embodiment 95, wherein B is

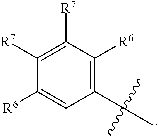

98. The compound of any one of embodiments 96-97, wherein each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO-C₁-C₆ alkyl; CONR⁸R⁹, and 4- to 6-membered heterocycloalkyl,

wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;

wherein each R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy;

or at least one pair of R⁶ and R⁷ on adjacent atoms, taken together with the atoms connecting them, independently form at least one C₄-C₇ carbocyclic ring or at least one 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.
99. The compound of any one of embodiments 95-98, wherein two pairs, each of one R⁶ and one R⁷, are on adjacent atoms, and each pair of one R⁶ and one R⁷ taken together with the atoms connecting them independently form a C₄-C₇ carbocyclic ring or a 5- to 7-membered heterocyclic ring containing 1 or 2 heteroatoms independently selected from O, N, and S, wherein each carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.
100. The compound of any one of embodiments 96-99, wherein one pair of R⁶ and R⁷ taken together with the atoms connecting them independently form a C5 carbocyclic ring, wherein the carbocyclic ring is optionally substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.
101. The compound of embodiment 100, wherein the other pair of R⁶ and R⁷ taken together with the atoms connecting them independently form a C₅ carbocyclic ring, wherein the carbocyclic ring is optionally substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.
102. The compound of any one of embodiments 96 and 99-101, wherein the substituted ring B is:

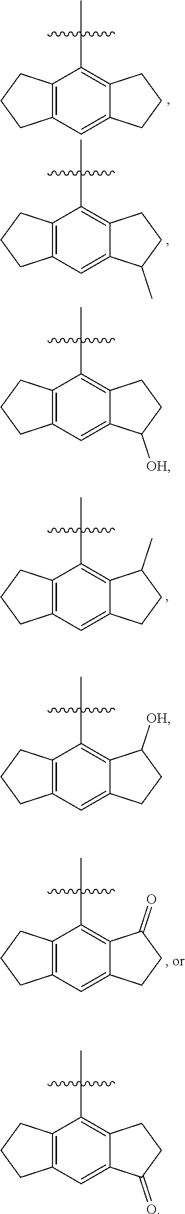

103. The compound of any one of embodiments 97 and 99-101, wherein the substituted ring B is:

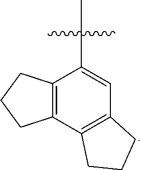 [SMILES: *c1cc2c(c3c1CCC3)CCC2]

104. The compound of embodiment 100, wherein the other pair of R⁶ and R⁷ taken together with the atoms connecting them independently form a C₄ or C₆₋₇ (e.g., C₄) carbocyclic ring, wherein the carbocyclic ring is optionally substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.
105. The compound of embodiment 104, wherein the substituted ring B is:

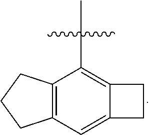 [SMILES: *c1c2c(cc3c1CC3)CCC2]

106. The compound of any one of embodiments 96-99, wherein one pair of R⁶ and R⁷ taken together with the atoms connecting them independently form a C₄ carbocyclic ring, wherein the carbocyclic ring is optionally substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.
107. The compound of embodiment 106, wherein the other pair of R⁶ and R⁷ taken together with the atoms connecting them independently form a C₄ carbocyclic ring, wherein the carbocyclic ring is optionally substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.
108. The compound of embodiment 107, wherein the substituted ring B is:

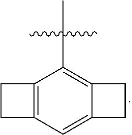 [SMILES: *c1c2c(cc3c1CC3)CC2]

109. The compound of any one embodiments 96 and 98, wherein one pair of R⁶ and R⁷ on adjacent atoms, taken together with the atoms connecting them, independently form one C₄-C₇ carbocyclic ring or one 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹; and each of the remaining R⁶ and R⁷ is independently selected from C₁-C₆ alkyl, C₁-C₆ haloalkyl, and C₃-C₇ cycloalkyl.
110. The compound of embodiment 109, wherein one pair of R⁶ and R⁷ on adjacent atoms, taken together with the atoms connecting them, independently form one C₄-C₇ (e.g., C₅) carbocyclic ring; and each of the remaining R⁶ and R⁷ is independently selected from C₁-C₆ alkyl, C₁-C₆ haloalkyl, and C₃-C₇ cycloalkyl.
111. The compound of embodiment 110, wherein the substituted ring B is

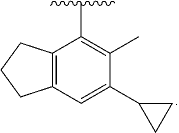 [SMILES: *c1c(C)c(C2CC2)cc2c1CCC2]

112. The compound of embodiment 95, wherein B is

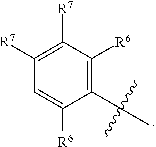

113. The compound of embodiment 112, wherein each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO-C₁-C₆ alkyl; CONR⁸R⁹, and 4- to 6-membered heterocycloalkyl,

wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;

wherein each R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy;

or R⁶ and R⁷, taken together with the atoms connecting them, independently form C₄-C₇ carbocyclic ring or at least one 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.
114. The compound of embodiment 82, wherein o=2 and p=3
115. The compound of embodiment 114, wherein B is

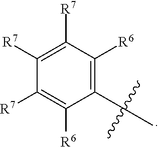

116. The compound of embodiment 115, wherein each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO-C₁-C₆ alkyl; CONR⁸R⁹, and 4- to 6-membered heterocycloalkyl,

wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;

wherein each R⁷ is independently selected from C₁-C₆ alkyl, C₂-C₆ alkenyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy;

or at least one pair of R⁶ and R⁷ on adjacent atoms, taken together with the atoms connecting them, independently form at least one C₄-C₇ carbocyclic ring or at least one 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.
117. The compound of any one of embodiments 114-116, wherein two pairs, each of one R⁶ and one R⁷, are on adjacent atoms, and each pair of one R⁶ and one R⁷ taken together with the atoms connecting them independently form a C₄-C₇ carbocyclic ring or a 5- to 7-membered heterocyclic ring containing 1 or 2 heteroatoms independently selected from O, N, and S, wherein each carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.
118. The compound of any one of embodiments 114-117, wherein one pair of R⁶ and R⁷ taken together with the atoms connecting them independently form a C₅ carbocyclic ring, wherein the carbocyclic ring is optionally substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.
119. The compound of embodiment 118, wherein the other pair of R⁶ and R⁷ taken together with the atoms connecting them independently form a C₅ carbocyclic ring, wherein the carbocyclic ring is optionally substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.
120. The compound of embodiment 119, wherein the substituted ring B is:

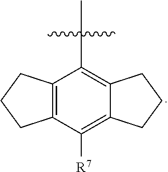

121. The compound of embodiment 120, wherein R⁷ is selected from each R⁷ is independently selected from C₁-C₆ alkyl, C₂-C₆ alkenyl, C₁-C₆ haloalkyl, C₁-C₆ alkoxy, C₁-C₆ haloalkoxy, halo, and CN.
122. The compound of 3, wherein the

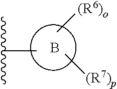

moiety is as defined for (BB-2).
123. The compound of embodiment 122, wherein o=2 and p=1.
124. The compound of embodiment 123, wherein B is

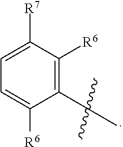

125. The compound of embodiment 123, wherein B is

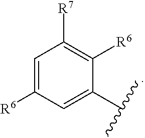

126. The compound of embodiment 123, wherein B is

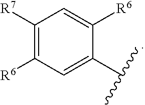

127. The compound of any one of embodiments 124-126, wherein each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO-C₁-C₆ alkyl; CONR⁸R⁹, and 4- to 6-membered heterocycloalkyl,

wherein the C₁-C₆ alkyl, C₁-C₆ haloalkyl, C₆-C₁₀ aryl, 5- to 10-membered heteroaryl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;

wherein R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy;

or R⁶ and R⁷, taken together with the atoms connecting them, independently form C₄-C₇ carbocyclic ring or at least one 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.
128. The compound of embodiment 123, wherein B is

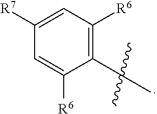

129. The compound of embodiment 128, wherein each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO-C₁-C₆ alkyl; CONR⁸R⁹, and 4- to 6-membered heterocycloalkyl,

wherein the C₁-C₆ alkyl, C₁-C₆ haloalkyl, C₆-C₁₀ aryl, 5- to 10-membered heteroaryl, C₃-C₇ cycloalkyl, and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;

wherein R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy.
130. The compound of embodiment 122, wherein o=2 and p=2.
131. The compound of embodiment 130, wherein B is

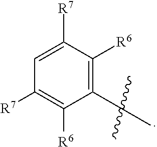

132. The compound of embodiment 130, wherein B is

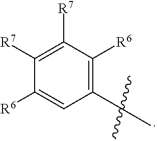

133. The compound of any one of embodiments 131-132, wherein each R⁶ is independently selected from C1-C6alkyl, C₃-C₇cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO-C₁-C₆ alkyl; CONR⁸R⁹, and 4- to 6-membered heterocycloalkyl,

wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;

wherein each R⁷ is independently selected from C₁-C₆ alkyl, C₁-C₆ haloalkyl, C₁-C₆ alkoxy, C haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy;

or at least one pair of R⁶ and R⁷ on adjacent atoms, taken together with the atoms connecting them, independently form at least one C₄-C₇ carbocyclic ring or at least one 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹; provided that:

when two pairs of R⁶ and R⁷ are on adjacent atoms; and each pair taken together with the atoms connecting them, independently forms a ring selected from a C₄-C₈ carbocyclic ring and a 5- to 8-membered heterocyclic ring containing 1 or 2 heteroatoms independently selected from O, N, and S,

then at least one ring is selected from:

(a) a C₄ carbocyclic ring, a C₆-C₈ carbocyclic ring, or a 5- to 8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹; and

(b) a C₅ carbocyclic ring substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹;
134. The compound of any one of embodiments 131-133, wherein two pairs, each of one R⁶ and one R⁷, are on adjacent atoms, and each pair of one R⁶ and one R⁷ taken together with the atoms connecting them independently form a C₄-C₇ carbocyclic ring or a 5- to 7-membered heterocyclic ring containing 1 or 2 heteroatoms independently selected from O, N, and S, wherein each carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.
135. The compound of any one of embodiments 131-134, wherein one pair of R⁶ and R⁷ taken together with the atoms connecting them independently form a C₅ carbocyclic ring, wherein the carbocyclic ring is optionally substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.
136. The compound of embodiment 135, wherein the other pair of R⁶ and R⁷ taken together with the atoms connecting them independently form a C₅ carbocyclic ring, wherein the carbocyclic ring is substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.
137. The compound of any one of embodiments 131 and 135-136, wherein the substituted ring B is:

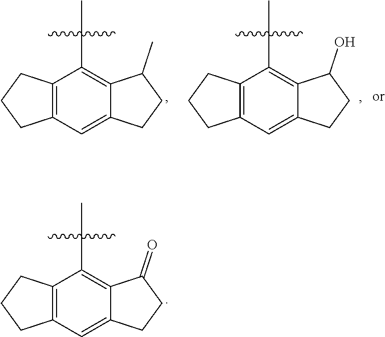

138. The compound of any one of embodiments 131 and 135-136, wherein the substituted ring B is:

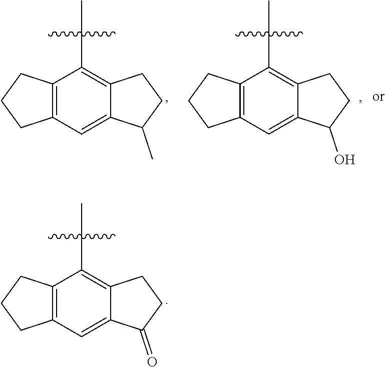

139. The compound of embodiment 135, wherein the other pair of R⁶ and R⁷ taken together with the atoms connecting them independently form a C₄ or C₆₋₇ (e.g., C4) carbocyclic ring, wherein the carbocyclic ring is optionally substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.
140. The compound of embodiment 139, wherein the substituted ring B is:

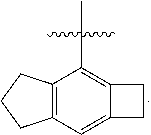 [SMILES: *c1c2c(cc3c1CC3)CCC2]

141. The compound of any one of embodiments 131-134, wherein one pair of R⁶ and R⁷ taken together with the atoms connecting them independently form a C₄ carbocyclic ring, wherein the carbocyclic ring is optionally substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.
142. The compound of embodiment 141, wherein the other pair of R⁶ and R⁷ taken together with the atoms connecting them independently form a C₄ carbocyclic ring, wherein the carbocyclic ring is optionally substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.
143. The compound of embodiment 142, wherein the substituted ring B is:

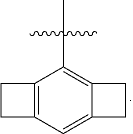 [SMILES: *c1c2c(cc3c1CC3)CC2]

144. The compound of any one embodiments 131-133, wherein one pair of R⁶ and R⁷ on adjacent atoms, taken together with the atoms connecting them, independently form one C₄-C₇ carbocyclic ring or one 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹; and each of the remaining R⁶ and R⁷ is independently selected from C₁-C₆ alkyl, C₁-C₆ haloalkyl, and C₃-C₇ cycloalkyl.
145. The compound of embodiment 144, wherein one pair of R⁶ and R⁷ on adjacent atoms, taken together with the atoms connecting them, independently form one C₄-C₇ (e.g., C₅) carbocyclic ring; and each of the remaining R⁶ and R⁷ is independently selected from C₁-C₆ alkyl, C₁-C₆ haloalkyl, and C₃-C₇ cycloalkyl.
146. The compound of embodiment 145, wherein the substituted ring B is

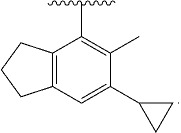 [SMILES: *c1c(C)c(C2CC2)cc2c1CCC2]

147. The compound of embodiment 130, wherein B is

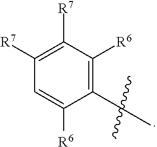

148. The compound of embodiment 147, wherein each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO-C₁-C₆ alkyl; CONR⁸R⁹, and 4- to 6-membered heterocycloalkyl,

wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;

wherein each R⁷ is independently selected from C₁-C₆ alkyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy;

or R⁶ and R⁷, taken together with the atoms connecting them, independently form C₄-C₇ carbocyclic ring or at least one 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.
149. The compound of embodiment 122, wherein o=2 and p=3
150. The compound of embodiment 149, wherein B is

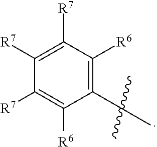

151. The compound of embodiment 150, wherein each R⁶ is independently selected from C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO-C₁-C₆ alkyl; CONR⁸R⁹, and 4- to 6-membered heterocycloalkyl,

wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl;

wherein each R⁷ is independently selected from C₁-C₆ alkyl, C₂-C₆ alkenyl, C₁-C₆ haloalkyl, C₁-C₆ alkoxy, C₁-C₆ haloalkoxy, halo, CN, COC₁-C₆ alkyl, CO₂C₁-C₆ alkyl, CO₂C₃-C₆ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, CONR⁸R⁹, SF₅, S(O₂)C₁-C₆ alkyl, C₃-C₇ cycloalkyl and 4- to 6-membered heterocycloalkyl, wherein the C₁-C₆ alkyl is optionally substituted with one to two C₁-C₆ alkoxy;

or at least one pair of R⁶ and R⁷ on adjacent atoms, taken together with the atoms connecting them, independently form at least one C₄-C₇ carbocyclic ring or at least one 5-to-7-membered heterocyclic ring containing 1 or 2 heteroatoms independently selected from O, N, and S, wherein the carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.
152. The compound of any one of embodiments 149-151, wherein two pairs, each of one R⁶ and one R⁷, are on adjacent atoms, and each pair of one R⁶ and one R⁷ taken together with the atoms connecting them independently form a C₄-C₇ carbocyclic ring or a 5- to 7-membered heterocyclic ring containing 1 or 2 heteroatoms independently selected from O, N, and S, wherein each carbocyclic ring or heterocyclic ring is optionally independently substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.
153. The compound of any one of embodiments 149-152, wherein one pair of R⁶ and R⁷ taken together with the atoms connecting them independently form a C₅ carbocyclic ring, wherein the carbocyclic ring is optionally substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.
154. The compound of embodiment 153, wherein the other pair of R⁶ and R⁷ taken together with the atoms connecting them independently form a C₅ carbocyclic ring, wherein the carbocyclic ring is optionally substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.
155. The compound of embodiment 154, wherein the substituted ring B is:

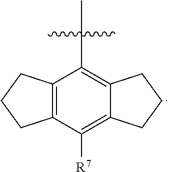

156. The compound of embodiment 155, wherein R⁷ is selected from each R⁷ is independently selected from C₁-C₆ alkyl, C₂-C₆ alkenyl, C₁-C₆ haloalkyl, C₁-C₆ alkoxy, C₁-C₆ haloalkoxy, halo, and CN.
157. The compound of any one of embodiments 1-82, wherein B is pyridyl, or an N-oxide thereof.
158. The compound of any one of embodiments 1-82 and 157, wherein the B is 3-pyridyl.
159. The compound of any one of embodiments 1-82 and 157-158, wherein o=2 and p=1.
160. The compound of any one of embodiments 159, wherein the substituted ring B is

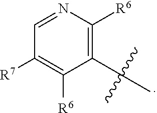

161. The compound of embodiment 160, wherein each R⁶ is independently selected from the group consisting of: C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO-C₁-C₆ alkyl; CONR⁸R⁹, and 4- to 6-membered heterocycloalkyl, wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl.
162. The compound of embodiment 161, wherein each R⁶ is independently selected from the group consisting of: C₁-C₆ alkyl, halo, C₃-C₇ cycloalkyl, and C₆-C₁₀ aryl.
163. The compound of embodiment 162, wherein each R⁶ is independently selected from the group consisting of: methyl, isopropyl, cyclopropyl, fluoro, and phenyl.
164. The compound of any one of embodiments 161-163, wherein each R⁷ is independently selected from the group consisting of: C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO-C₁-C₆ alkyl; CONR⁸R⁹, and 4- to 6-membered heterocycloalkyl, wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl.
165. The compound of any one of embodiments 161-164, wherein each R⁷ is independently selected from the group consisting of: C₁-C₆ alkyl, C₃-C₇ cycloalkyl, halo, and C₆-C₁₀ aryl.
166. The compound of embodiment 165, wherein each R⁷ is independently selected from the group consisting of: methyl, isopropyl, cyclopropyl, fluoro, and phenyl.
167. The compound of any one of embodiments 1-82 and 157, wherein the substituted ring B is 4-pyridyl.
168. The compound of any one of embodiments 1-82 and 167, wherein o=2 and p=0.
169. The compound of any one of embodiments 1-82 and 167-168, wherein the substituted ring B is

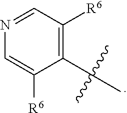

170. The compound of any one of embodiments 1-82 and 167-169, wherein each R⁶ is independently selected from the group consisting of: C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO-C₁-C₆ alkyl; CONR⁸R⁹, and 4- to 6-membered heterocycloalkyl, wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl.
171. The compound of any one of embodiments 1-82 and 167-170, wherein each R⁶ is independently selected from the group consisting of: C₁-C₆ alkyl and C₃-C₇ cycloalkyl.
172. The compound of embodiment 171, wherein each R⁶ is isopropyl.
173. The compound of any one of embodiments 1-82 and 167, wherein o=2 and p=2.
174. The compound of any one of embodiments 1-82, 167, and 173, wherein the substituted ring B is

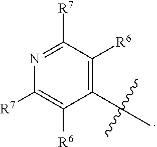

175. The compound of embodiment 174, wherein each R⁶ is independently selected from the group consisting of: C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO-C₁-C₆ alkyl; CONR⁸R⁹, and 4- to 6-membered heterocycloalkyl, wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl.
176. The compound of any one of embodiments 1-82, 167, and 174-175, wherein each R⁶ is independently selected from the group consisting of: C₁-C₆ alkyl, C₃-C₇ cycloalkyl, halo, and C₆-C₁₀ aryl.
177. The compound of embodiment 176, wherein each R⁶ is independently selected from the group consisting of: methyl, isopropyl, cyclopropyl, fluoro, and phenyl.
178. The compound of any one of embodiments 175-177, wherein each R⁷ is independently selected from the group consisting of: C₁-C₆ alkyl, C₃-C₇ cycloalkyl, C₁-C₆ haloalkyl, C₁-C₆ alkoxy, C₁-C₆ haloalkoxy, halo, CN, C₆-C₁₀ aryl, 5- to 10-membered heteroaryl, CO-C₁-C₆ alkyl; CONR⁸R⁹, and 4- to 6-membered heterocycloalkyl, wherein the C₁-C₆ alkyl, C₁-C₆ haloalkyl, C₃-C₇ cycloalkyl and 4- to 6-membered heterocycloalkyl is optionally substituted with one or more substituents each independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 4- to 6-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(4- to 6-membered heterocycloalkyl), NHCOC₁-C₆ alkyl, NHCOC₆-C₁₀ aryl, NHCO(5- to 10-membered heteroaryl), NHCO(4- to 6-membered heterocycloalkyl), and NHCOC₂-C₆ alkynyl.
179. The compound of embodiment 178, wherein each R⁷ is independently selected from the group consisting of: C₁-C₆ alkyl, C₃-C₇ cycloalkyl, halo, and C₆-C₁₀ aryl.
180. The compound of embodiment 179, wherein each R⁷ is independently selected from the group consisting of: methyl, isopropyl, cyclopropyl, fluoro, and phenyl.
181. The compound of any one of embodiments 1-82, 167, and 174-180, wherein one pair of R⁶ and R⁷ on adjacent atoms, taken together with the atoms connecting them, independently form at least one C₄-C₈ carbocyclic ring or at least one 5- to 8-membered heterocyclic ring containing 1 or 2 heteroatoms and/or heteroatomic groups independently selected from O, NH, NR¹³, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹.
182. The compound of any one of embodiments 1-82, 167, and 174-180, wherein one pair of R⁶ and R⁷ on adjacent atoms, taken together with the atoms connecting them, independently form at least one C₄-C₈ carbocyclic ring, wherein the carbocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹.
183. The compound of any one of embodiments 181-182, wherein the C₄-C₈ carbocyclic ring is a C₅ carbocyclic ring optionally substituted with one or more oxo, CH₃, or hydroxy.
184. The compound of embodiment 183, wherein the C₅ carbocyclic ring is substituted with one CH₃.
185. The compound of embodiment 183, wherein the C₅ carbocyclic ring is geminally substituted with two CH₃.
186. The compound of any one of embodiments 181-182, wherein the C₄-C₈ carbocyclic ring is a C₇ carbocyclic ring, wherein the C₇ carbocyclic ring is a bicyclic spirocycle, wherein the bicyclic spirocycle comprises a 5-membered ring and a 3-membered ring.
187. The compound of any one of embodiments 1-82 and 174, wherein the substituted ring B is

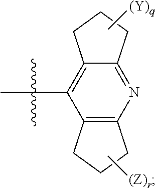

q is 0, 1, or 2; r is 0, 1, or 2; wherein each of Y and Z is independently selected from C₁-C₆ alkyl (e.g., methyl) and hydroxy; or wherein when two Y are attached to the same carbon, the two Y are taken together with the carbon they are attached to to form a cyclopropyl ring; or wherein when two Z are attached to the same carbon, the two Z are taken together with the carbon they are attached to form a cyclopropyl ring.
188. The compound of any one of embodiments 1-82 and 167, wherein the substituted ring B is

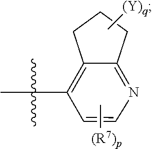

R⁷ is selected from C₁-C₆ alkyl (e.g., methyl, ethyl, or isopropyl), C₆-C₁₀ aryl (e.g., phenyl), and C₃-C₁₀ cycloalkyl (e.g., cyclopropyl); p is 0, 1, or 2; q is 0, 1, or 2; wherein each Y is independently selected from C₁-C₆ alkyl (e.g., methyl) and hydroxy; or when two Y are attached to the same carbon, the two Y are taken together with the carbon they are attached to to form a cyclopropyl ring.
189. The compound of any one of embodiments 1-82 and 167, wherein the substituted ring B is

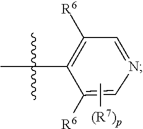

each R⁶ is independently selected from C₁-C₆ alkyl (e.g., isopropyl); each R⁷ is independently selected from halo (e.g., fluoro); p is 0 or 1.
190. The compound of any one of embodiments 1-82 and 158, wherein the substituted ring B is

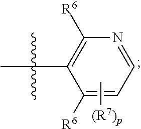

each R⁶ is independently selected from C₁-C₆ alkyl (e.g., isopropyl); each R⁷ is independently selected from halo (e.g., fluoro); p is 0 or 1.
191. The compound of any one of embodiments 1-82 and 167, wherein the substituted ring B is

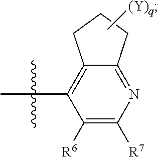

R⁶ is selected from C₁-C₆ alkyl (e.g., methyl, ethyl, or isopropyl) and C₃-C₁₀ cycloalkyl (e.g., cyclopropyl); R⁷ is selected from C₁-C₆ alkyl (e.g., methyl, ethyl, or isopropyl), C₁-C₆ haloalkyl (e.g., trifluoromethyl) and C₃-C₁₀ cycloalkyl (e.g., cyclopropyl or cyclobutyl); or R⁶ and R⁷, taken together with the atoms connecting them, independently form a C₅ carbocyclic ring optionally substituted with one or more C₁-C₆ alkyl (e.g., methyl); q is 0, 1, or 2; each Y is independently selected from C₁-C₆ alkyl (e.g., methyl); or when two Y are attached to the same carbon, the two Y are taken together with the carbon they are attached to to form a cyclopropyl ring.
192. The compound of any one of the preceding embodiments, wherein L^{A} is a bond.
193. The compound of any one of embodiments 1-191, wherein L^{A} is CH₂.
194. The compound of any one of the preceding embodiments, wherein the sulfur in the moiety S(═O)(NHR³)═N— has (S) stereochemistry.
195. The compound of any one of embodiments 1 to 194, wherein the sulfur in the moiety S(═O)(NHR³)═N— has (R) stereochemistry.
196. A compound selected from any examples defined herein, and a pharmaceutically acceptable salt thereof.
197. A pharmaceutical composition comprising a compound or salt as embodimented in any one of embodiments 1-196 and one or more pharmaceutically acceptable excipients.
198. A method for modulating NLRP3 activity, the method comprising contacting NLRP3 with an effective amount of a compound as embodimented in any one of embodiments 1-196 or a pharmaceutical composition as embodimented in embodiment 197.
199. The method of embodiment 198, wherein the modulating comprises antagonizing NLRP3.
200. The method of any one of embodiments 198 or 199, which is carried out in vitro.
201. The method of embodiment 198 to 200, wherein the method comprises contacting a sample comprising one or more cells comprising NLRP3 with the compound.
202. The method of any one of embodiments 198, 199 or 201, which is carried out in vivo.
203. The method of embodiment 202, wherein the method comprises administering the compound to a subject having a disease in which NLRP3 signaling contributes to the pathology and/or symptoms and/or progression of the disease.
204. The method of embodiment 203, wherein the subject is a human.
205. A method of treating a disease, disorder or condition that is a metabolic disorder, comprising administering to a subject in need of such treatment an effective amount of a compound as embodimented in any one of embodiments 1-196 or a pharmaceutical composition as embodimented in embodiment 197.
206. The method of embodiment 205, wherein the metabolic disorder is Type 2 diabetes, atherosclerosis, obesity or gout.
207. A method of treating a disease, disorder or condition that is a disease of the central nervous system, comprising administering to a subject in need of such treatment an effective amount of a compound as embodimented in any one of embodiments 1-196 or a pharmaceutical composition as embodimented in embodiment 197.
208. The method of embodiment 207, wherein the disease of the central nervous system is Alzheimer's disease, multiple sclerosis, Amyotrophic Lateral Sclerosis or Parkinson's disease.
209. A method of treating a disease, disorder or condition that is lung disease, comprising administering to a subject in need of such treatment an effective amount of a compound as embodimented in any one of embodiments 1-196 or a pharmaceutical composition as embodimented in embodiment 197.
210. The method of embodiment 209, wherein the lung disease is asthma, COPD or pulmonary idiopathic fibrosis.
211. A method of treating a disease, disorder or condition that is liver disease, comprising administering to a subject in need of such treatment an effective amount of a compound as embodimented in any one of embodiments 1-196 or a pharmaceutical composition as embodimented in embodiment 197.
212. The method of embodiment 211, wherein the liver disease is NASH syndrome, viral hepatitis or cirrhosis.
213. A method of treating a disease, disorder or condition that is pancreatic disease, comprising administering to a subject in need of such treatment an effective amount of a compound as embodimented in any one of embodiments 1-196 or a pharmaceutical composition as embodimented in embodiment 197.
214. The method of embodiment 213, wherein the pancreatic disease is acute pancreatitis or chronic pancreatitis.
215. A method of treating a disease, disorder or condition that is kidney disease, comprising administering to a subject in need of such treatment an effective amount of a compound as embodimented in any one of embodiments 1-196 or a pharmaceutical composition as embodimented in embodiment 197.
216. The method of embodiment 215, wherein the kidney disease is acute kidney injury or chronic kidney injury.
217. A method of treating a disease, disorder or condition that is intestinal disease, comprising administering to a subject in need of such treatment an effective amount of a compound as embodimented in any one of embodiments 1-196 or a pharmaceutical composition as embodimented in embodiment 197.
218. The method of embodiment 217, wherein the intestinal disease is Crohn's disease or Ulcerative Colitis.
219. A method of treating a disease, disorder or condition that is skin disease, comprising administering to a subject in need of such treatment an effective amount of a compound as embodimented in any one of embodiments 1-196 or a pharmaceutical composition as embodimented in embodiment 197.
220. The method of embodiment 219, wherein the skin disease is psoriasis.
221. A method of treating a disease, disorder or condition that is musculoskeletal disease, comprising administering to a subject in need of such treatment an effective amount of a compound as embodimented in any one of embodiments 1-196 or a pharmaceutical composition as embodimented in embodiment 197.
222. The method of embodiment 221, wherein the musculoskeletal disease is scleroderma.
223. A method of treating a disease, disorder or condition that is a vessel disorder, comprising administering to a subject in need of such treatment an effective amount of a compound as embodimented in any one of embodiments 1-196 or a pharmaceutical composition as embodimented in embodiment 197.
224. The method of embodiment 223, wherein the vessel disorder is giant cell arteritis.
225. A method of treating a disease, disorder or condition that is a disorder of the bones, comprising administering to a subject in need of such treatment an effective amount of a compound as embodimented in any one of embodiments 1-196 or a pharmaceutical composition as embodimented in embodiment 197.
226. The method of embodiment 225, wherein the disorder of the bones is osteoarthritis, osteoporosis or osteopetrosis disorders.
227. A method of treating a disease, disorder or condition that is eye disease, comprising administering to a subject in need of such treatment an effective amount of a compound as embodimented in any one of embodiments 1-196 or a pharmaceutical composition as embodimented in embodiment 197.
228. The method of embodiment 227, wherein the eye disease is glaucoma or macular degeneration.
229. A method of treating a disease, disorder or condition that is a disease caused by viral infection, comprising administering to a subject in need of such treatment an effective amount of a compound as embodimented in any one of embodiments 1-196 or a pharmaceutical composition as embodimented in embodiment 197.
230. The method of embodiment 229, wherein the diseases caused by viral infection is HIV or AIDS.
231. A method of treating a disease, disorder or condition that is an autoimmune disease, comprising administering to a subject in need of such treatment an effective amount of a compound as embodimented in any one of embodiments 1-196 or a pharmaceutical composition as embodimented in embodiment 197.
232. The method of embodiment 231, wherein the autoimmune disease is Rheumatoid Arthritis, Systemic Lupus Erythematosus, Autoimmune Thyroiditis.
233. A method of treating a disease, disorder or condition that is cancer or aging, comprising administering to a subject in need of such treatment an effective amount of a compound as embodimented in any one of embodiments 1-196 or a pharmaceutical composition as embodimented in embodiment 197.
234. A method of treating a disease, disorder or condition that is a cancer selected from: myelodysplastic syndromes (MDS); non-small cell lung cancer, such as non-small cell lung cancer in patients carrying mutation or overexpression of NLRP3; acute lymphoblastic leukemia (ALL), such as ALL in patients resistant to glucocorticoids treatment; Langerhan's cell histiocytosis (LCH); multiple myeloma; promyelocytic leukemia; acute myeloid leukemia (AML); chronic myeloid leukemia (CML); gastric cancer; and lung cancer metastasis, comprising administering to a subject in need of such treatment an effective amount of a compound as embodimented in any one of embodiments 1-196 or a pharmaceutical composition as embodimented in embodiment 197.
235. The method of embodiment 234, wherein the cancer is MDS.
236. The method of embodiment 234, wherein the cancer is non-small lung cancer.
237. The method of embodiment 234, wherein the cancer is acute lymphoblastic leukemia.
238. The method of embodiment 234, wherein the cancer is LCH.
239. The method of embodiment 234, wherein the cancer is multiple myeloma.
240. The method of embodiment 234, wherein the cancer is promyelocytic leukemia.
241. The method of embodiment 234, wherein the cancer is acute myeloid leukemia (AML).
242. The method of embodiment 234, wherein the cancer is chronic myeloid leukemia (CIVIL).
243. The method of embodiment 234, wherein the cancer is gastric cancer.
244. The method of embodiment 234, wherein the cancer is lung cancer metastasis.
245. The method of any one of embodiments 203-244, further comprising administering a therapeutically effective amount of an anti-TNFα agent to the subject.
246. The method of embodiment 245, wherein the NLRP3 antagonist is administered to the subject prior to administration of the anti-TNFα agent to the subject.
247. The method of embodiment 245, wherein the anti-TNFα agent is administered to the subject prior to the administration of the NLRP3 antagonist to the subject.
248. The method of embodiment 245, wherein the NLRP3 antagonist and the anti-TNFα agent are administered to the subject at substantially the same time.
249. The method of embodiment 245, wherein the NLRP3 antagonist and the anti-TNFα agent are formulated together in a single dosage form.

Where any discrepancies exist between the stereocenter descriptor used (i.e. R or S) and that depicted, the absolute stereochemistry depicted is relied upon as inaccuracies in the stereocenter descriptor may be generated due to inaccuracies in automatic nomenclature.

A number of embodiments of the invention have been described. Nevertheless, it will be understood that various modifications may be made without departing from the spirit and scope of the invention. Accordingly, other embodiments are within the scope of the following claims.

1. A compound of Formula AA 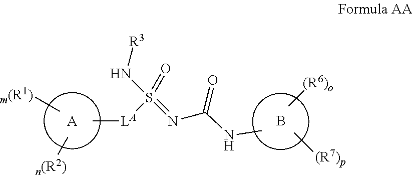
wherein
L^{A} is a bond or a C₁-C₈ alkylene optionally substituted with halo;
m=0, 1, or 2;
n=0, 1, or 2;
o=1 or 2;
p=0, 1, 2, or 3,
wherein
A is a 5- to 10-membered monocyclic or bicyclic heteroaryl or a C₆-C₁₀ monocyclic or bicyclic aryl;
B is a 5-10 membered monocyclic or bicyclic heteroaryl, or a C₆-C₁₀ monocyclic or bicyclic aryl; wherein at least one R⁶ is ortho to the bond connecting the B ring to the NH(CO) group of Formula AA;
R¹ and R² are each independently selected from C₁-C₆ alkyl, C₁-C₆ haloalkyl, C₁-C₆ alkoxy, C₁-C₆ haloalkoxy, halo, CN, NO₂, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NR⁸R⁹, C(O)R¹³, CONR⁸R⁹, SF₅, SC₁-C₆ alkyl, S(O₂)C₁-C₆ alkyl, S(O)C₁-C₆ alkyl, S(O₂)NR¹¹R¹², C₃-C₇ cycloalkyl, and 3- to 7-membered heterocycloalkyl,
wherein the C₁-C₆ alkyl, C₁-C₆ alkoxy, C₁-C₆ haloalkyl, C₆-C₁₀ aryl, 5- to 10-membered heteroaryl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, 10⁵, C₁-C₆ haloalkoxy, NR⁸R⁹, ═NR¹⁰, COOC₁-C₆ alkyl, CONR⁸R⁹, 3- to 7-membered heterocycloalkyl, C₃-C₇ cycloalkyl, C₆-C₁₀ aryl, 5- to 10-membered heteroaryl, OCOC₁-C₆ alkyl, OCOC₆-C₁₀ aryl, OCO(5- to 10-membered heteroaryl), and OCO(3- to 7-membered heterocycloalkyl);
wherein each C₁-C₆ alkyl substituent and each C₁-C₆ alkoxy substituent of the R¹ or R² C₃-C₇ cycloalkyl or of the R¹ or R² 3- to 7-membered heterocycloalkyl is further optionally independently substituted with one to three hydroxy, —O(C₀-C₃ alkylene)C₆-C₁₀ aryl, halo, NR⁸R⁹, or oxo;
wherein the 3- to 7-membered heterocycloalkyl, C₆-C₁₀ aryl, and 5- to 10-membered heteroaryl are optionally substituted with one or more substituents independently selected from halo, C₁-C₆ alkyl, and OC₁-C₆ alkyl;
or one pair of R¹ and R² on adjacent atoms, taken together with the atoms connecting them, independently form one monocyclic or bicyclic C₄-C₁₂ carbocyclic ring or one monocyclic or bicyclic 5- to-12-membered heterocyclic ring containing 1-3 heteroatoms independently selected from O, N, and S, wherein the carbocyclic ring or heterocyclic ring is optionally independently substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR⁸R⁹, ═NR¹⁰, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR⁸R⁹,
wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR⁸R⁹, ═NR¹⁰, COOC₁-C₆ alkyl, C₆-C₁₀ aryl, and CONR⁸R⁹;
R⁶ and R⁷ are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, C₃-C₁₀ cycloalkyl, 3- to 10-membered heterocycloalkyl, and C₂-C₆ alkenyl,
wherein R⁶ and R⁷ are each optionally substituted with one or more substituents independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), C₆-C₁₀ aryloxy, and S(O₂)C₁-C₆ alkyl; and wherein the C₁-C₆ alkyl or C₁-C₆ alkoxy that R⁶ or R⁷ is substituted with is optionally substituted with one or more hydroxyl, C₆-C₁₀ aryl or NR⁸R⁹, or wherein R⁶ or R⁷ is optionally fused to a five- to -seven-membered carbocyclic ring or heterocyclic ring containing one or two heteroatoms independently selected from oxygen, sulfur and nitrogen;
wherein the 3- to 7-membered heterocycloalkyl, C₆-C₁₀ aryl, 5- to 10-membered heteroaryl are optionally substituted with one or more substituents independently selected from halo, C₁-C₆ alkyl, and OC₁-C₆ alkyl;
or at least one pair of R⁶ and R⁷ on adjacent atoms, taken together with the atoms connecting them, independently form at least one C₄-C₈ carbocyclic ring or at least one 5- to 8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹;
R¹⁰ is C₁-C₆ alkyl;
each of R⁸ and R⁹ at each occurrence is independently selected from hydrogen, C₁-C₆ alkyl, C₂-C₆ alkenyl, C₃-C₇ cycloalkyl, (C═NR¹³)NR¹¹R¹², S(O₂)C₁-C₆ alkyl, S(O₂)NR¹¹R¹², COR¹³, CO₂R¹³ and CONR¹¹R¹²; wherein the C₁-C₆ alkyl is optionally substituted with one or more hydroxy, halo, C₁-C₆ alkoxy, C₆-C₁₀ aryl, 5- to 10-membered heteroaryl, C₃-C₇ cycloalkyl, 3- to 7-membered heterocycloalkyl, or NR¹¹R¹²;
or R⁸ and R⁹ taken together with the nitrogen they are attached to form a 3- to 10-membered monocyclic or bicyclic ring optionally containing one or more heteroatoms in addition to the nitrogen they are attached to, wherein the ring is optionally substituted with one or more substituents independently selected from halo, C₁-C₆ alkyl, C₁-C₆ haloalkyl, C₁-C₆ alkoxy, oxo, N(C₁-C₆ alkyl)₂, NH₂, NH(C₁-C₆ alkyl), and hydroxy;
R¹³ is C₁-C₆ alkyl, C₁-C₆ haloalkyl, or —(Z¹-Z²)ₐ₁-Z³;
each of R¹¹ and R¹² at each occurrence is independently selected from hydrogen, C₁-C₆ alkyl, and —(Z¹-Z²)ₐ₁-Z³;
a1 is 0-10 (e.g., 0-4);
each Z¹ is independently C₁-C₆ alkylene optionally substituted with one or more substituents independently selected from oxo, halo, and hydroxy;
each Z² is independently a bond, NH, N(C₁-C₆ alkyl), —O—, —S—, or 5-10 membered heteroarylene;
Z³ is independently C₆-C₁₀ aryl, C₂-C₆ alkenyl, C₂-C₆ alkynyl, C₃-C₁₀ cycloalkyl, 5- to 10-membered heteroaryl, or 3- to 10-membered heterocycloalkyl, each of which is optionally substituted with one or more substituents independently selected from halo, C₁-C₆ alkyl, C₁₋₆ haloalkyl, C₁-C₆ alkoxy, oxo, N(C₁-C₆alkyl)₂, NH₂, NH(C₁-C₆ alkyl), and hydroxy;
R³ is selected from cyano, hydroxy, C₁-C₆ alkoxy, C(O)OR′, C(O)OR¹⁴, C(O)R′, C(O)NR′R″, C(O)NHR¹⁴, S(O)₀₋₂R′, S(O)₀₋₂R¹⁴, S(O)₁₋₂NR′R″, S(O)₁₋₂NHR¹⁴, and R¹⁴—(C₀-C₂ alkylene)-, wherein the C₀-C₂ alkylene group is optionally substituted with one or more oxo;
R¹⁴ is C₃-C₅ cycloalkyl, 3- to 8-membered heterocycloalkyl, C₂-C₁₀ alkenyl, C₂-C₁₀ alkynyl, 5- to 10-membered monocyclic or bicyclic heteroaryl or C₆-C₁₀ monocyclic or bicyclic aryl, wherein each cycloalkyl, heterocycloalkyl, C₂-C₁₀ alkenyl, C₂-C₁₀ alkynyl, aryl or heteroaryl is optionally independently substituted with 1 or 2 R⁶;
each of R′ and R″ is independently hydrogen or C₁-C₆ alkyl;
R¹⁵ is —(Z⁴-Z⁵)ₐ₂-Z⁶;
a2 is an integer selected from 1-10 (e.g., 1-5);
each Z⁴ is independently selected from —O—, —S—, —NH—, and —N(C₁-C₃ alkyl)-;
provided that the Z⁴ group directly attached to R¹ or R² is —O— or —S—;
each Z⁵ is independently C₁-C₆ alkylene optionally substituted with one or more substituents independently selected from oxo, halo, and hydroxy; and
Z⁶ is OH, OC₁-C₆ alkyl, NH₂, NH(C₁-C₆ alkyl), N(C₁-C₆ alkyl)₂, NHC(O)(C₁-C₆ alkyl), NHC(O)(C₁-C₆ alkoxy), or an optionally substituted group selected from the group consisting of:
C₆-C₁₀ aryl, C₂-C₆ alkenyl, C₂-C₆ alkynyl, C₃-C₁₀ cycloalkyl, 5- to 10-membered heteroaryl, or 3- to 10-membered heterocycloalkyl, each of which is optionally substituted with one or more substituents independently selected from halo, C₁-C₆ alkyl, C₁₋₆ haloalkyl, C₁-C₆ alkoxy, oxo, N(C₁-C₆alkyl)₂, NH₂, NH(C₁-C₆ alkyl), and hydroxy;
or a pharmaceutically acceptable salt thereof;
provided that the compound is other than one or more compounds disclosed in WO 2018225018, which is incorporated herein by reference in its entirety; and
the compound is other than one or more compounds disclosed in IN 201721020305, which is incorporated herein by reference in its entirety.

2. A compound of Formula AA of claim 1 wherein the compound is an unequal mixture of: 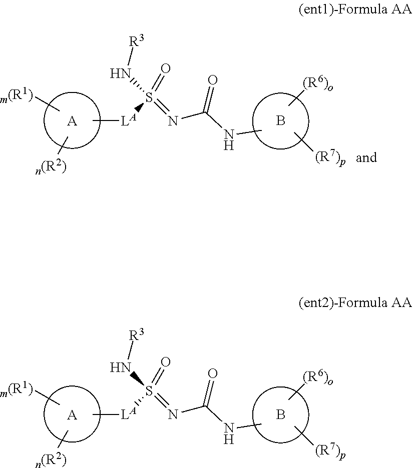
wherein
L^{A} is a bond or a C₁-C₈ alkylene optionally substituted with halo;
m=0, 1, or 2;
n=0, 1, or 2;
o=1 or 2;
p=0, 1, 2, or 3,
wherein
A is a 5- to 10-membered monocyclic or bicyclic heteroaryl or a C₆-C₁₀ monocyclic or bicyclic aryl;
B is a 5-10 membered monocyclic or bicyclic heteroaryl, or a C₆-C₁₀ monocyclic or bicyclic aryl; wherein at least one R⁶ is ortho to the bond connecting the B ring to the NH(CO) group of Formula AA;
R¹ and R² are each independently selected from C₁-C₆ alkyl, C₁-C₆ haloalkyl, C₁-C₆ alkoxy, C₁-C₆ haloalkoxy, halo, CN, NO₂, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NR⁸R⁹, C(O)R¹³, CONR⁸R⁹, SF₅, SC₁-C₆ alkyl, S(O₂)C₁-C₆ alkyl, S(O)C₁-C₆ alkyl, S(O₂)NR¹¹R¹², C₃-C₇ cycloalkyl, and 3- to 7-membered heterocycloalkyl,
wherein the C₁-C₆ alkyl, C₁-C₆ alkoxy, C₁-C₆ haloalkyl, C₆-C₁₀ aryl, 5- to 10-membered heteroaryl, C₃-C₇ cycloalkyl and 3- to 7-membered heterocycloalkyl is optionally substituted with one or more substituents each independently selected from hydroxy, halo, CN, oxo, C₁-C₆ alkyl, C₁-C₆ alkoxy, R¹⁵, C₁-C₆ haloalkoxy, NR⁸R⁹, ═NR¹⁰, COOC₁-C₆ alkyl, CONR⁸R⁹, 3- to 7-membered heterocycloalkyl, C₃-C₇ cycloalkyl, C₆-C₁₀ aryl, 5- to 10-membered heteroaryl, OCOC₁-C₆ alkyl, OCOC₆-C₁₀ aryl, OCO(5- to 10-membered heteroaryl), and OCO(3- to 7-membered heterocycloalkyl);
wherein each C₁-C₆ alkyl substituent and each C₁-C₆ alkoxy substituent of the R¹ or R² C₃-C₇ cycloalkyl or of the R¹ or R² 3- to 7-membered heterocycloalkyl is further optionally independently substituted with one to three hydroxy, —O(C₀-C₃ alkylene)C₆-C₁₀ aryl, halo, NR⁸R⁹, or oxo;
wherein the 3- to 7-membered heterocycloalkyl, C₆-C₁₀ aryl, and 5- to 10-membered heteroaryl are optionally substituted with one or more substituents independently selected from halo, C₁-C₆ alkyl, and OC₁-C₆ alkyl;
or one pair of R¹ and R² on adjacent atoms, taken together with the atoms connecting them, independently form one monocyclic or bicyclic C₄-C₁₂ carbocyclic ring or one monocyclic or bicyclic 5- to-12-membered heterocyclic ring containing 1-3 heteroatoms independently selected from O, N, and S, wherein the carbocyclic ring or heterocyclic ring is optionally independently substituted with one or more substituents each independently selected from hydroxy, halo, oxo, C₁-C₆ alkyl, C₂-C₆ alkenyl, C₂-C₆ alkynyl, C₁-C₆ alkoxy, OC₃-C₁₀ cycloalkyl, NR⁸R⁹, ═NR¹⁰, CN, COOC₁-C₆ alkyl, OS(O₂)C₆-C₁₀ aryl, S(O₂)C₆-C₁₀ aryl, C₆-C₁₀ aryl, 5- to 10-membered heteroaryl, C₃-C₁₀ cycloalkyl, 3- to 10-membered heterocycloalkyl, and CONR⁸R⁹,
wherein the C₁-C₆ alkyl, C₁-C₆ alkoxy, S(O₂)C₆-C₁₀ aryl, C₆-C₁₀ aryl, 5- to 10-membered heteroaryl, C₃-C₁₀ cycloalkyl, and 3- to 10-membered heterocycloalkyl are optionally substituted with one or more substituents each independently selected from hydroxy, halo, C₁-C₆ alkyl, C₂-C₆ alkenyl, C₂-C₆ alkynyl, C₃-C₁₀ cycloalkyl, C₁-C₆ alkoxy, oxo, NR⁸R⁹, ═NR¹⁰, COOC₁-C₆ alkyl, C₆-C₁₀ aryl, and CONR⁸R⁹;
R⁶ and R⁷ are each independently selected from C₁-C₆ alkyl, C₁-C₆ haloalkyl, C₁-C₆ alkoxy, C₁-C₆ haloalkoxy, halo, CN, NO₂, COC₁-C₆ alkyl, CO₂C₁-C₆ alkyl, CO₂C₃-C₈ cycloalkyl, OCOC₁-C₆ alkyl, OCOC₆-C₁₀ aryl, OCO(5- to 10-membered heteroaryl), OCO(3- to 7-membered heterocycloalkyl), C₆-C₁₀ aryl, 5- to 10-membered heteroaryl, NH₂, NHC₁-C₆ alkyl, N(C₁-C₆ alkyl)₂, CONR⁸R⁹, SF₅, SC₁-C₆ alkyl, S(O₂)C₁-C₆ alkyl, C₃-C₁₀ cycloalkyl, 3- to 10-membered heterocycloalkyl, and C₂-C₆ alkenyl,
wherein R⁶ and R⁷ are each optionally substituted with one or more substituents independently selected from hydroxy, halo, CN, oxo, C₁-C₆ alkyl, C₁-C₆ alkoxy, NR⁸R⁹, ═NR¹⁰, COOC₁-C₆ alkyl, CONR⁸R⁹, 3- to 7-membered heterocycloalkyl, C₆-C₁₀ aryl, 5- to 10-membered heteroaryl, OCOC₁-C₆ alkyl, OCOC₆-C₁₀ aryl, OCO(5- to 10-membered heteroaryl), OCO(3- to 7-membered heterocycloalkyl), C₆-C₁₀ aryloxy, and S(O₂)C₁-C₆ alkyl; and wherein the C₁-C₆ alkyl or C₁-C₆ alkoxy that R⁶ or R⁷ is substituted with is optionally substituted with one or more hydroxyl, C₆-C₁₀ aryl or NR⁸R⁹, or wherein R⁶ or R⁷ is optionally fused to a five- to -seven-membered carbocyclic ring or heterocyclic ring containing one or two heteroatoms independently selected from oxygen, sulfur and nitrogen;
wherein the 3- to 7-membered heterocycloalkyl, C₆-C₁₀ aryl, 5- to 10-membered heteroaryl are optionally substituted with one or more substituents independently selected from halo, C₁-C₆ alkyl, and OC₁-C₆ alkyl;
or at least one pair of R⁶ and R⁷ on adjacent atoms, taken together with the atoms connecting them, independently form at least one C₄-C₈ carbocyclic ring or at least one 5- to 8-membered heterocyclic ring containing 1 or 2 heteroatoms independently selected from O, N, and S, wherein the carbocyclic ring or heterocyclic ring is optionally independently substituted with one or more substituents independently selected from hydroxy, hydroxymethyl, halo, oxo, C₁-C₆ alkyl, C₁-C₆ alkoxy, NR⁸R⁹, CH₂NR⁸R⁹, ═NR¹⁰, COOC₁-C₆ alkyl, C₆-C₁₀ aryl, and CONR⁸R⁹;
R¹⁰ is C₁-C₆ alkyl;
each of R⁸ and R⁹ at each occurrence is independently selected from hydrogen, C₁-C₆ alkyl, C₂-C₆ alkenyl, C₃-C₇ cycloalkyl, (C═NR¹³)NR¹¹R¹², S(O₂)C₁-C₆ alkyl, S(O₂)NR¹¹R¹², COR¹³, CO₂R¹³ and CONR¹¹R¹²; wherein the C₁-C₆ alkyl is optionally substituted with one or more hydroxy, halo, C₁-C₆ alkoxy, C₆-C₁₀ aryl, 5- to 10-membered heteroaryl, C₃-C₇ cycloalkyl, 3- to 7-membered heterocycloalkyl, or NR¹¹R¹²;
or R⁸ and R⁹ taken together with the nitrogen they are attached to form a 3- to 10-membered monocyclic or bicyclic ring optionally containing one or more heteroatoms in addition to the nitrogen they are attached to, wherein the ring is optionally substituted with one or more substituents independently selected from halo, C₁-C₆ alkyl, C₁-C₆ haloalkyl, C₁-C₆ alkoxy, oxo, N(C₁-C₆alkyl)₂, NH₂, NH(C₁-C₆ alkyl), and hydroxy;
R¹³ is C₁-C₆ alkyl, C₁-C₆ haloalkyl, or —(Z¹-Z²)ₐ₁-Z³;
each of R¹¹ and R¹² at each occurrence is independently selected from hydrogen, C₁-C₆ alkyl, and —(Z¹-Z²)ₐ₁-Z³;
a1 is 0-10 (e.g., 0-4);
each Z¹ is independently C₁-C₆ alkylene optionally substituted with one or more substituents independently selected from oxo, halo, and hydroxy;
each Z² is independently a bond, NH, N(C₁-C₆ alkyl), —O—, —S—, or 5-10 membered heteroarylene;
Z³ is independently C₆-C₁₀ aryl, C₂-C₆ alkenyl, C₂-C₆ alkynyl, C₃-C₁₀ cycloalkyl, 5- to 10-membered heteroaryl, or 3- to 10-membered heterocycloalkyl, each of which is optionally substituted with one or more substituents independently selected from halo, C₁-C₆ alkyl, C₁₋₆ haloalkyl, C₁-C₆ alkoxy, oxo, N(C₁-C₆alkyl)₂, NH₂, NH(C₁-C₆ alkyl), and hydroxy;
R³ is selected from cyano, hydroxy, C₁-C₆ alkoxy, C(O)OR′, C(O)OR¹⁴, C(O)R′, C(O)NR′R″, C(O)NHR¹⁴, S(O)₀₋₂R′, S(O)₀₋₂R¹⁴, S(O)₁₋₂NR′R″, S(O)₁₋₂NHR¹⁴, and R¹⁴—(C₀-C₂ alkylene)-, wherein the C₀-C₂ alkylene group is optionally substituted with one or more oxo;
R¹⁴ is C₃-C₅ cycloalkyl, 3- to 8-membered heterocycloalkyl, C₂-C₁₀ alkenyl, C₂-C₁₀ alkynyl, 5- to 10-membered monocyclic or bicyclic heteroaryl or C₆-C₁₀ monocyclic or bicyclic aryl, wherein each cycloalkyl, heterocycloalkyl, C₂-C₁₀ alkenyl, C₂-C₁₀ alkynyl, aryl or heteroaryl is optionally independently substituted with 1 or 2 R⁶;
each of R′ and R″ is independently hydrogen or C₁-C₆ alkyl;
R¹⁵ is —(Z⁴-Z⁵)ₐ₂-Z⁶;
a2 is an integer selected from 1-10 (e.g., 1-5);
each Z⁴ is independently selected from —O—, —S—, —NH—, and —N(C₁-C₃ alkyl)-;
provided that the Z⁴ group directly attached to R¹ or R² is —O— or —S—;
each Z⁵ is independently C₁-C₆ alkylene optionally substituted with one or more substituents independently selected from oxo, halo, and hydroxy; and
Z⁶ is OH, OC₁-C₆ alkyl, NH₂, NH(C₁-C₆ alkyl), N(C₁-C₆ alkyl)₂, NHC(O)(C₁-C₆ alkyl), NHC(O)(C₁-C₆ alkoxy), or an optionally substituted group selected from the group consisting of:
C₆-C₁₀ aryl, C₂-C₆ alkenyl, C₂-C₆ alkynyl, C₃-C₁₀ cycloalkyl, 5- to 10-membered heteroaryl, or 3- to 10-membered heterocycloalkyl, each of which is optionally substituted with one or more substituents independently selected from halo, C₁-C₆ alkyl, C₁₋₆ haloalkyl, C₁-C₆ alkoxy, oxo, N(C₁-C₆alkyl)₂, NH₂, NH(C₁-C₆ alkyl), and hydroxy;
or a pharmaceutically acceptable salt thereof.

3. The compound of claim 1, wherein the compound is a compound of (ent1)-Formula AA.

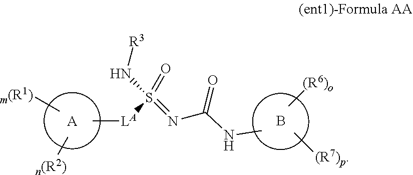

4. The compound of claim 1, wherein the compound is a compound of (ent2)-Formula AA.

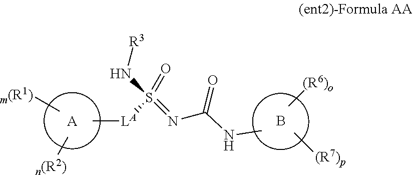

5. The compound of any preceding claim, wherein L^{A} is a bond.

6. The compound of any preceding claim, wherein R³ is methyl, or CN.

7. The compound of any preceding claim, wherein A is a 5- to 6-membered monocyclic heteroaryl optionally substituted with 1 or 2 R¹ and optionally substituted with 1 or 2 R².

8. The compound of any preceding claim, wherein one or more R¹ is independently selected from 1-amino-2-hydroxy-prop-2-yl; 1-acetamido-2-hydroxy-prop-2-yl; 1-(tert-butoxycarbonyl)amino-2-hydroxy-prop-2-yl; 1-(2-hydroxyethoxy)-2-hydroxy-2-propyl; 1-(2-benzyloxyethoxy)-2-hydroxy-2-propyl; and 1-(2-methoxyethoxy)-2-hydroxy-2-propyl.

9. The compound of any preceding claim, wherein B is phenyl or pyridine, substituted with 1 or 2 R⁶ and optionally substituted with 1, 2, or 3 R⁷.

10. The compound of any preceding claim, wherein at least one pair of R⁶ and R⁷ taken together with the atoms connecting them independently form a C₅ carbocyclic ring, wherein the carbocyclic ring is optionally substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹, and wherein the other pair of R⁶ and R⁷ taken together with the atoms connecting them independently form a C₅ carbocyclic ring, wherein the carbocyclic ring is optionally substituted with one or more substituents independently selected from hydroxy, halo, oxo, C₁-C₆ alkyl, C₁-C₆ alkoxy, NR⁸R⁹, ═NR¹⁰, COOC₁-C₆ alkyl, C₆-C₁₀ aryl, and CONR⁸R⁹.

11. The compound of claims 1 to 8, wherein B is selected from:

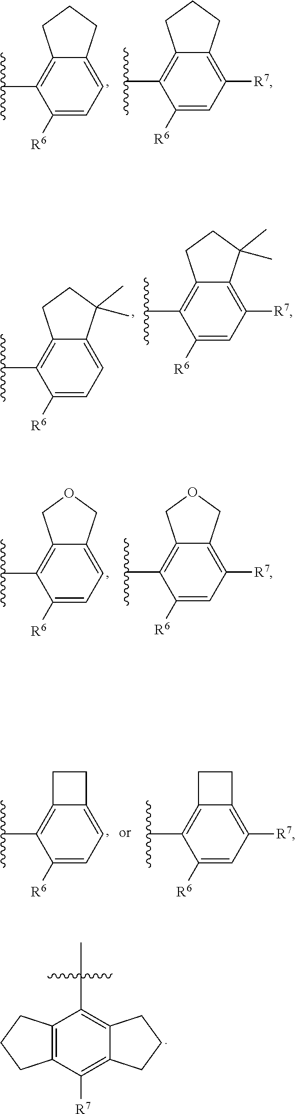

12. The compound of claims 1 to 8, wherein B is: 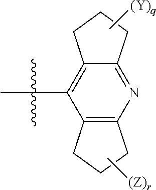
wherein
q is 0, 1, or 2; r is 0, 1, or 2; wherein each of Y and Z is independently selected from C₁-C₆ alkyl and hydroxy; or wherein when two Y are attached to the same carbon, the two Y are taken together with the carbon they are attached to to form a cyclopropyl ring; or wherein when two Z are attached to the same carbon, the two Z are taken together with the carbon they are attached to form a cyclopropyl ring; or B is: 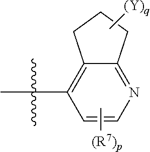
wherein
R⁷ is selected from C₁-C₆ alkyl, C₆-C₁₀ aryl, and C₃-C₁₀ cycloalkyl; p is 0, 1, or 2; q is 0, 1, or 2; wherein each Y is independently selected from C₁-C₆ and hydroxy; or when two Y are attached to the same carbon, the two Y are taken together with the carbon they are attached to to form a cyclopropyl ring.

13. A pharmaceutical composition comprising a compound or salt as claimed in any one of claims 1- and one or more pharmaceutically acceptable excipients.

14. A compound of claims 1 to 12 for use in the treatment of disease modulated by NLRP3.

15. A compound claims 1 to 12 for use in the treatment of disease modulated by TNFα.
